(19) United States
(12) Patent Application Publication
(10) Pub. No.: US 2021/0122730 A1
(43) Pub. Date: Apr. 29, 2021

(54) GLUCOCORTICOID RECEPTOR MODULATORS

(71) Applicant: ORIC Pharmaceuticals, Inc., South San Francisco, CA (US)

(72) Inventors: Xiaohui DU, South San Francisco, CA (US); John EKSTEROWICZ, South San Francisco, CA (US); Valeria R. FANTIN, South San Francisco, CA (US); Yosup REW, South San Francisco, CA (US); Daqing SUN, South San Francisco, CA (US); Qiuping YE, South San Francisco, CA (US); Haiying ZHOU, South San Francisco, CA (US); Hiroyuki KAWAI, South San Francisco, CA (US); Jared MOORE, South San Francisco, CA (US); Johnny PHAM, South San Francisco, CA (US); Kejia WU, South San Francisco, CA (US); Liusheng ZHU, South San Francisco, CA (US); Dennis YAMASHITA, South San Francisco, CA (US)

(21) Appl. No.: 16/604,543

(22) PCT Filed: Apr. 10, 2018
(86) PCT No.: PCT/US2018/026928

§ 371 Date: Oct. 10, 2019

Related U.S. Application Data
(60) Provisional application No. 62/484,335, filed on Apr. 11, 2017.
(60) Provisional application No. 62/555,604, filed on Sep. 7, 2017.

(51) Int. Cl. C07D 401/12; C07D 417/12; C07D 401/14; C07D 417/14; C07D 405/14; A61K 31/4439; A61K 31/4545; A61K 31/444; A61K 31/427; A61K 31/337; A61K 31/404; A61K 31/58; A61K 31/473; A61K 31/277; A61K 31/567; A61K 31/4164; A61K 31/167; A61K 31/496; A61K 31/18; A61K 31/4166; A61K 45/06; A61K 35/17; A61K 39/00; A61K 39/395

(52) U.S. Cl. CPC C07D 401/12; A61K 2039/505; C07D 401/14; C07D 417/14; C07D 405/14; A61K 31/4439; A61K 31/4545; A61K 31/444; A61K 31/427; A61K 31/337; A61K 31/404; A61K 31/58; A61K 31/473; A61K 31/277; A61K 31/567; A61K 31/4164; A61K 31/167; A61K 31/496; A61K 31/18; A61K 31/4166; A61K 45/06; A61K 35/17; A61K 39/0011; A61K 39/3955; C07D 417/12

(57) ABSTRACT

Described herein are glucocorticoid receptor modulators and pharmaceutical compositions comprising said compounds. The subject compounds and compositions are useful for the treatment of cancer and hypercortisolism.

### CROSS-REFERENCE TO RELATED APPLICATIONS

This application claims the benefit of U.S. Provisional Application Ser. No. 62/484,335, filed Apr. 11, 2017 and U.S. Provisional Application Ser. No. 62/555,604, filed Sep. 7, 2017, each of which are hereby incorporated by reference in their entirety.

### BACKGROUND

A need exists in the art for an effective treatment of cancer, neoplastic disease, and hypercortisolism.

### BRIEF SUMMARY OF THE INVENTION

Provided herein are compounds of Formula (I), (Ia)-(Ic), (II), (IIa)-(IIc), (III), or (IIIa)-(IIIc), and pharmaceutical compositions comprising said compounds. The subject compounds and compositions are useful as glucocorticoid receptor (GR) modulators. Furthermore, the subject compounds and compositions are useful for the treatment of cancer, such as prostate cancer, breast cancer, lung cancer, ovarian cancer, and hypercortisolism.

Some embodiments provided herein describe compounds having the structure of Formula (I), or a pharmaceutically acceptable salt, solvate, or stereoisomer thereof:

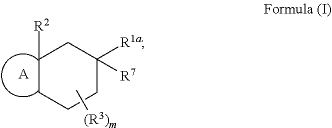

wherein:

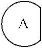

is

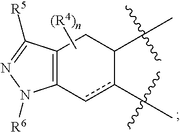

[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹,—S(O)₂CH₂R¹⁷, or —S(O)₂R¹;
R² is hydrogen, halogen, alkyl, alkenyl, —CN, —OR⁸, —NR⁸R⁹, cycloalkyl, heterocycloalkyl, aryl, heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein alkyl, alkenyl, cycloalkyl, heterocycloalkyl, aryl, and heteroaryl are optionally substituted with one, two, or three R^{12b};
each R³ and each R⁴ is independently halogen or alkyl;
R⁵ is hydrogen, alkyl, or haloalkyl;
R⁶ is aryl, heteroaryl, cycloalkyl, or heterocycloalkyl, wherein aryl, heteroaryl, cycloalkyl, and heterocycloalkyl are optionally substituted with one, two, or three R^{12c};
R⁷ is hydrogen, halogen, —CN, alkyl, haloalkyl, heteroalkyl, alkenyl, —OR⁸, —NR⁸R⁹, cycloalkyl, or heterocycloalkyl;
each R⁸ and each R⁹ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12d};
   or R⁸ and R⁹ are taken together with the atom to which they are attached to form a heterocycloalkyl optionally substituted with one, two, or three R^{12d};
R¹⁰ is hydrogen or alkyl;
R¹¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12e}
each R^{12a}, R^{12b}, R^{12c}, R^{12d}, R^{12e}, R^{12f}, and R^{12g} is independently selected from halogen, —CN, alkyl, haloalkyl, —OR¹³, -alkyl-OR¹³, —NR¹³R¹⁴, -alkyl-NR¹³R¹⁴, cycloalkyl, -alkyl-cycloalkyl, heterocycloalkyl, aryl, heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein cycloalkyl, heterocycloalkyl, aryl, and heteroaryl are optionally substituted with one, two, or three groups selected from halogen, alkyl, and haloalkyl;
each R¹³ and each R¹⁴ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
   or R¹³ and R¹⁴ are taken together with the atom to which they are attached to form a heterocycloalkyl optionally substituted with one, two, or three groups selected from halogen, alkyl, and haloalkyl;
each R¹⁵ is independently alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl;
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
m is 0, 1, 2, 3, or 4; and
n is 0, 1, 2, or 3.

In some embodiments of a compound of Formula (I), or a pharmaceutically acceptable salt, solvate, or stereoisomer thereof:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
each R³ and each R⁴ is independently halogen or C₁₋₆alkyl;
R⁵ is hydrogen, C₁₋₆alkyl, or C₁₋₆haloalkyl;
R⁶ is C₆₋₁₀aryl, C₂₋₉heteroaryl, C₃₋₈cycloalkyl, or C₂₋₉heterocycloalkyl, wherein C₆₋₁₀aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R¹²;
or R⁸ and R⁹ are taken together with the atom to which they are attached to form a C₂₋₉heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
   or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
R¹⁸ and R¹⁹ is each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12g};
R²⁰ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
m is 0, 1, 2, 3, or 4; and
n is 0, 1, 2, or 3.

In some embodiments of a compound of Formula (I), or a pharmaceutically acceptable salt, solvate, or stereoisomer thereof, R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, or —S(O)₂CH₂R¹⁷.

In some embodiments of a compound of Formula (I), or a pharmaceutically acceptable salt, solvate, or stereoisomer thereof, R^{1a} is —NR¹⁶C(O)R¹⁷, —NR¹⁶S(O)₂R¹⁷, —S(O)₂NR¹⁸R¹⁹, or —C(R²⁰)₂S(O)₂R¹⁷.

In some embodiments of a compound of Formula (I), or a pharmaceutically acceptable salt, solvate, or stereoisomer thereof, the compound of Formula (I) has the structure of Formula (II):

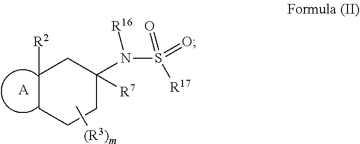

wherein:

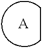

is

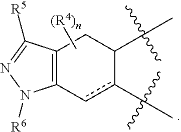

In some embodiments of a compound of Formula (I), or a pharmaceutically acceptable salt, solvate, or stereoisomer thereof, R^{1a} is —NR¹⁶C(O)R¹⁷.

In some embodiments of a compound of Formula (I), or a pharmaceutically acceptable salt, solvate, or stereoisomer thereof, R¹⁶ is C₁₋₆alkyl or C₃₋₈cycloalkyl, wherein C₁₋₆alkyl and C₃₋₈cycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵.

In some embodiments of a compound of Formula (I), or a pharmaceutically acceptable salt, solvate, or stereoisomer thereof, R¹⁶ is unsubstituted C₁₋₆alkyl.

In some embodiments of a compound of Formula (I), or a pharmaceutically acceptable salt, solvate, or stereoisomer thereof, R¹⁶ is unsubstituted C₃₋₈cycloalkyl.

In some embodiments of a compound of Formula (I), or a pharmaceutically acceptable salt, solvate, or stereoisomer thereof, R¹⁶ is unsubstituted cyclopropyl.

In some embodiments of a compound of Formula (I), or a pharmaceutically acceptable salt, solvate, or stereoisomer thereof, R^{1a} is —C(R²⁰)₂S(O)₂R¹⁷.

In some embodiments of a compound of Formula (I), or a pharmaceutically acceptable salt, solvate, or stereoisomer thereof, R²⁰ is hydrogen, C₁₋₆alkyl, or C₃₋₈cycloalkyl.

In some embodiments of a compound of Formula (I), or a pharmaceutically acceptable salt, solvate, or stereoisomer thereof, R¹⁷ is C₆₋₁₀aryl or C₂₋₉heteroaryl, and the C₆₋₁₀aryl and C₂₋₉heteroaryl are optionally substituted with one, two, or three R^{12f}.

In some embodiments of a compound of Formula (I), or a pharmaceutically acceptable salt, solvate, or stereoisomer thereof, R¹⁷ is phenyl optionally substituted with one, two, or three R^{12f}.

In some embodiments of a compound of Formula (I), or a pharmaceutically acceptable salt, solvate, or stereoisomer thereof, R¹⁷ is phenyl optionally substituted with one, two, or three groups selected from halogen, C₁₋₆alkyl, and C₁₋₆haloalkyl.

In some embodiments of a compound of Formula (I), or a pharmaceutically acceptable salt, solvate, or stereoisomer thereof, R¹⁷ is C₂₋₉heteroaryl optionally substituted with one, two, or three R^{12f}.

In some embodiments of a compound of Formula (I), or a pharmaceutically acceptable salt, solvate, or stereoisomer thereof, R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}.

In some embodiments of a compound of Formula (I), or a pharmaceutically acceptable salt, solvate, or stereoisomer thereof, R¹⁷ is selected from pyrazole, triazole, and pyridine, wherein pyrazole, triazole, and pyridine are optionally substituted with one, two, or three groups selected from halogen, C₁₋₆alkyl, and C₁₋₆haloalkyl.

In some embodiments of a compound of Formula (I), or a pharmaceutically acceptable salt, solvate, or stereoisomer thereof, R^{1a} is —S(O)₂NR¹⁸R¹⁹.

In some embodiments of a compound of Formula (I), or a pharmaceutically acceptable salt, solvate, or stereoisomer thereof, R¹⁸ and R¹⁹ is each independently hydrogen, C₁₋₆alkyl, C₆₋₁₀aryl, or C₂₋₉heteroaryl, wherein C₁₋₆alkyl, C₆₋₁₀ aryl, and C₂₋₉heteroaryl are optionally substituted with one, two, or three R^{12g}.

In some embodiments of a compound of Formula (I), or a pharmaceutically acceptable salt, solvate, or stereoisomer thereof, R¹⁸ and R¹⁹ is each independently hydrogen, C₁₋₆alkyl, C₆₋₁₀aryl, or C₂₋₉heteroaryl, wherein C₁₋₆alkyl, C₆₋₁₀ aryl, and C₂₋₉heteroaryl are optionally substituted with one or two groups selected from halogen, C₁₋₆alkyl, and C₁₋₆haloalkyl.

In some embodiments of a compound of Formula (I), or a pharmaceutically acceptable salt, solvate, or stereoisomer thereof, R¹⁸ is C₁₋₆alkyl, and R¹⁹ is C₆₋₁₀aryl or C₂₋₉heteroaryl, wherein C₆₋₁₀ aryl and C₂₋₉heteroaryl are optionally substituted with one or two groups selected from halogen, C₁₋₆alkyl, and C₁₋₆haloalkyl.

In some embodiments of a compound of Formula (I), or a pharmaceutically acceptable salt, solvate, or stereoisomer thereof, the compound of Formula (I) has the structure of Formula (III):

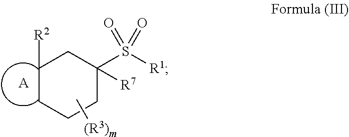

wherein:

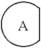

is

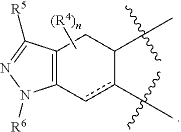

In some embodiments of a compound of Formula (I), or a pharmaceutically acceptable salt, solvate, or stereoisomer thereof, R¹ is C₆₋₁₀aryl or C₂₋₉heteroaryl, and the C₆₋₁₀aryl and C₂₋₉heteroaryl are optionally substituted with one, two, or three R^{12a}.

In some embodiments of a compound of Formula (I), or a pharmaceutically acceptable salt, solvate, or stereoisomer thereof, R¹ is phenyl optionally substituted with one, two, or three R^{12a}.

In some embodiments of a compound of Formula (I), or a pharmaceutically acceptable salt, solvate, or stereoisomer thereof, R¹ is phenyl optionally substituted with one, two, or three groups selected from halogen, C₁₋₆alkyl, and C₁₋₆haloalkyl.

In some embodiments of a compound of Formula (I), or a pharmaceutically acceptable salt, solvate, or stereoisomer thereof, R¹ is C₂₋₉heteroaryl optionally substituted with one, two, or three R^{12a}.

In some embodiments of a compound of Formula (I), or a pharmaceutically acceptable salt, solvate, or stereoisomer thereof, R¹ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12a}.

In some embodiments of a compound of Formula (I), or a pharmaceutically acceptable salt, solvate, or stereoisomer thereof, R¹ is selected from pyrazole, triazole, and pyridine, wherein pyrazole, triazole, and pyridine are optionally substituted with one, two, or three groups selected from halogen, C₁₋₆alkyl, and C₁₋₆haloalkyl.

In some embodiments of a compound of Formula (I) or (III), or a pharmaceutically acceptable salt, solvate, or stereoisomer thereof, R⁶ is phenyl optionally substituted with one, two, or three R^{12c}.

In some embodiments of a compound of Formula (I) or (III), or a pharmaceutically acceptable salt, solvate, or stereoisomer thereof, R⁶ is phenyl optionally substituted with one, two, or three groups selected from halogen, C₁₋₆alkyl, and C₁₋₆haloalkyl.

In some embodiments of a compound of Formula (I) or (III), or a pharmaceutically acceptable salt, solvate, or stereoisomer thereof, R⁶ is phenyl substituted with one or two groups selected from halogen, C₁₋₆alkyl, and C₁₋₆haloalkyl.

In some embodiments of a compound of Formula (I) or (III), or a pharmaceutically acceptable salt, solvate, or stereoisomer thereof, R⁶ is phenyl substituted with a halogen.

In some embodiments of a compound of Formula (I) or (III), or a pharmaceutically acceptable salt, solvate, or stereoisomer thereof, R² is C₁₋₆alkyl, C₂₋₆alkenyl, C₃₋₈cycloalkyl, or —C(O)R¹¹, wherein C₁₋₆alkyl, C₂₋₆alkenyl, and C₃₋₈cycloalkyl are optionally substituted with one, two, or three R^{12b}.

In some embodiments of a compound of Formula (I) or (III), or a pharmaceutically acceptable salt, solvate, or stereoisomer thereof, R² is C₁₋₆alkyl, C₂₋₆alkenyl, or C₃₋₈cycloalkyl, wherein C₁₋₆alkyl, C₂₋₆alkenyl, and C₃₋₈cycloalkyl are substituted with one, two, or three groups selected from halogen, C₁₋₆alkyl, C₁₋₆haloalkyl, —OR¹³, —NR¹³R¹⁴, and C₂₋₉heteroaryl.

In some embodiments of a compound of Formula (I) or (III), or a pharmaceutically acceptable salt, solvate, or stereoisomer thereof, R² is C₁₋₆alkyl substituted with one, two, or three groups selected from halogen, —OR¹³, —NR¹³R¹⁴, and C₂₋₉heteroaryl.

In some embodiments of a compound of Formula (I) or (III), or a pharmaceutically acceptable salt, solvate, or stereoisomer thereof, R² is C₂₋₆alkenyl or C₃₋₈cycloalkyl, wherein C₂₋₆alkenyl and C₃₋₈cycloalkyl are substituted with a C₂₋₉heteroaryl.

In some embodiments of a compound of Formula (I) or (III), or a pharmaceutically acceptable salt, solvate, or stereoisomer thereof, R² is —C(O)R¹¹.

In some embodiments of a compound of Formula (I) or (III), or a pharmaceutically acceptable salt, solvate, or stereoisomer thereof, R¹¹ is C₆₋₁₀aryl or C₂₋₉heteroaryl, wherein C₆₋₁₀ aryl and C₂₋₉heteroaryl are optionally substituted with one, two, or three R^{12e}.

In some embodiments of a compound of Formula (I) or (III), or a pharmaceutically acceptable salt, solvate, or stereoisomer thereof, R¹¹ is C₂₋₉heteroaryl optionally substituted with one or two R^{12e}.

In some embodiments of a compound of Formula (I) or (III), or a pharmaceutically acceptable salt, solvate, or stereoisomer thereof, R¹¹ is C₂₋₉heteroaryl optionally substituted with one or two groups selected from halogen, C₁₋₆alkyl, C₁₋₆haloalkyl, and C₃₋₈cycloalkyl.

In some embodiments of a compound of Formula (I) or (III), or a pharmaceutically acceptable salt, solvate, or stereoisomer thereof, R¹¹ is selected from thiazole and pyridine, wherein thiazole and pyridine are optionally substituted with one or two groups selected from halogen, C₁₋₆alkyl, C₁₋₆haloalkyl, and C₃₋₈cycloalkyl.

In some embodiments of a compound of Formula (I) or (III), or a pharmaceutically acceptable salt, solvate, or stereoisomer thereof, R¹¹ is selected from unsubstituted thiazole and unsubstituted pyridine.

In some embodiments of a compound of Formula (I) or (III), or a pharmaceutically acceptable salt, solvate, or stereoisomer thereof, R⁷ is hydrogen, halogen, or C₁₋₆ alkyl.

In some embodiments of a compound of Formula (I) or (III), or a pharmaceutically acceptable salt, solvate, or stereoisomer thereof, R⁷ is hydrogen.

In some embodiments of a compound of Formula (I) or (III), or a pharmaceutically acceptable salt, solvate, or stereoisomer thereof, m is 0. In some embodiments of a compound of Formula (I) or (III), or a pharmaceutically acceptable salt, solvate, or stereoisomer thereof, n is 0.

In some embodiments of a compound of Formula (I) or (III), or a pharmaceutically acceptable salt, solvate, or stereoisomer thereof, R⁵ is hydrogen.

Also disclosed herein is a pharmaceutical composition comprising a compound disclosed herein, or a pharmaceutically acceptable salt, solvate, or stereoisomer thereof, and at least one pharmaceutically acceptable excipient.

Also disclosed herein is method for treating or preventing cancer in a subject, the method comprising administering a therapeutically effective amount of a compound disclosed herein, or a pharmaceutically acceptable salt, solvate, or stereoisomer thereof, to the subject in need thereof. Also disclosed herein is a method of reducing incidences of cancer recurrence, the method comprising administering to a subject in cancer remission a therapeutically effective amount of a compound disclosed herein, or a pharmaceutically acceptable salt, solvate, or stereoisomer thereof. Also disclosed herein is a method for treating a therapy-resistant cancer in a subject, the method comprising administering a therapeutically effective amount of a compound disclosed herein, or a pharmaceutically acceptable salt, solvate, or stereoisomer thereof, to the subject in need thereof. In some embodiments of a method disclosed herein, the cancer is triple negative breast cancer, ovarian cancer, castration resistant prostate cancer, or doubly resistant prostate cancer. In some embodiments of a method disclosed herein, the cancer is non-small cell lung cancer, clear renal cell carcinoma, hepatocellular carcinoma, melanoma, or bladder cancer. In some embodiments of a method disclosed herein, the method further comprises administering one or more additional therapeutic agents to the subject. In some embodiments of a method disclosed herein, the one or more additional therapeutic agents are androgen receptor signaling inhibitors. In some embodiments of a method disclosed herein, the androgen receptor signaling inhibitor is 3,3′-diindolylmethane (DIM), abiraterone acetate, apalutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof. In some embodiments of a method disclosed herein, the one or more additional therapeutic agents are chemotherapeutic agents. In some embodiments of a method disclosed herein, the chemotherapeutic agents are cisplatin, carboplatin, paclitaxel, docetaxel, nab-paclitaxel, gemcitabine, doxorubicin, camptothecin, topotecan, pemetrexed, or a combination thereof. In some embodiments of a method disclosed herein, the one or more additional therapeutic agents are anti-PD-L1 agents or anti-PD1 agents, anti-CTLA-4 agents, CAR-T cells therapy, cancer vaccines, or IDO-1 inhibitors.

Also disclosed herein is a method for treating a hypercortisolism disease or disorder in a subject, the method comprising administering a therapeutically effective amount a compound disclosed herein, or a pharmaceutically acceptable salt, solvate, or stereoisomer thereof, to the subject in need thereof. In some embodiments of a method disclosed herein, the hypercortisolism disease or disorder is Cushing's syndrome.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)₁R^{f} (where t is 1 or 2), —S(O)ₜOR^{a} (where t is 1 or 2), —S(O)₁R^{f} (where t is 1 or 2), and —S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2), and —S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R a, —N(R^{a})S(O)ₜR^{a} (where t is 1 or 2), —S(O)OR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula -Rd-aryl where Rd is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —Re-aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O-Rc-aryl where Re is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R-carbocyclyl where R is an alkylene chain as defined above. The alkylene chain and the carbocyclyl radical are optionally substituted as defined above.

“Carbocyclylalkynyl” refers to a radical of the formula —R-carbocyclyl where R is an alkynylene chain as defined above. The alkynylene chain and the carbocyclyl radical are optionally substituted as defined above.

“Carbocyclylalkoxy” refers to a radical bonded through an oxygen atom of the formula —O—R-carbocyclyl where R is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

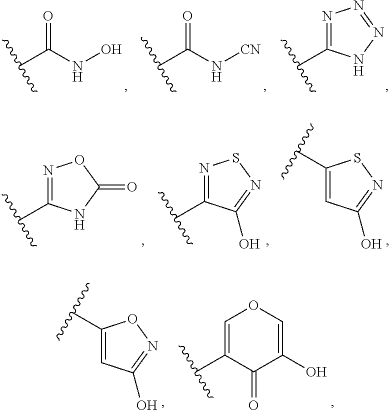

and the like.

“Deuteroalkyl” refers to an alkyl group where 1 or more hydrogen atoms of an alkyl are replaced with deuterium.

“Halo” or “halogen” refers to bromo, chloro, fluoro or iodo substituents.

“Haloalkyl” refers to an alkyl radical, as defined above, that is substituted by one or more halo radicals, as defined above, for example, trifluoromethyl, difluoromethyl, fluoromethyl, 2,2,2-trifluoroethyl, 1-fluoromethyl-2-fluoroethyl, trichloromethyl, dichloromethyl, chloromethyl, 2,2,2-trichloroethyl, 1-chloromethyl-2-chloroethyl, tribromomethyl, dibromomethyl, bromomethyl, 2,2,2-tribromoethyl, 1-bromomethyl-2-bromoethyl, and the like. In some embodiments, the alkyl part of the haloalkyl radical is optionally substituted as defined above for an alkyl group.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆heteroalkyl wherein the heteroalkyl comprises 1 to 6 carbons and one or more oxygen, nitrogen (e.g. —NH—, —N(alkyl)-), or sulfur. Unless stated otherwise specifically in the specification, an heteroalkyl chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Heterocyclyl” refers to a stable 3-to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula -Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O-Rc-heterocyclyl where Rc is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula -Rc-heteroaryl, where Re is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O-Rc-heteroaryl, where Re is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments described herein,

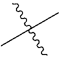

designates the attachment point of a chemical moiety. For example, in the compounds of Formula (III)

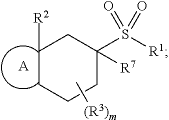

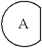

is

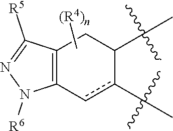

and is attached to form

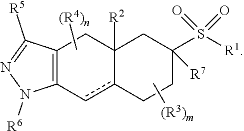

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

The compounds described herein may exhibit their natural isotopic abundance, or one or more of the atoms may be artificially enriched in a particular isotope having the same atomic number, but an atomic mass or mass number different from the atomic mass or mass number predominantly found in nature.

All isotopic variations of the compounds of the present invention, whether radioactive or not, are encompassed within the scope of the present invention. For example, hydrogen has three naturally occurring isotopes, denoted 1H (protium), 2H (deuterium), and 3H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art or by processes analogous to those described in the Schemes and Examples herein using appropriate isotopically-enriched reagents and/or intermediates. In some embodiments, the compounds described herein contain one or more isotopic variants (e.g., deuterium, tritium, 13C, and/or 14C).

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

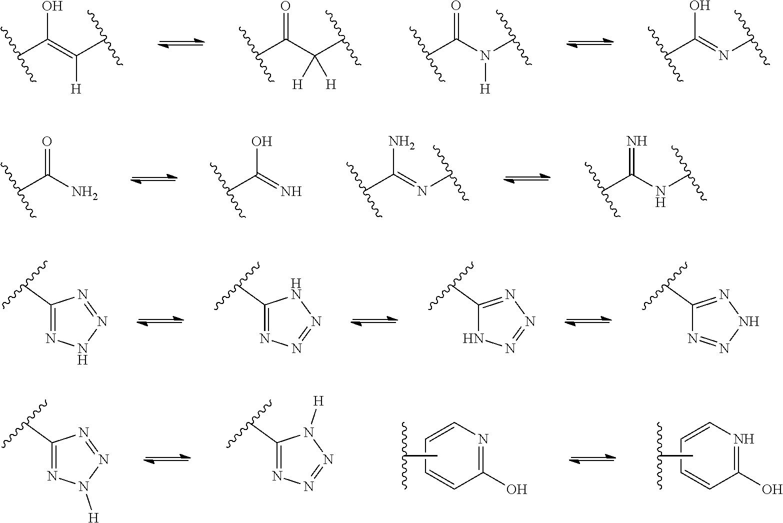

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

Abbreviations used herein have their conventional meaning within the chemical and biological arts. The following abbreviations have the indicated meaning throughout: Na₂HPO₄=disodium phosphate, AcOH=acetic acid, aq.=aqueous, NH4Cl=ammonium chloride, DCM=dichloromethane, DMPU=1,3-dimethyl-3,4,5,6-tetrahydro-2(1H)-pyrimidinone, ESI=electrospray ionization, EtOAc=ethyl acetate, g=gram, h=hour, LCMS=liquid chromatography mass spectrometry, LDA=lithium diisopropylamide, MgSO₄=magnesium sulfate, m/s=mass-to-charge ratio, mg=milligram, MeOH=methanol, min=minute, NMR=nuclear magnetic resonance, RT or rt=room temperature, sat.=saturated, NaHCO₃=sodium bicarbonate, NaBH₄=sodium borohydride, Na₂CO₃=sodium carbonate, NaCl=sodium chloride, Na₂SO₄=sodium sulfate, Na₂S₂O₃=sodium thiosulfate, TFA=trifluoroacetic acid, and THF=tetrahydrofuran.

### Compounds

Described herein are compounds of Formulas (I), (Ia), (Ib), (Ic), (II), (IIa), (IIb), (IIc), (III), (IIIa), (IIIb), and (IIIc) that are GR modulators. These compounds, and compositions comprising these compounds, are useful for the treatment of cancer, neoplastic disease, and hypercortisolism diseases and disorders.

In some embodiments provided herein is a compound having the structure of Formula (I), or a pharmaceutically acceptable salt, solvate, or stereoisomer thereof:

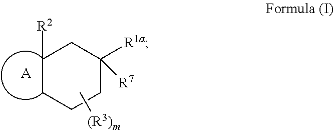

wherein:

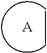

is

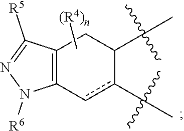

[symbol] is a single bond or a double bond;
R^{1a} is —NR¹⁶C(O)R¹⁷, —NR¹⁶S(O)₂R¹⁷, —S(O)₂NR¹⁸R¹⁹, —CH₂S(O)₂R¹⁷, —C(O)NR¹⁸R¹⁹, —C(R²⁰)₂S(O)₂R¹⁷, or —S(O)₂R¹;
R² is hydrogen, halogen, alkyl, alkenyl, —CN, —OR⁸, —NR⁸R⁹, cycloalkyl, heterocycloalkyl, aryl, heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein alkyl, alkenyl, cycloalkyl, heterocycloalkyl, aryl, and heteroaryl are optionally substituted with one, two, or three R^{12b};
each R³ and each R⁴ is independently halogen or alkyl;
R⁵ is hydrogen, alkyl, or haloalkyl;
R⁶ is aryl, heteroaryl, cycloalkyl, or heterocycloalkyl, wherein aryl, heteroaryl, cycloalkyl, and heterocycloalkyl are optionally substituted with one, two, or three R^{12c};
R⁷ is hydrogen, halogen, —CN, alkyl, haloalkyl, heteroalkyl, alkenyl, —OR⁸, —NR⁸R⁹, cycloalkyl, or heterocycloalkyl;
each R⁸ and each R⁹ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or alkyl;
R¹¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, alkyl, haloalkyl, —OR¹³, -alkyl-OR¹³, —NR¹³R¹⁴, -alkyl-NR¹³R¹⁴, cycloalkyl, -alkyl-cycloalkyl, heterocycloalkyl, aryl, heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein cycloalkyl, heterocycloalkyl, aryl, and heteroaryl are optionally substituted with one, two, or three groups selected from halogen, alkyl, and haloalkyl;
each R¹³ and each R¹⁴ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
or R¹³ and R¹⁴ are taken together with the atom to which they are attached to form a heterocycloalkyl optionally substituted with one, two, or three groups selected from halogen, alkyl, and haloalkyl;
each R¹⁵ is independently alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl;
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
m is 0, 1, 2, 3, or 4; and
n is 0, 1, 2, or 3.

In other embodiments provided herein is a compound having the structure of Formula (I), or a pharmaceutically acceptable salt, solvate, or stereoisomer thereof:

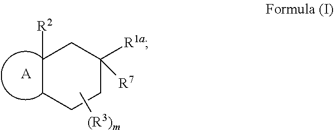

wherein:

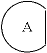

is

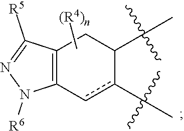

[symbol] is a single bond or a double bond;
R^{1a} is —NR¹⁶C(O)R¹⁷, —NR¹⁶S(O)₂R¹⁷, —S(O)₂NR¹⁸R¹⁹, —CH₂S(O)₂R¹⁷, —C(O)NR¹⁸R¹⁹, —C(R²⁰)₂S(O)₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
each R³ and each R⁴ is independently halogen or C₁₋₆alkyl;
R⁵ is hydrogen, C₁₋₆alkyl, or C₁₋₆haloalkyl;
R⁶ is C₆₋₁₀aryl, C₂₋₉heteroaryl, C₃₋₈cycloalkyl, or C₂₋₉heterocycloalkyl, wherein C₆₋₁₀aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
R¹⁸ and R¹⁹ is each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12g};
R²⁰ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
m is 0, 1, 2, 3, or 4; and
n is 0, 1, 2, or 3.

In some embodiments,
[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is C₆₋₁₀aryl optionally substituted with one, two, or three R^{12c};
R⁷ is hydrogen, halogen, or C₁₋₆alkyl;
R¹¹ is C₂₋₉heteroaryl optionally substituted with one, two, or three R^{12e};
each R^{12a}, R^{12c}, R^{12e}, R^{12f}, and R^{12g} is independently selected from halogen, C₁₋₆alkyl, C₁₋₆haloalkyl, —OR¹³, C₃₋₈cycloalkyl, or —C₁₋₆alkyl-C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
each R¹⁵ is independently C₁₋₆alkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f};
R¹⁸ is C₁₋₆alkyl or C₃₋₈cycloalkyl;
R¹⁹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12g};
R²⁰ is hydrogen, C₁₋₆alkyl, or C₃₋₈cycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
m is 0; and
n is 0.

In some embodiments,
[symbol] is a single bond or a double bond;
R^{1a} is —NR¹⁶S(O)₂R¹⁷, —C(R²⁰)₂S(O)₂R¹⁷, or —S(O)₂R¹;
R² is —C(O)R¹¹;
each R³ and each R⁴ is independently halogen or C₁₋₆alkyl;
R⁵ is hydrogen;
R⁶ is C₆₋₁₀aryl optionally substituted with one, two, or three R¹²;
R⁷ is hydrogen, halogen, or C₁₋₆alkyl;
R¹¹ is C₂₋₉heteroaryl optionally substituted with one, two, or three R^{12e};
each R^{12a}, R^{12c}, R^{12e}, and R^{12f} is independently selected from halogen, C₁₋₆alkyl, C₁₋₆haloalkyl, —OR¹³, C₃₋₈cycloalkyl, or —C₁₋₆alkyl-C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
each R¹⁵ is independently C₁₋₆alkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
R²⁰ is hydrogen, C₁₋₆alkyl, or C₃₋₈cycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
m is 0; and

n is 0. In some embodiments,
[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is C₆₋₁₀aryl optionally substituted with one, two, or three R^{12c};
R⁷ is hydrogen, halogen, or C₁₋₆alkyl;
R¹¹ is C₂₋₉heteroaryl optionally substituted with one, two, or three R^{12e};
each R^{12a}, R^{12c}, R^{12e}, R^{12f}, and R^{12g} is independently selected from halogen, C₁₋₆alkyl, C₁₋₆haloalkyl, —OR¹³, C₃₋₈cycloalkyl, or —C₁₋₆alkyl-C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
each R¹⁵ is independently C₁₋₆alkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
R¹⁸ is C₁₋₆alkyl or C₃₋₈cycloalkyl;
R¹⁹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12g};
R²⁰ is hydrogen, C₁₋₆alkyl, or C₃₋₈cycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
m is 0; and
n is 0.

In some embodiments,
[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is phenyl substituted with halogen;
R⁷ is hydrogen, halogen, or C₁₋₆alkyl;
R¹¹ is C₂₋₉heteroaryl optionally substituted with one, two, or three R^{12e};
each R^{12e} is independently selected from halogen, C₁₋₆alkyl, C₁₋₆haloalkyl, —OR¹³, or C₃₋₈cycloalkyl;
each R¹³ is independently hydrogen or C₁₋₆alkyl, wherein C₁₋₆alkyl is optionally substituted with one, two, or three groups selected from halogen and C₁₋₆haloalkyl;
each R¹⁵ is independently C₁₋₆alkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹¹;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
each R^{12f} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three groups selected from halogen and C₁₋₆haloalkyl;
R¹⁸ is C₁₋₆alkyl or C₃₋₈cycloalkyl;
R¹⁹ is C₆₋₁₀ aryl, or C₂₋₉heteroaryl, wherein C₆₋₁₀ aryl, and C₂₋₉heteroaryl are optionally substituted with alkyl;
R²⁰ is hydrogen, C₁₋₆alkyl, or C₃₋₈cycloalkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
each R^{12a} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
m is 0; and
n is 0.

In some embodiments,
[symbol] is a double bond;
R^{1a} is —NR¹⁶C(O)R¹⁷, —NR¹⁶S(O)₂R¹⁷, —S(O)₂NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is phenyl substituted with halogen;
R⁷ is hydrogen;
R¹¹ is C₂₋₉heteroaryl optionally substituted with one, two, or three R^{12e};
each R^{12e} is independently halogen, C₁₋₆alkyl, C₁₋₆haloalkyl, —OR¹³, or C₃₋₈cycloalkyl;
each R¹³ is independently hydrogen or C₁₋₆alkyl, wherein C₁₋₆alkyl is optionally substituted with one, two, or three groups halogens;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, and —C(O)OR¹³;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f};
each R^{12f} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three halogen;
R¹⁸ is C₁₋₆alkyl or C₃₋₈cycloalkyl;
R¹⁹ is C₆₋₁₀ aryl or C₂₋₉heteroaryl, wherein C₆₋₁₀ aryl, and C₂₋₉heteroaryl are optionally substituted with alkyl;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
each R^{12a} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three halogen;
m is 0; and
n is 0.

In some embodiments,
[symbol] is a double bond;
R^{1a} is —NR¹⁶C(O)R¹⁷, —NR¹⁶S(O)₂R¹⁷, —S(O)₂NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen;
R¹¹ is thiazole or pyridine, wherein the thiazole or pyridine is optionally substituted with one, two, or three R^{12e};
each R^{12e} is independently selected from halogen, C₁₋₆alkyl, C₁₋₆haloalkyl, —OR¹³, or C₃₋₆cycloalkyl;
each R¹³ is independently hydrogen or C₁₋₆alkyl, wherein C₁₋₆alkyl is optionally substituted with one, two, or three fluoros;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, and —C(O)OR¹³;
R¹⁷ is C₃₋₆alkyl, C₃₋₆cycloalkyl, C₄₋₆heterocycloalkyl, phenyl, or C₂₋₉heteroaryl, wherein C₃₋₆alkyl, C₃₋₆cycloalkyl, C₄₋₆heterocycloalkyl, phenyl, and C₂₋₉heteroaryl are optionally substituted with one, two, or three R^{12f};
each R^{12f} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three halogen;
R¹⁸ is C₁₋₃alkyl or C₃₋₆cycloalkyl;
R¹⁹ is phenyl or C₂₋₉heteroaryl, wherein phenyl and C₂₋₉heteroaryl are optionally substituted with alkyl;
R¹ is C₁₋₆alkyl, C₃₋₆cycloalkyl, C₂₋₆heterocycloalkyl, phenyl, or C₂₋₉heteroaryl, wherein C₁₋₆alkyl, C₃₋₆cycloalkyl, C₂₋₆heterocycloalkyl, phenyl, and C₂₋₉heteroaryl are optionally substituted with one, two, or three R^{12a};
each R^{12a} is independently halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three halogen;
m is 0; and
n is 0.

In some embodiments,
[symbol] is a double bond;
R^{1a} is —NR¹⁶C(O)R¹⁷, —NR¹⁶S(O)₂R¹⁷, —S(O)₂NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen;
R¹¹ is thiazole or pyridine, wherein the thiazole or pyridine is optionally substituted with one, two, or three R^{12e};
each R^{12e} is independently —CF₃, methyl, cyclopropyl, Cl, F, methoxy, or —OCF₃;
R¹⁶ is

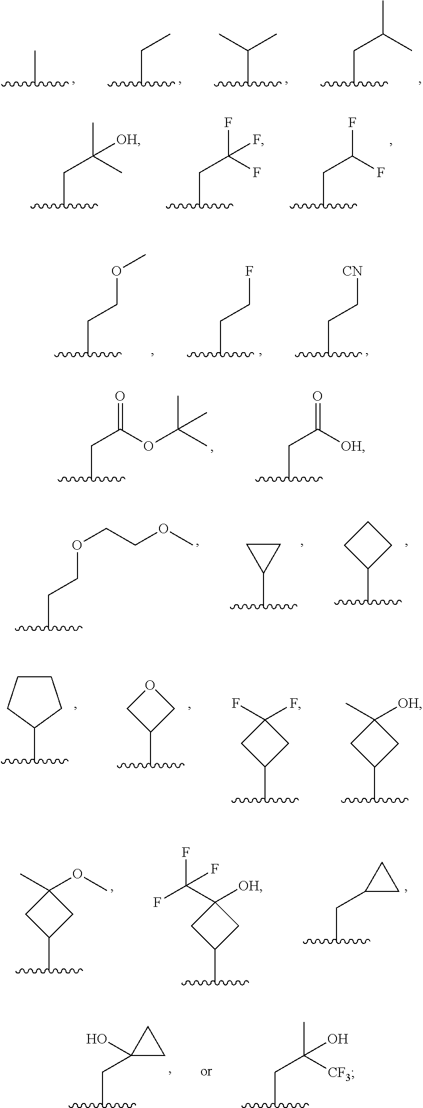

R¹⁷ is propyl, iso-propyl, butyl, iso-butyl, t-butyl, pentyl, tetrahydropyran, cyclopropyl, cyclopentyl, cyclohexyl, phenyl, pyrazole, pyridine, thiadiazole, or triazole, wherein R¹ is optionally substituted with one, two, or three R^{12f};
each R^{12f} is independently methyl ethyl, propyl, iso-propyl, butyl, t-butyl, fluoro, chloro, methoxy, methylcyclopropyl, C₁₋₃ fluoroalkyl, or OH;
R¹⁸ is methyl, ethyl, cyclopropyl, or methylcyclopropyl;
R¹⁹ is phenyl, pyrazole, pyridine, thiadiazole, or triazole, wherein R¹⁹ is optionally substituted with alkyl;
R¹ is propyl, iso-propyl, butyl, iso-butyl, t-butyl, pentyl, tetrahydropyran, cyclopropyl, cyclopentyl, cyclohexyl, phenyl, pyrazole, pyridine, thiadiazole, or triazole, wherein R¹ is optionally substituted with one, two, or three R^{12a};
each R^{12a} is independently methyl, ethyl, propyl, iso-propyl, butyl, t-butyl, fluoro, chloro, methoxy, methylcyclopropyl, C₁₋₃ fluoroalkyl, or OH;
m is 0; and
n is 0.

In some embodiments,
[symbol] is a double bond;
R^{1a} is —NR¹⁶C(O)R¹⁷, —NR¹⁶S(O)₂R¹⁷, —S(O)₂NR¹⁸R¹⁹, —S(O)₂CH₂R¹⁷, or —S(O)₂R¹;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen;
R¹¹ is thiazole or pyridine, wherein the thiazole or pyridine is optionally substituted with one, two, or three R^{12e};
each R^{12e} is independently —CF₃, methyl, cyclopropyl, Cl, F, methoxy, or —OCF₃;
R¹⁶ is methyl, ethyl, cyclopropyl, or methylcyclopropyl;
R¹⁷ is propyl, iso-propyl, butyl, iso-butyl, t-butyl, pentyl, tetrahydropyran, cyclopropyl, cyclopentyl, cyclohexyl, phenyl, pyrazole, pyridine, thiadiazole, or triazole, wherein R¹⁷ is optionally substituted with one, two, or three R^{12f};
each R^{12f} is independently methyl ethyl, propyl, iso-propyl, butyl, t-butyl, fluoro, chloro, methoxy, methylcyclopropyl, C₁₋₃ fluoroalkyl, or OH;
R¹⁸ is methyl, ethyl, cyclopropyl, or methylcyclopropyl;
R¹⁹ is phenyl, pyrazole, pyridine, thiadiazole, or triazole, wherein R¹⁹ is optionally substituted with alkyl;
R¹ is propyl, iso-propyl, butyl, iso-butyl, t-butyl, pentyl, tetrahydropyran, cyclopropyl, cyclopentyl, cyclohexyl, phenyl, pyrazole, pyridine, thiadiazole, or triazole, wherein R¹ is optionally substituted with one, two, or three R^{12a};
each R^{12a} is independently methyl, ethyl, propyl, iso-propyl, butyl, t-butyl, fluoro, chloro, methoxy, methylcyclopropyl, C₁₋₃ fluoroalkyl, or OH;
m is 0; and
n is 0.

In some embodiments, a compound of Formula (I), or a pharmaceutically acceptable salt, solvate, or stereoisomer thereof, has the structure:

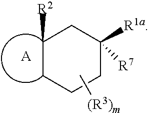

In some embodiments, a compound of Formula (I), or a pharmaceutically acceptable salt, solvate, or stereoisomer thereof, has the structure:

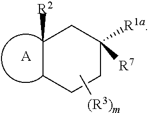

In some embodiments, a compound of Formula (I), or a pharmaceutically acceptable salt, solvate, or stereoisomer thereof, has the structure:

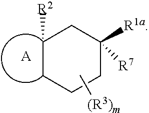

In some embodiments, a compound of Formula (I), or a pharmaceutically acceptable salt, solvate, or stereoisomer thereof, has the structure:

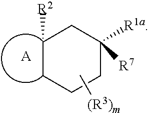

In some embodiments provided herein is a compound having the structure of Formula (Ia), or a pharmaceutically acceptable salt, solvate, or stereoisomer thereof:

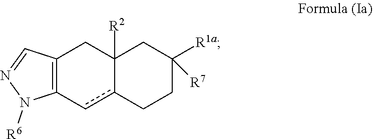

wherein:
[symbol] is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀ aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀ aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀ aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12d};
or R⁸ and R⁹ are taken together with the atom to which they are attached to form a C₂₋₉heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹¹, —C(O)OR¹, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹¹ is independently C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};

R¹⁸ and R¹⁹ is each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12g};
R²⁰ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl; and
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a}.

In some embodiments, a compound of Formula (Ia), or a pharmaceutically acceptable salt, solvate, or stereoisomer thereof, has the structure:

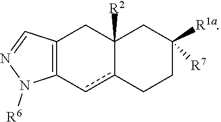

In some embodiments, a compound of Formula (Ia), or a pharmaceutically acceptable salt, solvate, or stereoisomer thereof, has the structure:

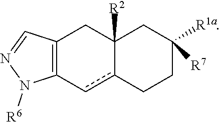

In some embodiments, a compound of Formula (Ia), or a pharmaceutically acceptable salt, solvate, or stereoisomer thereof, has the structure:

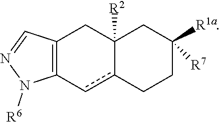

In some embodiments, a compound of Formula (Ia), or a pharmaceutically acceptable salt, solvate, or stereoisomer thereof, has the structure:

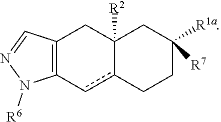

In some embodiments provided herein is a compound having the structure of Formula (Ib), or a pharmaceutically acceptable salt, solvate, or stereoisomer thereof:

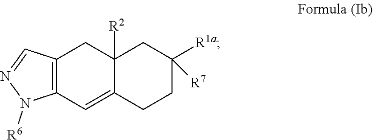

wherein:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀ aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀ aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀ aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f};
R¹⁸ and R¹⁹ is each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl; and
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a}.

In some embodiments, a compound of Formula (Ib), or a pharmaceutically acceptable salt, solvate, or stereoisomer thereof, has the structure:

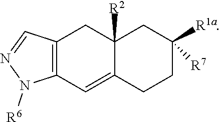

In some embodiments, a compound of Formula (Ib), or a pharmaceutically acceptable salt, solvate, or stereoisomer thereof, has the structure:

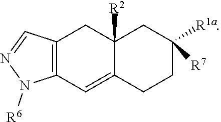

In some embodiments, a compound of Formula (Ib), or a pharmaceutically acceptable salt, solvate, or stereoisomer thereof, has the structure:

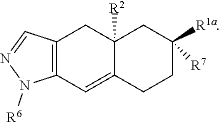

In some embodiments, a compound of Formula (Ib), or a pharmaceutically acceptable salt, solvate, or stereoisomer thereof, has the structure:

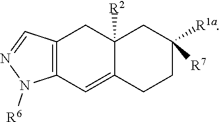

In some embodiments provided herein is a compound having the structure of Formula (Ic), or a pharmaceutically acceptable salt, solvate, or stereoisomer thereof:

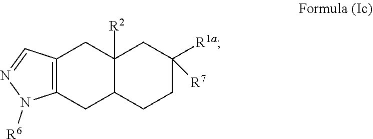

wherein:
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀ aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12d};
or R⁸ and R⁹ are taken together with the atom to which they are attached to form a C₂₋₉heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, R^{12e}, R^{12f}, and R^{12g} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀aryl, C₂₋₉heteroaryl, —C(O)R¹¹, —C(O)OR¹, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
   or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f};
R¹⁸ and R¹⁹ is each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12g};
R²⁰ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl; and
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a}.

In some embodiments, a compound of Formula (Ic), or a pharmaceutically acceptable salt, solvate, or stereoisomer thereof, has the structure:

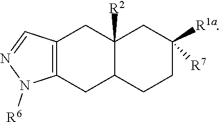

In some embodiments, a compound of Formula (Ic), or a pharmaceutically acceptable salt, solvate, or stereoisomer thereof, has the structure:

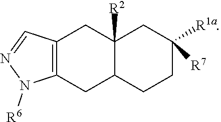

In some embodiments, a compound of Formula (Ic), or a pharmaceutically acceptable salt, solvate, or stereoisomer thereof, has the structure:

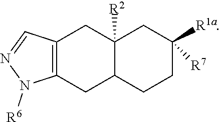

In some embodiments, a compound of Formula (Ic), or a pharmaceutically acceptable salt, solvate, or stereoisomer thereof, has the structure:

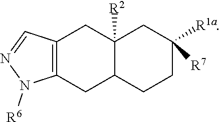

In some embodiments or a compound of Formula (I), (Ia), (Ib), or (Ic), R^{1a} is-S(O)₂R¹ and R¹ is C₁₋₆alkyl optionally substituted with one, two, or three R^{2a}. In some embodiments or a compound of Formula (I), (Ia), (Ib), or (Ic), R^{1a} is-S(O)₂R¹⁷ and R¹⁷ is C₁₋₆alkyl substituted with phenyl, wherein phenyl is optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —S(O)₂R¹⁷ and R¹⁷ is C₁₋₆alkyl substituted with phenyl, wherein phenyl is unsubstituted. In some embodiments or a compound of Formula (I), (Ia), (Ib), or (Ic), R^{1a} is-S(O)₂R¹⁷ and R¹⁷ is C₁₋₆alkyl substituted with phenyl, wherein phenyl is substituted with one, two, or three groups selected from halogen, C₁₋₆alkyl, and C₁₋₆haloalkyl.

In some embodiments or a compound of Formula (I), (Ia), (Ib), or (Ic), R^{1a} is —NR¹⁶C(O)R¹⁷, —NR¹⁶S(O)₂R¹⁷, —S(O)₂NR¹⁸R¹⁹, —CH₂S(O)₂R¹⁷, —C(O)NR¹⁸R¹⁹, or —S(O)₂CH₂R¹⁷. In some embodiments or a compound of Formula (I), (Ia), (b), or (Ic), R^{1a} is —NR¹⁶C(O)R¹⁷, —NR¹⁶S(O)₂R¹⁷, —S(O)₂NR¹⁸R¹⁹, or —CH₂S(O)₂R¹⁷. In some embodiments or a compound of Formula (I), (Ia), (Ib), or (Ic), R^{1a} is —NR¹⁶C(O)R¹⁷ or —NR¹⁶S(O)₂R¹⁷. In some embodiments or a compound of Formula (I), (Ia), (Ib), or (Ic), R^{1a} is —NR¹⁶S(O)₂R¹⁷ or —S(O)₂NR¹⁸R¹⁹. In some embodiments or a compound of Formula (I), (Ia), (Ib), or (Ic), R^{1a} is —C(O)NR¹⁸R¹⁹ or —S(O)₂CH₂R¹⁷. In some embodiments or a compound of Formula (I), (Ia), (Ib), or (Ic), R^{1a} is —NR¹⁶C(O)R¹⁷ or —C(O)NR¹⁸R¹⁹. In some embodiments or a compound of Formula (I), (Ia), (Ib), or (Ic), R^{1a} is —CH₂S(O)₂R¹⁷ or —S(O)₂CH₂R¹⁷.

In some embodiments or a compound of Formula (I), (Ia), (Ib), or (Ic), R^{1a} is —NR¹⁶S(O)₂R¹⁷. In some embodiments or a compound of Formula (I), (Ia), (b), or (Ic), R^{1a} is —NR¹⁶S(O)₂R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴. In some embodiments or a compound of Formula (I), (Ia), (Ib), or (Ic), R^{1a} is —NR¹⁶S(O)₂R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R. In some embodiments or a compound of Formula (I), (Ia), (Ib), or (Ic), R^{1a} is —NR¹⁶S(O)₂R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵, R¹³ is hydrogen or C₁₋₆alkyl, and R¹⁵ is C₁₋₆alkyl. In some embodiments or a compound of Formula (I), (Ia), (Ib), or (Ic), R^{1a} is —NR¹⁶S(O)₂R¹⁷ and R¹⁶ is

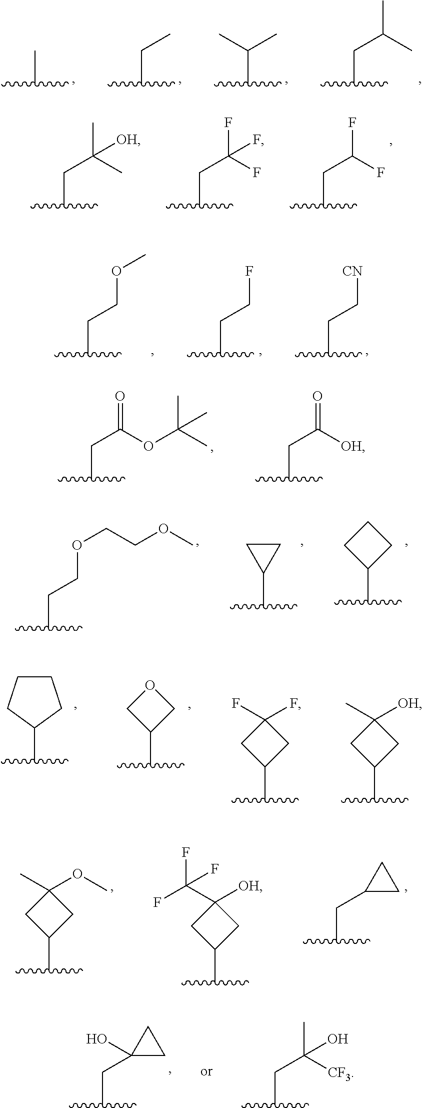

In some embodiments or a compound of Formula (I), (Ia), (Ib), or (Ic), R^{1a} is —NR¹⁶S(O)₂R¹⁷ and R¹⁶ is unsubstituted C₁₋₆alkyl. In some embodiments or a compound of Formula (I), (Ia), (Ib), or (Ic), R^{1a} is —NR¹⁶S(O)₂R¹⁷ and R¹⁶ is —CH₃. In some embodiments or a compound of Formula (I), (Ia), (Ib), or (Ic), R^{1a} is —NR¹⁶S(O)₂R¹⁷ and R¹⁶ is unsubstituted C₃₋₈cycloalkyl. In some embodiments or a compound of Formula (I), (Ia), (Ib), or (Ic), R^{1a} is —NR¹⁶S(O)₂R¹⁷ and R¹⁶ is unsubstituted cyclopropyl. In some embodiments or a compound of Formula (I), (Ia), (Ib), or (Ic), R^{1a} is —NR¹⁶S(O)₂R¹⁷ and R¹⁶ is unsubstituted methylcyclopropyl. In some embodiments or a compound of Formula (I), (Ia), (Ib), or (Ic), R^{1a} is —NR¹⁶S(O)₂R¹⁷ and R¹⁷ is C₆₋₁₀ aryl or C₂₋₉heteroaryl, wherein C₆₋₁₀aryl and C₂₋₉heteroaryl are optionally substituted with one, two, or three R^{12f}. In some embodiments or a compound of Formula (I), (Ia), (Ib), or (Ic), R^{1a} is —NR¹⁶S(O)₂R¹⁷ and R¹⁷ is phenyl optionally substituted with one, two, or three R^{12f}. In some embodiments or a compound of Formula (I), (Ia), (Ib), or (Ic), R^{1a} is —NR¹⁶S(O)₂R¹⁷ and R¹⁷ is phenyl optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —NR¹⁶S(O)₂R¹⁷ and R¹⁷ is C₂₋₉heteroaryl optionally substituted with one, two, or three R¹². In some embodiments or a compound of Formula (I), (Ia), (Ib), or (Ic), R^{1a} is —NR¹⁶S(O)₂R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}. In some embodiments or a compound of Formula (I), (Ia), (Ib), or (Ic), R^{1a} is —NR¹⁶S(O)₂R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three groups selected from halogen, C₁₋₆alkyl, and C₁₋₆haloalkyl.

In some embodiments or a compound of Formula (I), (Ia), (Ib), or (Ic), R^{1a} is —NR¹⁶C(O)R¹⁷. In some embodiments or a compound of Formula (I), (Ia), (Ib), or (Ic), R^{1a} is —NR¹⁶C(O)R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹¹, —NR¹³R¹⁴, —C(O)R¹⁵, —C(O)OR¹³, —C(O)NR¹³R¹⁴, —NR¹³C(O)R⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴. In some embodiments or a compound of Formula (I), (Ia), (Ib), or (Ic), R^{1a} is —NR¹⁶C(O)R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R. In some embodiments or a compound of Formula (I), (Ia), (Ib), or (Ic), R^{1a} is —NR¹⁶C(O)R¹⁷ and R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR³, —C(O)OR¹³, and —S(O)₂R¹¹, R¹³ is hydrogen or C₁₋₆alkyl, and R¹¹ is C₁₋₆alkyl. In some embodiments or a compound of Formula (I), (Ia), (Ib), or (Ic), R^{1a} is —NR¹⁶C(O)R¹⁷ and R¹⁶ is

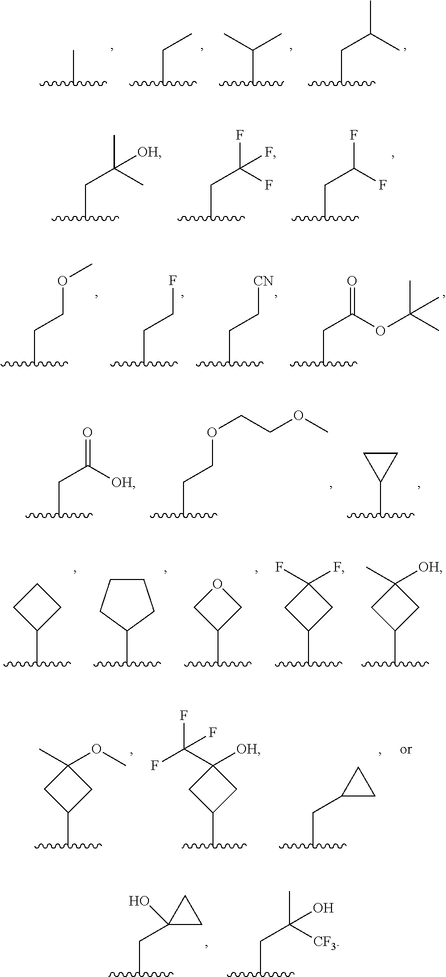

In some embodiments or a compound of Formula (I), (Ia), (Ib), or (Ic), R^{1a} is —NR¹⁶C(O)R¹⁷ and R¹⁶ is unsubstituted C₁₋₆alkyl. In some embodiments or a compound of Formula (I), (Ia), (Ib), or (Ic), R^{1a} is —NR¹⁶C(O)R¹⁷ and R¹⁶ is —CH₃. In some embodiments or a compound of Formula (I), (Ia), (Ib), or (Ic), R^{1a} is —NR¹⁶C(O)R¹⁷ and R¹⁶ is unsubstituted C₃₋₈cycloalkyl. In some embodiments or a compound of Formula (I), (Ia), (Ib), or (Ic), R^{1a} is —NR¹⁶C(O)R¹⁷ and R¹⁶ is unsubstituted cyclopropyl. In some embodiments or a compound of Formula (I), (Ia), (Ib), or (Ic), R^{1a} is —NR¹⁶C(O)R¹⁷ and R¹⁷ is C₆₋₁₀aryl or C₂₋₉heteroaryl, wherein C₆₋₁₀aryl and C₂₋₉heteroaryl are optionally substituted with one, two, or three R^{12f}. In some embodiments or a compound of Formula (I), (Ia), (Ib), or (Ic), R^{1a} is —NR¹⁶C(O)R¹⁷ and R¹⁷ is phenyl optionally substituted with one, two, or three R². In some embodiments or a compound of Formula (I), (Ia), (Ib), or (Ic), R^{1a} is —NR¹⁶C(O)R¹⁷ and R¹⁷ is phenyl optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —NR¹⁶C(O)R¹⁷ and R¹⁷ is C₂₋₉heteroaryl optionally substituted with one, two, or three R^{12f}. In some embodiments or a compound of Formula (I), (Ia), (Ib), or (Ic), R^{1a} is —NR¹⁶C(O)R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}. In some embodiments or a compound of Formula (I), (Ia), (Ib), or (Ic), R^{1a} is —NR¹⁶C(O)R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three groups selected from halogen, C₁₋₆alkyl, and C₁₋₆haloalkyl.

In some embodiments or a compound of Formula (I), (Ia), (Ib), or (Ic), R^{1a} is —C(R²⁰)₂S(O)₂R¹⁷, each R² is independently hydrogen, C₁₋₆alkyl, or C₃₋₈cycloalkyl, and R⁷ is C₆₋₁₀aryl or C₂₋₉heteroaryl, wherein C₆₋₁₀aryl and C₂₋₉heteroaryl are optionally substituted with one, two, or three R^{12f}some embodiments or a compound of Formula (I), (Ia), (Ib), or (Ic), R^{1a} is —CH₂S(O)₂R¹⁷. In some embodiments or a compound of Formula (I), (Ia), (Ib), or (Ic), R^{1a} is —CH₂S(O)₂R¹⁷ and R¹⁷ is C₆₋₁₀aryl or C₂₋₉heteroaryl, wherein C₆₋₁₀aryl and C₂₋₉heteroaryl are optionally substituted with one, two, or three R^{12f}. In some embodiments or a compound of Formula (I), (Ia), (Ib), or (Ic), R^{1a} is —CH₂S(O)₂R¹⁷ and R¹⁷ is phenyl optionally substituted with one, two, or three R^{12f}. In some embodiments or a compound of Formula (I), (Ia), (Ib), or (Ic), R^{1a} is —CH₂S(O)₂R¹⁷ and R¹⁷ is phenyl optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —CH₂S(O)₂R¹⁷ and R¹⁷ is C₂₋₉heteroaryl optionally substituted with one, two, or three R^{12f}. In some embodiments or a compound of Formula (I), (Ia), (Ib), or (Ic), R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}. In some embodiments or a compound of Formula (I), (Ia), (Ib), or (Ic), R^{1a} is —CH₂S(O)₂R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹. In some embodiments or a compound of Formula (I), (Ia), (Ib), or (Ic), R^{1a} is —S(O)₂NR¹⁸R¹⁹ and R¹⁸ and R¹⁹ is each independently hydrogen, C₁₋₆alkyl, C₆₋₁₀ aryl, or C₂₋₉heteroaryl, wherein C₁₋₆alkyl, C₆₋₁₀ aryl, and C₂₋₉heteroaryl are optionally substituted with one, two, or three R^{12g}. In some embodiments or a compound of Formula (I), (Ia), (Ib), or (Ic), R^{1a} is —S(O)₂NR¹⁸R¹⁹ and R¹⁸ and R¹⁹ is each independently hydrogen, C₁₋₆alkyl, C₆₋₁₀aryl, or C₂₋₉heteroaryl, wherein C₁₋₆alkyl, C₆₋₁₀ aryl, and C₂₋₉heteroaryl are optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹, R¹⁸ is C₁₋₆alkyl, and R¹⁹ is C₆₋₁₀ aryl or C₂₋₉heteroaryl, wherein C₆₋₁₀aryl and C₂₋₉heteroaryl are optionally substituted with one or two groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹, R¹⁸ is C₁₋₆alkyl, and R¹⁹ is C₆₋₁₀aryl optionally substituted with one or two groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹, R¹⁸ is —CH₃, and R¹⁹ is C₆₋₁₀aryl optionally substituted with one or two groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹, R¹⁸ is C₁₋₆alkyl, and R¹⁹ is C₂₋₉heteroaryl optionally substituted with one or two groups selected from halogen, C₁₋₆alkyl, and C₁₋₆haloalkyl. In some embodiments or a compound of Formula (I), (Ia), (Ib), or (Ic), R^{1a} is —S(O)₂NR¹⁸R¹⁹, R¹⁸ is —CH₃, and R¹⁹ is C₂₋₉heteroaryl optionally substituted with one or two groups selected from halogen, C₁₋₆alkyl, and C₁₋₆haloalkyl.

In some embodiments or a compound of Formula (I), (Ia), (Ib), or (Ic), R^{1a} is —S(O)₂CH₂R¹⁷. In some embodiments or a compound of Formula (I), (Ia), (Ib), or (Ic), R^{1a} is —S(O)₂CH₂R¹⁷ and R¹⁷ is C₆₋₁₀aryl or C₂₋₉heteroaryl, wherein C₆₋₁₀aryl and C₂₋₉heteroaryl are optionally substituted with one, two, or three R^{12f}. In some embodiments or a compound of Formula (I), (Ia), (Ib), or (Ic), R^{1a} is —S(O)₂CH₂R¹⁷ and R¹⁷ is phenyl optionally substituted with one, two, or three R^{12f} In some embodiments or a compound of Formula (I), (Ia), (Ib), or (Ic), R^{1a} is —S(O)₂CH₂R¹⁷ and R¹⁷ is phenyl optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —S(O)₂CH₂R¹⁷ and R¹⁷ is C₂₋₉heteroaryl optionally substituted with one, two, or three R^{12f}. In some embodiments or a compound of Formula (I), (Ia), (Ib), or (Ic), R⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f} In some embodiments or a compound of Formula (I), (Ia), (Ib), or (Ic), R^{1a} is —S(O)₂CH₂R¹⁷ and R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹. In some embodiments or a compound of Formula (I), (Ia), (Ib), or (Ic), R^{1a} is —C(O)NR¹⁸R¹⁹ and R¹⁸ and R¹⁹ is each independently hydrogen, C₁₋₆alkyl, C₆₋₁₀aryl, or C₂₋₉heteroaryl, wherein C₁₋₆alkyl, C₆₋₁₀ aryl, and C₂₋₉heteroaryl are optionally substituted with one, two, or three R^{12g}. In some embodiments or a compound of Formula (I), (Ia), (Ib), or (Ic), R^{1a} is —C(O)NR¹⁸R¹⁹ and R¹⁸ and R¹⁹ is each independently hydrogen, C₁₋₆alkyl, C₆₋₁₀aryl, or C₂₋₉heteroaryl, wherein C₁₋₆alkyl, C₆₋₁₀ aryl, and C₂₋₉heteroaryl are optionally substituted with one, two, or three groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹, R¹⁸ is C₁₋₆alkyl, and R¹⁹ is C₆₋₁₀aryl or C₂₋₉heteroaryl, wherein C₆₋₁₀aryl and C₂₋₉heteroaryl are optionally substituted with one or two groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹, R¹⁸ is C₁₋₆alkyl, and R¹⁹ is C₆₋₁₀aryl optionally substituted with one or two groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹, R¹⁸ is —CH₃, and R¹⁹ is C₆₋₁₀aryl optionally substituted with one or two groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹, R¹⁸ is C₁₋₆alkyl, and R¹ is C₂₋₉heteroaryl optionally substituted with one or two groups selected from halogen, C₁₋₆alkyl, and C₁₋₆haloalkyl. In some embodiments or a compound of Formula (I), (Ia), (Ib), or (Ic), R^{1a} is —C(O)NR¹⁸R¹⁹, R¹⁸ is —CH₃, and R¹ is C₂₋₉heteroaryl optionally substituted with one or two groups selected from halogen, C₁₋₆alkyl, and C₁₋₆haloalkyl.

Any combination of the groups described above for the various variables is contemplated herein. Throughout the specification, groups and substituents thereof are chosen by one skilled in the field to provide stable moieties and compounds

In some embodiments provided herein is a compound having the structure of Formula (II), or a pharmaceutically acceptable salt, solvate, or stereoisomer thereof:

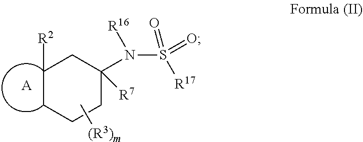

wherein:

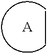

is

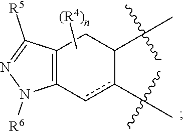

[symbol] is a single bond or a double bond;
R² is hydrogen, halogen, alkyl, alkenyl, —CN, —OR⁸, —NR⁸R⁹, cycloalkyl, heterocycloalkyl, aryl, heteroaryl, —C(O)R¹¹, —C(O)OR⁸, —OC(O)R¹¹, —C(O)NR⁸R⁹, —NR⁸C(O)R¹¹, —NR⁸C(O)OR⁹,—NR¹⁰C(O)NR⁸R⁹, —OC(O)NR⁸R⁹, —S(O)₂R¹¹,—S(O)R¹¹, —SR⁸, —S(O)₂NR⁸R⁹, —NRS(O)₂R¹¹, or —NR¹⁰S(O)₂NR⁸R⁹, wherein alkyl, alkenyl, cycloalkyl, heterocycloalkyl, aryl, and heteroaryl are optionally substituted with one, two, or three R^{12b};
each R³ and each R⁴ is independently halogen or alkyl;
R⁵ is hydrogen, alkyl, or haloalkyl;
R⁶ is aryl, heteroaryl, cycloalkyl, or heterocycloalkyl, wherein aryl, heteroaryl, cycloalkyl, and heterocycloalkyl are optionally substituted with one, two, or three R^{12c};
R⁷ is hydrogen, halogen, —CN, alkyl, haloalkyl, heteroalkyl, alkenyl, —OR⁸, —NR⁸R⁹, cycloalkyl, or heterocycloalkyl;
each R⁸ and each R⁹ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
or R⁸ and R⁹ are taken together with the atom to which they are attached to form a heterocycloalkyl optionally substituted with one, two, or three R^{12a};
R¹⁰ is hydrogen or alkyl;
R¹¹ is alkyl, cycloalkyl, heterocycloalkyl, aryl, or heteroaryl, whel, aryl, and heteroaryl are optionally substituted with one, two, or three R^{12e};
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12f}.
each R^{12b}, R^{12c}, R^{12d}, R^{12e}, and R^{12f} is independently selected from halogen, —CN, alkyl, haloalkyl, —OR¹³, -alkyl-OR^{1,3}—NR¹³R¹⁴, -alkyl-NR¹³R¹⁴, cycloalkyl, -alkyl-cycloalkyl, heterocycloalkyl, aryl, heteroaryl, —C(O)R¹¹,—C(O)OR¹³, —C(O)NR¹³R¹⁴, —S(O)₂R¹⁵, —SR¹³, and —S(O)₂NR¹³R¹⁴; wherein cycloalkyl, heterocycloalkyl, aryl, and heteroaryl are optionally substituted with one, two, or three groups selected from halogen, alkyl, and haloalkyl;
each R¹³ and each R¹⁴ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
or R¹³ and R¹⁴ are taken together with the atom to which they are attached to form a heterocycloalkyl optionally substituted with one, two, or three groups selected from halogen, alkyl, and haloalkyl;
each R¹⁵ is independently alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
m is 0, 1, 2, 3, or 4; and
n is 0, 1, 2, or 3.

In other embodiments provided herein is a compound having the structure of Formula (II), or a pharmaceutically acceptable salt, solvate, or stereoisomer thereof:

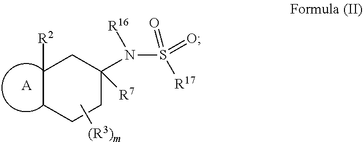

wherein:

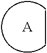

is

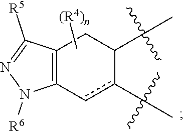

[symbol] is a single bond or a double bond;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀ aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀ aryl, and C₂₋₉heteroaryl are optionally substituted with one, two, or three R^{12b};
each R³ and each R⁴ is independently halogen or C₁₋₆alkyl;
R⁵ is hydrogen, C₁₋₆alkyl, or C₁₋₆haloalkyl;
R⁶ is C₆₋₁₀aryl, C₂₋₉heteroaryl, C₃₋₈cycloalkyl, or C₂₋₉heterocycloalkyl, wherein C₆₋₁₀ aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R⁵, —SR¹³, —S(O)₂NR¹³R¹⁴, —NR¹³S(O)₂R¹⁵, and —NR¹³S(O)₂NR¹³R¹⁴;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f}.
each R^{12b}, R^{2c}, R^{12d}, R^{12e}, and R^{12f} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀aryl, C₂₋₉heteroaryl, —C(O)R⁵, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
m is 0, 1, 2, 3, or 4; and
n is 0, 1, 2, or 3.

In some embodiments,
[symbol] is a single bond or a double bond;
R² is —C(O)R¹¹;
each R³ and each R⁴ is independently halogen or C₁₋₆alkyl;
R⁵ is hydrogen;
R⁶ is C₆₋₁₀aryl optionally substituted with one, two, or three R^{12c};
R⁷ is hydrogen, halogen, or C₁₋₆alkyl;
R¹¹ is C₂₋₉heteroaryl optionally substituted with one, two, or three R^{12e};
each R^{12a}, R^{12b}, R^{12c}, R^{12d}, R^{12e}, R^{12f}, and R^{12g} is independently selected from halogen, C₁₋₆alkyl, C₁₋₆haloalkyl, —OR¹³, C₃₋₈cycloalkyl, or —C₁₋₆alkyl-C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
each R¹⁵ is independently C₁₋₆alkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, or —S(O)₂R¹¹;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f}.
m is 0; and
n is 0.

In some embodiments,
[symbol] is a single bond or a double bond;
R² is —C(O)R¹¹;
each R³ and each R⁴ is independently halogen or C₁₋₆alkyl;
R⁵ is hydrogen;
R⁶ is phenyl substituted with halogen;
R⁷ is hydrogen, halogen, or C₁₋₆alkyl;
R¹¹ is C₂₋₉heteroaryl optionally substituted with one, two, or three R^{12e};
each R^{12e} is independently selected from halogen, C₁₋₆alkyl, C₁₋₆haloalkyl, —OR¹³, or C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, or —C(O)OR¹³;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f};
each R^{12f} is independently selected from halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three groups selected from halogen and C₁₋₆haloalkyl;
m is 0; and
n is 0.

In some embodiments,
[symbol] is a double bond;
R² is —C(O)R¹¹;
each R³ and each R⁴ is independently halogen or C₁₋₆alkyl;
R⁵ is hydrogen;
R⁶ is phenyl substituted with halogen;
R⁷ is hydrogen, halogen, or C₁₋₆alkyl;
R¹¹ is C₂₋₉heteroaryl optionally substituted with one, two, or three R^{12e};
each R^{12e} is independently selected from halogen, C₁₋₆alkyl, C₁₋₆haloalkyl, —OR¹¹, or C₃₋₈cycloalkyl;
each R¹³ is independently hydrogen or C₁₋₆alkyl, wherein C₁₋₆alkyl is optionally substituted with one, two, or three groups selected from halogen, C₁₋₆alkyl, and C₁₋₆haloalkyl;
R¹⁶ is C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, or —C(O)OR¹³;
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f};
each R^{12f} is independently selected from halogen, C₁₋₆alkyl, C₁₋₆haloalkyl, —OR¹³, C₃₋₈cycloalkyl, or —C₁₋₆alkyl-C₃₋₈cycloalkyl; wherein each R¹³ is independently hydrogen or C₁₋₆alkyl, wherein C₁₋₆alkyl is optionally substituted with one, two, or three halogen;
m is 0; and
n is 0.

In some embodiments,
[symbol] is a double bond;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen, halogen, or C₁₋₆alkyl;
R¹¹ is thiazole or pyridine, wherein the thiazole or pyridine is optionally substituted with one, two, or three R^{12e};
each R^{12e} is independently selected from C₁₋₆alkyl, C₁₋₆haloalkyl, or C₃₋₆cycloalkyl;
each R¹³ is independently hydrogen or C₁₋₆alkyl;
R¹⁶ is C₁₋₆alkyl or C₃₋₈cycloalkyl, wherein C₁₋₆alkyl and C₃₋₈cycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, or —C(O)OR¹³;
R¹⁷ is C₄₋₆heterocycloalkyl, phenyl, or C₂₋₉heteroaryl, wherein C₃₋₆alkyl, C₃₋₆cycloalkyl, C₄₋₆heterocycloalkyl, phenyl, and C₂₋₉heteroaryl are optionally substituted with one, two, or three R^{12f};
each R^{12f} is independently selected from halogen, C₁₋₆alkyl, C₁₋₆haloalkyl, C₃₋₈cycloalkyl, or —C₁₋₆alkyl-C₃₋₈cycloalkyl;
m is 0; and
n is 0.

In some embodiments,
[symbol] is a double bond;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen, methyl, or fluoro;
R¹¹ is thiazole or pyridine, wherein the thiazole or pyridine is optionally substituted with one, two, or three R^{12e};
each R^{12e} is independently —CF₃ or cyclopropyl;
R¹⁶ is

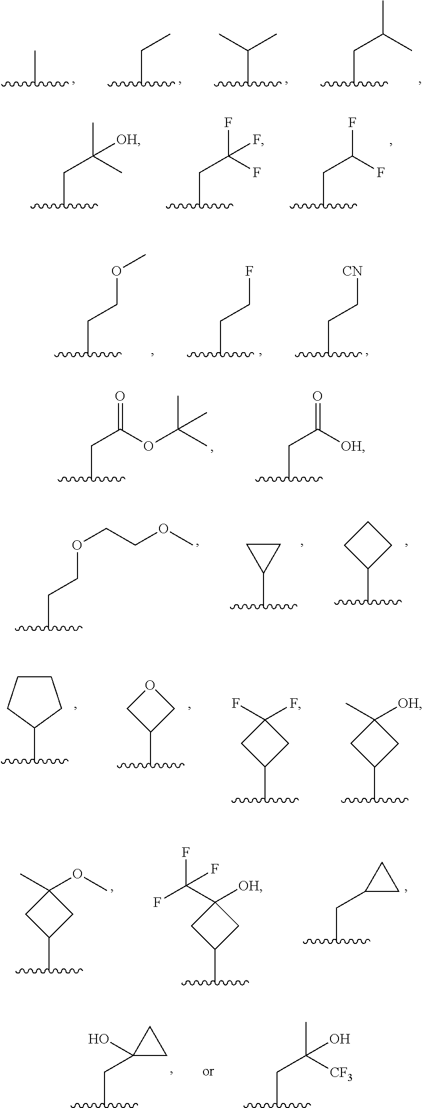

R¹⁷ is tetrahydropyran, phenyl, pyrazole, imidazole, pyridine, or triazole, wherein R¹⁷ is optionally substituted with one, two, or three R^{12f}.
each R^{12f} is independently methyl, ethyl, propyl, iso-propyl, fluoro, chloro, cyclopropyl, methylcyclopropyl, or CHF₂, —CF₃;
m is 0; and
n is 0.

In some embodiments,
[symbol] is a double bond;
R² is —C(O)R¹¹;
each R³ and each R⁴ is independently halogen or C₁₋₆alkyl;
R⁵ is hydrogen;
R⁶ is 4-fluoro-phenyl;
R⁷ is hydrogen;
R¹¹ is thiazole or pyridine, wherein the thiazole or pyridine is optionally substituted with one, two, or three R^{12e};
each R^{12e} is independently —CF₃ or cyclopropyl;
R¹⁶ is methyl, ethyl, cyclopropyl, or methylcyclopropyl;
R¹⁷ is tetrahydropyran, phenyl, pyrazole, pyridine, or triazole, wherein R¹ is optionally substituted with one, two, or three R^{12f};
   each R^{12f} is independently methyl, ethyl, propyl, iso-propyl, fluoro, chloro, methylcyclopropyl, or —CF₃;
m is 0; and
n is 0.

In some embodiments, a compound of Formula (II), or a pharmaceutically acceptable salt, solvate, or stereoisomer thereof, has the structure:

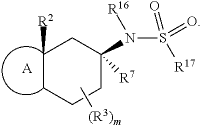

In some embodiments, a compound of Formula (II), or a pharmaceutically acceptable salt, solvate, or stereoisomer thereof, has the structure:

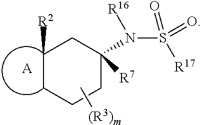

In some embodiments, a compound of Formula (II), or a pharmaceutically acceptable salt, solvate, or stereoisomer thereof, has the structure:

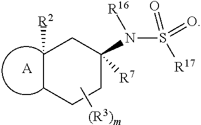

In some embodiments, a compound of Formula (II), or a pharmaceutically acceptable salt, solvate, or stereoisomer thereof, has the structure:

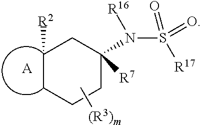

In some embodiments provided herein is a compound having the structure of Formula (IIa), or a pharmaceutically acceptable salt, solvate, or stereoisomer thereof:

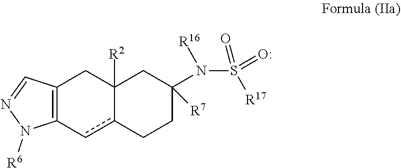

wherein:
[symbol] is a single bond or a double bond;
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12b}, R^{12c}, R^{12d}, R^{12e}, and R^{12f} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹¹, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴; and
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f}.

In some embodiments, a compound of Formula (IIa), or a pharmaceutically acceptable salt, solvate, or stereoisomer thereof, has the structure:

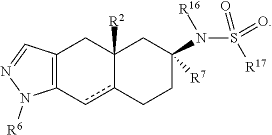

In some embodiments, a compound of Formula (IIa), or a pharmaceutically acceptable salt, solvate, or stereoisomer thereof, has the structure:

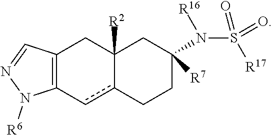

In some embodiments, a compound of Formula (IIa), or a pharmaceutically acceptable salt, solvate, or stereoisomer thereof, has the structure:

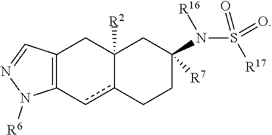

In some embodiments, a compound of Formula (IIa), or a pharmaceutically acceptable salt, solvate, or stereoisomer thereof, has the structure:

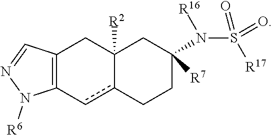

In some embodiments provided herein is a compound having the structure of Formula (IIb), or a pharmaceutically acceptable salt, solvate, or stereoisomer thereof:

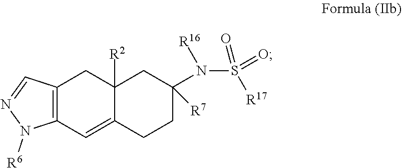

wherein:
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸,—S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R¹²;
or R⁸ and R⁹ are taken together with the atom to which they are attached to form a C₂₋₉heterocycloalkyl optionally substituted with one, two, or three R^{12d};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12b}, R^{12c}, R^{12d}, R^{12e}, and R^{12f} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹¹, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴; and
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12f}.

In some embodiments, a compound of Formula (IIb), or a pharmaceutically acceptable salt, solvate, or stereoisomer thereof, has the structure:

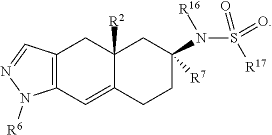

In some embodiments, a compound of Formula (IIb), or a pharmaceutically acceptable salt, solvate, or stereoisomer thereof, has the structure:

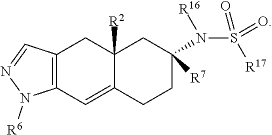

In some embodiments, a compound of Formula (IIb), or a pharmaceutically acceptable salt, solvate, or stereoisomer thereof, has the structure:

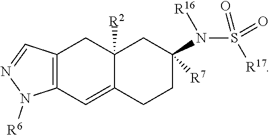

In some embodiments, a compound of Formula (IIb), or a pharmaceutically acceptable salt, solvate, or stereoisomer thereof, has the structure:

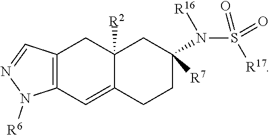

In some embodiments provided herein is a compound having the structure of Formula (IIc), or a pharmaceutically acceptable salt, solvate, or stereoisomer thereof:

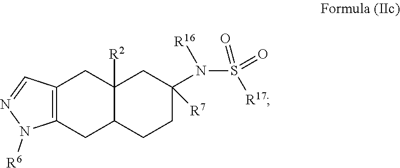

wherein:
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸,—S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl, C₂₋₉heteroaryl, C₃₋₈cycloalkyl, or C₂₋₉heterocycloalkyl, wherein C₆₋₁₀aryl, C₂₋₉heteroaryl, C₃₋₈cycloalkyl, and C₂₋₉heterocycloalkyl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12b}, R^{12c}, R^{12d}, R^{12e}, and R^{12f} is independently selected from halogen, —CN, C₁₋₆alkyl, C₁₋₆haloalkyl, —OR¹³, —C₁₋₆alkyl-OR¹³, —NR¹³R¹⁴, —C₁₋₆alkyl-NR¹³R¹⁴, C₃₋₈cycloalkyl, —C₁₋₆alkyl-C₃₋₈cycloalkyl, C₂₋₉heterocycloalkyl, C₆₋₁₀ aryl, C₂₋₉heteroaryl, —C(O)R¹¹, —C(O)OR¹³, —C(O)NR¹³R¹⁴, —S(O)₂R¹⁵, —SR¹³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹⁵ is independently C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³,—NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴; and
R¹⁷ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12f}.

In some embodiments, a compound of Formula (IIc), or a pharmaceutically acceptable salt, solvate, or stereoisomer thereof, has the structure:

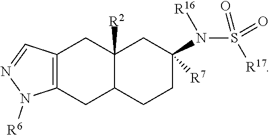

In some embodiments, a compound of Formula (IIc), or a pharmaceutically acceptable salt, solvate, or stereoisomer thereof, has the structure:

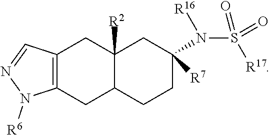

In some embodiments, a compound of Formula (IIc), or a pharmaceutically acceptable salt, solvate, or stereoisomer thereof, has the structure:

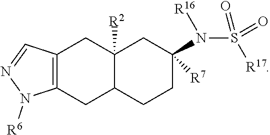

In some embodiments, a compound of Formula (IIc), or a pharmaceutically acceptable salt, solvate, or stereoisomer thereof, has the structure:

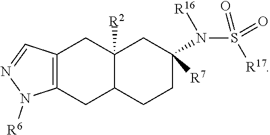

In some embodiments or a compound of Formula (II), (IIa), (IIb), or (IIc), R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹, —NR¹³R¹⁴, —C(O)R⁵, —C(O)OR¹, —C(O)NR¹³R¹⁴, —NR¹³C(O)R⁵, —NR¹³C(O)OR¹³, —NR¹³C(O)NR¹³R¹⁴, —S(O)₂R¹⁵, —S(O)R⁵, —SR 1, —S(O)₂NR¹³R¹⁴,—NR¹³S(O)₂R¹⁵, and —NR¹³S(O)₂NR³R⁴. In some embodiments or a compound of Formula (II), (IIa), (IIb), or (IIc), R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR³, —C(O)OR¹³, and —S(O)₂R¹⁵. In some embodiments or a compound of Formula (II), (IIa), (IIb), or (IIc), R¹⁶ is hydrogen, C₁₋₆alkyl, C₃₋₈cycloalkyl, or C₂₋₉heterocycloalkyl, wherein C₁₋₆alkyl, C₃₋₈cycloalkyl, and C₂₋₉heterocycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR³, —C(O)OR¹³, and —S(O)₂R¹⁵, R¹³ is hydrogen or C₁₋₆alkyl, and R¹¹ is C₁₋₆alkyl. In some embodiments or a compound of Formula (II), (IIa), (IIb), or (IIc), R¹⁶ is

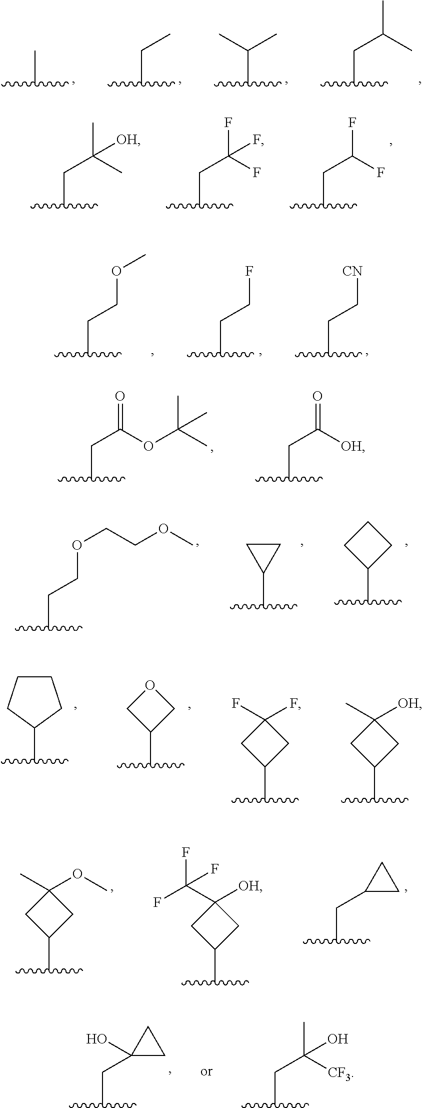

In some embodiments or a compound of Formula (II), (IIa), (IIb), or (IIc), R¹⁶ is unsubstituted C₁₋₆alkyl. In some embodiments or a compound of Formula (II), (IIa), (IIb), or (IIc), R¹⁶ is —CH₃. In some embodiments or a compound of Formula (II), (IIa), (IIb), or (IIc), R¹⁶ is unsubstituted C₃₋₈cycloalkyl. In some embodiments or a compound of Formula (II), (IIa), (IIb), or (IIc), R¹⁶ is unsubstituted cyclopropyl.

In some embodiments or a compound of Formula (II), (IIa), (IIb), or (IIc), R⁷ is C₆₋₁₀aryl or C₂₋₉heteroaryl, wherein C₆₋₁₀aryl and C₂₋₉heteroaryl are optionally substituted with one, two, or three R^{12f}. In some embodiments or a compound of Formula (II), (IIa), (IIb), or (IIc), R⁷ is phenyl optionally substituted with one, two, or three R^{2f}. In some embodiments or a compound of Formula (II), (IIa), (IIb), or (IIc), R is phenyl optionally substituted with one, two, or three groups selected from halogen, C₁₋₆alkyl, and C₁₋₆haloalkyl. In some embodiments or a compound of Formula (II), (IIa), (IIb), or (IIc), R⁷ is C₂₋₉heteroaryl optionally substituted with one, two, or three R^{12f}. In some embodiments or a compound of Formula (II), (IIa), (IIb), or (IIc), R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three R^{12f}. In some embodiments or a compound of Formula (II), (IIa), (IIb), or (IIc), R¹⁷ is selected from pyrazole, thiazole, thiadiazole, oxazole, isoxazole, imidazole, triazole, and pyridine, wherein pyrazole, thiazole, thiadiazole, oxazole, isoxazole, imidazole, triazole, and pyridine are optionally substituted with one, two, or three groups selected from halogen, C₁₋₆alkyl, and C₁₋₆haloalkyl. In some embodiments or a compound of Formula (II), (IIa), (IIb), or (IIc), R¹⁷ is selected from pyrazole, triazole, and pyridine, wherein pyrazole, triazole, and pyridine are optionally substituted with one, two, or three groups selected from halogen, C₁₋₆alkyl, and C₁₋₆haloalkyl.

Any combination of the groups described above for the various variables is contemplated herein. Throughout the specification, groups and substituents thereof are chosen by one skilled in the field to provide stable moieties and compounds.

In some embodiments provided herein is a compound having the structure of Formula (III), or a pharmaceutically acceptable salt, solvate, or stereoisomer thereof:

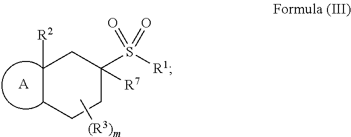

wherein:

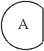

is

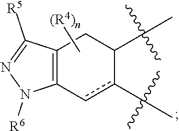

[symbol] is a single bond or a double bond;
R¹ is aryl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
R² is hydrogen, halogen, alkyl, alkenyl, —CN, —OR⁸, —NR⁸R⁹, cycloalkyl, heterocycloalkyl, aryl, heteroaryl, —C(O)R¹¹,—C(O)OR⁸, —OC(O)R¹¹,—C(O)NR⁸R⁹, —NR⁸C(O)R¹¹, —NR⁸C(O)OR⁹,—NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein alkyl, alkenyl, cycloalkyl, heterocycloalkyl, aryl, and heteroaryl are optionally substituted with one, two, or three R^{12b};
each R³ and each R⁴ is independently halogen or alkyl;
R⁵ is hydrogen, alkyl, or haloalkyl;
R⁶ is aryl or heteroaryl, wherein aryl and heteroaryl are optionally substituted with one, two, or three R^{12c};
R⁷ is hydrogen, halogen, —CN, alkyl, haloalkyl, heteroalkyl, alkenyl, —OR⁸, —NR⁸R⁹, cycloalkyl, or heterocycloalkyl;
each R⁸ and each R⁹ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
or R⁸ and R⁹ are taken together with the atom to which they are attached to form a heterocycloalkyl optionally substituted with one, two, or three R^{12a};
R¹⁰ is hydrogen or alkyl;
R¹¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12e};
each R^{12a}, R^{12b}, R^{12c}, R^{12d}, and R^{12e} is independently selected from halogen, —CN, alkyl, haloalkyl, —OR¹³, -alkyl-OR¹³, —NR¹³R¹⁴, -alkyl-NR¹³R¹⁴, cycloalkyl, heterocycloalkyl, aryl, heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein cycloalkyl, heterocycloalkyl, aryl, and heteroaryl are optionally substituted with one, two, or three groups selected from halogen, alkyl, and haloalkyl;
each R¹³ and each R¹⁴ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
   or R¹³ and R¹⁴ are taken together with the atom to which they are attached to form a heterocycloalkyl optionally substituted with one, two, or three groups selected from halogen, alkyl, and haloalkyl;
each R¹⁵ is independently alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
m is 0, 1, 2, 3, or 4; and
n is 0, 1, 2, or 3.

In other embodiments provided herein is a compound having the structure of Formula (III), or a pharmaceutically acceptable salt, solvate, or stereoisomer thereof:

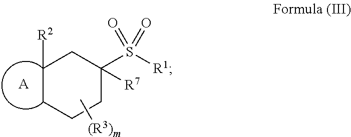

wherein:

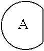

is

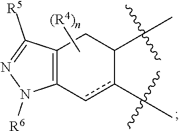

[symbol] is a single bond or a double bond;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
each R³ and each R⁴ is independently halogen or C₁₋₆alkyl;
R⁵ is hydrogen, C₁₋₆alkyl, or C₁₋₆haloalkyl;
R⁶ is C₆₋₁₀aryl or C₂₋₉heteroaryl, wherein C₆₋₁₀ aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, and R^{12e} is independently selected from halogen, —CN, C₁₋₆alkyl, C₁₋₆haloalkyl, —OR³, —C₁₋₆alkyl-OR³, —NR³R⁴, —C₁₋₆alkyl-NR³R⁴, C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
   or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl;
each R¹¹ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
m is 0, 1, 2, 3, or 4; and
n is 0, 1, 2, or 3.

In some embodiments, a compound of Formula (III), or a pharmaceutically acceptable salt, solvate, or stereoisomer thereof, has the structure:

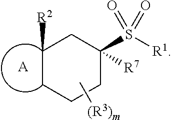

In some embodiments, a compound of Formula (III), or a pharmaceutically acceptable salt, solvate, or stereoisomer thereof, has the structure:

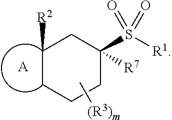

In some embodiments, a compound of Formula (III), or a pharmaceutically acceptable salt, solvate, or stereoisomer thereof, has the structure:

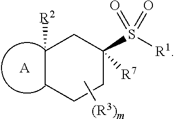

In some embodiments, a compound of Formula (III), or a pharmaceutically acceptable salt, solvate, or stereoisomer thereof, has the structure:

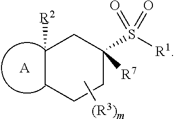

In some embodiments provided herein is a compound having the structure of Formula (IIa), or a pharmaceutically acceptable salt, solvate, or stereoisomer thereof:

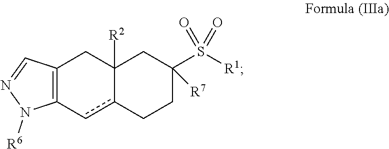

wherein:
[symbol] is a single bond or a double bond;
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, [symbol] C₃₋₈cycloalkyl, C₂₋₉heterocycloalkyl, C₆₋₁₀aryl, and C₂₋₉heteroaryl are optionally substituted with one, two, or three R^{12a};
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸,—S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀ aryl or C₂₋₉heteroaryl, wherein C₆₋₁₀ aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, and R^{12e} is independently selected from halogen, —CN, C₁₋₆alkyl, C₁₋₆haloalkyl, —OR³, —C₁₋₆alkyl-OR¹³, —NR¹³R¹⁴, —C₁₋₆alkyl-NR¹³R¹⁴, C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
   or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl; and
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl.

In some embodiments, a compound of Formula (IIIa), or a pharmaceutically acceptable salt, solvate, or stereoisomer thereof, has the structure:

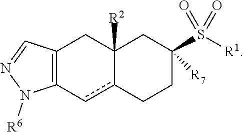

In some embodiments, a compound of Formula (IIIa), or a pharmaceutically acceptable salt, solvate, or stereoisomer thereof, has the structure:

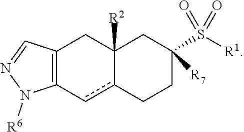

In some embodiments, a compound of Formula (IIIa), or a pharmaceutically acceptable salt, solvate, or stereoisomer thereof, has the structure:

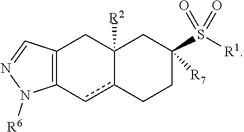

In some embodiments, a compound of Formula (IIIa), or a pharmaceutically acceptable salt, solvate, or stereoisomer thereof, has the structure:

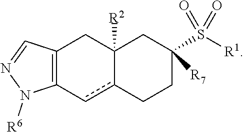

In some embodiments provided herein is a compound having the structure of Formula (IIb), or a pharmaceutically acceptable salt, solvate, or stereoisomer thereof:

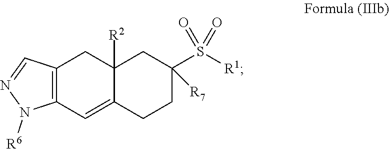

wherein:
R¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
R² is hydrogen, halogen, C₁₋₆alkyl, C₂₋₆alkenyl, —CN, —OR⁸, —NR⁸R⁹, C₃₋₈cycloalkyl, C₂₋₉heterocycloalkyl, C₆₋₁₀aryl, C₂₋₉heteroaryl, —C(O)R¹¹, —C(O)OR⁸, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₋₁₀aryl, and C₂₋₉heteroaryl are optionally substituted with one, two, or three R^{12b};
R⁶ is C₆₋₁₀aryl or C₂₋₉heteroaryl, wherein C₆₋₁₀aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl;
each R⁸ and each R⁹ is independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12e};
each R^{12a}, R^{12b}, R^{12c}, R^{12d}, and R^{12e} is independently selected from halogen, —CN, C₁₋₆alkyl, C₁₋₆haloalkyl, —OR³, —C₁₋₆alkyl-OR³, —NR³R⁴, —C₁₋₆alkyl-NR³R⁴, C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl; and
each R¹⁵ is independently C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl.

In some embodiments, a compound of Formula (IIIb), or a pharmaceutically acceptable salt, solvate, or stereoisomer thereof, has the structure:

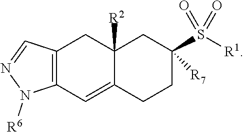

In some embodiments, a compound of Formula (IIIb), or a pharmaceutically acceptable salt, solvate, or stereoisomer thereof, has the structure:

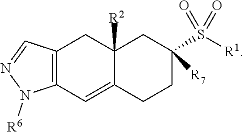

In some embodiments, a compound of Formula (IIIb), or a pharmaceutically acceptable salt, solvate, or stereoisomer thereof, has the structure:

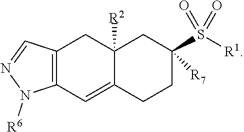

In some embodiments, a compound of Formula (IIIb), or a pharmaceutically acceptable salt, solvate, or stereoisomer thereof, has the structure:

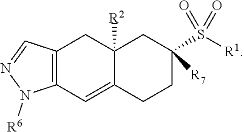

In some embodiments provided herein is a compound having the structure of Formula (IIIc), or a pharmaceutically acceptable salt, solvate, or stereoisomer thereof:

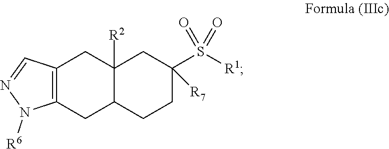

wherein:
R¹ is C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀aryl, and C₂₋₉heteroaryl are optionally substituted with one, two, or three R^{12a};
R² is hydrogen, halogen, C₁₋₆alkyl, C₂₋₆alkenyl, —CN, —OR^{B}, —NR⁸R⁹, C₃₋₈cycloalkyl, C₂₋₉heterocycloalkyl, C₆₋₁₀aryl, C₂₋₉heteroaryl, —C(O)R¹¹, —C(O)OR^{B}, —OC(O)R¹¹, —C(O)NR⁸R⁹, —NR⁸C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NRS(O)₂R¹¹, or —NR¹⁰S(O)₂NR⁸R⁹, wherein C₁₋₆alkyl, C₂₋₆alkenyl, C₃₋₈cycloalkyl, C₂₋₉heterocycloalkyl, C₆₁aryl, and C₂₋₉heteroaryl are optionally substituted with one, two, or three R^{12b};
R⁶ is C₆₋₁₀aryl or C₂₋₉heteroaryl, wherein C₆₋₁₀ aryl and C₂₋₉heteroaryl are optionally substituted with one, two, or three R^{12c};
R⁷ is hydrogen, halogen, —CN, C₁₋₆alkyl, C₁₋₆haloalkyl, C₁₋₆heteroalkyl, C₂₋₆alkenyl, —OR⁸, —NR⁸R⁹, C₃₋₈cycloalkyl, or C₂₋₉heterocycloalkyl
R⁸ and R⁹ are each independently hydrogen, C₁₋₆alkyl, C₃₋₈cycloalkyl, C₂₋₉heterocycloalkyl, C₆₋₁₀aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12a};
or R⁸ and R⁹ are taken together with the atom to which they are attached to form a C₂₋₉heterocycloalkyl optionally substituted with one, two, or three R^{12a};
R¹⁰ is hydrogen or C₁₋₆alkyl;
R¹¹ is C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three R^{12e};
each R^{12a}, R^{12b}, R^{12c}, R^{12d}, and R^{12e} is independently selected from halogen, —CN, C₁₋₆alkyl, C₁₋₆haloalkyl, —OR³, —C₁₋₆alkyl-OR³, —NR³R⁴, —C₁₋₆alkyl-NR³R⁴, C₃₋₈cycloalkyl, C₂₋₉heterocycloalkyl, C₆₋₁₀ aryl, C₂₋₉heteroaryl, —C(O)R¹⁵, —C(O)OR¹³, —C(O)NR¹³R¹⁴, —S(O)₂R¹⁵, —SR³, and —S(O)₂NR¹³R¹⁴; wherein C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
each R¹³ and each R¹⁴ are independently hydrogen, C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl;
or R¹³ and R¹⁴ are taken together with the atom to which they are attached to form a C₂₋₉heterocycloalkyl optionally substituted with one, two, or three groups selected from halogen, C₁₋₆alkyl, and C₁₋₆haloalkyl; and
each R¹⁵ is independently C₁₋₆alkyl, C₃₋₈cycloalkyl, C₂₋₉heterocycloalkyl, C₆₋₁₀ aryl, or C₂₋₉heteroaryl, wherein C₁₋₆alkyl, C₃₋₈cycloalkyl, C₂₋₉heterocycloalkyl, C₆₋₁₀ aryl, and C₂₋₉heteroaryl are optionally substituted with one, two, or three groups selected from halogen, C₁₋₆alkyl, and C₁₋₆haloalkyl.

In some embodiments, a compound of Formula (IIIc), or a pharmaceutically acceptable salt, solvate, or stereoisomer thereof, has the structure:

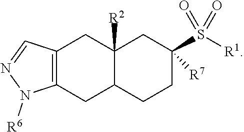

In some embodiments, a compound of Formula (IIIc), or a pharmaceutically acceptable salt, solvate, or stereoisomer thereof, has the structure:

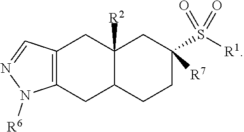

In some embodiments, a compound of Formula (IIIc), or a pharmaceutically acceptable salt, solvate, or stereoisomer thereof, has the structure:

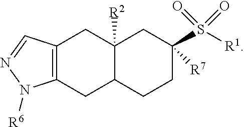

In some embodiments, a compound of Formula (IIIc), or a pharmaceutically acceptable salt, solvate, or stereoisomer thereof, has the structure:

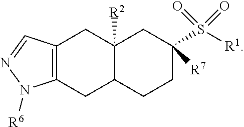

In some embodiments is a compound of Formula (III), (IIIa), (IIIb), or (IIc), or a pharmaceutically acceptable salt, solvate, or stereoisomer thereof, wherein R¹ is C₆₋₁₀aryl optionally substituted with one, two, or three R^{12a}. In some embodiments is a compound of Formula (III), (IIIa), (IIIb), or (IIIc), or a pharmaceutically acceptable salt, solvate, or stereoisomer thereof, wherein R¹ is phenyl optionally substituted with one, two, or three R^{12a}. In some embodiments is a compound of Formula (III), (IIIa), (IIIb), or (IIIc), or a pharmaceutically acceptable salt, solvate, or stereoisomer thereof, wherein R¹ is unsubstituted phenyl. In some embodiments is a compound of Formula (III), (IIIa), (IIb), or (IIc), or a pharmaceutically acceptable salt, solvate, or stereoisomer thereof, wherein R¹ is phenyl substituted with one, two, or three groups selected from halogen, —CN, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is phenyl substituted with one, two, or three groups selected from halogen, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is phenyl substituted with one, two, or three halogens. In some embodiments is a compound of Formula (III), (IIIa), (IIIb), or (IIIc), or a pharmaceutically acceptable salt, solvate, or stereoisomer thereof, wherein R¹ is phenyl substituted with two halogens. In some embodiments is a compound of Formula (III), (IIIa), (IIIb), or (IIIc), or a pharmaceutically acceptable salt, solvate, or stereoisomer thereof, wherein R¹ is phenyl substituted with one halogen. In some embodiments is a compound of Formula (III), (IIIa), (IIIb), or (IIIc), or a pharmaceutically acceptable salt, solvate, or stereoisomer thereof, wherein R¹ is phenyl substituted with one C₁₋₆alkyl. In some embodiments is a compound of Formula (III), (IIIa), (IIIb), or (IIIc), or a pharmaceutically acceptable salt, solvate, or stereoisomer thereof, wherein R¹ is phenyl substituted with one —CH₃. In some embodiments is a compound of Formula (III), (IIIa), (IIIb), or (IIIc), or a pharmaceutically acceptable salt, solvate, or stereoisomer thereof, wherein R¹ is phenyl substituted with one C₁₋₆haloalkyl. In some embodiments is a compound of Formula (III), (IIIa), (IIIb), or (IIIc), or a pharmaceutically acceptable salt, solvate, or stereoisomer thereof, wherein R¹ is phenyl substituted with one —CF₃. In some embodiments is a compound of Formula (III), (IIIa), (IIIb), or (IIIc), or a pharmaceutically acceptable salt, solvate, or stereoisomer thereof, wherein R¹ is phenyl substituted with one —CN.

In some embodiments is a compound of Formula (III), (IIIa), (IIIb), or (IIIc), or a pharmaceutically acceptable salt, solvate, or stereoisomer thereof, wherein R¹ is C₂₋₉heteroaryl optionally substituted with one, two, or three R^{12a}. In some embodiments is a compound of Formula (III), (IIIa), (IIIb), or (IIc), or a pharmaceutically acceptable salt, solvate, or stereoisomer thereof, wherein R¹ is selected from pyrazole, thiazole, oxazole, isoxazole, imidazole, triazole, and pyridine, wherein pyrazole, thiazole, oxazole, isoxazole, imidazole, triazole, and pyridine are optionally substituted with one, two, or three R^{12a}. In some embodiments is a compound of Formula (III), (IIIa), (IIIb), or (IIIc), or a pharmaceutically acceptable salt, solvate, or stereoisomer thereof, wherein R¹ is selected from pyrazole, triazole, and pyridine, wherein pyrazole, triazole, and pyridine are optionally substituted with one, two, or three R¹². In some embodiments is a compound of Formula (III), (IIIa), (IIIb), or (IIIc), or a pharmaceutically acceptable salt, solvate, or stereoisomer thereof, wherein R¹ is unsubstituted pyrazole, triazole, or pyridine. In some embodiments is a compound of Formula (III), (IIIa), (IIIb), or (IIIc), or a pharmaceutically acceptable salt, solvate, or stereoisomer thereof, wherein R¹ is pyrazole substituted with one or two groups selected from halogen, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is pyrazole substituted with one C₁₋₆alkyl. In some embodiments is a compound of Formula (III), (IIIa), (IIIb), or (IIIc), or a pharmaceutically acceptable salt, solvate, or stereoisomer thereof, wherein R¹ is pyrazole substituted with one —CH₃. In some embodiments is a compound of Formula (III), (IIIa), (IIIb), or (IIIc), or a pharmaceutically acceptable salt, solvate, or stereoisomer thereof, wherein R¹ is triazole substituted with one or two groups selected from halogen, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is triazole substituted with one C₁₋₆alkyl. In some embodiments is a compound of Formula (III), (IIIa), (IIIb), or (IIIc), or a pharmaceutically acceptable salt, solvate, or stereoisomer thereof, wherein R¹ is triazole substituted with one —CH₃. In some embodiments is a compound of Formula (III), (IIIa), (IIIb), or (IIc), or a pharmaceutically acceptable salt, solvate, or stereoisomer thereof, wherein R¹ is pyridine substituted with one or two groups selected from halogen, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is pyridine substituted with one or two halogens. In some embodiments is a compound of Formula (III), (IIIa), (IIIb), or (IIIc), or a pharmaceutically acceptable salt, solvate, or stereoisomer thereof, wherein R¹ is pyridine substituted with one halogen. In some embodiments is a compound of Formula (III), (IIIa), (IIIb), or (IIIc), or a pharmaceutically acceptable salt, solvate, or stereoisomer thereof, wherein R¹ is pyridine substituted with one C₁₋₆alkyl. In some embodiments is a compound of Formula (III), (IIIa), (IIIb), or (IIIc), or a pharmaceutically acceptable salt, solvate, or stereoisomer thereof, wherein R¹ is pyridine substituted with one —CH₃. In some embodiments is a compound of Formula (III), (IIIa), (IIIb), or (IIIc), or a pharmaceutically acceptable salt, solvate, or stereoisomer thereof, wherein R¹ is pyridine substituted with one C₁₋₆haloalkyl. In some embodiments is a compound of Formula (III), (IIIa), (IIIb), or (IIIc), or a pharmaceutically acceptable salt, solvate, or stereoisomer thereof, wherein R¹ is pyridine substituted with one —CF₃.

In some embodiments is a compound of Formula (III), (IIIa), (IIIb), or (IIIc), or a pharmaceutically acceptable salt, solvate, or stereoisomer thereof, wherein R¹ is C₃₋₈cycloalkyl optionally substituted with one, two, or three R^{12a}. In some embodiments is a compound of Formula (III), (IIIa), (IIIb), or (IIc), or a pharmaceutically acceptable salt, solvate, or stereoisomer thereof, wherein R¹ is selected from cyclobutyl, cyclopentyl and cyclohexyl, wherein cyclobutyl, cyclopentyl and cyclohexyl, are optionally substituted with one, two, or three R^{12a}. In some embodiments is a compound of Formula (III), (IIIa), (IIIb), or (IIc), or a pharmaceutically acceptable salt, solvate, or stereoisomer thereof, wherein R¹ is unsubstituted cyclobutyl. In some embodiments is a compound of Formula (III), (IIIa), (IIIb), or (IIIc), or a pharmaceutically acceptable salt, solvate, or stereoisomer thereof, wherein R¹ is unsubstituted cyclopentyl. In some embodiments is a compound of Formula (III), (IIIa), (IIIb), or (IIIc), or a pharmaceutically acceptable salt, solvate, or stereoisomer thereof, wherein R¹ is unsubstituted cyclohexyl.

In some embodiments is a compound of Formula (III), (IIIa), (IIIb), or (IIc), or a pharmaceutically acceptable salt, solvate, or stereoisomer thereof, wherein R¹ is C₁₋₆alkyl optionally substituted with one, two, or three R^{12a}. In some embodiments is a compound of Formula (III), (IIIa), (IIIb), or (IIc), or a pharmaceutically acceptable salt, solvate, or stereoisomer thereof, wherein R¹ is C₁₋₆alkyl substituted with phenyl, wherein phenyl is optionally substituted with one, two, or three groups selected from halogen, C₁₋₆alkyl, and C₁₋₆haloalkyl. In some embodiments is a compound of Formula (III), (IIIa), (IIIb), or (IIIc), or a pharmaceutically acceptable salt, solvate, or stereoisomer thereof, wherein R¹ is C₁₋₆alkyl substituted with phenyl, wherein phenyl is unsubstituted. In some embodiments is a compound of Formula (III), (IIIa), (IIIb), or (IIIc), or a pharmaceutically acceptable salt, solvate, or stereoisomer thereof, wherein R¹ is C₁₋₆alkyl substituted with phenyl, wherein phenyl is substituted with one, two, or three groups selected from halogen, C₁₋₆alkyl, and C₁₋₆haloalkyl.

In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₆alkyl, C₂₋₆alkenyl, C₃₋₈cycloalkyl, or —C(O)R¹¹, wherein C₁₋₆alkyl, C₂₋₆alkenyl, and C₃₋₈cycloalkyl are optionally substituted with one, two, or three R^{12b}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₁₋₆ alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three R^{12b}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₁₋₆alkyl, C₂₋₆alkenyl, or C₃₋₈cycloalkyl, wherein C₁₋₆alkyl, C₂₋₆alkenyl, and C₃₋₈cycloalkyl are optionally substituted with one, two, or three groups selected from halogen, C₁₋₆alkyl, C₁₋₆haloalkyl, —OR¹,—NR¹³R¹⁴, and C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₁₋₆ alkyl optionally substituted with one, two, or three groups selected from halogen, —OR¹³, —NR¹³R¹⁴, and C₂₋₉heteroaryl. In some embodiments is a compound of Formula (I), (Ia)-(Ic), (II), (IIa)-(IIc), (III), or (IIIa)-(IIc), or a pharmaceutically acceptable salt, solvate, or stereoisomer thereof, wherein R² is C₁₋₃alkyl substituted with one, two, or three groups selected from halogen, —OR¹³, —NR¹³R¹⁴, and C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₁₋₃alkyl substituted with one, two, or three groups selected from halogen, —OH, and C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₁₋₃alkyl substituted with one, two, or three groups selected from halogen, —OH, and pyridine. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₂₋₆alkenyl optionally substituted with one, two, or three groups selected from halogen, —OR³, —NR¹³R¹⁴, and C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₂₋₆alkenyl substituted with one C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₂₋₆alkenyl substituted with one pyridine. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₃₋₈cycloalkyl optionally substituted with one, two, or three groups selected from halogen, —OR¹³, —NR¹³R¹⁴, and C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₃₋₈cycloalkyl substituted with one C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is C₃₋₈cycloalkyl substituted with one pyridine. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is cyclopropyl substituted with one C₂₋₉heteroaryl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is cyclopropyl substituted with one pyridine.

In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is C₆₋₁₀aryl or C₂₋₉heteroaryl, wherein C₆₋₁₀ aryl and C₂₋₉heteroaryl are optionally substituted with one, two, or three R^{12e}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is C₂₋₉heteroaryl optionally substituted with one, two, or three R¹². In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is C₂₋₉heteroaryl optionally substituted with one or two R^{12e}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is C₂₋₉heteroaryl optionally substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is selected from thiazole and pyridine, wherein thiazole and pyridine are optionally substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is thiazole optionally substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is unsubstituted thiazole. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is thiazole substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine optionally substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is unsubstituted pyridine. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one halogen. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one C₁₋₆alkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one —CH₃. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R² is —C(O)R¹¹ and R¹¹ is pyridine substituted with one —CF₃.

In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R is C₆₋₁₀aryl, C₂₋₉heteroaryl, or C₂₋₉heterocycloalkyl, wherein C₆₋₁₀ aryl, C₂₋₉heteroaryl, and C₂₋₉heterocycloalkyl are optionally substituted with one, two, or three R¹². In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is C₆₋₁₀aryl or C₂₋₉heteroaryl, wherein C₆₋₁₀ aryl and C₂₋₉heteroaryl are optionally substituted with one, two, or three R^{12c}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is C₆₋₁₀ aryl optionally substituted with one, two, or three R^{12c}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl optionally substituted with one, two, or three R^{12c}. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is unsubstituted phenyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl optionally substituted with one, two, or three groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one or two groups selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one group selected from halogen, C₁₋₆alkyl, and C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one halogen. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one F. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one C₁. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one C₁₋₆alkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one —CH₃. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R is phenyl substituted with one C₁₋₆haloalkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁶ is phenyl substituted with one —CF₃.

In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is hydrogen, halogen, or C₁₋₆alkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is hydrogen or C₁₋₆alkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is hydrogen. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is C₁₋₆alkyl. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is —CH₃. In some embodiments is a compound of Formula (I), (Ia)-(Ic), (II), (IIa)-(IIc), (III), or (IIIa)-(IIIc), or a pharmaceutically acceptable salt, solvate, or stereoisomer thereof, wherein R⁷ is fluoro.

In some embodiments is a compound of Formula (I), (II), or (III), or a pharmaceutically acceptable salt or solvate thereof, wherein R⁵ is hydrogen. In some embodiments is a compound of Formula (I), (II), or (III), or a pharmaceutically acceptable salt or solvate thereof, wherein R is C₁₋₆alkyl. In some embodiments is a compound of Formula (I), (II), or (III), or a pharmaceutically acceptable salt or solvate thereof, wherein R is —CH₃. In some embodiments is a compound of Formula (I), (II), or (III), or a pharmaceutically acceptable salt or solvate thereof, wherein R⁵ is C₁₋₆haloalkyl. In some embodiments is a compound of Formula (I), (II), or (III), or a pharmaceutically acceptable salt or solvate thereof, wherein R⁵ is —CF₃.

In some embodiments is a compound of Formula (I), (II), or (III), or a pharmaceutically acceptable salt or solvate thereof, wherein m is 0. In some embodiments is a compound of Formula (I), (II), or (III), or a pharmaceutically acceptable salt or solvate thereof, wherein m is 1. In some embodiments is a compound of Formula (I), (II), or (III), or a pharmaceutically acceptable salt or solvate thereof, wherein m is 2. In some embodiments is a compound of Formula (I), (II), or (III), or a pharmaceutically acceptable salt or solvate thereof, wherein m is 3. In some embodiments is a compound of Formula (I), (II), or (III), or a pharmaceutically acceptable salt or solvate thereof, wherein m is 4. In some embodiments is a compound of Formula (I), (II), or (III), or a pharmaceutically acceptable salt or solvate thereof, wherein n is 0. In some embodiments is a compound of Formula (I), (II), or (III), or a pharmaceutically acceptable salt or solvate thereof, wherein n is 1. In some embodiments is a compound of Formula (I), (II), or (III), or a pharmaceutically acceptable salt or solvate thereof, wherein m is 2. In some embodiments is a compound of Formula (I), (II), or (III), or a pharmaceutically acceptable salt or solvate thereof, wherein n is 3. In some embodiments is a compound of Formula (I), (II), or (III), or a pharmaceutically acceptable salt or solvate thereof, wherein m is 0 and n is 0.

In some embodiments is a compound of Formula (I), (II), or (III), or a pharmaceutically acceptable salt or solvate thereof, wherein R³ is halogen. In some embodiments is a compound of Formula (I), (II), or (III), or a pharmaceutically acceptable salt or solvate thereof, wherein R³ is C₁₋₆alkyl.

In some embodiments is a compound of Formula (I), (II), or (III), or a pharmaceutically acceptable salt or solvate thereof, wherein R⁴ is halogen. In some embodiments is a compound of Formula (I), (II), or (III), or a pharmaceutically acceptable salt or solvate thereof, wherein R is C₁₋₆alkyl.

In some embodiments is a compound of Formula (I), (II), or (III), or a pharmaceutically acceptable salt or solvate thereof, wherein [symbol] is a single bond. In some embodiments is a compound of Formula (I), (II), or (III), or a pharmaceutically acceptable salt or solvate thereof, wherein [symbol] is a double bond.

Any combination of the groups described above for the various variables is contemplated herein. Throughout the specification, groups and substituents thereof are chosen by one skilled in the field to provide stable moieties and compounds.

In some embodiments is a compound, or a pharmaceutically acceptable salt, solvate, or stereoisomer thereof, having a structure selected from:

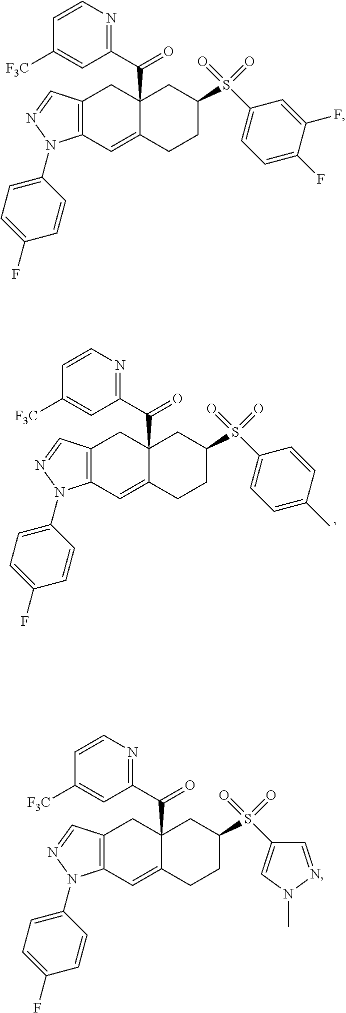 [SMILES: O=C(c1cc(C(F)(F)F)ccn1)[C@]12Cc3cnn(-c4ccc(F)cc4)c3C=C1CC[C@H](S(=O)(=O)c1ccc(F)c(F)c1)C2 ; Cc1ccc(S(=O)(=O)[C@H]2CCC3=Cc4c(cnn4-c4ccc(F)cc4)C[C@]3(C(=O)c3cc(C(F)(F)F)ccn3)C2)cc1 ; Cn1cc(S(=O)(=O)[C@H]2CCC3=Cc4c(cnn4-c4ccc(F)cc4)C[C@]3(C(=O)c3cc(C(F)(F)F)ccn3)C2)cn1]

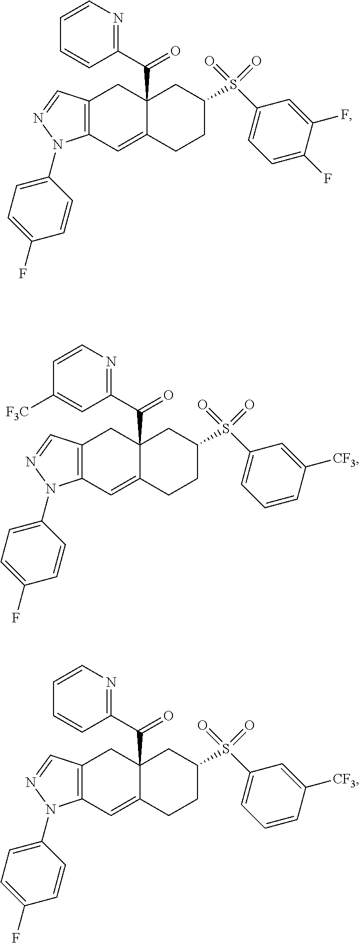 [SMILES: O=C(c1ccccn1)[C@]12Cc3cnn(-c4ccc(F)cc4)c3C=C1CC[C@@H](S(=O)(=O)c1ccc(F)c(F)c1)C2 ; O=C(c1cc(C(F)(F)F)ccn1)[C@]12Cc3cnn(-c4ccc(F)cc4)c3C=C1CC[C@@H](S(=O)(=O)c1cccc(C(F)(F)F)c1)C2 ; O=C(c1ccccn1)[C@]12Cc3cnn(-c4ccc(F)cc4)c3C=C1CC[C@@H](S(=O)(=O)c1cccc(C(F)(F)F)c1)C2]

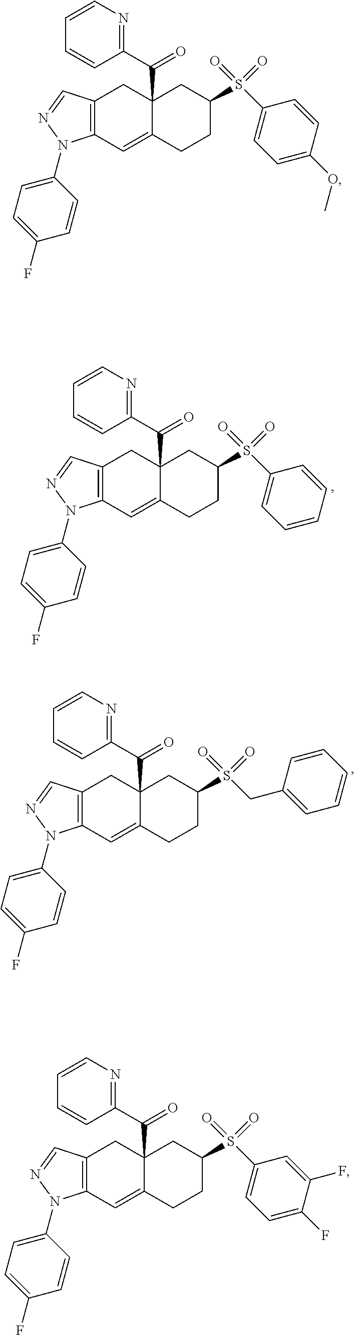 [SMILES: COc1ccc(S(=O)(=O)[C@H]2CCC3=Cc4c(cnn4-c4ccc(F)cc4)C[C@]3(C(=O)c3ccccn3)C2)cc1 ; O=C(c1ccccn1)[C@]12Cc3cnn(-c4ccc(F)cc4)c3C=C1CC[C@H](S(=O)(=O)c1ccccc1)C2 ; O=C(c1ccccn1)[C@]12Cc3cnn(-c4ccc(F)cc4)c3C=C1CC[C@H](S(=O)(=O)Cc1ccccc1)C2 ; O=C(c1ccccn1)[C@]12Cc3cnn(-c4ccc(F)cc4)c3C=C1CC[C@H](S(=O)(=O)c1ccc(F)c(F)c1)C2]

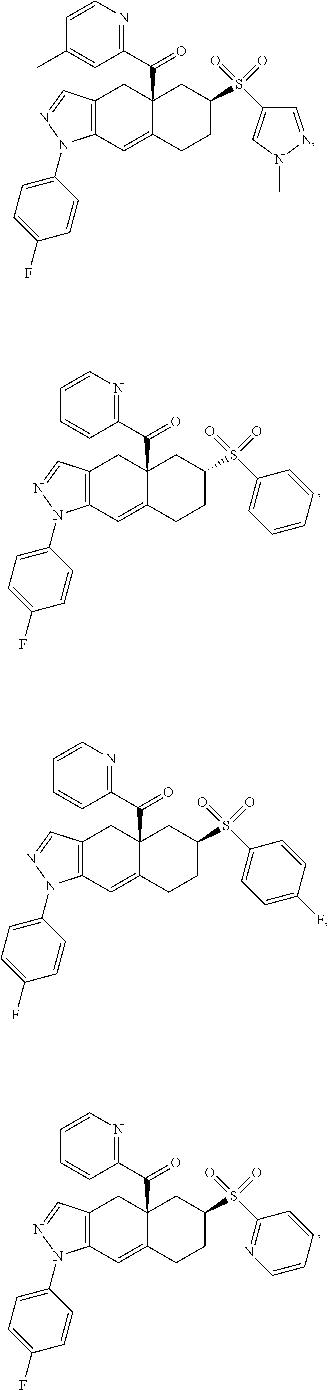 [SMILES: Cc1ccnc(C(=O)[C@]23Cc4cnn(-c5ccc(F)cc5)c4C=C2CC[C@H](S(=O)(=O)c2cnn(C)c2)C3)c1 ; O=C(c1ccccn1)[C@]12Cc3cnn(-c4ccc(F)cc4)c3C=C1CC[C@@H](S(=O)(=O)c1ccccc1)C2 ; O=C(c1ccccn1)[C@]12Cc3cnn(-c4ccc(F)cc4)c3C=C1CC[C@H](S(=O)(=O)c1ccc(F)cc1)C2 ; O=C(c1ccccn1)[C@]12Cc3cnn(-c4ccc(F)cc4)c3C=C1CC[C@H](S(=O)(=O)c1ccccn1)C2]

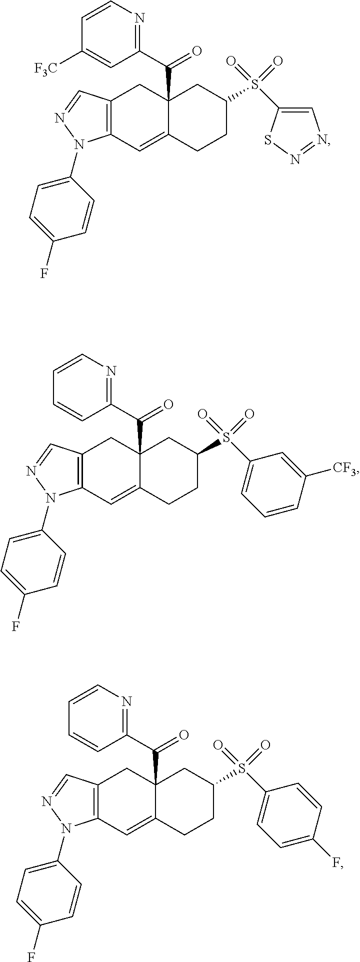

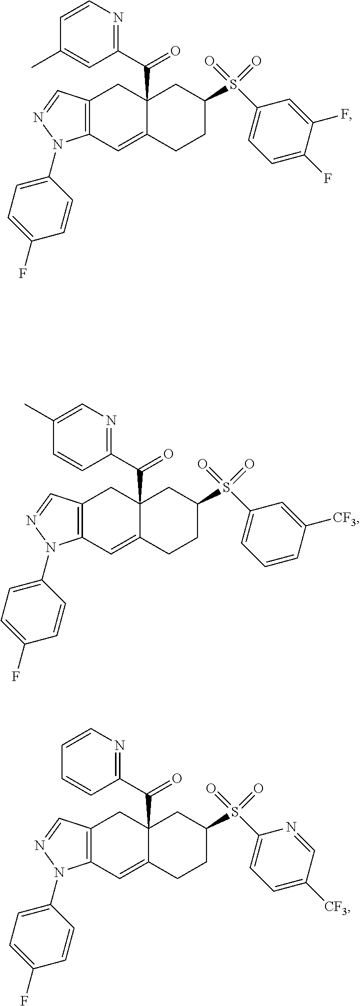 [SMILES: Cc1ccnc(C(=O)[C@]23Cc4cnn(-c5ccc(F)cc5)c4C=C2CC[C@H](S(=O)(=O)c2ccc(F)c(F)c2)C3)c1 ; Cc1ccc(C(=O)[C@]23Cc4cnn(-c5ccc(F)cc5)c4C=C2CC[C@H](S(=O)(=O)c2cccc(C(F)(F)F)c2)C3)nc1 ; O=C(c1ccccn1)[C@]12Cc3cnn(-c4ccc(F)cc4)c3C=C1CC[C@H](S(=O)(=O)c1ccc(C(F)(F)F)cn1)C2]

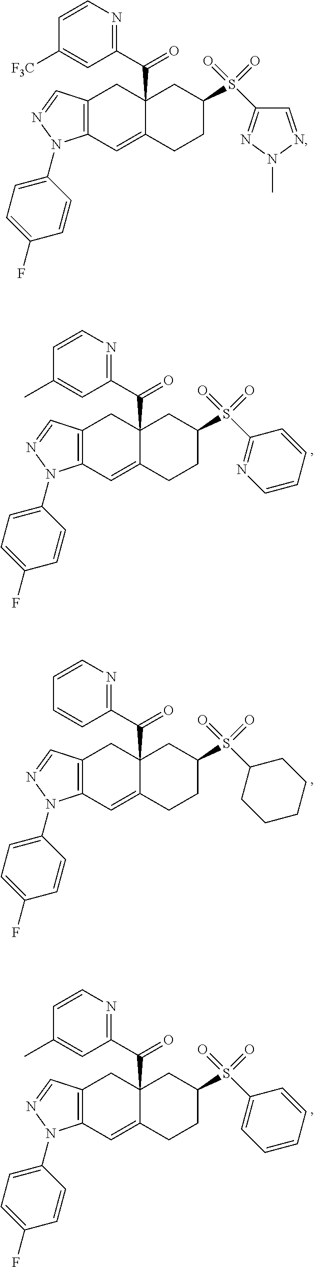 [SMILES: Cn1ncc(S(=O)(=O)[C@H]2CCC3=Cc4c(cnn4-c4ccc(F)cc4)C[C@]3(C(=O)c3cc(C(F)(F)F)ccn3)C2)n1 ; Cc1ccnc(C(=O)[C@]23Cc4cnn(-c5ccc(F)cc5)c4C=C2CC[C@H](S(=O)(=O)c2ccccn2)C3)c1 ; O=C(c1ccccn1)[C@]12Cc3cnn(-c4ccc(F)cc4)c3C=C1CC[C@H](S(=O)(=O)C1CCCCC1)C2 ; Cc1ccnc(C(=O)[C@]23Cc4cnn(-c5ccc(F)cc5)c4C=C2CC[C@H](S(=O)(=O)c2ccccc2)C3)c1]

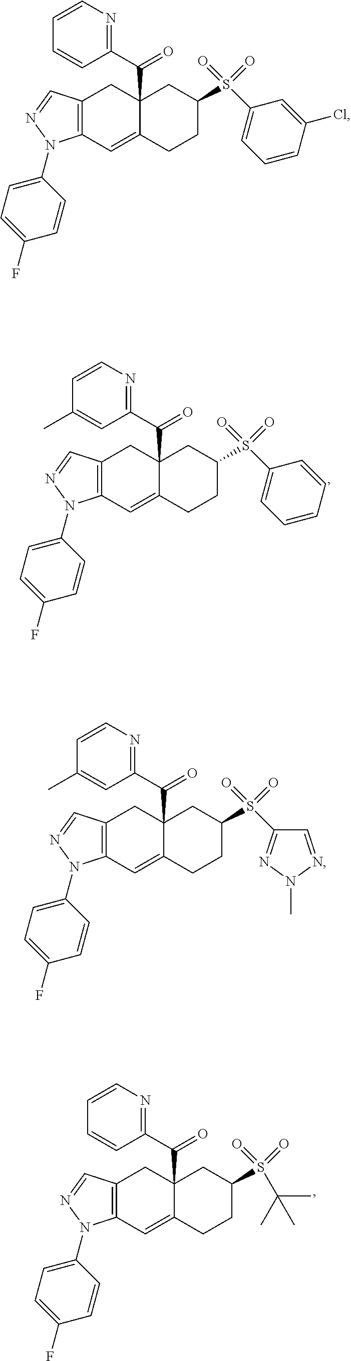 [SMILES: O=C(c1ccccn1)[C@]12Cc3cnn(-c4ccc(F)cc4)c3C=C1CC[C@H](S(=O)(=O)c1cccc(Cl)c1)C2 ; Cc1ccnc(C(=O)[C@]23Cc4cnn(-c5ccc(F)cc5)c4C=C2CC[C@@H](S(=O)(=O)c2ccccc2)C3)c1 ; Cc1ccnc(C(=O)[C@]23Cc4cnn(-c5ccc(F)cc5)c4C=C2CC[C@H](S(=O)(=O)c2cnn(C)n2)C3)c1 ; CC(C)(C)S(=O)(=O)[C@H]1CCC2=Cc3c(cnn3-c3ccc(F)cc3)C[C@]2(C(=O)c2ccccn2)C1]

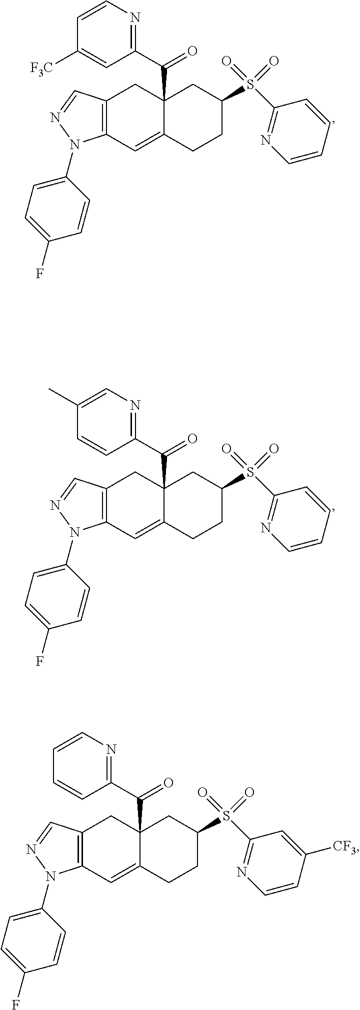 [SMILES: O=C(c1cc(C(F)(F)F)ccn1)[C@]12Cc3cnn(-c4ccc(F)cc4)c3C=C1CC[C@H](S(=O)(=O)c1ccccn1)C2 ; Cc1ccc(C(=O)[C@]23Cc4cnn(-c5ccc(F)cc5)c4C=C2CC[C@H](S(=O)(=O)c2ccccn2)C3)nc1 ; O=C(c1ccccn1)[C@]12Cc3cnn(-c4ccc(F)cc4)c3C=C1CC[C@H](S(=O)(=O)c1cc(C(F)(F)F)ccn1)C2]

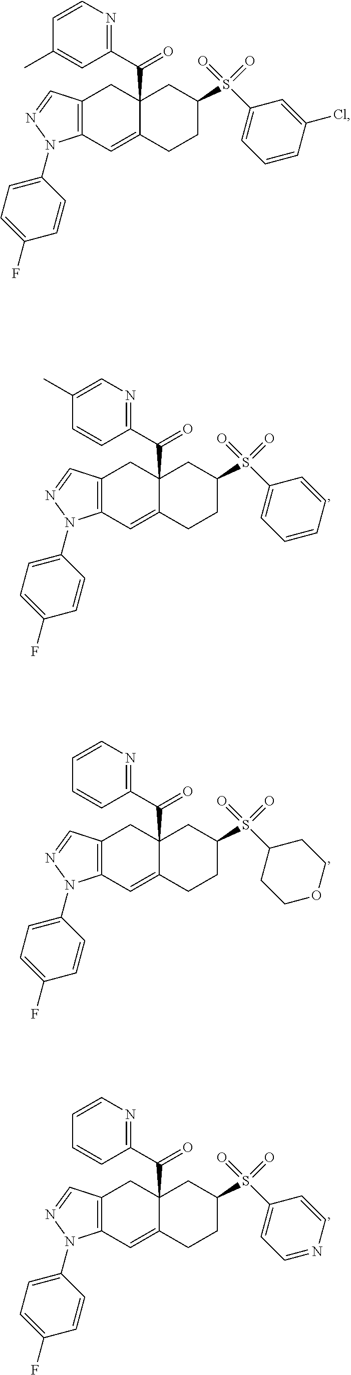 [SMILES: Cc1ccnc(C(=O)[C@]23Cc4cnn(-c5ccc(F)cc5)c4C=C2CC[C@H](S(=O)(=O)c2cccc(Cl)c2)C3)c1 ; Cc1ccc(C(=O)[C@]23Cc4cnn(-c5ccc(F)cc5)c4C=C2CC[C@H](S(=O)(=O)c2ccccc2)C3)nc1 ; O=C(c1ccccn1)[C@]12Cc3cnn(-c4ccc(F)cc4)c3C=C1CC[C@H](S(=O)(=O)C1CCOCC1)C2 ; O=C(c1ccccn1)[C@]12Cc3cnn(-c4ccc(F)cc4)c3C=C1CC[C@H](S(=O)(=O)c1ccncc1)C2]

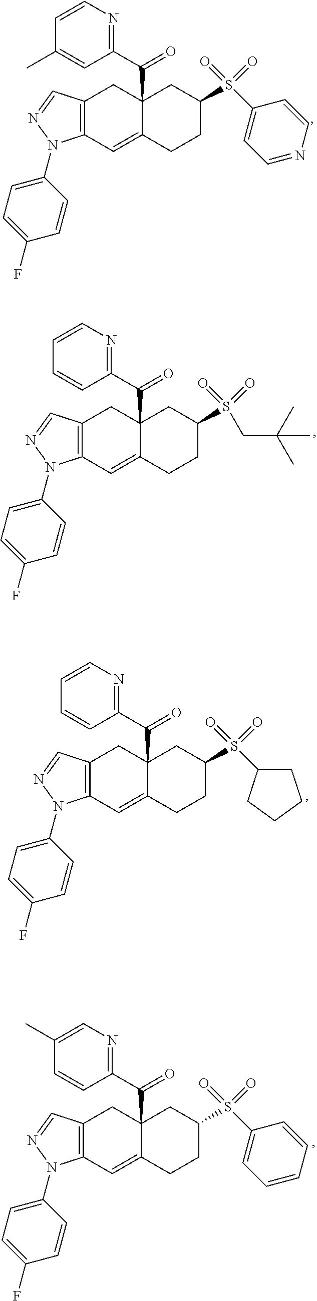 [SMILES: Cc1ccnc(C(=O)[C@]23Cc4cnn(-c5ccc(F)cc5)c4C=C2CC[C@H](S(=O)(=O)c2ccncc2)C3)c1 ; CC(C)(C)CS(=O)(=O)[C@H]1CCC2=Cc3c(cnn3-c3ccc(F)cc3)C[C@]2(C(=O)c2ccccn2)C1 ; O=C(c1ccccn1)[C@]12Cc3cnn(-c4ccc(F)cc4)c3C=C1CC[C@H](S(=O)(=O)C1CCCC1)C2 ; Cc1ccc(C(=O)[C@]23Cc4cnn(-c5ccc(F)cc5)c4C=C2CC[C@@H](S(=O)(=O)c2ccccc2)C3)nc1]

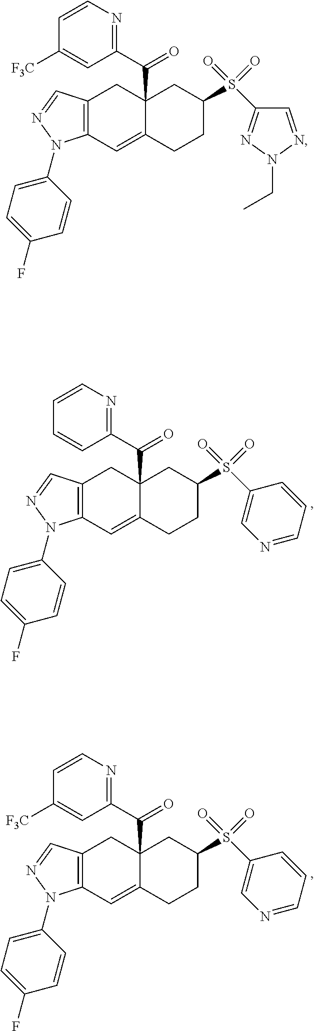 [SMILES: CCn1ncc(S(=O)(=O)[C@H]2CCC3=Cc4c(cnn4-c4ccc(F)cc4)C[C@]3(C(=O)c3cc(C(F)(F)F)ccn3)C2)n1 ; O=C(c1ccccn1)[C@]12Cc3cnn(-c4ccc(F)cc4)c3C=C1CC[C@H](S(=O)(=O)c1cccnc1)C2 ; O=C(c1cc(C(F)(F)F)ccn1)[C@]12Cc3cnn(-c4ccc(F)cc4)c3C=C1CC[C@H](S(=O)(=O)c1cccnc1)C2]

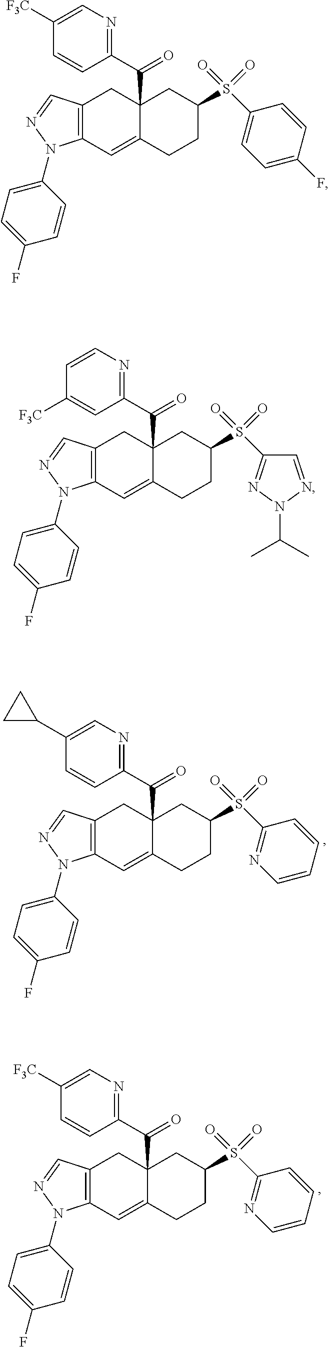 [SMILES: O=C(c1ccc(C(F)(F)F)cn1)[C@]12Cc3cnn(-c4ccc(F)cc4)c3C=C1CC[C@H](S(=O)(=O)c1ccc(F)cc1)C2 ; CC(C)n1ncc(S(=O)(=O)[C@H]2CCC3=Cc4c(cnn4-c4ccc(F)cc4)C[C@]3(C(=O)c3cc(C(F)(F)F)ccn3)C2)n1 ; O=C(c1ccc(C2CC2)cn1)[C@]12Cc3cnn(-c4ccc(F)cc4)c3C=C1CC[C@H](S(=O)(=O)c1ccccn1)C2 ; O=C(c1ccc(C(F)(F)F)cn1)[C@]12Cc3cnn(-c4ccc(F)cc4)c3C=C1CC[C@H](S(=O)(=O)c1ccccn1)C2]

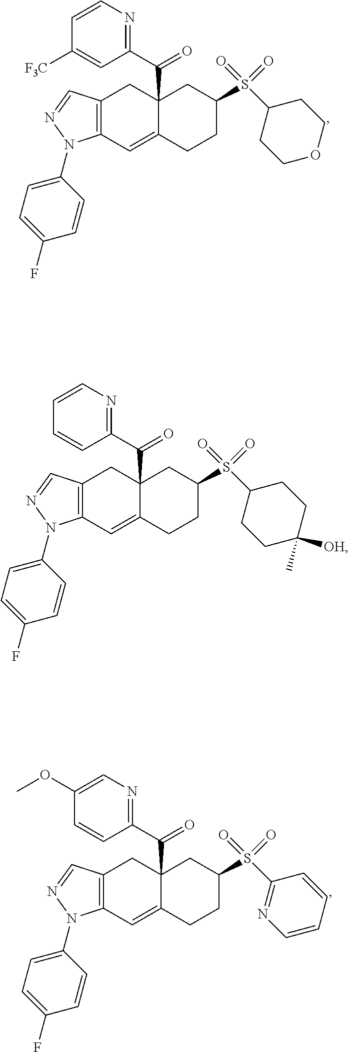

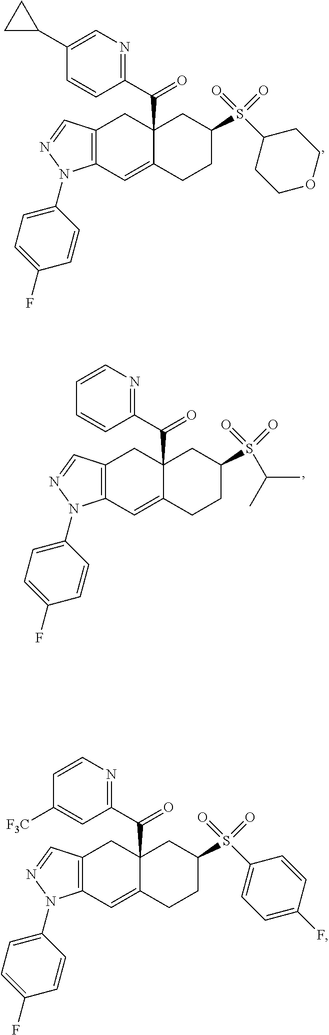 [SMILES: O=C(c1ccc(C2CC2)cn1)[C@]12Cc3cnn(-c4ccc(F)cc4)c3C=C1CC[C@H](S(=O)(=O)C1CCOCC1)C2 ; CC(C)S(=O)(=O)[C@H]1CCC2=Cc3c(cnn3-c3ccc(F)cc3)C[C@]2(C(=O)c2ccccn2)C1 ; O=C(c1cc(C(F)(F)F)ccn1)[C@]12Cc3cnn(-c4ccc(F)cc4)c3C=C1CC[C@H](S(=O)(=O)c1ccc(F)cc1)C2]

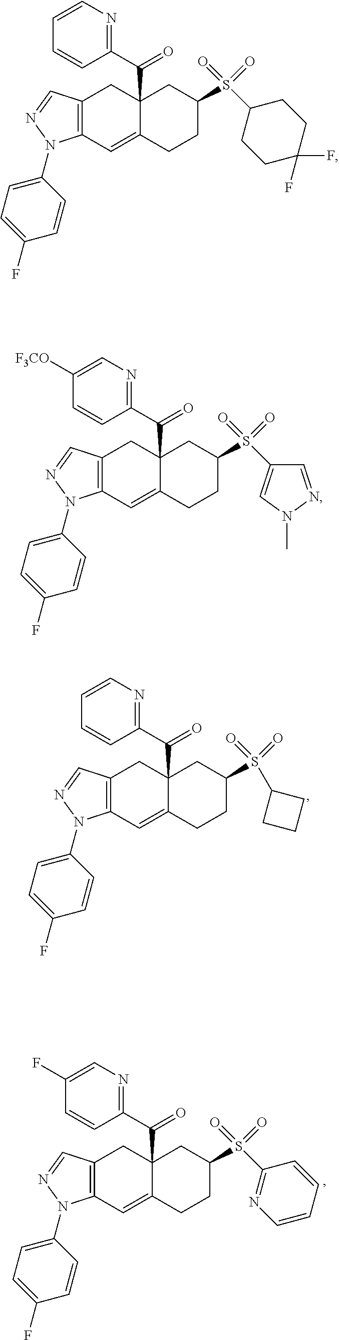 [SMILES: O=C(c1ccccn1)[C@]12Cc3cnn(-c4ccc(F)cc4)c3C=C1CC[C@H](S(=O)(=O)C1CCC(F)(F)CC1)C2 ; Cn1cc(S(=O)(=O)[C@H]2CCC3=Cc4c(cnn4-c4ccc(F)cc4)C[C@]3(C(=O)c3ccc(OC(F)(F)F)cn3)C2)cn1 ; O=C(c1ccccn1)[C@]12Cc3cnn(-c4ccc(F)cc4)c3C=C1CC[C@H](S(=O)(=O)C1CCC1)C2 ; O=C(c1ccc(F)cn1)[C@]12Cc3cnn(-c4ccc(F)cc4)c3C=C1CC[C@H](S(=O)(=O)c1ccccn1)C2]

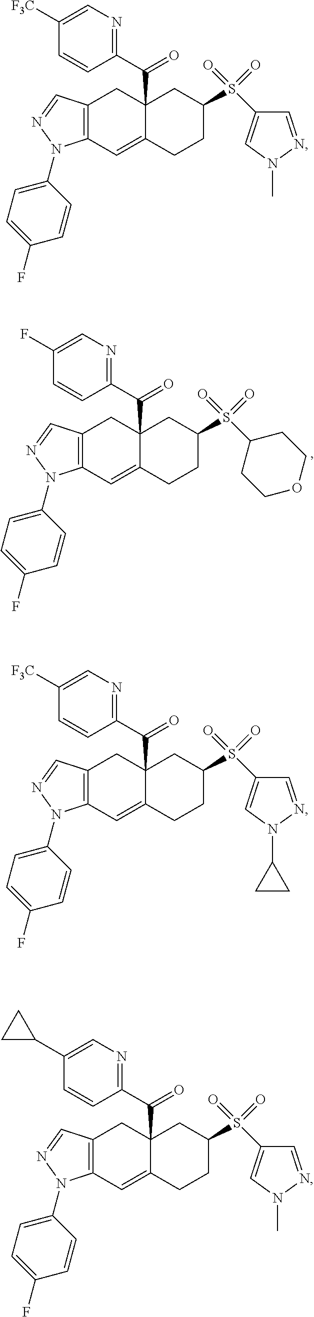 [SMILES: Cn1cc(S(=O)(=O)[C@H]2CCC3=Cc4c(cnn4-c4ccc(F)cc4)C[C@]3(C(=O)c3ccc(C(F)(F)F)cn3)C2)cn1 ; O=C(c1ccc(F)cn1)[C@]12Cc3cnn(-c4ccc(F)cc4)c3C=C1CC[C@H](S(=O)(=O)C1CCOCC1)C2 ; O=C(c1ccc(C(F)(F)F)cn1)[C@]12Cc3cnn(-c4ccc(F)cc4)c3C=C1CC[C@H](S(=O)(=O)c1cnn(C3CC3)c1)C2 ; Cn1cc(S(=O)(=O)[C@H]2CCC3=Cc4c(cnn4-c4ccc(F)cc4)C[C@]3(C(=O)c3ccc(C4CC4)cn3)C2)cn1]

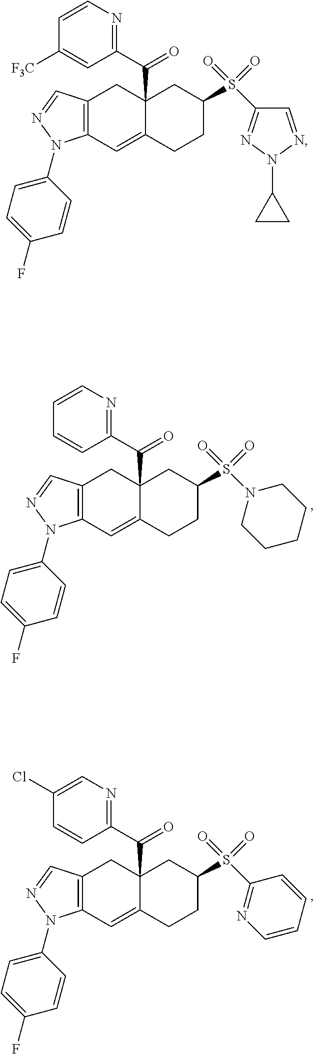 [SMILES: O=C(c1cc(C(F)(F)F)ccn1)[C@]12Cc3cnn(-c4ccc(F)cc4)c3C=C1CC[C@H](S(=O)(=O)c1cnn(C3CC3)n1)C2 ; O=C(c1ccccn1)[C@]12Cc3cnn(-c4ccc(F)cc4)c3C=C1CC[C@H](S(=O)(=O)N1CCCCC1)C2 ; O=C(c1ccc(Cl)cn1)[C@]12Cc3cnn(-c4ccc(F)cc4)c3C=C1CC[C@H](S(=O)(=O)c1ccccn1)C2]

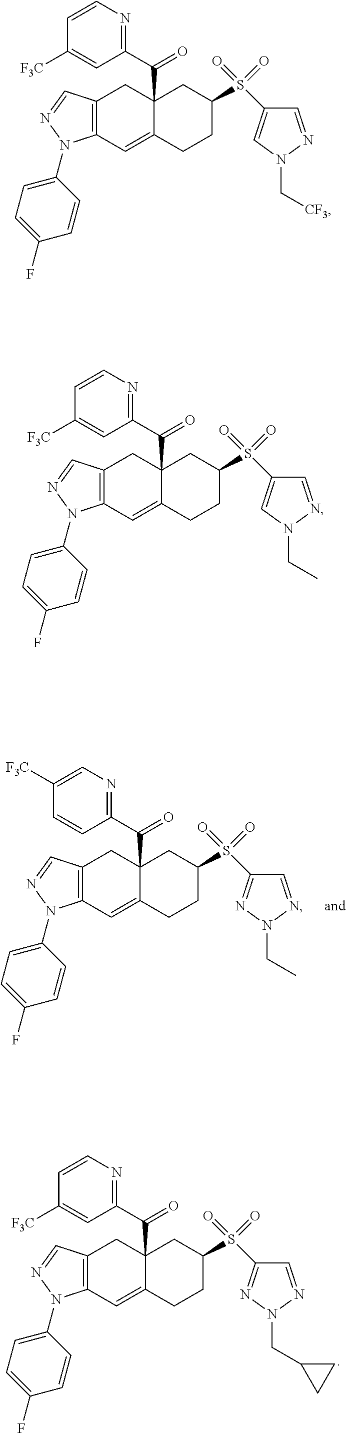 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)c1)C2 ; CCn1cc(S(=O)(=O)[C@H]2CCC3=Cc4c(cnn4-c4ccc(F)cc4)C[C@]3(C(=O)c3cc(C(F)(F)F)ccn3)C2)cn1 ; CCn1ncc(S(=O)(=O)[C@H]2CCC3=Cc4c(cnn4-c4ccc(F)cc4)C[C@]3(C(=O)c3ccc(C(F)(F)F)cn3)C2)n1 ; O=C(c1cc(C(F)(F)F)ccn1)[C@]12Cc3cnn(-c4ccc(F)cc4)c3C=C1CC[C@H](S(=O)(=O)c1cnn(CC3CC3)n1)C2]

In some embodiments is a compound, or a pharmaceutically acceptable salt, solvate, or stereoisomer thereof, having a structure selected from:

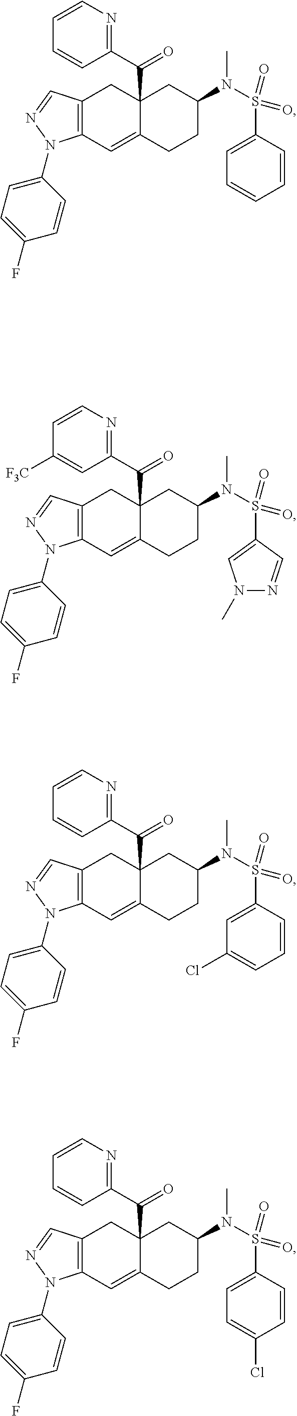 [SMILES: CN([C@H]1CCC2=Cc3c(cnn3-c3ccc(F)cc3)C[C@]2(C(=O)c2ccccn2)C1)S(=O)(=O)c1ccccc1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; CN([C@H]1CCC2=Cc3c(cnn3-c3ccc(F)cc3)C[C@]2(C(=O)c2ccccn2)C1)S(=O)(=O)c1cccc(Cl)c1 ; CN([C@H]1CCC2=Cc3c(cnn3-c3ccc(F)cc3)C[C@]2(C(=O)c2ccccn2)C1)S(=O)(=O)c1ccc(Cl)cc1]

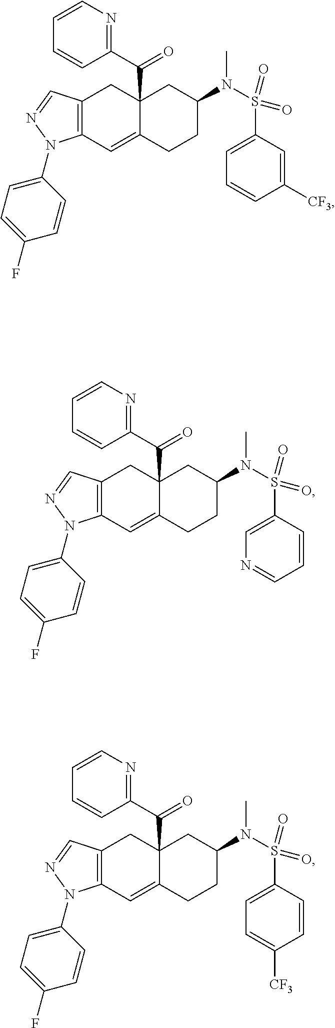 [SMILES: CN([C@H]1CCC2=Cc3c(cnn3-c3ccc(F)cc3)C[C@]2(C(=O)c2ccccn2)C1)S(=O)(=O)c1cccc(C(F)(F)F)c1 ; CN([C@H]1CCC2=Cc3c(cnn3-c3ccc(F)cc3)C[C@]2(C(=O)c2ccccn2)C1)S(=O)(=O)c1cccnc1 ; CN([C@H]1CCC2=Cc3c(cnn3-c3ccc(F)cc3)C[C@]2(C(=O)c2ccccn2)C1)S(=O)(=O)c1ccc(C(F)(F)F)cc1]

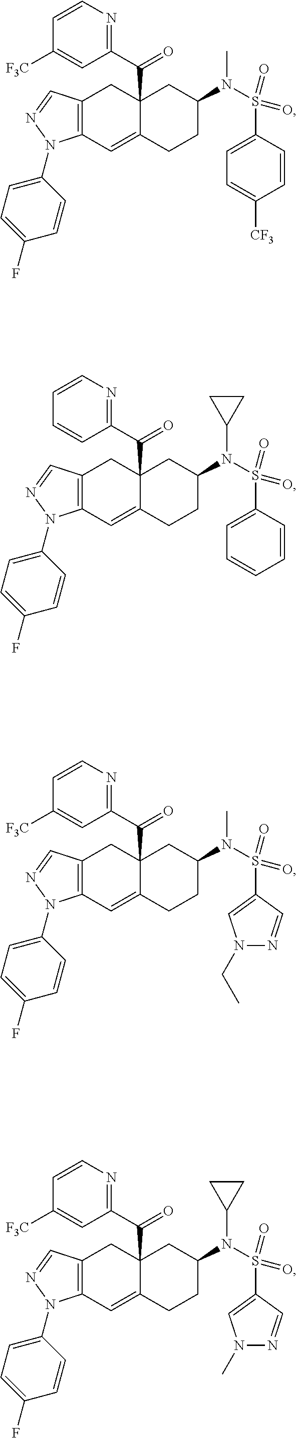 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccc(C(F)(F)F)cc1 ; O=C(c1ccccn1)[C@]12Cc3cnn(-c4ccc(F)cc4)c3C=C1CC[C@H](N(C1CC1)S(=O)(=O)c1ccccc1)C2 ; CCn1cc(S(=O)(=O)N(C)[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1]

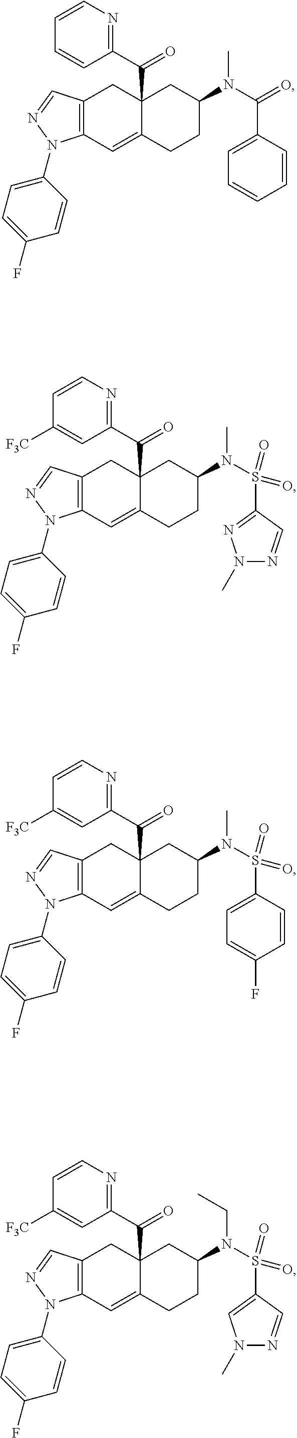 [SMILES: CN(C(=O)c1ccccc1)[C@H]1CCC2=Cc3c(cnn3-c3ccc(F)cc3)C[C@]2(C(=O)c2ccccn2)C1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1ccc(F)cc1 ; CCN([C@H]1CCC2=Cc3c(cnn3-c3ccc(F)cc3)C[C@]2(C(=O)c2cc(C(F)(F)F)ccn2)C1)S(=O)(=O)c1cnn(C)c1]

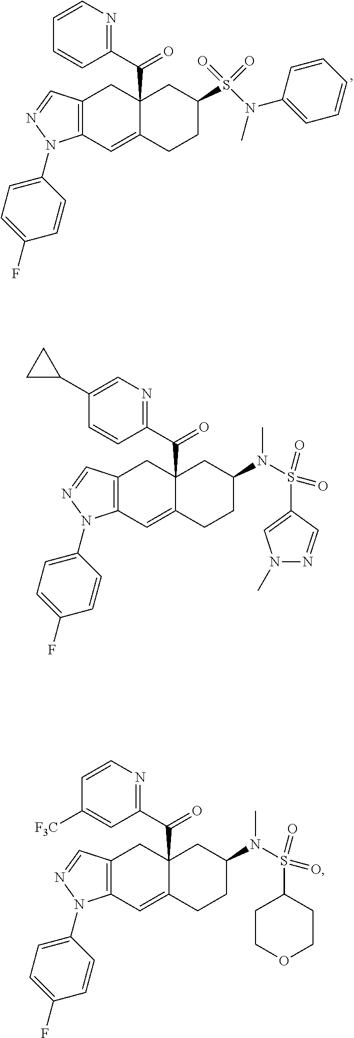 [SMILES: CN(c1ccccc1)S(=O)(=O)[C@H]1CCC2=Cc3c(cnn3-c3ccc(F)cc3)C[C@]2(C(=O)c2ccccn2)C1 ; CN([C@H]1CCC2=Cc3c(cnn3-c3ccc(F)cc3)C[C@]2(C(=O)c2ccc(C3CC3)cn2)C1)S(=O)(=O)c1cnn(C)c1 ; CN([C@H]1CCC2=Cc3c(cnn3-c3ccc(F)cc3)C[C@]2(C(=O)c2cc(C(F)(F)F)ccn2)C1)S(=O)(=O)C1CCOCC1]

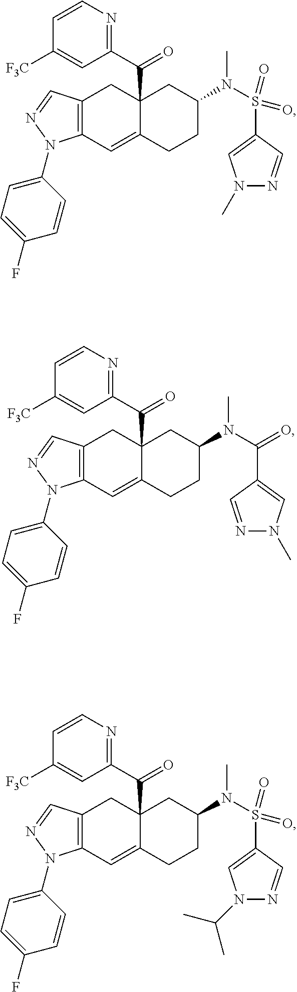 [SMILES: CN([C@@H]1CCC2=Cc3c(cnn3-c3ccc(F)cc3)C[C@]2(C(=O)c2cc(C(F)(F)F)ccn2)C1)S(=O)(=O)c1cnn(C)c1 ; CN(C(=O)c1cnn(C)c1)[C@H]1CCC2=Cc3c(cnn3-c3ccc(F)cc3)C[C@]2(C(=O)c2cc(C(F)(F)F)ccn2)C1 ; CC(C)n1cc(S(=O)(=O)N(C)[C@H]2CCC3=Cc4c(cnn4-c4ccc(F)cc4)C[C@]3(C(=O)c3cc(C(F)(F)F)ccn3)C2)cn1]

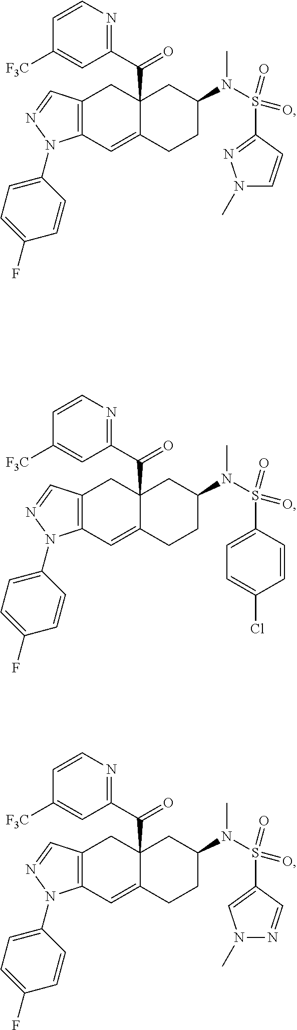 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1ccc(Cl)cc1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)c1]

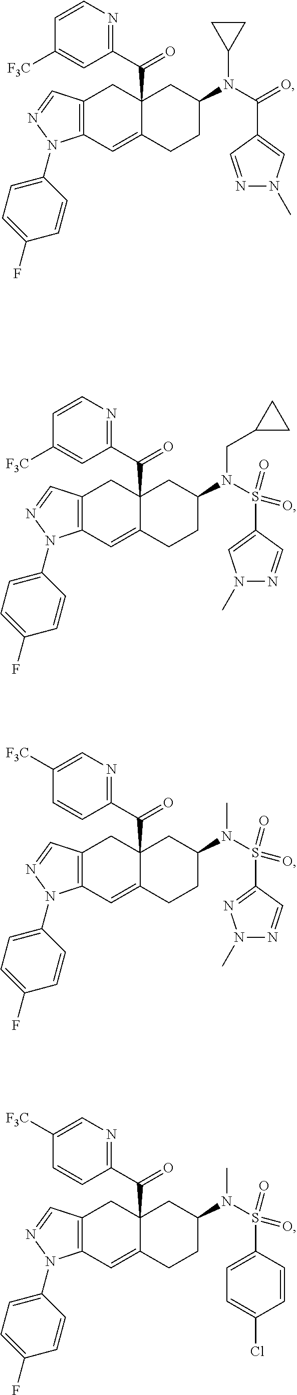 [SMILES: Cn1cc(C(=O)N(C2CC2)[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)cn1 ; CN([C@H]1CCC2=Cc3c(cnn3-c3ccc(F)cc3)C[C@]2(C(=O)c2ccc(C(F)(F)F)cn2)C1)S(=O)(=O)c1cnn(C)n1 ; CN([C@H]1CCC2=Cc3c(cnn3-c3ccc(F)cc3)C[C@]2(C(=O)c2ccc(C(F)(F)F)cn2)C1)S(=O)(=O)c1ccc(Cl)cc1]

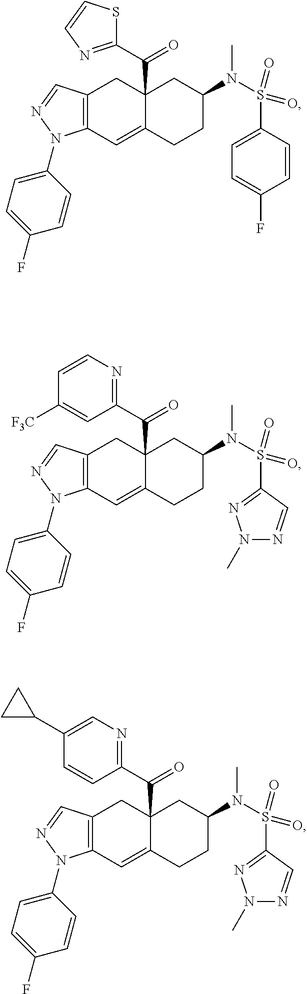 [SMILES: CN([C@H]1CCC2=Cc3c(cnn3-c3ccc(F)cc3)C[C@]2(C(=O)c2nccs2)C1)S(=O)(=O)c1ccc(F)cc1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)n1 ; CN([C@H]1CCC2=Cc3c(cnn3-c3ccc(F)cc3)C[C@]2(C(=O)c2ccc(C3CC3)cn2)C1)S(=O)(=O)c1cnn(C)n1]

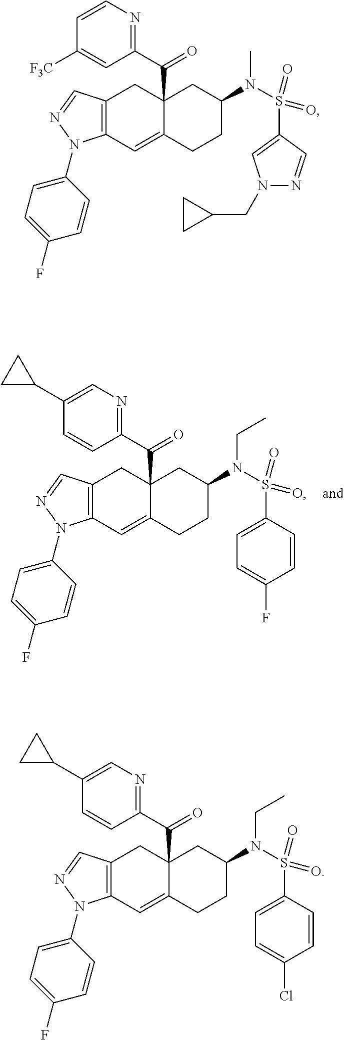 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(CC2CC2)c1 ; CCN([C@H]1CCC2=Cc3c(cnn3-c3ccc(F)cc3)C[C@]2(C(=O)c2ccc(C3CC3)cn2)C1)S(=O)(=O)c1ccc(F)cc1 ; CCN([C@H]1CCC2=Cc3c(cnn3-c3ccc(F)cc3)C[C@]2(C(=O)c2ccc(C3CC3)cn2)C1)S(=O)(=O)c1ccc(Cl)cc1]

In some embodiments is a compound, or a pharmaceutically acceptable salt, solvate, or stereoisomer thereof, having a structure selected from:

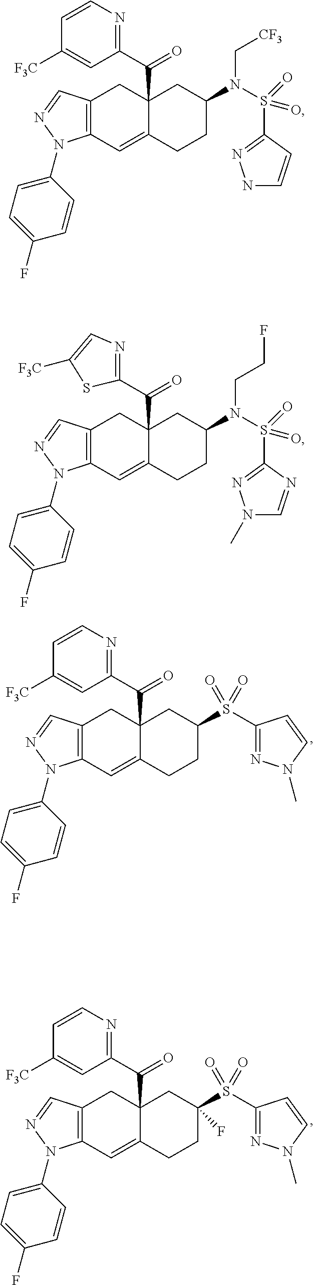 [SMILES: O=C(c1cc(C(F)(F)F)ccn1)[C@]12Cc3cnn(-c4ccc(F)cc4)c3C=C1CC[C@H](N(CC(F)(F)F)S(=O)(=O)c1cc[nH]n1)C2 ; Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)[C@@]2(F)CCC3=Cc4c(cnn4-c4ccc(F)cc4)C[C@]3(C(=O)c3cc(C(F)(F)F)ccn3)C2)n1]

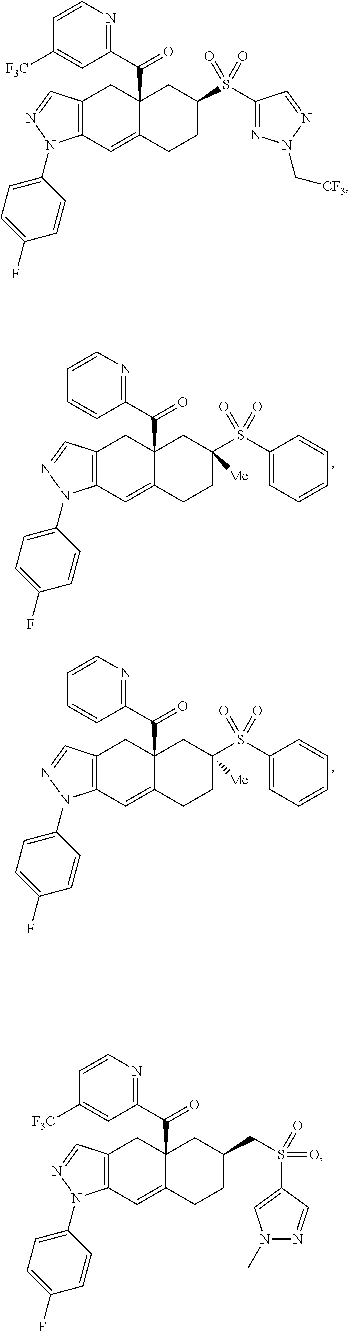 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)n1)C2 ; C[C@@]1(S(=O)(=O)c2ccccc2)CCC2=Cc3c(cnn3-c3ccc(F)cc3)C[C@]2(C(=O)c2ccccn2)C1 ; C[C@]1(S(=O)(=O)c2ccccc2)CCC2=Cc3c(cnn3-c3ccc(F)cc3)C[C@]2(C(=O)c2ccccn2)C1 ; Cn1cc(S(=O)(=O)C[C@H]2CCC3=Cc4c(cnn4-c4ccc(F)cc4)C[C@]3(C(=O)c3cc(C(F)(F)F)ccn3)C2)cn1]

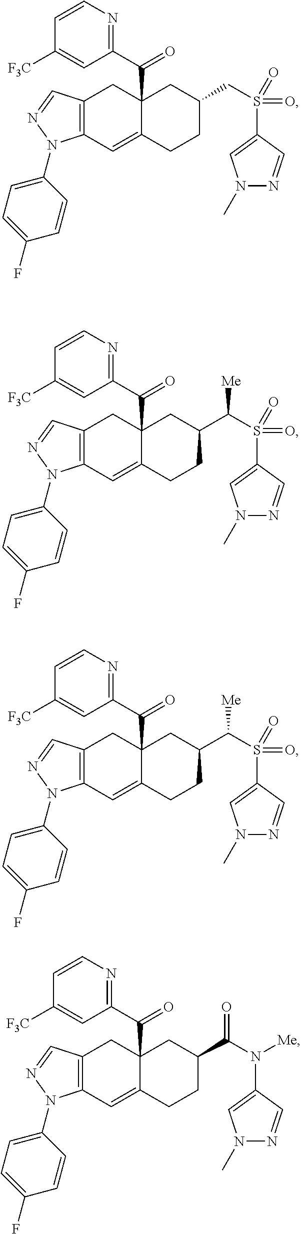 [SMILES: Cn1cc(S(=O)(=O)C[C@@H]2CCC3=Cc4c(cnn4-c4ccc(F)cc4)C[C@]3(C(=O)c3cc(C(F)(F)F)ccn3)C2)cn1 ; C[C@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; C[C@@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; CN(C(=O)[C@H]1CCC2=Cc3c(cnn3-c3ccc(F)cc3)C[C@]2(C(=O)c2cc(C(F)(F)F)ccn2)C1)c1cnn(C)c1]

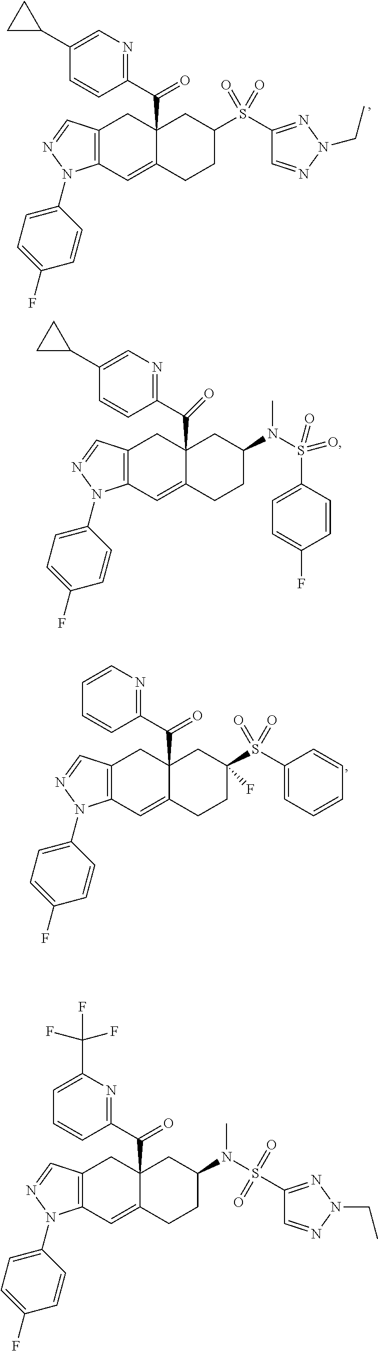

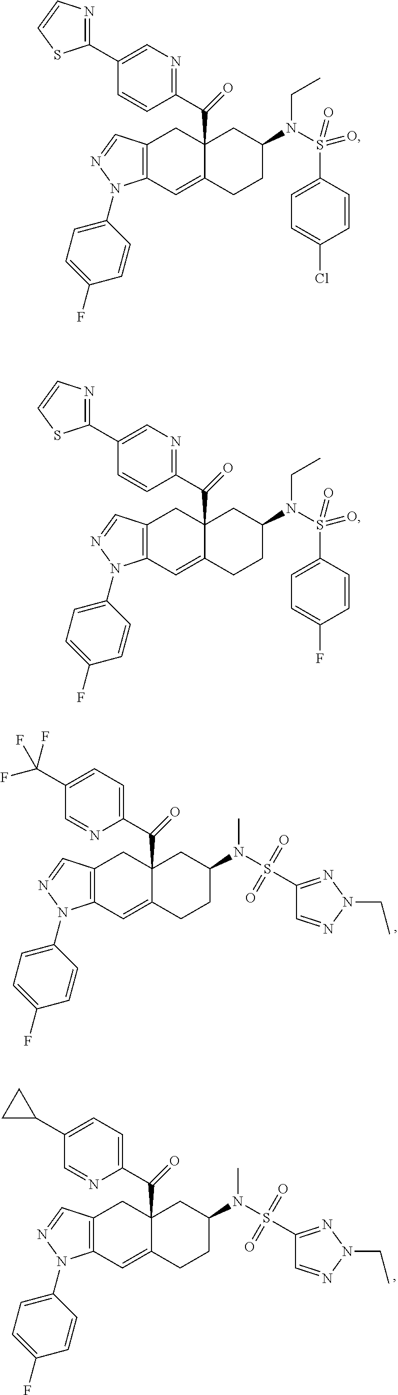 [SMILES: CCN([C@H]1CCC2=Cc3c(cnn3-c3ccc(F)cc3)C[C@]2(C(=O)c2ccc(-c3nccs3)cn2)C1)S(=O)(=O)c1ccc(Cl)cc1 ; CCN([C@H]1CCC2=Cc3c(cnn3-c3ccc(F)cc3)C[C@]2(C(=O)c2ccc(-c3nccs3)cn2)C1)S(=O)(=O)c1ccc(F)cc1 ; CCn1ncc(S(=O)(=O)N(C)[C@H]2CCC3=Cc4c(cnn4-c4ccc(F)cc4)C[C@]3(C(=O)c3ccc(C(F)(F)F)cn3)C2)n1 ; CCn1ncc(S(=O)(=O)N(C)[C@H]2CCC3=Cc4c(cnn4-c4ccc(F)cc4)C[C@]3(C(=O)c3ccc(C4CC4)cn3)C2)n1]

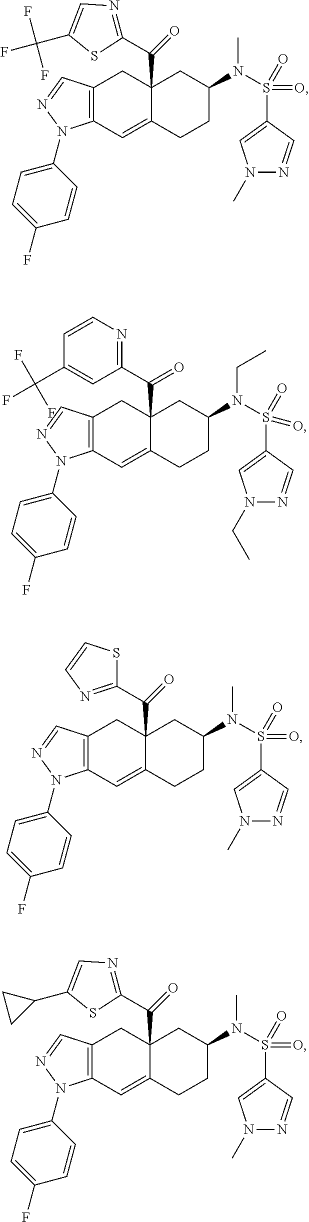 [SMILES: CN([C@H]1CCC2=Cc3c(cnn3-c3ccc(F)cc3)C[C@]2(C(=O)c2ncc(C(F)(F)F)s2)C1)S(=O)(=O)c1cnn(C)c1 ; CCN([C@H]1CCC2=Cc3c(cnn3-c3ccc(F)cc3)C[C@]2(C(=O)c2cc(C(F)(F)F)ccn2)C1)S(=O)(=O)c1cnn(CC)c1 ; CN([C@H]1CCC2=Cc3c(cnn3-c3ccc(F)cc3)C[C@]2(C(=O)c2nccs2)C1)S(=O)(=O)c1cnn(C)c1 ; CN([C@H]1CCC2=Cc3c(cnn3-c3ccc(F)cc3)C[C@]2(C(=O)c2ncc(C3CC3)s2)C1)S(=O)(=O)c1cnn(C)c1]

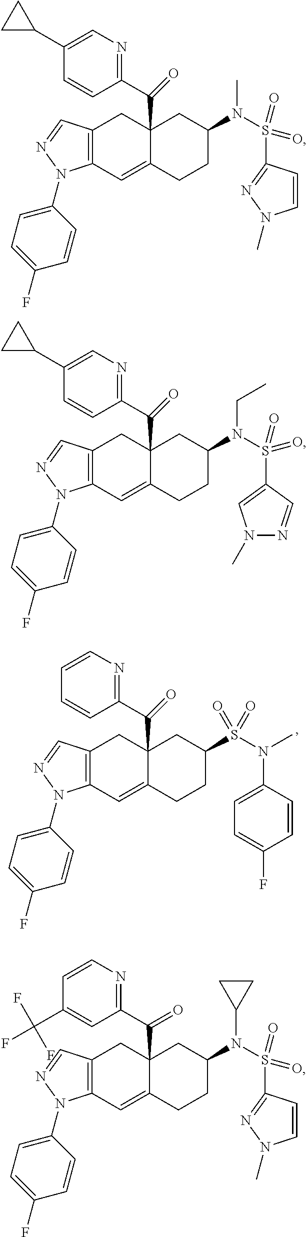

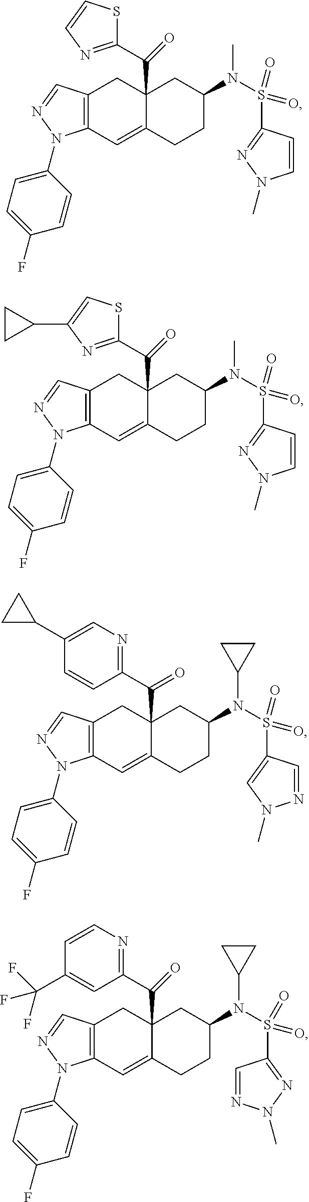

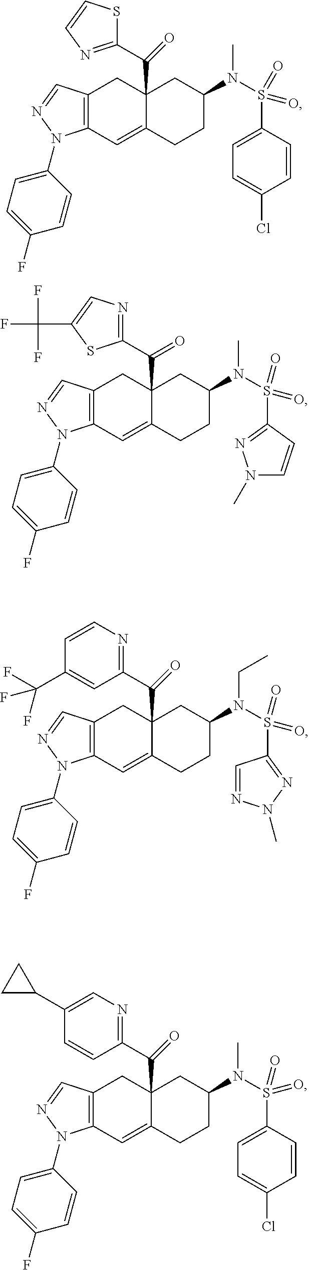

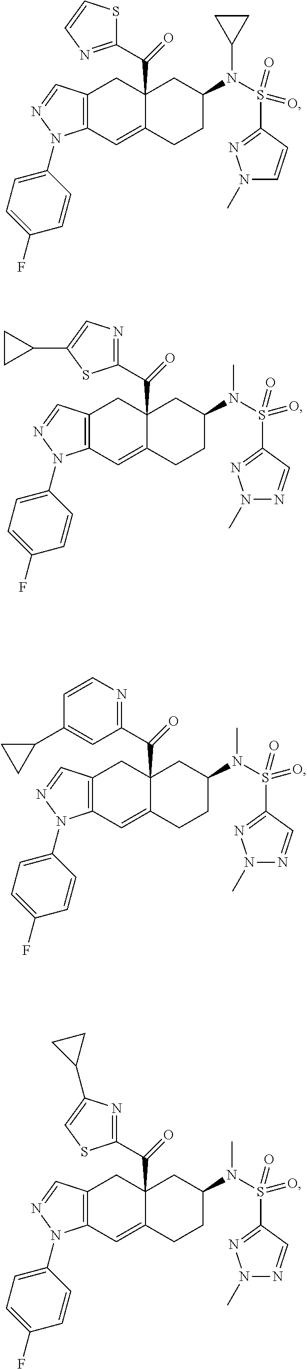 [SMILES: Cn1ccc(S(=O)(=O)N(C2CC2)[C@H]2CCC3=Cc4c(cnn4-c4ccc(F)cc4)C[C@]3(C(=O)c3nccs3)C2)n1 ; CN([C@H]1CCC2=Cc3c(cnn3-c3ccc(F)cc3)C[C@]2(C(=O)c2ncc(C3CC3)s2)C1)S(=O)(=O)c1cnn(C)n1 ; CN([C@H]1CCC2=Cc3c(cnn3-c3ccc(F)cc3)C[C@]2(C(=O)c2cc(C3CC3)ccn2)C1)S(=O)(=O)c1cnn(C)n1 ; CN([C@H]1CCC2=Cc3c(cnn3-c3ccc(F)cc3)C[C@]2(C(=O)c2nc(C3CC3)cs2)C1)S(=O)(=O)c1cnn(C)n1]

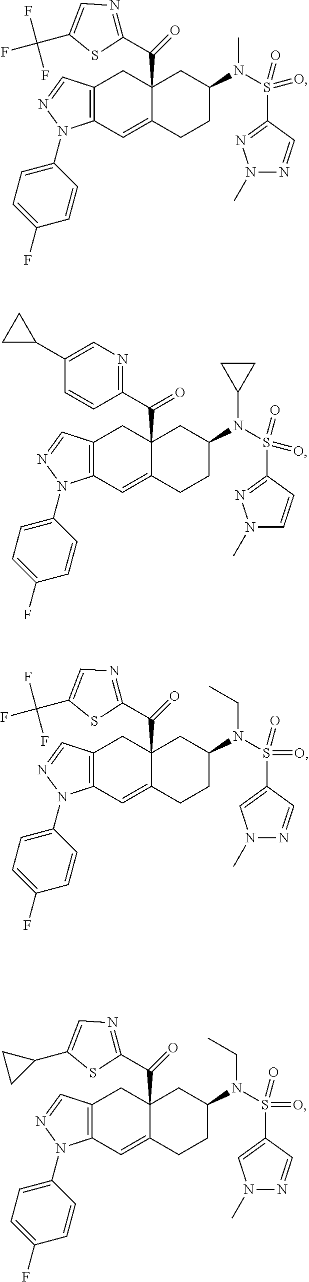 [SMILES: CN([C@H]1CCC2=Cc3c(cnn3-c3ccc(F)cc3)C[C@]2(C(=O)c2ncc(C(F)(F)F)s2)C1)S(=O)(=O)c1cnn(C)n1 ; Cn1ccc(S(=O)(=O)N(C2CC2)[C@H]2CCC3=Cc4c(cnn4-c4ccc(F)cc4)C[C@]3(C(=O)c3ccc(C4CC4)cn3)C2)n1 ; CCN([C@H]1CCC2=Cc3c(cnn3-c3ccc(F)cc3)C[C@]2(C(=O)c2ncc(C(F)(F)F)s2)C1)S(=O)(=O)c1cnn(C)c1 ; CCN([C@H]1CCC2=Cc3c(cnn3-c3ccc(F)cc3)C[C@]2(C(=O)c2ncc(C3CC3)s2)C1)S(=O)(=O)c1cnn(C)c1]

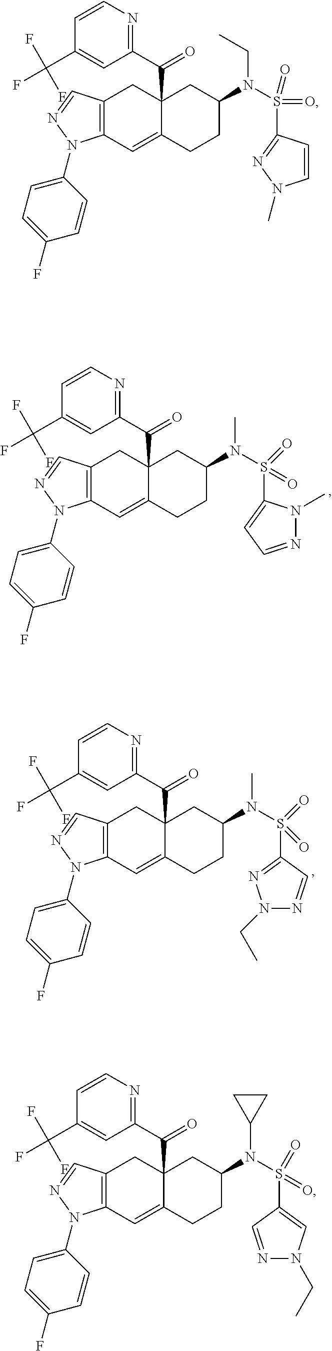 [SMILES: CCN([C@H]1CCC2=Cc3c(cnn3-c3ccc(F)cc3)C[C@]2(C(=O)c2cc(C(F)(F)F)ccn2)C1)S(=O)(=O)c1ccn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1ccnn1C ; CCn1ncc(S(=O)(=O)N(C)[C@H]2CCC3=Cc4c(cnn4-c4ccc(F)cc4)C[C@]3(C(=O)c3cc(C(F)(F)F)ccn3)C2)n1 ; CCn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1]

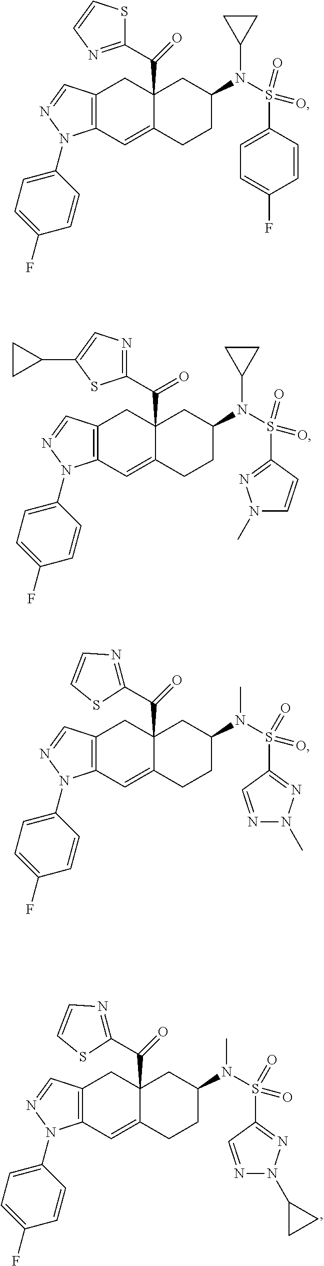 [SMILES: O=C(c1nccs1)[C@]12Cc3cnn(-c4ccc(F)cc4)c3C=C1CC[C@H](N(C1CC1)S(=O)(=O)c1ccc(F)cc1)C2 ; Cn1ccc(S(=O)(=O)N(C2CC2)[C@H]2CCC3=Cc4c(cnn4-c4ccc(F)cc4)C[C@]3(C(=O)c3ncc(C4CC4)s3)C2)n1 ; CN([C@H]1CCC2=Cc3c(cnn3-c3ccc(F)cc3)C[C@]2(C(=O)c2nccs2)C1)S(=O)(=O)c1cnn(C)n1 ; CN([C@H]1CCC2=Cc3c(cnn3-c3ccc(F)cc3)C[C@]2(C(=O)c2nccs2)C1)S(=O)(=O)c1cnn(C2CC2)n1]

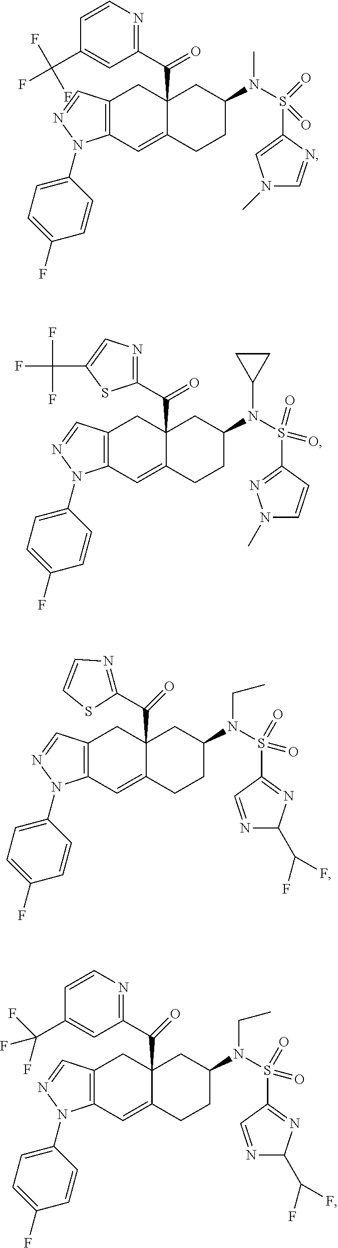 [SMILES: CN([C@H]1CCC2=Cc3c(cnn3-c3ccc(F)cc3)C[C@]2(C(=O)c2cc(C(F)(F)F)ccn2)C1)S(=O)(=O)c1cn(C)cn1 ; Cn1ccc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; CCN([C@H]1CCC2=Cc3c(cnn3-c3ccc(F)cc3)C[C@]2(C(=O)c2nccs2)C1)S(=O)(=O)C1=NC(C(F)F)N=C1 ; CCN([C@H]1CCC2=Cc3c(cnn3-c3ccc(F)cc3)C[C@]2(C(=O)c2cc(C(F)(F)F)ccn2)C1)S(=O)(=O)C1=NC(C(F)F)N=C1]

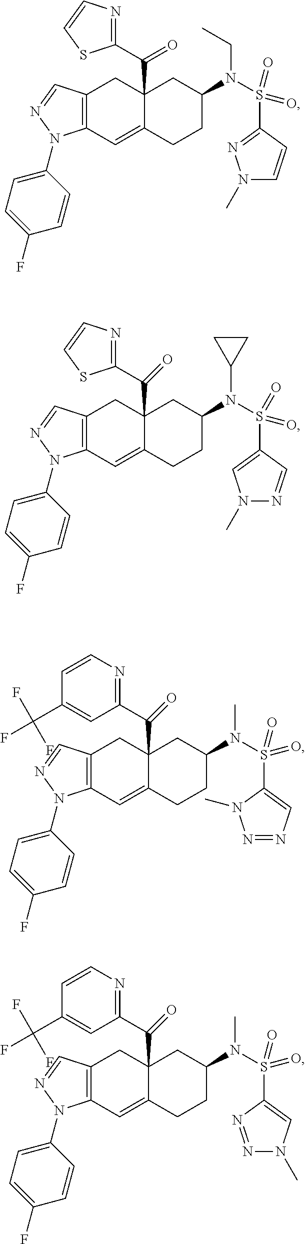 [SMILES: CCN([C@H]1CCC2=Cc3c(cnn3-c3ccc(F)cc3)C[C@]2(C(=O)c2nccs2)C1)S(=O)(=O)c1ccn(C)n1 ; Cn1cc(S(=O)(=O)N(C2CC2)[C@H]2CCC3=Cc4c(cnn4-c4ccc(F)cc4)C[C@]3(C(=O)c3nccs3)C2)cn1 ; CN([C@H]1CCC2=Cc3c(cnn3-c3ccc(F)cc3)C[C@]2(C(=O)c2cc(C(F)(F)F)ccn2)C1)S(=O)(=O)c1cnnn1C ; CN([C@H]1CCC2=Cc3c(cnn3-c3ccc(F)cc3)C[C@]2(C(=O)c2cc(C(F)(F)F)ccn2)C1)S(=O)(=O)c1cn(C)nn1]

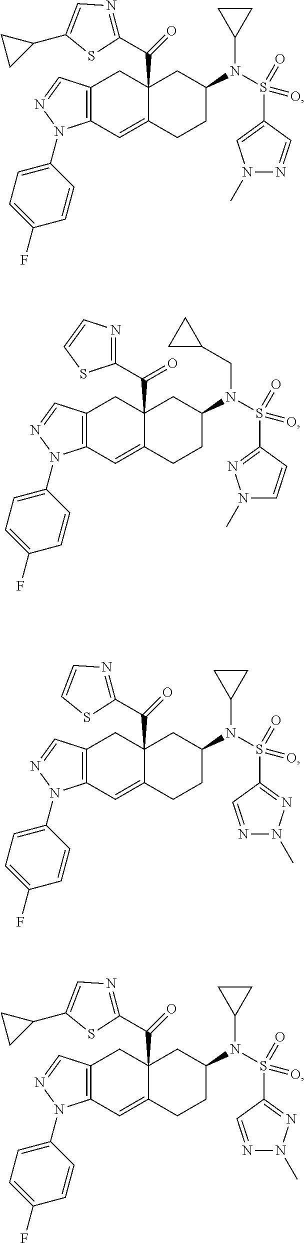 [SMILES: Cn1cc(S(=O)(=O)N(C2CC2)[C@H]2CCC3=Cc4c(cnn4-c4ccc(F)cc4)C[C@]3(C(=O)c3ncc(C4CC4)s3)C2)cn1 ; Cn1ccc(S(=O)(=O)N(CC2CC2)[C@H]2CCC3=Cc4c(cnn4-c4ccc(F)cc4)C[C@]3(C(=O)c3nccs3)C2)n1 ; Cn1ncc(S(=O)(=O)N(C2CC2)[C@H]2CCC3=Cc4c(cnn4-c4ccc(F)cc4)C[C@]3(C(=O)c3nccs3)C2)n1 ; Cn1ncc(S(=O)(=O)N(C2CC2)[C@H]2CCC3=Cc4c(cnn4-c4ccc(F)cc4)C[C@]3(C(=O)c3ncc(C4CC4)s3)C2)n1]

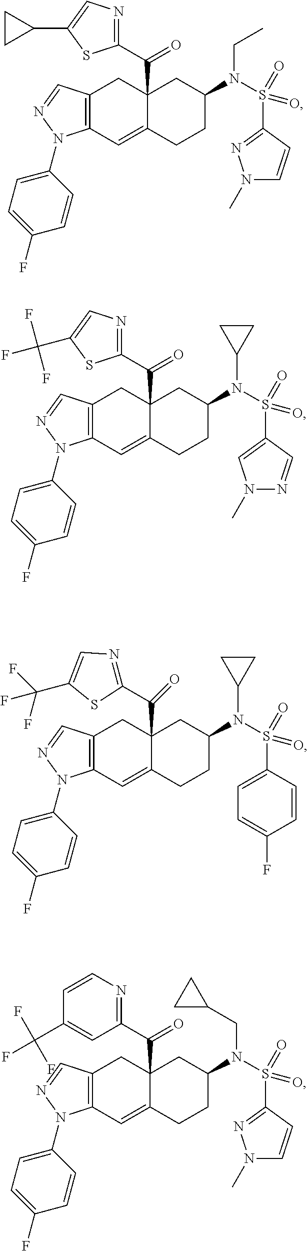 [SMILES: CCN([C@H]1CCC2=Cc3c(cnn3-c3ccc(F)cc3)C[C@]2(C(=O)c2ncc(C3CC3)s2)C1)S(=O)(=O)c1ccn(C)n1 ; Cn1cc(S(=O)(=O)N(C2CC2)[C@H]2CCC3=Cc4c(cnn4-c4ccc(F)cc4)C[C@]3(C(=O)c3ncc(C(F)(F)F)s3)C2)cn1 ; O=C(c1ncc(C(F)(F)F)s1)[C@]12Cc3cnn(-c4ccc(F)cc4)c3C=C1CC[C@H](N(C1CC1)S(=O)(=O)c1ccc(F)cc1)C2 ; Cn1ccc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1]

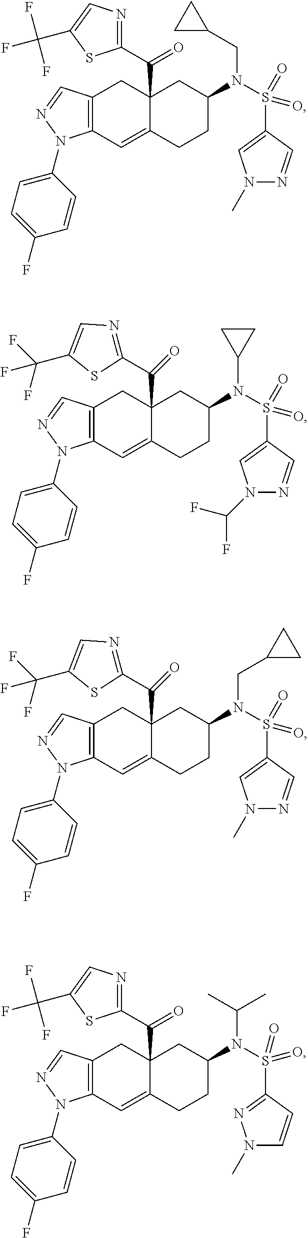 [SMILES: Cn1cc(S(=O)(=O)N(CC2CC2)[C@H]2CCC3=Cc4c(cnn4-c4ccc(F)cc4)C[C@]3(C(=O)c3ncc(C(F)(F)F)s3)C2)cn1 ; O=C(c1ncc(C(F)(F)F)s1)[C@]12Cc3cnn(-c4ccc(F)cc4)c3C=C1CC[C@H](N(C1CC1)S(=O)(=O)c1cnn(C(F)F)c1)C2 ; Cn1cc(S(=O)(=O)N(CC2CC2)[C@H]2CCC3=Cc4c(cnn4-c4ccc(F)cc4)C[C@]3(C(=O)c3ncc(C(F)(F)F)s3)C2)cn1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccn(C)n1]

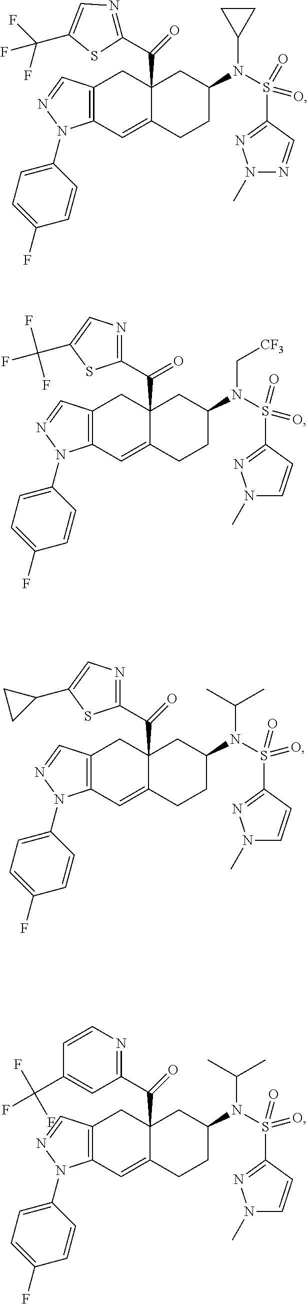 [SMILES: Cn1ncc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1 ; CC(C)N([C@H]1CCC2=Cc3c(cnn3-c3ccc(F)cc3)C[C@]2(C(=O)c2ncc(C3CC3)s2)C1)S(=O)(=O)c1ccn(C)n1 ; CC(C)N([C@H]1CCC2=Cc3c(cnn3-c3ccc(F)cc3)C[C@]2(C(=O)c2cc(C(F)(F)F)ccn2)C1)S(=O)(=O)c1ccn(C)n1]

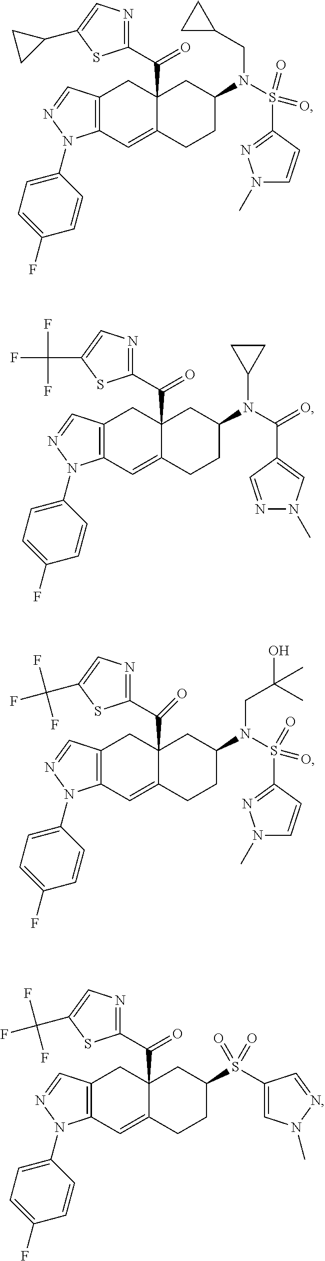 [SMILES: Cn1ccc(S(=O)(=O)N(CC2CC2)[C@H]2CCC3=Cc4c(cnn4-c4ccc(F)cc4)C[C@]3(C(=O)c3ncc(C4CC4)s3)C2)n1 ; Cn1cc(C(=O)N(C2CC2)[C@H]2CCC3=Cc4c(cnn4-c4ccc(F)cc4)C[C@]3(C(=O)c3ncc(C(F)(F)F)s3)C2)cn1 ; Cn1ccc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1 ; Cn1cc(S(=O)(=O)[C@H]2CCC3=Cc4c(cnn4-c4ccc(F)cc4)C[C@]3(C(=O)c3ncc(C(F)(F)F)s3)C2)cn1]

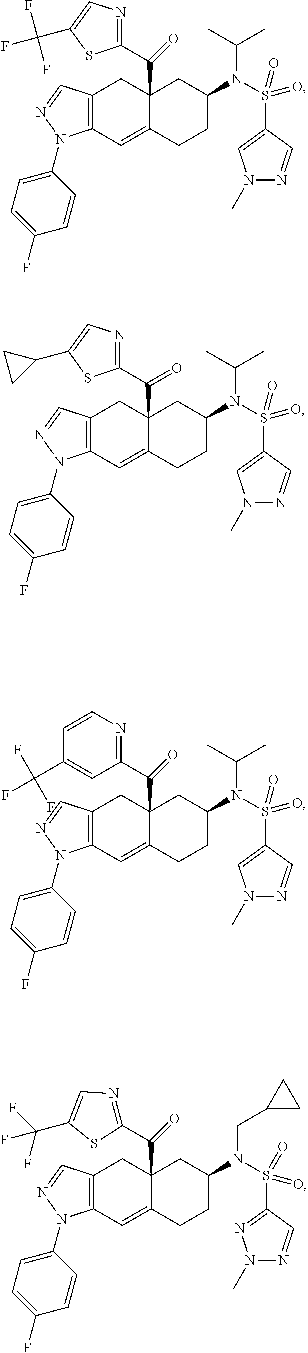 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)c1 ; CC(C)N([C@H]1CCC2=Cc3c(cnn3-c3ccc(F)cc3)C[C@]2(C(=O)c2ncc(C3CC3)s2)C1)S(=O)(=O)c1cnn(C)c1 ; CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; Cn1ncc(S(=O)(=O)N(CC2CC2)[C@H]2CCC3=Cc4c(cnn4-c4ccc(F)cc4)C[C@]3(C(=O)c3ncc(C(F)(F)F)s3)C2)n1]

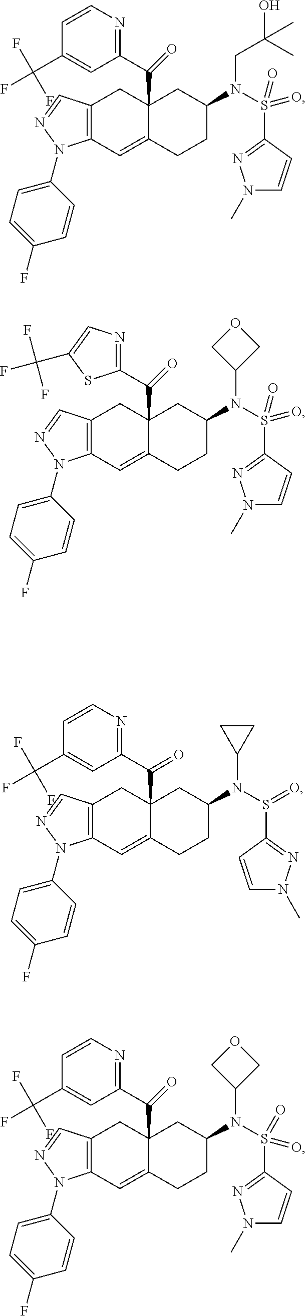 [SMILES: Cn1ccc(S(=O)(=O)N(CC(C)(C)O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(C2COC2)[C@H]2CCC3=Cc4c(cnn4-c4ccc(F)cc4)C[C@]3(C(=O)c3ncc(C(F)(F)F)s3)C2)n1 ; Cn1ccc(S(=O)N(C2CC2)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(C2COC2)[C@H]2CCC3=Cc4c(cnn4-c4ccc(F)cc4)C[C@]3(C(=O)c3cc(C(F)(F)F)ccn3)C2)n1]

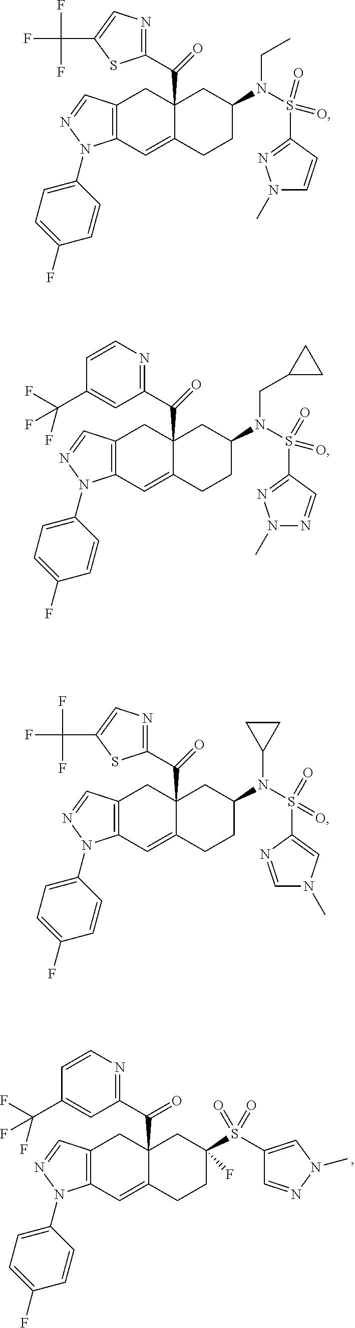 [SMILES: CCN([C@H]1CCC2=Cc3c(cnn3-c3ccc(F)cc3)C[C@]2(C(=O)c2ncc(C(F)(F)F)s2)C1)S(=O)(=O)c1ccn(C)n1 ; Cn1ncc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)c1 ; Cn1cc(S(=O)(=O)[C@@]2(F)CCC3=Cc4c(cnn4-c4ccc(F)cc4)C[C@]3(C(=O)c3cc(C(F)(F)F)ccn3)C2)cn1]

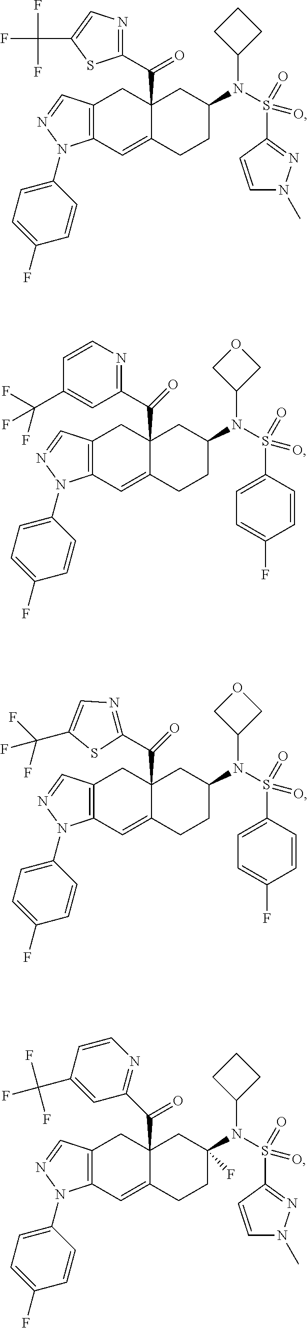 [SMILES: Cn1ccc(S(=O)(=O)N(C2CCC2)[C@H]2CCC3=Cc4c(cnn4-c4ccc(F)cc4)C[C@]3(C(=O)c3ncc(C(F)(F)F)s3)C2)n1 ; O=C(c1cc(C(F)(F)F)ccn1)[C@]12Cc3cnn(-c4ccc(F)cc4)c3C=C1CC[C@H](N(C1COC1)S(=O)(=O)c1ccc(F)cc1)C2 ; O=C(c1ncc(C(F)(F)F)s1)[C@]12Cc3cnn(-c4ccc(F)cc4)c3C=C1CC[C@H](N(C1COC1)S(=O)(=O)c1ccc(F)cc1)C2 ; Cn1ccc(S(=O)(=O)N(C2CCC2)[C@@]2(F)CCC3=Cc4c(cnn4-c4ccc(F)cc4)C[C@]3(C(=O)c3cc(C(F)(F)F)ccn3)C2)n1]

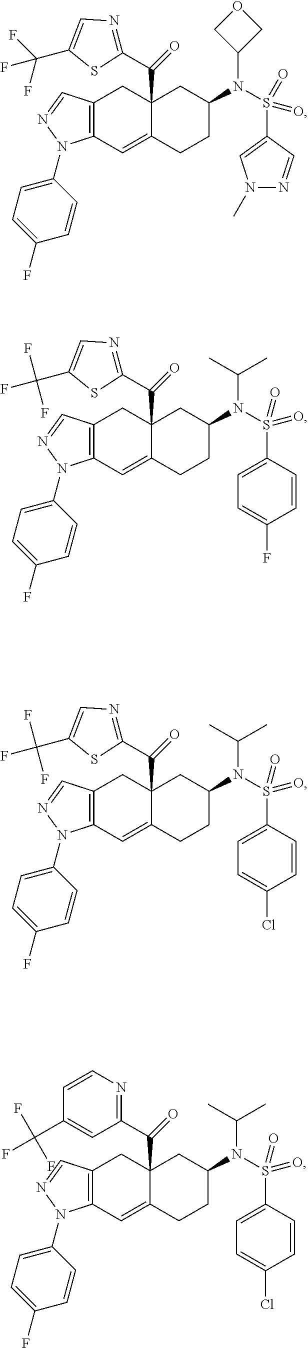 [SMILES: Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)cn1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccc(F)cc1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)cc1 ; CC(C)N([C@H]1CCC2=Cc3c(cnn3-c3ccc(F)cc3)C[C@]2(C(=O)c2cc(C(F)(F)F)ccn2)C1)S(=O)(=O)c1ccc(Cl)cc1]

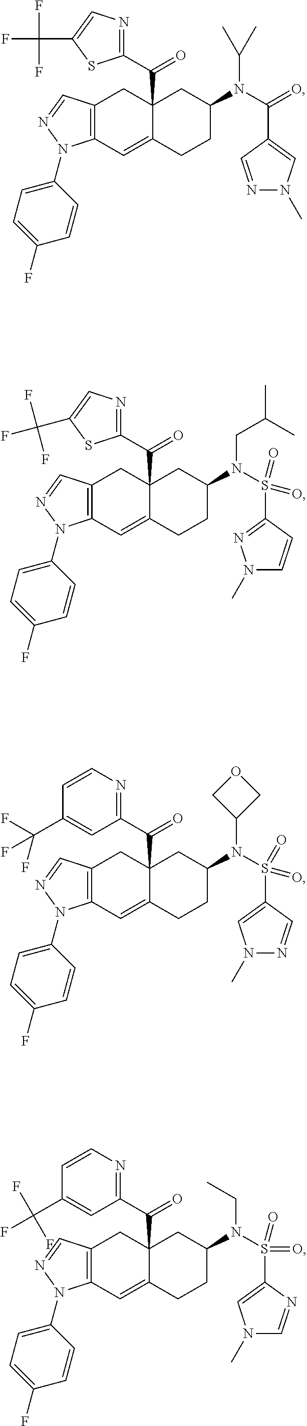 [SMILES: CC(C)N(C(=O)c1cnn(C)c1)[C@H]1CCC2=Cc3c(cnn3-c3ccc(F)cc3)C[C@]2(C(=O)c2ncc(C(F)(F)F)s2)C1 ; CC(C)CN([C@H]1CCC2=Cc3c(cnn3-c3ccc(F)cc3)C[C@]2(C(=O)c2ncc(C(F)(F)F)s2)C1)S(=O)(=O)c1ccn(C)n1 ; Cn1cc(S(=O)(=O)N(C2COC2)[C@H]2CCC3=Cc4c(cnn4-c4ccc(F)cc4)C[C@]3(C(=O)c3cc(C(F)(F)F)ccn3)C2)cn1 ; CCN([C@H]1CCC2=Cc3c(cnn3-c3ccc(F)cc3)C[C@]2(C(=O)c2cc(C(F)(F)F)ccn2)C1)S(=O)(=O)c1cn(C)cn1]

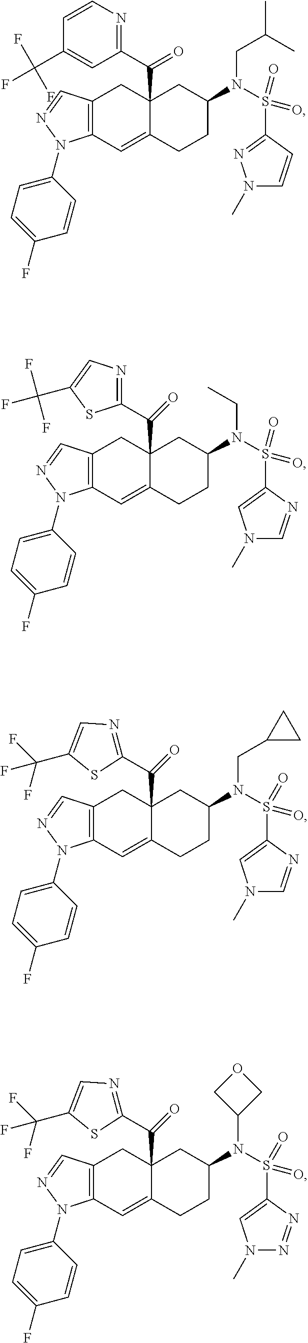 [SMILES: CC(C)CN([C@H]1CCC2=Cc3c(cnn3-c3ccc(F)cc3)C[C@]2(C(=O)c2cc(C(F)(F)F)ccn2)C1)S(=O)(=O)c1ccn(C)n1 ; CCN([C@H]1CCC2=Cc3c(cnn3-c3ccc(F)cc3)C[C@]2(C(=O)c2ncc(C(F)(F)F)s2)C1)S(=O)(=O)c1cn(C)cn1 ; Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)c1 ; Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)nn1]

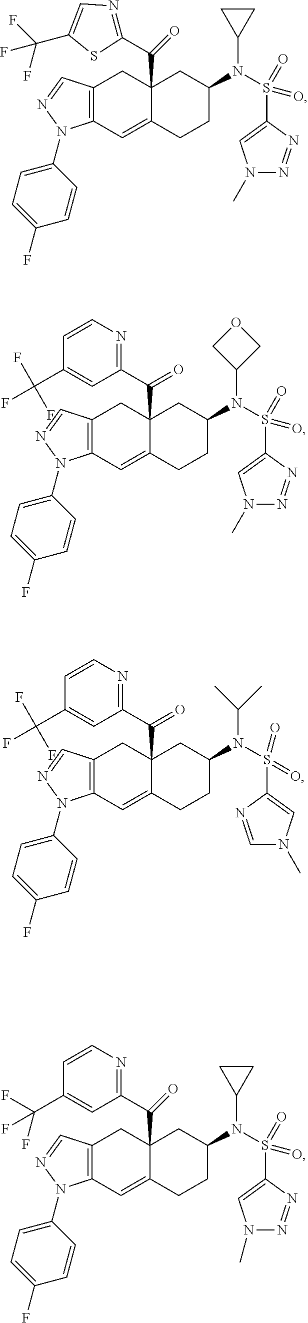 [SMILES: Cn1cc(S(=O)(=O)N(C2CC2)[C@H]2CCC3=Cc4c(cnn4-c4ccc(F)cc4)C[C@]3(C(=O)c3ncc(C(F)(F)F)s3)C2)nn1 ; Cn1cc(S(=O)(=O)N(C2COC2)[C@H]2CCC3=Cc4c(cnn4-c4ccc(F)cc4)C[C@]3(C(=O)c3cc(C(F)(F)F)ccn3)C2)nn1 ; CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)cn1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)nn1]

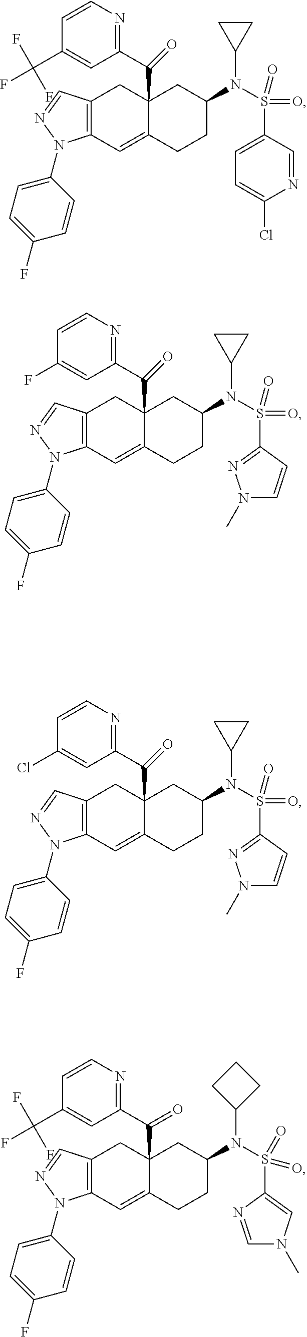 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(Cl)nc1)C2 ; Cn1ccc(S(=O)(=O)N(C2CC2)[C@H]2CCC3=Cc4c(cnn4-c4ccc(F)cc4)C[C@]3(C(=O)c3cc(F)ccn3)C2)n1 ; Cn1ccc(S(=O)(=O)N(C2CC2)[C@H]2CCC3=Cc4c(cnn4-c4ccc(F)cc4)C[C@]3(C(=O)c3cc(Cl)ccn3)C2)n1 ; Cn1cnc(S(=O)(=O)N(C2CCC2)[C@H]2CCC3=Cc4c(cnn4-c4ccc(F)cc4)C[C@]3(C(=O)c3cc(C(F)(F)F)ccn3)C2)c1]

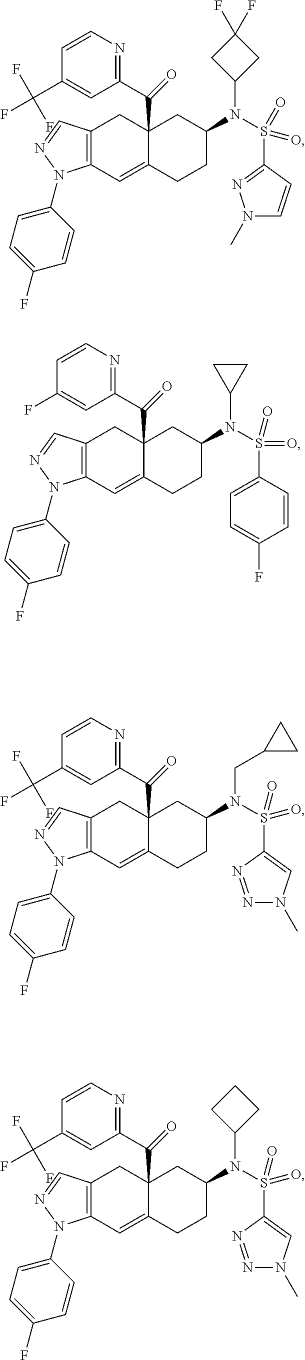 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1 ; O=C(c1cc(F)ccn1)[C@]12Cc3cnn(-c4ccc(F)cc4)c3C=C1CC[C@H](N(C1CC1)S(=O)(=O)c1ccc(F)cc1)C2 ; Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)nn1 ; Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)nn1]

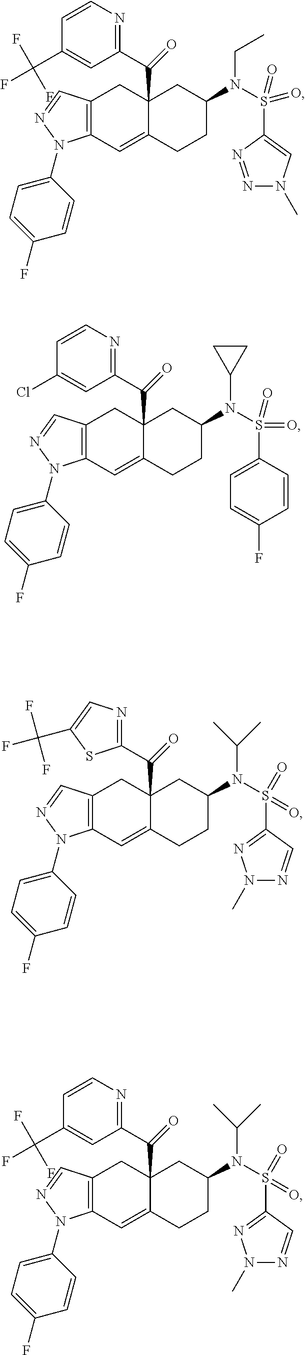 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cn(C)nn1 ; O=C(c1cc(Cl)ccn1)[C@]12Cc3cnn(-c4ccc(F)cc4)c3C=C1CC[C@H](N(C1CC1)S(=O)(=O)c1ccc(F)cc1)C2 ; CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)n1 ; CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)n1]

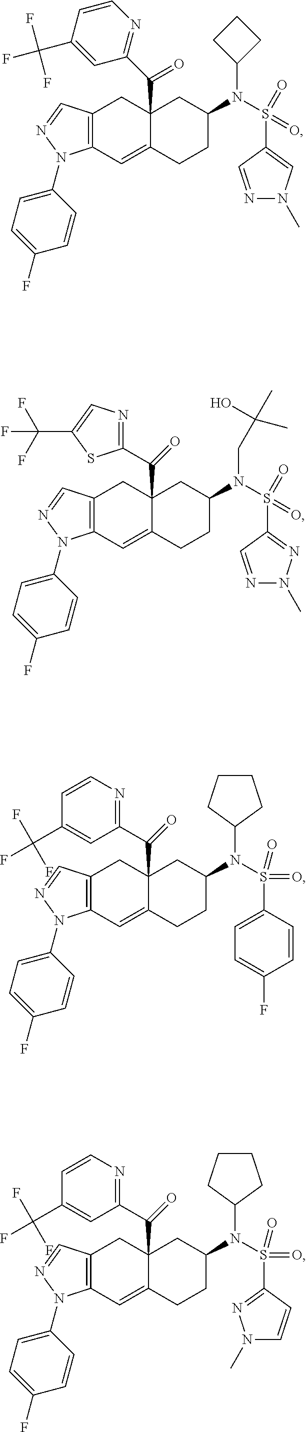 [SMILES: Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)cn1 ; Cn1ncc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1 ; O=C(c1cc(C(F)(F)F)ccn1)[C@]12Cc3cnn(-c4ccc(F)cc4)c3C=C1CC[C@H](N(C1CCCC1)S(=O)(=O)c1ccc(F)cc1)C2 ; Cn1ccc(S(=O)(=O)N(C2CCCC2)[C@H]2CCC3=Cc4c(cnn4-c4ccc(F)cc4)C[C@]3(C(=O)c3cc(C(F)(F)F)ccn3)C2)n1]

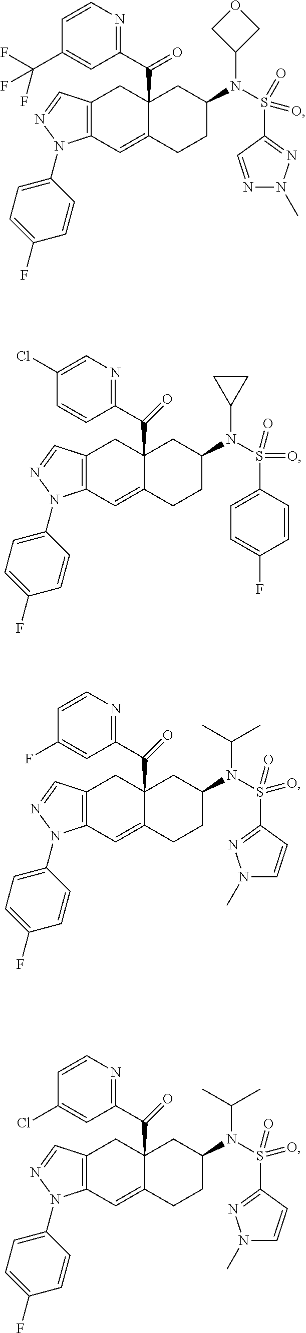 [SMILES: Cn1ncc(S(=O)(=O)N(C2COC2)[C@H]2CCC3=Cc4c(cnn4-c4ccc(F)cc4)C[C@]3(C(=O)c3cc(C(F)(F)F)ccn3)C2)n1 ; O=C(c1ccc(Cl)cn1)[C@]12Cc3cnn(-c4ccc(F)cc4)c3C=C1CC[C@H](N(C1CC1)S(=O)(=O)c1ccc(F)cc1)C2 ; CC(C)N([C@H]1CCC2=Cc3c(cnn3-c3ccc(F)cc3)C[C@]2(C(=O)c2cc(F)ccn2)C1)S(=O)(=O)c1ccn(C)n1 ; CC(C)N([C@H]1CCC2=Cc3c(cnn3-c3ccc(F)cc3)C[C@]2(C(=O)c2cc(Cl)ccn2)C1)S(=O)(=O)c1ccn(C)n1]

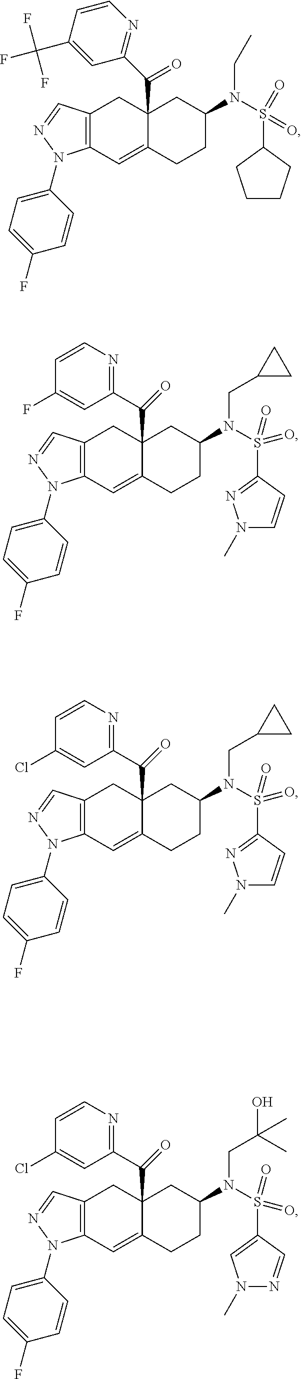 [SMILES: CCN([C@H]1CCC2=Cc3c(cnn3-c3ccc(F)cc3)C[C@]2(C(=O)c2cc(C(F)(F)F)ccn2)C1)S(=O)(=O)C1CCCC1 ; Cn1ccc(S(=O)(=O)N(CC2CC2)[C@H]2CCC3=Cc4c(cnn4-c4ccc(F)cc4)C[C@]3(C(=O)c3cc(F)ccn3)C2)n1 ; Cn1ccc(S(=O)(=O)N(CC2CC2)[C@H]2CCC3=Cc4c(cnn4-c4ccc(F)cc4)C[C@]3(C(=O)c3cc(Cl)ccn3)C2)n1 ; Cn1cc(S(=O)(=O)N(CC(C)(C)O)[C@H]2CCC3=Cc4c(cnn4-c4ccc(F)cc4)C[C@]3(C(=O)c3cc(Cl)ccn3)C2)cn1]

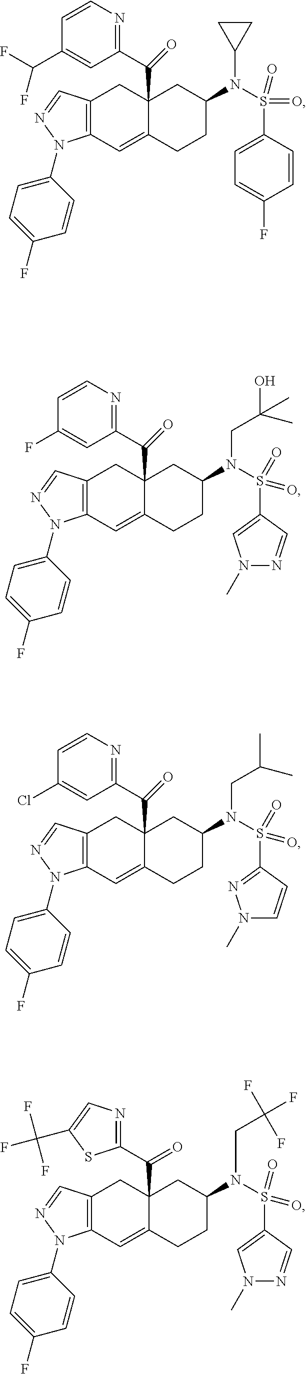 [SMILES: O=C(c1cc(C(F)F)ccn1)[C@]12Cc3cnn(-c4ccc(F)cc4)c3C=C1CC[C@H](N(C1CC1)S(=O)(=O)c1ccc(F)cc1)C2 ; Cn1cc(S(=O)(=O)N(CC(C)(C)O)[C@H]2CCC3=Cc4c(cnn4-c4ccc(F)cc4)C[C@]3(C(=O)c3cc(F)ccn3)C2)cn1 ; CC(C)CN([C@H]1CCC2=Cc3c(cnn3-c3ccc(F)cc3)C[C@]2(C(=O)c2cc(Cl)ccn2)C1)S(=O)(=O)c1ccn(C)n1 ; Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)cn1]

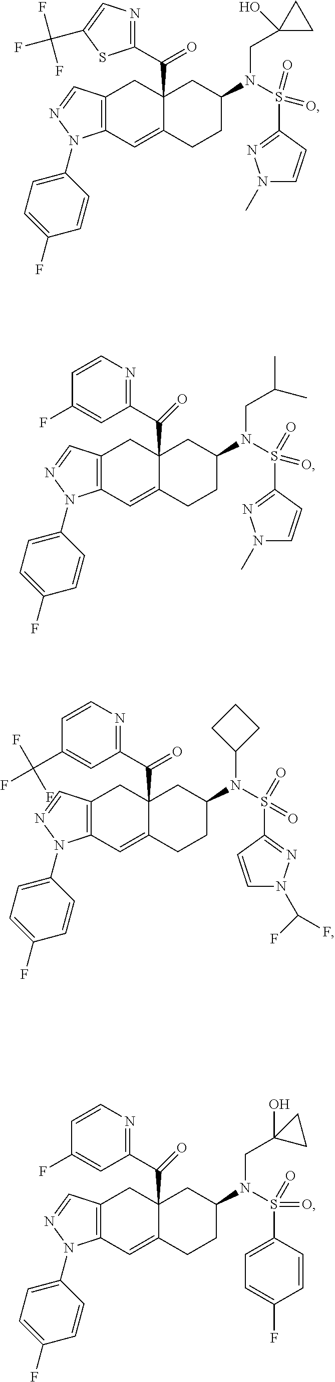 [SMILES: Cn1ccc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1 ; CC(C)CN([C@H]1CCC2=Cc3c(cnn3-c3ccc(F)cc3)C[C@]2(C(=O)c2cc(F)ccn2)C1)S(=O)(=O)c1ccn(C)n1 ; O=C(c1cc(C(F)(F)F)ccn1)[C@]12Cc3cnn(-c4ccc(F)cc4)c3C=C1CC[C@H](N(C1CCC1)S(=O)(=O)c1ccn(C(F)F)n1)C2 ; O=C(c1cc(F)ccn1)[C@]12Cc3cnn(-c4ccc(F)cc4)c3C=C1CC[C@H](N(CC1(O)CC1)S(=O)(=O)c1ccc(F)cc1)C2]

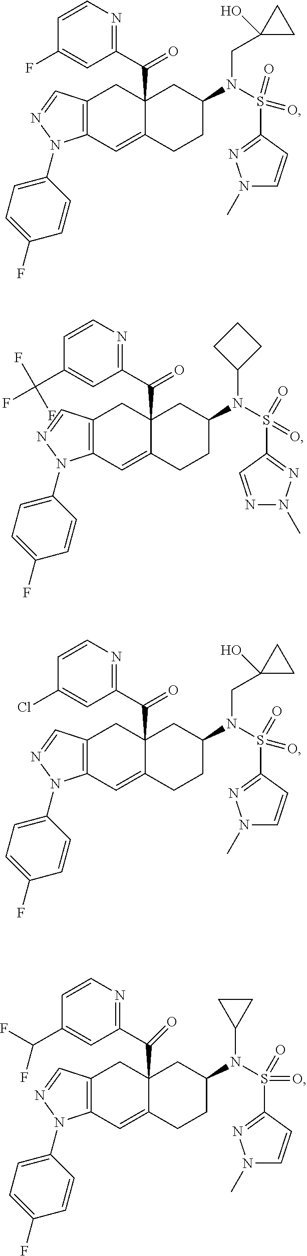 [SMILES: Cn1ccc(S(=O)(=O)N(CC2(O)CC2)[C@H]2CCC3=Cc4c(cnn4-c4ccc(F)cc4)C[C@]3(C(=O)c3cc(F)ccn3)C2)n1 ; Cn1ncc(S(=O)(=O)N(C2CCC2)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CC2(O)CC2)[C@H]2CCC3=Cc4c(cnn4-c4ccc(F)cc4)C[C@]3(C(=O)c3cc(Cl)ccn3)C2)n1 ; Cn1ccc(S(=O)(=O)N(C2CC2)[C@H]2CCC3=Cc4c(cnn4-c4ccc(F)cc4)C[C@]3(C(=O)c3cc(C(F)F)ccn3)C2)n1]

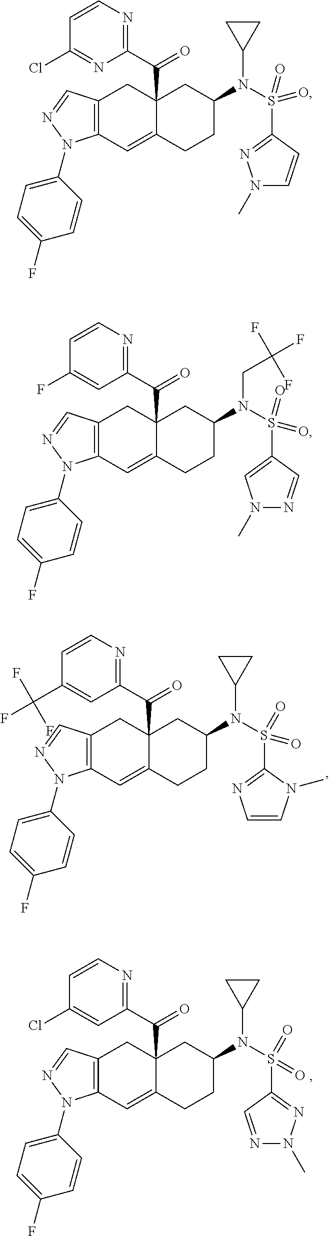 [SMILES: Cn1ccc(S(=O)(=O)N(C2CC2)[C@H]2CCC3=Cc4c(cnn4-c4ccc(F)cc4)C[C@]3(C(=O)c3nccc(Cl)n3)C2)n1 ; Cn1cc(S(=O)(=O)N(CC(F)(F)F)[C@H]2CCC3=Cc4c(cnn4-c4ccc(F)cc4)C[C@]3(C(=O)c3cc(F)ccn3)C2)cn1 ; Cn1ccnc1S(=O)(=O)N(C1CC1)[C@H]1CCC2=Cc3c(cnn3-c3ccc(F)cc3)C[C@]2(C(=O)c2cc(C(F)(F)F)ccn2)C1 ; Cn1ncc(S(=O)(=O)N(C2CC2)[C@H]2CCC3=Cc4c(cnn4-c4ccc(F)cc4)C[C@]3(C(=O)c3cc(Cl)ccn3)C2)n1]

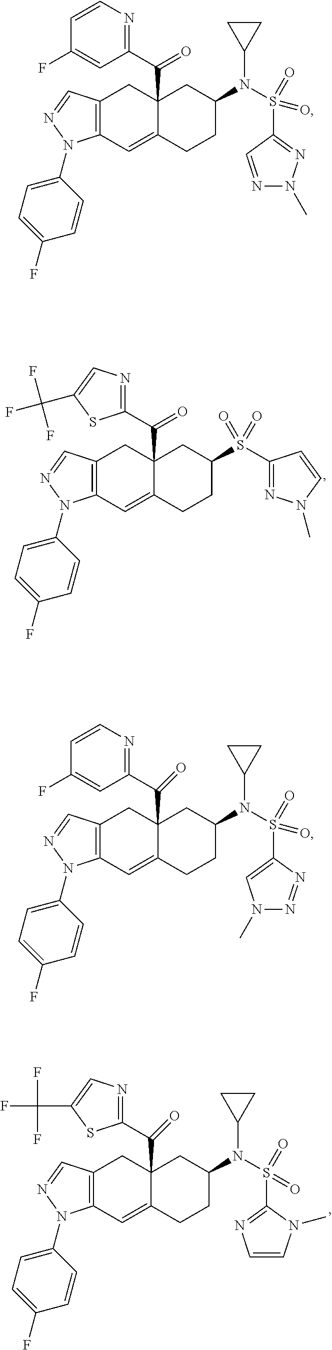 [SMILES: Cn1ncc(S(=O)(=O)N(C2CC2)[C@H]2CCC3=Cc4c(cnn4-c4ccc(F)cc4)C[C@]3(C(=O)c3cc(F)ccn3)C2)n1 ; Cn1ccc(S(=O)(=O)[C@H]2CCC3=Cc4c(cnn4-c4ccc(F)cc4)C[C@]3(C(=O)c3ncc(C(F)(F)F)s3)C2)n1 ; Cn1cc(S(=O)(=O)N(C2CC2)[C@H]2CCC3=Cc4c(cnn4-c4ccc(F)cc4)C[C@]3(C(=O)c3cc(F)ccn3)C2)nn1 ; Cn1ccnc1S(=O)(=O)N(C1CC1)[C@H]1CCC2=Cc3c(cnn3-c3ccc(F)cc3)C[C@]2(C(=O)c2ncc(C(F)(F)F)s2)C1]

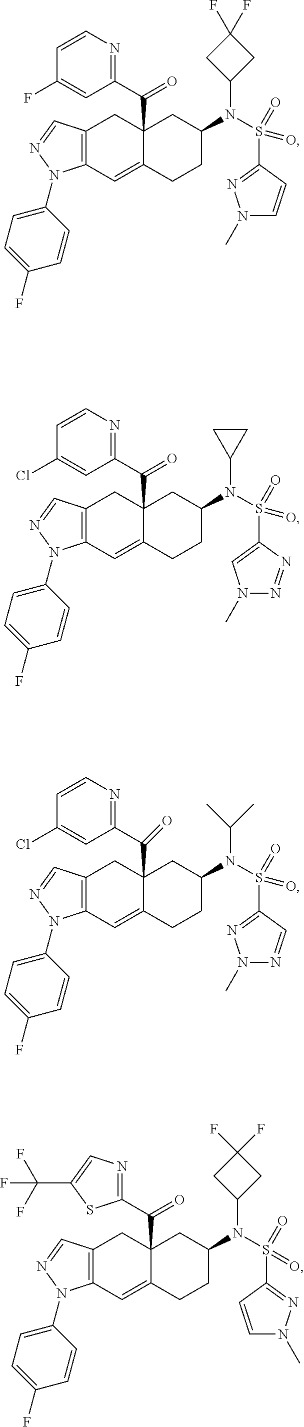 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(F)ccn3)C2)n1 ; Cn1cc(S(=O)(=O)N(C2CC2)[C@H]2CCC3=Cc4c(cnn4-c4ccc(F)cc4)C[C@]3(C(=O)c3cc(Cl)ccn3)C2)nn1 ; CC(C)N([C@H]1CCC2=Cc3c(cnn3-c3ccc(F)cc3)C[C@]2(C(=O)c2cc(Cl)ccn2)C1)S(=O)(=O)c1cnn(C)n1 ; Cn1ccc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1]

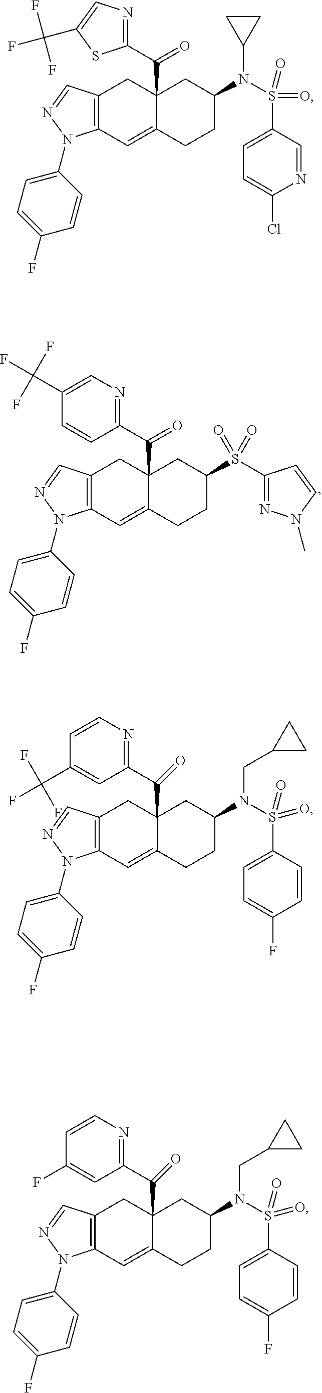 [SMILES: O=C(c1ncc(C(F)(F)F)s1)[C@]12Cc3cnn(-c4ccc(F)cc4)c3C=C1CC[C@H](N(C1CC1)S(=O)(=O)c1ccc(Cl)nc1)C2 ; Cn1ccc(S(=O)(=O)[C@H]2CCC3=Cc4c(cnn4-c4ccc(F)cc4)C[C@]3(C(=O)c3ccc(C(F)(F)F)cn3)C2)n1 ; O=C(c1cc(C(F)(F)F)ccn1)[C@]12Cc3cnn(-c4ccc(F)cc4)c3C=C1CC[C@H](N(CC1CC1)S(=O)(=O)c1ccc(F)cc1)C2 ; O=C(c1cc(F)ccn1)[C@]12Cc3cnn(-c4ccc(F)cc4)c3C=C1CC[C@H](N(CC1CC1)S(=O)(=O)c1ccc(F)cc1)C2]

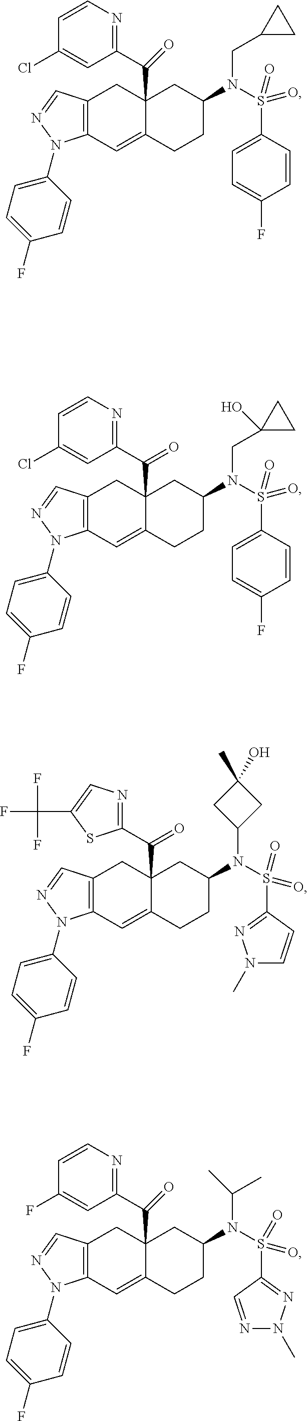

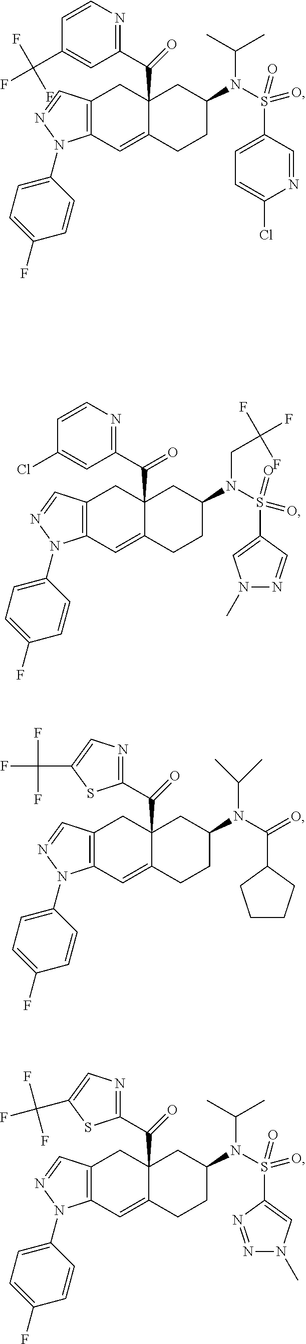 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1 ; Cn1cc(S(=O)(=O)N(CC(F)(F)F)[C@H]2CCC3=Cc4c(cnn4-c4ccc(F)cc4)C[C@]3(C(=O)c3cc(Cl)ccn3)C2)cn1 ; CC(C)N(C(=O)C1CCCC1)[C@H]1CCC2=Cc3c(cnn3-c3ccc(F)cc3)C[C@]2(C(=O)c2ncc(C(F)(F)F)s2)C1 ; CC(C)N([C@H]1CCC2=Cc3c(cnn3-c3ccc(F)cc3)C[C@]2(C(=O)c2ncc(C(F)(F)F)s2)C1)S(=O)(=O)c1cn(C)nn1]

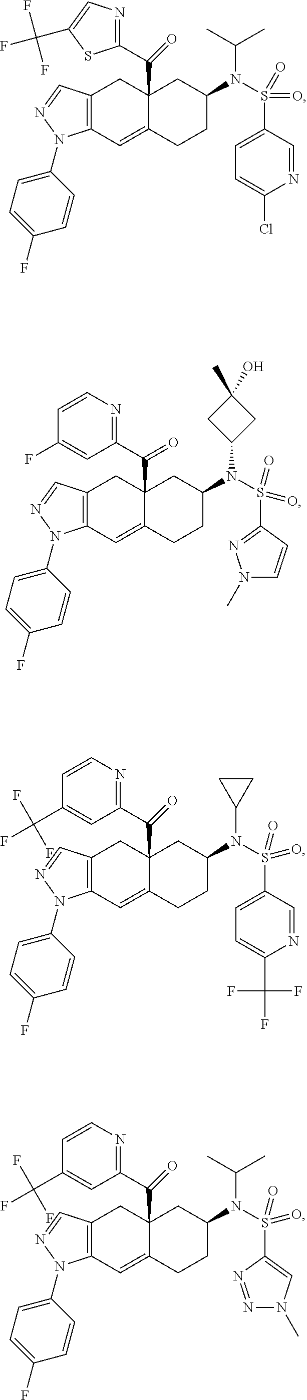 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)nc1 ; Cn1ccc(S(=O)(=O)N([C@H]2CCC3=Cc4c(cnn4-c4ccc(F)cc4)C[C@]3(C(=O)c3cc(F)ccn3)C2)[C@H]2C[C@@](C)(O)C2)n1 ; O=C(c1cc(C(F)(F)F)ccn1)[C@]12Cc3cnn(-c4ccc(F)cc4)c3C=C1CC[C@H](N(C1CC1)S(=O)(=O)c1ccc(C(F)(F)F)nc1)C2 ; CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)nn1]

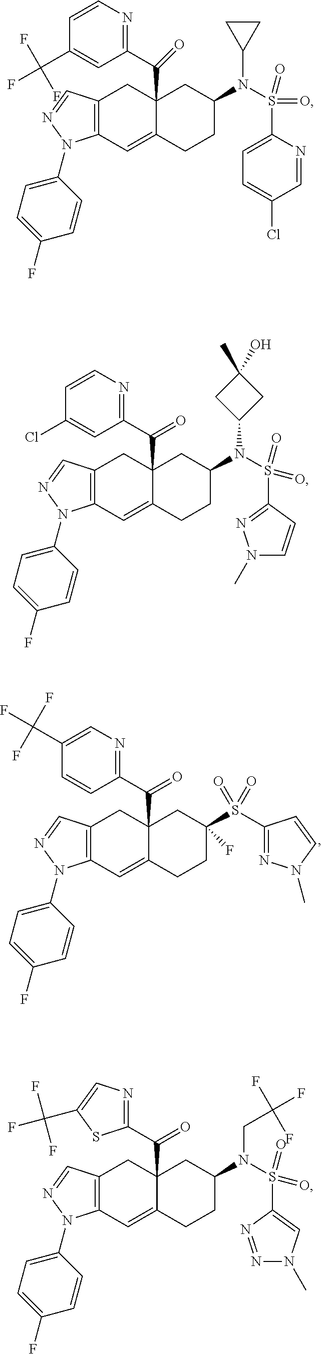 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(Cl)cn1)C2 ; Cn1ccc(S(=O)(=O)N([C@H]2CCC3=Cc4c(cnn4-c4ccc(F)cc4)C[C@]3(C(=O)c3cc(Cl)ccn3)C2)[C@H]2C[C@@](C)(O)C2)n1 ; Cn1ccc(S(=O)(=O)[C@@]2(F)CCC3=Cc4c(cnn4-c4ccc(F)cc4)C[C@]3(C(=O)c3ccc(C(F)(F)F)cn3)C2)n1 ; Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)nn1]

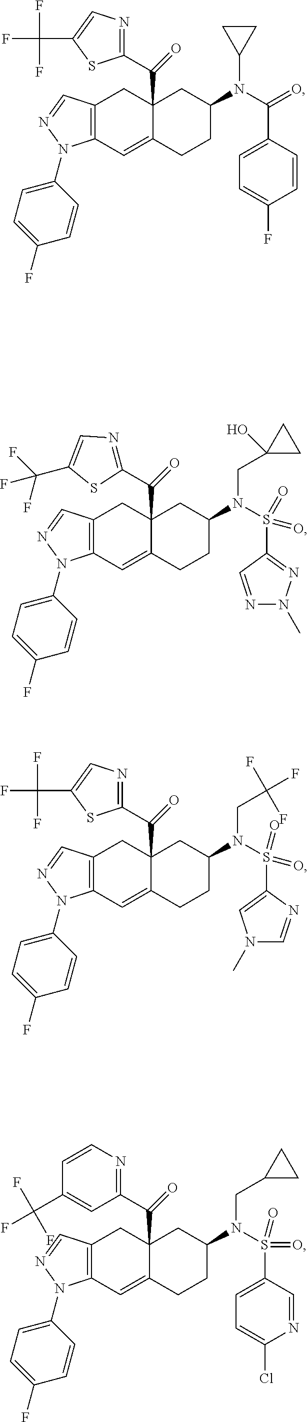 [SMILES: O=C(c1ccc(F)cc1)N(C1CC1)[C@H]1CCC2=Cc3c(cnn3-c3ccc(F)cc3)C[C@]2(C(=O)c2ncc(C(F)(F)F)s2)C1 ; Cn1ncc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)c1 ; O=C(c1cc(C(F)(F)F)ccn1)[C@]12Cc3cnn(-c4ccc(F)cc4)c3C=C1CC[C@H](N(CC1CC1)S(=O)(=O)c1ccc(Cl)nc1)C2]

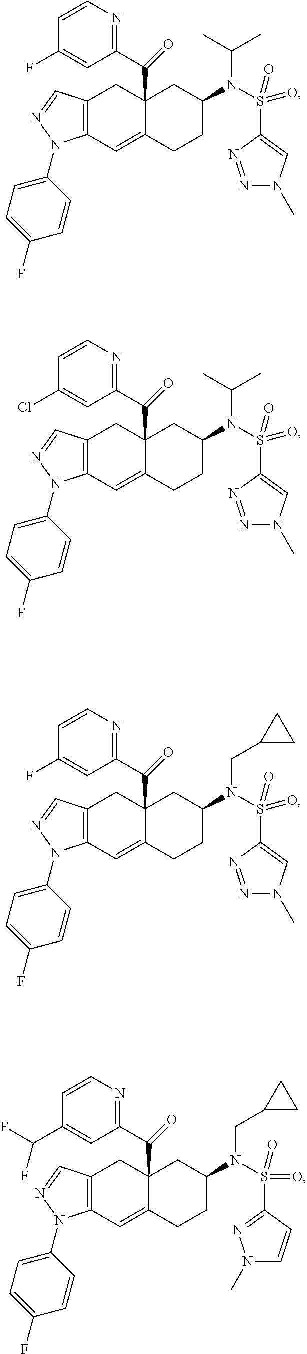 [SMILES: CC(C)N([C@H]1CCC2=Cc3c(cnn3-c3ccc(F)cc3)C[C@]2(C(=O)c2cc(F)ccn2)C1)S(=O)(=O)c1cn(C)nn1 ; CC(C)N([C@H]1CCC2=Cc3c(cnn3-c3ccc(F)cc3)C[C@]2(C(=O)c2cc(Cl)ccn2)C1)S(=O)(=O)c1cn(C)nn1 ; Cn1cc(S(=O)(=O)N(CC2CC2)[C@H]2CCC3=Cc4c(cnn4-c4ccc(F)cc4)C[C@]3(C(=O)c3cc(F)ccn3)C2)nn1 ; Cn1ccc(S(=O)(=O)N(CC2CC2)[C@H]2CCC3=Cc4c(cnn4-c4ccc(F)cc4)C[C@]3(C(=O)c3cc(C(F)F)ccn3)C2)n1]

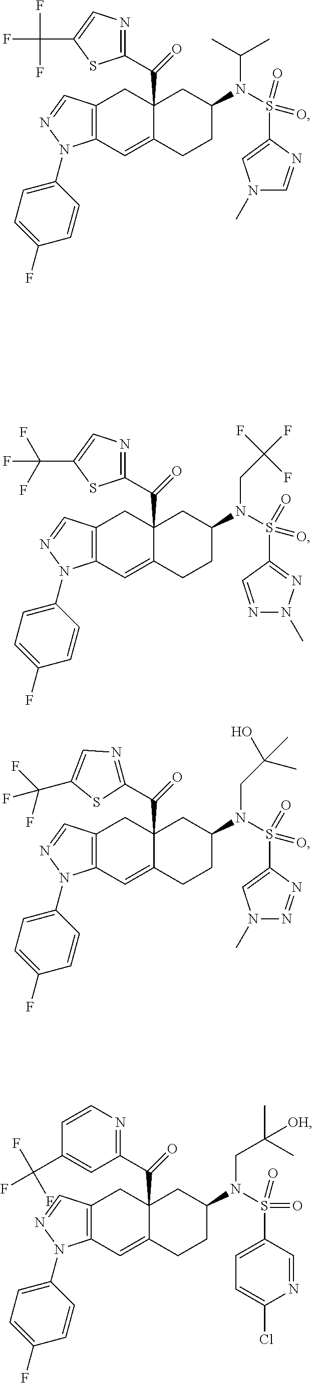 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cn(C)cn1 ; Cn1ncc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1 ; Cn1cc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)nn1 ; CC(C)(O)CN([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1]

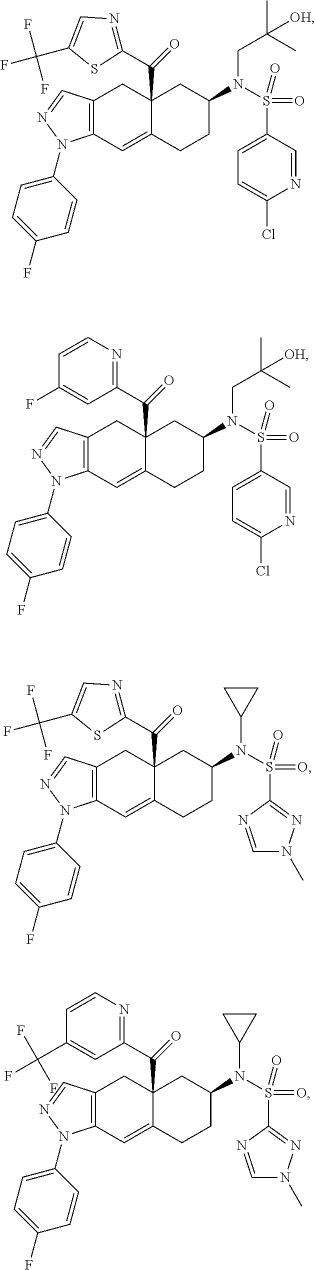 [SMILES: CC(C)(O)CN([C@H]1CCC2=Cc3c(cnn3-c3ccc(F)cc3)C[C@]2(C(=O)c2ncc(C(F)(F)F)s2)C1)S(=O)(=O)c1ccc(Cl)nc1 ; CC(C)(O)CN([C@H]1CCC2=Cc3c(cnn3-c3ccc(F)cc3)C[C@]2(C(=O)c2cc(F)ccn2)C1)S(=O)(=O)c1ccc(Cl)nc1 ; Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(C2CC2)[C@H]2CCC3=Cc4c(cnn4-c4ccc(F)cc4)C[C@]3(C(=O)c3cc(C(F)(F)F)ccn3)C2)n1]

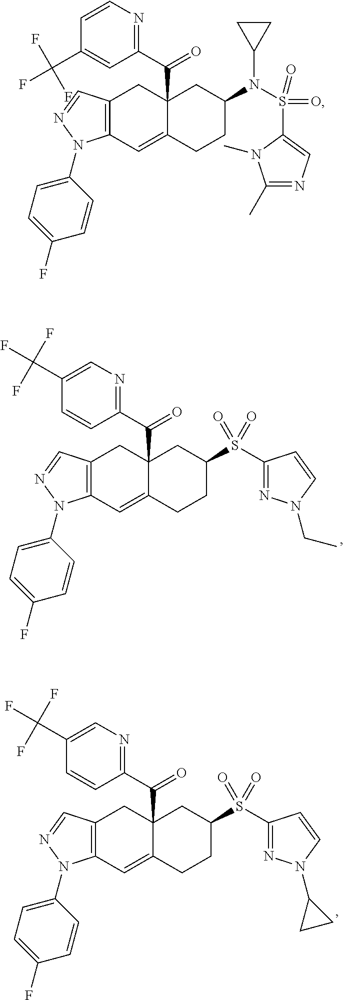 [SMILES: Cc1ncc(S(=O)(=O)N(C2CC2)[C@H]2CCC3=Cc4c(cnn4-c4ccc(F)cc4)C[C@]3(C(=O)c3cc(C(F)(F)F)ccn3)C2)n1C ; CCn1ccc(S(=O)(=O)[C@H]2CCC3=Cc4c(cnn4-c4ccc(F)cc4)C[C@]3(C(=O)c3ccc(C(F)(F)F)cn3)C2)n1 ; O=C(c1ccc(C(F)(F)F)cn1)[C@]12Cc3cnn(-c4ccc(F)cc4)c3C=C1CC[C@H](S(=O)(=O)c1ccn(C3CC3)n1)C2]

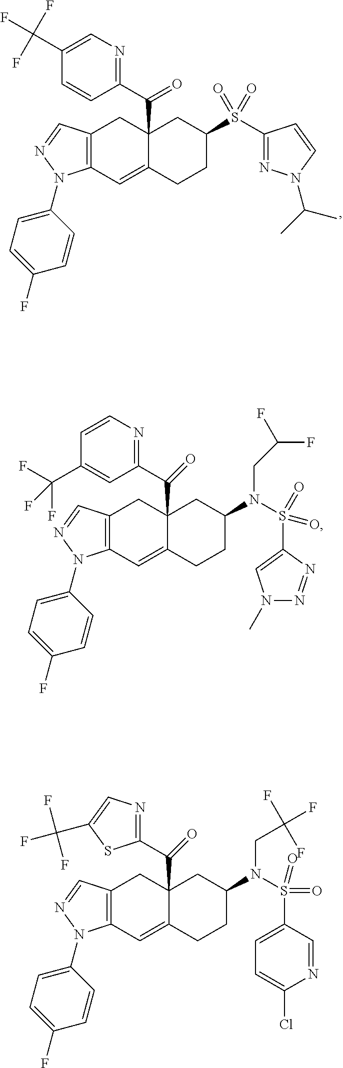 [SMILES: CC(C)n1ccc(S(=O)(=O)[C@H]2CCC3=Cc4c(cnn4-c4ccc(F)cc4)C[C@]3(C(=O)c3ccc(C(F)(F)F)cn3)C2)n1 ; Cn1cc(S(=O)(=O)N(CC(F)F)[C@H]2CCC3=Cc4c(cnn4-c4ccc(F)cc4)C[C@]3(C(=O)c3cc(C(F)(F)F)ccn3)C2)nn1 ; O=C(c1ncc(C(F)(F)F)s1)[C@]12Cc3cnn(-c4ccc(F)cc4)c3C=C1CC[C@H](N(CC(F)(F)F)S(=O)(=O)c1ccc(Cl)nc1)C2]

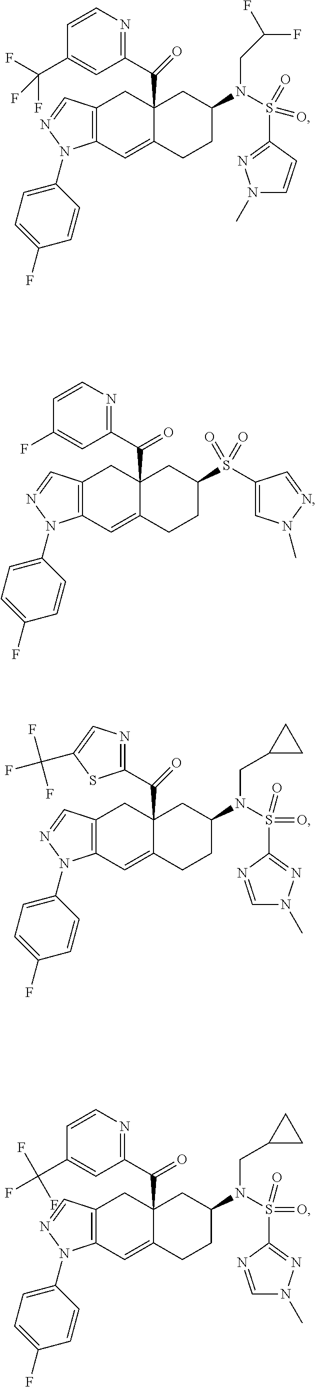 [SMILES: Cn1ccc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1 ; Cn1cc(S(=O)(=O)[C@H]2CCC3=Cc4c(cnn4-c4ccc(F)cc4)C[C@]3(C(=O)c3cc(F)ccn3)C2)cn1 ; Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1]

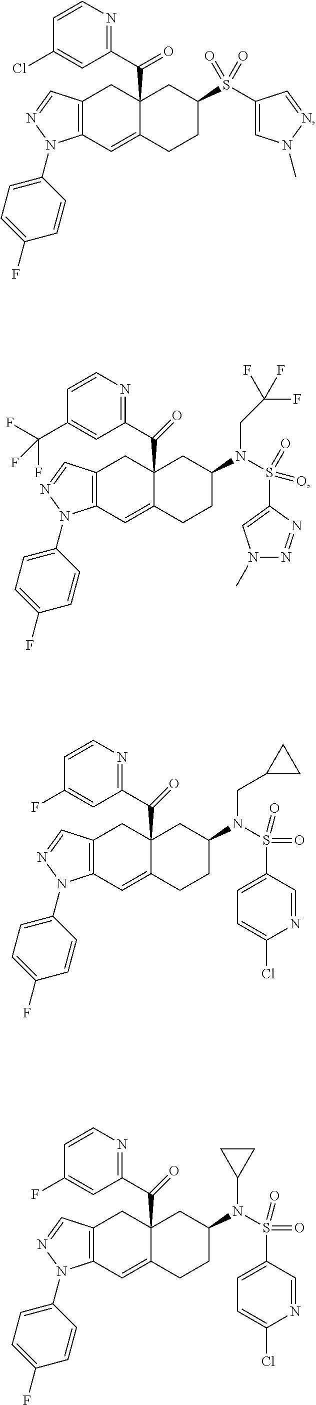 [SMILES: Cn1cc(S(=O)(=O)[C@H]2CCC3=Cc4c(cnn4-c4ccc(F)cc4)C[C@]3(C(=O)c3cc(Cl)ccn3)C2)cn1 ; Cn1cc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)nn1 ; O=C(c1cc(F)ccn1)[C@]12Cc3cnn(-c4ccc(F)cc4)c3C=C1CC[C@H](N(CC1CC1)S(=O)(=O)c1ccc(Cl)nc1)C2 ; O=C(c1cc(F)ccn1)[C@]12Cc3cnn(-c4ccc(F)cc4)c3C=C1CC[C@H](N(C1CC1)S(=O)(=O)c1ccc(Cl)nc1)C2]

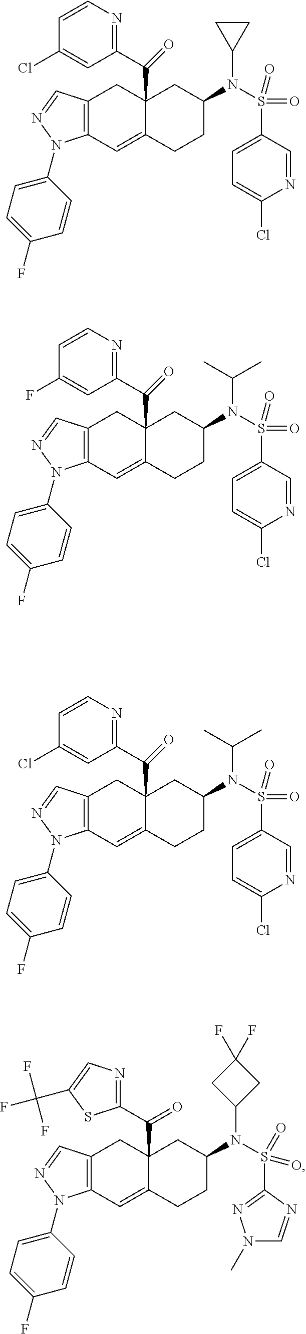 [SMILES: O=C(c1cc(Cl)ccn1)[C@]12Cc3cnn(-c4ccc(F)cc4)c3C=C1CC[C@H](N(C1CC1)S(=O)(=O)c1ccc(Cl)nc1)C2 ; CC(C)N([C@H]1CCC2=Cc3c(cnn3-c3ccc(F)cc3)C[C@]2(C(=O)c2cc(F)ccn2)C1)S(=O)(=O)c1ccc(Cl)nc1 ; CC(C)N([C@H]1CCC2=Cc3c(cnn3-c3ccc(F)cc3)C[C@]2(C(=O)c2cc(Cl)ccn2)C1)S(=O)(=O)c1ccc(Cl)nc1 ; Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1]

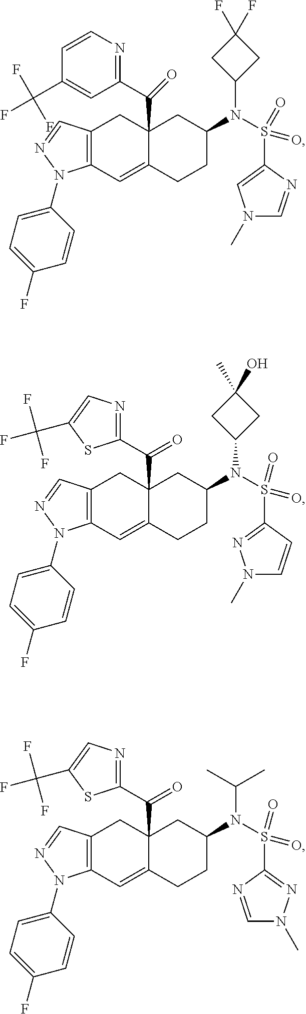 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)c1 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](C)(O)C2)n1 ; CC(C)N([C@H]1CCC2=Cc3c(cnn3-c3ccc(F)cc3)C[C@]2(C(=O)c2ncc(C(F)(F)F)s2)C1)S(=O)(=O)c1ncn(C)n1]

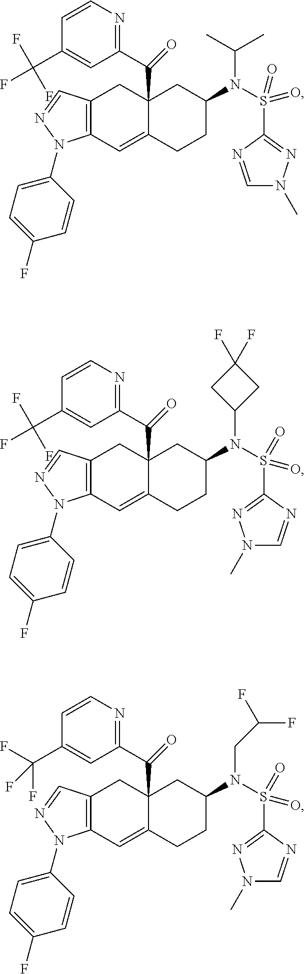 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ncn(C)n1 ; Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1]

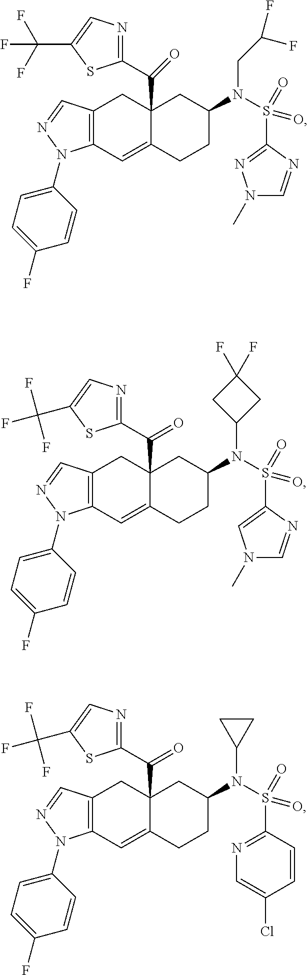 [SMILES: Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)c1 ; O=C(c1ncc(C(F)(F)F)s1)[C@]12Cc3cnn(-c4ccc(F)cc4)c3C=C1CC[C@H](N(C1CC1)S(=O)(=O)c1ccc(Cl)cn1)C2]

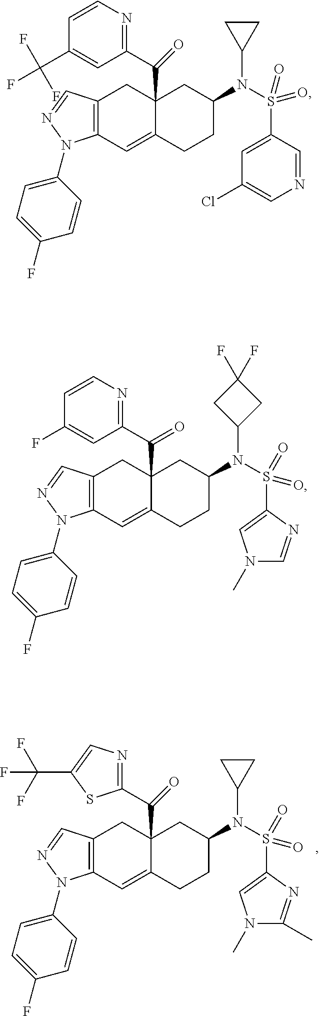 [SMILES: O=C(c1cc(C(F)(F)F)ccn1)[C@]12Cc3cnn(-c4ccc(F)cc4)c3C=C1CC[C@H](N(C1CC1)S(=O)(=O)c1cncc(Cl)c1)C2 ; Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)c1 ; Cc1nc(S(=O)(=O)N(C2CC2)[C@H]2CCC3=Cc4c(cnn4-c4ccc(F)cc4)C[C@]3(C(=O)c3ncc(C(F)(F)F)s3)C2)cn1C]

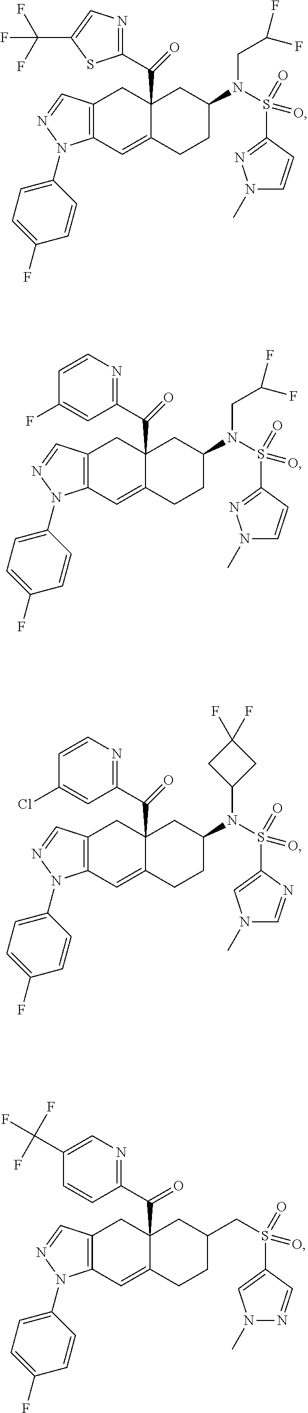 [SMILES: Cn1ccc(S(=O)(=O)N(CC(F)F)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N(CC(F)F)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)c1 ; Cn1cc(S(=O)(=O)CC2CCC3=Cc4c(cnn4-c4ccc(F)cc4)C[C@]3(C(=O)c3ccc(C(F)(F)F)cn3)C2)cn1]

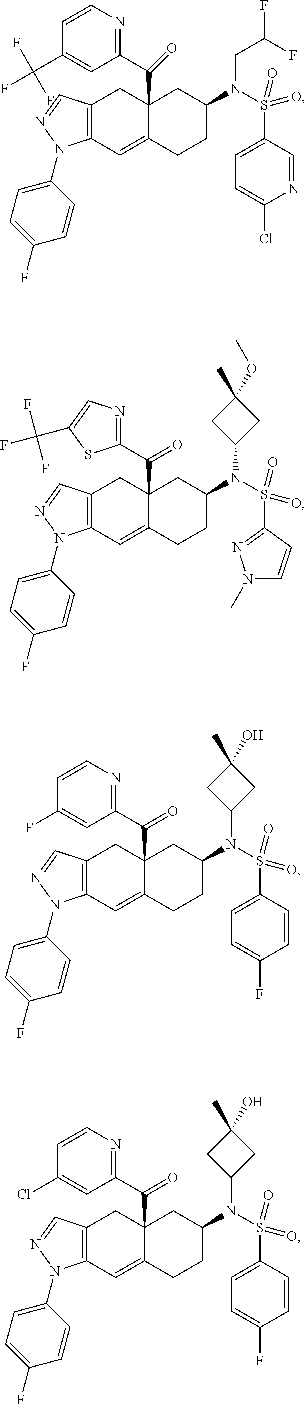

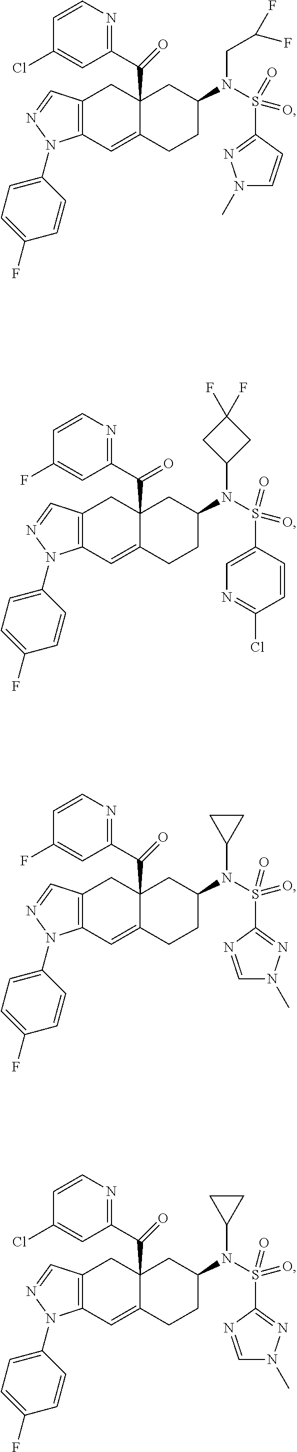 [SMILES: Cn1ccc(S(=O)(=O)N(CC(F)F)[C@H]2CCC3=Cc4c(cnn4-c4ccc(F)cc4)C[C@]3(C(=O)c3cc(Cl)ccn3)C2)n1 ; O=C(c1cc(F)ccn1)[C@]12Cc3cnn(-c4ccc(F)cc4)c3C=C1CC[C@H](N(C1CC(F)(F)C1)S(=O)(=O)c1ccc(Cl)nc1)C2 ; Cn1cnc(S(=O)(=O)N(C2CC2)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC2)[C@H]2CCC3=Cc4c(cnn4-c4ccc(F)cc4)C[C@]3(C(=O)c3cc(Cl)ccn3)C2)n1]

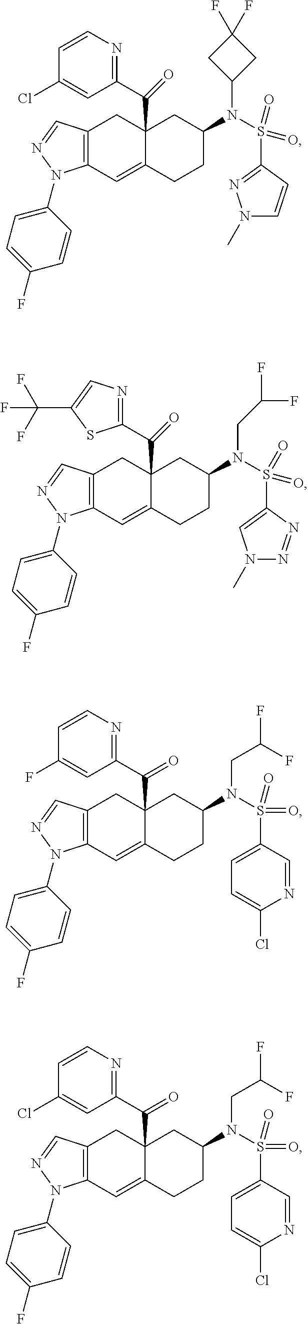 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1 ; Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)nn1 ; O=C(c1cc(F)ccn1)[C@]12Cc3cnn(-c4ccc(F)cc4)c3C=C1CC[C@H](N(CC(F)F)S(=O)(=O)c1ccc(Cl)nc1)C2 ; O=C(c1cc(Cl)ccn1)[C@]12Cc3cnn(-c4ccc(F)cc4)c3C=C1CC[C@H](N(CC(F)F)S(=O)(=O)c1ccc(Cl)nc1)C2]

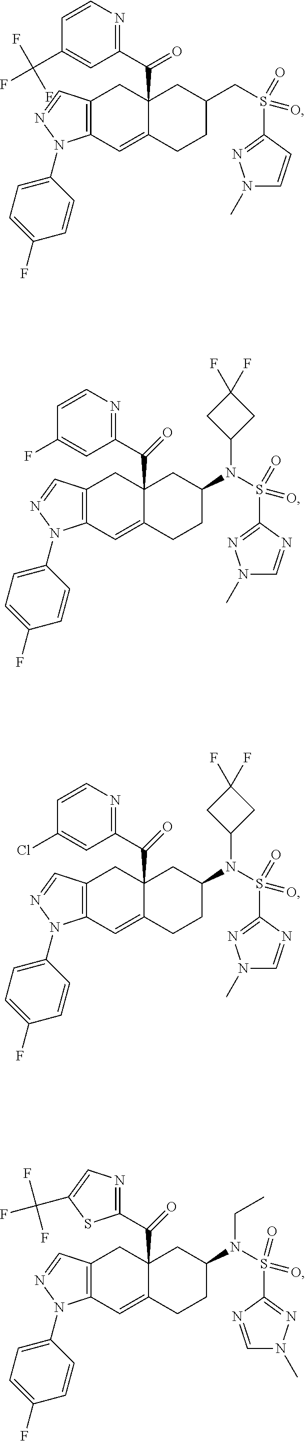 [SMILES: Cn1ccc(S(=O)(=O)CC2CCC3=Cc4c(cnn4-c4ccc(F)cc4)C[C@]3(C(=O)c3cc(C(F)(F)F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1 ; CCN([C@H]1CCC2=Cc3c(cnn3-c3ccc(F)cc3)C[C@]2(C(=O)c2ncc(C(F)(F)F)s2)C1)S(=O)(=O)c1ncn(C)n1]

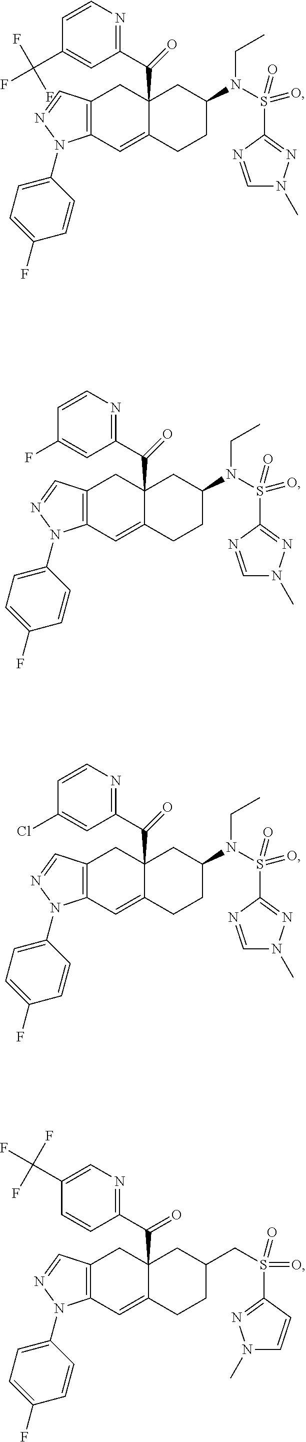 [SMILES: CCN([C@H]1CCC2=Cc3c(cnn3-c3ccc(F)cc3)C[C@]2(C(=O)c2cc(C(F)(F)F)ccn2)C1)S(=O)(=O)c1ncn(C)n1 ; CCN([C@H]1CCC2=Cc3c(cnn3-c3ccc(F)cc3)C[C@]2(C(=O)c2cc(F)ccn2)C1)S(=O)(=O)c1ncn(C)n1 ; CCN([C@H]1CCC2=Cc3c(cnn3-c3ccc(F)cc3)C[C@]2(C(=O)c2cc(Cl)ccn2)C1)S(=O)(=O)c1ncn(C)n1 ; Cn1ccc(S(=O)(=O)CC2CCC3=Cc4c(cnn4-c4ccc(F)cc4)C[C@]3(C(=O)c3ccc(C(F)(F)F)cn3)C2)n1]

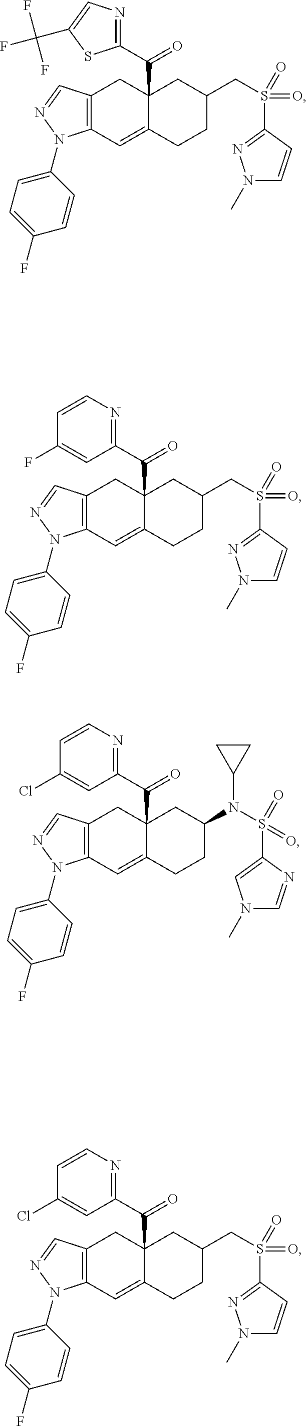 [SMILES: Cn1ccc(S(=O)(=O)CC2CCC3=Cc4c(cnn4-c4ccc(F)cc4)C[C@]3(C(=O)c3ncc(C(F)(F)F)s3)C2)n1 ; Cn1ccc(S(=O)(=O)CC2CCC3=Cc4c(cnn4-c4ccc(F)cc4)C[C@]3(C(=O)c3cc(F)ccn3)C2)n1 ; Cn1cnc(S(=O)(=O)N(C2CC2)[C@H]2CCC3=Cc4c(cnn4-c4ccc(F)cc4)C[C@]3(C(=O)c3cc(Cl)ccn3)C2)c1 ; Cn1ccc(S(=O)(=O)CC2CCC3=Cc4c(cnn4-c4ccc(F)cc4)C[C@]3(C(=O)c3cc(Cl)ccn3)C2)n1]

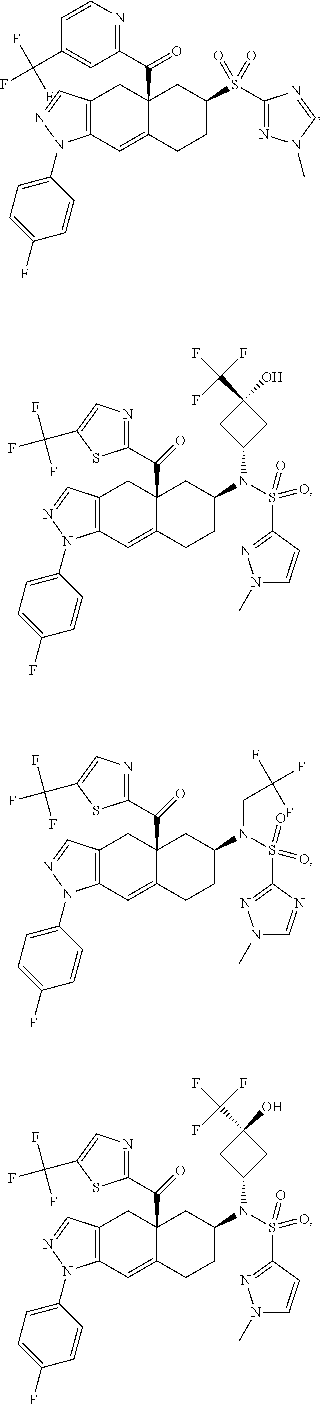 [SMILES: Cn1cnc(S(=O)(=O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](O)(C(F)(F)F)C2)n1 ; Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@](O)(C(F)(F)F)C2)n1]

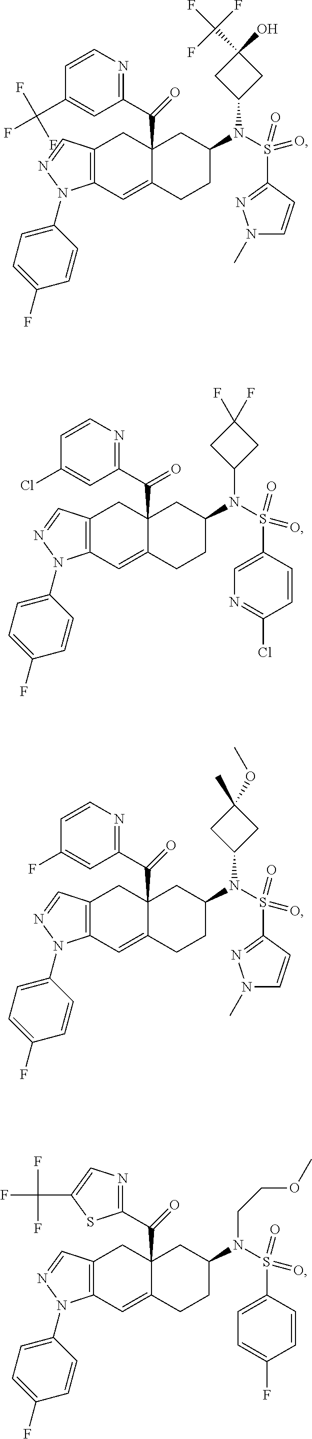 [SMILES: Cn1ccc(S(=O)(=O)N([C@H]2CCC3=Cc4c(cnn4-c4ccc(F)cc4)C[C@]3(C(=O)c3cc(C(F)(F)F)ccn3)C2)[C@H]2C[C@@](O)(C(F)(F)F)C2)n1 ; O=C(c1cc(Cl)ccn1)[C@]12Cc3cnn(-c4ccc(F)cc4)c3C=C1CC[C@H](N(C1CC(F)(F)C1)S(=O)(=O)c1ccc(Cl)nc1)C2 ; CO[C@]1(C)C[C@H](N([C@H]2CCC3=Cc4c(cnn4-c4ccc(F)cc4)C[C@]3(C(=O)c3cc(F)ccn3)C2)S(=O)(=O)c2ccn(C)n2)C1 ; COCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1]

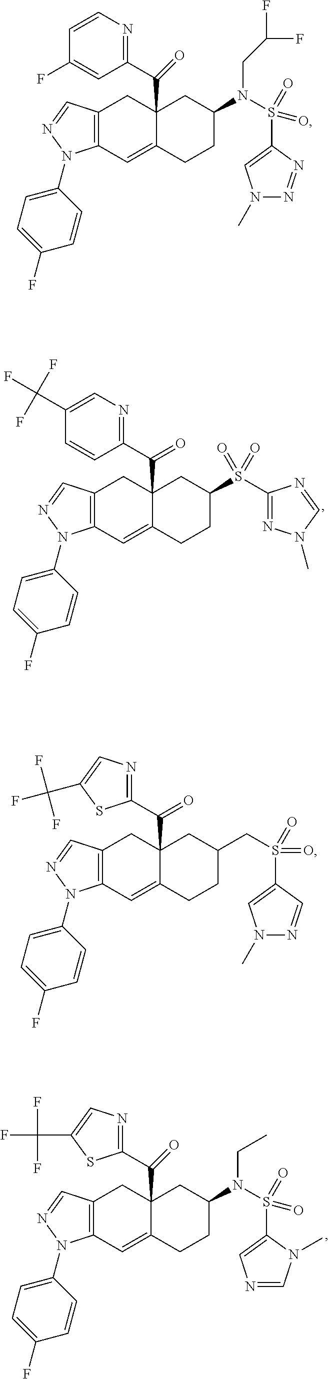 [SMILES: Cn1cc(S(=O)(=O)N(CC(F)F)[C@H]2CCC3=Cc4c(cnn4-c4ccc(F)cc4)C[C@]3(C(=O)c3cc(F)ccn3)C2)nn1 ; Cn1cnc(S(=O)(=O)[C@H]2CCC3=Cc4c(cnn4-c4ccc(F)cc4)C[C@]3(C(=O)c3ccc(C(F)(F)F)cn3)C2)n1 ; Cn1cc(S(=O)(=O)CC2CCC3=Cc4c(cnn4-c4ccc(F)cc4)C[C@]3(C(=O)c3ncc(C(F)(F)F)s3)C2)cn1 ; CCN([C@H]1CCC2=Cc3c(cnn3-c3ccc(F)cc3)C[C@]2(C(=O)c2ncc(C(F)(F)F)s2)C1)S(=O)(=O)c1cncn1C]

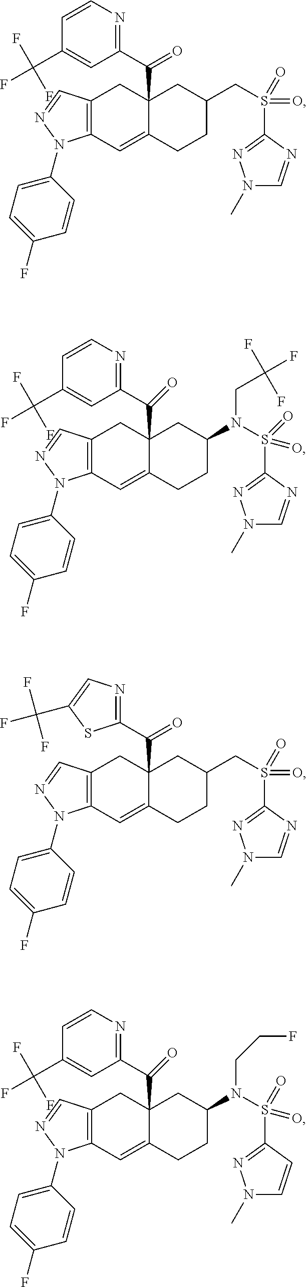 [SMILES: Cn1cnc(S(=O)(=O)CC2CCC3=Cc4c(cnn4-c4ccc(F)cc4)C[C@]3(C(=O)c3cc(C(F)(F)F)ccn3)C2)n1 ; Cn1cnc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)CC2CCC3=Cc4c(cnn4-c4ccc(F)cc4)C[C@]3(C(=O)c3ncc(C(F)(F)F)s3)C2)n1 ; Cn1ccc(S(=O)(=O)N(CCF)[C@H]2CCC3=Cc4c(cnn4-c4ccc(F)cc4)C[C@]3(C(=O)c3cc(C(F)(F)F)ccn3)C2)n1]

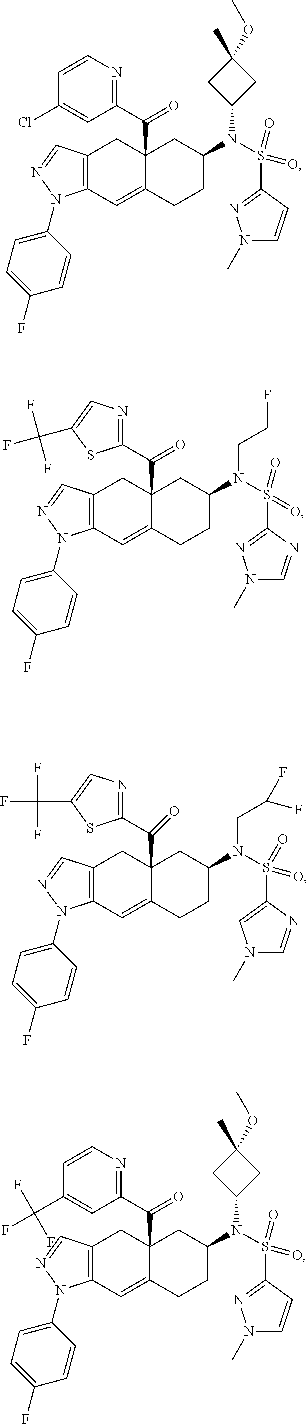 [SMILES: CO[C@]1(C)C[C@H](N([C@H]2CCC3=Cc4c(cnn4-c4ccc(F)cc4)C[C@]3(C(=O)c3cc(Cl)ccn3)C2)S(=O)(=O)c2ccn(C)n2)C1 ; Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)c1 ; CO[C@]1(C)C[C@H](N([C@H]2CCC3=Cc4c(cnn4-c4ccc(F)cc4)C[C@]3(C(=O)c3cc(C(F)(F)F)ccn3)C2)S(=O)(=O)c2ccn(C)n2)C1]

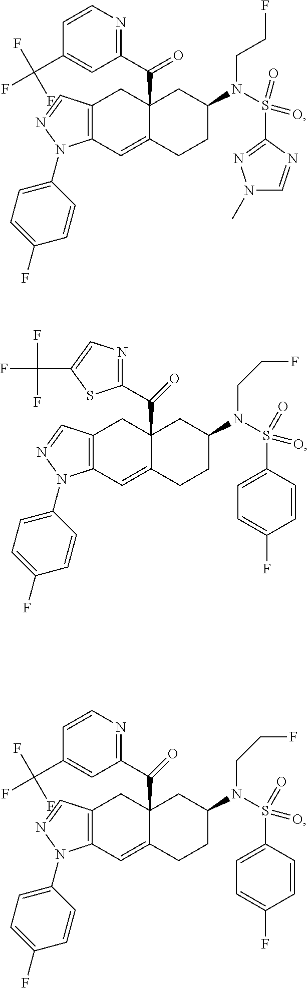 [SMILES: Cn1cnc(S(=O)(=O)N(CCF)[C@H]2CCC3=Cc4c(cnn4-c4ccc(F)cc4)C[C@]3(C(=O)c3cc(C(F)(F)F)ccn3)C2)n1 ; O=C(c1ncc(C(F)(F)F)s1)[C@]12Cc3cnn(-c4ccc(F)cc4)c3C=C1CC[C@H](N(CCF)S(=O)(=O)c1ccc(F)cc1)C2 ; O=C(c1cc(C(F)(F)F)ccn1)[C@]12Cc3cnn(-c4ccc(F)cc4)c3C=C1CC[C@H](N(CCF)S(=O)(=O)c1ccc(F)cc1)C2]

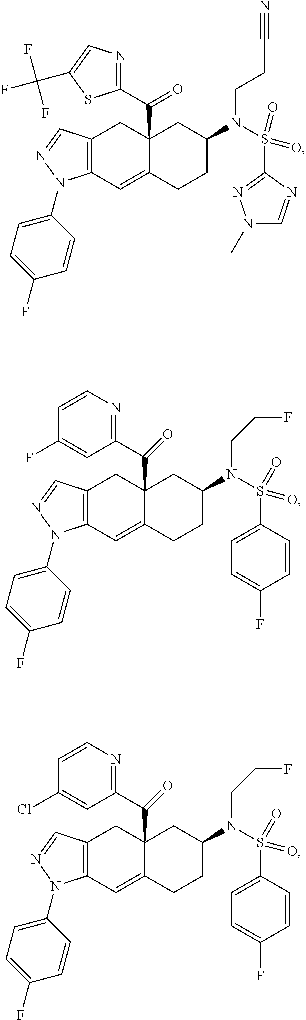 [SMILES: Cn1cnc(S(=O)(=O)N(CCC#N)[C@H]2CCC3=Cc4c(cnn4-c4ccc(F)cc4)C[C@]3(C(=O)c3ncc(C(F)(F)F)s3)C2)n1 ; O=C(c1cc(F)ccn1)[C@]12Cc3cnn(-c4ccc(F)cc4)c3C=C1CC[C@H](N(CCF)S(=O)(=O)c1ccc(F)cc1)C2 ; O=C(c1cc(Cl)ccn1)[C@]12Cc3cnn(-c4ccc(F)cc4)c3C=C1CC[C@H](N(CCF)S(=O)(=O)c1ccc(F)cc1)C2]

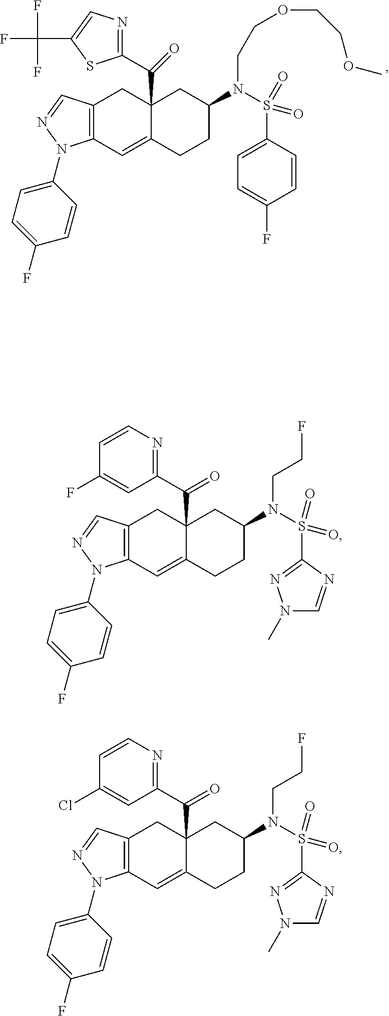 [SMILES: COCCOCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1 ; Cn1cnc(S(=O)(=O)N(CCF)[C@H]2CCC3=Cc4c(cnn4-c4ccc(F)cc4)C[C@]3(C(=O)c3cc(F)ccn3)C2)n1 ; Cn1cnc(S(=O)(=O)N(CCF)[C@H]2CCC3=Cc4c(cnn4-c4ccc(F)cc4)C[C@]3(C(=O)c3cc(Cl)ccn3)C2)n1]

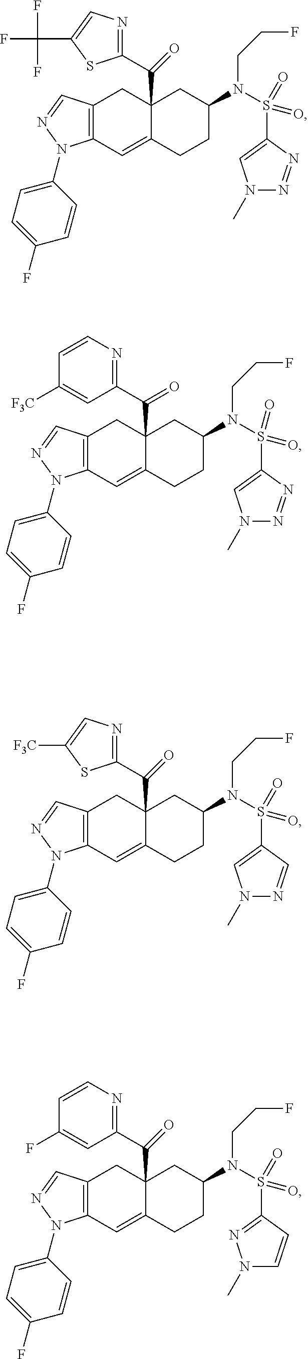 [SMILES: Cn1cc(S(=O)(=O)N(CCF)[C@H]2CCC3=Cc4c(cnn4-c4ccc(F)cc4)C[C@]3(C(=O)c3ncc(C(F)(F)F)s3)C2)nn1 ; Cn1cc(S(=O)(=O)N(CCF)[C@H]2CCC3=Cc4c(cnn4-c4ccc(F)cc4)C[C@]3(C(=O)c3cc(C(F)(F)F)ccn3)C2)nn1 ; Cn1cc(S(=O)(=O)N(CCF)[C@H]2CCC3=Cc4c(cnn4-c4ccc(F)cc4)C[C@]3(C(=O)c3ncc(C(F)(F)F)s3)C2)cn1 ; Cn1ccc(S(=O)(=O)N(CCF)[C@H]2CCC3=Cc4c(cnn4-c4ccc(F)cc4)C[C@]3(C(=O)c3cc(F)ccn3)C2)n1]

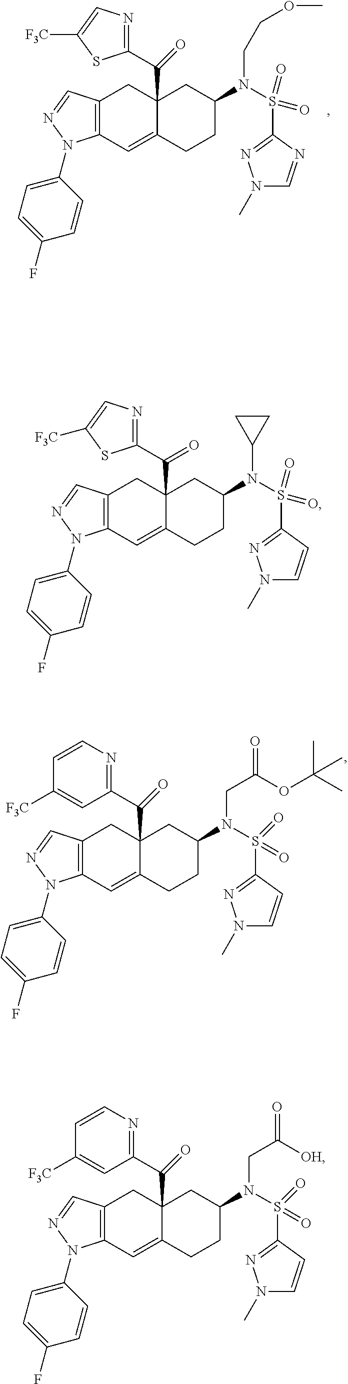 [SMILES: COCCN([C@H]1CCC2=Cc3c(cnn3-c3ccc(F)cc3)C[C@]2(C(=O)c2ncc(C(F)(F)F)s2)C1)S(=O)(=O)c1ncn(C)n1 ; Cn1ccc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N(CC(=O)OC(C)(C)C)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CC(=O)O)[C@H]2CCC3=Cc4c(cnn4-c4ccc(F)cc4)C[C@]3(C(=O)c3cc(C(F)(F)F)ccn3)C2)n1]

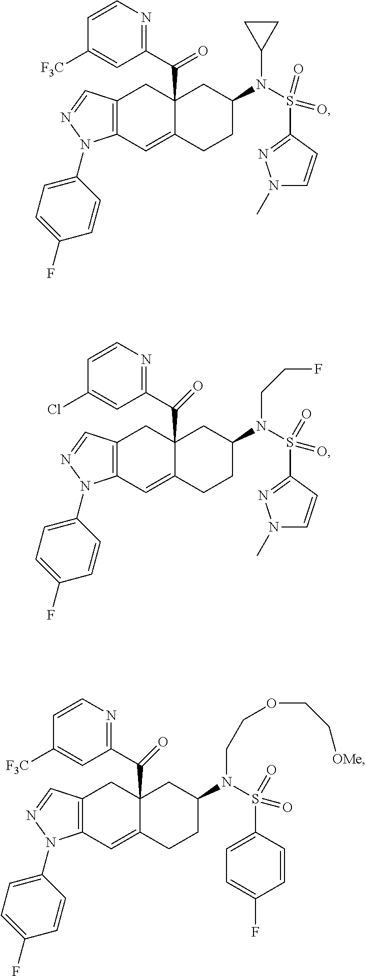 [SMILES: Cn1ccc(S(=O)(=O)N(C2CC2)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CCF)[C@H]2CCC3=Cc4c(cnn4-c4ccc(F)cc4)C[C@]3(C(=O)c3cc(Cl)ccn3)C2)n1 ; COCCOCCN([C@H]1CCC2=Cc3c(cnn3-c3ccc(F)cc3)C[C@]2(C(=O)c2cc(C(F)(F)F)ccn2)C1)S(=O)(=O)c1ccc(F)cc1]

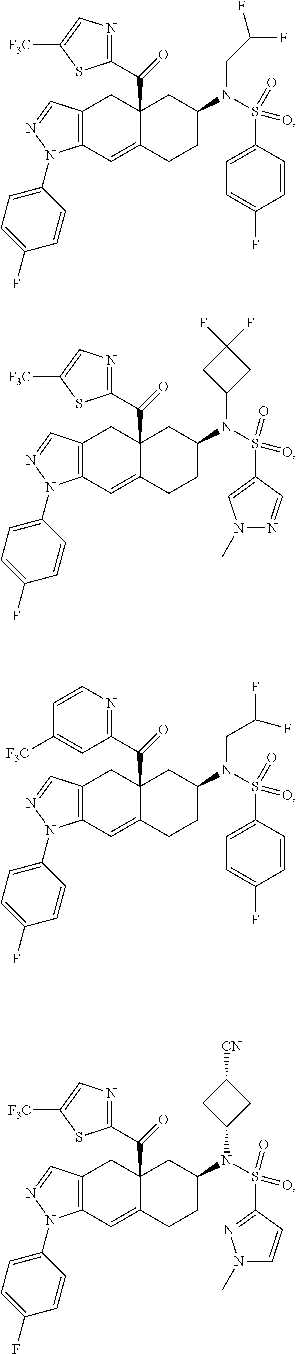 [SMILES: O=C(c1ncc(C(F)(F)F)s1)[C@]12Cc3cnn(-c4ccc(F)cc4)c3C=C1CC[C@H](N(CC(F)F)S(=O)(=O)c1ccc(F)cc1)C2 ; Cn1cc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)cn1 ; O=C(c1cc(C(F)(F)F)ccn1)[C@]12Cc3cnn(-c4ccc(F)cc4)c3C=C1CC[C@H](N(CC(F)F)S(=O)(=O)c1ccc(F)cc1)C2 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@H](C#N)C2)n1]

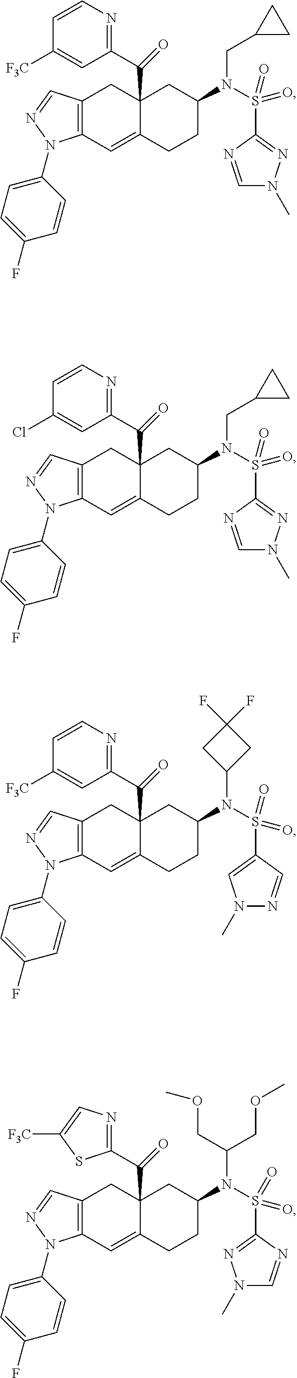

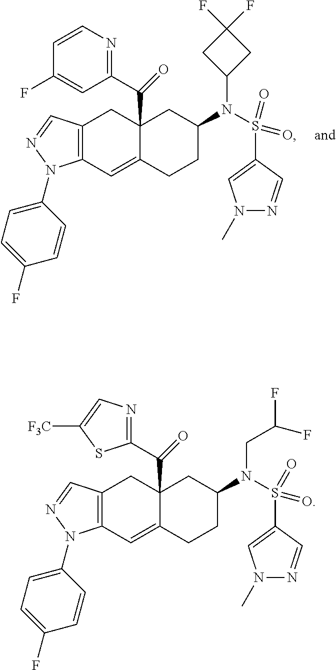 [SMILES: Cn1cc(S(=O)(=O)N(C2CC(F)(F)C2)[C@H]2CCC3=Cc4c(cnn4-c4ccc(F)cc4)C[C@]3(C(=O)c3cc(F)ccn3)C2)cn1 ; Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)cn1]

In some embodiments is a compound, or a pharmaceutically acceptable salt, solvate, or stereoisomer thereof, having a structure selected from:

[chemical structure]

[chemical structure]

[chemical structure]

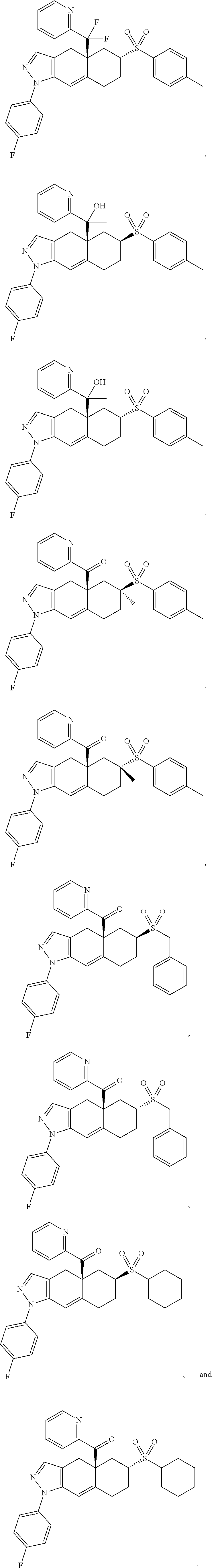

In some embodiments is a compound, or a pharmaceutically acceptable salt, solvate, or stereoisomer thereof, having a structure selected from:

[chemical structure]

[chemical structure]

[chemical structure]

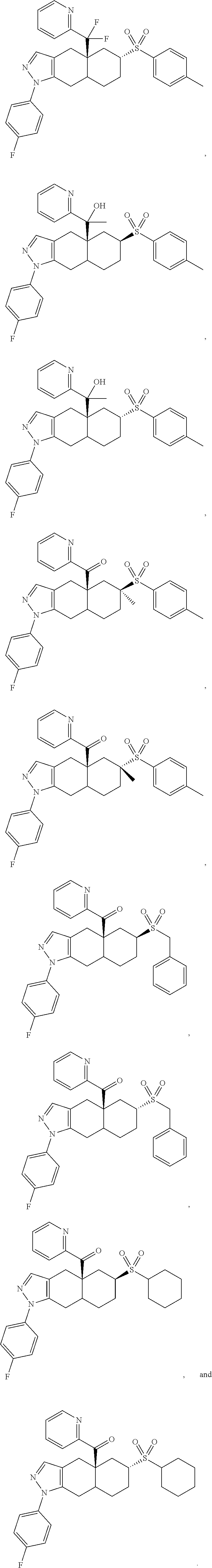

In some embodiments is a compound, or a pharmaceutically acceptable salt, solvate, or stereoisomer thereof, having a structure selected from:

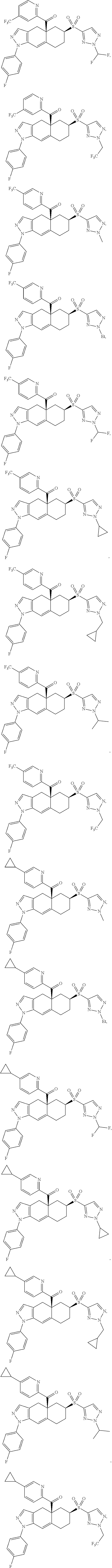

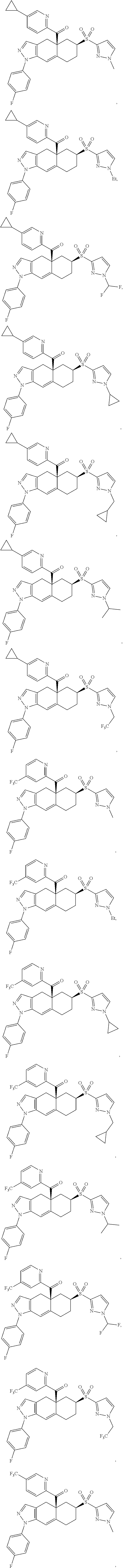

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

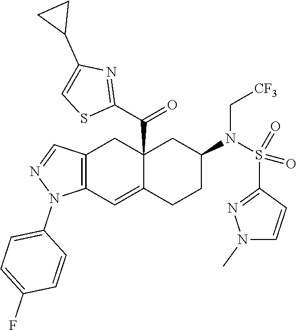 [SMILES: Cn1ccc(S(=O)(=O)N(CC(F)(F)F)[C@H]2CCC3=Cc4c(cnn4-c4ccc(F)cc4)C[C@]3(C(=O)c3nc(C4CC4)cs3)C2)n1]

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, the compounds described herein are prepared as outlined in Schemes 1-9.

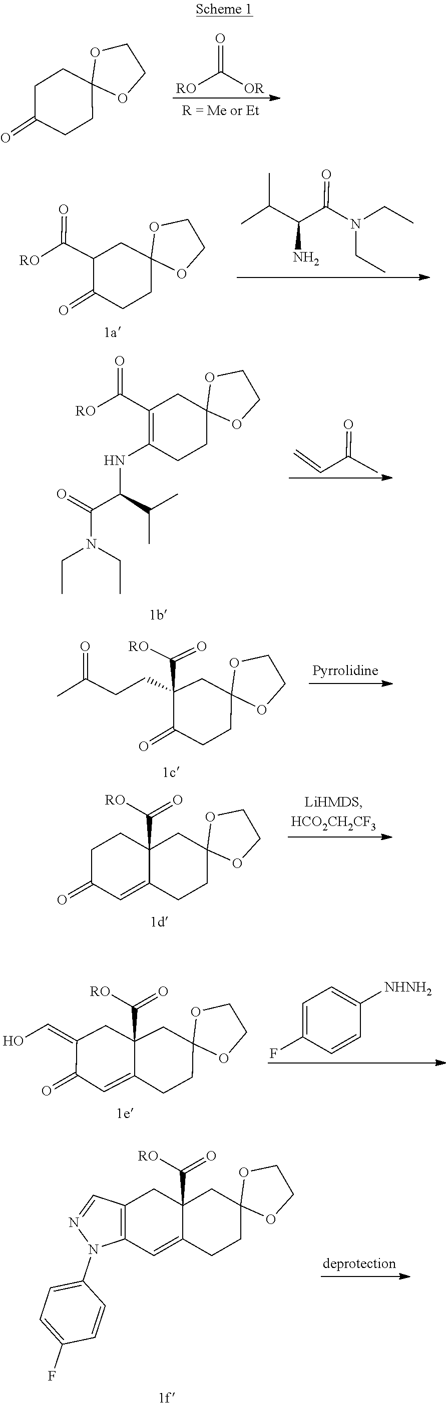

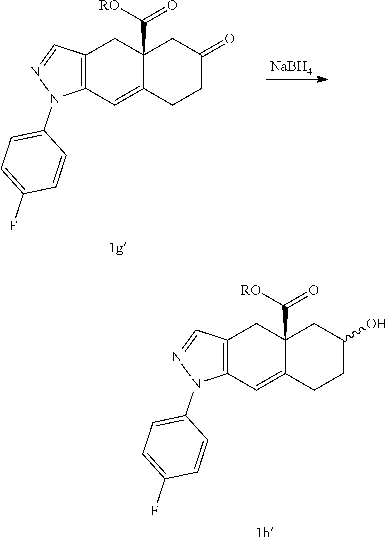

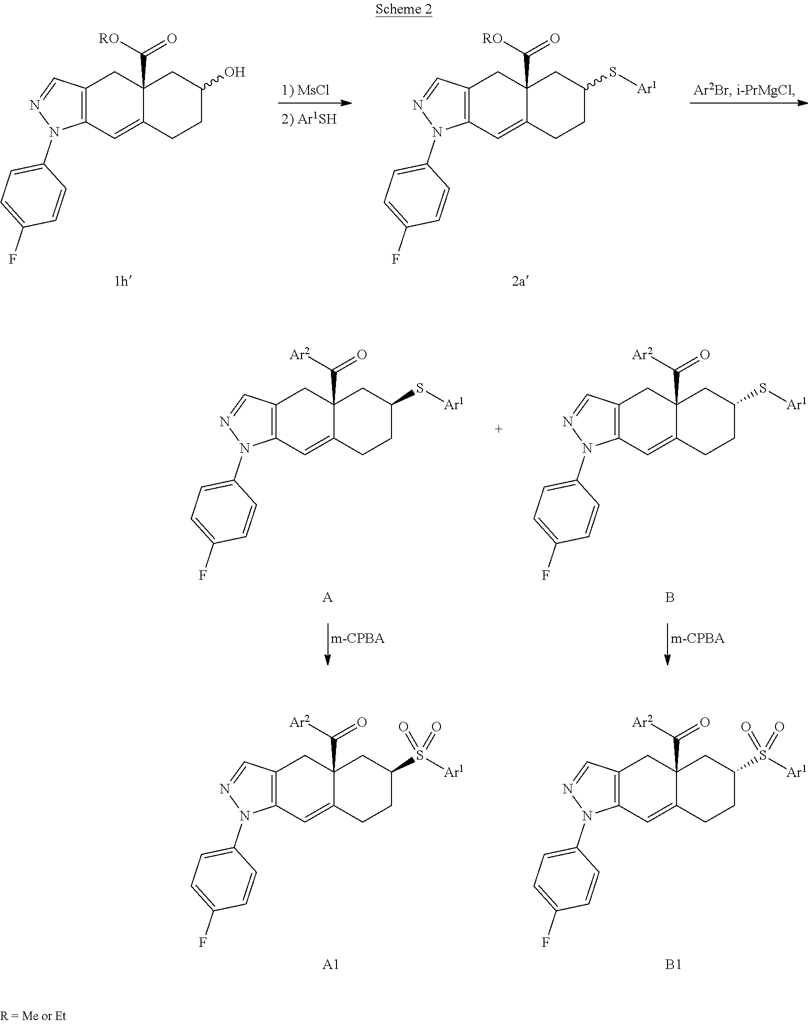

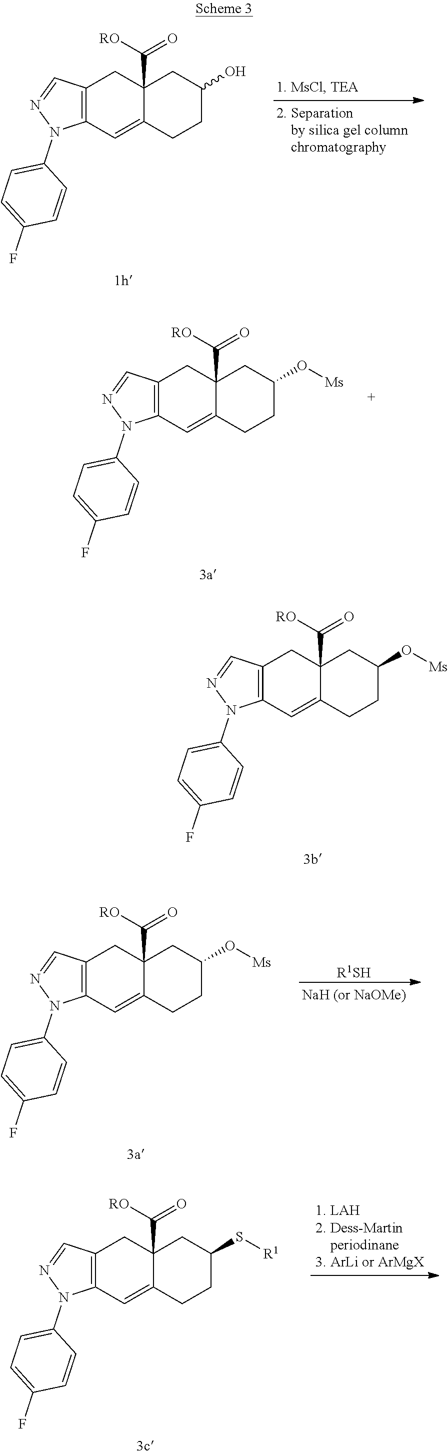

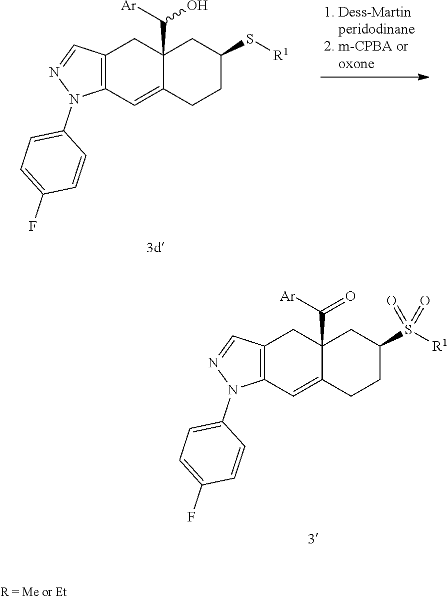

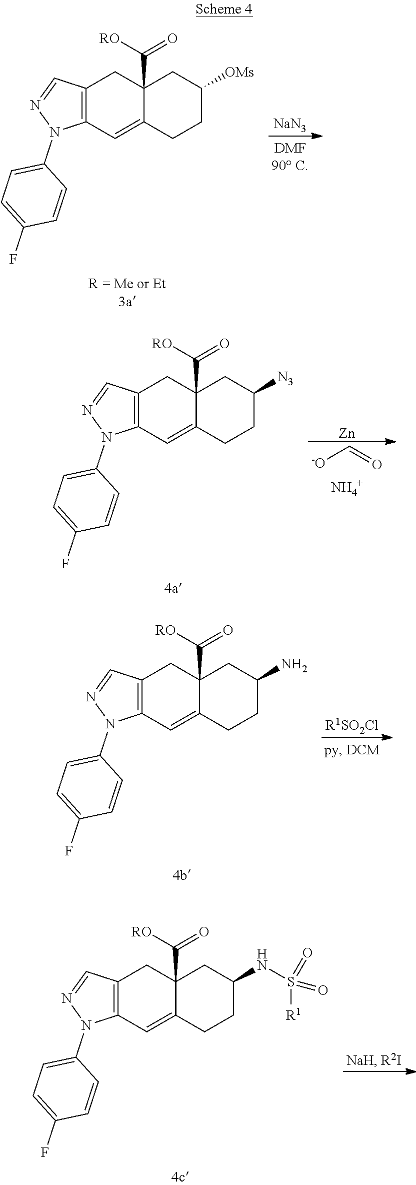

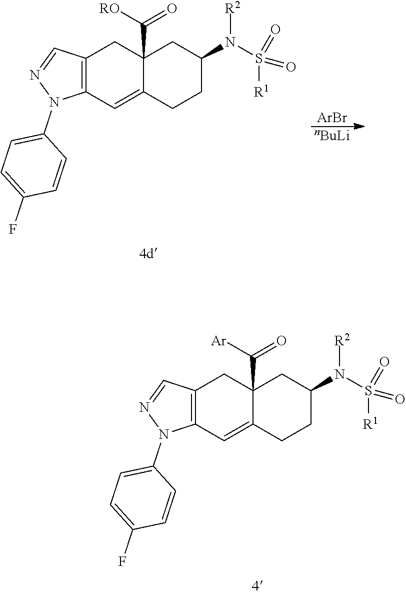

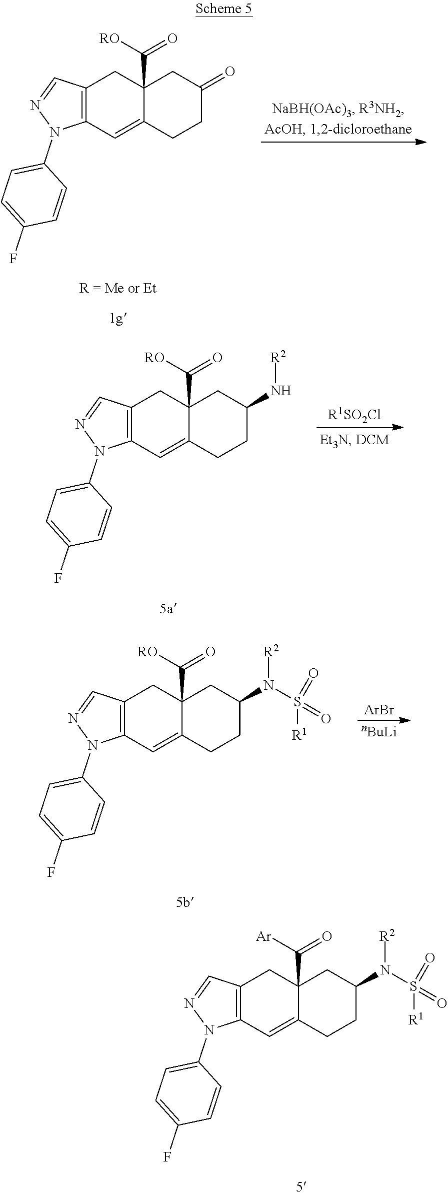

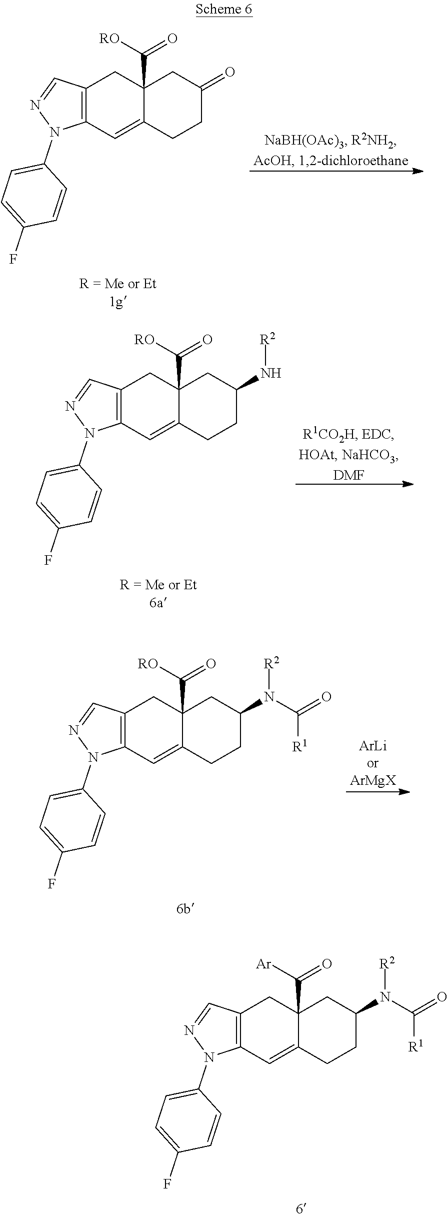

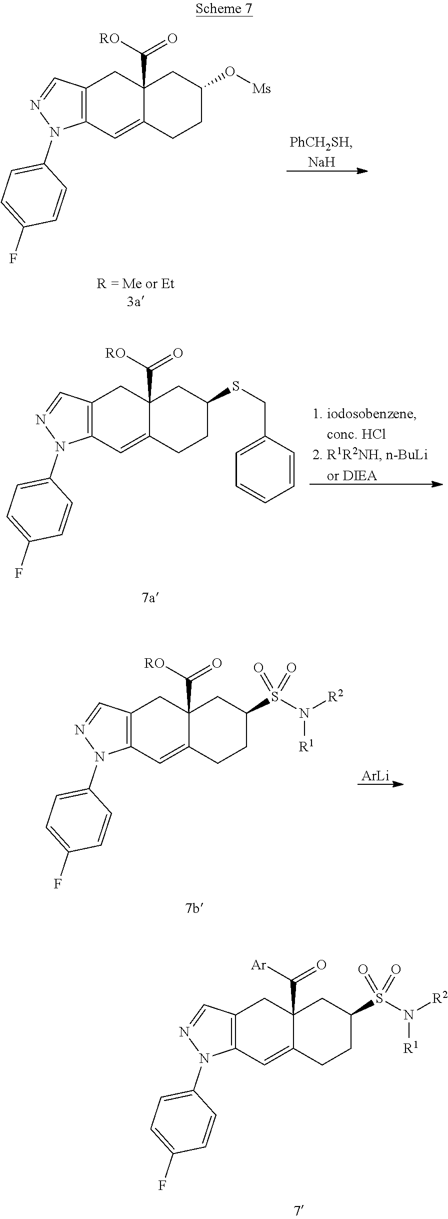

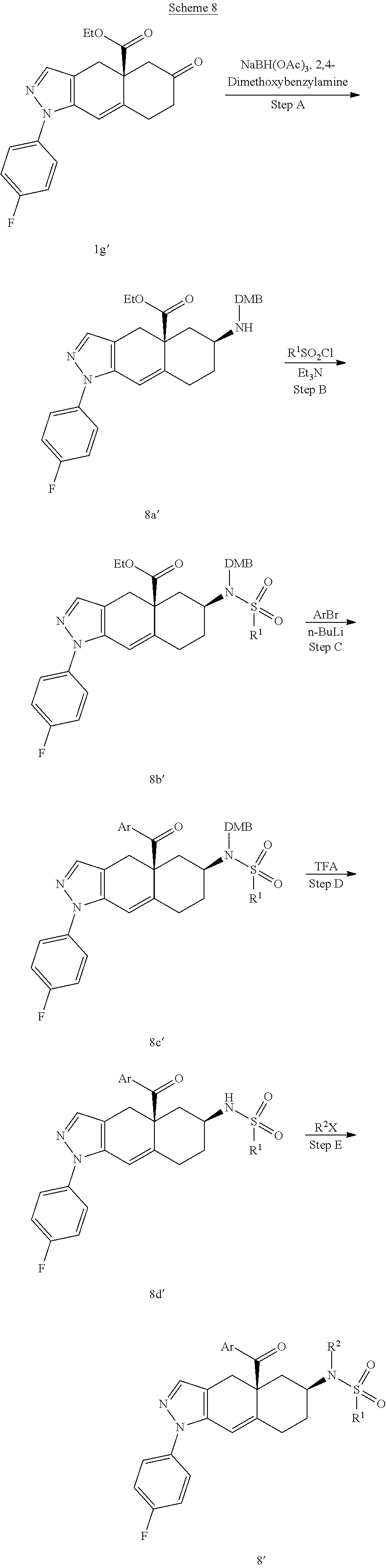

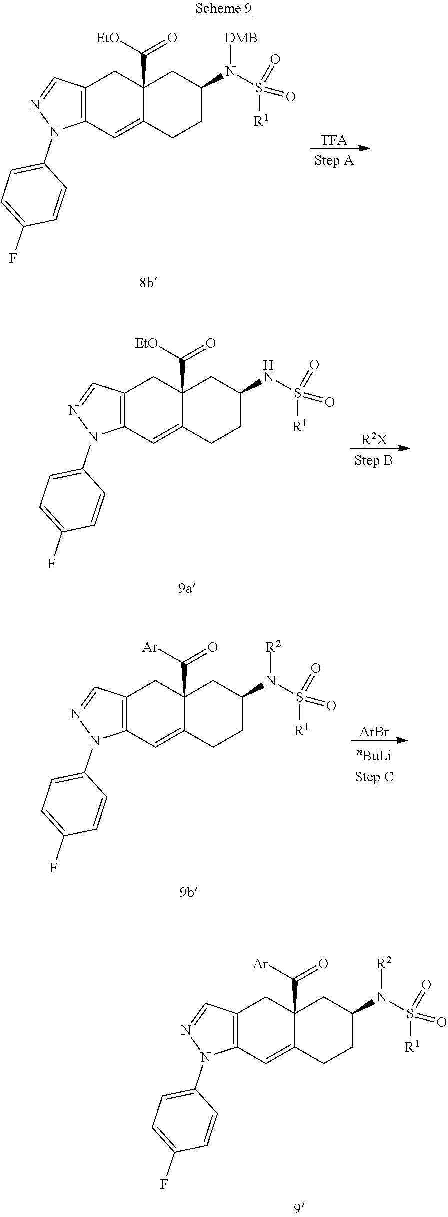

### Pharmaceutical Compositions

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising at least one compound described herein, or a pharmaceutically acceptable salt, solvate, or stereoisomer thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable excipient and a compound of Formula (I), or a pharmaceutically acceptable salt, solvate, or stereoisomer thereof.

One embodiment provides a pharmaceutical composition comprising a pharmaceutically acceptable excipient and a compound of Formula (Ia), or a pharmaceutically acceptable salt, solvate, or stereoisomer thereof.

One embodiment provides a pharmaceutical composition comprising a pharmaceutically acceptable excipient and a compound of Formula (Ib), or a pharmaceutically acceptable salt, solvate, or stereoisomer thereof.

One embodiment provides a pharmaceutical composition comprising a pharmaceutically acceptable excipient and a compound of Formula (Ic), or a pharmaceutically acceptable salt, solvate, or stereoisomer thereof.

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, Pa. (2005)).

The dose of the composition comprising at least one compound as described herein differ, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Use of the Compounds

### Glucocorticoid Receptor Modulators

Mifepristone is a non-selective modulator of several nuclear receptors. Mifepristone has been referred to as a GR antagonist, a progesterone receptor (PR) antagonist, a GR partial agonist, an androgen receptor (AR) antagonist and an AR partial agonist in the scientific literature. The activity observed at multiple hormone receptors leads to various undesirable side effects and in some instances, the promotion of cancer. Thus, AR agonism is an undesirable feature for GR antagonists used in the treatment of cancer (e.g., AR positive or AR dependent cancers including “castration resistant” prostate cancer (CRPC), breast cancer, or ovarian cancer). Antagonists of GR that have minimized binding to other hormone receptors, such as the androgen receptor (AR), are needed to effectively treat the diseases described herein with reduced side effects.

Some embodiments provided herein describe compounds disclosed herein that are modulators of glucocorticoid receptors (GR). In some embodiments, the compounds disclosed herein alter the level and/or activity of GR. In some embodiments, the compounds disclosed herein are GR antagonists. In some instances, glucocorticoid receptor antagonists bind to the receptor and prevent glucocorticoid receptor agonists from binding and eliciting GR mediated events, including regulation of transcription. Thus, in some embodiments, the compounds disclosed herein inhibit GR transcriptional activation activity. In some embodiments, the compounds disclosed herein are selective GR antagonists. In some embodiments, the compounds disclosed herein are not GR agonists. In some embodiments, the compounds disclosed herein are not GR partial agonists. In some embodiments, the compounds disclosed herein lessen cortisol activity in cells and make secondary therapeutic agents more effective.

In some embodiments, the compounds disclosed herein are useful for treating or preventing weight gain (e.g., Olanzapine induced weight gain), uterine fibrosis, alcoholism, alcohol abuse disorders, cocaine dependence, bipolar depression, adrenal hypercortisolism, post-traumatic stress disorder, anxiety disorders, mood disorders, hyperglycemia, and to induce abortion.

In some embodiments, the compounds disclosed herein are not androgen receptor (AR) signaling inhibitors. In these instances, the compounds disclosed herein do not significantly regulate AR levels and/or activity. In some embodiments, the compounds disclosed herein are not AR agonists. In some embodiments, the compounds disclosed herein have minimized binding to the androgen receptor (AR). In some embodiments, the compounds disclosed herein are not partial AR agonists. In some embodiments, the compounds disclosed herein have minimized partial AR agonism compared to mifepristone.

In some embodiments, the compounds disclosed herein are not partial AR agonists or partial GR agonists.

In some embodiments, the compounds disclosed herein do not modulate progesterone receptors. In some embodiments, the compounds described herein are not progesterone receptor (PR) inhibitors. In these instances, the compounds disclosed herein do not significantly regulate PR levels and/or activity. In some embodiments, the compounds disclosed herein are not PR agonists. In some embodiments, compounds disclosed herein are not PR partial agonists. In some embodiments, the compounds disclosed herein are not PR antagonists.

In some embodiments, the compounds disclosed herein are selective inhibitors. In some embodiments, use of the compounds disclosed herein in a patient does not cause or result in vaginal bleeding, cramping, nausea, vomiting, diarrhea, dizziness, back pain, weakness, tiredness, or combinations thereof. In certain embodiments, use of the compounds disclosed herein in a patient does not cause or result in vaginal bleeding. In certain embodiments, use of the compounds disclosed herein in a patient does not cause or result in cramping. In some embodiments, use of compounds disclosed herein in a patient does not cause or result in allergic reactions, low blood pressure, loss of consciousness, shortness of breath, rapid heartbeat, or combinations thereof.

### CYP Inhibition

CYPs are the major enzymes involved in drug metabolism, accounting for about 75% of the total metabolism. Most drugs undergo deactivation by CYPs, either directly or by facilitated excretion from the body. Also, many substances are bioactivated by CYPs to form their active compounds. In some instances, drugs increase or decrease the activity of various CYP isozymes either by inducing the biosynthesis of an isozyme (enzyme induction) or by directly inhibiting the activity of the CYP (enzyme inhibition). This activity is a major source of adverse drug interactions, since changes in CYP enzyme activity may affect the metabolism and clearance of various drugs. For example, if one drug inhibits the CYP-mediated metabolism of another drug, the second drug may accumulate within the body to toxic levels. Hence, in some instances, drug interactions necessitate dosage adjustments or choosing drugs that do not interact with the CYP system. Such drug interactions are especially important to take into account when using drugs of vital importance to the patient, drugs with adverse side-effects and drugs with small therapeutic windows, but any drug may be subject to an altered plasma concentration due to altered drug metabolism.

Cytochrome P₄₅₀2C₈ (abbreviated CYP2C8), a member of the cytochrome P450 mixed-function oxidase system, is involved in the metabolism of xenobiotics in the body. CYP2C8 is involved in the metabolism and clearance of various cancer drugs such as, for example, enzalutamide, paclitaxel, and sorafenib. In order to avoid drug-to-drug interaction caused by inhibition of the CYP2C8 isoform, a low level of CYP2C8 inhibition is desired.

Some embodiments provided herein describe GR antagonists that do not have clinically significant drug interactions resulting from inhibition or induction of CYP enzymes. In some embodiments, the GR antagonists do not have clinically significant drug interactions resulting from inhibition or induction of CYP2C8. In some embodiments, the compounds disclosed herein have reduced CYP inhibition. In some embodiments, the compounds disclosed herein have reduced CYP2C8 inhibition. In some embodiments, the compounds disclosed herein have <25% inhibition against CYP2C8 when paclitaxel is used as a substrate. In some embodiments, the compounds disclosed herein have <50% inhibition against CYP2C8 when paclitaxel is used as a substrate. In some embodiments, the compounds disclosed herein have <60% inhibition against CYP2C8 when paclitaxel is used as a substrate. In some embodiments, the compounds disclosed herein have <70% inhibition against CYP2C8 when paclitaxel is used as a substrate. In some embodiments, the compounds disclosed herein have <90% inhibition against CYP2C8 when paclitaxel is used as a substrate. In some embodiments, the compounds disclosed herein do not inhibit CYP2C8.

### Methods of Treatment

### Cancer

One embodiment provides a method of treating cancer in a subject in need thereof, comprising administering to the subject a compound disclosed herein provided herein, or a pharmaceutically acceptable salt thereof. In some embodiments, a compound disclosed herein is used in combination with a second therapeutic agent (e.g., an anti-cancer agent) for treating cancer. In some embodiments, the combination of the compound disclosed herein with the second therapeutic agent (e.g., an anti-cancer agent) provides a more effective initial therapy for treating cancer compared to the second therapeutic agent (e.g., an anti-cancer agent) administered alone. In some embodiments, a compound disclosed herein is used in combination with one or more additional therapeutic agents (e.g., anti-cancer agents) for treating cancer. In some embodiments, the combination of the compound disclosed herein with the one or more additional therapeutic agents (e.g., an anti-cancer agents) provides a more effective initial therapy for treating cancer compared to the one or more therapeutic agents (e.g., an anti-cancer agents) administered alone.

In some embodiments, the cancer is chemoresistant cancer, radio resistant cancer, anti-hormonal therapy resistant cancer, or treatment refractory cancer. In some embodiments, the cancer is relapsed cancer, persistent cancer, or recurrent cancer. Another embodiment provided herein describes a method of reducing incidences of cancer recurrence. Also provided here in some embodiments, is a method for treating a therapy-resistant cancer.

### Prostate Cancer

Prostate cancer is the second most common cause of cancer death in men in the United States, and approximately one in every six American men will be diagnosed with the disease during his lifetime. Treatment aimed at eradicating the tumor is unsuccessful in 30% of men.

One embodiment provides a method of treating prostate cancer in a subject in need thereof, comprising administering to the subject a compound disclosed herein provided herein, or a pharmaceutically acceptable salt thereof. In some embodiments, a compound disclosed herein is used in combination with a second therapeutic agent (e.g., an anti-cancer agent) for treating prostate cancer. In some embodiments, the combination of the compound disclosed herein with the second therapeutic agent (e.g., an anti-cancer agent) provides a more effective initial therapy for treating prostate cancer compared to the second therapeutic agent (e.g., an anti-cancer agent) administered alone. In some embodiments, a compound disclosed herein is used in combination with one or more additional therapeutic agents (e.g., anti-cancer agents) for treating prostate cancer. In some embodiments, the combination of the compound disclosed herein with the one or more additional therapeutic agents (e.g., an anti-cancer agents) provides a more effective initial therapy for treating prostate cancer compared to the one or more therapeutic agents (e.g., an anti-cancer agents) administered alone.

In some embodiments, the prostate cancer is chemoresistant cancer, radio resistant cancer, antiandrogen resistant, or refractory cancer. In some embodiments, the prostate cancer is relapsed cancer, persistent cancer, or recurrent cancer.

In some embodiments, the prostate cancer is acinar adenocarcinoma, atrophic carcinoma, foamy carcinoma, colloid carcinoma, or signet ring carcinoma. In some embodiments, the prostate cancer is ductal adenocarcinoma, transitional cell cancer, urothelial cancer, squamous cell cancer, carcinoid cancer, small cell cancer, sarcoma cancer, or sarcomatoid cancer. In some embodiments, the prostate cancer is metastatic castration-resistant prostate cancer, doubly-resistant prostate cancer, castration-resistant prostate cancer, hormone-resistant prostate cancer, androgen-independent, or androgen-refractory cancer.

In some instances, antiandrogens are useful for the treatment of prostate cancer during its early stages. In some instances, prostate cancer cells depend on androgen receptor (AR) for their proliferation and survival. Some prostate cancer patients are physically castrated or chemically castrated by treatment with agents that block production of testosterone (e.g. GnRH agonists), alone or in combination with antiandrogens, which antagonize effects of any residual testosterone.

In some instances, prostate cancer advances to a hormone-refractory state in which the disease progresses despite continued androgen ablation or antiandrogen therapy. The hormone-refractory state to which most patients eventually progress in the presence of continued androgen ablation or anti-androgen therapy is known as “castration resistant” prostate cancer (CRPC). CRPC is associated with an overexpression of AR. AR is expressed in most prostate cancer cells and overexpression of AR is necessary and sufficient for androgen-independent growth of prostate cancer cells. Failure in hormonal therapy, resulting from development of androgen-independent growth, is an obstacle for successful management of advanced prostate cancer.

While a small minority of CRPC does bypass the requirement for AR signaling, the vast majority of CRPC, though frequently termed “androgen independent prostate cancer” or “hormone refractory prostate cancer,” retains its lineage dependence on AR signaling.

Recently approved therapies that target androgen receptor (AR) signaling such as abiraterone acetate and enzalutamide have been utilized for treating CRPC. Despite these successes, sustained response with these agents is limited by acquired resistance which typically develops within 6-12 months. Doubly resistant prostate cancer is characterized in that tumor cells have become castration resistant and resistant when treated with second generation antiandrogens. Doubly resistant prostate cancer cells are characterized by a lack of effectiveness of second generation antiandrogens in inhibiting tumor growth.

In some embodiments, resistant prostate cancer (e.g., doubly resistant and castration resistant prostate cancers) occurs when cancer cells overexpress androgen receptors (AR). In some instances, increased signaling through the glucocorticoid receptor (GR) compensates for inhibition of androgen receptor signaling in resistant prostate cancer. Double resistant prostate cancer develops when expression of a subset of AR target genes is restored through activity of GR. In some instances, GR activation is responsible for this target gene activation. In some embodiments, GR transcription is activated in patients susceptible to or suffering from resistant prostate cancer (e.g., doubly resistant and castration resistant prostate cancers). In some instances, GR upregulation in cancer cells confers resistance to antiandrogens.

Some embodiments provided herein describe the use of a compound disclosed herein for treating prostate cancer in a subject in need thereof, including doubly resistant prostate cancer and castration resistant prostate cancer. In some embodiments, the subject in need has elevated tumor GR expression. In some embodiments, the compound disclosed herein is also an AR signaling inhibitor or antiandrogen.

In some embodiments, the compound disclosed herein is used in combination with a second therapeutic agent. In some embodiments, the compound disclosed herein is used in combination with one or more additional therapeutic agents. In some embodiments, the second or additional agent is an anti-cancer agent. In certain embodiments, the anti-cancer agent is useful for AR positive or AR negative prostate cancer.

### Breast Cancer

Breast cancer is the second leading cause of cancer among women in the United States. Triple-negative breast cancers are among the most aggressive and difficult to treat of all the breast cancer types. Triple-negative breast cancer is a form of the disease in which the three receptors that fuel most breast cancer growth—estrogen, progesterone and the HER-2—are not present. Because the tumor cells lack these receptors, treatments that target estrogen, progesterone and HER-2 are ineffective. Approximately 40,000 women are diagnosed with triple-negative breast cancer each year. It is estimated that more than half of these women's tumor cells express significant amounts of GR.

In some instances, GR expression is associated with a poor prognosis in estrogen receptor (ER)-negative early stage breast cancer. In some instances, GR activation in triple-negative breast cancer cells initiates an anti-apoptotic gene expression profile that is associated with inhibiting chemotherapy-induced tumor cell death. GR activity in these cancer cells correlates with chemotherapy resistance and increased recurrence of cancer.

Provided herein in some embodiments are methods of treating breast cancer, the method comprising administering to a subject in need thereof a compound disclosed herein provided herein, or a pharmaceutically acceptable salt thereof. In some embodiments, a compound disclosed herein is used in combination with a second therapeutic agent (e.g., a chemotherapeutic agent) for treating breast cancer. In some embodiments, the combination of the compound disclosed herein with the second therapeutic agent (e.g., a chemotherapeutic agent) provides a more effective initial therapy for treating breast cancer compared to the second therapeutic agent (e.g., a chemotherapeutic agent) administered alone. In some embodiments, a compound disclosed herein is used in combination with one or more additional therapeutic agents (e.g., anti-cancer agents) for treating breast cancer. In some embodiments, the combination of the compound disclosed herein with the one or more additional therapeutic agents (e.g., an anti-cancer agents) provides a more effective initial therapy for treating breast cancer compared to the one or more therapeutic agents (e.g., an anti-cancer agents) administered alone.

In some embodiments, the breast cancer is chemoresistant cancer, radio resistant cancer, antihormonal therapy resistant cancer, or refractory cancer. In some embodiments, the breast cancer is relapsed cancer, persistent cancer, or recurrent cancer. Breast cancers may include, but are not limited to, ductal carcinoma, invasive ductal carcinoma, tubular carcinoma of the breast, medullary carcinoma of the breast, mecinous carcinoma of the breast, papillary carcinoma of the breast, cribriform carcinoma of the breast, invasive lobular carcinoma, inflammatory breast cancer, lobular carcinoma in situ, male breast cancer, Paget disease of the nipple, phyllodes tumor of the breast, recurrent and metastatic breast cancer, triple-negative breast cancer, or combinations thereof.

In some embodiments, the breast cancer is recurrent and metastatic breast cancer, triple-negative breast cancer, or combinations thereof. In some embodiments, the breast cancer is chemoresistant triple-negative breast cancer or estrogen receptor (ER) negative breast cancer. In some embodiments, the breast cancer is chemoresistant triple-negative breast cancer. In some embodiments, the breast cancer is estrogen receptor (ER) negative breast cancer. In some embodiments, the breast cancer is GR+ triple-negative breast cancer. In some embodiments, the breast cancer is GR+ estrogen receptor (ER) negative breast cancer.

Some embodiments provided herein describe the use of a compound disclosed herein for treating breast cancer in a patient, including triple negative breast cancer or ER negative breast cancer. In some embodiments, the compound described herein inhibits the anti-apoptotic signaling pathways of GR and increase the cytotoxic efficiency of secondary chemotherapeutic agents. In some embodiments, the compounds described herein enhance the efficacy of chemotherapy in breast cancer patients, such as triple negative breast cancer patients. In some embodiments, the breast cancer patient has elevated tumor GR expression.

Some embodiments provided herein describe methods of treating estrogen positive breast cancer. In some instances, estrogen positive breast cancer patients become resistant to estrogen receptor modulators. In some embodiments, the compound disclosed herein enhances the efficacy of estrogen receptor modulators in estrogen positive breast cancer patients. In some embodiments, the breast cancer patient has elevated tumor GR expression. In some embodiments, a GR inhibitor described herein is used in combination with an estrogen receptor modulator. In some embodiments, the estrogen receptor modulator is tamoxifen, raloxifene, toremifene, tibolone, fulvestrant, lasofoxifene, clomifene, ormeloxifene, or ospemifene. In some embodiments, the estrogen receptor modulator is tamoxifen, raloxifene, toremifene, tibolone, or fulvestrant. In some embodiments, the estrogen receptor modulator is tamoxifen, raloxifene, or toremifene. In certain embodiments, the estrogen receptor modulator is tamoxifen.

### Ovarian Cancer

Ovarian cancer is the leading cause of death from gynecologic malignancies. Some ovarian cancers (e.g., high grade serous ovarian cancer) are initially sensitive to platinum-based therapy, but relapse rates remain high.

One embodiment provides a method of treating ovarian cancer in a patient in need thereof, comprising administering to the patient a compound disclosed herein provided herein, or a pharmaceutically acceptable salt thereof. In some embodiments, the patient has elevated tumor GR expression. In some embodiments, a compound disclosed herein is used in combination with a second therapeutic agent (e.g., a chemotherapeutic agent) for treating ovarian cancer. In some embodiments, the combination of the compound disclosed herein with the second therapeutic agent (e.g., a chemotherapeutic agent) provides a more effective initial therapy for treating ovarian cancer compared to the second therapeutic agent (e.g., a chemotherapeutic agent) administered alone. In some embodiments, a compound disclosed herein is used in combination with one or more additional therapeutic agents (e.g., anti-cancer agents) for treating ovarian cancer. In some embodiments, the combination of the compound disclosed herein with the one or more additional therapeutic agents (e.g., an anti-cancer agents) provides a more effective initial therapy for treating ovarian cancer compared to the one or more therapeutic agents (e.g., an anti-cancer agents) administered alone.

In some instances, GR activation increases resistance to chemotherapy in ovarian cancer (e.g., high-grade serous ovarian cancer). In some instances, GR activation significantly inhibits chemotherapy induced apoptosis in ovarian cancer cells. Provided herein in some embodiments are methods of treating ovarian cancer in a subject, the method comprising treating the subject with a compound disclosed herein to improve sensitivity to chemotherapy. In some embodiments, the ovarian cancer has become resistant to chemotherapy. In some embodiments, the ovarian cancer cells are resistant to cisplatin, carboplatin, paclitaxel, docetaxel, nab-paclitaxel, cabazitaxel, gemcitabine, pemetrexed, alone or in combination. In some embodiments, the ovarian cancer cells are resistant to cisplatin, paclitaxel, carboplatin, gemcitabine, alone or in combination. In some embodiments, the compound disclosed herein reverses the cell survival effect.

Ovarian cancers may include, but are not limited to, epithelial ovarian cancers, such as serous epithelial ovarian cancer, endometrioid epithelial ovarian cancer, clear cell epithelial ovarian cancer, mucinous epithelial ovarian cancer, undifferentiated or unclassifiable epithelial ovarian cancer, refractory ovarian cancer, sex cord-stromal tumors, Sertoli and Sertoli-Leydig cell tumors, germ cell tumors, such as dysgerminoma and nondysgerminomatous tumors, Brenner tumors, primary peritoneal carcinoma, fallopian tube cancer, or combinations thereof.

### Non-Small Cell Lung Cancer

One embodiment provides a method of treating non-small cell lung cancer (NSCLC) in a patient in need thereof, comprising administering to the patient a compound disclosed herein provided herein, or a pharmaceutically acceptable salt thereof. In some embodiments, the patient has elevated tumor GR expression. In some embodiments, a compound disclosed herein is used in combination with a second therapeutic agent (e.g., a chemotherapeutic agent) for treating NSCLC. In some embodiments, the combination of the compound disclosed herein with the second therapeutic agent (e.g., a chemotherapeutic agent) provides a more effective initial therapy for treating NSCLC compared to the second therapeutic agent (e.g., a chemotherapeutic agent) administered alone. In some embodiments, a compound disclosed herein is used in combination with one or more additional therapeutic agents (e.g., anti-cancer agents) for treating NSCLC. In some embodiments, the combination of the compound disclosed herein with the one or more additional therapeutic agents (e.g., an anti-cancer agents) provides a more effective initial therapy for treating NSCLC compared to the one or more therapeutic agents (e.g., an anti-cancer agents) administered alone.

### Hypercortisolism/Cushing's Disease

One embodiment provides a method of treating hypercortisolism or Cushing's disease in a patient in need thereof, comprising administering to the patient a compound disclosed herein provided herein, or a pharmaceutically acceptable salt thereof.

Types of Cushing's disease include, but are not limited to, recurrent Cushing's disease, refractory Cushing's disease, persistent Cushing's disease, endogenous Cushing's disease, spontaneous hypercortisolism, Adrenocorticotropic hormone dependent, Adrenocorticotropic hormone independent, or combinations thereof.

Causes of hypercortisolism may include, but are not limited to, prolonged exposure to cortisol, a tumor that produces excessive cortisol, a tumor that results in the excess production of cortisol, or combinations thereof.

### Combination Treatment

In some embodiments, a compound disclosed herein is used in combination with at least a second therapeutic agent, such as a chemotherapeutic agent or immunotherapy. In some embodiments, the compound disclosed herein is used in combination with one or more additional therapeutic agents. In some embodiments, the second or additional therapeutic agent is cisplatin, carboplatin, cyclophosphamide, capecitabine, gemcitabine, paclitaxel, nab-paclitaxel, altretamine, docetaxel, epirubicin, melphalan, methotrexate, mitoxantrone, ixabepilone, ifosfamide, irinotecan, eribulin, etoposide, doxorubicin, liposomal doxorubicin, camptothecin, pemetrexed, topotecan, vinorelbine, vinblastine, daunorubicin, fluorouracil, mitomycin, thiotepa, vincristine, everolimus, veliparib, glembatumumab vedotin, pertuzumab, trastuzumab, or any combinations or any salts thereof. In some embodiments, cisplatin, carboplatin, paclitaxel, docetaxel, nab-paclitaxel, cabazitaxel, gemcitabine, pemetrexed, or any combinations or any salts thereof. In some embodiments, the second or additional therapeutic agent is gemcitabine. In some embodiments, the second or additional therapeutic agent is carboplatin. In some embodiments, the second or additional therapeutic agent is cisplatin. In some embodiments, the second or additional agent is paclitaxel. In some embodiments, the compound disclosed herein is used in combination with gemcitabine and carboplatin. In some embodiments, the compound disclosed herein is used in combination with carboplatin and cisplatin. In some embodiments, the second or additional therapeutic agent is an anti-PD-L1 agent. In certain embodiments, the anti-PD-L1 agent is atezolizumab (MPDL3280A) or avelumab. In some embodiments, the second or additional therapeutic agent is an anti-PD1 agent. In certain embodiments, the anti-PD1 agent is nivolumab or pembrolizumab. In some embodiments, the second or additional therapeutic agent is an anti-CTLA-4 agent. In some embodiments, the second or additional therapeutic agent is a CAR-T cells therapy. In some embodiments, the second or additional therapeutic agent is a cancer vaccine. In some embodiments, the second or additional therapeutic agent is an IDO-1 inhibitor.

In some embodiments, the second or additional agent is an AR signaling inhibitor or antiandrogen. In certain embodiments, the AR signaling inhibitor is an AR antagonist. In some embodiments, the second or additional therapeutic agent is selected from finasteride, dutasteride, alfatradiol, cyproterone acetate, spironolactone, danazol, gestrinone, ketoconazole, abiraterone acetate, enzalutamide, apalutamide, danazol, gestrinone, danazol, simvastatin, aminoglutethimide, atorvastatin, simvastatin, progesterone, cyproterone acetate, medroxyprogesterone acetate, megestrol acetate, chlormadinone acetate, spironolactone, drospirenone, estradiol, ethinyl estradiol, diethylstilbestrol, conjugated equine estrogens, buserelin, deslorelin, gonadorelin, goserelin, histrelin, leuprorelin, nafarelin, triptorelin, abarelix, cetrorelix, degarelix, ganirelix, or any combinations or any salts thereof. In some embodiments, the second or additional therapeutic agent is selected from flutamide, nilutamide, bicalutamide, enzalutamide, apalutamide, cyproterone acetate, megestrol acetate, chlormadinone acetate, spironolactone, canrenone, drospirenone, ketoconazole, topilutamide, cimetidine, or any combinations or any salts thereof. In some embodiments, the AR signaling inhibitor is 3,3′-diindolylmethane (DIM), abiraterone acetate, apalutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof. In some embodiments, the AR signaling inhibitor is flutamide, nilutamide, bicalutamide, or megestrol. In other embodiments, the androgen receptor signaling inhibitor is enzalutamide and apalutamide. In some embodiments, the AR signaling inhibitor is apalutamide. In other embodiments, the AR signaling inhibitor is enzalutamide.

In some embodiments, the anti-cancer agent is mitoxantrone, estramustine, etoposide, vinblastine, carboplatin, vinorelbine, paclitaxel, daunomycin, darubicin, epirubicin, docetaxel, nab-paclitaxel, cabazitaxel, pemetrexed, or doxorubicin. In some embodiments, the anti-cancer agent is paclitaxel, daunomycin, darubicin, epirubicin, docetaxel, cabazitaxel, or doxorubicin. In certain embodiments, the anti-cancer agent is docetaxel.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

Unless otherwise noted, reagents and solvents were used as received from commercial suppliers.

Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: ((4aS,6S)-1-(4-Fluorophenyl)-6-tosyl-1,4,5,6,7,8-hexahydro-4aH-benzo[f]indazol-4a-yl)(pyridin-2-yl)methanone (1)

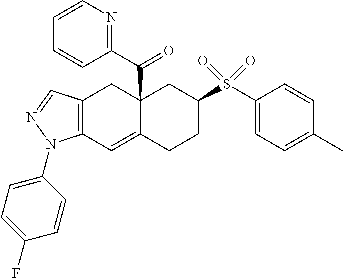 [SMILES: Cc1ccc(S(=O)(=O)[C@H]2CCC3=Cc4c(cnn4-c4ccc(F)cc4)C[C@]3(C(=O)c3ccccn3)C2)cc1]

### Step A: Ethyl 8-oxo-1,4-dioxaspiro[4.5]decane-7-carboxylate (1a)

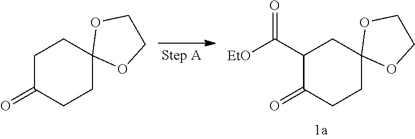 [SMILES: O=C1CCC2(CC1)OCCO2 ; CCOC(=O)C1CC2(CCC1=O)OCCO2]

A solution of diethyl carbonate (567.7 g, 4.81 mmol) and 1,4-dioxaspiro[4.5]decan-8-one (75 g, 0.481 mol) in anhydrous THF (300 mL) was added to a suspension of sodium hydride (60% in mineral oil, 48 g, 1.2 mol) in anhydrous THF (500 mL). After the mixture was refluxed with stirring for 3 h, it was cooled down to 0° C., neutralized with AcOH (pH 7), and diluted with water. The solution was extracted with EtOAc and the combined organic layers were washed with saturated NaHCO₃ solution, and brine, dried over Na₂SO₄, filtered and concentrated. The residue was purified by column chromatography on silica gel to afford the title compound (1a) (76.2 g, 70%) as a colorless oil. m/z (ESI, +ve ion)=250.96 [M+Na]⁺.

### Step B: Ethyl (S)-8-((1-(diethylamino)-3-methyl-1-oxobutan-2-yl)amino)-1,4-dioxaspiro[4.5]dec-7-ene-7-carboxylate (1b)

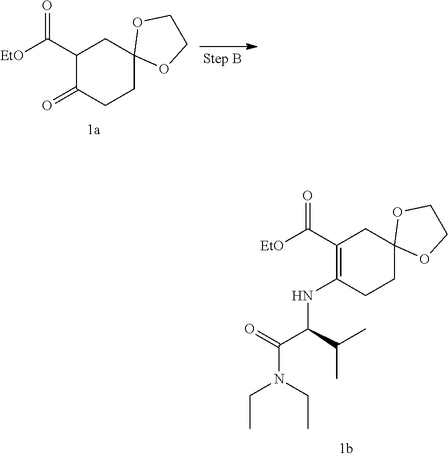 [SMILES: CCOC(=O)C1CC2(CCC1=O)OCCO2 ; CCOC(=O)C1=C(N[C@H](C(=O)N(CC)CC)C(C)C)CCC2(C1)OCCO2]

(S)-2-Amino-N,N-diethyl-3-methylbutanamide (33 g, 0.145 mol), molecular sieves (6.0 g, 4 Å), and concentrated hydrochloric acid (2 mL) were successively added to a solution of ethyl 8-oxo-1,4-dioxaspiro[4.5]decane-7-carboxylate (1a) (50 g, 0.29 mol) in toluene (400 mL). After the reaction mixture was stirred for 16 h at 50° C., it was filtrated, the residue was washed with DCM and filtrate was evaporated under vacuum. The crude product was purified by column chromatography on neutral aluminum oxide to provide the title compound (1b) (28.7 g, 51%).

### Step C: Ethyl (S)-8-oxo-7-(3-oxobutyl)-1,4-dioxaspiro[4.5]decane-7-carboxylate (1c)

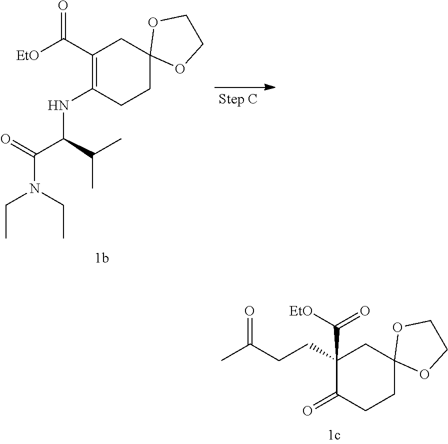 [SMILES: CCOC(=O)C1=C(N[C@H](C(=O)N(CC)CC)C(C)C)CCC2(C1)OCCO2 ; CCOC(=O)[C@@]1(CCC(C)=O)CC2(CCC1=O)OCCO2]

A mixture of ethyl (S)-8-((1-(diethylamino)-3-methyl-1-oxobutan-2-yl)amino)-1,4-dioxaspiro[4.5]dec-7-ene-7-carboxylate (1b) (25.0 g, 65.4 mmol), Cu(OAc)₂.H₂O (1.19 g, 6.54 mmol) in acetone (250 mL) was stirred for 30 min at rt, methyl vinyl ketone (13.93 g, 0.196 mmol) was added and the mixture was stirred at rt for 3 days. All volatile materials were removed under vacuum and the residue was diluted with 10% aqueous acetic acid. The resulting solution was stirred at rt overnight and extracted with DCM. The combined organic layers were washed with sat. aq NaHCO₃ and brine, dried, and concentrated under reduced pressure. The residue was purified by column chromatography on silica gel to provide the title compound (1c) (15.4 g, 67%).

### Step D: Ethyl (S)-6-oxo-4,6,7,8-tetrahydro-1H-spiro[naphthalene-2,2′-[1,3]dioxolane]-8a(3H)-carboxylate (1d)

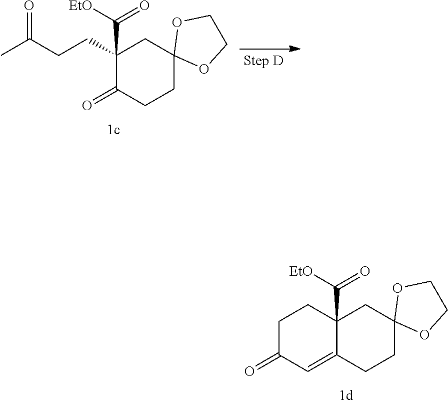 [SMILES: CCOC(=O)[C@@]1(CCC(C)=O)CC2(CCC1=O)OCCO2 ; CCOC(=O)[C@]12CCC(=O)C=C1CCC1(C2)OCCO1]

Pyrrolidine (0.734 g, 10.3 mmol) and AcOH (0.62 g, 10.3 mmol) were added to a solution of ethyl (S)-8-oxo-7-(3-oxobutyl)-1,4-dioxaspiro[4.5]decane-7-carboxylate (1c) (15.4 g, 51.7 mmol) in toluene (160 mL). After being stirred at 100° C. for 2 h, the reaction mixture was cooled down to rt and washed with sat. aq NaHCO₃, and brine, dried, and concentrated under reduced pressure. The residue was purified by column chromatography on silica gel to give the title compound (id) (12.2 g, 84%) as a yellow oil.

### Step E: Ethyl (S,Z)-7-(hydroxymethylene)-6-oxo-4,6,7,8-tetrahydro-1H-spiro[naphthalene-2,2′-[1,3]dioxolane]-8a(3H)-carboxylate (1e)

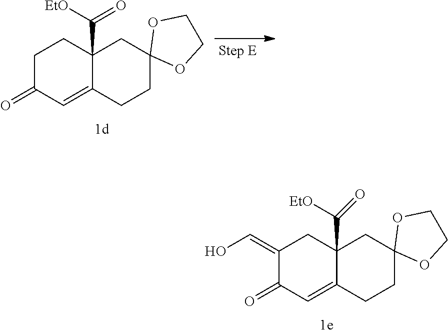 [SMILES: CCOC(=O)[C@]12CCC(=O)C=C1CCC1(C2)OCCO1 ; CCOC(=O)[C@]12C/C(=C/O)C(=O)C=C1CCC1(C2)OCCO1]

A solution of ethyl (S)-6-oxo-4,6,7,8-tetrahydro-1H-spiro[naphthalene-2,2′-[1,3]dioxolane]-8a(3H)-carboxylate (1d) (11.9 g, 42.5 mmol) in ether (80 mL) was added to lithium hexamethyldisilazide (255 mL, 255 mmol) in diethyl ether (200 mL) at −78° C. After 20 min, 2,2,2-trifluoroethyformate (54.4 g, 0.425 mol) was added. The reaction was stirred at −78° C. for 2 h and then allowed to slowly warm to rt. The reaction was quenched with sat. NH₄Cl and extracted with DCM. The organic phase was separated, washed with brine, dried and concentrated to give the title compound (1e) (13.0 g), which was used for the next step without further purification. m/z (ESI, +ve ion)=306.85 [M−H]⁻.

### Step F: Ethyl (S)-1-(4-fluorophenyl)-1,4,7,8-tetrahydrospiro[benzo[f]indazole-6,2′-[1,3]dioxolane]-4a(5H)-carboxylate (1f)

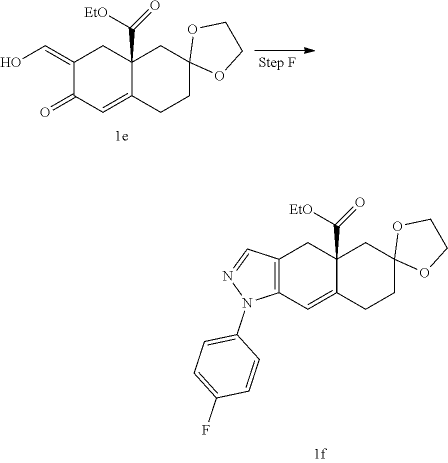 [SMILES: CCOC(=O)[C@]12C/C(=C/O)C(=O)C=C1CCC1(C2)OCCO1 ; CCOC(=O)[C@]12Cc3cnn(-c4ccc(F)cc4)c3C=C1CCC1(C2)OCCO1]

To a suspension of ethyl (S,Z)-7-(hydroxymethylene)-6-oxo-4,6,7,8-tetrahydro-1H-spiro[naphthalene-2,2′-[1,3]dioxolane]-8a(3H)-carboxylate (1e) (13.0 g, 42.2 mmol) in acetic acid (90 mL) were added sodium acetate (3.81 g, 46.4 mmol) and 4-fluorophenylhydrazine (7.2 g, 44.3 mmol). The reaction mixture was stirred at rt for 3 h and was diluted with water, extracted with EtOAc. The combined organic layers were washed with brine, dried, and concentrated under reduced pressure. The resulting oil was purified by column chromatography on silica gel to provide the title compound (If) (13 g, 91%) as a yellow solid. m/z (ESI, +ve ion)=398.65 [M+H]⁺.

### Step G: Ethyl (S)-1-(4-fluorophenyl)-6-oxo-1,4,5,6,7,8-hexahydro-4aH-benzo[f]indazole-4a-carboxylate (1 g)

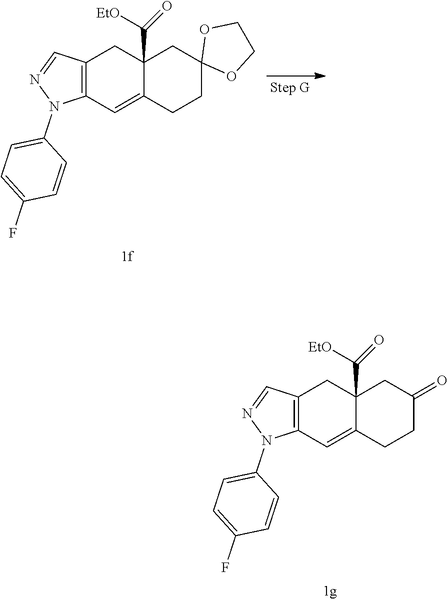 [SMILES: CCOC(=O)[C@]12Cc3cnn(-c4ccc(F)cc4)c3C=C1CCC1(C2)OCCO1 ; CCOC(=O)[C@@]12CC(=O)CCC1=Cc1c(cnn1-c1ccc(F)cc1)C2]

To a solution of ethyl (S)-1-(4-fluorophenyl)-1,4,7,8-tetrahydrospiro[benzo[f]indazole-6,2′-[1,3]dioxolane]-4a(5H)-carboxylate (f) (13 g, 32.7 mmol) in acetone (140 mL) was added 4 N aqueous HCl (140 mL). The reaction mixture was stirred at rt overnight and was diluted with EtOAc, basified with sat. aq. NaHCO₃, and extracted EtOAc. The combined organic layers were washed with brine, dried, and concentrated under reduced pressure to give the title compound (1g) which was used in the next step without further purification. m/z (ESI, +ve ion)=354.85 [M+H]⁺.

### Step H: Ethyl (4aS)-1-(4-fluorophenyl)-6-hydroxy-1,4,5,6,7,8-hexahydro-4aH-benzo[f]indazole-4a-carboxylate (1h)

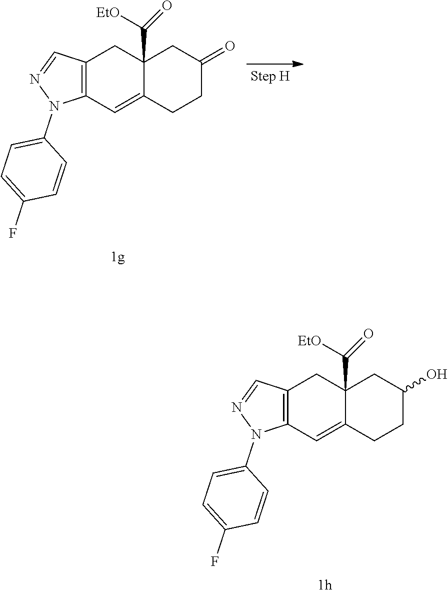 [SMILES: CCOC(=O)[C@@]12CC(=O)CCC1=Cc1c(cnn1-c1ccc(F)cc1)C2 ; CCOC(=O)[C@]12Cc3cnn(-c4ccc(F)cc4)c3C=C1CCC(O)C2]

To a solution of ethyl (S)-1-(4-fluorophenyl)-6-oxo-1,4,5,6,7,8-hexahydro-4aH-benzo[f]indazole-4a-carboxylate (1 g) (11.6 g, 32.8 mmol) in MeOH (450 mL) was added NaBH₄ (2.24 g, 59 mmol) at 0° C. The mixture was stirred at 0° C. for 2 h and then quenched with water and extracted with EtOAc. The combined organic layers were dried and concentrated under reduced pressure. The residue was purified by chromatography on silica gel to give the title compound (1h) (9.4 g, 74.3%) as a diastereometric mixture. m/z (ESI, +ve ion)=356.72 [M+H]⁺.

### Step I: Ethyl (4aS)-1-(4-fluorophenyl)-6-(p-tolylthio)-1,4,5,6,7,8-hexahydro-4aH-benzo[f]indazole-4a-carboxylate (1i)

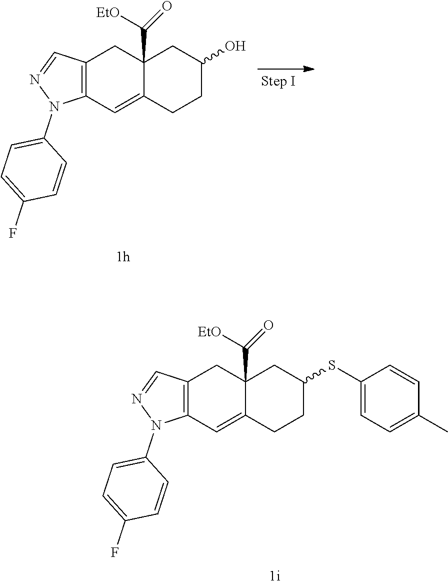 [SMILES: CCOC(=O)[C@]12Cc3cnn(-c4ccc(F)cc4)c3C=C1CCC(O)C2 ; CCOC(=O)[C@]12Cc3cnn(-c4ccc(F)cc4)c3C=C1CCC(Sc1ccc(C)cc1)C2]

Triethyl amine (682 mg, 6.75 mmol) and methanesulfonyl chloride (307 mg, 2.70 mmol) were added to a solution of ethyl (4aS,6S)-1-(4-fluorophenyl)-6-hydroxy-1,4,5,6,7,8-hexahydro-4aH-benzo[f]indazole-4a-carboxylate and methyl (4aS,6R)-1-(4-fluorophenyl)-6-hydroxy-1,4,5,6,7,8-hexahydro-4aH-benzo[f]indazole-4a-carboxylate (h) (800 mg, 2.25 mmol) in DCM (20 mL) at 0° C. After the mixture was stirred for 1 h, it was extracted with DCM. The combined organic layers were washed with brine and dried to afford crude product (100%), which was used for the next step without further purification.

Sodium methanolate (365 mg, 6.75 mmol) was added to a solution of 4-methylbenzenethiol (736 mg, 6.75 mmol) in DMF (10 ml) at rt. After the reaction mixture was stirred for 10 min, the mesylate prepared above in DMF (5 mL) was added and the resulting mixture was stirred at 80° C. for 2 h. The reaction mixture was cooled to rt, poured into water, and extracted with EtOAc. The combined organic layers were washed with brine and concentrated. The residue was purified by column chromatography on silica gel to afford the title compound (i) (580 mg, 56%) as a diastereometric mixture. m/z (ESI, +ve ion)=463.38[M+H]⁺.

### Step J: ((4aS,6S)-1-(4-Fluorophenyl)-6-(p-tolylthio)-1,4,5,6,7,8-hexahydro-4aH-benzo[f]indazol-4a-yl)(pyridin-2-yl)methanone (1j) and ((4aS,6R)-1-(4-fluorophenyl)-6-(p-tolylthio)-1,4,5,6,7,8-hexahydro-4aH-benzo[f]indazol-4a-yl)(pyridin-2-yl)methanone (2j)

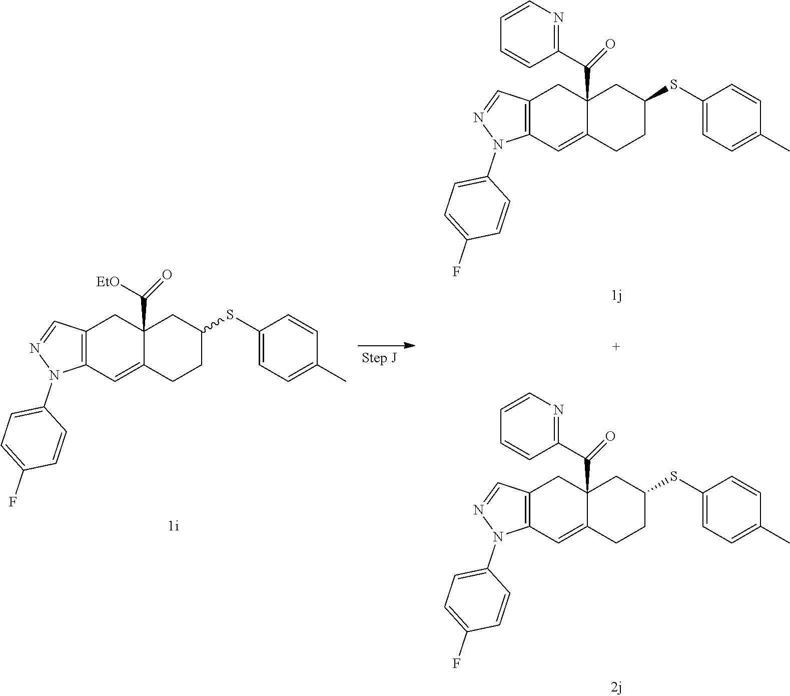 [SMILES: Cc1ccc(S[C@H]2CCC3=Cc4c(cnn4-c4ccc(F)cc4)C[C@]3(C(=O)c3ccccn3)C2)cc1 ; CCOC(=O)[C@]12Cc3cnn(-c4ccc(F)cc4)c3C=C1CCC(Sc1ccc(C)cc1)C2 ; Cc1ccc(S[C@@H]2CCC3=Cc4c(cnn4-c4ccc(F)cc4)C[C@]3(C(=O)c3ccccn3)C2)cc1]

2-Bromopyridine (405 mg, 2.58 mmol) was added directly to n-BuLi (1.6 M in hexane) in dry ether (5 mL) at −78° C. under N₂. After the reaction mixture was stirred at −78° C. for 45 min, a solution of ethyl (4aS,6S)-1-(4-fluorophenyl)-6-(p-tolylthio)-1,4,5,6,7,8-hexahydro-4aH-benzo[f]indazole-4a-carboxylate and methyl (4aS,6R)-1-(4-fluorophenyl)-6-(p-tolylthio)-1,4,5,6,7,8-hexahydro-4aH-benzo[f]indazole-4a-carboxylate (1i) (200 mg, 0.43 mmol) in dry ether (5 ml) was added dropwise. After being stirred at −78° C. for 30 min, the mixture was quenched with sat. NaHCO₃ and extracted with EtOAc. The combined organic layers were dried and concentrated under reduced pressure. The residue was purified by chromatography in silica gel to provide ((4aS,6S)-1-(4-fluorophenyl)-6-(p-tolylthio)-1,4,5,6,7,8-hexahydro-4aH-benzo[f]indazol-4a-yl)(pyridin-2-yl)methanone (1j) (100 mg, 47%, the less polar isomer) and ((4aS,6R)-1-(4-fluorophenyl)-6-(p-tolylthio)-1,4,5,6,7,8-hexahydro-4aH-benzo[f]indazol-4a-yl)(pyridin-2-yl)methanone (2j) (70 mg, 33%, the more polar isomer). m/z (ESI, +ve ion)=496.27[M+H]⁺.

### Step K: ((4aS,6S)-1-(4-Fluorophenyl)-6-tosyl-1,4,5,6,7,8-hexahydro-4aH-benzo[f]indazol-4a-yl)(pyridin-2-yl)methanone (1)

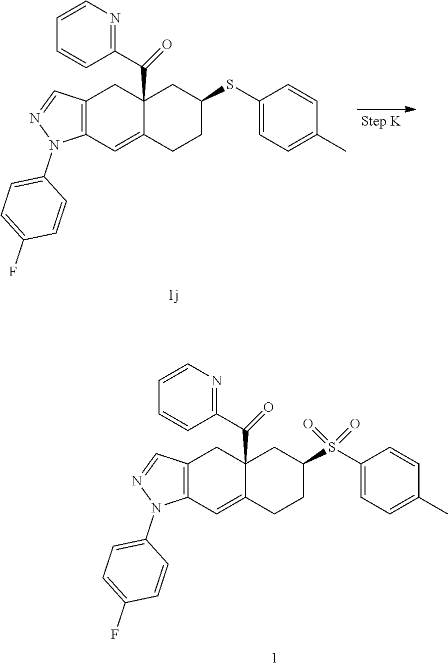 [SMILES: Cc1ccc(S[C@H]2CCC3=Cc4c(cnn4-c4ccc(F)cc4)C[C@]3(C(=O)c3ccccn3)C2)cc1 ; Cc1ccc(S(=O)(=O)[C@H]2CCC3=Cc4c(cnn4-c4ccc(F)cc4)C[C@]3(C(=O)c3ccccn3)C2)cc1]

3-Chlorobenzenecarboperoxoic acid (85%, 78 mg, 0.38 mmol) was added to a solution of ((4aS,6S)-1-(4-fluorophenyl)-6-(p-tolylthio)-1,4,5,6,7,8-hexahydro-4aH-benzo[f]indazol-4a-yl)(pyridin-2-yl)methanone (j) (95 mg, 0.19 mmol) in DCM (8 mL). After stirring at rt for 1 h, the reaction mixture was quenched with sat. NaHCO₃ and extracted with EtOAc. The organic layer was washed with brine, dried over MgSO₄, and concentrated in vacuo. The residue was purified by silica gel chromatography to give ((4aS,6S)-1-(4-fluorophenyl)-6-tosyl-1,4,5,6,7,8-hexahydro-4aH-benzo[f]indazol-4a-yl)(pyridin-2-yl)methanone (1) (37 mg, 37%) as a yellow solid. ¹H NMR (400 MHz, Chloroform-d) δ ppm 8.65 (1H, br d, J=4.4 Hz), 7.78-7.86 (2H, m), 7.75 (2H, br d, J=8.1 Hz), 7.38-7.51 (3H, m), 7.36 (2H, br d, J=8.1 Hz), 7.28 (1H, br s), 7.14 (2H, br t, J=8.5 Hz),6.44 (1H, s), 4.12 (1H, br d, J=16.5 Hz), 3.33-3.53 (1H, m), 3.12(1H, br d, J=13.3 Hz), 2.92 (1H, br d, J=16.5 Hz), 2.54-2.68 (1H, m), 2.47 (3H, s), 2.39-2.45 (2H, m), 2.07-2.20 (1H, m), 1.69-1.82 (1H, m); m/z (ESI, +ve ion)=528.3 [M+H]⁺.

### Example 2: ((4aS,6R)-1-(4-Fluorophenyl)-6-tosyl-1,4,5,6,7,8-hexahydro-4aH-benzo[f]indazol-4a-yl)(pyridin-2-yl)methanone (Compound 2)

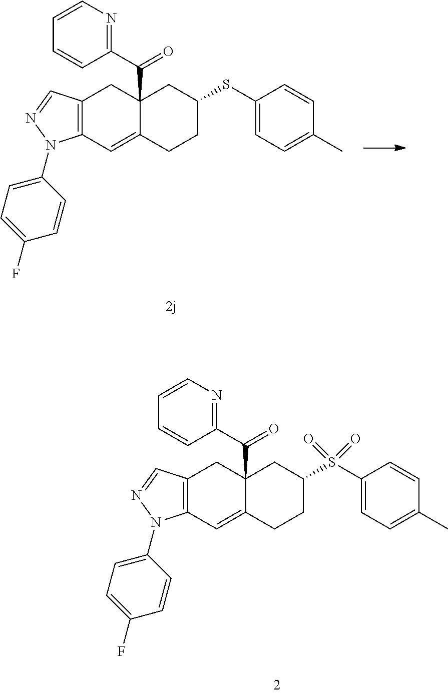 [SMILES: Cc1ccc(S[C@@H]2CCC3=Cc4c(cnn4-c4ccc(F)cc4)C[C@]3(C(=O)c3ccccn3)C2)cc1 ; Cc1ccc(S(=O)(=O)[C@@H]2CCC3=Cc4c(cnn4-c4ccc(F)cc4)C[C@]3(C(=O)c3ccccn3)C2)cc1]

3-Chlorobenzenecarboperoxoic acid (85%, 57 mg, 0.28 mmol) was added to a solution of ((4aS,6R)-1-(4-fluorophenyl)-6-(p-tolylthio)-1,4,5,6,7,8-hexahydro-4aH-benzo[f]indazol-4a-yl)(pyridin-2-yl)methanone (2j) (70 mg, 0.14 mmol) in DCM (8 mL) at rt. After the reaction was stirred for 1 h, it was quenched with sat. NaHCO₃ and extracted with EtOAc. The organic layer was washed with brine, dried over MgSO₄ and concentrated in vacuo. The residue was purified by silica gel chromatography to provide ((4aS,6R)-1-(4-fluorophenyl)-6-tosyl-1,4,5,6,7,8-hexahydro-4aH-benzo[f]indazol-4a-yl)(pyridin-2-yl)methanone (2) (38 mg, 53%) as a yellow solid. ¹H NMR (400 MHz, Chloroform-d) δ ppm 8.36 (1H, d, J=4.6 Hz), 7.79-7.85 (2H, m), 7.71 (2H, d, J=8.2 Hz), 7.52-7.58 (2H, m), 7.51 (1H, s), 7.42 (1H, br t, J=6.7 Hz), 7.33 (2H, br d, J=8.1 Hz), 7.24-7.26 (2H, m), 6.36 (1H, s), 3.97 (1H, d, J=16.9 Hz), 3.39 (1H, br d, J=12.8 Hz), 3.20 (1H, br t, J=12.3 Hz), 2.86 (1H, d, J=17.0 Hz), 2.55-2.65 (1H, m), 2.48 (3H, s), 2.24-2.38 (2H, m), 2.01 (1H, t, J=13.0 Hz), 1.73-1.86 (1H, m); m/z (ESI, +ve ion)=528.3 [M+H]⁺.

### Example 3: ((4aS,6S)-1-(4-Fluorophenyl)-6-((1-methyl-1H-pyrazol-4-yl)sulfonyl)-1,4,5,6,7,8-hexahydro-4aH-benzo[f]indazol-4a-yl)(4-(trifluoromethyl)pyridin-2-yl)methanone (3)

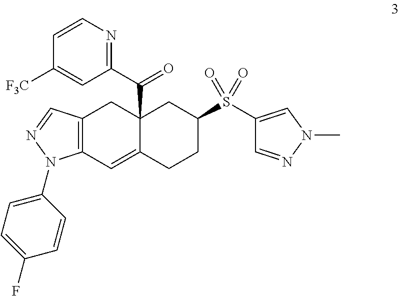 [SMILES: Cn1cc(S(=O)(=O)[C@H]2CCC3=Cc4c(cnn4-c4ccc(F)cc4)C[C@]3(C(=O)c3cc(C(F)(F)F)ccn3)C2)cn1]

### Step A: Ethyl (4aS,6R)-1-(4-fluorophenyl)-6-((methylsulfonyl)oxy)-1,4,5,6,7,8-hexahydro-4aH-benzo[f]indazole-4a-carboxylate (3a)

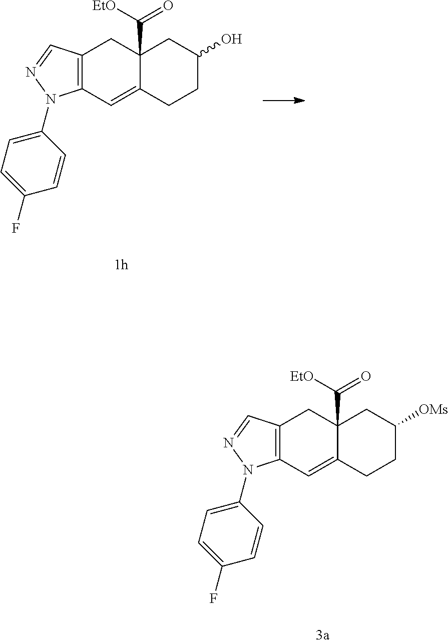 [SMILES: CCOC(=O)[C@]12Cc3cnn(-c4ccc(F)cc4)c3C=C1CCC(O)C2 ; CCOC(=O)[C@]12Cc3cnn(-c4ccc(F)cc4)c3C=C1CC[C@@H](OS(C)(=O)=O)C2]

To a solution of ethyl (4aS,6R)-1-(4-fluorophenyl)-6-hydroxy-5,6,7,8-tetrahydro-4H-benzo[f]indazole-4a-carboxylate (1h) (6.00 g, 16.8 mmol, trans:cis=60:40 from ¹HNMR, azeotroped with toluene) in DCM (150 mL) was added triethylamine (7.04 mL, 50.6 mmol). After the reaction mixture was cooled to 0° C., methanesulfonyl chloride (1.69 mL, 21.9 mmol) was added dropwise. The reaction was allowed to warm to rt and stirred at the same temperature for 1 h. The reaction was quenched with water and extracted with DCM (×2). The organics were washed with brine, dried over anhydrous sodium sulfate and concentrated under reduced pressure to afford an orange foaming solid, which was purified by silica gel chromatography (330 g SiO₂, 30%-100% EtOAc/hexanes, a gradient elution) to afford the title compound (trans isomer, R_{f}=0.42 in 50% EtOAc in hexanes, 3.90 g, 53%) (3a) and the other diastereomer (cis isomer, R_{f}=0.3 in 50% EtOAc in hexanes, 2.56 g, 35%) as white solids successively.

### Step B: Ethyl (4aS,6S)-1-(4-fluorophenyl)-6-((1-methyl-1H-pyrazol-4-yl)thio)-1,4,5,6,7,8-hexahydro-4aH-benzo[f]indazole-4a-carboxylate (3b)

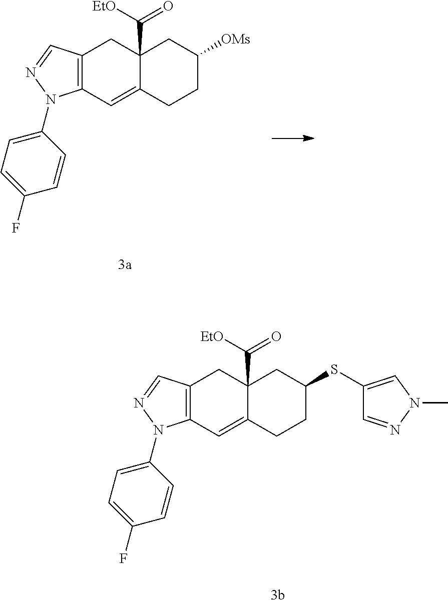 [SMILES: CCOC(=O)[C@]12Cc3cnn(-c4ccc(F)cc4)c3C=C1CC[C@@H](OS(C)(=O)=O)C2 ; CCOC(=O)[C@]12Cc3cnn(-c4ccc(F)cc4)c3C=C1CC[C@H](Sc1cnn(C)c1)C2]

To a stirred suspension of sodium hydride (60% in mineral oil, 120 mg, 3.0 mmol) in DMF (4 mL) was added 1-methylpyrazole-4-thiol (343 mg, 3.0 mmol) at rt under Ar. The mixture was stirred at rt until gas evolution was ceased (about 5 min). To the prepared thiolate solution was added a solution of ethyl (4aS,6R)-1-(4-fluorophenyl)-6-((methylsulfonyl)oxy)-1,4,5,6,7,8-hexahydro-4aH-benzo[f]indazole-4a-carboxylate (3a) (434 mg, 1 mmol) in DMF (2 mL) and the resulting mixture was heated at 55° C. for 1 h. After cooling to rt, the reaction mixture was poured into saturated aq. NH₄Cl solution and extracted (3×EtOAc). The combined organic layer was washed (water, 2×brine), dried (Na₂SO₄), and concentrated under reduced pressure. Purification of the residue by silica gel column chromatography (40 g SiO₂, 50% to 80% EtOAc/hexanes, a gradient elution) provided ethyl (4aS,6S)-1-(4-fluorophenyl)-6-((1-methyl-1H-pyrazol-4-yl)thio)-1,4,5,6,7,8-hexahydro-4aH-benzo[f]indazole-4a-carboxylate (3b) (319 mg, 70%) as a white foamy solid. m/z (ESI, +ve ion)=453.3 [M+H]⁺.

### Step C: ((4aS,6S)-1-(4-Fluorophenyl)-6-((1-methyl-1H-pyrazol-4-yl)thio)-1,4,5,6,7,8-hexahydro-4aH-benzo[f]indazol-4a-yl)methanol (3c)

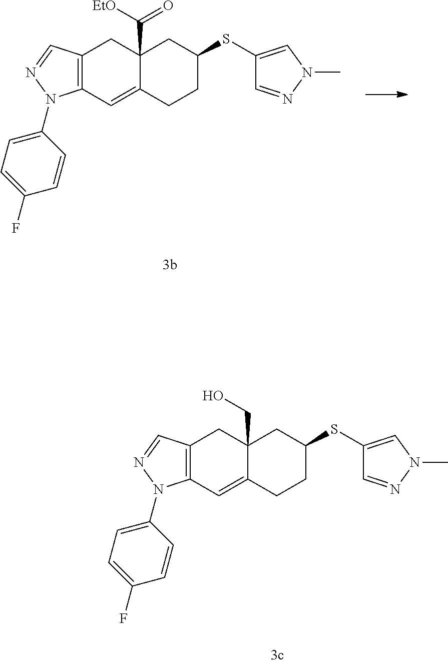 [SMILES: CCOC(=O)[C@]12Cc3cnn(-c4ccc(F)cc4)c3C=C1CC[C@H](Sc1cnn(C)c1)C2 ; Cn1cc(S[C@H]2CCC3=Cc4c(cnn4-c4ccc(F)cc4)C[C@]3(CO)C2)cn1]

To a stirred solution of ethyl (4aS,6S)-1-(4-fluorophenyl)-6-((1-methyl-H-pyrazol-4-yl)thio)-1,4,5,6,7,8-hexahydro-4aH-benzo[f]indazole-4a-carboxylate (3b) (319 mg, 0.70 mmol) in diethyl ether (10 mL) was added lithium aluminum hydride (1.0 M in THF, 0.85 mL, 0.85 mmol) at 0° C. Gas evolution was observed and the reaction mixture became white cloudy suspension. The mixture was stirred at 0° C. for 10 min, and then EtOAc (about 15 mL) was added until the reaction became a clear homogeneous. It was allowed to warm to rt and stirred for 20 min. The reaction turned into a white cloudy suspension and water was added. The suspension was filtered through a small pad of Celite and organic phase was washed (2×water, brine), dried (Na₂SO₄), and concentrated under reduced pressure to afford ((4aS,6S)-1-(4-fluorophenyl)-6-((1-methyl-1H-pyrazol-4-yl)thio)-1,4,5,6,7,8-hexahydro-4aH-benzo[f]indazol-4a-yl)methanol (3c) (290 mg, 100%) as a white solid. m/z (ESI, +ve ion)=411.1 [M+H]⁺.

### Step D: (4aS,6S)-1-(4-Fluorophenyl)-6-((1-methyl-1H-pyrazol-4-yl)thio)-1,4,5,6,7,8-hexahydro-4aH-benzo[f]indazole-4a-carbaldehyde (3d)

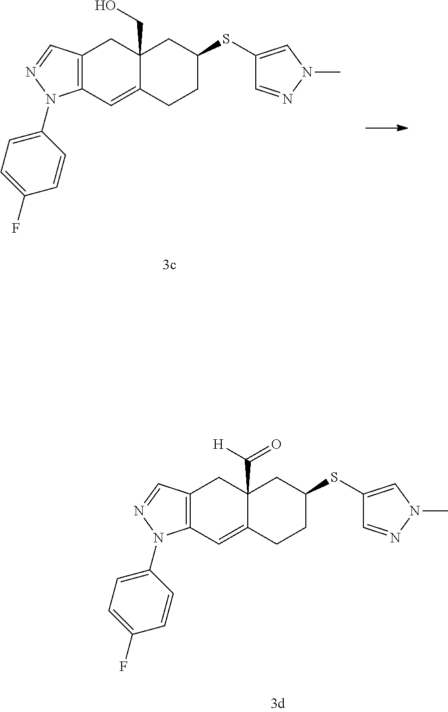 [SMILES: Cn1cc(S[C@H]2CCC3=Cc4c(cnn4-c4ccc(F)cc4)C[C@]3(CO)C2)cn1 ; Cn1cc(S[C@H]2CCC3=Cc4c(cnn4-c4ccc(F)cc4)C[C@]3(C=O)C2)cn1]

To a stirred solution of ((4aS,6S)-1-(4-fluorophenyl)-6-((1-methyl-1H-pyrazol-4-yl)thio)-1,4,5,6,7,8-hexahydro-4aH-benzo[f]indazol-4a-yl)methanol (290 mg, 0.70 mmol) (3c) in DCM (10 mL) was added Dess-Martin periodinane (314 mg, 0.74 mmol) at rt and the reaction mixture was stirred for 20 min. The reaction was quenched with saturated aq. NaHCO₃ solution and 10% aq. Na₂S₂O₃ solution. The mixture was stirred at rt for 15 min and extracted (3×DCM). The combined organic layer was washed (brine), dried (Na₂SO₄, and concentrated under reduced pressure. Purification of the residue by silica gel column chromatography (24 g SiO₂, 70% to 100% EtOAc/hexanes, a gradient elution) provided (4aS,6S)-1-(4-fluorophenyl)-6-((1-methyl-1H-pyrazol-4-yl)thio)-1,4,5,6,7,8-hexahydro-4aH-benzo[f]indazole-4a-carbaldehyde (3d) (231 mg, 80%) as a white solid. m/z (ESI, +ve ion)=409.1 [M+H]⁺.

### Step E: ((4aS,6S)-1-(4-Fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanol (3e)

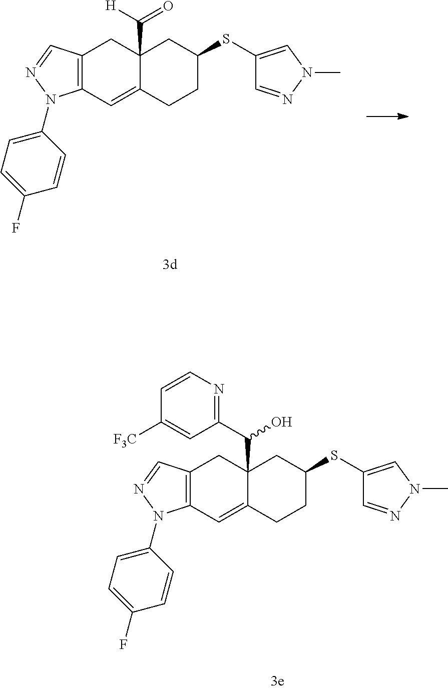 [SMILES: Cn1cc(S[C@H]2CCC3=Cc4c(cnn4-c4ccc(F)cc4)C[C@]3(C=O)C2)cn1 ; Cn1cc(S[C@H]2CCC3=Cc4c(cnn4-c4ccc(F)cc4)C[C@]3(C(O)c3cc(C(F)(F)F)ccn3)C2)cn1]

n-Butyllithium solution (1.6 Min hexane, 0.46 mL, 0.73 mmol) was added to a flask with diethyl ether (2.0 mL) at −78° C., followed by the dropwise addition of 2-bromo-4-(trifluoromethyl)pyridine (0.10 mL, 0.81 mmol), and the resulting mixture was stirred at −78° C. for 45 min. To the prepared aryllithium solution was added a solution of (4aS,6S)-1-(4-fluorophenyl)-6-((1-methyl-1H-pyrazol-4-yl)thio)-1,4,5,6,7,8-hexahydro-4aH-benzo[f]indazole-4a-carbaldehyde (3d) (50 mg, 0.12 mmol) in THF (1.2 mL) dropwise and stirred at −78° C. for 30 min. The reaction was quenched by the addition of water (8 mL). The dry ice bath was removed and the mixture was stirred for 10 min. Then a small amount of saturated aq. NH₄Cl solution was added and the solution was extracted (3×EtOAc). The combined organic layer was washed (brine), dried (Na₂SO₄), and concentrated under reduced pressure. Purification of the residue by silica gel column chromatography (12 g SiO₂, 1% to 3% MeOH/DCM, a gradient elution) provided ((4aS,6S)-1-(4-fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanol (3e) (43 mg, 63%) as an off-white solid. m/z (ESI, +ve ion) 556.2 [M+H]⁺.

### Step F: ((4aS,6S)-1-(4-Fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanone (3f)

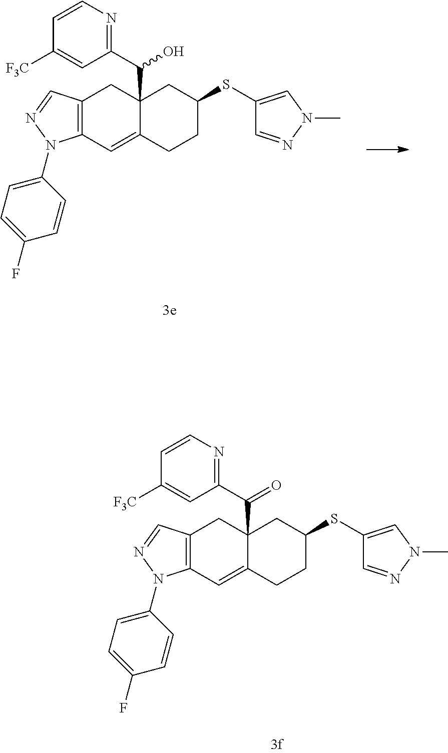 [SMILES: Cn1cc(S[C@H]2CCC3=Cc4c(cnn4-c4ccc(F)cc4)C[C@]3(C(O)c3cc(C(F)(F)F)ccn3)C2)cn1 ; Cn1cc(S[C@H]2CCC3=Cc4c(cnn4-c4ccc(F)cc4)C[C@]3(C(=O)c3cc(C(F)(F)F)ccn3)C2)cn1]

To a stirred solution of ((4aS,6S)-1-(4-fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanol (3e) (43 mg, 0.077 mmol) in DCM (1.5 mL) was added Dess-Martin periodinane (34.5 mg, 0.081 mmol) at rt. After the reaction mixture was stirred for 30 min, it was quenched with saturated aq. NaHCO₃ solution and 10% aq. Na₂S₂O₃ solution. The solution was stirred at rt for 15 min and extracted (3×DCM). The combined organic layer was washed (brine), dried (Na₂SO₄), and concentrated under reduced pressure. Purification of the residue by silica gel column chromatography (12 g SiO₂, 50% to 100% EtOAc/hexanes, a gradient elution) provided ((4aS,6S)-1-(4-fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanone (3f) (36 mg, 84%) as a white solid. m/z (ESI, +ve ion)=554.2 [M+H]⁺.

### Step G: ((4aS,6S)-1-(4-Fluorophenyl)-6-((1-methyl-1H-pyrazol-4-yl)sulfonyl)-1,4,5,6,7,8-hexahydro-4aH-benzo[f]indazol-4a-yl)(4-(trifluoromethyl)pyridin-2-yl)methanone (3)

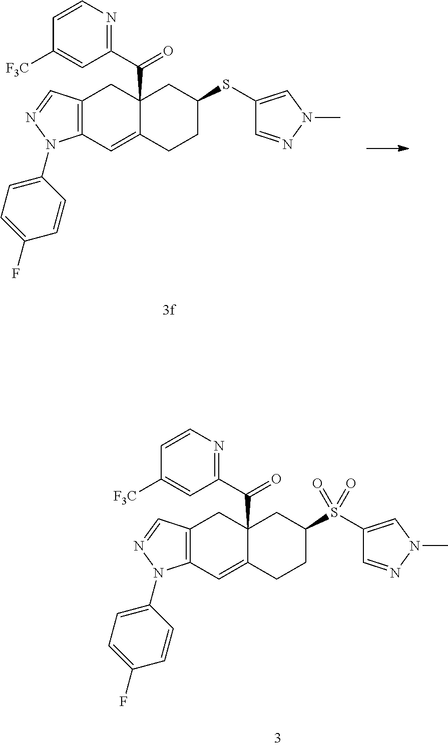 [SMILES: Cn1cc(S[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)[C@H]2CCC3=Cc4c(cnn4-c4ccc(F)cc4)C[C@]3(C(=O)c3cc(C(F)(F)F)ccn3)C2)cn1]

To a stirred solution of ((4aS,6S)-1-(4-fluorophenyl)-6-((1-methyl-1H-pyrazol-4-yl)thio)-1,4,5,6,7,8-hexahydro-4aH-benzo[f]indazol-4a-yl)(4-(trifluoromethyl)pyridin-2-yl)methanone (3f) (36 mg, 0.070 mmol) in DCM (3.8 mL) was added m-CPBA (75%,29.9 mg, 0.13 mmol) at 0° C. The resulting mixture was allowed to warm to rt, stirred for 20 min, quenched by saturated aq. NaHCO₃ solution, and extracted (3×DCM). The combined organic layer was washed (brine), dried (Na₂SO₄, and concentrated under reduced pressure. Purification of the residue by silica gel column chromatography (12 g SiO₂, 40% to 80% EtOAc/hexanes, a gradient elution) provided ((4aS,6S)-1-(4-fluorophenyl)-6-((1-methyl-1H-pyrazol-4-yl)sulfonyl)-1,4,5,6,7,8-hexahydro-4aH-benzo[f]indazol-4a-yl)(4-(trifluoromethyl)pyridin-2-yl)methanone (3) (25 mg, 66%) as an off-white solid. ¹H NMR (400 MHz, Chloroform-d) δ 8.89 (1H, d, J=5.2 Hz), 8.05 (1H, m), 7.82 (1H, s), 7.77 (1H, d, J=0.8 Hz), 7.42-7.39 (2H, m), 7.17-7.13 (2H, m), 6.48 (1H, d, J=1.6 Hz), 4.05 (1H, d, J=16.4 Hz), 3.98 (3H, s), 3.46-3.37 (1H, m), 3.12 (1H, dd, J=13.6, 2.6 Hz), 3.0 (1H, d, J=16.4 Hz), 2.63-2.41 (3H, m), 2.17-2.08 (1H, m), 1.97-1.88 (1H, m); m/z (ESI, +ve ion)=586.2 [M+H].

### Example 4: 4-Chloro-N-((4aS,6S)-1-(4-fluorophenyl)-4a-picolinoyl-4,4a,5,6,7,8-hexahydro-1H-benzo[f]indazol-6-yl)-N-methylbenzenesulfonamide (4)

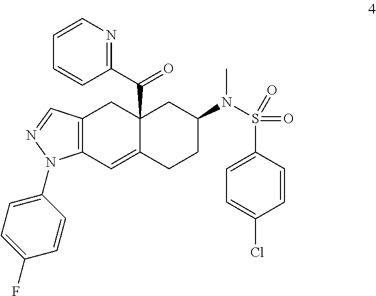 [SMILES: CN([C@H]1CCC2=Cc3c(cnn3-c3ccc(F)cc3)C[C@]2(C(=O)c2ccccn2)C1)S(=O)(=O)c1ccc(Cl)cc1]

### Step A: Methyl (4aS,6S)-6-azido-1-(4-fluorophenyl)-1,4,5,6,7,8-hexahydro-4aH-benzo[f]indazole-4a-carboxylate (4a)

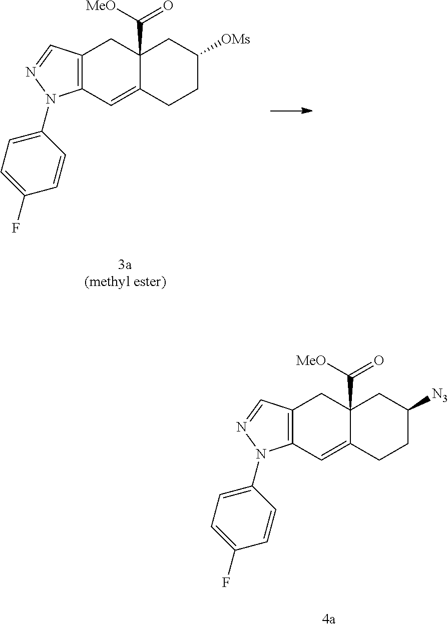 [SMILES: COC(=O)[C@]12Cc3cnn(-c4ccc(F)cc4)c3C=C1CC[C@@H](OS(C)(=O)=O)C2 ; COC(=O)[C@]12Cc3cnn(-c4ccc(F)cc4)c3C=C1CC[C@H](N=[N+]=[N-])C2]

Around bottom flask was charged with methyl (4aS,6R)-1-(4-fluorophenyl)-6-methylsulfonyloxy-5,6,7,8-tetrahydro-4H-benzo[f]indazole-4a-carboxylate (methyl ester of 3a) (290 mg, 0.69 mmol) and sodium azide (67 mg, 1.04 mmol), followed by anhydrous DMF (4 mL) under Ar. After the resulting mixture was heated at 90° C. overnight, it was quenched with water and EtOAc. The organic layer was washed with water and brine, dried with anhydrous sodium sulfate, and concentrated. The residue was purified by silica gel chromatography (12 g SiO₂, 0% to 25% EtOAc/hexanes, a gradient elution) to afford the title compound (4a) as a colorless oil (180 mg, 71%). m/z (ESI, +ve ion)=368.1 [M+H]⁺.

### Step B: Methyl (4aS,6S)-6-amino-1-(4-fluorophenyl)-1,4,5,6,7,8-hexahydro-4aH-benzo[f]indazole-4a-carboxylate (4b)

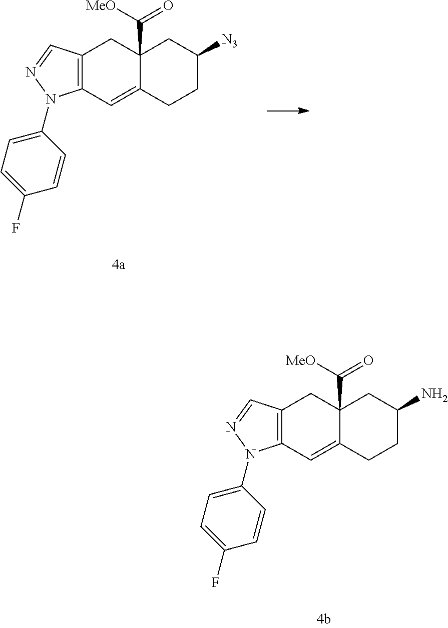 [SMILES: COC(=O)[C@]12Cc3cnn(-c4ccc(F)cc4)c3C=C1CC[C@H](N=[N+]=[N-])C2 ; COC(=O)[C@]12Cc3cnn(-c4ccc(F)cc4)c3C=C1CC[C@H](N)C2]

A flask was charged with methyl (4aS,6S)-6-azido-1-(4-fluorophenyl)-5,6,7,8-tetrahydro-4H-benzo[f]indazole-4a-carboxylate (4a) (592 mg, 1.61 mmol), zinc (422 mg, 6.45 mmol), ammonium formate (406 mg, 6.45 mmol), and anhydrous methanol (16 mL) successively. The reaction was stirred at rt for 50 min under nitrogen and additional zinc (422 mg) and ammonium formate (400 mg) were added. After the starting material was consumed (monitored by LCMS), the mixture was filtered through Celite and rinsed with MeOH and EtOAc. The organics were concentrated, diluted with water and extracted with 30% isopropanol in chloroform. The organic layer was dried with anhydrous sulfate and concentrated. The residue was purified (24 g SiO₂, 0%-75% EtOAc/hexanes and then 0%-5% MeOH/DCM+0.5% NH₄OH, a gradient elution) to afford the title compound (4b) (301 mg, 55%) as a white solid. m/z (ESI, +ve ion)=342.2 [M+H]⁺.

### Step C: Methyl (4aS,6S)-6-((4-chlorophenyl)sulfonamido)-1-(4-fluorophenyl)-1,4,5,6,7,8-hexahydro-4aH-benzo[f]indazole-4a-carboxylate (4c)

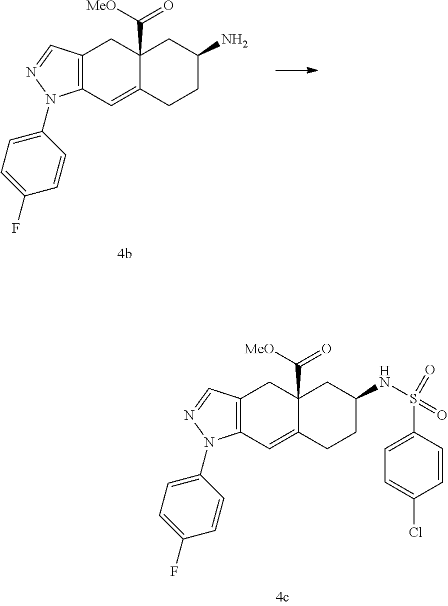 [SMILES: COC(=O)[C@]12Cc3cnn(-c4ccc(F)cc4)c3C=C1CC[C@H](N)C2 ; COC(=O)[C@]12Cc3cnn(-c4ccc(F)cc4)c3C=C1CC[C@H](NS(=O)(=O)c1ccc(Cl)cc1)C2]

Methyl(4aS,6S)-6-amino-1-(4-fluorophenyl)-5,6,7,8-tetrahydro-4H-benzo[f]indazole-4a-carboxylate (4b) (82 mg, 0.24 mmol) was azeotroped with toluene and flushed with argon. DCM (2 mL), pyridine (0.05 mL, 0.6 mmol), and 4-chlorobenzenesulfonyl chloride (81 mg, 0.38 mmol) were added successively. The reaction was stirred at rt for 3.5 h and concentrated. The residue was directly purified (12 g SiO₂, 0%-40% EtOAc/hexanes, a gradient elution) to afford the title compound (4c) as a colorless oil (57 mg, 46%). m/z (ESI, +ve ion)=516.0 [M+H]⁺.

### Step D: Methyl (4aS,6S)-6-((4-chloro-N-methylphenyl)sulfonamido)-1-(4-fluorophenyl)-1,4,5,6,7,8-hexahydro-4aH-benzo[f]indazole-4a-carboxylate (4d)

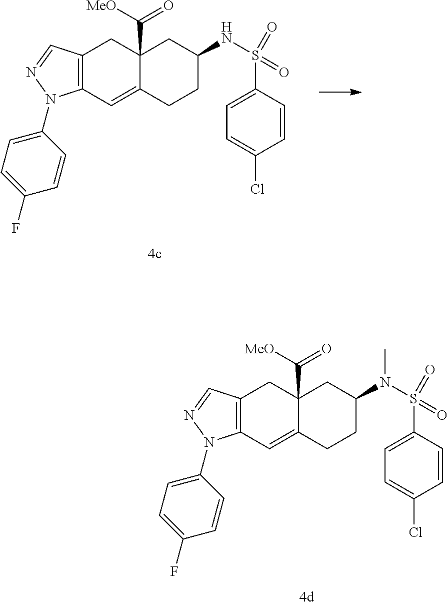 [SMILES: COC(=O)[C@]12Cc3cnn(-c4ccc(F)cc4)c3C=C1CC[C@H](NS(=O)(=O)c1ccc(Cl)cc1)C2 ; COC(=O)[C@]12Cc3cnn(-c4ccc(F)cc4)c3C=C1CC[C@H](N(C)S(=O)(=O)c1ccc(Cl)cc1)C2]

A flask was charged with methyl (4aS,6S)-6-[(4-chlorophenyl)sulfonylamino]-1-(4-fluorophenyl)-5,6,7,8-tetrahydro-4H-benzo[f]indazole-4a-carboxylate (4c) (57 mg, 0.11 mmol, azeotroped with toluene), sodium hydride (8.84 mg, 0.22 mmol) and followed by the addition of anhydrous DMF (1.5 mL). The reaction was stirred at rt for 10 min and followed by the addition of methyl iodide (0.03 mL, 0.55 mmol). After the reaction was stirred at rt for 2 h, it was quenched with water and saturated ammonium chloride, and extracted with EtOAc. The organics were dried with anhydrous sodium sulfate and concentrated. The residue was purified by silica gel column chromatography (12 g SiO₂, 0%-70% EtOAc/hexanes, a gradient elution) to afford the title compound (4d) as a white solid (59 mg, 100%). m/z (ESI, +ve ion)=530.2 [M+H]⁺.

### Step E: 4-Chloro-N-((4aS,6S)-1-(4-fluorophenyl)-4a-picolinoyl-4,4a,5,6,7,8-hexahydro-1H-benzo[f]indazol-6-yl)-N-methylbenzenesulfonamide (4)

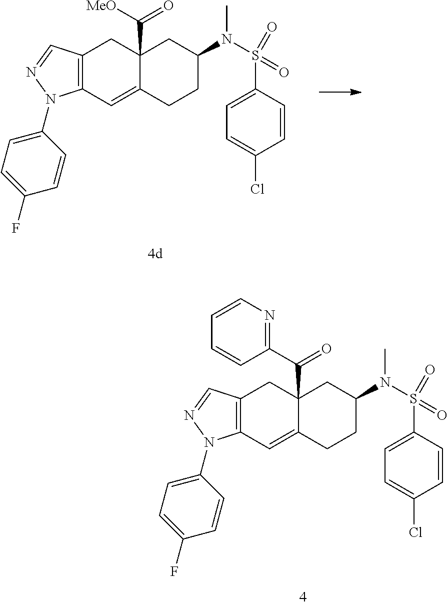 [SMILES: COC(=O)[C@]12Cc3cnn(-c4ccc(F)cc4)c3C=C1CC[C@H](N(C)S(=O)(=O)c1ccc(Cl)cc1)C2 ; CN([C@H]1CCC2=Cc3c(cnn3-c3ccc(F)cc3)C[C@]2(C(=O)c2ccccn2)C1)S(=O)(=O)c1ccc(Cl)cc1]

A 25 mL dry flask with methyl (4aS,6S)-6-[(4-chlorophenyl)sulfonyl-methyl-amino]-1-(4-fluorophenyl)-5,6,7,8-tetrahydro-4H-benzo[f]indazole-4a-carboxylate (4d) (59 mg, 0.11 mmol) was azeotroped with toluene and put on high vacuum pump for about 1 h. A separate dried flask under an argon balloon was charged with anhydrous ether (1.7 mL), cooled down to −78° C., n-butyllithium (0.28 mL, 0.45 mmol) was added, followed by the dropwise addition of 2-bromopyridine (0.05 mL, 0.51 mmol). The solution remained a brownish red and was stirred for 45 min at −78° C.

The flask with ethyl (4aS,6S)-6-(3,4-difluorophenyl)sulfanyl-1-(4-fluorophenyl)-5,6,7,8-tetrahydro-4H-benzo[f]indazole-4a-carboxylate (52. mg, 0.11 mmol) was flushed with argon and dissolved in anhydrous THF (0.3 mL) and anhydrous ether (0.9 mL). The resulting solution was added dropwise to the flask with the lithiated species at −78° C. The mixture was stirred for 50 min and then quenched with water and saturated NH₄Cl. The solution was allowed to warm to rt and extracted with EtOAc. The organic layer was separated, washed with brine, dried and concentrated. The residue was purified (12 g SiO₂, 0%-45% EtOAc/hexanes, a gradient elution) to afford the title compound (4) as a light yellow solid (47 mg, 72%). ¹H NMR (400 MHz, Chloroform-d) δ 8.55-8.59 (m, 1H), 7.79-7.84 (m, 2H), 7.73-7.79 (m, 2H), 7.55-7.61 (m, 2H), 7.40-7.47 (m, 3H), 7.29 (s, 1H), 7.13-7.21 (m, 2H), 6.46 (d, J=1.75 Hz, 1H), 4.35-4.46 (m, 1H), 3.91 (d, J=16.22 Hz, 1H), 3.07 (d, J=16.08 Hz, 1H), 2.79 (s, 3H), 2.52-2.63 (m, 1H), 2.40-2.48 (m, 1H), 2.30-2.37 (m, 1H), 2.15-2.24 (m, 1H), 1.74 (td, J=9.46, 4.17 Hz, 2H); m/z (ESI, +ve ion)=577.1 [M+H]⁺.

### Example 5: 4-Fluoro-N-((4aS,6S)-1-(4-fluorophenyl)-4a-(4-(trifluoromethyl)picolinoyl)-4,4a,5,6,7,8-hexahydro-1H-benzo[f]indazol-6-yl)-N-methylbenzenesulfonamide (5)

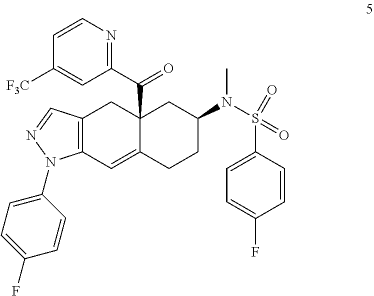 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccc(F)cc1]

### Step A: Methyl (4aS,6S)-1-(4-fluorophenyl)-6-(methylamino)-1,4,5,6,7,8-hexahydro-4aH-benzo[f]indazole-4a-carboxylate (5a)

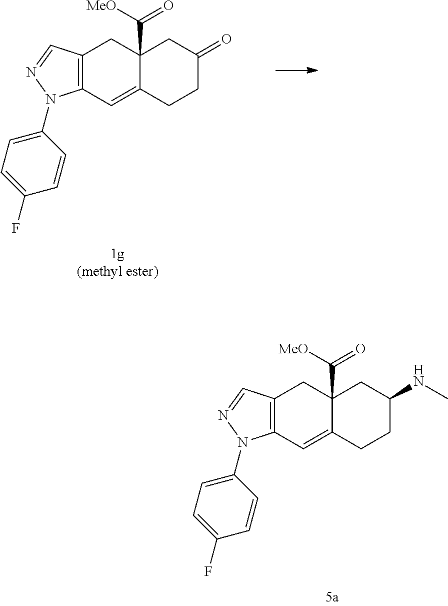 [SMILES: COC(=O)[C@@]12CC(=O)CCC1=Cc1c(cnn1-c1ccc(F)cc1)C2 ; CN[C@H]1CCC2=Cc3c(cnn3-c3ccc(F)cc3)C[C@]2(C(=O)OC)C1]

To a solution of methyl (4aS)-1-(4-fluorophenyl)-6-oxo-4,5,7,8-tetrahydrobenzo[f]indazole-4a-carboxylate (methyl ester of Ig) (422 mg, 1.24 mmol) and methylamine (0.92 mL, 7.44 mmol, 33% solution in ethanol) in DCE (6 mL) was added acetic acid (214.36 uL, 3.72 mmol). After the reaction was stirred for 2 min and cooled to 0° C., sodium triacetoxyborohydride (788 mg, 3.72 mmol) was added. After 5 min, the solution was allowed to warm to rt and the flask was sonicated for 2 min. After the reaction was stirred at rt for another 18 min, it was quenched (sat. aq. NaHCO₃) and extracted (2×EtOAc). The organic layers were washed (brine), dried (Na₂SO₄) and concentrated under reduced pressure. The crude product was purified (24 g SiO₂, 0%-75% EtOAc/hexanes and 0-6% gradient MeOH in DCM+0.5% NH₄OH, a gradient) to afford the title compound (5a) as a white solid (300 mg, 68%). m/z (ESI, +ve ion)=356.1 [M+H]⁺.

### Step B: Methyl (4aS,6S)-6-((4-fluoro-N-methylphenyl)sulfonamido)-1-(4-fluorophenyl)-1,4,5,6,7,8-hexahydro-4aH-benzo[f]indazole-4a-carboxylate (5b)

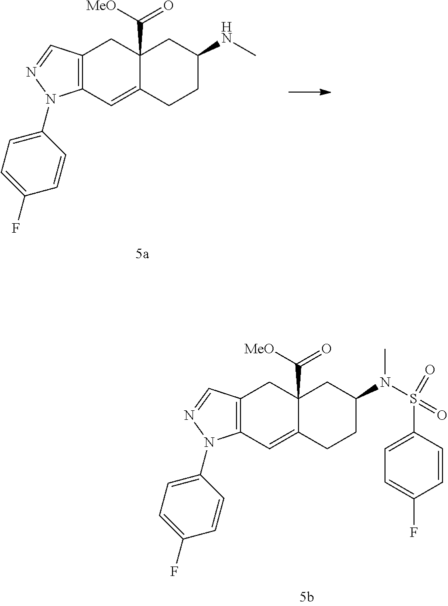 [SMILES: CN[C@H]1CCC2=Cc3c(cnn3-c3ccc(F)cc3)C[C@]2(C(=O)OC)C1 ; COC(=O)[C@]12Cc3cnn(-c4ccc(F)cc4)c3C=C1CC[C@H](N(C)S(=O)(=O)c1ccc(F)cc1)C2]

A flask was charged with methyl (4aS,6S)-1-(4-fluorophenyl)-6-(methylamino)-5,6,7,8-tetrahydro-4H-benzo[f]indazole-4a-carboxylate (5a) (60 mg, 0.17 mmol, azeotroped with toluene), 4-fluorobenzenesulfonyl chloride (65 mg, 0.34 mmol) was added, followed by the DCM (1 mL) and triethylamine (0.12 mL, 0.84 mmol) successively. The reaction was stirred under argon at rt for 3 h and then concentrated to dryness. The residue was directly purified (12 g SiO₂, 0%-35% EtOAc/hexanes, a gradient elution) to afford the title compound (5b) as a white solid (86 mg, 99%). m/z (ESI, +ve ion)=514.2 [M+H]⁺.

### Step C: 4-Fluoro-N-((4aS,6S)-1-(4-fluorophenyl)-4a-(4-(trifluoromethyl)picolinoyl)-4,4a,5,6,7,8-hexahydro-1H-benzo[f]indazol-6-yl)-N-methylbenzenesulfonamide (5)

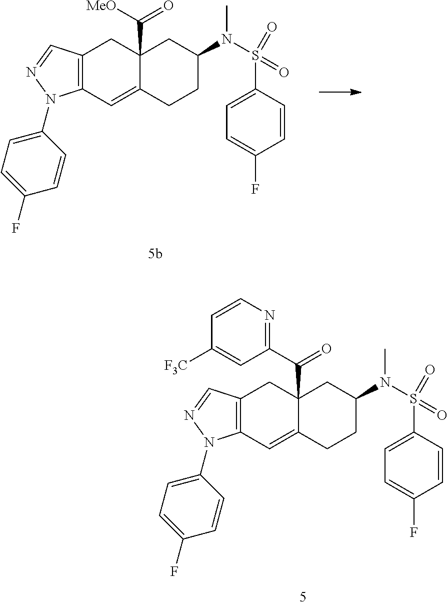 [SMILES: COC(=O)[C@]12Cc3cnn(-c4ccc(F)cc4)c3C=C1CC[C@H](N(C)S(=O)(=O)c1ccc(F)cc1)C2 ; CN([C@H]1CCC2=Cc3c(cnn3-c3ccc(F)cc3)C[C@]2(C(=O)c2cc(C(F)(F)F)ccn2)C1)S(=O)(=O)c1ccc(F)cc1]

A flask with methyl (4aS,6S)-1-(4-fluorophenyl)-6-[(4-fluorophenyl)sulfonyl-methyl-amino]-5,6,7,8-tetrahydro-4H-benzo[f]indazole-4a-carboxylate (5b) (44 mg, 0.09 mmol) was azeotroped with toluene and put on high vacuum. A separately dried flask under an argon balloon was charged with anhydrous ether (1.7 mL), cooled down to −78° C., n-butyllithium (0.24 mL, 0.39 mmol) was added, followed by the dropwise addition of 2-bromo-4-(trifluoromethyl)pyridine (0.05 mL, 0.43 mmol). The solution remained a brownish red and was stirred for 45 min at −78° C. The flask with the ester was flushed with argon and dissolved in anhydrous THF (0.2 mL) and anhydrous ether (0.5 mL). The resulting solution was added dropwise to the flask with the lithiated species at −78° C. The mixture was continuously stirred for 37 min and then quenched with water and saturated NH₄Cl. The solution was allowed to warm to rt and extracted with EtOAc. The organic layer was separated, washed with brine, dried and concentrated. The residue was purified (4 g SiO₂, 0%-30% EtOAc/hexanes, a gradient elution) to afford the title compound (5) as a light yellow solid (30 mg, 57%). ¹H NMR (400 MHz, Chloroform-d) δ 8.78 (d, J=4.97 Hz, 1H), 8.02 (s, 1H), 7.85-7.94 (m, 2H), 7.68 (dd, J=5.04, 1.10 Hz, 1H), 7.37-7.50 (m, 2H), 7.30 (s, 1H), 7.25-7.28 (m, 2H), 7.14-7.21 (m, 2H), 6.48 (s, 1H), 4.35-4.47 (m, 1H), 3.88 (d, J=16.37 Hz, 1H), 3.12 (d, J=16.52 Hz, 1H), 2.79 (s, 3H), 2.41-2.59 (m, 2H), 2.29-2.37 (m, 1H), 2.16-2.26 (m, 1H), 1.68-1.78 (m, 2H); m/z (ESI, +ve ion)=629.2 [M+H]⁺.

### Example 6: N-Cyclopropyl-N-((4aS,6S)-1-(4-fluorophenyl)-4a-picolinoyl-4,4a,5,6,7,8-hexahydro-1H-benzo[f]indazol-6-yl)benzamide (6)

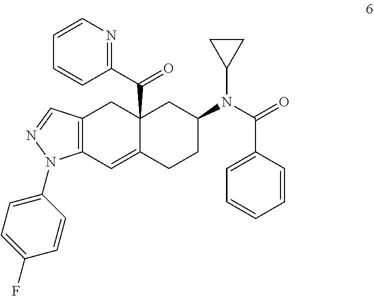 [SMILES: O=C(c1ccccc1)N(C1CC1)[C@H]1CCC2=Cc3c(cnn3-c3ccc(F)cc3)C[C@]2(C(=O)c2ccccn2)C1]

### Step A: Methyl (4aS,6S)-6-(cyclopropylamino)-1-(4-fluorophenyl)-1,4,5,6,7,8-hexahydro-4aH-benzo[f]indazole-4a-carboxylate (6a)

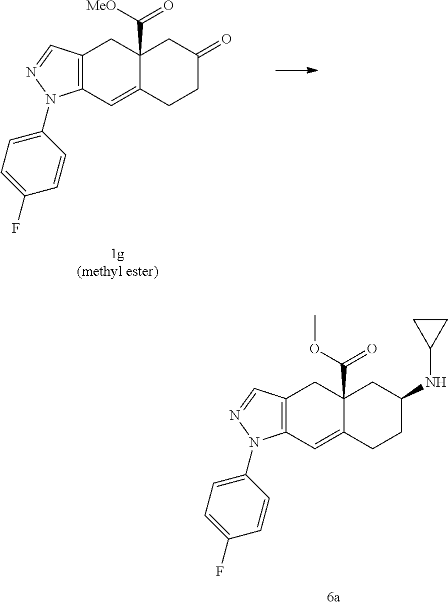 [SMILES: COC(=O)[C@@]12CC(=O)CCC1=Cc1c(cnn1-c1ccc(F)cc1)C2 ; COC(=O)[C@]12Cc3cnn(-c4ccc(F)cc4)c3C=C1CC[C@H](NC1CC1)C2]

Around bottom flask was charged with methyl (S)-1-(4-fluorophenyl)-6-oxo-1,4,5,6,7,8-hexahydro-4aH-benzo[f]indazole-4a-carboxylate (methyl ester of 1 g) (248 mg, 0.73 mmol) and added 3 mL of 1,2-dichloroethane under Ar. Addition of cyclopropanamine (151_L, 2.2 mmol) was followed, and the reaction solution was cooled to 0° C. Acetic acid (0.13 mL, 2.2 mmol) and sodium triacetoxyborohydride (463 mg, 2.2 mmol) was added thereafter and stirred at 0° C. After 5 min, the solution was allowed to warm to rt and allowed to stir. Consumption of the starting material was monitored by LCMS, and the reaction was completed after 3 h. The reaction was quenched with saturated aq. NaHCO₃ solution and extracted (2×DCM). The organic layers were washed (brine), dried (Na₂SO₄) and concentrated under reduced pressure to give methyl (4aS,6S)-6-(cyclopropylamino)-1-(4-fluorophenyl)-1,4,5,6,7,8-hexahydro-4aH-benzo[f]indazole-4a-carboxylate (6a) (278 mg) as a dark brown product. m/z (ESI, +ve ion)=382.3 [M+H]⁺.

### Step B: Methyl(4aS,6S)-6-(N-cyclopropylbenzamido)-1-(4-fluorophenyl)-1,4,5,6,7,8-hexahydro-4aH-benzo[f]indazole-4a-carboxylate (6b)

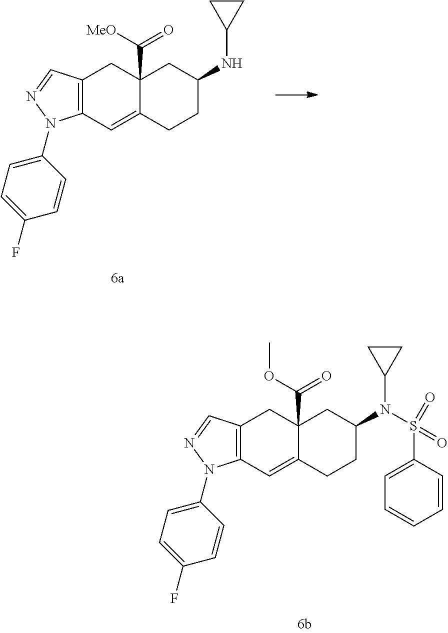 [SMILES: COC(=O)[C@]12Cc3cnn(-c4ccc(F)cc4)c3C=C1CC[C@H](NC1CC1)C2 ; COC(=O)[C@]12Cc3cnn(-c4ccc(F)cc4)c3C=C1CC[C@H](N(C1CC1)S(=O)(=O)c1ccccc1)C2]

To a solution of crude methyl (4aS,6S)-1-(4-fluorophenyl)-6-(methylamino)-1,4,5,6,7,8-hexahydro-4aH-benzo[f]indazole-4a-carboxylate (6a) (278 mg, 0.73 mmol) and benzoic acid (98 mg, 0.80 mmol) in DMF (3 mL) were added EDC (182 mg, 0.95 mmol), 3H-[1,2,3]triazolo[4,5-b]pyridin-3-ol (129 mg, 0.95 mmol), and sodium bicarbonate (122 mg, 1.46 mmol) successively. The reaction was allowed to stir overnight under Ar. The reaction was quenched with 10% aqueous citric acid and extracted (2×EtOAc). The organic layers were combined and washed (saturated aq. NaHCO₃ solution and brine), dried (Na₂SO₄, and concentrated under reduced pressure. The crude product was purified by silica gel chromatography (40 g SiO₂, 20% to 70% EtOAc/hexanes, a gradient elution) to provide methyl (4aS,6S)-6-(N-cyclopropylbenzamido)-1-(4-fluorophenyl)-1,4,5,6,7,8-hexahydro-4aH-benzo[f]indazole-4a-carboxylate (6b) (240 mg, 68%) as an off-white film. m/z (ESI, +ve ion)=486.2 [M+H]⁺.

### Step C: N-Cyclopropyl-N-((4aS,6S)-1-(4-fluorophenyl)-4a-picolinoyl-4,4a,5,6,7,8-hexahydro-1H-benzo[f]indazol-6-yl)benzamide (6)

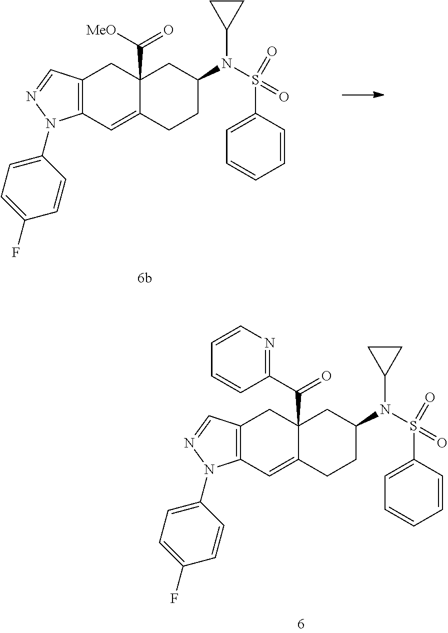 [SMILES: COC(=O)[C@]12Cc3cnn(-c4ccc(F)cc4)c3C=C1CC[C@H](N(C1CC1)S(=O)(=O)c1ccccc1)C2 ; O=C(c1ccccn1)[C@]12Cc3cnn(-c4ccc(F)cc4)c3C=C1CC[C@H](N(C1CC1)S(=O)(=O)c1ccccc1)C2]

To a round bottom flask was added ether (1.5 mL) and cooled to −78° C. under Ar. A solution of 0.16 M of n-BuLi in hexanes (0.44 mL, 0.70 mmol) was added to the flask followed by the addition of 2-bromopyridine (0.06 mL, 0.74 mmol) dropwise. The solution changed from yellow to dark maroon upon addition of 2-bromopyridine. The reaction solution was stirred at −78° C. for 30 min. Methyl (4aS)-6-[benzoyl(cyclopropyl)amino]-1-(4-fluorophenyl)-5,6,7,8-tetrahydro-4H-benzo[f]indazole-4a-carboxylate (6b) (38 mg, 0.08 mmol) previously azeotroped with toluene was dissolved with 0.75 mL ether under Ar. The methyl (4aS,6S)-6-[benzoyl(methyl)amino]-1-(4-fluorophenyl)-5,6,7,8-tetrahydro-4H-benzo[f]indazole-4a-carboxylate in ether was added dropwise to the lithiated species and allowed to stir at −78° C. under Ar for 30 min. The reaction was quenched by the addition of water and extracted (3×EtOAc), washed (brine), dried (Na₂SO₄) and concentrated under reduced pressure to give a dark orange oil. The crude product was purified by silica gel chromatography (4 g SiO₂, 0% to 50% EtOAc/Hexanes, a gradient elution) to provide N-cyclopropyl-N-((4aS,6S)-1-(4-fluorophenyl)-4a-picolinoyl-4,4a,5,6,7,8-hexahydro-1H-benzo[f]indazol-6-yl)benzamide (6) as a white solid (11 mg, 26%). ¹H NMR (400 MHz, Chloroform-d) δ 8.66 (1H, dt, J=1.9, 1.3 Hz), 7.77 (1H, d, J=1.32 Hz), 7.76 (1H, m), 7.49 (2H, m), 7.35-7.44 (6H, m), 7.26 (1H, br s), 7.14 (2H, t, J=8.33 Hz), 6.46 (1H, br d), 4.47 (1H, br m), 4.1 (1H, d, J=16.5 Hz), 3.23 (1H, br d, J=16.9 Hz), 2.91 (1H, t, J=13.2 Hz), 2.69-2.84 (3H, m), 2.50 (1H, m), 2.32 (1H, m), 2.00 (1H, m), 0.85 (2H, m), 0.56 (2H, m). m/z (ESI, +ve ion)=533.3 [M+H]⁺.

### Example 7: [(4aS,6S)-1-(4-Fluorophenyl)-6-(1-piperidylsulfonyl)-5,6,7,8-tetrahydro-4H-benzo[f]indazol-4a-yl]-(2-pyridyl)methanone (7)

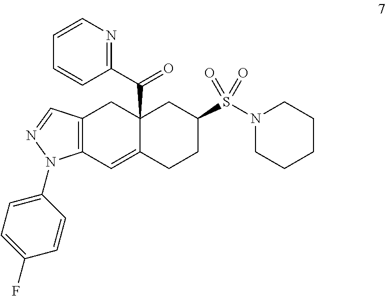 [SMILES: O=C(c1ccccn1)[C@]12Cc3cnn(-c4ccc(F)cc4)c3C=C1CC[C@H](S(=O)(=O)N1CCCCC1)C2]

### Step A: Ethyl (4aS,6S)-6-benzylsulfanyl-1-(4-fluorophenyl)-5,6,7,8-tetrahydro-4H-benzo[f]indazole-4a-carboxylate (7a)

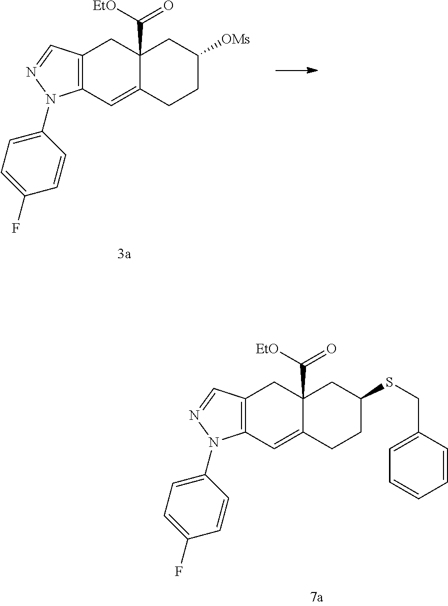 [SMILES: CCOC(=O)[C@]12Cc3cnn(-c4ccc(F)cc4)c3C=C1CC[C@@H](OS(C)(=O)=O)C2 ; CCOC(=O)[C@]12Cc3cnn(-c4ccc(F)cc4)c3C=C1CC[C@H](SCc1ccccc1)C2]

To a 20 mL vial was added sodium hydride (362 mg, 9.05 mmol) and DMF (2.0 mL). The resulting suspension was stirred at 0° C. for 2 min before the dropwise addition of benzylthiol (1.17 mL, 9.95 mmol). After the gas evolution stopped, ethyl (4aS,6R)-1-(4-fluorophenyl)-6-methylsulfonyloxy-5,6,7,8-tetrahydro-4H-benzo[f]indazole-4a-carboxylate (3a) (1.31 g, 3.02 mmol) in DMF (1 mL) was added and the reaction mixture was stirred at 55° C. until the starting material was fully consumed (monitored by LCMS). The residue was diluted with saturated aq. NH₄Cl solution and extracted (3×EtOAc). The combined organic layer was washed (brine), dried (Na₂SO₄, and concentrated under reduced pressure. The residue was purified by silica gel column chromatography (24 g SiO₂, 0% to 30% EtOAc/hexanes, a gradient elution) to provide ethyl (4aS,6S)-6-benzylsulfanyl-1-(4-fluorophenyl)-5,6,7,8-tetrahydro-4H-benzo[f]indazole-4a-carboxylate (7a) (721 mg, 52%) as a white foam. m/z (ESI, +ve ion)=463.3 [M+H]⁺.

### Step B: Ethyl (4aS,6S)-1-(4-fluorophenyl)-6-(1-piperidylsulfonyl)-5,6,7,8-tetrahydro-4H-benzo[f]indazole-4a-carboxylate (7b)

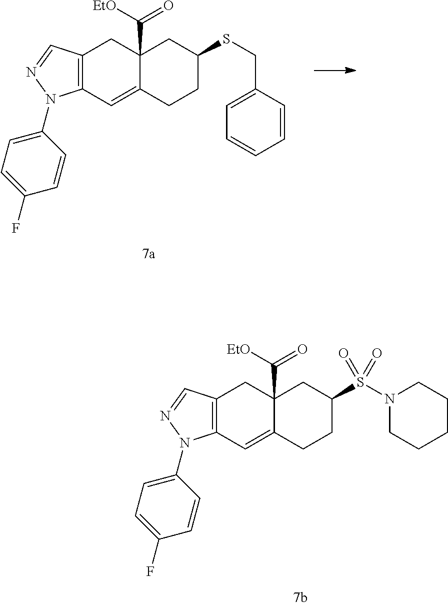 [SMILES: CCOC(=O)[C@]12Cc3cnn(-c4ccc(F)cc4)c3C=C1CC[C@H](SCc1ccccc1)C2 ; CCOC(=O)[C@]12Cc3cnn(-c4ccc(F)cc4)c3C=C1CC[C@H](S(=O)(=O)N1CCCCC1)C2]

To a solution of ethyl (4aS,6S)-6-benzylsulfanyl-1-(4-fluorophenyl)-5,6,7,8-tetrahydro-4H-benzo[f]indazole-4a-carboxylate (7a) (200 mg, 0.43 mmol) in ether (20 mL) was added iodosobenzene (285 mg, 1.3 mmol) at rt. The resulting suspension was stirred vigorously before the dropwise addition of conc. HCl (5.1 mL, 62 mmol). The reaction was continuously stirred until the starting material was almost consumed. The reaction was quenched with water and extracted (3×DCM). The combined organic layer was washed (brine), dried (Na₂SO₄, concentrated under reduced pressure, and dried under vacuum for 2 h. The crude material was then dissolved in DCM, stirred, and cooled to 0° C. for 5 min before piperidine (184 mg, 0.210 mL, 2.16 mmol) and N-ethyldiisopropylamine (279 mg. 0.380 mL, 2.16 mmol) were added successively. The reaction was stirred for 1 h while the sulfonyl chloride was fully consumed (monitored by LCMS). The residue was quenched with water and extracted (3×DCM). The combined organic layer was washed (brine), dried (Na₂SO₄), and concentrated under reduced pressure. The residue was purified by silica gel column chromatography (24 g SiO₂, 25% to 60% EtOAc/hexanes, a gradient elution) to provide ethyl (4aS,6S)-1-(4-fluorophenyl)-6-(1-piperidylsulfonyl)-5,6,7,8-tetrahydro-4H-benzo[f]indazole-4a-carboxylate (7b) (119 mg, 57%) as a yellow oil. m/z (ESI, +ve ion)=488.3 [M+H]⁺.

### Step C: [(4aS,6S)-1-(4-Fluorophenyl)-6-(1-piperidylsulfonyl)-5,6,7,8-tetrahydro-4H-benzo[f]indazol-4a-yl]-(2-pyridyl)methanone (7)

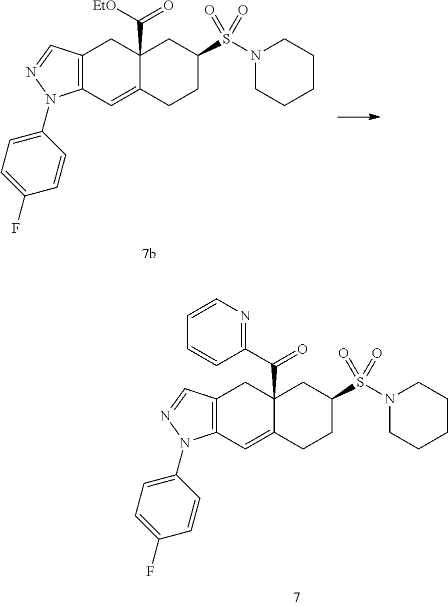 [SMILES: CCOC(=O)[C@]12Cc3cnn(-c4ccc(F)cc4)c3C=C1CC[C@H](S(=O)(=O)N1CCCCC1)C2 ; O=C(c1ccccn1)[C@]12Cc3cnn(-c4ccc(F)cc4)c3C=C1CC[C@H](S(=O)(=O)N1CCCCC1)C2]

A 10 mL dry vial was charged with diethyl ether (1 mL) and cooled down to −78° C. under argon. Upon stirring, n-butyllithium (0.15 mL, 0.38 mmol) was added dropwise and the resulting solution was stirred for 10 min before the dropwise addition of 2-bromopyridine (39 uL, 0.41 mmol). The red-brown solution was continuously stirred for another 45 min when ethyl (4aS,6S)-1-(4-fluorophenyl)-6-(1-piperidylsulfonyl)-5,6,7,8-tetrahydro-4H-benzo[f]indazole-4a-carboxylate (7b) (30.6 mg, 0.06 mmol) in THF (0.5 mL) was added dropwise. The reaction mixture was stirred for another 30 min (monitored by LCMS) and then quenched with small amount of water at −78° C., diluted (EtOAc), gradually raised to rt, diluted again (saturated aq. NaHCO₃ solution) and extracted (3×EtOAc). The combined organic layer was washed (brine), dried (Na₂SO₄) and concentrated under reduced pressure. The residue was purified by silica-gel column chromatography (12 g SiO₂, 10% to 40% Acetone/hexanes, a gradient elution) to provide [(4aS,6S)-1-(4-fluorophenyl)-6-(1-piperidylsulfonyl)-5,6,7,8-tetrahydro-4H-benzo[f]indazol-4a-yl]-(2-pyridyl)methanone (7) (9.2 mg, 28%) as a light yellow solid. ¹H NMR (400 MHz, Chloroform-d) δ 1.52-1.66 (m, 6H), 1.73-1.83 (m, 1H), 2.05 (t, J=12.94 Hz, 1H), 2.22-2.29 (m, 1H), 2.30-2.40 (m, 1H), 2.51-2.61 (m, 1H), 2.86 (d, J=16.66 Hz, 1H), 3.12-3.35 (m, 5H), 3.50 (dt, J=13.26, 2.58 Hz, 1H), 3.96 (d, J=16.66 Hz, 1H), 6.40 (d, J=1.61 Hz, 1H), 7.14-7.23 (m, 2H), 7.29 (s, 1H), 7.42-7.49 (m, 3H), 7.80-7.86 (m, 2H), 8.62 (dt, J=4.82, 1.24 Hz, 1H); m/z (ESI, +ve ion)=521.3 [M+H]⁺.

Examples 8-110: Examples 8-110 were prepared by similar procedures as described in Examples 1-7.

| | | | MS (ESI) |
|---|---|---|---|
| Ex | Name | Structure | [M + H]⁺ |
| 8 | ((4aS,6S)-1-(4-fluorophenyl)-6- tosyl-1,4,5,6,7,8-hexahydro- 4aH-benzo[f]indazol-4a- yl)(thiazol-2-yl)methanone | 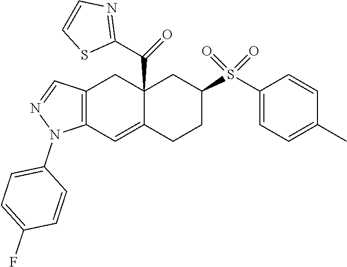 [SMILES: Cc1ccc(S(=O)(=O)[C@H]2CCC3=Cc4c(cnn4-c4ccc(F)cc4)C[C@]3(C(=O)c3nccs3)C2)cc1] | 534.3 |
| 9 | ((4aS,6R)-1-(4-fluorophenyl)-6- tosyl-1,4,5,6,7,8-hexahydro- 4aH-benzo[f]indazol-4a- yl)(thiazol-2-yl)methanone | 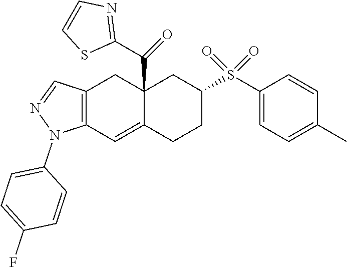 [SMILES: Cc1ccc(S(=O)(=O)[C@@H]2CCC3=Cc4c(cnn4-c4ccc(F)cc4)C[C@]3(C(=O)c3nccs3)C2)cc1] | 534.3 |
| 10 | ((4aS,6S)-1-(4-fluorophenyl)-6- ((4- (trifluoromethyl)phenyl)sulfonyl)- 1,4,5,6,7,8-hexahydro-4aH- benzo[f]indazol-4a-yl)(pyridin- 2-yl)methanone | 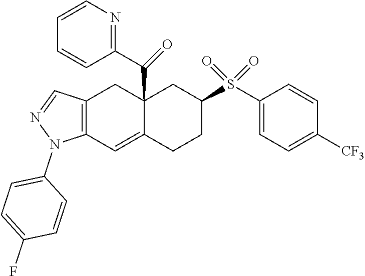 [SMILES: O=C(c1ccccn1)[C@]12Cc3cnn(-c4ccc(F)cc4)c3C=C1CC[C@H](S(=O)(=O)c1ccc(C(F)(F)F)cc1)C2] | 582.3 |
| 11 | ((4aS,6R)-1-(4-fluorophenyl)-6- ((4- (trifluoromethyl)phenyl)sulfonyl)- 1,4,5,6,7,8-hexahydro-4aH- benzo[f]indazol-4a-yl)(pyridin- 2-yl)methanone | 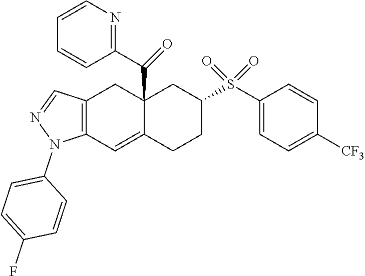 [SMILES: O=C(c1ccccn1)[C@]12Cc3cnn(-c4ccc(F)cc4)c3C=C1CC[C@@H](S(=O)(=O)c1ccc(C(F)(F)F)cc1)C2] | 582.3 |
| 12 | ((4aS,6S)-1-(4-fluorophenyl)-6- ((4- (trifluoromethyl)phenyl)sulfonyl)- 1,4,5,6,7,8-hexahydro-4aH- benzo[f]indazol-4a-yl)(thiazol- 2-yl)methanone | 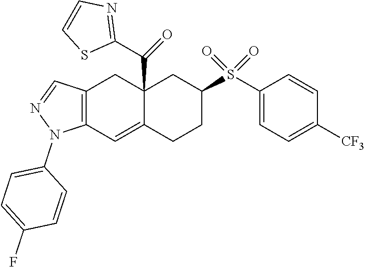 [SMILES: O=C(c1nccs1)[C@]12Cc3cnn(-c4ccc(F)cc4)c3C=C1CC[C@H](S(=O)(=O)c1ccc(C(F)(F)F)cc1)C2] | 588.3 |
| 13 | ((4aS,6R)-1-(4-fluorophenyl)-6- ((4- (trifluoromethyl)phenyl)sulfonyl)- 1,4,5,6,7,8-hexahydro-4aH- benzo[f]indazol-4a-yl)(thiazol- 2-yl)methanone | 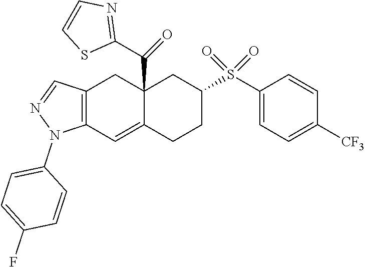 [SMILES: O=C(c1nccs1)[C@]12Cc3cnn(-c4ccc(F)cc4)c3C=C1CC[C@@H](S(=O)(=O)c1ccc(C(F)(F)F)cc1)C2] | 588.3 |
| 14 | ((4aS,6S)-6-((3,4- difluorophenyl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 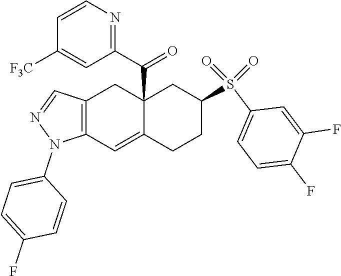 [SMILES: O=C(c1cc(C(F)(F)F)ccn1)[C@]12Cc3cnn(-c4ccc(F)cc4)c3C=C1CC[C@H](S(=O)(=O)c1ccc(F)c(F)c1)C2] | 618.2 |
| 15 | ((4aS,6S)-1-(4-fluorophenyl)-6- tosyl-1,4,5,6,7,8-hexahydro- 4aH-benzo[f]indazol-4a-yl)(4- (trifluoromethyl)pyridin-2- yl)methanone | 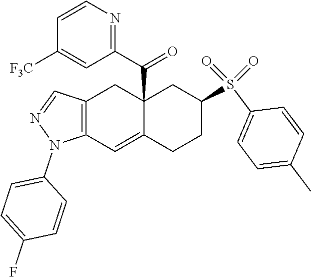 [SMILES: Cc1ccc(S(=O)(=O)[C@H]2CCC3=Cc4c(cnn4-c4ccc(F)cc4)C[C@]3(C(=O)c3cc(C(F)(F)F)ccn3)C2)cc1] | 596.2 |
| 16 | ((4aS,6R)-6-((3,4- difluorophenyl)sulfonyl)-1-(4- fluorophenyl)-1,4,5,6,7,8- hexahydro-4aH- benzo[f]indazol-4a-yl)(pyridin- 2-yl)methanone | 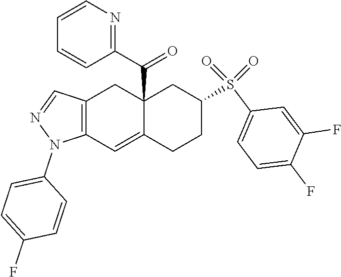 [SMILES: O=C(c1ccccn1)[C@]12Cc3cnn(-c4ccc(F)cc4)c3C=C1CC[C@@H](S(=O)(=O)c1ccc(F)c(F)c1)C2] | 550.2 |
| 17 | ((4aS,6R)-1-(4-fluorophenyl)-6- ((3- (trifluoromethyl)phenyl)sulfonyl)- 1,4,5,6,7,8-hexahydro-4aH- benzo[f]indazol-4a-yl)(4- (trifluoromethyl)pyridin-2- yl)methanone | 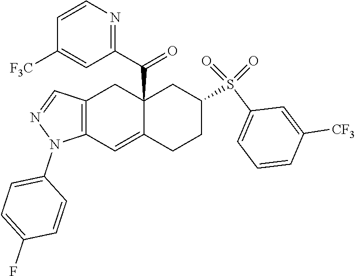 [SMILES: O=C(c1cc(C(F)(F)F)ccn1)[C@]12Cc3cnn(-c4ccc(F)cc4)c3C=C1CC[C@@H](S(=O)(=O)c1cccc(C(F)(F)F)c1)C2] | 650.2 |
| 18 | ((4aS,6R)-1-(4-fluorophenyl)-6- ((3- (trifluoromethyl)phenyl)sulfonyl)- 1,4,5,6,7,8-hexahydro-4aH- benzo[f]indazol-4a-yl)(pyridin- 2-yl)methanone | 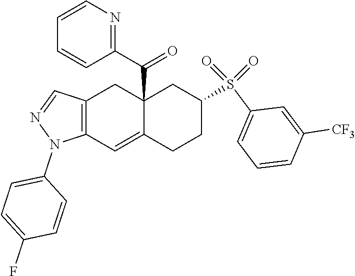 [SMILES: O=C(c1ccccn1)[C@]12Cc3cnn(-c4ccc(F)cc4)c3C=C1CC[C@@H](S(=O)(=O)c1cccc(C(F)(F)F)c1)C2] | 582.2 |
| 19 | ((4aS,6S)-1-(4-fluorophenyl)-6- ((4-methoxyphenyl)sulfonyl)- 1,4,5,6,7,8-hexahydro-4aH- benzo[f]indazol-4a-yl)(pyridin- 2-yl)methanone | 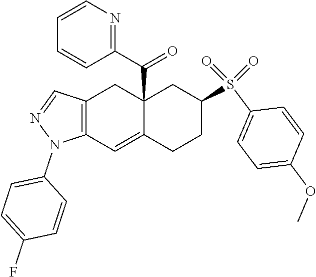 [SMILES: COc1ccc(S(=O)(=O)[C@H]2CCC3=Cc4c(cnn4-c4ccc(F)cc4)C[C@]3(C(=O)c3ccccn3)C2)cc1] | 544.3 |
| 20 | ((4aS,6S)-1-(4-fluorophenyl)-6- (phenylsulfonyl)-1,4,5,6,7,8- hexahydro-4aH- benzo[f]indazol-4a-yl)(pyridin- 2-yl)methanone | 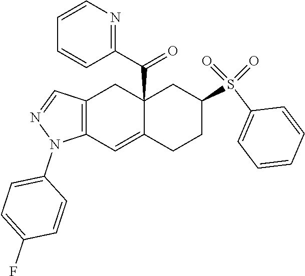 [SMILES: O=C(c1ccccn1)[C@]12Cc3cnn(-c4ccc(F)cc4)c3C=C1CC[C@H](S(=O)(=O)c1ccccc1)C2] | 514.1 |
| 21 | ((4aS,6S)-6-(benzylsulfonyl)-1- (4-fluorophenyl)-1,4,5,6,7,8- hexahydro-4aH- benzo[f]indazol-4a-yl)(pyridin- 2-yl)methanone | 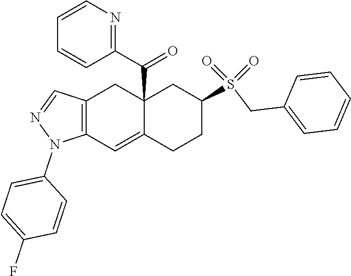 [SMILES: O=C(c1ccccn1)[C@]12Cc3cnn(-c4ccc(F)cc4)c3C=C1CC[C@H](S(=O)(=O)Cc1ccccc1)C2] | 528.2 |
| 22 | ((4aS,6S)-6-((3,4- difluorophenyl)sulfonyl)-1-(4- fluorophenyl)-1,4,5,6,7,8- hexahydro-4aH- benzo[f]indazol-4a-yl)(pyridin- 2-yl)methanone | 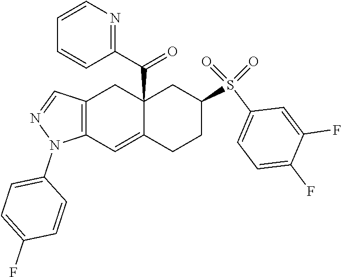 [SMILES: O=C(c1ccccn1)[C@]12Cc3cnn(-c4ccc(F)cc4)c3C=C1CC[C@H](S(=O)(=O)c1ccc(F)c(F)c1)C2] | 550.2 |
| 23 | ((4aS,6S)-1-(4-fluorophenyl)-6- ((1-methyl-1H-pyrazol-4- yl)sulfonyl)-1,4,5,6,7,8- hexahydro-4aH- benzo[f]indazol-4a-yl)(4- methylpyridin-2-yl)methanone | 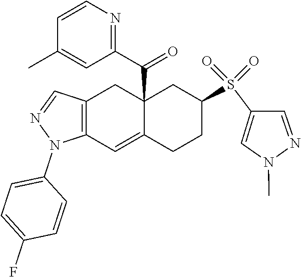 [SMILES: Cc1ccnc(C(=O)[C@]23Cc4cnn(-c5ccc(F)cc5)c4C=C2CC[C@H](S(=O)(=O)c2cnn(C)c2)C3)c1] | 532.2 |
| 24 | ((4aS,6R)-1-(4-fluorophenyl)-6- (phenylsulfonyl)-1,4,5,6,7,8- hexahydro-4aH- benzo[f]indazol-4a-yl)(pyridin- 2-yl)methanone | 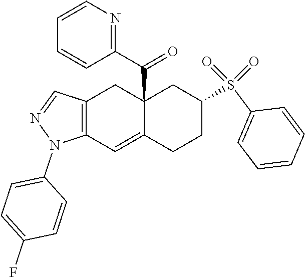 [SMILES: O=C(c1ccccn1)[C@]12Cc3cnn(-c4ccc(F)cc4)c3C=C1CC[C@@H](S(=O)(=O)c1ccccc1)C2] | 514.2 |
| 25 | ((4aS,6S)-1-(4-fluorophenyl)-6- ((4-fluorophenyl)sulfonyl)- 1,4,5,6,7,8-hexahydro-4aH- benzo[f]indazol-4a-yl)(pyridin- 2-yl)methanone | 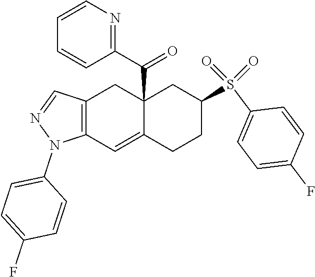 [SMILES: O=C(c1ccccn1)[C@]12Cc3cnn(-c4ccc(F)cc4)c3C=C1CC[C@H](S(=O)(=O)c1ccc(F)cc1)C2] | 532.2 |
| 26 | ((4aS,6S)-1-(4-fluorophenyl)-6- (pyridin-2-ylsulfonyl)- 1,4,5,6,7,8-hexahydro-4aH- benzo[f]indazol-4a-yl)(pyridin- 2-yl)methanone | 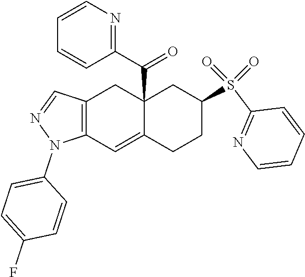 [SMILES: O=C(c1ccccn1)[C@]12Cc3cnn(-c4ccc(F)cc4)c3C=C1CC[C@H](S(=O)(=O)c1ccccn1)C2] | 515.2 |
| 27 | ((4aS,6R)-6-((1,2,3-thiadiazol- 5-yl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 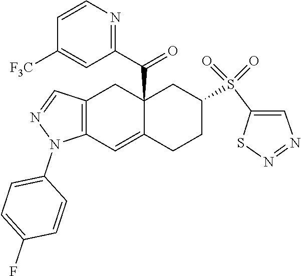 [SMILES: O=C(c1cc(C(F)(F)F)ccn1)[C@]12Cc3cnn(-c4ccc(F)cc4)c3C=C1CC[C@@H](S(=O)(=O)c1cnns1)C2] | 590.2 |
| 28 | ((4aS,6S)-1-(4-fluorophenyl)-6- ((3- (trifluoromethyl)phenyl)sulfonyl)- 1,4,5,6,7,8-hexahydro-4aH- benzo[f]indazol-4a-yl)(pyridin- 2-yl)methanone | 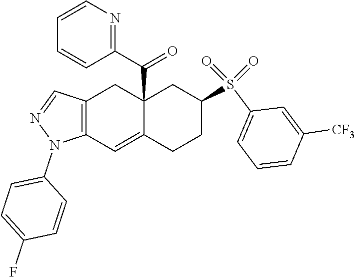 [SMILES: O=C(c1ccccn1)[C@]12Cc3cnn(-c4ccc(F)cc4)c3C=C1CC[C@H](S(=O)(=O)c1cccc(C(F)(F)F)c1)C2] | 582.2 |
| 29 | ((4aS,6R)-1-(4-fluorophenyl)-6- ((4-fluorophenyl)sulfonyl)- 1,4,5,6,7,8-hexahydro-4aH- benzo[f]indazol-4a-yl)(pyridin- 2-yl)methanone | 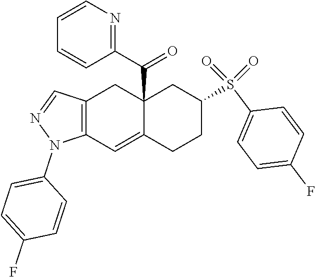 [SMILES: O=C(c1ccccn1)[C@]12Cc3cnn(-c4ccc(F)cc4)c3C=C1CC[C@@H](S(=O)(=O)c1ccc(F)cc1)C2] | 532.2 |
| 30 | ((4aS,6S)-6-((3,4- difluorophenyl)sulfonyl)-1-(4- fluorophenyl)-1,4,5,6,7,8- hexahydro-4aH- benzo[f]indazol-4a-yl)(4- methylpyridin-2-yl)methanone | 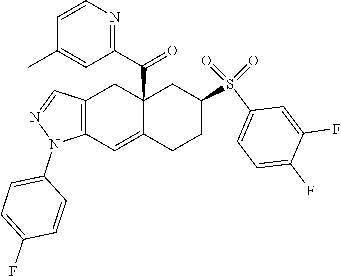 [SMILES: Cc1ccnc(C(=O)[C@]23Cc4cnn(-c5ccc(F)cc5)c4C=C2CC[C@H](S(=O)(=O)c2ccc(F)c(F)c2)C3)c1] | 564.2 |
| 31 | ((4aS,6S)-1-(4-fluorophenyl)-6- ((3- (trifluoromethyl)phenyl)sulfonyl)- 1,4,5,6,7,8-hexahydro-4aH- benzo[f]indazol-4a-yl)(5- methylpyridin-2-yl)methanone | 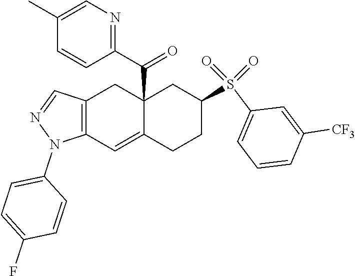 [SMILES: Cc1ccc(C(=O)[C@]23Cc4cnn(-c5ccc(F)cc5)c4C=C2CC[C@H](S(=O)(=O)c2cccc(C(F)(F)F)c2)C3)nc1] | 596.2 |
| 32 | ((4aS,6S)-1-(4-fluorophenyl)-6- ((5-(trifluoromethyl)pyridin-2- yl)sulfonyl)-1,4,5,6,7,8- hexahydro-4aH- benzo[f]indazol-4a-yl)(pyridin- 2-yl)methanone | 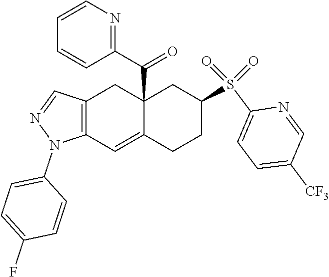 [SMILES: O=C(c1ccccn1)[C@]12Cc3cnn(-c4ccc(F)cc4)c3C=C1CC[C@H](S(=O)(=O)c1ccc(C(F)(F)F)cn1)C2] | 583.1 |
| 33 | ((4aS,6S)-1-(4-fluorophenyl)-6- ((2-methyl-2H-1,2,3-triazol-4- yl)sulfonyl)-1,4,5,6,7,8- hexahydro-4aH- benzo[f]indazol-4a-yl)(4- (trifluoromethyl)pyridin-2- yl)methanone | 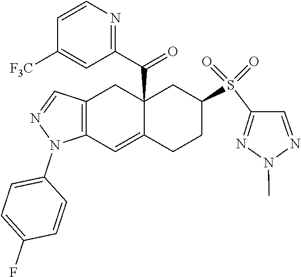 [SMILES: Cn1ncc(S(=O)(=O)[C@H]2CCC3=Cc4c(cnn4-c4ccc(F)cc4)C[C@]3(C(=O)c3cc(C(F)(F)F)ccn3)C2)n1] | 587.2 |
| 34 | ((4aS,6S)-1-(4-fluorophenyl)-6- (pyridin-2-ylsulfonyl)- 1,4,5,6,7,8-hexahydro-4aH- benzo[f]indazol-4a-yl)(4- methylpyridin-2-yl)methanone | 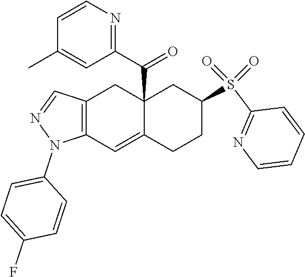 [SMILES: Cc1ccnc(C(=O)[C@]23Cc4cnn(-c5ccc(F)cc5)c4C=C2CC[C@H](S(=O)(=O)c2ccccn2)C3)c1] | 529.2 |
| 35 | ((4aS,6S)-6- (cyclohexylsulfonyl)-1-(4- fluorophenyl)-1,4,5,6,7,8- hexahydro-4aH- benzo[f]indazol-4a-yl)(pyridin- 2-yl)methanone | 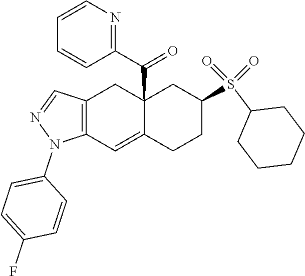 [SMILES: O=C(c1ccccn1)[C@]12Cc3cnn(-c4ccc(F)cc4)c3C=C1CC[C@H](S(=O)(=O)C1CCCCC1)C2] | 520.2 |
| 36 | ((4aS,6S)-1-(4-fluorophenyl)-6- (phenylsulfonyl)-1,4,5,6,7,8- hexahydro-4aH- benzo[f]indazol-4a-yl)(4- methylpyridin-2-yl)methanone | 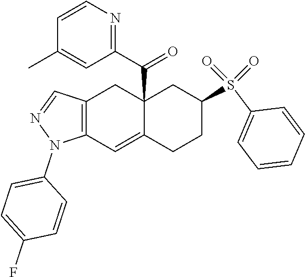 [SMILES: Cc1ccnc(C(=O)[C@]23Cc4cnn(-c5ccc(F)cc5)c4C=C2CC[C@H](S(=O)(=O)c2ccccc2)C3)c1] | 528.2 |
| 37 | ((4aS,6S)-6-((3- chlorophenyl)sulfonyl)-1-(4- fluorophenyl)-1,4,5,6,7,8- hexahydro-4aH- benzo[f]indazol-4a-yl)(pyridin- 2-yl)methanone | 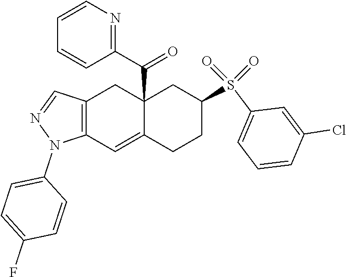 [SMILES: O=C(c1ccccn1)[C@]12Cc3cnn(-c4ccc(F)cc4)c3C=C1CC[C@H](S(=O)(=O)c1cccc(Cl)c1)C2] | 548.2 |
| 38 | ((4aS,6R)-1-(4-fluorophenyl)-6- (phenylsulfonyl)-1,4,5,6,7,8- hexahydro-4aH- benzo[f]indazol-4a-yl)(4- methylpyridin-2-yl)methanone | 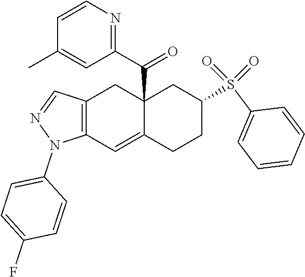 [SMILES: Cc1ccnc(C(=O)[C@]23Cc4cnn(-c5ccc(F)cc5)c4C=C2CC[C@@H](S(=O)(=O)c2ccccc2)C3)c1] | 528.2 |
| 39 | ((4aS,6S)-1-(4-fluorophenyl)-6- ((2-methyl-2H-1,2,3-triazol-4- yl)sulfonyl)-1,4,5,6,7,8- hexahydro-4aH- benzo[f]indazol-4a-yl)(4- methylpyridin-2-yl)methanone | 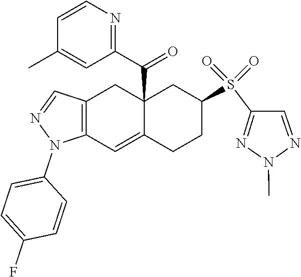 [SMILES: Cc1ccnc(C(=O)[C@]23Cc4cnn(-c5ccc(F)cc5)c4C=C2CC[C@H](S(=O)(=O)c2cnn(C)n2)C3)c1] | 533.2 |
| 40 | ((4aS,6S)-6-(tert- butylsulfonyl)-1-(4- fluorophenyl)-1,4,5,6,7,8- hexahydro-4aH- benzo[f]indazol-4a-yl)(pyridin- 2-yl)methanone | 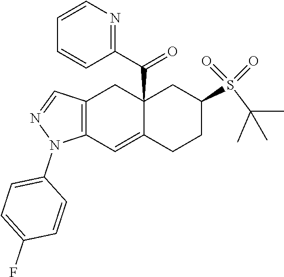 [SMILES: CC(C)(C)S(=O)(=O)[C@H]1CCC2=Cc3c(cnn3-c3ccc(F)cc3)C[C@]2(C(=O)c2ccccn2)C1] | 494.2 |
| 41 | ((4aS,6S)-1-(4-fluorophenyl)-6- (pyridin-2-ylsulfonyl)- 1,4,5,6,7,8-hexahydro-4aH- benzo[f]indazol-4a-yl)(4- (trifluoromethyl)pyridin-2- yl)methanone | 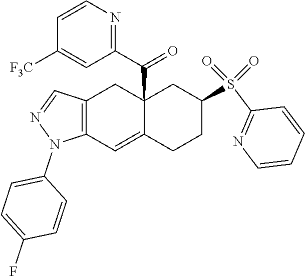 [SMILES: O=C(c1cc(C(F)(F)F)ccn1)[C@]12Cc3cnn(-c4ccc(F)cc4)c3C=C1CC[C@H](S(=O)(=O)c1ccccn1)C2] | 583.2 |
| 42 | ((4aS,6S)-1-(4-fluorophenyl)-6- (pyridin-2-ylsulfonyl)- 1,4,5,6,7,8-hexahydro-4aH- benzo[f]indazol-4a-yl)(5- methylpyridin-2-yl)methanone | 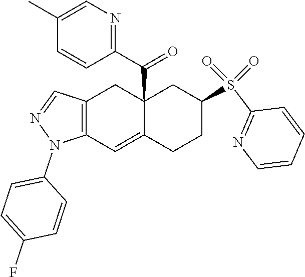 [SMILES: Cc1ccc(C(=O)[C@]23Cc4cnn(-c5ccc(F)cc5)c4C=C2CC[C@H](S(=O)(=O)c2ccccn2)C3)nc1] | 529.3 |
| 43 | ((4aS,6S)-1-(4-fluorophenyl)-6- ((4-(trifluoromethyl)pyridin-2- yl)sulfonyl)-1,4,5,6,7,8- hexahydro-4aH- benzo[f]indazol-4a-yl)(pyridin- 2-yl)methanone | 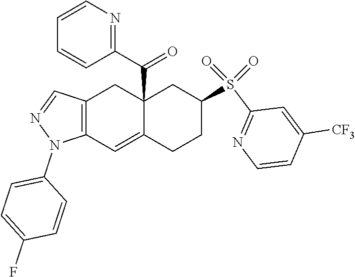 [SMILES: O=C(c1ccccn1)[C@]12Cc3cnn(-c4ccc(F)cc4)c3C=C1CC[C@H](S(=O)(=O)c1cc(C(F)(F)F)ccn1)C2] | 583.2 |
| 44 | ((4aS,6S)-6-((3- chlorophenyl)sulfonyl)-1-(4- fluorophenyl)-1,4,5,6,7,8- hexahydro-4aH- benzo[f]indazol-4a-yl)(4- methylpyridin-2-yl)methanone | 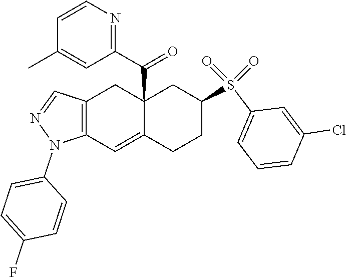 [SMILES: Cc1ccnc(C(=O)[C@]23Cc4cnn(-c5ccc(F)cc5)c4C=C2CC[C@H](S(=O)(=O)c2cccc(Cl)c2)C3)c1] | 562.2 |
| 45 | ((4aS,6S)-1-(4-fluorophenyl)-6- (phenylsulfonyl)-1,4,5,6,7,8- hexahydro-4aH- benzo[f]indazol-4a-yl)(5- methylpyridin-2-yl)methanone | 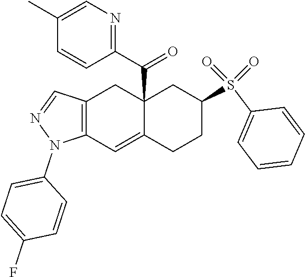 [SMILES: Cc1ccc(C(=O)[C@]23Cc4cnn(-c5ccc(F)cc5)c4C=C2CC[C@H](S(=O)(=O)c2ccccc2)C3)nc1] | 528.2 |
| 46 | ((4aS,6S)-1-(4-fluorophenyl)-6- ((tetrahydro-2H-pyran-4- yl)sulfonyl)-1,4,5,6,7,8- hexahydro-4aH- benzo[f]indazol-4a-yl)(pyridin- 2-yl)methanone | 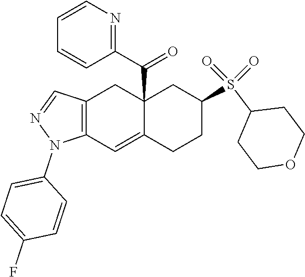 [SMILES: O=C(c1ccccn1)[C@]12Cc3cnn(-c4ccc(F)cc4)c3C=C1CC[C@H](S(=O)(=O)C1CCOCC1)C2] | 522.3 |
| 47 | ((4aS,6S)-1-(4-fluorophenyl)-6- (pyridin-4-ylsulfonyl)- 1,4,5,6,7,8-hexahydro-4aH- benzo[f]indazol-4a-yl)(pyridin- 2-yl)methanone | 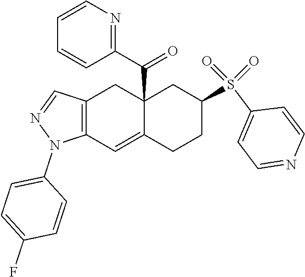 [SMILES: O=C(c1ccccn1)[C@]12Cc3cnn(-c4ccc(F)cc4)c3C=C1CC[C@H](S(=O)(=O)c1ccncc1)C2] | 515.1 |
| 48 | ((4aS,6S)-1-(4-fluorophenyl)-6- (pyridin-4-ylsulfonyl)- 1,4,5,6,7,8-hexahydro-4aH- benzo[f]indazol-4a-yl)(4- methylpyridin-2-yl)methanone | 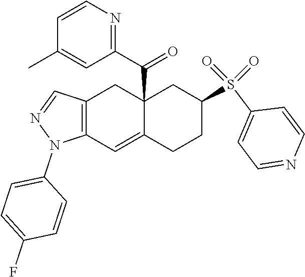 [SMILES: Cc1ccnc(C(=O)[C@]23Cc4cnn(-c5ccc(F)cc5)c4C=C2CC[C@H](S(=O)(=O)c2ccncc2)C3)c1] | 529.3 |
| 49 | ((4aS,6S)-1-(4-fluorophenyl)-6- (neopentylsulfonyl)-1,4,5,6,7,8- hexahydro-4aH- benzo[f]indazol-4a-yl)(pyridin- 2-yl)methanone | 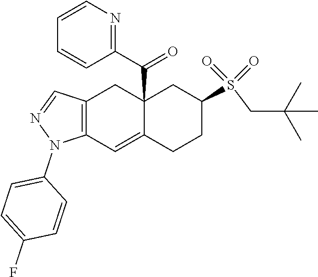 [SMILES: CC(C)(C)CS(=O)(=O)[C@H]1CCC2=Cc3c(cnn3-c3ccc(F)cc3)C[C@]2(C(=O)c2ccccn2)C1] | 508.1 |
| 50 | ((4aS,6S)-6- (cyclopentylsulfonyl)-1-(4- fluorophenyl)-1,4,5,6,7,8- hexahydro-4aH- benzo[f]indazol-4a-yl)(pyridin- 2-yl)methanone | 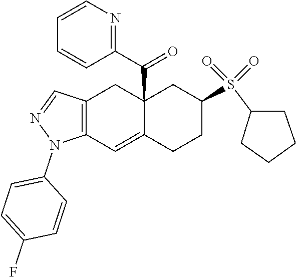 [SMILES: O=C(c1ccccn1)[C@]12Cc3cnn(-c4ccc(F)cc4)c3C=C1CC[C@H](S(=O)(=O)C1CCCC1)C2] | 506.2 |
| 51 | ((4aS,6R)-1-(4-fluorophenyl)-6- (phenylsulfonyl)-1,4,5,6,7,8- hexahydro-4aH- benzo[f]indazol-4a-yl)(5- methylpyridin-2-yl)methanone | 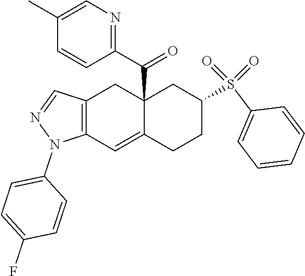 [SMILES: Cc1ccc(C(=O)[C@]23Cc4cnn(-c5ccc(F)cc5)c4C=C2CC[C@@H](S(=O)(=O)c2ccccc2)C3)nc1] | 528.2 |
| 52 | ((4aS,6S)-6-((2-ethyl-2H-1,2,3- triazol-4-yl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 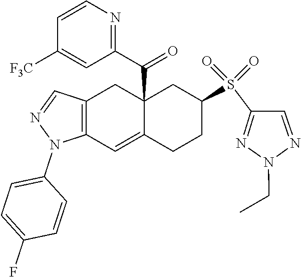 [SMILES: CCn1ncc(S(=O)(=O)[C@H]2CCC3=Cc4c(cnn4-c4ccc(F)cc4)C[C@]3(C(=O)c3cc(C(F)(F)F)ccn3)C2)n1] | 601.2 |
| 53 | ((4aS,6S)-1-(4-fluorophenyl)-6- (pyridin-3-ylsulfonyl)- 1,4,5,6,7,8-hexahydro-4aH- benzo[f]indazol-4a-yl)(pyridin- 2-yl)methanone | 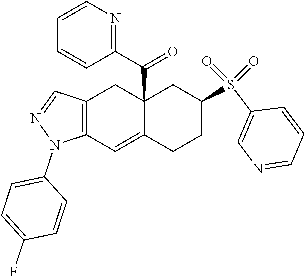 [SMILES: O=C(c1ccccn1)[C@]12Cc3cnn(-c4ccc(F)cc4)c3C=C1CC[C@H](S(=O)(=O)c1cccnc1)C2] | 515.2 |
| 54 | ((4aS,6S)-1-(4-fluorophenyl)-6- (pyridin-3-ylsulfonyl)- 1,4,5,6,7,8-hexahydro-4aH- benzo[f]indazol-4a-yl)(4- (trifluoromethyl)pyridin-2- yl)methanone | 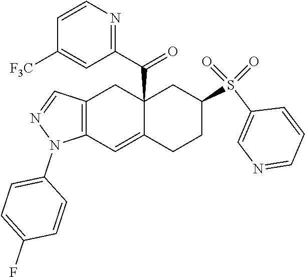 [SMILES: O=C(c1cc(C(F)(F)F)ccn1)[C@]12Cc3cnn(-c4ccc(F)cc4)c3C=C1CC[C@H](S(=O)(=O)c1cccnc1)C2] | 583.2 |
| 55 | ((4aS,6S)-1-(4-fluorophenyl)-6- ((4-fluorophenyl)sulfonyl)- 1,4,5,6,7,8-hexahydro-4aH- benzo[f]indazol-4a-yl)(5- (trifluoromethyl)pyridin-2- yl)methanone | 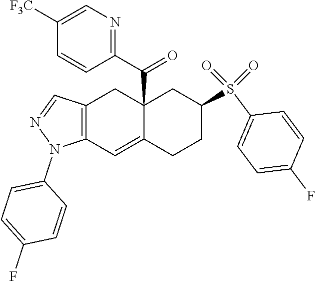 [SMILES: O=C(c1ccc(C(F)(F)F)cn1)[C@]12Cc3cnn(-c4ccc(F)cc4)c3C=C1CC[C@H](S(=O)(=O)c1ccc(F)cc1)C2] | 600.2 |
| 56 | ((4aS,6S)-1-(4-fluorophenyl)-6- ((2-isopropyl-2H-1,2,3-triazol- 4-yl)sulfonyl)-1,4,5,6,7,8- hexahydro-4aH- benzo[f]indazol-4a-yl)(4- (trifluoromethyl)pyridin-2- yl)methanone | 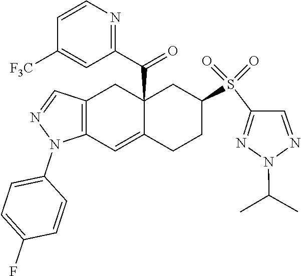 [SMILES: CC(C)n1ncc(S(=O)(=O)[C@H]2CCC3=Cc4c(cnn4-c4ccc(F)cc4)C[C@]3(C(=O)c3cc(C(F)(F)F)ccn3)C2)n1] | 615.2 |
| 57 | (5-cyclopropylpyridin-2- yl)((4aS,6S)-1-(4- fluorophenyl)-6-(pyridin-2- ylsulfonyl)-1,4,5,6,7,8- hexahydro-4aH- benzo[f]indazol-4a- yl)methanone | 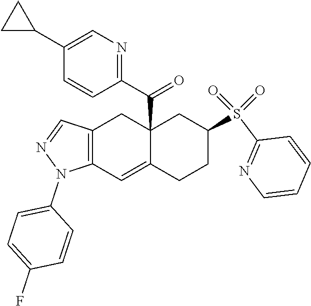 [SMILES: O=C(c1ccc(C2CC2)cn1)[C@]12Cc3cnn(-c4ccc(F)cc4)c3C=C1CC[C@H](S(=O)(=O)c1ccccn1)C2] | 555.2 |
| 58 | ((4aS,6S)-1-(4-fluorophenyl)-6- (pyridin-2-ylsulfonyl)- 1,4,5,6,7,8-hexahydro-4aH- benzo[f]indazol-4a-yl)(5- (trifluoromethyl)pyridin-2- yl)methanone | 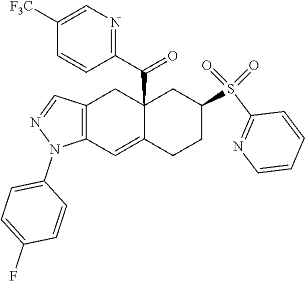 [SMILES: O=C(c1ccc(C(F)(F)F)cn1)[C@]12Cc3cnn(-c4ccc(F)cc4)c3C=C1CC[C@H](S(=O)(=O)c1ccccn1)C2] | 583.1 |
| 59 | ((4aS,6S)-1-(4-fluorophenyl)-6- ((tetrahydro-2H-pyran-4- yl)sulfonyl)-1,4,5,6,7,8- hexahydro-4aH- benzo[f]indazol-4a-yl)(4- (trifluoromethyl)pyridin-2- yl)methanone | 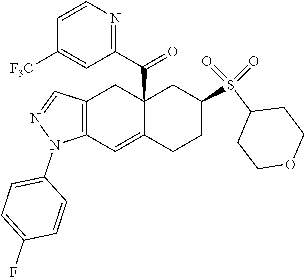 [SMILES: O=C(c1cc(C(F)(F)F)ccn1)[C@]12Cc3cnn(-c4ccc(F)cc4)c3C=C1CC[C@H](S(=O)(=O)C1CCOCC1)C2] | 590.2 |
| 60 | ((4aS,6S)-1-(4-fluorophenyl)-6- ((4-hydroxy-4- methylcyclohexyl)sulfonyl)- 1,4,5,6,7,8-hexahydro-4aH- benzo[f]indazol-4a-yl)(pyridin- 2-yl)methanone | 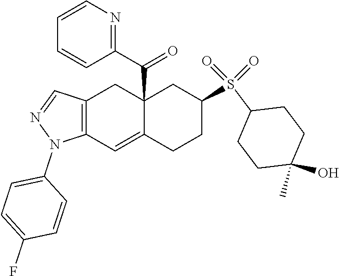 | 550.2 |
| 61 | ((4aS,6S)-1-(4-fluorophenyl)-6- (pyridin-2-ylsulfonyl)- 1,4,5,6,7,8-hexahydro-4aH- benzo[f]indazol-4a-yl)(5- methoxypyridin-2- yl)methanone | 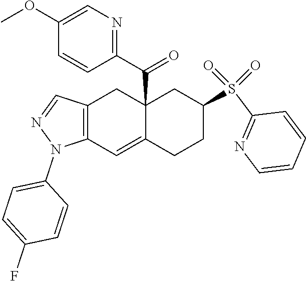 [SMILES: COc1ccc(C(=O)[C@]23Cc4cnn(-c5ccc(F)cc5)c4C=C2CC[C@H](S(=O)(=O)c2ccccn2)C3)nc1] | 545.2 |
| 62 | (5-cyclopropylpyridin-2- yl)((4aS,6S)-1-(4- fluorophenyl)-6-((tetrahydro- 2H-pyran-4-yl)sulfonyl)- 1,4,5,6,7,8-hexahydro-4aH- benzo[f]indazol-4a- yl)methanone | 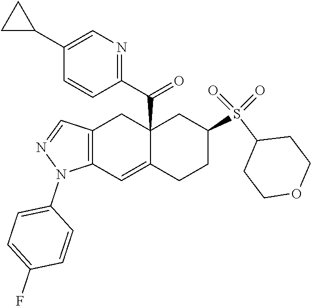 [SMILES: O=C(c1ccc(C2CC2)cn1)[C@]12Cc3cnn(-c4ccc(F)cc4)c3C=C1CC[C@H](S(=O)(=O)C1CCOCC1)C2] | 562.3 |
| 63 | ((4aS,6S)-1-(4-fluorophenyl)-6- (isopropylsulfonyl)-1,4,5,6,7,8- hexahydro-4aH- benzo[f]indazol-4a-yl)(pyridin- 2-yl)methanone | 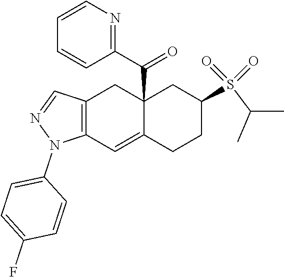 [SMILES: CC(C)S(=O)(=O)[C@H]1CCC2=Cc3c(cnn3-c3ccc(F)cc3)C[C@]2(C(=O)c2ccccn2)C1] | 480.3 |
| 64 | ((4aS,6S)-1-(4-fluorophenyl)-6- ((4-fluorophenyl)sulfonyl)- 1,4,5,6,7,8-hexahydro-4aH- benzo[f]indazol-4a-yl)(4- (trifluoromethyl)pyridin-2- yl)methanone | 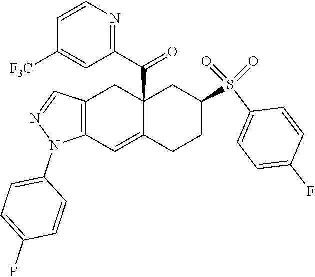 [SMILES: O=C(c1cc(C(F)(F)F)ccn1)[C@]12Cc3cnn(-c4ccc(F)cc4)c3C=C1CC[C@H](S(=O)(=O)c1ccc(F)cc1)C2] | 600.2 |
| 65 | ((4aS,6S)-6-((4,4- difluorocyclohexyl)sulfonyl)-1- (4-fluorophenyl)-1,4,5,6,7,8- hexahydro-4aH- benzo[f]indazol-4a-yl)(pyridin- 2-yl)methanone | 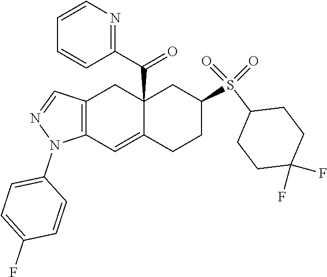 [SMILES: O=C(c1ccccn1)[C@]12Cc3cnn(-c4ccc(F)cc4)c3C=C1CC[C@H](S(=O)(=O)C1CCC(F)(F)CC1)C2] | 556.2 |
| 66 | ((4aS,6S)-1-(4-fluorophenyl)-6- ((1-methyl-1H-pyrazol-4- yl)sulfonyl)-1,4,5,6,7,8- hexahydro-4aH- benzo[f]indazol-4a-yl)(5- (trifluoromethoxy)pyridin-2- yl)methanone | 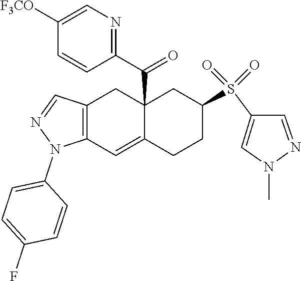 [SMILES: Cn1cc(S(=O)(=O)[C@H]2CCC3=Cc4c(cnn4-c4ccc(F)cc4)C[C@]3(C(=O)c3ccc(OC(F)(F)F)cn3)C2)cn1] | 602.2 |
| 67 | ((4aS,6S)-6- (cyclobutylsulfonyl)-1-(4- fluorophenyl)-1,4,5,6,7,8- hexahydro-4aH- benzo[f]indazol-4a-yl)(pyridin- 2-yl)methanone | 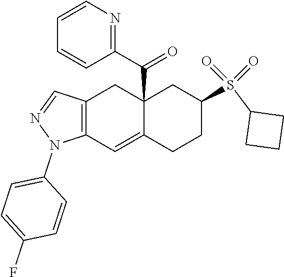 [SMILES: O=C(c1ccccn1)[C@]12Cc3cnn(-c4ccc(F)cc4)c3C=C1CC[C@H](S(=O)(=O)C1CCC1)C2] | 492.1 |
| 68 | ((4aS,6S)-1-(4-fluorophenyl)-6- (pyridin-2-ylsulfonyl)- 1,4,5,6,7,8-hexahydro-4aH- benzo[f]indazol-4a-yl)(5- fluoropyridin-2-yl)methanone | 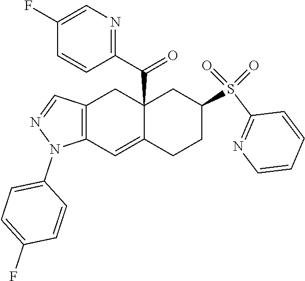 [SMILES: O=C(c1ccc(F)cn1)[C@]12Cc3cnn(-c4ccc(F)cc4)c3C=C1CC[C@H](S(=O)(=O)c1ccccn1)C2] | 533.2 |
| 69 | ((4aS,6S)-1-(4-fluorophenyl)-6- ((1-methyl-1H-pyrazol-4- yl)sulfonyl)-1,4,5,6,7,8- hexahydro-4aH- benzo[f]indazol-4a-yl)(5- (trifluoromethyl)pyridin-2- yl)methanone | 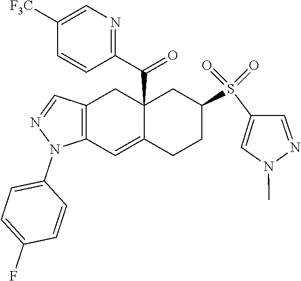 [SMILES: Cn1cc(S(=O)(=O)[C@H]2CCC3=Cc4c(cnn4-c4ccc(F)cc4)C[C@]3(C(=O)c3ccc(C(F)(F)F)cn3)C2)cn1] | 586.2 |
| 70 | ((4aS,6S)-1-(4-fluorophenyl)-6- ((tetrahydro-2H-pyran-4- yl)sulfonyl)-1,4,5,6,7,8- hexahydro-4aH- benzo[f]indazol-4a-yl)(5- (trifluoromethyl)pyridin-2- yl)methanone | 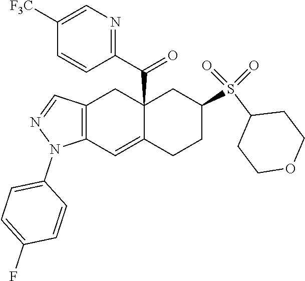 [SMILES: O=C(c1ccc(C(F)(F)F)cn1)[C@]12Cc3cnn(-c4ccc(F)cc4)c3C=C1CC[C@H](S(=O)(=O)C1CCOCC1)C2] | 590.2 |
| 71 | ((4aS,6S)-6-((1-cyclopropyl- 1H-pyrazol-4-yl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 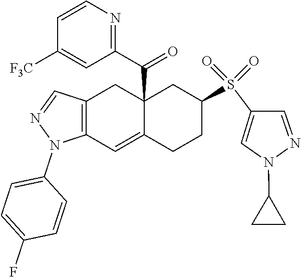 [SMILES: O=C(c1cc(C(F)(F)F)ccn1)[C@]12Cc3cnn(-c4ccc(F)cc4)c3C=C1CC[C@H](S(=O)(=O)c1cnn(C3CC3)c1)C2] | 612.2 |
| 72 | (5-cyclopropylpyridin-2- yl)((4aS,6S)-1-(4- fluorophenyl)-6-((1-methyl-1H- pyrazol-4-yl)sulfonyl)- 1,4,5,6,7,8-hexahydro-4aH- benzo[f]indazol-4a- yl)methanone | 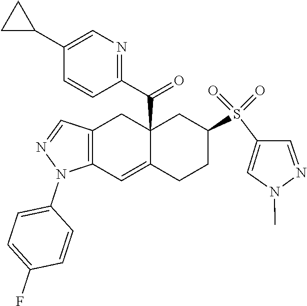 [SMILES: Cn1cc(S(=O)(=O)[C@H]2CCC3=Cc4c(cnn4-c4ccc(F)cc4)C[C@]3(C(=O)c3ccc(C4CC4)cn3)C2)cn1] | 558.2 |
| 73 | ((4aS,6S)-6-((2-cyclopropyl- 2H-1,2,3-triazol-4-yl)sulfonyl)- 1-(4-fluorophenyl)-1,4,5,6,7,8- hexahydro-4aH- benzo[f]indazol-4a-yl)(4- (trifluoromethyl)pyridin-2- yl)methanone | 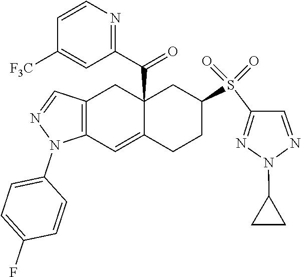 [SMILES: O=C(c1cc(C(F)(F)F)ccn1)[C@]12Cc3cnn(-c4ccc(F)cc4)c3C=C1CC[C@H](S(=O)(=O)c1cnn(C3CC3)n1)C2] | 613.2 |
| 74 | (5-chloropyridin-2- yl)((4aS,6S)-1-(4- fluorophenyl)-6-(pyridin-2- ylsulfonyl)-1,4,5,6,7,8- hexahydro-4aH- benzo[f]indazol-4a- yl)methanone | 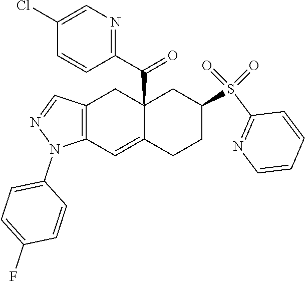 [SMILES: O=C(c1ccc(Cl)cn1)[C@]12Cc3cnn(-c4ccc(F)cc4)c3C=C1CC[C@H](S(=O)(=O)c1ccccn1)C2] | 549.2 |
| 75 | ((4aS,6S)-1-(4-fluorophenyl)-6- ((1-(2,2,2-trifluoroethyl)-1H- pyrazol-4-yl)sulfonyl)- 1,4,5,6,7,8-hexahydro-4aH- benzo[f]indazol-4a-yl)(4- (trifluoromethyl)pyridin-2- yl)methanone | 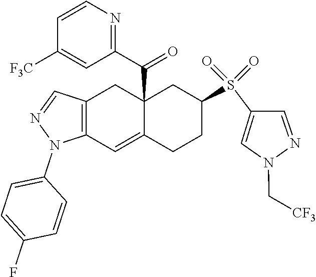 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)c1)C2] | 654.1 |
| 76 | ((4aS,6S)-6-((1-ethyl-1H- pyrazol-4-yl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 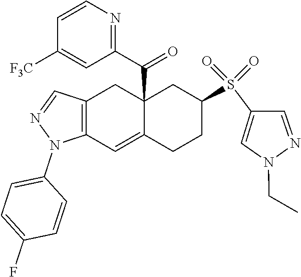 [SMILES: CCn1cc(S(=O)(=O)[C@H]2CCC3=Cc4c(cnn4-c4ccc(F)cc4)C[C@]3(C(=O)c3cc(C(F)(F)F)ccn3)C2)cn1] | 615.2 |
| 77 | ((4aS,6S)-6-((2-ethyl-2H-1,2,3- triazol-4-yl)sulfonyl)-1-(4- fluorophenyl)-1,4,5,6,7,8- hexahydro-4aH- benzo[f]indazol-4a-yl)(5- (trifluoromethyl)pyridin-2- yl)methanone | 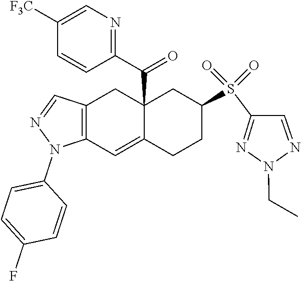 [SMILES: CCn1ncc(S(=O)(=O)[C@H]2CCC3=Cc4c(cnn4-c4ccc(F)cc4)C[C@]3(C(=O)c3ccc(C(F)(F)F)cn3)C2)n1] | 601.2 |
| 78 | N-((4aS,6S)-4a-(5- cyclopropylpicolinoyl)-1-(4- fluorophenyl)-4,4a,5,6,7,8- hexahydro-1H-benzo[f]indazol- 6-yl)-N,2-dimethyl-2H-1,2,3- triazole-4-sulfonamide | 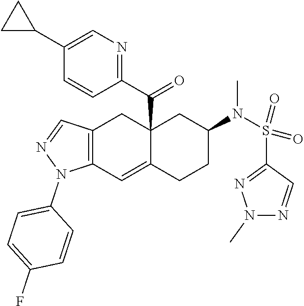 [SMILES: CN([C@H]1CCC2=Cc3c(cnn3-c3ccc(F)cc3)C[C@]2(C(=O)c2ccc(C3CC3)cn2)C1)S(=O)(=O)c1cnn(C)n1] | 589.2 |
| 79 | ((4aS,6S)-6-((2- (cyclopropylmethyl)-2H-1,2,3- triazol-4-yl)sulfonyl)-1-(4- fluorophenyl)-1,4,5,6,7,8- hexahydro-4aH- benzo[f]indazol-4a-yl)(4- (trifluoromethyl)pyridin-2- yl)methanone | 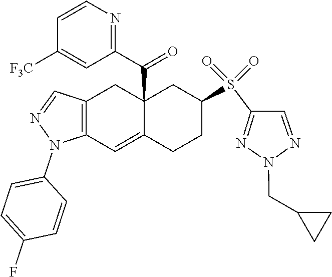 [SMILES: O=C(c1cc(C(F)(F)F)ccn1)[C@]12Cc3cnn(-c4ccc(F)cc4)c3C=C1CC[C@H](S(=O)(=O)c1cnn(CC3CC3)n1)C2] | 627.2 |
| 80 | N-((4aS,6S)-1-(4- fluorophenyl)-4a-picolinoyl- 4,4a,5,6,7,8-hexahydro-1H- benzo[f]indazol-6-yl)-N- methylbenzenesulfonamide | 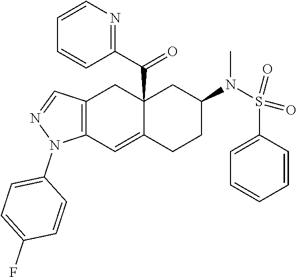 [SMILES: CN([C@H]1CCC2=Cc3c(cnn3-c3ccc(F)cc3)C[C@]2(C(=O)c2ccccn2)C1)S(=O)(=O)c1ccccc1] | 543.2 |
| 81 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-4- sulfonamide | 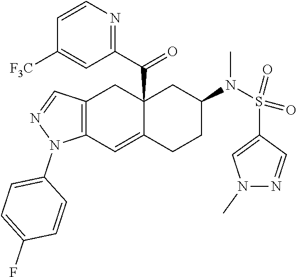 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)c1] | 615.2 |
| 82 | 3-chloro-N-((4aS,6S)-1-(4- fluorophenyl)-4a-picolinoyl- 4,4a,5,6,7,8-hexahydro-1H- benzo[f]indazol-6-yl)-N- methylbenzenesulfonamide | 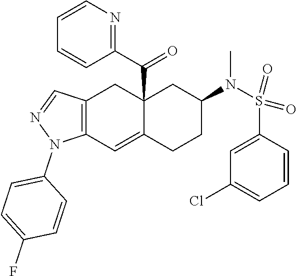 [SMILES: CN([C@H]1CCC2=Cc3c(cnn3-c3ccc(F)cc3)C[C@]2(C(=O)c2ccccn2)C1)S(=O)(=O)c1cccc(Cl)c1] | 577.1 |
| 83 | N-((4aS,6S)-1-(4- fluorophenyl)-4a-picolinoyl- 4,4a,5,6,7,8-hexahydro-1H- benzo[f]indazol-6-yl)-N- methyl-3- (trifluoromethyl)benzenesulfon- amide | 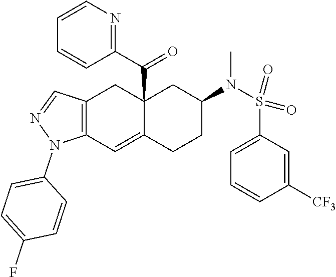 [SMILES: CN([C@H]1CCC2=Cc3c(cnn3-c3ccc(F)cc3)C[C@]2(C(=O)c2ccccn2)C1)S(=O)(=O)c1cccc(C(F)(F)F)c1] | 611.2 |
| 84 | N-((4aS,6S)-1-(4- fluorophenyl)-4a-picolinoyl- 4,4a,5,6,7,8-hexahydro-1H- benzo[f]indazol-6-yl)-N- methylpyridine-3-sulfonamide | 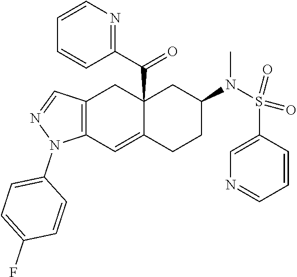 [SMILES: CN([C@H]1CCC2=Cc3c(cnn3-c3ccc(F)cc3)C[C@]2(C(=O)c2ccccn2)C1)S(=O)(=O)c1cccnc1] | 544.1 |
| 85 | N-((4aS,6S)-1-(4- fluorophenyl)-4a-picolinoyl- 4,4a,5,6,7,8-hexahydro-1H- benzo[f]indazol-6-yl)-N- methyl-4- (trifluoromethyl)benzenesulfon- amide | 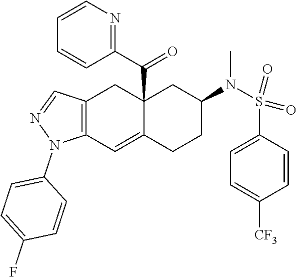 [SMILES: CN([C@H]1CCC2=Cc3c(cnn3-c3ccc(F)cc3)C[C@]2(C(=O)c2ccccn2)C1)S(=O)(=O)c1ccc(C(F)(F)F)cc1] | 611.2 |
| 86 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methyl-4- (trifluoromethyl)benzenesulfon- amide | 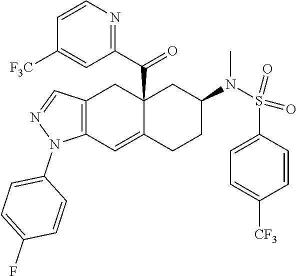 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccc(C(F)(F)F)cc1] | 679.1 |
| 87 | 1-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methyl-1H-pyrazole-4- sulfonamide | 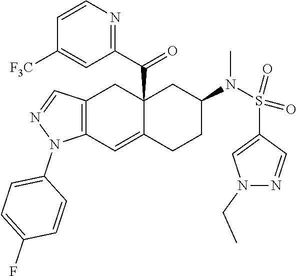 [SMILES: CCn1cc(S(=O)(=O)N(C)[C@H]2CCC3=Cc4c(cnn4-c4ccc(F)cc4)C[C@]3(C(=O)c3cc(C(F)(F)F)ccn3)C2)cn1] | 629.2 |
| 88 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1-methyl- 1H-pyrazole-4-sulfonamide | 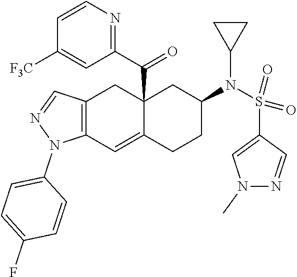 [SMILES: Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1] | 641.2 |
| 89 | N-((4aS,6S)-1-(4- fluorophenyl)-4a-picolinoyl- 4,4a,5,6,7,8-hexahydro-1H- benzo[f]indazol-6-yl)-N- methylbenzamide | 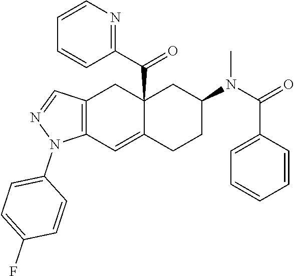 [SMILES: CN(C(=O)c1ccccc1)[C@H]1CCC2=Cc3c(cnn3-c3ccc(F)cc3)C[C@]2(C(=O)c2ccccn2)C1] | 507.2 |
| 90 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,2- dimethyl-2H-1,2,3-triazole-4- sulfonamide | 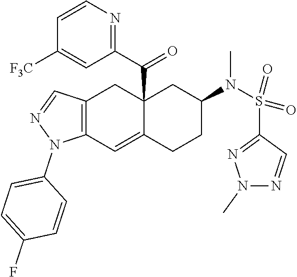 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)n1] | 616.3 |
| 91 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1-methyl- 1H-pyrazole-4-sulfonamide | 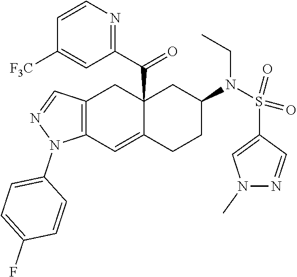 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cnn(C)c1] | 629.2 |
| 92 | (4aS,6S)-1-(4-fluorophenyl)-N- methyl-N-phenyl-4a-picolinoyl- 4,4a,5,6,7,8-hexahydro-1H- benzo[f]indazole-6-sulfonamide | 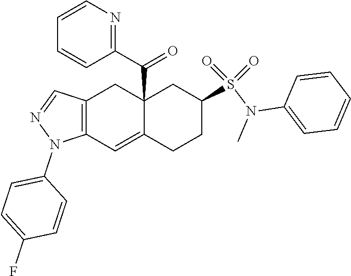 [SMILES: CN(c1ccccc1)S(=O)(=O)[C@H]1CCC2=Cc3c(cnn3-c3ccc(F)cc3)C[C@]2(C(=O)c2ccccn2)C1] | 543.2 |
| 93 | N-((4aS,6S)-4a-(5- cyclopropylpicolinoyl)-1-(4- fluorophenyl)-4,4a,5,6,7,8- hexahydro-1H-benzo[f]indazol- 6-yl)-N,1-dimethyl-1H- pyrazole-4-sulfonamide | 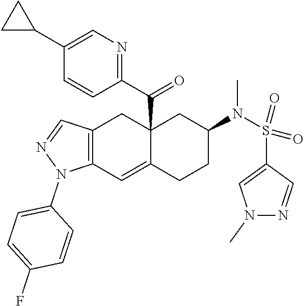 [SMILES: CN([C@H]1CCC2=Cc3c(cnn3-c3ccc(F)cc3)C[C@]2(C(=O)c2ccc(C3CC3)cn2)C1)S(=O)(=O)c1cnn(C)c1] | 587.2 |
| 94 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methyltetrahydro-2H-pyran-4- sulfonamide | 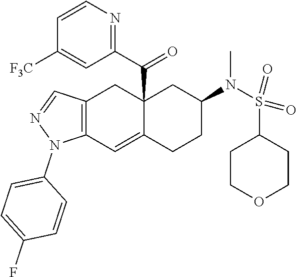 [SMILES: CN([C@H]1CCC2=Cc3c(cnn3-c3ccc(F)cc3)C[C@]2(C(=O)c2cc(C(F)(F)F)ccn2)C1)S(=O)(=O)C1CCOCC1] | 619.2 |
| 95 | N-((4aS,6R)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-4- sulfonamide | 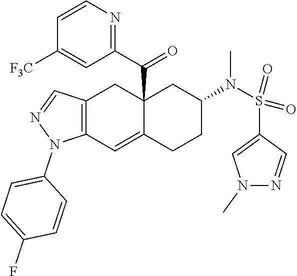 [SMILES: CN([C@@H]1CCC2=Cc3c(cnn3-c3ccc(F)cc3)C[C@]2(C(=O)c2cc(C(F)(F)F)ccn2)C1)S(=O)(=O)c1cnn(C)c1] | 615.2 |
| 96 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-4- carboxamide | 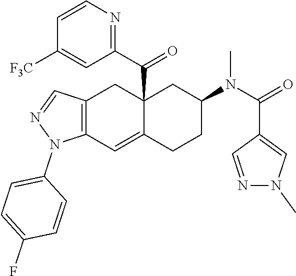 [SMILES: CN(C(=O)c1cnn(C)c1)[C@H]1CCC2=Cc3c(cnn3-c3ccc(F)cc3)C[C@]2(C(=O)c2cc(C(F)(F)F)ccn2)C1] | 579.2 |
| 97 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- isopropyl-N-methyl-1H- pyrazole-4-sulfonamide | 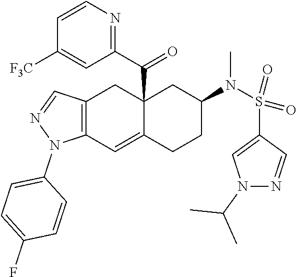 [SMILES: CC(C)n1cc(S(=O)(=O)N(C)[C@H]2CCC3=Cc4c(cnn4-c4ccc(F)cc4)C[C@]3(C(=O)c3cc(C(F)(F)F)ccn3)C2)cn1] | 643.2 |
| 98 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-3- sulfonamide | 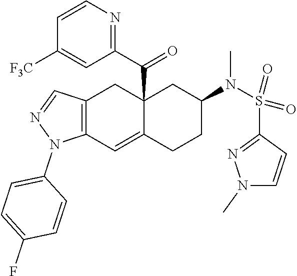 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccn(C)n1] | 615.1 |
| 99 | 4-chloro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methylbenzenesulfonamide | 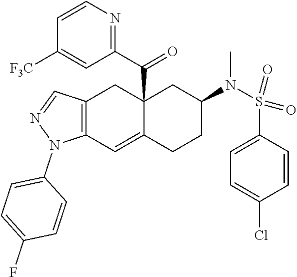 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccc(Cl)cc1] | 645.0 |
| 100 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-4- sulfonamide | 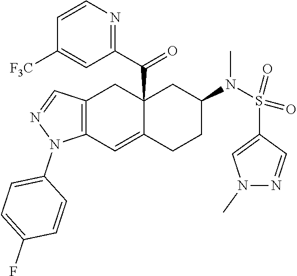 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)c1] | 617.2 |
| 101 | (4aS,6S)-1-(4-fluorophenyl)-N- methyl-N-(1-methyl-1H- pyrazol-4-yl)-4a-(4- (trifluoromethyl)picolinoyl)- 4,4a,5,6,7,8-hexahydro-1H- benzo[f]indazole-6-sulfonamide | 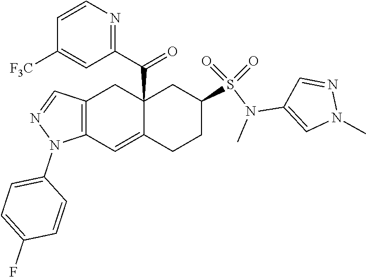 [SMILES: CN(c1cnn(C)c1)S(=O)(=O)[C@H]1CCC2=Cc3c(cnn3-c3ccc(F)cc3)C[C@]2(C(=O)c2cc(C(F)(F)F)ccn2)C1] | 615.2 |
| 102 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1-methyl- 1H-pyrazole-4-carboxamide | 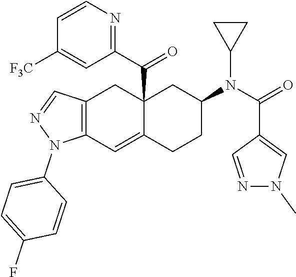 [SMILES: Cn1cc(C(=O)N(C2CC2)[C@H]2CCC3=Cc4c(cnn4-c4ccc(F)cc4)C[C@]3(C(=O)c3cc(C(F)(F)F)ccn3)C2)cn1] | 605.3 |
| 103 | N-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1-methyl- 1H-pyrazole-4-sulfonamide | 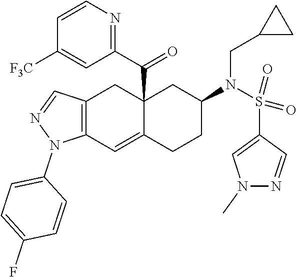 [SMILES: Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)cn1] | 655.2 |
| 104 | N-((4aS,6S)-1-(4- fluorophenyl)-4a-(5- (trifluoromethyl)picolinoyl)- 4,4a,5,6,7,8-hexahydro-1H- benzo[f]indazol-6-yl)-N,2- dimethyl-2H-1,2,3-triazole-4- sulfonamide | 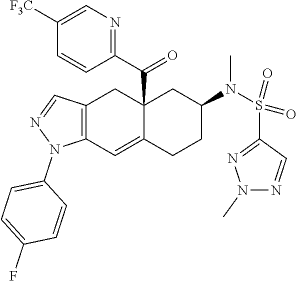 [SMILES: CN([C@H]1CCC2=Cc3c(cnn3-c3ccc(F)cc3)C[C@]2(C(=O)c2ccc(C(F)(F)F)cn2)C1)S(=O)(=O)c1cnn(C)n1] | 616.3 |
| 105 | 4-chloro-N-((4aS,6S)-1-(4- fluorophenyl)-4a-(5- (trifluoromethyl)picolinoyl)- 4,4a,5,6,7,8-hexahydro-1H- benzo[f]indazol-6-yl)-N- methylbenzenesulfonamide | 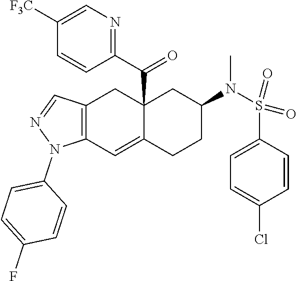 [SMILES: CN([C@H]1CCC2=Cc3c(cnn3-c3ccc(F)cc3)C[C@]2(C(=O)c2ccc(C(F)(F)F)cn2)C1)S(=O)(=O)c1ccc(Cl)cc1] | 645.0 |
| 106 | 4-fluoro-N-((4aS,6S)-1-(4- fluorophenyl)-4a-(thiazole-2- carbonyl)-4,4a,5,6,7,8- hexahydro-1H-benzo[f]indazol- 6-yl)-N- methylbenzenesulfonamide | 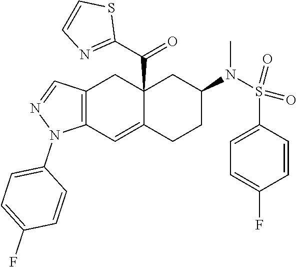 [SMILES: CN([C@H]1CCC2=Cc3c(cnn3-c3ccc(F)cc3)C[C@]2(C(=O)c2nccs2)C1)S(=O)(=O)c1ccc(F)cc1] | 567.2 |
| 107 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,2- dimethyl-2H-1,2,3-triazole-4- sulfonamide | 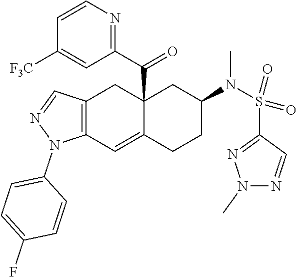 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)n1] | 616.3 |
| 108 | 1-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-N- methyl-1H-pyrazole-4- sulfonamide | 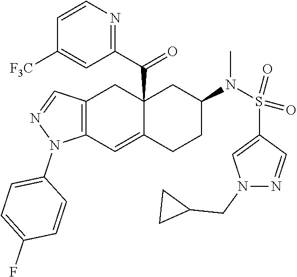 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(CC2CC2)c1] | 655.3 |
| 109 | N-((4aS,6S)-4a-(5- cyclopropylpicolinoyl)-1-(4- fluorophenyl)-4,4a,5,6,7,8- hexahydro-1H-benzo[f]indazol- 6-yl)-N-ethyl-4- fluorobenzenesulfonamide | 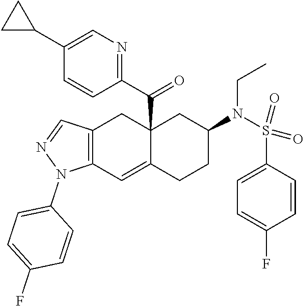 [SMILES: CCN([C@H]1CCC2=Cc3c(cnn3-c3ccc(F)cc3)C[C@]2(C(=O)c2ccc(C3CC3)cn2)C1)S(=O)(=O)c1ccc(F)cc1] | 615.2 |
| 110 | 4-chloro-N-((4aS,6S)-4a-(5- cyclopropylpicolinoyl)-1-(4- fluorophenyl)-4,4a,5,6,7,8- hexahydro-1H-benzo[f]indazol- 6-yl)-N- ethylbenzenesulfonamide | 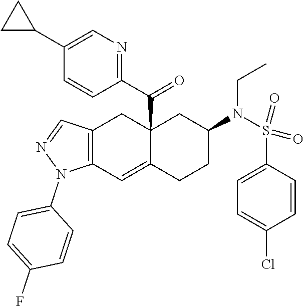 [SMILES: CCN([C@H]1CCC2=Cc3c(cnn3-c3ccc(F)cc3)C[C@]2(C(=O)c2ccc(C3CC3)cn2)C1)S(=O)(=O)c1ccc(Cl)cc1] | 631.2 |

### Example 111. N-((4aS,6S)-1-(4-Fluorophenyl)-4a-(4-(trifluoromethyl)picolinoyl)-4,4a,5,6,7,8-hexahydro-1H-benzo[f]indazol-6-yl)-1-methyl-N-(2,2,2-trifluoroethyl)-1H-pyrazole-3-sulfonamide (111)

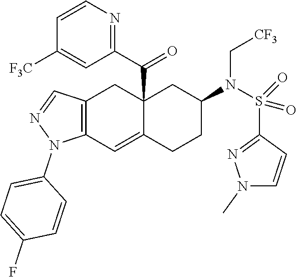 [SMILES: Cn1ccc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1]

### Step A: Ethyl(4aS,6S)-6-((2,4-dimethoxybenzyl)amino)-1-(4-fluorophenyl)-1,4,5,6,7,8-hexahydro-4aH-benzo[f]indazole-4a-carboxylate (111a)

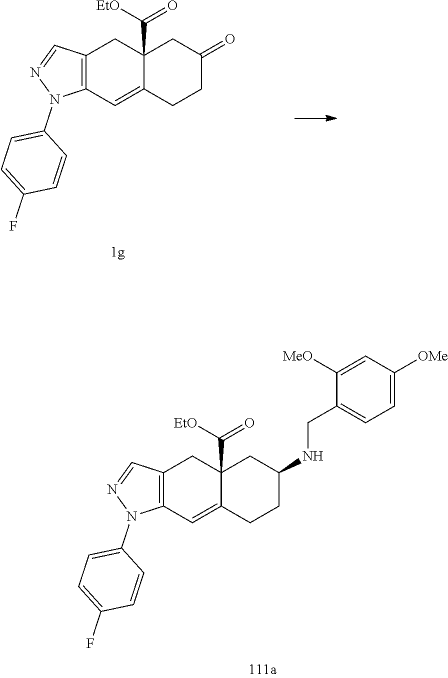 [SMILES: CCOC(=O)[C@@]12CC(=O)CCC1=Cc1c(cnn1-c1ccc(F)cc1)C2 ; CCOC(=O)[C@]12Cc3cnn(-c4ccc(F)cc4)c3C=C1CC[C@H](NCc1ccc(OC)cc1OC)C2]

To a solution of ethyl (4aS)-1-(4-fluorophenyl)-6-oxo-4,5,7,8-tetrahydrobenzo[f]indazole-4a-carboxylate (1 g) (3.16 g, 8.92 mmol) and (2,4-dimethoxyphenyl)methanamine (2.68 mL, 17.83 mmol) in DCE (40 mL) was added acetic acid (1.54 mL, 26.75 mmol). After the reaction was stirred at rt for 5 min and cooled down in an ice-bath, sodium triacetoxyborohydride (5.1 g, 24.08 mmol) was added in portions. 5 Minute later, the reaction solution was allowed to warm to rt and continued stirring for 30 min. The solution was quenched (sat. aq. NaHCO₃) and extracted (EtOAc). The organic layers were washed (brine), dried (Na₂SO₄) and concentrated under reduced pressure. The crude product was purified by silica gel chromatography (SiO₂, 0%-75% EtOAc/hexanes, gradient elution) to provide the title compound (111a) (4.15 g, 92% yield) as an off-white solid. m/z (ESI, +ve ion)=506.3 [M+H]⁺.

### Step B: Ethyl (4aS,6S)-6-((N-(2,4-dimethoxybenzyl)-1-methyl-1H-pyrazole)-3-sulfonamido)-1-(4-fluorophenyl)-1,4,5,6,7,8-hexahydro-4aH-benzo[f]indazole-4a-carboxylate (111b)

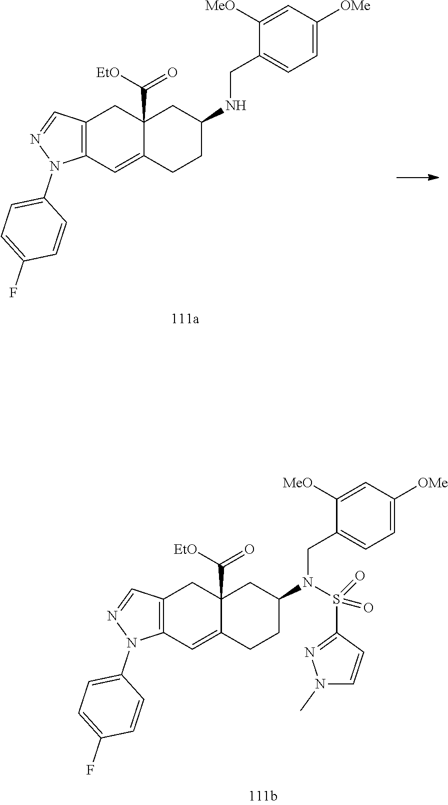 [SMILES: CCOC(=O)[C@]12Cc3cnn(-c4ccc(F)cc4)c3C=C1CC[C@H](NCc1ccc(OC)cc1OC)C2 ; CCOC(=O)[C@]12Cc3cnn(-c4ccc(F)cc4)c3C=C1CC[C@H](N(Cc1ccc(OC)cc1OC)S(=O)(=O)c1ccn(C)n1)C2]

To a 50 mL flask was added 1-methyl-H-pyrazole-3-sulfonyl chloride (1.12 g, 6.22 mmol), ethyl (4aS,6S)-6-[(2,4-dimethoxyphenyl)methylamino]-1-(4-fluorophenyl)-5,6,7,8-tetrahydro-4H-benzo[f]indazole-4a-carboxylate (111a) (1.57 g, 3.11 mmol), DCM (8 mL), and triethylamine (1.73 mL, 12.45 mmol) successively. After the reaction was stirred at 40° C. for 5 h, it was directly concentrated under reduced pressure. The crude product was purified by silica gel column chromatography (SiO₂, 40%-65% EtOAc/hexanes, gradient elution) to provide ethyl (4aS,6S)-6-[(2,4-dimethoxyphenyl)methyl-(1-methylpyrazol-3-yl)sulfonyl-amino]-1-(4-fluorophenyl)-5,6,7,8-tetrahydro-4H-benzo[f]indazole-4a-carboxylate (111b) (1.86 g, 92% yield) as a yellow solid. m/z (ESI, +ve ion)=650.3 [M+H]⁺.

### Step C: N-(2,4-Dimethoxybenzyl)-N-((4aS,6S)-1-(4-fluorophenyl)-4a-(4-(trifluoromethyl)picolinoyl)-4,4a,5,6,7,8-hexahydro-1H-benzo[f]indazol-6-yl)-1-methyl-1H-pyrazole-3-sulfonamide (111c)

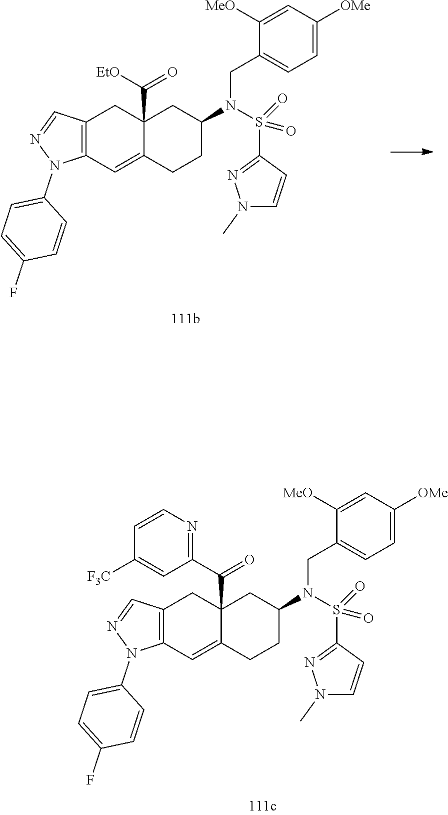 [SMILES: CCOC(=O)[C@]12Cc3cnn(-c4ccc(F)cc4)c3C=C1CC[C@H](N(Cc1ccc(OC)cc1OC)S(=O)(=O)c1ccn(C)n1)C2 ; COc1ccc(CN([C@H]2CCC3=Cc4c(cnn4-c4ccc(F)cc4)C[C@]3(C(=O)c3cc(C(F)(F)F)ccn3)C2)S(=O)(=O)c2ccn(C)n2)c(OC)c1]

To a 100 mL dried flask charged with anhydrous ether (17 mL) was added n-butyllithium (5.37 mL, 8.59 mmol), followed by dropwise addition of 2-bromo-4-(trifluoromethyl)pyridine (1.17 mL, 9.45 mmol) at −78° C. The reaction was stirred for another 15 min and a solution of ethyl (4aS,6S)-6-[(2,4-dimethoxyphenyl)methyl-(1-methylpyrazol-3-yl)sulfonyl-amino]-1-(4-fluorophenyl)-5,6,7,8-tetrahydro-4H-benzo[f]indazole-4a-carboxylate (IIb) (1.86 g, 2.86 mmol) in ether (9 mL) and THF (2.0 mL) was added dropwise. After the reaction was stirred at −78° C. for 25 min, it was quenched with a small amount of water and then sat. NH₄Cl, extracted (EtOAc), washed (sat. aq. NaCl,) and dried (Na₂SO₄). The combined organic layers were concentrated under reduced pressure and the residue was dissolved in acetonitrile (6 mL) and 1 N HCl (6 mL). The solution was stirred for 1.5 h, diluted (EtOAc), quenched (sat. NaHCO₃), extracted (EtOAc), washed (sat. aq. NaCl), and dried (Na₂SO₄). The combined organic layers were concentrated under reduced pressure. The crude product was purified by silica gel column chromatography (SiO₂, 40%-65% EtOAc/hexanes, gradient elution) to provide the title compound (111c) (1.93 g, 89.8% yield) as a yellow solid. m/z (ESI, +ve ion)=751.2 [M+H]⁺.

### Step D: N-((4aS,6S)-1-(4-Fluorophenyl)-4a-(4-(trifluoromethyl)picolinoyl)-4,4a,5,6,7,8-hexahydro-1H-benzo[f]indazol-6-yl)-1-methyl-1H-pyrazole-3-sulfonamide (111d)

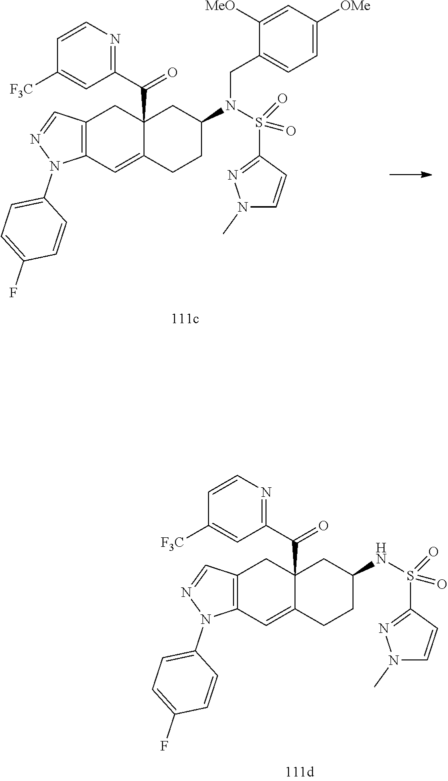 [SMILES: COc1ccc(CN([C@H]2CCC3=Cc4c(cnn4-c4ccc(F)cc4)C[C@]3(C(=O)c3cc(C(F)(F)F)ccn3)C2)S(=O)(=O)c2ccn(C)n2)c(OC)c1 ; Cn1ccc(S(=O)(=O)N[C@H]2CCC3=Cc4c(cnn4-c4ccc(F)cc4)C[C@]3(C(=O)c3cc(C(F)(F)F)ccn3)C2)n1]

To a pressure vial charged with N-[(4aS,6S)-1-(4-fluorophenyl)-4a-[4-(trifluoromethyl)pyridine-2-carbonyl]-5,6,7,8-tetrahydro-4H-benzo[f]indazol-6-yl]-N-[(2,4-dimethoxyphenyl)methyl]-1-methyl-pyrazole-3-sulfonamide (111c) (107 mg, 0.14 mmol) was added TFA (0.3 mL) and DCM (0.7 mL). The reaction was stirred at rt for 1 h and then concentrated under reduced pressure. The residue was azeotroped with toluene and directly purified by silica gel column chromatography (SiO₂, 0%-75% EtOAc/hexanes, gradient elution) to provide the title compound (111d) (60 mg, 70% yield) as a white solid. m/z (ESI, +ve ion)=601.2 [M+H]⁺.

### Step E: N-((4aS,6S)-1-(4-Fluorophenyl)-4a-(4-(trifluoromethyl)picolinoyl)-4,4a,5,6,7,8-hexahydro-1H-benzo[f]indazol-6-yl)-1-methyl-N-(2,2,2-trifluoroethyl)-1H-pyrazole-3-sulfonamide (111)

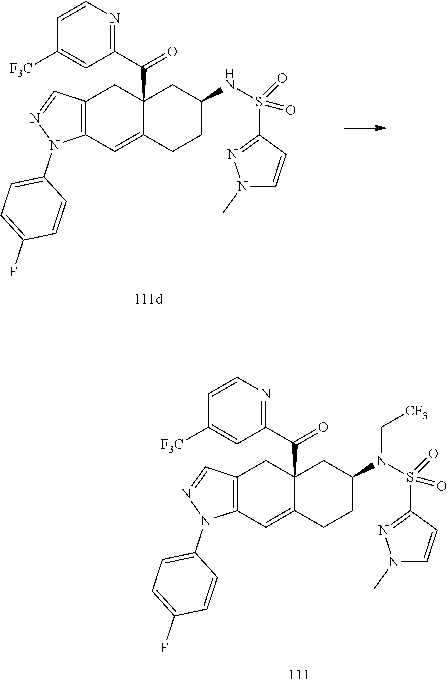 [SMILES: Cn1ccc(S(=O)(=O)N[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1]

A flask was charged with N-[(4aS,6S)-1-(4-fluorophenyl)-4a-[4-(trifluoromethyl)pyridine-2-carbonyl]-5,6,7,8-tetrahydro-4H-benzo[f]indazol-6-yl]-1-methyl-pyrazole-3-sulfonamide (111d) (1.89 g, 3.15 mmol) and sodium hydride (377 mg, 9.44 mmol). After the flask was put under high vacuum for 20 min and flushed with argon, DMF (32 mL) was added to form a homogeneous solution. The reaction was cooled down in an ice-bath and 2,2,2-trifluoroethyl triflate (2.27 mL, 15.73 mmol) was slowly added. 5 Minute later, the reaction was allowed to warm up to rt and continued stirring for 3 h. The reaction was cooled back down in an ice-bath, quenched with 10% citric acid and extracted with EtOAc. The organic layer was washed (brine), dried, and concentrated. The residue was purified by silica gel column chromatography (SiO₂, 0%-55% EtOAc/hexanes, gradient elution). The crude product was purified by reverse HPLC (50%-70% water/acetonitrile with 0.1% formic acid) to provide the title compound (111) (1.01 g, 47% yield) as a yellow solid. ¹H NMR (400 MHz, Chloroform-d) δ ppm 8.85 (d, J=5.12 Hz, 1H), 8.05 (m, 1H), 7.68 (d, J=5.04 Hz, 1H), 7.39-7.49 (m, 3H), 7.26 (s, 1H), 7.16 (t, J=8.07 Hz, 2H), 6.69 (d, J=2.34 Hz, 1H), 6.46 (d, J=1.75 Hz, 1H), 4.23-4.34 (m, 1H), 4.00 (s, 3H), 3.90-3.97 (m, 2H), 3.86 (d, J=16.52 Hz, 1H), 3.08 (d, J=16.66 Hz, 1H), 2.57-2.69 (m, 2H), 2.34-2.52 (m, 2H), 1.78-1.98 (m, 2H); m/z (ESI, +ve ion)=683.2 [M+H]⁺.

### Example 112. N-(2-Fluoroethyl)-N-((4aS,6S)-1-(4-fluorophenyl)-4a-(5-(trifluoromethyl)thiazole-2-carbonyl)-4,4a,5,6,7,8-hexahydro-1H-benzo[f]indazol-6-yl)-1-methyl-1H-1,2,4-triazole-3-sulfonamide (112)

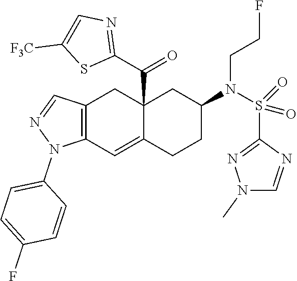 [SMILES: Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1]

### Step A: Ethyl(4aS,6S)-1-(4-fluorophenyl)-6-((1-methyl-1H-1,2,4-triazole)-3-sulfonamido)-1,4,5,6,7,8-hexahydro-4aH-benzo[f]indazole-4a-carboxylate (112a)

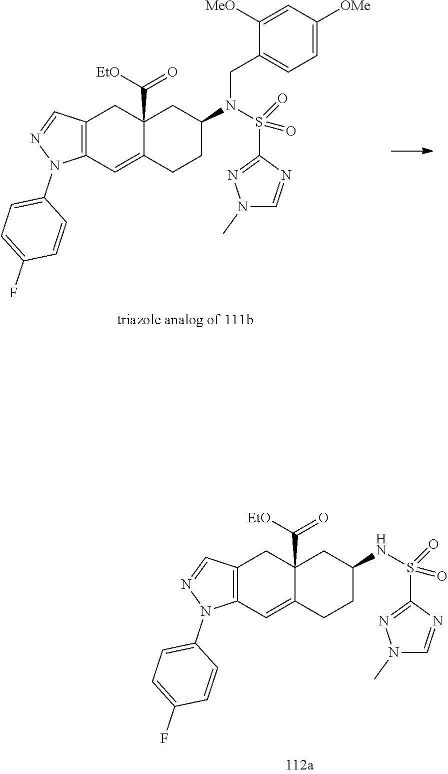

Ethyl (4aS,6S)-6-[(2,4-dimethoxyphenyl)methyl-[(1-methyl-1,2,4-triazol-3-yl)sulfonyl]amino]-1-(4-fluorophenyl)-5,6,7,8-tetrahydro-4H-benzo[f]indazole-4a-carboxylate (triazole analog of 111b) (397 mg, 0.61 mmol) was dissolved in a mixture of trifluoroacetic acid (3.06 mL, 39.66 mmol) and DCM (7 mL). The reaction was stirred for 1 h and additional TFA (0.1 mL) was added. After the reaction was stirred for another 1.5 h, it was poured into an ice-cold NaHCO₃ solution and extracted (EtOAc). The organic phase was filtered through a pad of celite, washed (brine), dried (Na₂SO₄) and concentrated. The crude product was purified by silica gel chromatography (SiO₂, 0%-10% MeOH/DCM, gradient elution) to provide the title compound (112a) (306 mg, 100% yield) as a white solid. m/z (ESI, +ve ion)=501.3 [M+H]⁺.

### Step B: Ethyl (4aS,6S)-6-((N-(2-fluoroethyl)-1-methyl-1H-1,2,4-triazole)-3-sulfonamido)-1-(4-fluorophenyl)-1,4,5,6,7,8-hexahydro-4aH-benzo[f]indazole-4a-carboxylate (112b)

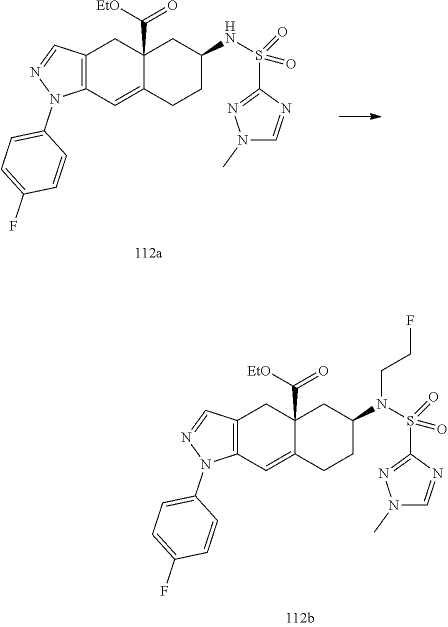

To a flask charged with ethyl (4aS,6S)-1-(4-fluorophenyl)-6-[(1-methyl-1,2,4-triazol-3-yl)sulfonylamino]-5,6,7,8-tetrahydro-4H-benzo[f]indazole-4a-carboxylate (112a) (156 mg, 0.31 mmol) were added DMF (6 mL) and cesium carbonate (203.09 mg, 0.62 mmol). The reaction mixture was stirred at rt for 10 min and 1-fluoro-2-iodoethane (0.13 mL, 1.25 mmol) was added. After the reaction was stirred at rt for 3 h, additional fluoride (50 uL) and Cs₂CO₃ (50 mg) were added. The reaction was stirred overnight, then quenched (water) and extracted (EtOAc). The organic phase was washed (brine), dried (Na₂SO₄) and concentrated. The residue was purified by silica gel column chromatography (SiO₂, 0%-100% EtOAc/hexanes, gradient elution) to provide the title compound (112b) (127 mg, 74.6% yield). m/z (ESI, +ve ion)=547.2 [M+H]⁺.

### Step C: N-(2-Fluoroethyl)-N-((4aS,6S)-1-(4-fluorophenyl)-4a-(5-(trifluoromethyl)thiazole-2-carbonyl)-4,4a,5,6,7,8-hexahydro-1H-benzo[f]indazol-6-yl)-1-methyl-1H-1,2,4-triazole-3-sulfonamide (112)

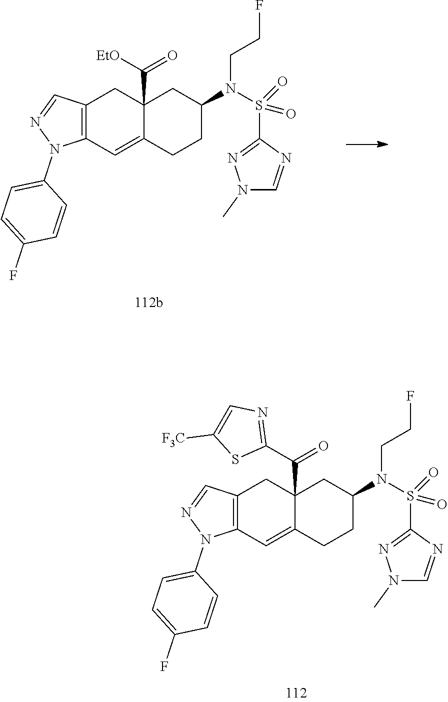

Ethyl (4aS,6S)-6-[2-fluoroethyl-[(1-methyl-1,2,4-triazol-3-yl)sulfonyl]amino]-1-(4-fluorophenyl)-5,6,7,8-tetrahydro-4H-benzo[f]indazole-4a-carboxylate (112b) (50 mg, 0.09 mmol) was azeotroped with toluene in a 25 mL flask and the flask was put under high vacuum. To a separate dried flask under an argon balloon was added anhydrous ether (1.3 mL) and n-butyllithium (0.31 mL, 0.50 mmol), followed dropwise addition of 2-bromo-5-(trifluoromethyl)-1,3-thiazole (0.07 mL, 0.55 mmol) in ether (0.5 mL) at −78° C. The solution was stirred at −78° C. for 30 min.

The flask with 112b (50 mg, 0.09 mmol) was flushed with argon and a mixed solvent THF (0.3 mL) and anhydrous ether (0.7 mL) was added. This solution was added dropwise to the lithiated species in the first flask at −78° C. After the reaction was stirred for 1 h, it was quenched with water/sat NH₄Cl, extracted (EtOAc), washed (brine), dried (MgSO₄), and concentrated. The residue was purified by silica gel chromatography (SiO₂, 0%-100% EtOAc/hexanes, gradient elution) to afford the title compound (112) (36 mg, 60% yield) as a yellow solid. ¹H NMR (400 MHz, Chloroform-d) δ ppm 8.24-8.29 (m, 1H), 8.17 (s, 1H), 7.41-7.48 (m, 2H), 7.30-7.34 (m, 1H), 7.13-7.21 (m, 2H), 6.50 (d, J=1.75 Hz, 1H), 4.68-4.76 (m, 1H), 4.55-4.65 (m, 1H), 4.35-4.46 (m, 1H), 4.06 (s, 3H), 3.92 (d, J=16.66 Hz, 1H), 3.61-3.66 (m, 1H), 3.54-3.60 (m, 1H), 3.24 (d, J=16.81 Hz, 1H), 2.40-2.63 (m, 4H), 1.83-1.94 (m, 2H); m/z (ESI, +ve ion)=654.1 [M+H]⁺.

### Example 113. ((4aS,6S)-1-(4-Fluorophenyl)-6-((1-methyl-1H-pyrazol-3-yl)sulfonyl)-1,4,5,6,7,8-hexahydro-4aH-benzo[f]indazol-4a-yl)(4-(trifluoromethyl)pyridin-2-yl)methanone (113)

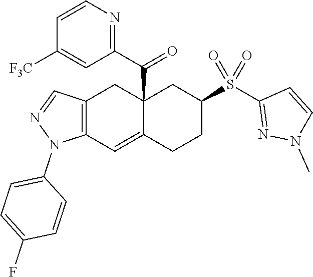 [SMILES: Cn1ccc(S(=O)(=O)[C@H]2CCC3=Cc4c(cnn4-c4ccc(F)cc4)C[C@]3(C(=O)c3cc(C(F)(F)F)ccn3)C2)n1]

### Step A: Ethyl(4aS,6R)-1-(4-fluorophenyl)-6-hydroxy-1,4,5,6,7,8-hexahydro-4aH-benzo[f]indazole-4a-carboxylate (113a)

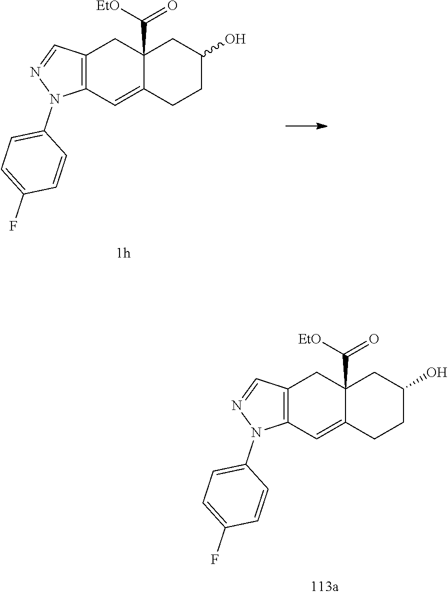 [SMILES: CCOC(=O)[C@]12Cc3cnn(-c4ccc(F)cc4)c3C=C1CCC(O)C2 ; CCOC(=O)[C@]12Cc3cnn(-c4ccc(F)cc4)c3C=C1CC[C@@H](O)C2]

Separation of a diastereomeric mixture of ethyl (4aS,6R)-1-(4-fluorophenyl)-6-hydroxy-1,4,5,6,7,8-hexahydro-4aH-benzo[f]indazole-4a-carboxylate and ethyl (4aS,6S)-1-(4-fluorophenyl)-6-hydroxy-1,4,5,6,7,8-hexahydro-4aH-benzo[f]indazole-4a-carboxylate (h) (6.0 g) by silica gel column chromatography (330 g SiO₂, 10-40% acetone/hexanes, gradient elution) afford the title compound (113a) (second eluting isomer, 3.3 g) as an orange foamy solid. m/z (ESI, +ve ion)=356.7 [M+H]⁺.

### Step B: 3-((4-Methoxybenzyl)thio)-1-methyl-1H-pyrazole (113b)

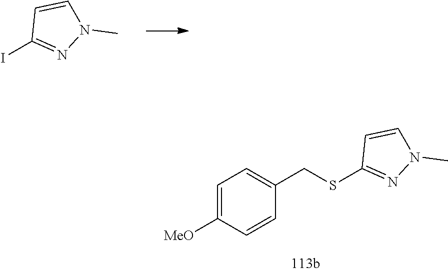

Around bottom flask was charged with 3-iodo-1-methyl-1H-pyrazole (5.08 g, 24.4 mmol), 4-methoxybenzylmercaptan (4.40 mL, 31.8 mmol), xantphos (707 mg, 1.20 mmol) and 1,4-dioxane (130 mL), N-ethyldiisopropylamine (8.50 mL, 48.8 mmol) and tris(dibenzylideneacetone)dipalladium(0) (559 mg, 0.60 mmol) were successively added under argon. The flask was purged with argon and the mixture was heated at 90° C. for 5 h. After the reaction mixture was cooled down to rt, it was filtered over a pad of Celite and rinsed with EtOAc. The filtrate was concentrated under reduced pressure and the orange residue was purified by silica gel column chromatography (120 g SiO₂, 20% to 50% EtOAc/hexanes, gradient elution) to provide the title compound (113b) (5.30 g, 93%) as an orange solid. m/z (ESI, +ve ion)=235.1 [M+H]⁺.

### Step C: 1,2-Bis(1-methyl-1H-pyrazol-3-yl)disulfane (113c)

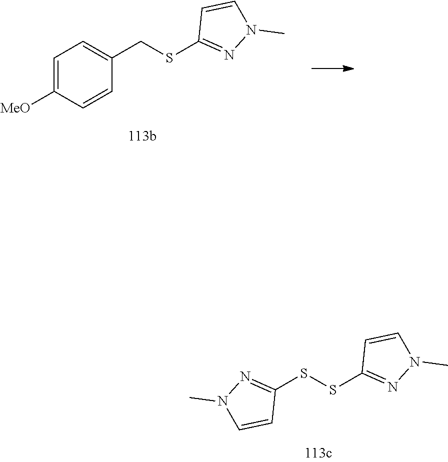

A pressure tube was charged with a solution of 3-((4-methoxybenzyl)thio)-1-methyl-H-pyrazole (113b) (5.30 g, 22.6 mmol) in TFA (70 mL) and the solution was heated at 100° C. for 20 h. After the solution was cooled down to rt, TFA was removed under reduced pressure and the residue was azeotroped with toluene. This deep greenish residue was dissolved in DCM (200 mL) and iodobenzene diacetate (7.29 g, 22.6 mmol) was added in one portion. After the mixture was stirred at rt for 10 min, the reaction was quenched (sat. aq. NaHCO₃, 10% aq. NaS₂O₃). The resulting solution was stirred at rt for 20 min and extracted (DCM). The combined organic layers were washed (brine), dried (Na₂SO₄), and concentrated under reduced pressure. The residue was purified by silica gel column chromatography (220 g SiO₂, 2% to 5% MeOH/DCM, gradient elution, followed by another column chromatography (80 g SiO₂, 50% to 100% EtOAc/hexanes, gradient elution) to provide the title compound (113c) (1.80 g, 70%) as an orange solid. m/z (ESI, +ve ion)=227.1 [M+H]⁺.

### Step D: Ethyl (4aS,6S)-1-(4-fluorophenyl)-6-((1-methyl-1H-pyrazol-3-yl)thio)-1,4,5,6,7,8-hexahydro-4aH-benzo[f]indazole-4a-carboxylate (113d)

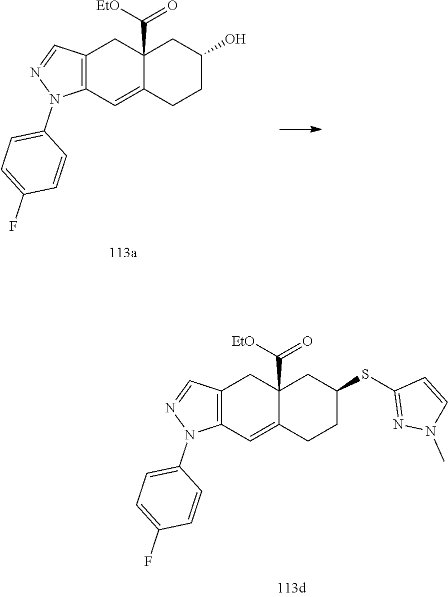 [SMILES: CCOC(=O)[C@]12Cc3cnn(-c4ccc(F)cc4)c3C=C1CC[C@@H](O)C2 ; CCOC(=O)[C@]12Cc3cnn(-c4ccc(F)cc4)c3C=C1CC[C@H](Sc1ccn(C)n1)C2]

To a mixture of ethyl (4aS,6R)-1-(4-fluorophenyl)-6-hydroxy-1,4,5,6,7,8-hexahydro-4aH-benzo[f]indazole-4a-carboxylate (113a) (2.59 g, 7.3 mmol) and 1,2-bis(1-methyl-H-pyrazol-3-yl)disulfane (113c) (3.78 g, 16.7 mmol) in toluene (36 mL) was added a solution of n-Bu₃P (4.20 mL, 16.7 mmol) in toluene (18 mL) dropwise over the period of 15 min under argon. The mixture was heated at 100° C. for 20 h. After the mixture was cooled down to rt, toluene was removed under reduced pressure. Purification of the residue by silica gel column chromatography (330 g SiO₂, 10% to 30% acetone/hexanes, gradient elution) provided the title compound (113d) (2.79 g, 85%) as a yellow foamy solid. m/z (ESI, +ve ion)=453.1 [M+H]⁺.

### Step E: ((4aS,6S)-1-(4-Fluorophenyl)-6-((1-methyl-1H-pyrazol-3-yl)thio)-1,4,5,6,7,8-hexahydro-4aH-benzo[f]indazol-4a-yl)methanol (113e)

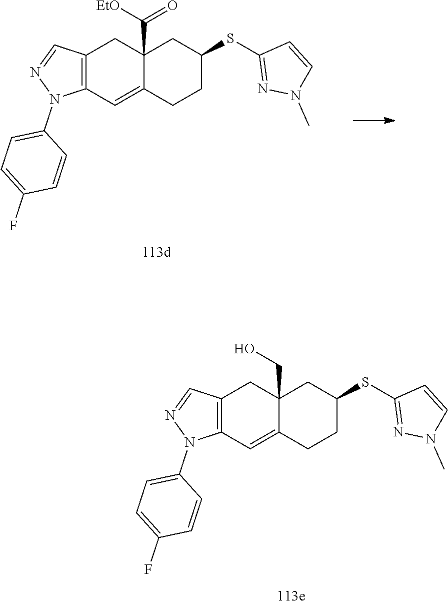 [SMILES: CCOC(=O)[C@]12Cc3cnn(-c4ccc(F)cc4)c3C=C1CC[C@H](Sc1ccn(C)n1)C2 ; Cn1ccc(S[C@H]2CCC3=Cc4c(cnn4-c4ccc(F)cc4)C[C@]3(CO)C2)n1]

To a stirred solution of ethyl (4aS,6S)-1-(4-fluorophenyl)-6-((1-methyl-1H-pyrazol-3-yl)thio)-1,4,5,6,7,8-hexahydro-4aH-benzo[f]indazole-4a-carboxylate (113d) (2.92 g, 6.45 mmol) in diethyl ether (100 mL) was added lithium aluminum hydride (1.0 M in THF, 8.4 mL, 8.4 mmol) at 0° C. Gas evolution was observed and the reaction mixture became white cloudy suspension. The mixture was stirred at 0° C. for 10 min and then EtOAc (70 mL) was added. After the mixture was allowed to warm to rt and stirred for another 20 min, it was quenched (water) and the resulting suspension was filtered through a small pad of Celite. The organic phase was washed (brine), dried (Na₂SO₄), and concentrated under reduced pressure to afford the title compound (113e) (2.65 g, 100%) as an off-white foamy solid. m/z (ESI, +ve ion)=411.1 [M+H]⁺.

### Step F: ((4aS,6S)-1-(4-Fluorophenyl)-6-((1-methyl-1H-pyrazol-3-yl)sulfonyl)-1,4,5,6,7,8-hexahydro-4aH-benzo[f]indazol-4a-yl)(4-(trifluoromethyl)pyridin-2-yl)methanone (113)

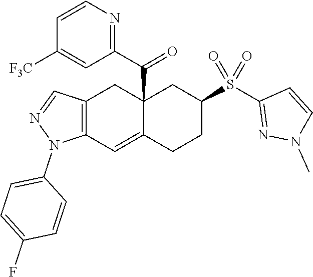 [SMILES: Cn1ccc(S(=O)(=O)[C@H]2CCC3=Cc4c(cnn4-c4ccc(F)cc4)C[C@]3(C(=O)c3cc(C(F)(F)F)ccn3)C2)n1]

The title compound was prepared from ((4aS,6S)-1-(4-fluorophenyl)-6-((1-methyl-1H-pyrazol-3-yl)thio)-1,4,5,6,7,8-hexahydro-4aH-benzo[f]indazol-4a-yl)methanol (113e) by procedures similar to those described in Example 3, Steps D, E, F and G. ¹H NMR (400 MHz, Chloroform-d) δ 8.87 (1H, d, J=5.2 Hz), 8.05 (1H, m), 7.69-7.67 (1H, m), 7.48 (1H, d, J=2.0 Hz), 7.43-7.39 (2H, m), 7.27 (1H, s), 7.17-7.13 (2H, m), 6.76 (1H, d, J=2.4 Hz), 6.48 (1H, d, J=1.6 Hz), 4.05 (1H, d, J=16.4 Hz), 4.02 (3H, s), 3.70-3.62 (1H, m), 3.09 (1H, dd, J=14.0, 2.4 Hz), 3.02 (1H, d, J=16.4 Hz), 2.67-2.45 (3H, m), 2.29-2.20 (1H, m), 1.92-1.82 (1H, m); m/z (ESI, +ve ion)=586.2 [M+H]⁺.

### Example 114. ((4aS,6S)-6-Fluoro-1-(4-fluorophenyl)-6-((1-methyl-1H-pyrazol-3-yl)sulfonyl)-1,4,5,6,7,8-hexahydro-4aH-benzo[f]indazol-4a-yl)(4-(trifluoromethyl)pyridin-2-yl)methanone (114)

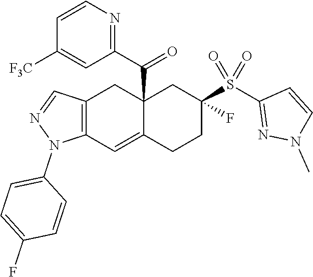 [SMILES: Cn1ccc(S(=O)(=O)[C@@]2(F)CCC3=Cc4c(cnn4-c4ccc(F)cc4)C[C@]3(C(=O)c3cc(C(F)(F)F)ccn3)C2)n1]

### Step A: ((4aS,6S)-1-(4-Fluorophenyl)-6-((1-methyl-1H-pyrazol-3-yl)sulfonyl)-1,4,5,6,7,8-hexahydro-4aH-benzo[f]indazol-4a-yl)methanol (114a)

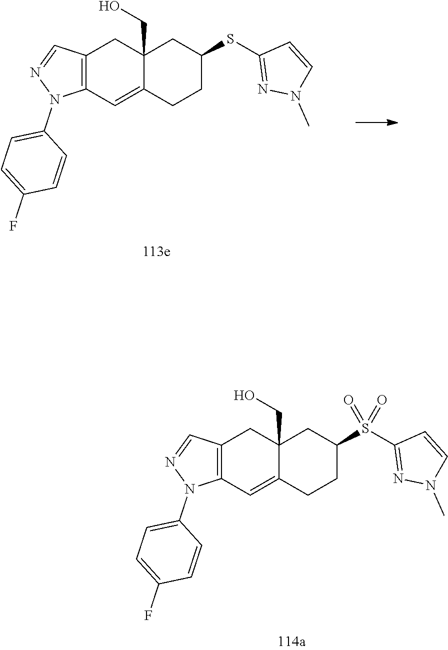 [SMILES: Cn1ccc(S[C@H]2CCC3=Cc4c(cnn4-c4ccc(F)cc4)C[C@]3(CO)C2)n1 ; Cn1ccc(S(=O)(=O)[C@H]2CCC3=Cc4c(cnn4-c4ccc(F)cc4)C[C@]3(CO)C2)n1]

To a stirred solution of ((4aS,6S)-1-(4-Fluorophenyl)-6-((1-methyl-H-pyrazol-3-yl)thio)-1,4,5,6,7,8-hexahydro-4aH-benzo[f]indazol-4a-yl)methanol (113e) (1.00 g, 2.44 mmol) in MeOH (6.7 mL), water (6.7 mL), and THF (13.3 mL) was added oxone (1.85 g, 12.2 mmol) in one portion at rt. The mixture was heated at 40° C. for 5 h. After the mixture was cooled down to 0° C., it was quenched (10% aq. Na₂S₂O₃, sat. aq. NaHCO₃) and extracted (EtOAc). The organic layers were washed (brine), dried (Na₂SO₄), and concentrated under reduced pressure. Purification of the residue by silica gel column chromatography (40 g SiO₂, 0% to 15% MeOH/EtOAc, gradient elution) provided the title compound (114a) (1.09 g, 100%) as an off-white foamy solid. m/z (ESI, +ve ion)=443.2 [M+H]⁺.

### Step B: (4aS,6S)-4a-(((tert-Butyldimethylsilyl)oxy)methyl)-1-(4-fluorophenyl)-6-((1-methyl-1H-pyrazol-3-yl)sulfonyl)-4,4a,5,6,7,8-hexahydro-1H-benzo[f]indazole (114b)

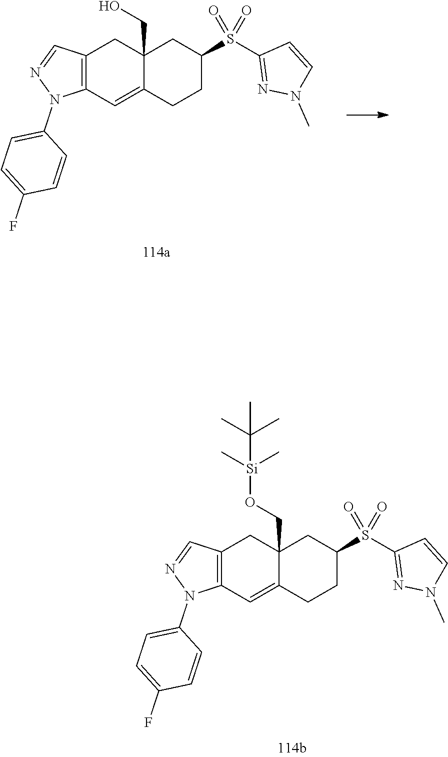 [SMILES: Cn1ccc(S(=O)(=O)[C@H]2CCC3=Cc4c(cnn4-c4ccc(F)cc4)C[C@]3(CO)C2)n1 ; Cn1ccc(S(=O)(=O)[C@H]2CCC3=Cc4c(cnn4-c4ccc(F)cc4)C[C@]3(CO[Si](C)(C)C(C)(C)C)C2)n1]

To a solution of ((4aS,6S)-1-(4-fluorophenyl)-6-((1-methyl-H-pyrazol-3-yl)sulfonyl)-1,4,5,6,7,8-hexahydro-4aH-benzo[f]indazol-4a-yl)methanol (114a) (1.17 g, 2.66 mmol) in DMF (12 mL) at 0° C. were added tert-butyldimethylsilyl chloride (1.39 g, 9.25 mmol) and imidazole (846 mg, 12.4 mmol) successively. After the solution was allowed to warm to rt and stirred for 2 h, it was quenched (water) and extracted (EtOAc). The organic layers were washed (brine), dried (Na₂SO₄), and concentrated under reduced pressure. Purification of the residue by silica gel column chromatography (24 g SiO₂, 20% to 100% EtOAc/hexane, gradient elution) provided the title compound (114b) (1.19 g, 81%) as a white foamy solid. m/z (ESI, +ve ion)=557.3 [M+H]⁺.

### Step C: (4aS,6S)-4a-(((tert-Butyldimethylsilyl)oxy)methyl)-6-fluoro-1-(4-fluorophenyl)-6-((1-methyl-1H-pyrazol-3-yl)sulfonyl)-4,4a,5,6,7,8-hexahydro-1H-benzo[f]indazole (114c)

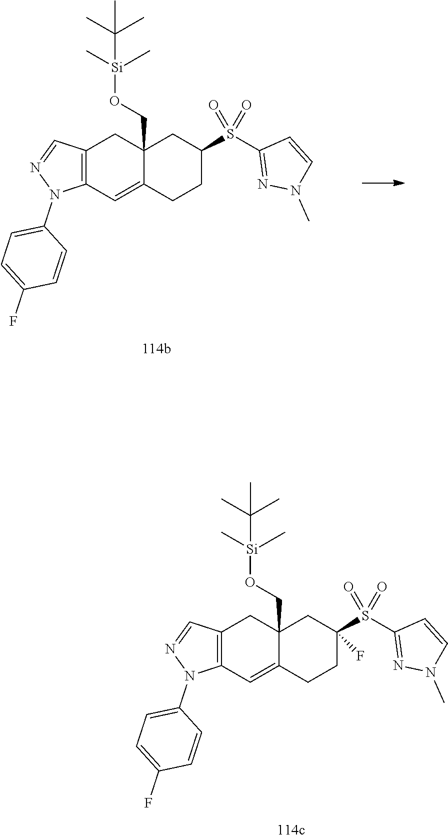 [SMILES: Cn1ccc(S(=O)(=O)[C@H]2CCC3=Cc4c(cnn4-c4ccc(F)cc4)C[C@]3(CO[Si](C)(C)C(C)(C)C)C2)n1 ; Cn1ccc(S(=O)(=O)[C@@]2(F)CCC3=Cc4c(cnn4-c4ccc(F)cc4)C[C@]3(CO[Si](C)(C)C(C)(C)C)C2)n1]

To a stirred solution of (4aS,6S)-4a-(((tert-butyldimethylsilyl)oxy)methyl)-1-(4-fluorophenyl)-6-((1-methyl-1H-pyrazol-3-yl)sulfonyl)-4,4a,5,6,7,8-hexahydro-1H-benzo[f]indazole (114b) (600 mg, 1.08 mmol) in THF (7.0 mL) at −78° C. was added n-butyllithium solution (1.6 M in hexane, 0.94 mL, 1.51 mmol) dropwise. The reaction was stirred at −78° C. for 30 min and then a solution of N-fluorobenzenesulfonimide (476 mg, 1.51 mmol) in THF (7.0 mL) was added dropwise. The resulting solution was stirred at −78° C. for 10 min and was quenched (water). The dry ice bath was removed and sat. aq. NH₄Cl solution was added. After the solution was allowed to warm to rt, it was extracted (3×EtOAc). The combined organic layers were washed (brine), dried (Na₂SO₄), and concentrated under reduced pressure. Purification of the residue by silica gel column chromatography (120 g SiO₂, 15% to 60% EtOAc/hexane, a gradient elution) provided the title compound (114c) (332 mg, 54%) as a white foamy solid. m/z (ESI, +ve ion) 575.2 [M+H]⁺.

### Step D: ((4aS,6S)-6-Fluoro-1-(4-fluorophenyl)-6-((1-methyl-1H-pyrazol-3-yl)sulfonyl)-1,4,5,6,7,8-hexahydro-4aH-benzo[f]indazol-4a-yl)methanol (114d)

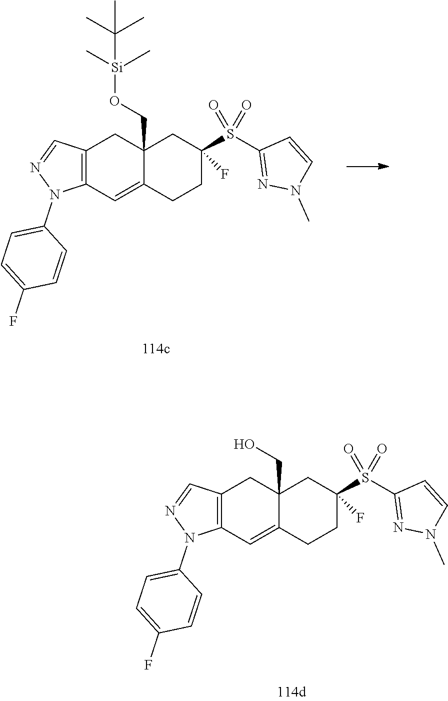 [SMILES: Cn1ccc(S(=O)(=O)[C@@]2(F)CCC3=Cc4c(cnn4-c4ccc(F)cc4)C[C@]3(CO[Si](C)(C)C(C)(C)C)C2)n1 ; Cn1ccc(S(=O)(=O)[C@@]2(F)CCC3=Cc4c(cnn4-c4ccc(F)cc4)C[C@]3(CO)C2)n1]

To a stirred solution of (4aS,6S)-4a-(((tert-butyldimethylsilyl)oxy)methyl)-6-fluoro-1-(4-fluorophenyl)-6-((1-methyl-1H-pyrazol-3-yl)sulfonyl)-4,4a,5,6,7,8-hexahydro-1H-benzo[f]indazole (114c) (320 mg, 0.557 mmol) in MeOH (40 mL) was added 3N aq. HCl solution (5.6 mL, 16.7 mmol) dropwise. The reaction was stirred at rt for 4 h, quenched (sat. aq. NaHCO₃) and extracted (EtOAc). The organic layers were washed (brine), dried (Na₂SO₄), and concentrated under reduced pressure. Purification of the residue by silica gel column chromatography (24 g SiO₂, 50% to 100% EtOAc/hexane, gradient elution) provided the title compound (114d) (256 mg, 100%) as a white foamy solid. m/z (ESI, +ve ion)=461.1 [M+H]⁺.

### Step E: ((4aS,6S)-6-Fluoro-1-(4-fluorophenyl)-6-((1-methyl-1H-pyrazol-3-yl)sulfonyl)-1,4,5,6,7,8-hexahydro-4aH-benzo[f]indazol-4a-yl)(4-(trifluoromethyl)pyridin-2-yl)methanone (114)

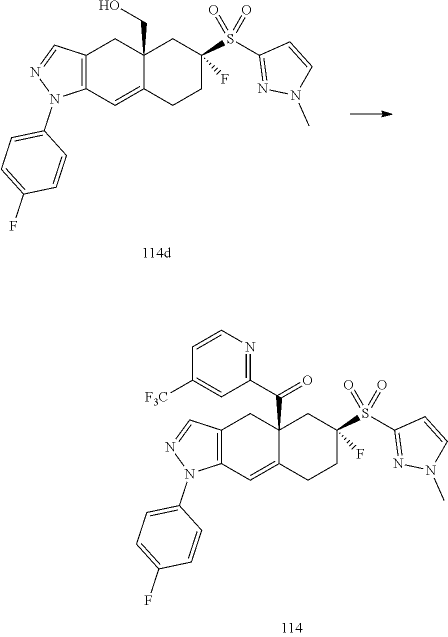 [SMILES: Cn1ccc(S(=O)(=O)[C@@]2(F)CCC3=Cc4c(cnn4-c4ccc(F)cc4)C[C@]3(CO)C2)n1 ; Cn1ccc(S(=O)(=O)[C@@]2(F)CCC3=Cc4c(cnn4-c4ccc(F)cc4)C[C@]3(C(=O)c3cc(C(F)(F)F)ccn3)C2)n1]

The title compound was prepared from ((4aS,6S)-6-fluoro-1-(4-fluorophenyl)-6-((1-methyl-1H-pyrazol-3-yl)sulfonyl)-1,4,5,6,7,8-hexahydro-4aH-benzo[f]indazol-4a-yl)methanol (114d) by procedures similar to those described in Example 3, Steps D, E, and F. H NMR (400 MHz, Chloroform-d) δ 8.88 (1H, d, J=5.2 Hz), 8.07 (1H, m), 7.70-7.68 (1H, m), 7.54 (1H, d, J=2.4 Hz), 7.43-7.39 (2H, m), 7.25 (1H, s), 7.17-7.13 (2H, m), 6.83 (1H, d, J=2.4 Hz), 6.53 (1H, s), 4.08 (1H, d, J=17.4 Hz), 4.06 (3H, s), 3.36-3.11 (3H, m), 2.90-2.67 (2H, m), 2.63-2.57 (1H, m), 2.01-1.87 (1H, m); m/z (ESI, +ve ion)=604.2 [M+H]⁺.

### Example 115. ((4aS,6S)-1-(4-Fluorophenyl)-6-((2-(2,2,2-trifluoroethyl)-2H-1,2,3-triazol-4-yl)sulfonyl)-1,4,5,6,7,8-hexahydro-4aH-benzo[f]indazol-4a-yl)(4-(trifluoromethyl)pyridin-2-yl)methanone (115)

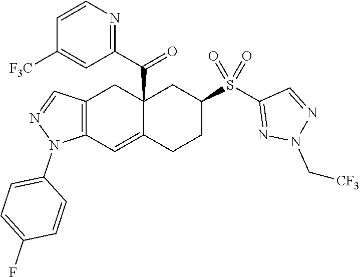 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)n1)C2]

### STEP A: Ethyl(4aS,6S)-6-((1H-1,2,3-triazol-5-yl)thio)-1-(4-fluorophenyl)-1,4,5,6,7,8-hexahydro-4aH-benzo[f]indazole-4a-carboxylate (115a)

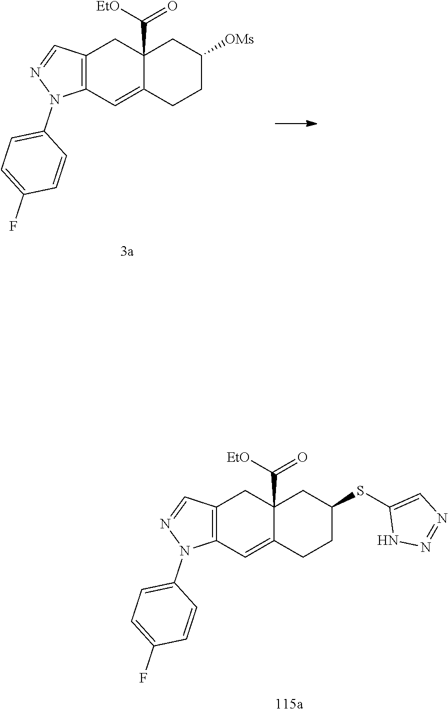 [SMILES: CCOC(=O)[C@]12Cc3cnn(-c4ccc(F)cc4)c3C=C1CC[C@@H](OS(C)(=O)=O)C2 ; CCOC(=O)[C@]12Cc3cnn(-c4ccc(F)cc4)c3C=C1CC[C@H](Sc1cnn[nH]1)C2]

To a stirred solution of ethyl (4aS,6R)-1-(4-fluorophenyl)-6-((methylsulfonyl)oxy)-1,4,5,6,7,8-hexahydro-4aH-benzo[f]indazole-4a-carboxylate (3a) (1.19 g, 2.74 mmol) in DMF (40 mL) was added sodium 1H-1,2,3-triazole-4-thiolate (2.0 g, 16.4 mmol) at rt under argon. The resulting mixture was heated at 90° C. for 3.5 h. After the reaction was cooled down to rt, it was poured into sat. aq. NH₄Cl solution. The solution was extracted (EtOAc) and the organic layers were washed (brine), dried (Na₂SO₄), and concentrated under reduced pressure. Purification of the residue by silica gel column chromatography (80 g SiO₂, 40% to 70% EtOAc/hexanes, gradient elution) provided ethyl (4aS,6S)-6-((1H-1,2,3-triazol-5-yl)thio)-1-(4-fluorophenyl)-1,4,5,6,7,8-hexahydro-4aH-benzo[f]indazole-4a-carboxylate (115a) (765 mg, 64%) as an off-white solid. m/z (ESI, +ve ion)=440.2 [M+H]⁺.

### Step B: Ethyl (4aS,6S)-1-(4-fluorophenyl)-6-((2-(2,2,2-trifluoroethyl)-2H-1,2,3-triazol-4-yl)thio)-1,4,5,6,7,8-hexahydro-4aH-benzo[f]indazole-4a-carboxylate (115b)

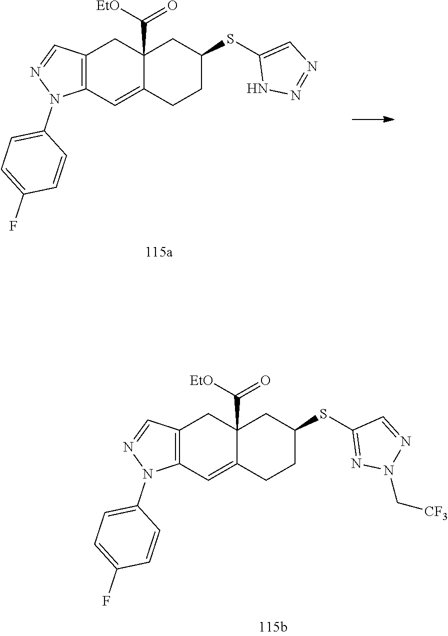 [SMILES: CCOC(=O)[C@]12Cc3cnn(-c4ccc(F)cc4)c3C=C1CC[C@H](Sc1cnn[nH]1)C2 ; CCOC(=O)[C@]12Cc3cnn(-c4ccc(F)cc4)c3C=C1CC[C@H](Sc1cnn(CC(F)(F)F)n1)C2]

To a stirred solution of ethyl (4aS,6S)-6-((1H-1,2,3-triazol-5-yl)thio)-1-(4-fluorophenyl)-1,4,5,6,7,8-hexahydro-4aH-benzo[f]indazole-4a-carboxylate (115a) (310 mg, 0.71 mmol) in DMF (7 mL) was added cesium carbonate (689 mg, 2.12 mmol). The mixture was stirred at rt for 3 min and 2,2,2-trifluoroethyl trifluoromethanesulfonate (0.20 mmol, 1.42 mmol) was added. After the resulting mixture was stirred at rt for 1 h, it was poured into water, extracted (EtOAc). The organic layers were washed (water and brine), dried (Na₂SO₄), and concentrated under reduced pressure. Purification of the residue by silica gel column chromatography (40 g SiO₂, 15% to 60% acetone/hexanes, gradient elution) afforded the title compound (115b) (148 mg, 40%) as an off-white solid. m/z (ESI, +ve ion)=522.2 [M+H]⁺.

### Step C: (4aS,6S)-1-(4-Fluorophenyl)-6-((2-(2,2,2-trifluoroethyl)-2H-1,2,3-triazol-4-yl)thio)-1,4,5,6,7,8-hexahydro-4aH-benzo[f]indazole-4a-carbaldehyde (115c)

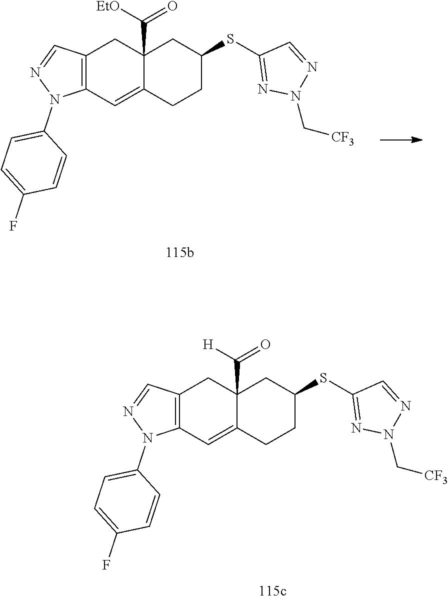 [SMILES: CCOC(=O)[C@]12Cc3cnn(-c4ccc(F)cc4)c3C=C1CC[C@H](Sc1cnn(CC(F)(F)F)n1)C2 ; O=C[C@]12Cc3cnn(-c4ccc(F)cc4)c3C=C1CC[C@H](Sc1cnn(CC(F)(F)F)n1)C2]

The title compound was prepared from ethyl (4aS,6S)-1-(4-fluorophenyl)-6-((2-(2,2,2-trifluoroethyl)-2H-1,2,3-triazol-4-yl)thio)-1,4,5,6,7,8-hexahydro-4aH-benzo[f]indazole-4a-carboxylate (115b) by procedures similar to those described in Example 3, Steps C and D. m/z (ESI, +ve ion)=478.1 [M+H]⁺.

### Step D: ((4aS,6S)-1-(4-Fluorophenyl)-6-((2-(2,2,2-trifluoroethyl)-2H-1,2,3-triazol-4-yl)thio)-1,4,5,6,7,8-hexahydro-4aH-benzo[f]indazol-4a-yl)(4-(trifluoromethyl)pyridin-2-yl)methanol (115d)

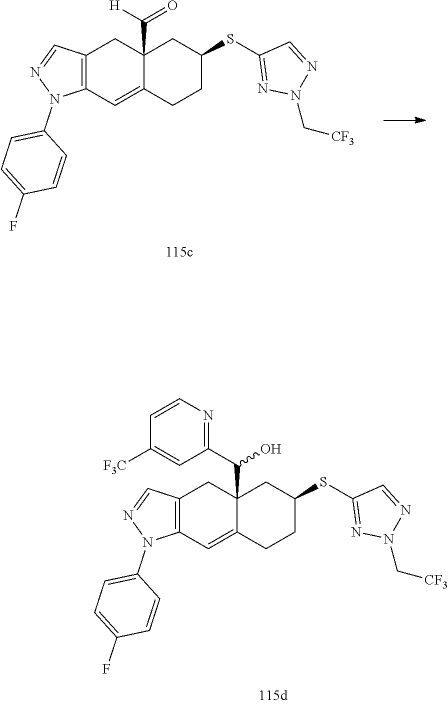 [SMILES: O=C[C@]12Cc3cnn(-c4ccc(F)cc4)c3C=C1CC[C@H](Sc1cnn(CC(F)(F)F)n1)C2 ; OC(c1cc(C(F)(F)F)ccn1)[C@]12Cc3cnn(-c4ccc(F)cc4)c3C=C1CC[C@H](Sc1cnn(CC(F)(F)F)n1)C2]

To a flask with diethyl ether (1.5 mL) was added isopropylmagnesium chloride solution (2.0 M in THF, 0.44 mL, 0.88 mmol) at 0° C., followed by the dropwise addition of 2-bromo-4-(trifluoromethyl)pyridine (0.11 mL, 0.88 mmol). After the mixture was stirred at 0° C. for 50 min, a solution of (4aS,6S)-1-(4-fluorophenyl)-6-((2-(2,2,2-trifluoroethyl)-2H-1,2,3-triazol-4-yl)thio)-1,4,5,6,7,8-hexahydro-4aH-benzo[f]indazole-4a-carbaldehyde (115c) (105 mg, 0.22 mmol) in THF (1.0 mL) dropwise was added. The resulting mixture was stirred at 0° C. for 15 min and then allowed to warm to rt. After the reaction at rt for 2 h, it was quenched (sat. aq. NH₄Cl) and extracted (EtOAc). The organic layer was washed (brine), dried (Na₂SO₄), and concentrated under reduced pressure. Purification of the residue by silica gel column chromatography (24 g SiO₂, 0.5% to 3% MeOH/DCM, gradient elution) provided ((4aS,6S)-1-(4-fluorophenyl)-6-((2-(2,2,2-trifluoroethyl)-2H-1,2,3-triazol-4-yl)thio)-1,4,5,6,7,8-hexahydro-4aH-benzo[f]indazol-4a-yl)(4-(trifluoromethyl)pyridin-2-yl)methanol (115d) (57.9 mg, 42%) as a yellow solid. m/z (ESI, +ve ion) 625.2 [M+H]⁺.

### Step E: ((4aS,6S)-1-(4-Fluorophenyl)-6-((2-(2,2,2-trifluoroethyl)-2H-1,2,3-triazol-4-yl)sulfonyl)-1,4,5,6,7,8-hexahydro-4aH-benzo[f]indazol-4a-yl)(4-(trifluoromethyl)pyridin-2-yl)methanone (115)

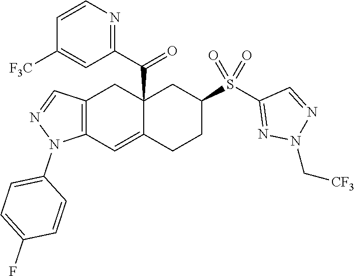 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)n1)C2]

The title compound was prepared from ((4aS,6S)-1-(4-fluorophenyl)-6-((2-(2,2,2-trifluoroethyl)-2H-1,2,3-triazol-4-yl)thio)-1,4,5,6,7,8-hexahydro-4aH-benzo[f]indazol-4a-yl)(4-(trifluoromethyl)pyridin-2-yl)methanol (115d) by procedures similar to those described in Example 3, Steps F and G. ¹H NMR (400 MHz, Chloroform-d) δ 8.76 (1H, d, J=4.8 Hz), 8.06 (1H. s), 7.99 (1H, m), 7.62 (1H, ddd, J=4.8, 1.6, 0.6 Hz), 7.36-7.33 (2H, m), 7.21 (1H, s), 7.11-7.06 (2H, m), 7.44 (1H, d, J=1.2 Hz), 5.06 (2H, q, J=7.7 Hz), 3.95 (1H, d, J=16.4 Hz), 3.69-3.61 (1H, m), 2.98 (1H, dd, J=14.0, 2.8 Hz), 2.91 (1H, d, J=16.8 Hz), 2.56-2.43 (3H, m), 2.23-2.14 (1H, m), 1.90-1.80 (1H, m); m/z (ESI, +ve ion)=655.2 [M+H]⁺.

### Example 116. ((4aS,6R)-1-(4-Fluorophenyl)-6-methyl-6-(phenylsulfonyl)-1,4,5,6,7,8-hexahydro-4aH-benzo[f]indazol-4a-yl)(pyridin-2-yl)methanone or ((4aS,6S)-1-(4-fluorophenyl)-6-methyl-6-(phenylsulfonyl)-1,4,5,6,7,8-hexahydro-4aH-benzo[f]indazol-4a-yl)(pyridin-2-yl)methanone (116)

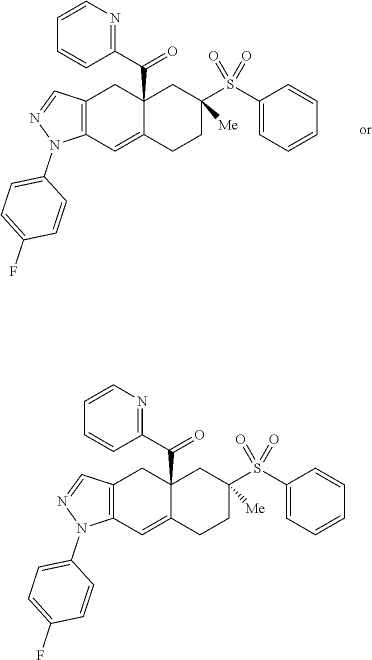 [SMILES: C[C@@]1(S(=O)(=O)c2ccccc2)CCC2=Cc3c(cnn3-c3ccc(F)cc3)C[C@]2(C(=O)c2ccccn2)C1 ; C[C@]1(S(=O)(=O)c2ccccc2)CCC2=Cc3c(cnn3-c3ccc(F)cc3)C[C@]2(C(=O)c2ccccn2)C1]

### Step A: Ethyl (4aS,6R)-1-(4-fluorophenyl)-6-(phenylthio)-1,4,5,6,7,8-hexahydro-4aH-benzo[f]indazole-4a-carboxylate (116a)

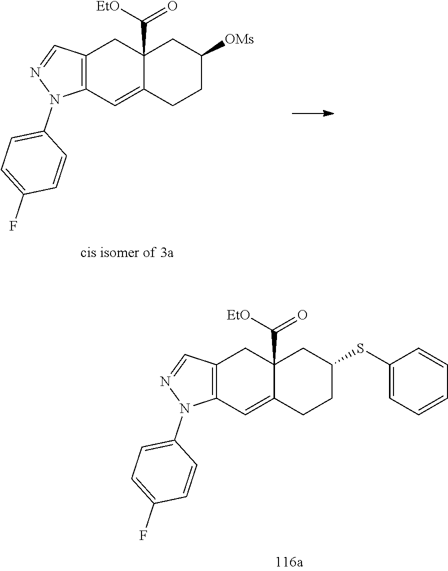 [SMILES: CCOC(=O)[C@]12Cc3cnn(-c4ccc(F)cc4)c3C=C1CC[C@H](OS(C)(=O)=O)C2 ; CCOC(=O)[C@]12Cc3cnn(-c4ccc(F)cc4)c3C=C1CC[C@@H](Sc1ccccc1)C2]

To a stirred suspension of sodium hydride (60% in mineral oil, 574 mg, 14.4 mmol) in DMF (14 mL) was added thiophenol (1.6 mL, 15.8 mmol) at rt under Ar. The mixture was stirred at rt until gas evolution was ceased. To the thiolate solution prepared above was added a solution of ethyl (4aS,6S)-1-(4-fluorophenyl)-6-((methylsulfonyl)oxy)-1,4,5,6,7,8-hexahydro-4aH-benzo[f]indazole-4a-carboxylate (cis isomer of 3a) (1.56 g, 3.6 mmol) in DMF (7 mL). After the mixture was heated at 55° C. for 1 h and cooled down to rt, it was poured into sat. aq. NH₄Cl solution and extracted (EtOAc). The organic layer was washed (water, brine), dried (Na₂SO₄, and concentrated under reduced pressure. Purification of the residue by silica gel column chromatography (120 g SiO₂, 5% to 30% EtOAc/hexanes, gradient elution) provided ethyl (4aS,6R)-1-(4-fluorophenyl)-6-(phenylthio)-1,4,5,6,7,8-hexahydro-4aH-benzo[f]indazole-4a-carboxylate (116a) (790 mg, 49%) as a white solid. m/z (ESI, +ve ion)=449.2 [M+H]⁺.

### Step B: ((4aS,6R)-1-(4-Fluorophenyl)-6-(phenylthio)-1,4,5,6,7,8-hexahydro-4aH-benzo[f]indazol-4a-yl)methanol (116b)

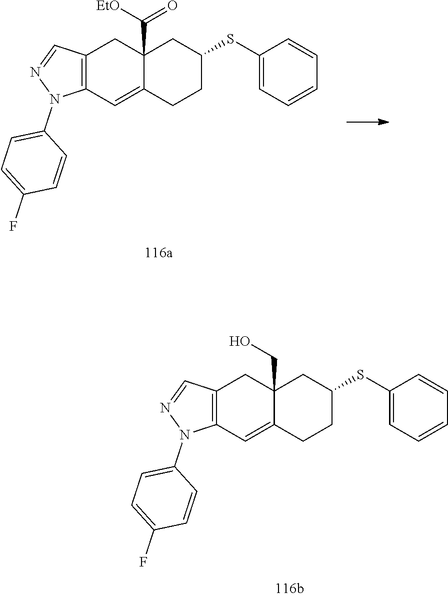 [SMILES: CCOC(=O)[C@]12Cc3cnn(-c4ccc(F)cc4)c3C=C1CC[C@@H](Sc1ccccc1)C2 ; OC[C@]12Cc3cnn(-c4ccc(F)cc4)c3C=C1CC[C@@H](Sc1ccccc1)C2]

To a stirred solution of ethyl (4aS,6R)-1-(4-fluorophenyl)-6-(phenylthio)-1,4,5,6,7,8-hexahydro-4aH-benzo[f]indazole-4a-carboxylate (116a) (790 mg, 1.76 mmol) in diethyl ether (24 mL) was added lithium aluminum hydride (1.0 M in THF, 1.95 mL, 1.95 mmol) at 0° C. The mixture was stirred at 0° C. for 20 min and EtOAc (20 mL) was added. The mixture was allowed to warm to rt and stirred for 20 min. The reaction was quenched (water) and the resulting suspension was filtered through a small pad of Celite and the organic phase was washed (water, brine), dried (Na₂SO₄), and concentrated under reduced pressure. Purification of the residue by silica gel column chromatography (40 g SiO₂, 30% to 100% EtOAc/hexanes, gradient elution) provided ethyl ((4aS,6R)-1-(4-fluorophenyl)-6-(phenylthio)-1,4,5,6,7,8-hexahydro-4aH-benzo[f]indazol-4a-yl)methanol (116b) (678 mg, 95%) as a white foamy solid. m/z (ESI, +ve ion)=407.1 [M+H]⁺.

### Step C: ((4aS,6R)-1-(4-Fluorophenyl)-6-(phenylsulfonyl)-1,4,5,6,7,8-hexahydro-4aH-benzo[f]indazol-4a-yl)methanol (116c)

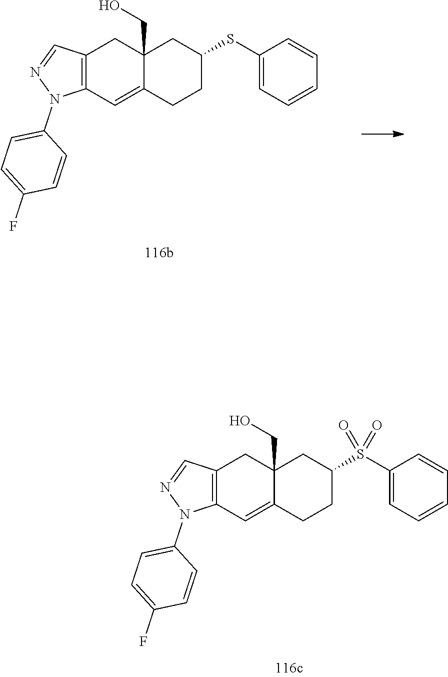 [SMILES: OC[C@]12Cc3cnn(-c4ccc(F)cc4)c3C=C1CC[C@@H](Sc1ccccc1)C2 ; O=S(=O)(c1ccccc1)[C@@H]1CCC2=Cc3c(cnn3-c3ccc(F)cc3)C[C@]2(CO)C1]

To a stirred solution of ((4aS,6R)-1-(4-fluorophenyl)-6-(phenylthio)-1,4,5,6,7,8-hexahydro-4aH-benzo[f]indazol-4a-yl)methanol (116b) (345 mg, 0.85 mmol) in MeOH (2.2 mL), water (2.2 mL), and THF (4.4 mL) was added oxone (646 mg, 4.2 mmol) in one portion at rt. The mixture was heated at 40° C. for 1 h. After the reaction was cooled down to rt, it was quenched (10% aq. Na₂S₂O₃, sat. aq. NaHCO₃) and extracted (EtOAc). The organic layer was washed (brine), dried (Na₂SO₄), and concentrated under reduced pressure. Purification of the residue by silica gel column chromatography (24 g SiO₂, 50% to 100% EtOAc/hexanes, gradient elution) provided ((4aS,6R)-1-(4-fluorophenyl)-6-(phenylsulfonyl)-1,4,5,6,7,8-hexahydro-4aH-benzo[f]indazol-4a-yl)methanol (116c) (376 mg, 100%) as a white solid. m/z (ESI, +ve ion)=439.1 [M+H]⁺.

### Step D: (4aS,6R)-4a-(((tert-Butyldimethylsilyl)oxy)methyl)-1-(4-fluorophenyl)-6-(phenylsulfonyl)-4,4a,5,6,7,8-hexahydro-1H-benzo[f]indazole (116d)

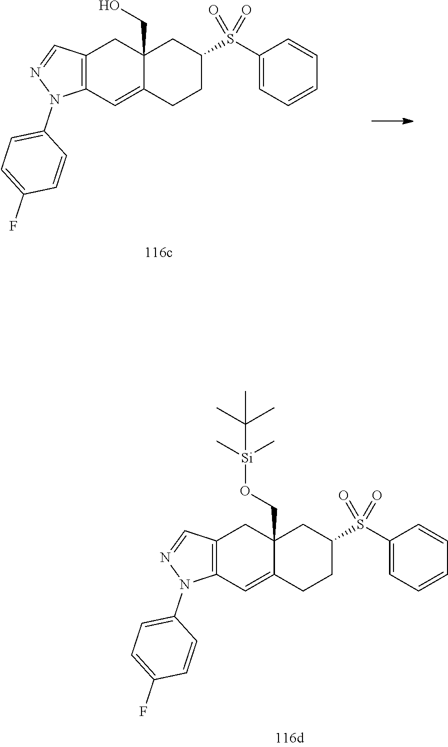 [SMILES: O=S(=O)(c1ccccc1)[C@@H]1CCC2=Cc3c(cnn3-c3ccc(F)cc3)C[C@]2(CO)C1 ; CC(C)(C)[Si](C)(C)OC[C@]12Cc3cnn(-c4ccc(F)cc4)c3C=C1CC[C@@H](S(=O)(=O)c1ccccc1)C2]

To a solution of ((4aS,6R)-1-(4-fluorophenyl)-6-(phenylsulfonyl)-1,4,5,6,7,8-hexahydro-4aH-benzo[f]indazol-4a-yl)methanol (116c) (392 mg, 0.89 mmol) in DMF (4 mL) at 0° C. were added tert-butyldimethylsilyl chloride (270 mg, 1.79 mmol) and imidazole (164 mg, 2.41 mmol) successively. After the solution was allowed to warm to rt and stirred for 1 h, it was quenched (water) and extracted (EtOAc). The organic layer was washed (brine), dried (Na₂SO₄), and concentrated under reduced pressure. Purification of the residue by silica gel column chromatography (40 g SiO₂, 10% to 50% EtOAc/hexane, a gradient elution) provided (4aS,6R)-4a-(((tert-butyldimethylsilyl)oxy)methyl)-1-(4-fluorophenyl)-6-(phenylsulfonyl)-4,4a,5,6,7,8-hexahydro-1H-benzo[f]indazole (116d) (416 mg, 84%) as a white foamy solid. m/z (ESI, +ve ion)=553.3 [M+H]⁺.

### Step E: (4aS,6R)-4a-(((tert-Butyldimethylsilyl)oxy)methyl)-1-(4-fluorophenyl)-6-methyl-6-(phenylsulfonyl)-4,4a,5,6,7,8-hexahydro-1H-benzo[f]indazole or (4aS,6S)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole (116e-1) and (4aS,6R)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole or (4aS,6S)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole (116e-2)

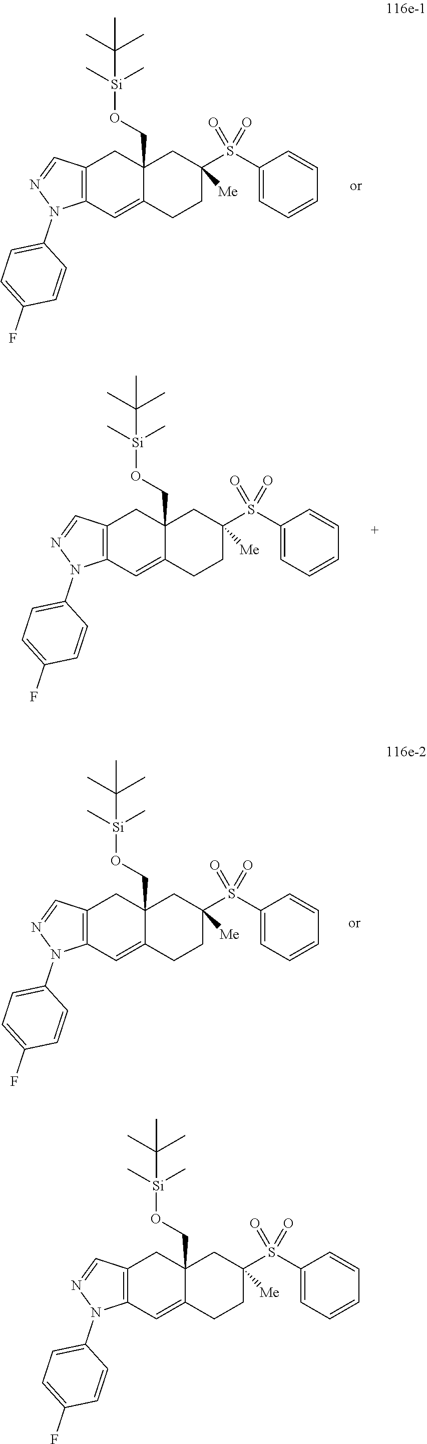 [SMILES: CC(C)(C)[Si](C)(C)OC[C@]12Cc3cnn(-c4ccc(F)cc4)c3C=C1CC[C@@](C)(S(=O)(=O)c1ccccc1)C2 ; CC(C)(C)[Si](C)(C)OC[C@]12Cc3cnn(-c4ccc(F)cc4)c3C=C1CC[C@](C)(S(=O)(=O)c1ccccc1)C2 ; CC(C)(C)[Si](C)(C)OC[C@]12Cc3cnn(-c4ccc(F)cc4)c3C=C1CC[C@@](C)(S(=O)(=O)c1ccccc1)C2 ; CC(C)(C)[Si](C)(C)OC[C@]12Cc3cnn(-c4ccc(F)cc4)c3C=C1CC[C@](C)(S(=O)(=O)c1ccccc1)C2]

To a stirred solution of (4aS,6R)-4a-(((tert-butyldimethylsilyl)oxy)methyl)-1-(4-fluorophenyl)-6-(phenylsulfonyl)-4,4a,5,6,7,8-hexahydro-1H-benzo[f]indazole (116d) (276 mg, 0.50 mmol) in THF (4.0 mL) at −78° C. was added n-butyllithium solution (1.6 M in hexane, 0.47 mL, 0.75 mmol) dropwise. After the reaction was stirred at −78° C. for 30 min, a solution of iodomethane (106 mg, 0.75 mmol) in THF (2.0 mL) was added dropwise. The resulting solution was stirred at −78° C. for 30 min and then quenched with water. The dry ice bath was removed and sat. aq. NH₄Cl solution was added. After the solution was allowed to warm to rt, it was extracted (EtOAc). The organic layer was washed (brine), dried (Na₂SO₄), and concentrated under reduced pressure. Purification of the residue by silica gel column chromatography (40 g SiO₂, 20% to 40% MTBE/hexane, gradient elution) provided the title compound (116e-1) (first eluting isomer, 190 mg, 67%) and compound (116e-2) (second eluting isomer, 104 mg, 37%). m/z (ESI, +ve ion) 567.2 [M+H]⁺.

### Step F: ((4aS,6R)-1-(4-Fluorophenyl)-6-methyl-6-(phenylsulfonyl)-1,4,5,6,7,8-hexahydro-4aH-benzo[f]indazol-4a-yl)(pyridin-2-yl)methanone or ((4aS,6S)-1-(4-fluorophenyl)-6-methyl-6-(phenylsulfonyl)-1,4,5,6,7,8-hexahydro-4aH-benzo[f]indazol-4a-yl)(pyridin-2-yl)methanone (116)

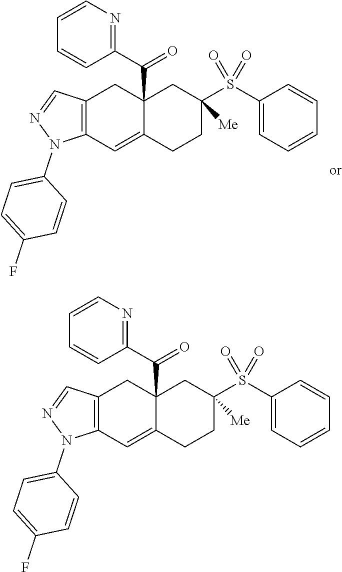 [SMILES: C[C@@]1(S(=O)(=O)c2ccccc2)CCC2=Cc3c(cnn3-c3ccc(F)cc3)C[C@]2(C(=O)c2ccccn2)C1 ; C[C@]1(S(=O)(=O)c2ccccc2)CCC2=Cc3c(cnn3-c3ccc(F)cc3)C[C@]2(C(=O)c2ccccn2)C1]

The title compound was prepared from (4aS,6R)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole or (4aS,6S)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole (116e-1) by procedures similar to those described in Example 114, Steps D and E. ¹H NMR (400 MHz, Chloroform-d) δ 8.49-8.47 (1H, m), 7.89-7.87 (2H, m), 7.75-7.63 (3H, m), 7.59-7.55 (2H, m), 7.39-7.35 (3H, m), 7.22 (1H, s), 7.17-7.13 (2H, m), 6.46 (1H, d, J=2.4 Hz), 4.09-4.01 (2H, m), 2.91 (1H, d, J=16.4 Hz), 2.71-2.59 (2H, m), 2.46 (1H, d, J=13.6 Hz), 2.15 (1H, td, J=12.8, 4.8 Hz), 1.85-1.80 (1H, m), 1.10 (3H, s); m/z (ESI, +ve ion)=528.2 [M+H]⁺.

### Example 117: ((4aS,6R)-1-(4-Fluorophenyl)-6-methyl-6-(phenylsulfonyl)-1,4,5,6,7,8-hexahydro-4aH-benzo[f]indazol-4a-yl)(pyridin-2-yl)methanone or ((4aS,6S)-1-(4-fluorophenyl)-6-methyl-6-(phenylsulfonyl)-1,4,5,6,7,8-hexahydro-4aH-benzo[f]indazol-4a-yl)(pyridin-2-yl)methanone (117)

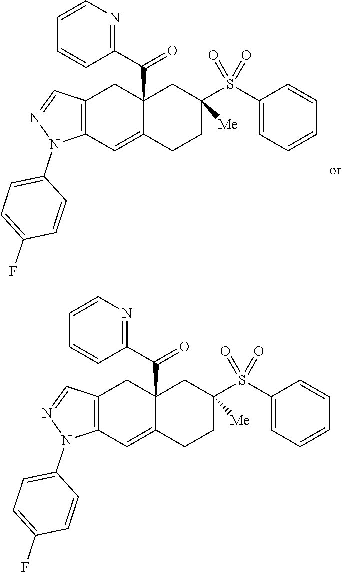 [SMILES: C[C@@]1(S(=O)(=O)c2ccccc2)CCC2=Cc3c(cnn3-c3ccc(F)cc3)C[C@]2(C(=O)c2ccccn2)C1 ; C[C@]1(S(=O)(=O)c2ccccc2)CCC2=Cc3c(cnn3-c3ccc(F)cc3)C[C@]2(C(=O)c2ccccn2)C1]

The title compound was prepared from (4aS,6R)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole or (4aS,6S)-4a-(((tert-butyldimethylsilyl)oxy)methyl)-1-(4-fluorophenyl)-6-methyl-6-(phenylsulfonyl)-4,4a,5,6,7,8-hexahydro-1H-benzo[f]indazole (116e-2) by procedure similar to that described in Example 116, Step F. ¹H NMR (400 MHz, Chloroform-d) 8.67-8.66 (1H, m), 7.90 (1H, dt, J=8.0, 1.0 Hz), 7.81 (1H, td, J=7.8, 1.9 Hz), 7.76-7.74 (2H, m), 7.65 (1H, tt, J=7.6, 1.4 Hz), 7.55-7.50 (2H, m), 7.47-7.42 (3H, m), 7.24 (1H, s), 7.18-7.13 (2H, m), 6.52 (1H, m), 4.27 (1H, d, J=16.4 Hz), 3.66 (1H, d, J=15.2 Hz), 2.94 (1H, d, J=16.4 Hz), 2.83-2.77 (1H, m), 2.54-2.48 (2H, m), 2.34 (1H, d, J=15.2 Hz), 1.62-1.56 (1H, m), 1.41 (3H, s); m/z (ESI, +ve ion)=528.1 [M+H]⁺.

### Example 118. ((4aR,6S)-1-(4-Fluorophenyl)-6-(((1-methyl-1H-pyrazol-4-yl)sulfonyl)methyl)-1,4,5,6,7,8-hexahydro-4aH-benzo[f]indazol-4a-yl)(4-(trifluoromethyl)pyridin-2-yl)methanone (118)

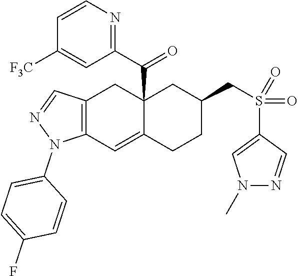 [SMILES: Cn1cc(S(=O)(=O)C[C@H]2CCC3=Cc4c(cnn4-c4ccc(F)cc4)C[C@]3(C(=O)c3cc(C(F)(F)F)ccn3)C2)cn1]

### Step A: (S)-(1-(4-Fluorophenyl)-1,4,7,8-tetrahydrospiro[benzo[f]indazole-6,2′-[1,3]dioxolan]-4a(5H)-yl)methanol (118a)

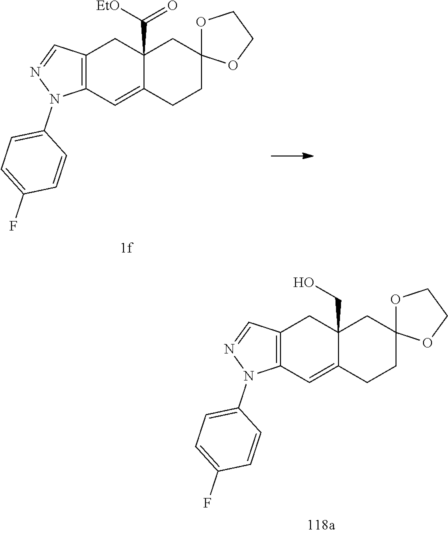 [SMILES: CCOC(=O)[C@]12Cc3cnn(-c4ccc(F)cc4)c3C=C1CCC1(C2)OCCO1 ; OC[C@]12Cc3cnn(-c4ccc(F)cc4)c3C=C1CCC1(C2)OCCO1]

To a stirred solution of ethyl (S)-1-(4-fluorophenyl)-1,4,7,8-tetrahydrospiro[benzo[f]indazole-6,2′-[1,3]dioxolane]-4a(5H)-carboxylate (f) (14.4 g, 36.1 mmol) in diethyl ether (300 mL) was added lithium aluminum hydride (1.0 M in THF, 47.0 mL, 47.0 mmol) at 0° C. The mixture was stirred at 0° C. for 20 min and EtOAc (200 mL) was added. After the mixture was allowed to warm to rt and stirred for 20 min, it was quenched (water) and the resulting suspension was filtered through a small pad of Celite. The organic phase was washed (water, brine), dried (Na₂SO₄, and concentrated under reduced pressure to afford (S)-(1-(4-fluorophenyl)-1,4,7,8-tetrahydrospiro[benzo[f]indazole-6,2′-[1,3]dioxolan]-4a(5H)-yl)methanol (118a) (12.9 g, 100%) as a yellow foamy solid. m/z (ESI, +ve ion)=357.2 [M+H]⁺.

### Step B: (S)-1-(4-Fluorophenyl)-4a-(hydroxymethyl)-1,4,4a,5,7,8-hexahydro-6H-benzo[f]indazol-6-one (118b)

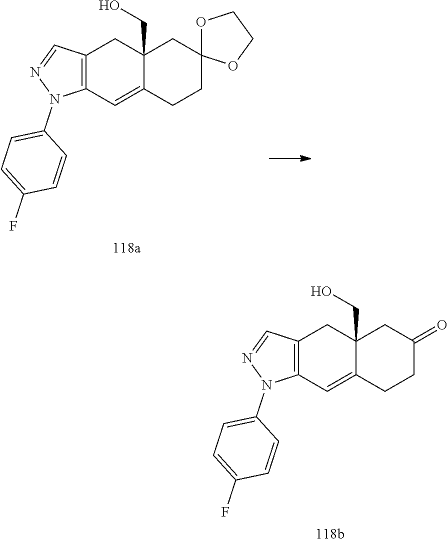 [SMILES: OC[C@]12Cc3cnn(-c4ccc(F)cc4)c3C=C1CCC1(C2)OCCO1 ; O=C1CCC2=Cc3c(cnn3-c3ccc(F)cc3)C[C@]2(CO)C1]

To a stirred solution of (S)-(1-(4-fluorophenyl)-1,4,7,8-tetrahydrospiro[benzo[f]indazole-6,2′-[1,3]dioxolan]-4a(5H)-yl)methanol (118a) (12.9 g, 36.1 mmol) in acetone (200 mL) was added 4 N aqueous HCl (108 mL, 433 mmol). The reaction mixture was stirred at rt overnight and neutralized by 2N NaOH (55 mL) and sat. aq. NaHCO₃ (500 mL). Acetone was removed under reduced pressure and the remaining was extracted (EtOAc). The organic layer was washed (brine), dried (Na₂SO₄), and concentrated under reduced pressure. Purification of the residue by silica gel chromatography (220 g SiO₂, 50% to 100% EtOAc/hexane, gradient elution) provided (S)-1-(4-fluorophenyl)-4a-(hydroxymethyl)-1,4,4a,5,7,8-hexahydro-6H-benzo[f]indazol-6-one (118b) (11.1 g, 98%) as a yellow solid. m/z (ESI, +ve ion) 313.1 [M+H]⁺.

### Step C: (S)-4a-(((tert-Butyldimethylsilyl)oxy)methyl)-1-(4-fluorophenyl)-1,4,4a,5,7,8-hexahydro-6H-benzo[f]indazol-6-one (118c)

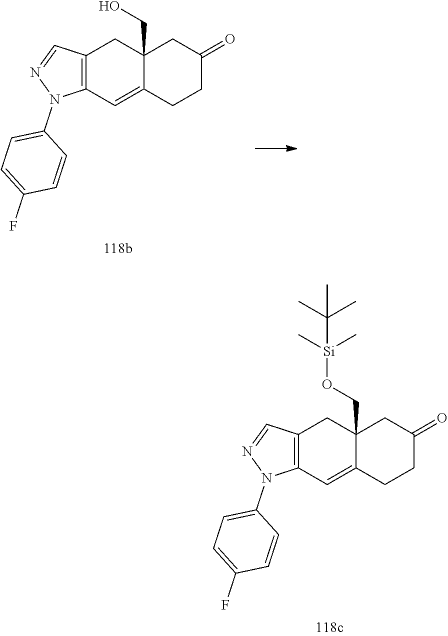 [SMILES: O=C1CCC2=Cc3c(cnn3-c3ccc(F)cc3)C[C@]2(CO)C1 ; CC(C)(C)[Si](C)(C)OC[C@@]12CC(=O)CCC1=Cc1c(cnn1-c1ccc(F)cc1)C2]

To a solution of (S)-1-(4-fluorophenyl)-4a-(hydroxymethyl)-1,4,4a,5,7,8-hexahydro-6H-benzo[f]indazol-6-one (118b) (4.0 g, 12.8 mmol) in DMF (55 mL) were added tert-butyldimethylsilyl chloride (6.76 g, 44.8 mmol) and imidazole (4.10 g, 60.2 mmol) successively at 0° C. After the solution was allowed to warm to rt and stirred overnight, it was quenched (water) and extracted (EtOAc). The organic layer was washed (brine), dried (Na₂SO₄), and concentrated under reduced pressure. Purification of the residue by silica gel column chromatography (220 g SiO₂, 10% to 40% EtOAc/hexane, gradient elution) provided (S)-4a-(((tert-butyldimethylsilyl)oxy)methyl)-1-(4-fluorophenyl)-1,4,4a,5,7,8-hexahydro-6H-benzo[f]indazol-6-one (118c) (4.82 g, 88%) as an orange gum. m/z (ESI, +ve ion)=427.2 [M+H]⁺.

### Step D: (S)-4a-(((tert-Butyldimethylsilyl)oxy)methyl)-1-(4-fluorophenyl)-6-(methoxymethylene)-4,4a,5,6,7,8-hexahydro-1H-benzo[f]indazole (118d)

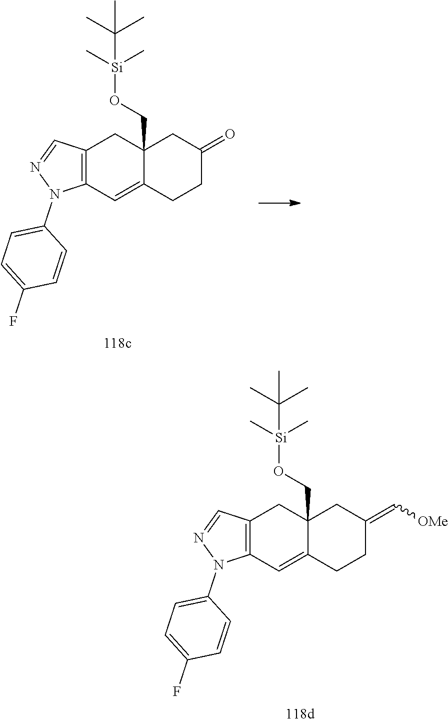 [SMILES: CC(C)(C)[Si](C)(C)OC[C@@]12CC(=O)CCC1=Cc1c(cnn1-c1ccc(F)cc1)C2 ; COC=C1CCC2=Cc3c(cnn3-c3ccc(F)cc3)C[C@]2(CO[Si](C)(C)C(C)(C)C)C1]

To a stirred solution of (S)-4a-(((tert-butyldimethylsilyl)oxy)methyl)-1-(4-fluorophenyl)-1,4,4a,5,7,8-hexahydro-6H-benzo[f]indazol-6-one (118c) (3.4 g, 8.0 mmol) and dimethyl diazomethylphosphonate (3.36 g, 22.4 mmol) in MeOH (14 mL) at 0° C. was added a solution of potassium tert-butoxide (2.5 g, 22.4 mml) in MeOH (12 mL) dropwise over the period of 10 min. After the mixture was allowed to warm to rt and stirred at rt for 30 min, it was poured into sat. aq. NaHCO₃ (80 mL). MeOH was removed under reduced pressure and the remaining was extracted (EtOAc). The organic layer was washed (brine), dried (Na₂SO₄), and concentrated under reduced pressure. Purification of the residue by silica gel column chromatography (220 g SiO₂, 0% to 15% EtOAc/hexane, gradient elution) provided (S)-4a-(((tert-butyldimethylsilyl)oxy)methyl)-1-(4-fluorophenyl)-6-(methoxymethylene)-4,4a,5,6,7,8-hexahydro-1H-benzo[f]indazole (118d) (3.08 g, 85%) as a colorless gum. m/z (ESI, +ve ion) 455.1 [M+H]⁺.

### Step E: (4aR)-4a-(((tert-Butyldimethylsilyl)oxy)methyl)-1-(4-fluorophenyl)-4,4a,5,6,7,8-hexahydro-1H-benzo[f]indazole-6-carbaldehyde (118e)

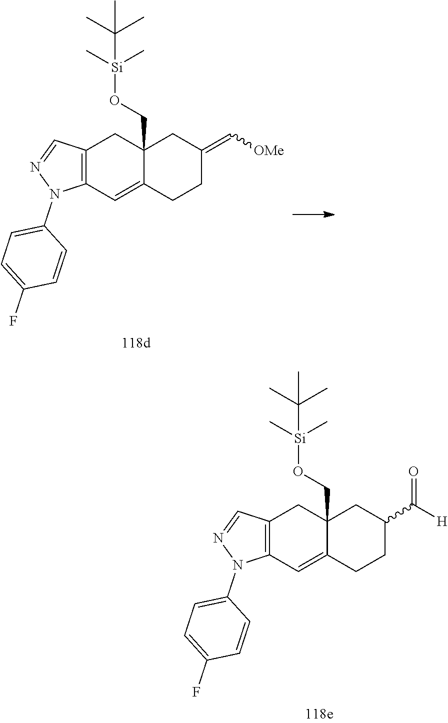 [SMILES: COC=C1CCC2=Cc3c(cnn3-c3ccc(F)cc3)C[C@]2(CO[Si](C)(C)C(C)(C)C)C1 ; CC(C)(C)[Si](C)(C)OC[C@]12Cc3cnn(-c4ccc(F)cc4)c3C=C1CCC(C=O)C2]

To a stirred solution of (S)-4a-(((tert-butyldimethylsilyl)oxy)methyl)-1-(4-fluorophenyl)-6-(methoxymethylene)-4,4a,5,6,7,8-hexahydro-1H-benzo[f]indazole (118d) (2.10 g, 4.62 mmol) in wet DCM (220 mL) was added trichloroacetic acid (7.17 g, 43.9 mmol), followed by addition of water (0.45 mL) at rt. After the reaction was stirred at rt for 4 h, it was quenched (sat. aq. NaHCO₃) and extracted (DCM). The organic layer was washed (brine), dried (Na₂SO₄), and concentrated under reduced pressure. Purification of the residue by silica gel column chromatography (80 g SiO₂, 5% to 20% EtOAc/hexane, gradient elution) provided (4aR)-4a-(((tert-butyldimethylsilyl)oxy)methyl)-1-(4-fluorophenyl)-4,4a,5,6,7,8-hexahydro-1H-benzo[f]indazole-6-carbaldehyde (118e) as a diastereomeric mixture (cis:trans=2.2:1.0, 1.64 g, 81%). m/z (ESI, +ve ion)=441.1 [M+H]⁺.

### Step F: ((4aR,6S)-4a-(((tert-Butyldimethylsilyl)oxy)methyl)-1-(4-fluorophenyl)-4,4a,5,6,7,8-hexahydro-1H-benzo[f]indazol-6-yl)methanol (118f-1) and ((4aR,6R)-4a-(((tert-Butyldimethylsilyl)oxy)methyl)-1-(4-fluorophenyl)-4,4a,5,6,7,8-hexahydro-1H-benzo[f]indazol-6-yl)methanol (118f-2)

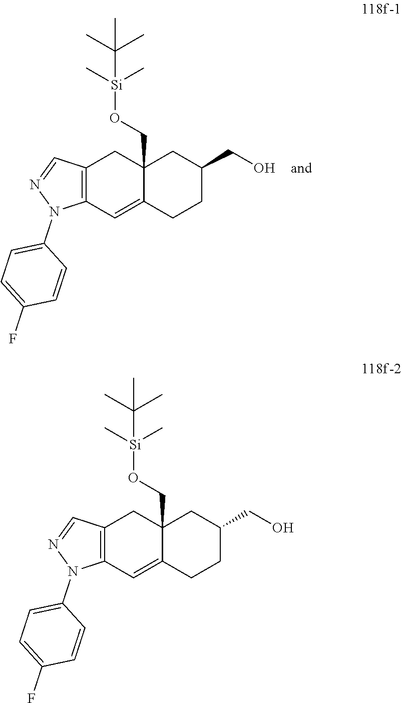 [SMILES: CC(C)(C)[Si](C)(C)OC[C@]12Cc3cnn(-c4ccc(F)cc4)c3C=C1CC[C@H](CO)C2 ; CC(C)(C)[Si](C)(C)OC[C@]12Cc3cnn(-c4ccc(F)cc4)c3C=C1CC[C@@H](CO)C2]

To a stirred solution of (4aR)-4a-(((tert-butyldimethylsilyl)oxy)methyl)-1-(4-fluorophenyl)-4,4a,5,6,7,8-hexahydro-1H-benzo[f]indazole-6-carbaldehyde (118e) (347 mg, 0.788 mmol) in MeOH (8 mL) was added NaBH₄ (44.7 mg, 1.18 mmol) at 0° C. The reaction mixture was allowed to warm to rt and stirred at rt for 15 min. After acetone (0.58 mL) was added, the resulting mixture was stirred at rt for another 30 min. The mixture was poured into water and the solution was extracted (EtOAc). The organic layer was washed (brine), dried (Na₂SO₄), and concentrated under reduced pressure. Purification of the residue by silica gel column chromatography (40 g SiO₂, 15% to 30% EtOAc/hexane, gradient elution) provided ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methanol (118f-1) (second eluting isomer, 227 mg, 65%) as a white foamy solid and ((4aR,6R)-4a-(((tert-butyldimethylsilyl)oxy)methyl)-1-(4-fluorophenyl)-4,4a,5,6,7,8-hexahydro-1H-benzo[f]indazol-6-yl)methanol (118f-2) (first eluting isomer, 116 mg, 33%) as a white foamy solid. m/z (ESI, +ve ion)=443.1 [M+H]⁺. The C₆ stereochemistry of 118f-1 and 118f-2 is randomly assigned.

### Step G: ((4aR,6S)-4a-(((tert-Butyldimethylsilyl)oxy)methyl)-1-(4-fluorophenyl)-4,4a,5,6,7,8-hexahydro-1H-benzo[f]indazol-6-yl)methyl methanesulfonate (118 g)

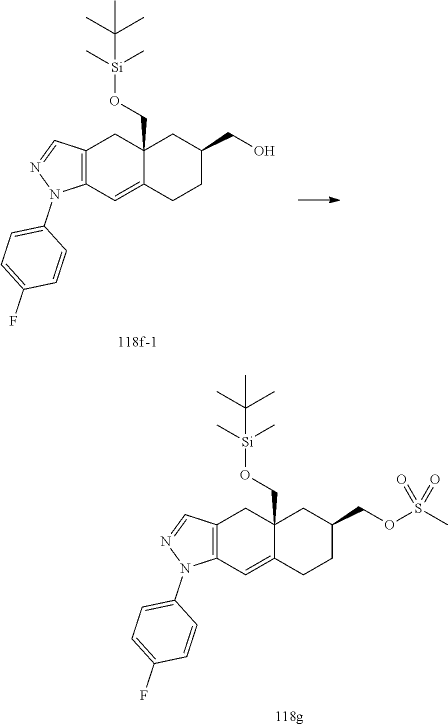 [SMILES: CC(C)(C)[Si](C)(C)OC[C@]12Cc3cnn(-c4ccc(F)cc4)c3C=C1CC[C@H](CO)C2 ; CC(C)(C)[Si](C)(C)OC[C@]12Cc3cnn(-c4ccc(F)cc4)c3C=C1CC[C@H](COS(C)(=O)=O)C2]

To a stirred solution of ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methanol (118f-1) (183 mg, 0.413 mmol) in DCM (4 mL) was added triethylamine (0.35 mL, 2.5 mmol). The mixture was cooled to 0° C. and methanesulfonyl chloride (80 L, 1.03 mmol) was added dropwise. After the reaction was allowed to warm to rt and stirred for 30 min, it was quenched (water) and extracted (DCM). The organics were washed (brine), dried (Na₂SO₄), and concentrated under reduced pressure. The residue was purified by silica gel column chromatography (40 g SiO₂, 20% to 40% EtOAc/hexane, a gradient elution) provided ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methyl methanesulfonate (118 g) (196 mg, 91%) as a colorless gum. m/z (ESI, +ve ion) 521.2 [M+H]⁺.

### Step H: (4aR,6S)-4a-(((tert-Butyldimethylsilyl)oxy)methyl)-1-(4-fluorophenyl)-6-(((1-methyl-1H-pyrazol-4-yl)thio)methyl)-4,4a,5,6,7,8-hexahydro-1H-benzo[f]indazole (118h)

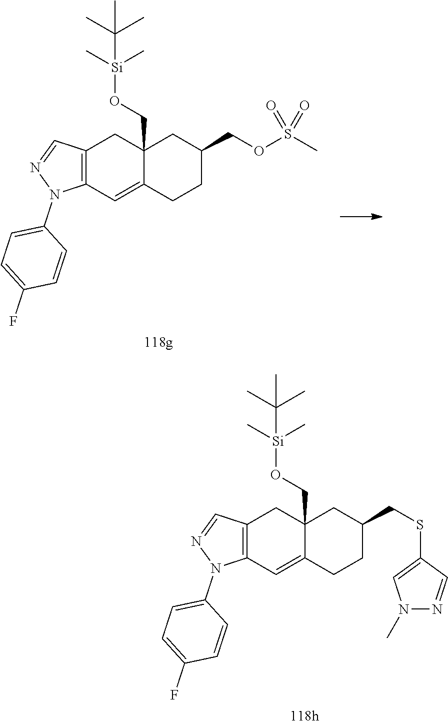 [SMILES: CC(C)(C)[Si](C)(C)OC[C@]12Cc3cnn(-c4ccc(F)cc4)c3C=C1CC[C@H](COS(C)(=O)=O)C2 ; Cn1cc(SC[C@H]2CCC3=Cc4c(cnn4-c4ccc(F)cc4)C[C@]3(CO[Si](C)(C)C(C)(C)C)C2)cn1]

To a stirred suspension of sodium hydride (60% in mineral oil, 22.6 mg, 0.57 mmol) in DMF (1 mL) was added 1-methylpyrazole-4-thiol (71 mg, 0.62 mmol) at rt under argon. The mixture was stirred at rt until gas evolution was ceased. To the thiolate solution prepared above was added a solution of ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methyl methanesulfonate (118 g) (98.0 mg, 0.188 mmol) in DMF (0.7 mL). After the mixture was heated at 50° C. for 30 min and cooled down to rt, it was poured into sat. aq. NH₄Cl solution and the solution was extracted (EtOAc). The organic layer was washed (water, brine), dried (Na₂SO₄), and concentrated under reduced pressure. Purification of the residue by silica gel column chromatography (24 g SiO₂, 30% to 50% EtOAc/hexanes, gradient elution) provided (4aR,6S)-4a-(((tert-butyldimethylsilyl)oxy)methyl)-1-(4-fluorophenyl)-6-(((1-methyl-1H-pyrazol-4-yl)thio)methyl)-4,4a,5,6,7,8-hexahydro-1H-benzo[f]indazole (118h) (89.5 mg, 88%) as a colorless gum. m/z (ESI, +ve ion)=539.2 [M+H]⁺.

### Step I: ((4aR,6S)-1-(4-Fluorophenyl)-6-(((1-methyl-1H-pyrazol-4-yl)thio)methyl)-1,4,5,6,7,8-hexahydro-4aH-benzo[f]indazol-4a-yl)methanol (118i)

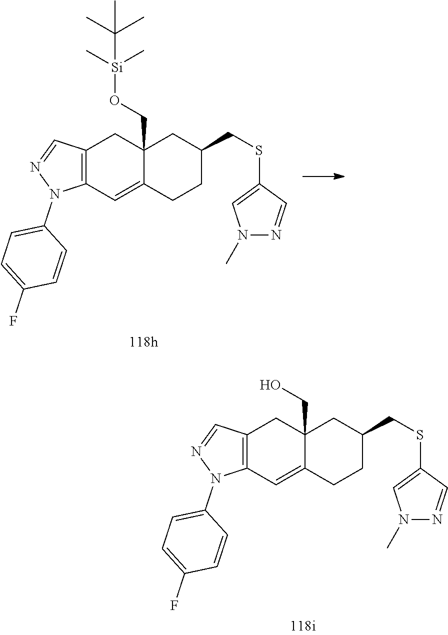 [SMILES: Cn1cc(SC[C@H]2CCC3=Cc4c(cnn4-c4ccc(F)cc4)C[C@]3(CO[Si](C)(C)C(C)(C)C)C2)cn1 ; Cn1cc(SC[C@H]2CCC3=Cc4c(cnn4-c4ccc(F)cc4)C[C@]3(CO)C2)cn1]

To a stirred solution of (4aR,6S)-4a-(((tert-butyldimethylsilyl)oxy)methyl)-1-(4-fluorophenyl)-6-(((1-methyl-1H-pyrazol-4-yl)thio)methyl)-4,4a,5,6,7,8-hexahydro-1H-benzo[f]indazole (118h) (89.5 mg, 0.166 mmol) in THF (5 mL) was added tetrabutylammonium fluoride solution (1.0 M in THF, 0.38 mL, 0.38 mmol) at rt. After the solution was stirred at rt for 3 h, THF was removed under reduced pressure and the residue was diluted (DCM). The organics were washed (water, brine), dried (Na₂SO₄), and concentrated under reduced pressure. Purification of the residue by silica gel column chromatography (12 g SiO₂, 70% to 100% EtOAc/hexanes, a gradient elution) provided ((4aR,6S)-1-(4-fluorophenyl)-6-(((1-methyl-1H-pyrazol-4-yl)thio)methyl)-1,4,5,6,7,8-hexahydro-4aH-benzo[f]indazo-4a-yl)methanol (118i) (40 mg, 57%) as a yellow gum. m/z (ESI, +ve ion)=425.2 [M+H]⁺.

### Step J: ((4aR,6S)-1-(4-Fluorophenyl)-6-(((1-methyl-1H-pyrazol-4-yl)sulfonyl)methyl)-1,4,5,6,7,8-hexahydro-4aH-benzo[f]indazol-4a-yl)(4-(trifluoromethyl)pyridin-2-yl)methanone (118)

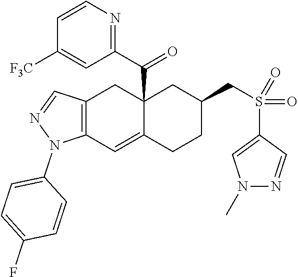 [SMILES: Cn1cc(S(=O)(=O)C[C@H]2CCC3=Cc4c(cnn4-c4ccc(F)cc4)C[C@]3(C(=O)c3cc(C(F)(F)F)ccn3)C2)cn1]

The title compound was prepared from ((4aR,6S)-1-(4-Fluorophenyl)-6-(((1-methyl-H-pyrazol-4-yl)thio)methyl)-1,4,5,6,7,8-hexahydro-4aH-benzo[f]indazol-4a-yl)methanol (118i) by procedures similar to those described in Example 3, Steps D, E, F and G. ¹H NMR (400 MHz, Chloroform-d) δ 8.85 (1H, d, J=4.4 Hz), 7.94 (1H, m), 7.82 (1H, m), 7.79 (1H, d, J=0.8 Hz), 7.65 (1H, ddd, J=4.8, 1.6, 0.6 Hz), 7.43-7.39 (2H, m), 7.25 (1H, s), 7.17-7.13 (2H, m), 7.46 (1H, d, J=2.0 Hz), 3.96 (3H, s), 3.95 (1H, d, J=16.4 Hz), 3.16 (1H, dd, J=14.0, 6.6 Hz), 3.09 (1H, dd, J=14.0, 6.2 Hz), 3.01 (1H, d, J=16.4 Hz), 2.66-2.52 (3H, m), 2.48-2.38 (2H, m), 1.96 (1H, m), 1.71-1.63 (1H, m); m/z (ESI, +ve ion)=600.1 [M+H]⁺. The C₆ stereochemistry of 118 is randomly assigned.

### Example 119. ((4aR,6R)-1-(4-Fluorophenyl)-6-(((1-methyl-1H-pyrazol-4-yl)sulfonyl)methyl)-1,4,5,6,7,8-hexahydro-4aH-benzo[f]indazol-4a-yl)(4-(trifluoromethyl)pyridin-2-yl)methanone (119)

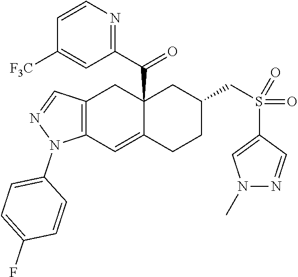 [SMILES: Cn1cc(S(=O)(=O)C[C@@H]2CCC3=Cc4c(cnn4-c4ccc(F)cc4)C[C@]3(C(=O)c3cc(C(F)(F)F)ccn3)C2)cn1]

The title compound was prepared ((4aR,6R)-4a-(((tert-butyldimethylsilyl)oxy)methyl)-1-(4-fluorophenyl)-4,4a,5,6,7,8-hexahydro-1H-benzo[f]indazol-6-yl)methanol (118f-2) by procedures similar to those described in Example 118, Steps G, H, land J. ¹H NMR (400 MHz, Chloroform-d) n 8.79 (1H, d, J=5.2 Hz), 8.06 (1H, m), 7.84 (1H, s), 7.76 (1H, d, J=0.8 Hz), 7.65 (1H, ddd, J=5.2, 1.8, 0.6 Hz), 7.46-7.43 (2H, m), 7.30 (1H, s), 7.20-7.16 (2H, m), 6.37 (1H, d, J=1.2 Hz), 3.96 (3H, s), 3.86 (1H, d, J=16.8 Hz), 3.41 (1H, dt, J=13.6, 2.8 Hz), 3.01-3.0 (2H, m), 2.84 (1H, d, J=16.8 Hz), 2.46-2.41 (1H, m), 2.31-2.13 (2H, m), 2.03-2.00 (1H, m), 1.36-1.25 (2H, m); m/z (ESI, +ve ion)=600.1 [M+H]⁺. The C₆ stereochemistry of 119 is randomly assigned.

### Example 120. ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone (120)

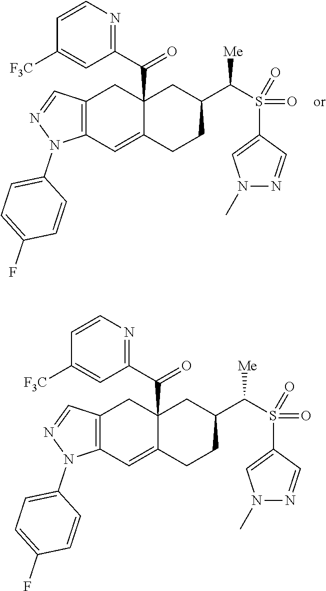 [SMILES: C[C@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; C[C@@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1]

### Step A: (4aR,6S)-4a-(((tert-Butyldimethylsilyl)oxy)methyl)-1-(4-fluorophenyl)-4,4a,5,6,7,8-hexahydro-1H-benzo[f]indazole-6-carbaldehyde (120a)

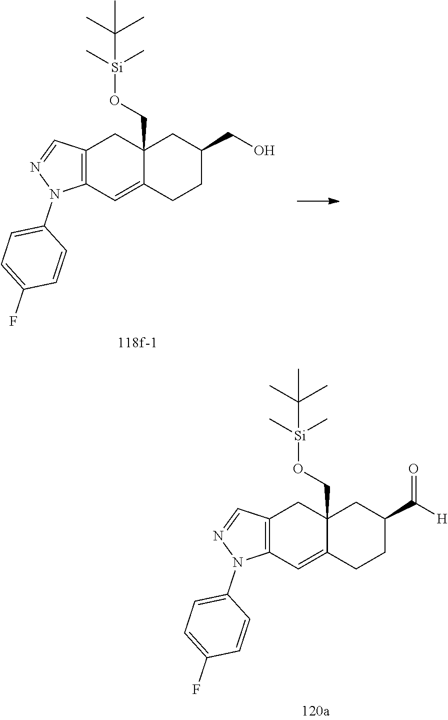 [SMILES: CC(C)(C)[Si](C)(C)OC[C@]12Cc3cnn(-c4ccc(F)cc4)c3C=C1CC[C@H](CO)C2 ; CC(C)(C)[Si](C)(C)OC[C@]12Cc3cnn(-c4ccc(F)cc4)c3C=C1CC[C@H](C=O)C2]

To a stirred solution of ((4aR,6S)-4a-(((tert-butyldimethylsilyl)oxy)methyl)-1-(4-fluorophenyl)-4,4a,5,6,7,8-hexahydro-1H-benzo[f]indazol-6-yl)methanol (118f-1) (267 mg, 0.60 mmol) in DCM (9.5 mL) was added Dess-Martin periodinane (269 mg, 0.63 mmol) at rt. After the reaction mixture was stirred for 30 min, it was quenched (sat. aq. NaHCO₃ and 10% aq. Na₂S₂O₃). The solution was stirred at rt for another 15 min and extracted (DCM). The organic layer was washed (brine), dried (Na₂SO₄), and concentrated under reduced pressure. Purification of the residue by silica gel column chromatography (24 g SiO₂, 5% to 30% EtOAc/hexanes, gradient elution) provided (4aR,6S)-4a-(((tert-butyldimethylsilyl)oxy)methyl)-1-(4-fluorophenyl)-4,4a,5,6,7,8-hexahydro-1H-benzo[f]indazole-6-carbaldehyde (120a) (235 mg, 88%) as a colorless gum. m/z (ESI, +ve ion)=441.3 [M+H].

### Step B: 1-((4aR,6S)-4a-(((tert-Butyldimethylsilyl)oxy)methyl)-1-(4-fluorophenyl)-4,4a,5,6,7,8-hexahydro-1H-benzo[f]indazol-6-yl)ethan-1-ol (120b)

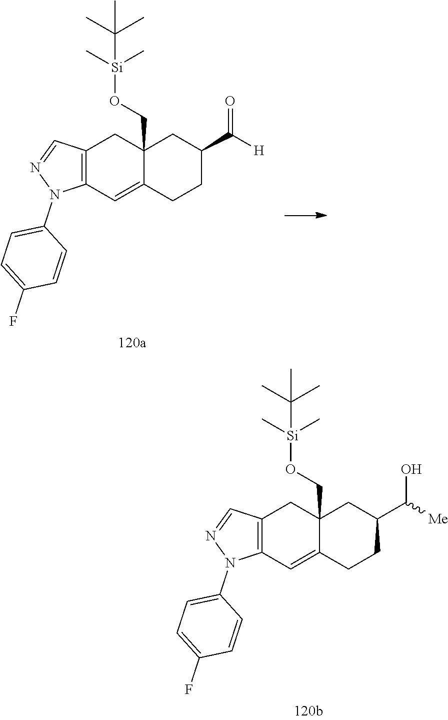 [SMILES: CC(C)(C)[Si](C)(C)OC[C@]12Cc3cnn(-c4ccc(F)cc4)c3C=C1CC[C@H](C=O)C2 ; CC(O)[C@H]1CCC2=Cc3c(cnn3-c3ccc(F)cc3)C[C@]2(CO[Si](C)(C)C(C)(C)C)C1]

To a stirred solution of (4aR,6S)-4a-(((tert-butyldimethylsilyl)oxy)methyl)-1-(4-fluorophenyl)-4,4a,5,6,7,8-hexahydro-1H-benzo[f]indazole-6-carbaldehyde (120a) (191 mg, 0.434 mmol) in THF (4 mL) at 0° C. was added methylmagnesium bromide solution (3.0 M in diethyl ether, 0.51 mL, 1.52 mmol) dropwise at 0° C. After the mixture was allowed to warm to rt and stirred for 1 h, it was quenched (sat. NH₄Cl aq.) and extracted (EtOAc). The organic layer was washed (brine), dried (Na₂SO₄), and concentrated under reduced pressure. Purification of the residue by silica gel column chromatography (40 g SiO₂, 5% to 20% acetone/hexanes, gradient elution) provided 1-((4aR,6S)-4a-(((tert-butyldimethylsilyl)oxy)methyl)-1-(4-fluorophenyl)-4,4a,5,6,7,8-hexahydro-1H-benzo[f]indazol-6-yl)ethan-1-ol (120b) (127 mg, 64%) as a white foamy solid. m/z (ESI, +ve ion)=457.3 [M+H]⁺.

### Step C: ((4aR,6S)-1-(4-Fluorophenyl)-6-(1-((1-methyl-1H-pyrazol-4-yl)thio)ethyl)-1,4,5,6,7,8-hexahydro-4aH-benzo[f]indazol-4a-yl)methanol (120c)

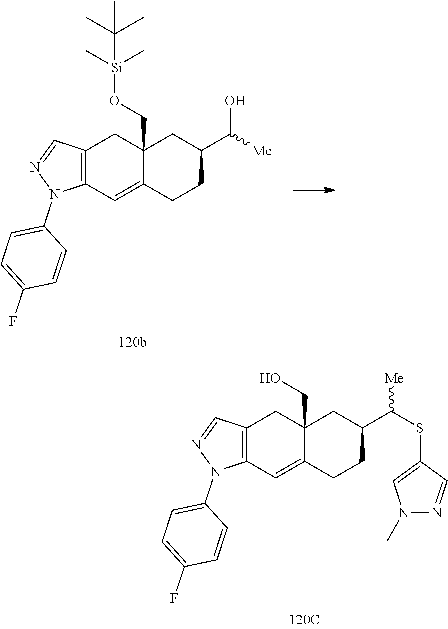

The title compound was prepared from 1-((4aR,6S)-4a-(((tert-butyldimethylsilyl)oxy)methyl)-1-(4-fluorophenyl)-4,4a,5,6,7,8-hexahydro-1H-benzo[f]indazol-6-yl)ethan-1-ol (120b) by procedures similar to those described in Example 118, Steps G, H, and I. m/z (ESI, +ve ion)=439.3 [M+H]⁺

### STEP D: ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (120d-1) and ((4aR,6S)-1-(4-fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (120d-2)

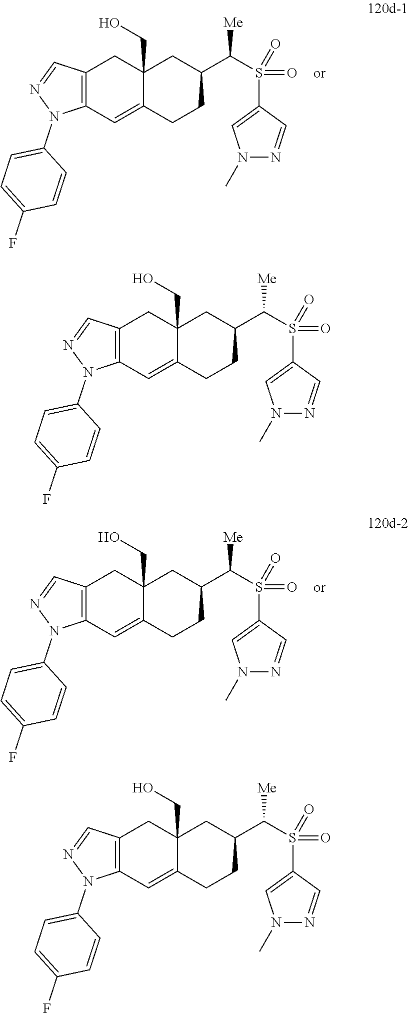 [SMILES: C[C@H]([C@H]1CCC2=Cc3c(cnn3-c3ccc(F)cc3)C[C@]2(CO)C1)S(=O)(=O)c1cnn(C)c1 ; C[C@@H]([C@H]1CCC2=Cc3c(cnn3-c3ccc(F)cc3)C[C@]2(CO)C1)S(=O)(=O)c1cnn(C)c1 ; C[C@H]([C@H]1CCC2=Cc3c(cnn3-c3ccc(F)cc3)C[C@]2(CO)C1)S(=O)(=O)c1cnn(C)c1 ; C[C@@H]([C@H]1CCC2=Cc3c(cnn3-c3ccc(F)cc3)C[C@]2(CO)C1)S(=O)(=O)c1cnn(C)c1]

To a stirred solution of ((4aR,6S)-1-(4-fluorophenyl)-6-(1-((1-methyl-1H-pyrazol-4-yl)thio)ethyl)-1,4,5,6,7,8-hexahydro-4aH-benzo[f]indazol-4a-yl)methanol (120c) (101 mg, 0.23 mmol) in MeOH (0.7 mL)/water (0.7 mL)/THF (1.4 mL) was added oxone (186 mg, 1.22 mmol) in one portion at rt. After the mixture was heated at 40° C. for 1 h and cooled down to 0° C., it was quenched (10% aq. Na₂S₂O₃, sat. aq. NaHCO₃) and extracted (EtOAc). The organic layer was washed (brine), dried (Na₂SO₄), and concentrated under reduced pressure. Purification of the residue by silica gel column chromatography (12 g SiO₂, 0% to 15% MeOH/EtOAc, gradient elution) provided the title compound (120d-1) (first eluting isomer, 40.4 mg, 35%) and (120d-2) (second eluting isomer, 53.2 mg, 46%). m/z (ESI, +ve ion) 471.3 [M+H]⁺.

### Step G: ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone (120)

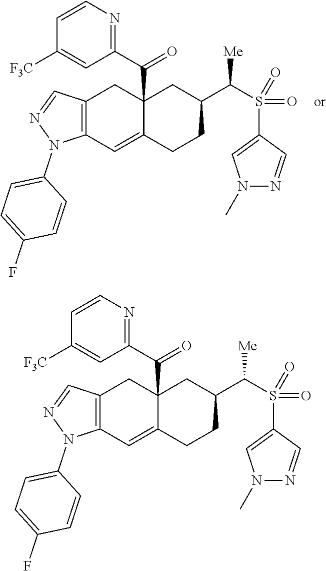 [SMILES: C[C@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; C[C@@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1]

The title compound was prepared from ((4aR,6S)-1-(4-fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (120d-1) by procedures similar to those described in Example 3, Steps D, E, and F. ¹H NMR (400 MHz, Chloroform-d) δ 8.89 (1H, d, J=5.2 Hz), 8.00 (1H, m), 7.84 (2H, m), 7.67-7.65 (1H, m), 7.45-7.42 (2H, m), 7.27 (1H, s), 7.18-7.14 (2H, m), 6.47 (1H, m), 4.01 (1H, d, J=15.2 Hz), 3.99 (3H, s), 3.18 (1H, d, J=16.8 Hz), 3.02-2.96 (1H, m), 2.89-2.81 (2H, m), 2.57-2.45 (2H, m), 1.95 (1H, t, J=13.4 Hz), 1.75-1.61 (2H, m), 1.32 (3H, d, J=7.2 Hz); m/z (ESI, +ve ion)=614.2 [M+H]⁺

### Example 121: ((4aR,6S)-1-(4-Fluorophenyl)-6-((R)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)(4-(trifluoromethyl)pyridin-2-yl)methanone (121)

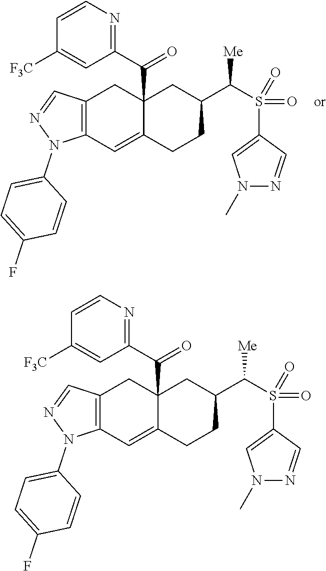 [SMILES: C[C@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; C[C@@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1]

The title compound was prepared from ((4aR,6S)-1-(4-fluorophenyl)-6-((R)-1-((1-methyl-1H-pyrazol-4-yl)sulfonyl)ethyl)-1,4,5,6,7,8-hexahydro-4aH-benzo[f]indazol-4a-yl)methanol or ((4aR,6S)-1-(4-fluorophenyl)-6-((S)-1-((1-methyl-1H-pyrazol-4-yl)sulfonyl)ethyl)-1,4,5,6,7,8-hexahydro-4aH-benzo[f]indazol-4a-yl)methanol (120d-2) by procedures similar to those described in Example 3, Steps D, E, and F.

¹H NMR (400 MHz, Chloroform-d) δ 8.84 (1H, d, J=4.8 Hz), 8.02 (1H, m), 7.82 (1H, s), 7.80 (1H, d, J=0.8 Hz), 7.66 (1H, ddd, J=5.2, 2.0, 0.5 Hz), 7.45-7.42 (2H, m), 7.26 (1H, s), 7.18-7.14 (2H, m), 6.47 (1H, s), 3.97 (3H, s), 3.93 (1H, d, J=16.4 Hz), 3.13-3.09 (2H, m), 2.84-2.78 (1H, m), 2.53-2.45 (3H, m), 2.19 (1H, t, J=13.2 Hz), 1.88-1.75 (2H, m), 1.31 (3H, d, J=6.8 Hz); m/z (ESI, +ve ion)=614.2 [M+H]⁺

### Example 122. (4aR,6S)-1-(4-Fluorophenyl)-N-methyl-N-(1-methyl-1H-pyrazol-4-yl)-4a-(4-(trifluoromethyl)picolinoyl)-4,4a,5,6,7,8-hexahydro-1H-benzo[f]indazole-6-carboxamide (122)

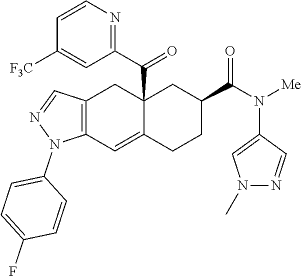 [SMILES: CN(C(=O)[C@H]1CCC2=Cc3c(cnn3-c3ccc(F)cc3)C[C@]2(C(=O)c2cc(C(F)(F)F)ccn2)C1)c1cnn(C)c1]

### Step A: (4aR)-4a-(((tert-Butyldimethylsilyl)oxy)methyl)-1-(4-fluorophenyl)-4,4a,5,6,7,8-hexahydro-1H-benzo[f]indazole-6-carboxylic acid (122a)

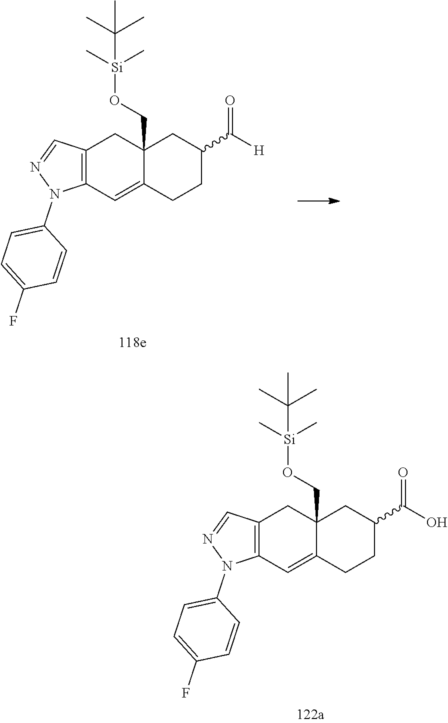 [SMILES: CC(C)(C)[Si](C)(C)OC[C@]12Cc3cnn(-c4ccc(F)cc4)c3C=C1CCC(C=O)C2 ; CC(C)(C)[Si](C)(C)OC[C@]12Cc3cnn(-c4ccc(F)cc4)c3C=C1CCC(C(=O)O)C2]

To a stirred solution of (4aR)-4a-(((tert-butyldimethylsilyl)oxy)methyl)-1-(4-fluorophenyl)-4,4a,5,6,7,8-hexahydro-1H-benzo[f]indazole-6-carbaldehyde (118e) (600 mg, 1.36 mmol) in DMF (7.0 mL) was added oxone (829 mg, 5.45 mmol) at 0° C. After the mixture was allowed to warm to rt and stirred at rt for 75 min, it was quenched (sat. aq. NaHCO₃, 10% aq. Na₂S₂O₃) and extracted (EtOAc). The organic layers were washed (brine), dried (Na₂SO₄), and concentrated under reduced pressure. Purification of the residue by silica gel column chromatography (40 g SiO₂, 20% to 40% EtOAc/hexanes, gradient elution) provided (4aR)-4a-(((tert-butyldimethylsilyl)oxy)methyl)-1-(4-fluorophenyl)-4,4a,5,6,7,8-hexahydro-1H-benzo[f]indazole-6-carboxylic acid (122a) as a diastereomeric mixture (cis:trans=1.9:1.0) (318 mg, 51%). m/z (ESI, +ve ion)=457.3 [M+H]⁺.

### Step B: (4aR,6R)-4a-(((tert-butyldimethylsilyl)oxy)methyl)-1-(4-fluorophenyl)-N-(1-methyl-1H-pyrazol-4-yl)-4,4a,5,6,7,8-hexahydro-1H-benzo[f]indazole-6-carboxamide (122b)

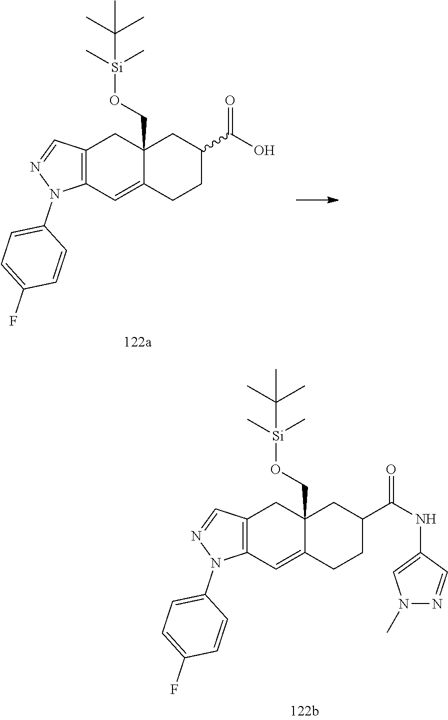 [SMILES: CC(C)(C)[Si](C)(C)OC[C@]12Cc3cnn(-c4ccc(F)cc4)c3C=C1CCC(C(=O)O)C2 ; Cn1cc(NC(=O)C2CCC3=Cc4c(cnn4-c4ccc(F)cc4)C[C@]3(CO[Si](C)(C)C(C)(C)C)C2)cn1]

To stirred solution of (4aR)-4a-(((tert-butyldimethylsilyl)oxy)methyl)-1-(4-fluorophenyl)-4,4a,5,6,7,8-hexahydro-1H-benzo[f]indazole-6-carboxylic acid (122a) (318 mg, 0.70 mmol) and 1-methyl-1H-pyrazol-4-amine (94.7 mg, 0.98 mmol) in pyridine (2.1 mL) was added 1-(3-dimethylaminopropyl)-3-ethylcarbodiimide hydrochloride (200 mg, 1.05 mmol) at rt. After the mixture was stirred at rt overnight and diluted with AcOEt, the organic layer was washed (water, 10% citric acid, sat. aq. NaHCO₃, and brine, respectively), dried (Na₂SO₄, and concentrated under reduced pressure. Purification of the residue by silica gel column chromatography (40 g SiO₂, 1% to 4% MeOH/DCM, gradient elution) provided the title compound (122b) (second eluting isomer, 215 mg, 58%) as an orange foamy solid. m/z (ESI, +ve ion)=536.3 [M+H]⁺. The C₆ stereochemistry of 122b is randomly assigned.

### Step C: (4aR,6S)-4a-(((tert-Butyldimethylsilyl)oxy)methyl)-1-(4-fluorophenyl)-N-methyl-N-(1-methyl-1H-pyrazol-4-yl)-4,4a,5,6,7,8-hexahydro-1H-benzo[f]indazole-6-carboxamide (122c)

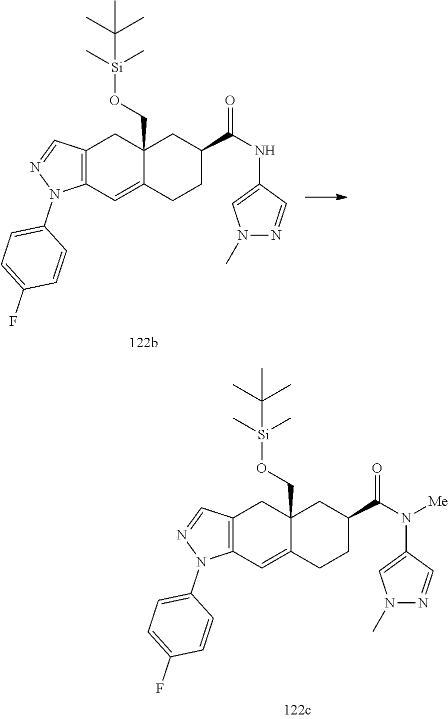 [SMILES: Cn1cc(NC(=O)[C@H]2CCC3=Cc4c(cnn4-c4ccc(F)cc4)C[C@]3(CO[Si](C)(C)C(C)(C)C)C2)cn1 ; CN(C(=O)[C@H]1CCC2=Cc3c(cnn3-c3ccc(F)cc3)C[C@]2(CO[Si](C)(C)C(C)(C)C)C1)c1cnn(C)c1]

To a stirred solution of (4aR,6S)-4a-(((tert-butyldimethylsilyl)oxy)methyl)-1-(4-fluorophenyl)-N-(1-methyl-1H-pyrazol-4-yl)-4,4a,5,6,7,8-hexahydro-1H-benzo[f]indazole-6-carboxamide (122b) (215 mg, 0.40 mmol) in DMF (2.5 mL) was added cesium carbonate (588 mg, 1.81 mmol) at 0° C. The mixture was allowed to warm to rt and stirred for 30 min. Iodomethane (87 L) was added and the resulting mixture was heated at 35° C. for 5 h. After the reaction was cooled down to rt, it was quenched (water) and extracted (EtOAc). The organic layer was washed (brine), dried (Na₂SO₄, and concentrated under reduced pressure. Purification of the residue by silica gel column chromatography (40 g SiO₂, 15% to 30% EtOAc/hexanes, gradient elution) provided (4aR,6S)-4a-(((tert-butyldimethylsilyl)oxy)methyl)-1-(4-fluorophenyl)-N-methyl-N-(1-methyl-1H-pyrazol-4-yl)-4,4a,5,6,7,8-hexahydro-1H-benzo[f]indazole-6-carboxamide (122c) (173 mg, 78%) as a white foamy solid. m/z (ESI, +ve ion)=550.3 [M+H]⁺.

### Step D: (4aR,6S)-1-(4-Fluorophenyl)-N-methyl-N-(1-methyl-1H-pyrazol-4-yl)-4a-(4-(trifluoromethyl)picolinoyl)-4,4a,5,6,7,8-hexahydro-1H-benzo[f]indazole-6-carboxamide (122)

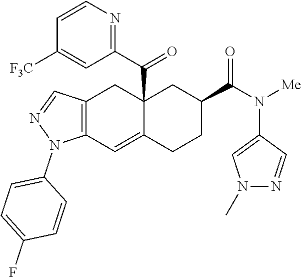 [SMILES: CN(C(=O)[C@H]1CCC2=Cc3c(cnn3-c3ccc(F)cc3)C[C@]2(C(=O)c2cc(C(F)(F)F)ccn2)C1)c1cnn(C)c1]

The title compound was prepared from 4aR,6S)-4a-(((tert-butyldimethylsilyl)oxy)methyl)-1-(4-fluorophenyl)-N-methyl-N-(1-methyl-1H-pyrazol-4-yl)-4,4a,5,6,7,8-hexahydro-1H-benzo[f]indazole-6-carboxamide (122c) by procedures similar to those described in Example 114, Steps D and E. Two rotamers were observed in the ¹H NMR of 122 at rt. H NMR of major rotamer (400 MHz, Chloroform-d) δ 8.84 (1H, d, J=4.8 Hz), 8.08 (1H, m), 7.65-7.63 (1H, m), 7.45-7.40 (3H, m), 7.38 (1H, s), 7.23 (1H, s), 7.17-7.12 (2H, m), 6.42 (1H, d, J=1.6 Hz), 3.97 (1H, d, J=16.4 Hz), 3.92 (3H, s), 3.12 (3H, s), 3.00-2.92 (1H, m), 2.65-2.44 (4H, m), 2.38-2.33 (1H, m), 2.07-1.99 (1H, m), 1.60-1.56 (1H, m); m/z (ESI, +ve ion)=579.3 [M+H]⁺. The C₆ stereochemistry of 122 is randomly assigned.

Examples 123-414: Examples 123-414 were prepared by similar procedures as described in Examples 1-7 and 111-122.

| Ex. | Name | Structures | MS (ESI) [M + H]⁺ |
|---|---|---|---|
| 123 | (5-cyclopropylpyridin-2- yl)((4aS,6S)-6-((2-ethyl-2H- 1,2,3-triazol-4-yl)sulfonyl)-1- (4-fluorophenyl)-1,4,5,6,7,8- hexahydro-4aH- benzo[f]indazol-4a- yl)methanone | 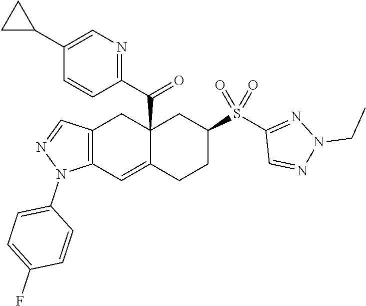 [SMILES: CCn1ncc(S(=O)(=O)[C@H]2CCC3=Cc4c(cnn4-c4ccc(F)cc4)C[C@]3(C(=O)c3ccc(C4CC4)cn3)C2)n1] | 573.2 |
| 124 | N-((4aS,6S)-4a-(5- cyclopropylpicolinoyl)-1-(4- fluorophenyl)-4,4a,5,6,7,8- hexahydro-1H- benzo[f]indazol-6-yl)-4-fluoro- N-methylbenzenesulfonamide | 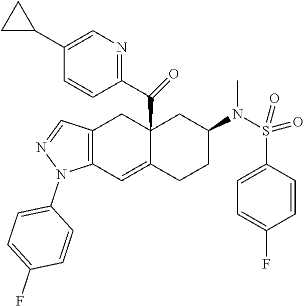 [SMILES: CN([C@H]1CCC2=Cc3c(cnn3-c3ccc(F)cc3)C[C@]2(C(=O)c2ccc(C3CC3)cn2)C1)S(=O)(=O)c1ccc(F)cc1] | 601.3 |
| 125 | ((4aS,6S)-6-fluoro-1-(4- fluorophenyl)-6- (phenylsulfonyl)-1,4,5,6,7,8- hexahydro-4aH- benzo[f]indazol-4a- yl)(pyridin-2-yl)methanone | 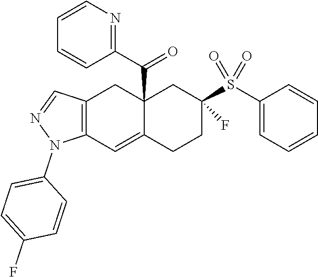 [SMILES: O=C(c1ccccn1)[C@]12Cc3cnn(-c4ccc(F)cc4)c3C=C1CC[C@](F)(S(=O)(=O)c1ccccc1)C2] | 532.1 |
| 126 | 2-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methyl-2H-1,2,3-triazole-4- sulfonamide | 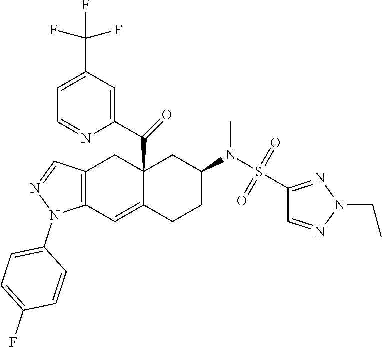 [SMILES: CCn1ncc(S(=O)(=O)N(C)[C@H]2CCC3=Cc4c(cnn4-c4ccc(F)cc4)C[C@]3(C(=O)c3cc(C(F)(F)F)ccn3)C2)n1] | 630.2 |
| 127 | 4-chloro-N-ethyl-N-((4aS,6S)- 1-(4-fluorophenyl)-4a- (thiazole-2-carbonyl)- 4,4a,5,6,7,8-hexahydro-1H- benzo[f]indazol-6- yl)benzenesulfonamide | 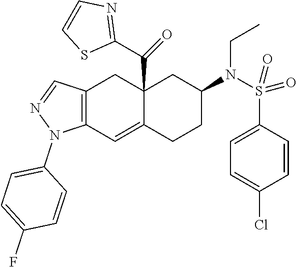 [SMILES: CCN([C@H]1CCC2=Cc3c(cnn3-c3ccc(F)cc3)C[C@]2(C(=O)c2nccs2)C1)S(=O)(=O)c1ccc(Cl)cc1] | 597.2 |
| 128 | N-ethyl-4-fluoro-N-((4aS,6S)- 1-(4-fluorophenyl)-4a- (thiazole-2-carbonyl)- 4,4a,5,6,7,8-hexahydro-1H- benzo[f]indazol-6- yl)benzenesulfonamide | 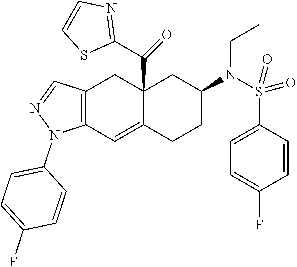 [SMILES: CCN([C@H]1CCC2=Cc3c(cnn3-c3ccc(F)cc3)C[C@]2(C(=O)c2nccs2)C1)S(=O)(=O)c1ccc(F)cc1] | 581.2 |
| 129 | 2-ethyl-N-((4aS,6S)-1-(4- fluorophenyl)-4a-(5- (trifluoromethyl)picolinoyl)- 4,4a,5,6,7,8-hexahydro-1H- benzo[f]indazol-6-yl)-N- methyl-2H-1,2,3-triazole-4- sulfonamide | 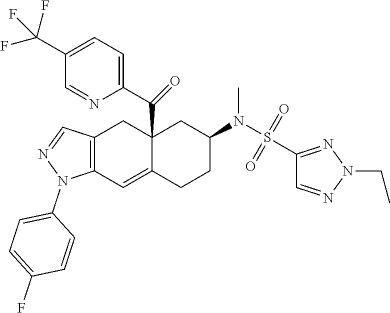 [SMILES: CCn1ncc(S(=O)(=O)N(C)[C@H]2CCC3=Cc4c(cnn4-c4ccc(F)cc4)C[C@]3(C(=O)c3ccc(C(F)(F)F)cn3)C2)n1] | 630.2 |
| 130 | N-((4aS,6S)-4a-(5- cyclopropylpicolinoyl)-1-(4- fluorophenyl)-4,4a,5,6,7,8- hexahydro-1H- benzo[f]indazol-6-yl)-2-ethyl- N-methyl-2H-1,2,3-triazole-4- sulfonamide | 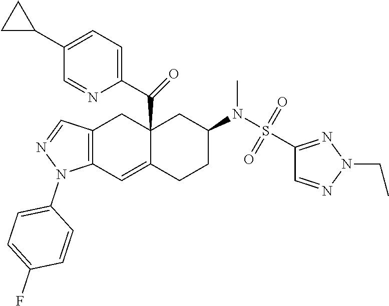 [SMILES: CCn1ncc(S(=O)(=O)N(C)[C@H]2CCC3=Cc4c(cnn4-c4ccc(F)cc4)C[C@]3(C(=O)c3ccc(C4CC4)cn3)C2)n1] | 602.3 |
| 131 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N,1- dimethyl-1H-pyrazole-4- sulfonamide | 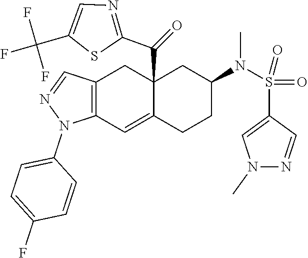 [SMILES: CN([C@H]1CCC2=Cc3c(cnn3-c3ccc(F)cc3)C[C@]2(C(=O)c2ncc(C(F)(F)F)s2)C1)S(=O)(=O)c1cnn(C)c1] | 621.1 |
| 132 | N,1-diethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1H- pyrazole-4-sulfonamide | 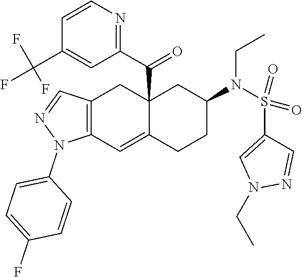 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cnn(CC)c1] | 643.3 |
| 133 | N-((4aS,6S)-1-(4- fluorophenyl)-4a-(thiazole-2- carbonyl)-4,4a,5,6,7,8- hexahydro-1H- benzo[f]indazol-6-yl)-N,1- dimethyl-1H-pyrazole-4- sulfonamide | 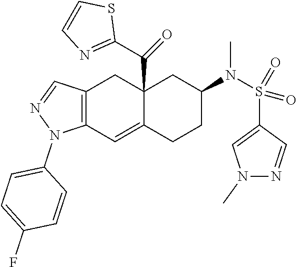 [SMILES: CN([C@H]1CCC2=Cc3c(cnn3-c3ccc(F)cc3)C[C@]2(C(=O)c2nccs2)C1)S(=O)(=O)c1cnn(C)c1] | 553.1 |
| 134 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N,1- dimethyl-1H-pyrazole-4- sulfonamide | 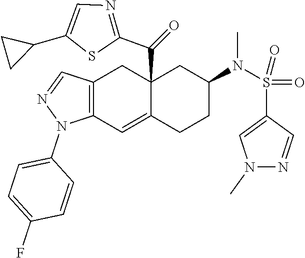 [SMILES: CN([C@H]1CCC2=Cc3c(cnn3-c3ccc(F)cc3)C[C@]2(C(=O)c2ncc(C3CC3)s2)C1)S(=O)(=O)c1cnn(C)c1] | 593.2 |
| 135 | N-((4aS,6S)-4a-(5- cyclopropylpicolinoyl)-1-(4- fluorophenyl)-4,4a,5,6,7,8- hexahydro-1H- benzo[f]indazol-6-yl)-N,1- dimethyl-1H-pyrazole-3- sulfonamide | 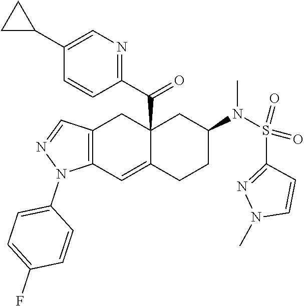 [SMILES: CN([C@H]1CCC2=Cc3c(cnn3-c3ccc(F)cc3)C[C@]2(C(=O)c2ccc(C3CC3)cn2)C1)S(=O)(=O)c1ccn(C)n1] | 587.3 |
| 136 | N-((4aS,6S)-4a-(5- cyclopropylpicolinoyl)-1-(4- fluorophenyl)-4,4a,5,6,7,8- hexahydro-1H- benzo[f]indazol-6-yl)-N-ethyl- 1-methyl-1H-pyrazole-4- sulfonamide | 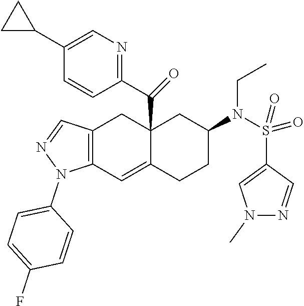 [SMILES: CCN([C@H]1CCC2=Cc3c(cnn3-c3ccc(F)cc3)C[C@]2(C(=O)c2ccc(C3CC3)cn2)C1)S(=O)(=O)c1cnn(C)c1] | 601.3 |
| 137 | (4aS,6S)-N,1-bis(4- fluorophenyl)-N-methyl-4a- picolinoyl-4,4a,5,6,7,8- hexahydro-1H- benzo[f]indazole-6- sulfonamide | 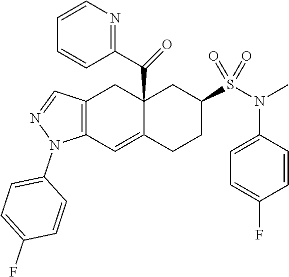 [SMILES: CN(c1ccc(F)cc1)S(=O)(=O)[C@H]1CCC2=Cc3c(cnn3-c3ccc(F)cc3)C[C@]2(C(=O)c2ccccn2)C1] | 561.2 |
| 138 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 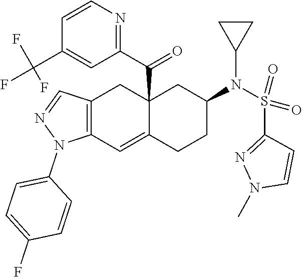 [SMILES: Cn1ccc(S(=O)(=O)N(C2CC2)[C@H]2CCC3=Cc4c(cnn4-c4ccc(F)cc4)C[C@]3(C(=O)c3cc(C(F)(F)F)ccn3)C2)n1] | 641.2 |
| 139 | N-((4aS,6S)-1-(4- fluorophenyl)-4a-(thiazole-2- carbonyl)-4,4a,5,6,7,8- hexahydro-1H- benzo[f]indazol-6-yl)-N,1- dimethyl-1H-pyrazole-3- sulfonamide | 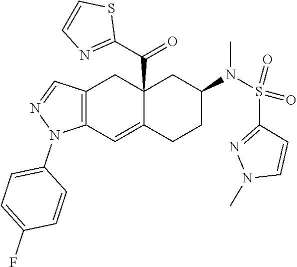 [SMILES: CN([C@H]1CCC2=Cc3c(cnn3-c3ccc(F)cc3)C[C@]2(C(=O)c2nccs2)C1)S(=O)(=O)c1ccn(C)n1] | 553.2 |
| 140 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N,1- dimethyl-1H-pyrazole-3- sulfonamide | 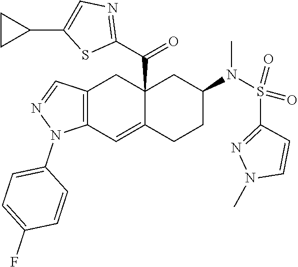 [SMILES: CN([C@H]1CCC2=Cc3c(cnn3-c3ccc(F)cc3)C[C@]2(C(=O)c2ncc(C3CC3)s2)C1)S(=O)(=O)c1ccn(C)n1] | 593.2 |
| 141 | N-cyclopropyl-N-((4aS,6S)- 4a-(5-cyclopropylpicolinoyl)- 1-(4-fluorophenyl)- 4,4a,5,6,7,8-hexahydro-1H- benzo[f]indazol-6-yl)-1- methyl-1H-pyrazole-4- sulfonamide | 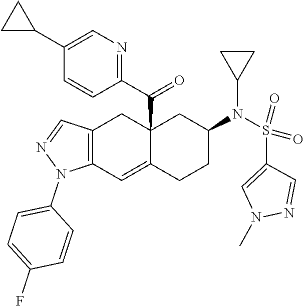 [SMILES: Cn1cc(S(=O)(=O)N(C2CC2)[C@H]2CCC3=Cc4c(cnn4-c4ccc(F)cc4)C[C@]3(C(=O)c3ccc(C4CC4)cn3)C2)cn1] | 613.3 |
| 142 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-2- methyl-2H-1,2,3-triazole-4- sulfonamide | 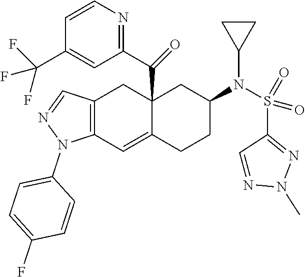 [SMILES: Cn1ncc(S(=O)(=O)N(C2CC2)[C@H]2CCC3=Cc4c(cnn4-c4ccc(F)cc4)C[C@]3(C(=O)c3cc(C(F)(F)F)ccn3)C2)n1] | 642.2 |
| 143 | 4-chloro-N-((4aS,6S)-1-(4- fluorophenyl)-4a-(thiazole-2- carbonyl)-4,4a,5,6,7,8- hexahydro-1H- benzo[f]indazol-6-yl)-N- methylbenzenesulfonamide | 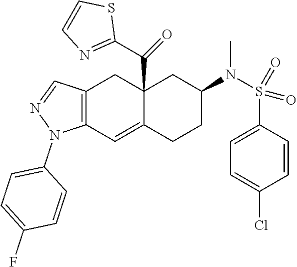 [SMILES: CN([C@H]1CCC2=Cc3c(cnn3-c3ccc(F)cc3)C[C@]2(C(=O)c2nccs2)C1)S(=O)(=O)c1ccc(Cl)cc1] | 583.1 |
| 144 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N,1- dimethyl-1H-pyrazole-3- sulfonamide | 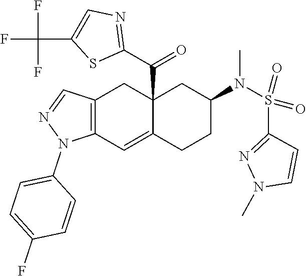 [SMILES: CN([C@H]1CCC2=Cc3c(cnn3-c3ccc(F)cc3)C[C@]2(C(=O)c2ncc(C(F)(F)F)s2)C1)S(=O)(=O)c1ccn(C)n1] | 621.2 |
| 145 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-2- methyl-2H-1,2,3-triazole-4- sulfonamide | 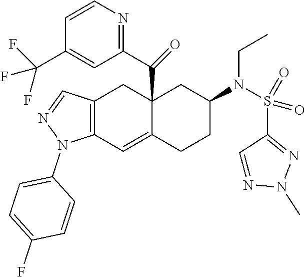 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cnn(C)n1] | 630.2 |
| 146 | 4-chloro-N-((4aS,6S)-4a-(5- cyclopropylpicolinoyl)-1-(4- fluorophenyl)-4,4a,5,6,7,8- hexahydro-1H- benzo[f]indazol-6-yl)-N- methylbenzenesulfonamide | 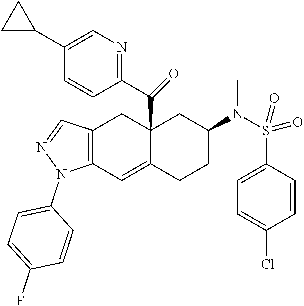 [SMILES: CN([C@H]1CCC2=Cc3c(cnn3-c3ccc(F)cc3)C[C@]2(C(=O)c2ccc(C3CC3)cn2)C1)S(=O)(=O)c1ccc(Cl)cc1] | 617.2 |
| 147 | N-cyclopropyl-N-((4aS,6S)-1- (4-fluorophenyl)-4a-(thiazole- 2-carbonyl)-4,4a,5,6,7,8- hexahydro-1H- benzo[f]indazol-6-yl)-1- methyl-1H-pyrazole-3- sulfonamide | 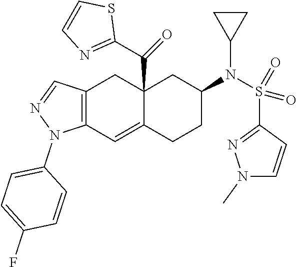 [SMILES: Cn1ccc(S(=O)(=O)N(C2CC2)[C@H]2CCC3=Cc4c(cnn4-c4ccc(F)cc4)C[C@]3(C(=O)c3nccs3)C2)n1] | 579.2 |
| 148 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N,2- dimethyl-2H-1,2,3-triazole-4- sulfonamide | 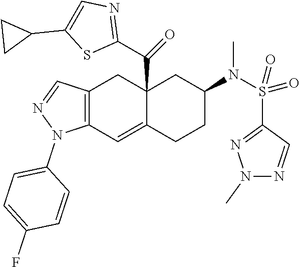 [SMILES: CN([C@H]1CCC2=Cc3c(cnn3-c3ccc(F)cc3)C[C@]2(C(=O)c2ncc(C3CC3)s2)C1)S(=O)(=O)c1cnn(C)n1] | 594.2 |
| 149 | N-((4aS,6S)-4a-(4- cyclopropylpicolinoyl)-1-(4- fluorophenyl)-4,4a,5,6,7,8- hexahydro-1H- benzo[f]indazol-6-yl)-N,2- dimethyl-2H-1,2,3-triazole-4- sulfonamide | 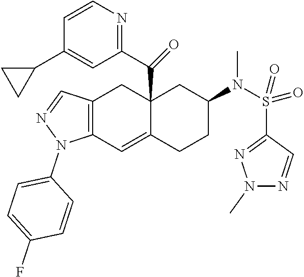 [SMILES: CN([C@H]1CCC2=Cc3c(cnn3-c3ccc(F)cc3)C[C@]2(C(=O)c2cc(C3CC3)ccn2)C1)S(=O)(=O)c1cnn(C)n1] | 588.2 |
| 150 | N-((4aS,6S)-4a-(4- cyclopropylthiazole-2- carbonyl)-1-(4-fluorophenyl)- 4,4a,5,6,7,8-hexahydro-1H- benzo[f]indazol-6-yl)-N,2- dimethyl-2H-1,2,3-triazole-4- sulfonamide | 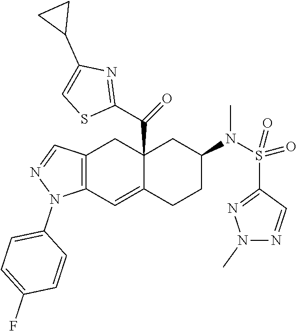 [SMILES: CN([C@H]1CCC2=Cc3c(cnn3-c3ccc(F)cc3)C[C@]2(C(=O)c2nc(C3CC3)cs2)C1)S(=O)(=O)c1cnn(C)n1] | 594.2 |
| 151 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N,2- dimethyl-2H-1,2,3-triazole-4- sulfonamide | 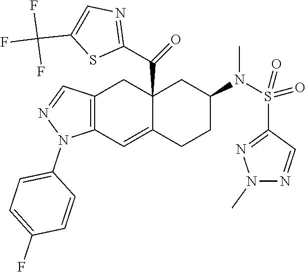 [SMILES: CN([C@H]1CCC2=Cc3c(cnn3-c3ccc(F)cc3)C[C@]2(C(=O)c2ncc(C(F)(F)F)s2)C1)S(=O)(=O)c1cnn(C)n1] | 622.1 |
| 152 | N-cyclopropyl-N-((4aS,6S)- 4a-(5-cyclopropylpicolinoyl)- 1-(4-fluorophenyl)- 4,4a,5,6,7,8-hexahydro-1H- benzo[f]indazol-6-yl)-1- methyl-1H-pyrazole-3- sulfonamide | 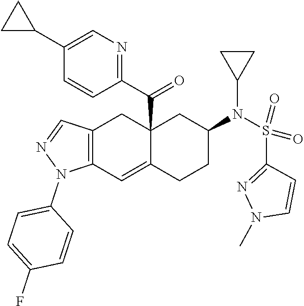 [SMILES: Cn1ccc(S(=O)(=O)N(C2CC2)[C@H]2CCC3=Cc4c(cnn4-c4ccc(F)cc4)C[C@]3(C(=O)c3ccc(C4CC4)cn3)C2)n1] | 613.3 |
| 153 | N-ethyl-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-pyrazole-4- sulfonamide | 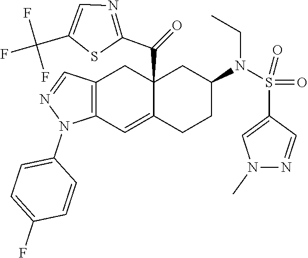 [SMILES: CCN([C@H]1CCC2=Cc3c(cnn3-c3ccc(F)cc3)C[C@]2(C(=O)c2ncc(C(F)(F)F)s2)C1)S(=O)(=O)c1cnn(C)c1] | 635.2 |
| 154 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N-ethyl- 1-methyl-1H-pyrazole-4- sulfonamide | 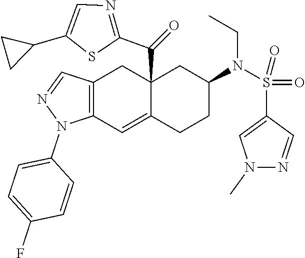 [SMILES: CCN([C@H]1CCC2=Cc3c(cnn3-c3ccc(F)cc3)C[C@]2(C(=O)c2ncc(C3CC3)s2)C1)S(=O)(=O)c1cnn(C)c1] | 607.2 |
| 155 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 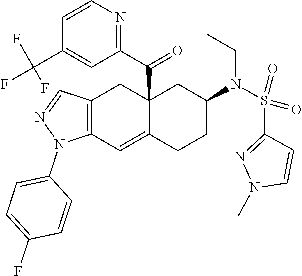 [SMILES: CCN([C@H]1CCC2=Cc3c(cnn3-c3ccc(F)cc3)C[C@]2(C(=O)c2cc(C(F)(F)F)ccn2)C1)S(=O)(=O)c1ccn(C)n1] | 629.1 |
| 156 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-pyrazole-5- sulfonamide | 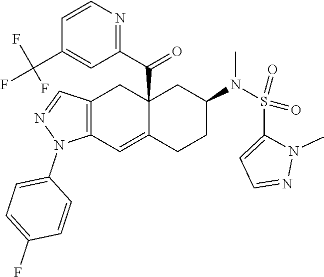 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccnn1C] | 615.2 |
| 157 | 1-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- methyl-1H-pyrazole-3- sulfonamide | 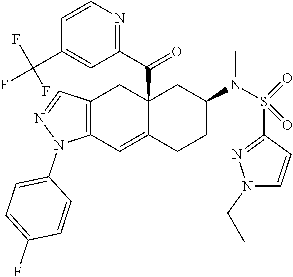 [SMILES: CCn1ccc(S(=O)(=O)N(C)[C@H]2CCC3=Cc4c(cnn4-c4ccc(F)cc4)C[C@]3(C(=O)c3cc(C(F)(F)F)ccn3)C2)n1] | 629.2 |
| 158 | N-cyclopropyl-1-ethyl-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1H- pyrazole-4-sulfonamide | 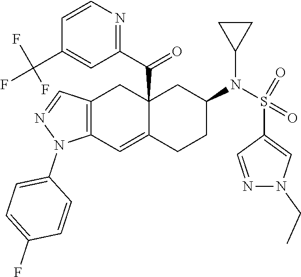 [SMILES: CCn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1] | 655.3 |
| 159 | N-cyclopropyl-4-fluoro-N- ((4aS,6S)-1-(4-fluorophenyl)- 4a-(thiazole-2-carbonyl)- 4,4a,5,6,7,8-hexahydro-1H- benzo[f]indazol-6- yl)benzenesulfonamide | 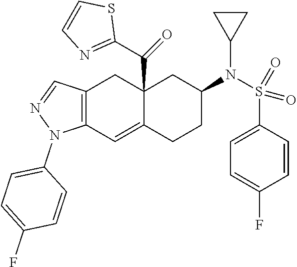 [SMILES: O=C(c1nccs1)[C@]12Cc3cnn(-c4ccc(F)cc4)c3C=C1CC[C@H](N(C1CC1)S(=O)(=O)c1ccc(F)cc1)C2] | 593.2 |
| 160 | N-cyclopropyl-N-((4aS,6S)- 4a-(5-cyclopropylthiazole-2- carbonyl)-1-(4-fluorophenyl)- 4,4a,5,6,7,8-hexahydro-1H- benzo[f]indazol-6-yl)-1- methyl-1H-pyrazole-3- sulfonamide | 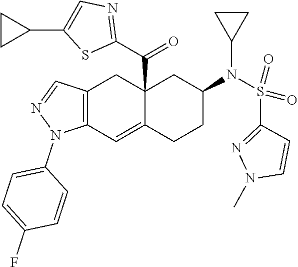 [SMILES: Cn1ccc(S(=O)(=O)N(C2CC2)[C@H]2CCC3=Cc4c(cnn4-c4ccc(F)cc4)C[C@]3(C(=O)c3ncc(C4CC4)s3)C2)n1] | 619.3 |
| 161 | N-((4aS,6S)-1-(4- fluorophenyl)-4a-(thiazole-2- carbonyl)-4,4a,5,6,7,8- hexahydro-1H- benzo[f]indazol-6-yl)-N,2- dimethyl-2H-1,2,3-triazole-4- sulfonamide | 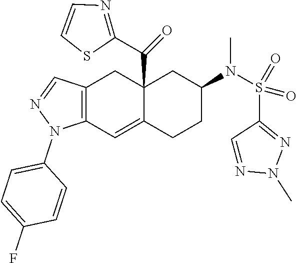 [SMILES: CN([C@H]1CCC2=Cc3c(cnn3-c3ccc(F)cc3)C[C@]2(C(=O)c2nccs2)C1)S(=O)(=O)c1cnn(C)n1] | 554.2 |
| 162 | 2-cyclopropyl-N-((4aS,6S)-1- (4-fluorophenyl)-4a-(thiazole- 2-carbonyl)-4,4a,5,6,7,8- hexahydro-1H- benzo[f]indazol-6-yl)-N- methyl-2H-1,2,3-triazole-4- sulfonamide | 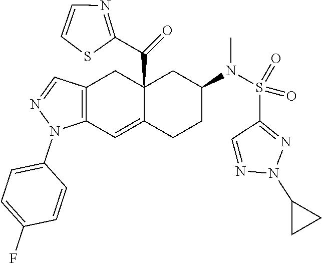 [SMILES: CN([C@H]1CCC2=Cc3c(cnn3-c3ccc(F)cc3)C[C@]2(C(=O)c2nccs2)C1)S(=O)(=O)c1cnn(C2CC2)n1] | 580.2 |
| 163 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-imidazole-4- sulfonamide | 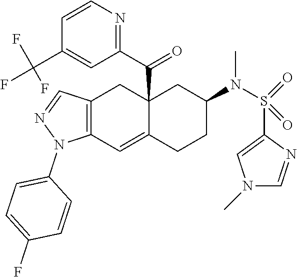 [SMILES: CN([C@H]1CCC2=Cc3c(cnn3-c3ccc(F)cc3)C[C@]2(C(=O)c2cc(C(F)(F)F)ccn2)C1)S(=O)(=O)c1cn(C)cn1] | 615.2 |
| 164 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-pyrazole-3- sulfonamide | 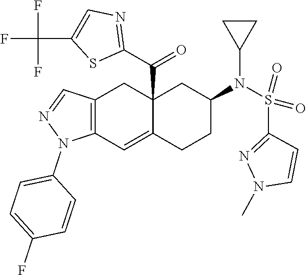 [SMILES: Cn1ccc(S(=O)(=O)N(C2CC2)[C@H]2CCC3=Cc4c(cnn4-c4ccc(F)cc4)C[C@]3(C(=O)c3ncc(C(F)(F)F)s3)C2)n1] | 647.2 |
| 165 | 1-(difluoromethyl)-N-ethyl-N- ((4aS,6S)-1-(4-fluorophenyl)- 4a-(thiazole-2-carbonyl)- 4,4a,5,6,7,8-hexahydro-1H- benzo[f]indazol-6-yl)-1H- pyrazole-4-sulfonamide | 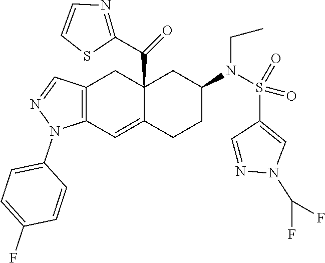 [SMILES: CCN([C@H]1CCC2=Cc3c(cnn3-c3ccc(F)cc3)C[C@]2(C(=O)c2nccs2)C1)S(=O)(=O)c1cnn(C(F)F)c1] | 603.2 |
| 166 | 1-(difluoromethyl)-N-ethyl-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1H- pyrazole-4-sulfonamide | 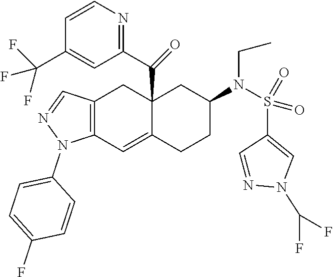 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cnn(C(F)F)c1] | 665.2 |
| 167 | N-ethyl-N-((4aS,6S)-1-(4- fluorophenyl)-4a-(thiazole-2- carbonyl)-4,4a,5,6,7,8- hexahydro-1H- benzo[f]indazol-6-yl)-1- methyl-1H-pyrazole-3- sulfonamide | 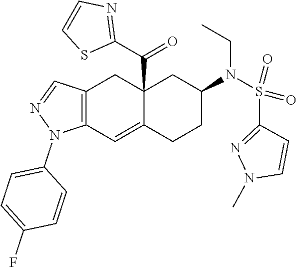 [SMILES: CCN([C@H]1CCC2=Cc3c(cnn3-c3ccc(F)cc3)C[C@]2(C(=O)c2nccs2)C1)S(=O)(=O)c1ccn(C)n1] | 567.1 |
| 168 | N-cyclopropyl-N-((4aS,6S)-1- (4-fluorophenyl)-4a-(thiazole- 2-carbonyl)-4,4a,5,6,7,8- hexahydro-1H- benzo[f]indazol-6-yl)-1- methyl-1H-pyrazole-4- sulfonamide | 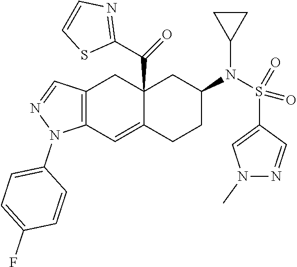 [SMILES: Cn1cc(S(=O)(=O)N(C2CC2)[C@H]2CCC3=Cc4c(cnn4-c4ccc(F)cc4)C[C@]3(C(=O)c3nccs3)C2)cn1] | 579.2 |
| 169 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-1,2,3-triazole-5- sulfonamide | 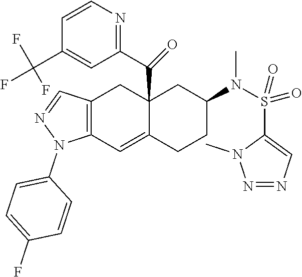 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnnn1C] | 616.3 |
| 170 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N,1- dimethyl-1H-1,2,3-triazole-4- sulfonamide | 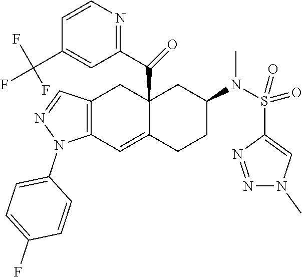 [SMILES: CN([C@H]1CCC2=Cc3c(cnn3-c3ccc(F)cc3)C[C@]2(C(=O)c2cc(C(F)(F)F)ccn2)C1)S(=O)(=O)c1cn(C)nn1] | 616.3 |
| 171 | N-cyclopropyl-N-((4aS,6S)- 4a-(5-cyclopropylthiazole-2- carbonyl)-1-(4-fluorophenyl)- 4,4a,5,6,7,8-hexahydro-1H- benzo[f]indazol-6-yl)-1- methyl-1H-pyrazole-4- sulfonamide | 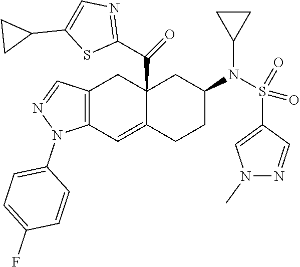 [SMILES: Cn1cc(S(=O)(=O)N(C2CC2)[C@H]2CCC3=Cc4c(cnn4-c4ccc(F)cc4)C[C@]3(C(=O)c3ncc(C4CC4)s3)C2)cn1] | 619.2 |
| 172 | N-(cyclopropylmethyl)-N- ((4aS,6S)-1-(4-fluorophenyl)- 4a-(thiazole-2-carbonyl)- 4,4a,5,6,7,8-hexahydro-1H- benzo[f]indazol-6-yl)-1- methyl-1H-pyrazole-3- sulfonamide | 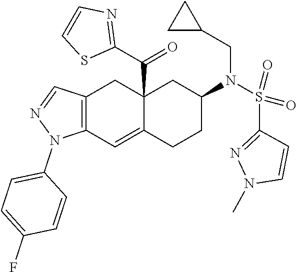 [SMILES: Cn1ccc(S(=O)(=O)N(CC2CC2)[C@H]2CCC3=Cc4c(cnn4-c4ccc(F)cc4)C[C@]3(C(=O)c3nccs3)C2)n1] | 593.2 |
| 173 | N-cyclopropyl-N-((4aS,6S)-1- (4-fluorophenyl)-4a-(thiazole- 2-carbonyl)-4,4a,5,6,7,8- hexahydro-1H- benzo[f]indazol-6-yl)-2- methyl-2H-1,2,3-triazole-4- sulfonamide | 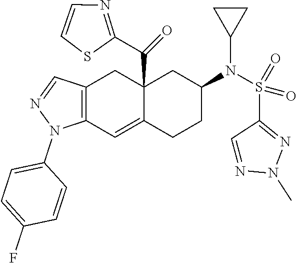 [SMILES: Cn1ncc(S(=O)(=O)N(C2CC2)[C@H]2CCC3=Cc4c(cnn4-c4ccc(F)cc4)C[C@]3(C(=O)c3nccs3)C2)n1] | 580.3 |
| 174 | N-cyclopropyl-N-((4aS,6S)- 4a-(5-cyclopropylthiazole-2- carbonyl)-1-(4-fluorophenyl)- 4,4a,5,6,7,8-hexahydro-1H- benzo[f]indazol-6-yl)-2- methyl-2H-1,2,3-triazole-4- sulfonamide | 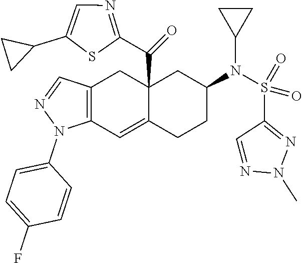 [SMILES: Cn1ncc(S(=O)(=O)N(C2CC2)[C@H]2CCC3=Cc4c(cnn4-c4ccc(F)cc4)C[C@]3(C(=O)c3ncc(C4CC4)s3)C2)n1] | 620.2 |
| 175 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N-ethyl- 1-methyl-1H-pyrazole-3- sulfonamide | 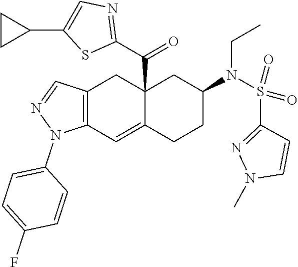 [SMILES: CCN([C@H]1CCC2=Cc3c(cnn3-c3ccc(F)cc3)C[C@]2(C(=O)c2ncc(C3CC3)s2)C1)S(=O)(=O)c1ccn(C)n1] | 607.3 |
| 176 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-pyrazole-4- sulfonamide | 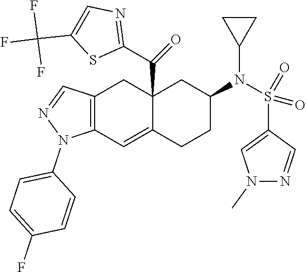 [SMILES: Cn1cc(S(=O)(=O)N(C2CC2)[C@H]2CCC3=Cc4c(cnn4-c4ccc(F)cc4)C[C@]3(C(=O)c3ncc(C(F)(F)F)s3)C2)cn1] | 647.2 |
| 177 | N-cyclopropyl-4-fluoro-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6- yl)benzenesulfonamide | 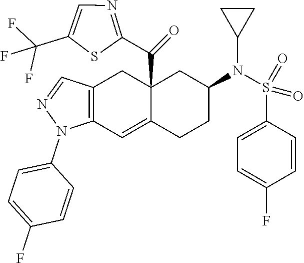 [SMILES: O=C(c1ncc(C(F)(F)F)s1)[C@]12Cc3cnn(-c4ccc(F)cc4)c3C=C1CC[C@H](N(C1CC1)S(=O)(=O)c1ccc(F)cc1)C2] | 661.2 |
| 178 | N-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-pyrazole-3- sulfonamide | 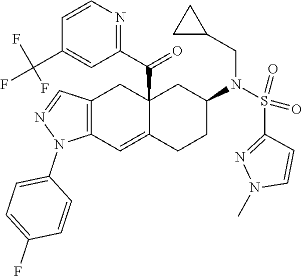 [SMILES: Cn1ccc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1] | 655.2 |
| 179 | N-(cyclopropylmethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 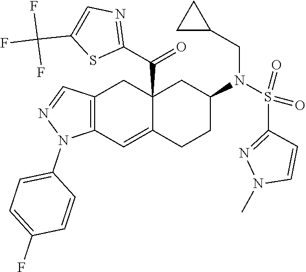 [SMILES: Cn1ccc(S(=O)(=O)N(CC2CC2)[C@H]2CCC3=Cc4c(cnn4-c4ccc(F)cc4)C[C@]3(C(=O)c3ncc(C(F)(F)F)s3)C2)n1] | 661.2 |
| 180 | N-cyclopropyl-1- (difluoromethyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1H- pyrazole-4-sulfonamide | 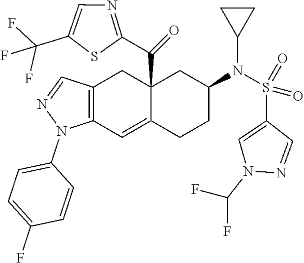 [SMILES: O=C(c1ncc(C(F)(F)F)s1)[C@]12Cc3cnn(-c4ccc(F)cc4)c3C=C1CC[C@H](N(C1CC1)S(=O)(=O)c1cnn(C(F)F)c1)C2] | 683.2 |
| 181 | N-(cyclopropylmethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-4- sulfonamide | 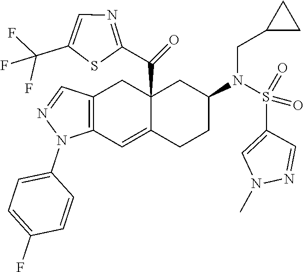 [SMILES: Cn1cc(S(=O)(=O)N(CC2CC2)[C@H]2CCC3=Cc4c(cnn4-c4ccc(F)cc4)C[C@]3(C(=O)c3ncc(C(F)(F)F)s3)C2)cn1] | 661.3 |
| 182 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H- pyrazole-3-sulfonamide | 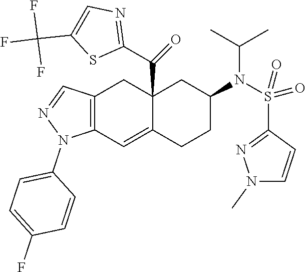 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccn(C)n1] | 649.3 |
| 183 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-2- methyl-2H-1,2,3-triazole-4- sulfonamide | 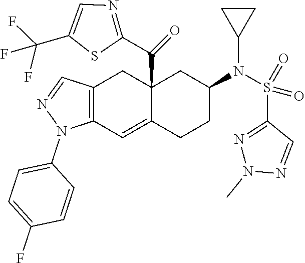 [SMILES: Cn1ncc(S(=O)(=O)N(C2CC2)[C@H]2CCC3=Cc4c(cnn4-c4ccc(F)cc4)C[C@]3(C(=O)c3ncc(C(F)(F)F)s3)C2)n1] | 648.2 |
| 184 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(2,2,2- trifluoroethyl)-1H-pyrazole-3- sulfonamide | 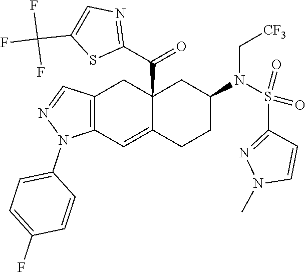 [SMILES: Cn1ccc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1] | 689.1 |
| 185 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N- isopropyl-1-methyl-1H- pyrazole-3-sulfonamide | 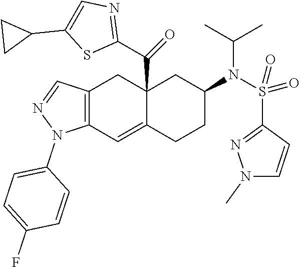 [SMILES: CC(C)N([C@H]1CCC2=Cc3c(cnn3-c3ccc(F)cc3)C[C@]2(C(=O)c2ncc(C3CC3)s2)C1)S(=O)(=O)c1ccn(C)n1] | 621.2 |
| 186 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-1-methyl-1H- pyrazole-3-sulfonamide | 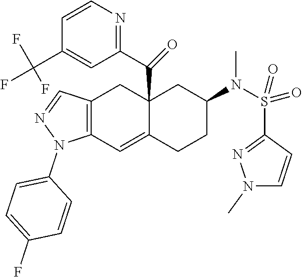 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccn(C)n1] | 643.3 |
| 187 | N-(cyclopropylmethyl)-N- ((4aS,6S)-4a-(5- cyclopropylthiazole-2- carbonyl)-1-(4-fluorophenyl)- 4,4a,5,6,7,8-hexahydro-1H- benzo[f]indazol-6-yl)-1- methyl-1H-pyrazole-3- sulfonamide | 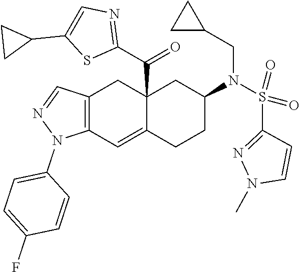 [SMILES: Cn1ccc(S(=O)(=O)N(CC2CC2)[C@H]2CCC3=Cc4c(cnn4-c4ccc(F)cc4)C[C@]3(C(=O)c3ncc(C4CC4)s3)C2)n1] | 633.3 |
| 188 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-pyrazole-4- carboxamide | 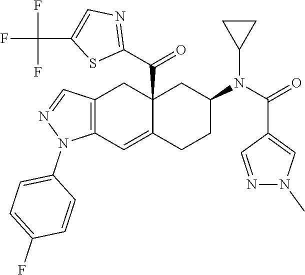 [SMILES: Cn1cc(C(=O)N(C2CC2)[C@H]2CCC3=Cc4c(cnn4-c4ccc(F)cc4)C[C@]3(C(=O)c3ncc(C(F)(F)F)s3)C2)cn1] | 611.3 |
| 189 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- hydroxy-2-methylpropyl)-1- methyl-1H-pyrazole-3- sulfonamide | 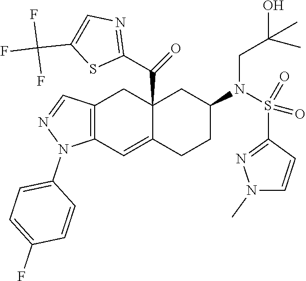 [SMILES: Cn1ccc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1] | 679.1 |
| 190 | ((4aS,6S)-1-(4-fluorophenyl)- 6-((1-methyl-1H-pyrazol-4- yl)sulfonyl)-1,4,5,6,7,8- hexahydro-4aH- benzo[f]indazol-4a-yl)(5- (trifluoromethyl)thiazol-2- yl)methanone | 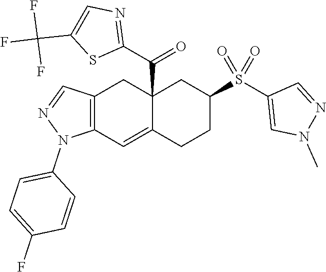 [SMILES: Cn1cc(S(=O)(=O)[C@H]2CCC3=Cc4c(cnn4-c4ccc(F)cc4)C[C@]3(C(=O)c3ncc(C(F)(F)F)s3)C2)cn1] | 592 |
| 191 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H- pyrazole-4-sulfonamide | 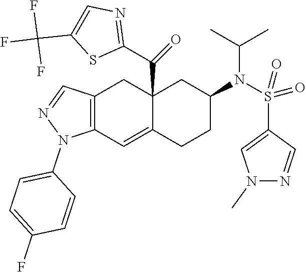 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)c1] | 649.3 |
| 192 | N-((4aS,6S)-4a-(5- cyclopropylthiazole-2- carbonyl)-1-(4-fluorophenyl)- 4,4a,5,6,7,8-hexahydro-1H- benzo[f]indazol-6-yl)-N- isopropyl-1-methyl-1H- pyrazole-4-sulfonamide | 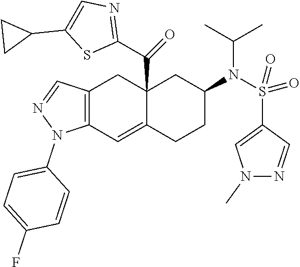 [SMILES: CC(C)N([C@H]1CCC2=Cc3c(cnn3-c3ccc(F)cc3)C[C@]2(C(=O)c2ncc(C3CC3)s2)C1)S(=O)(=O)c1cnn(C)c1] | 621.2 |
| 193 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-1-methyl-1H- pyrazole-4-sulfonamide | 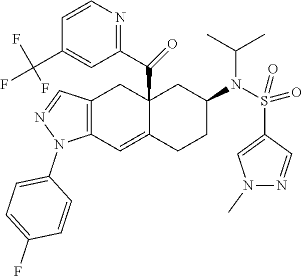 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)c1] | 643.3 |
| 194 | N-(cyclopropylmethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-2- methyl-2H-1,2,3-triazole-4- sulfonamide | 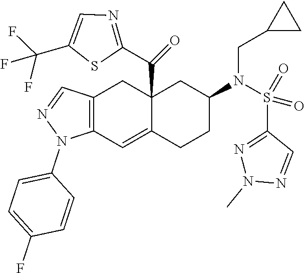 [SMILES: Cn1ncc(S(=O)(=O)N(CC2CC2)[C@H]2CCC3=Cc4c(cnn4-c4ccc(F)cc4)C[C@]3(C(=O)c3ncc(C(F)(F)F)s3)C2)n1] | 662.2 |
| 195 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N-(2- hydroxy-2-methylpropyl)-1- methyl-1H-pyrazole-3- sulfonamide | 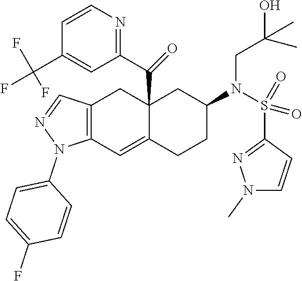 [SMILES: Cn1ccc(S(=O)(=O)N(CC(C)(C)O)[C@H]2CCC3=Cc4c(cnn4-c4ccc(F)cc4)C[C@]3(C(=O)c3cc(C(F)(F)F)ccn3)C2)n1] | 673.3 |
| 196 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(oxetan-3-yl)-1H- pyrazole-3-sulfonamide | 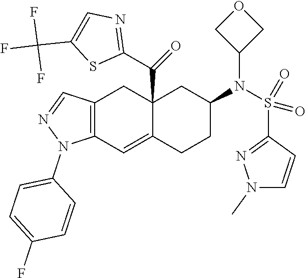 [SMILES: Cn1ccc(S(=O)(=O)N(C2COC2)[C@H]2CCC3=Cc4c(cnn4-c4ccc(F)cc4)C[C@]3(C(=O)c3ncc(C(F)(F)F)s3)C2)n1] | 663 |
| 197 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-pyrazole-3- carboxamide | 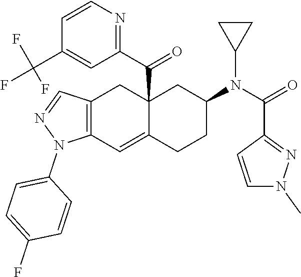 [SMILES: Cn1ccc(C(=O)N(C2CC2)[C@H]2CCC3=Cc4c(cnn4-c4ccc(F)cc4)C[C@]3(C(=O)c3cc(C(F)(F)F)ccn3)C2)n1] | 605.3 |
| 198 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-N-(oxetan-3-yl)-1H- pyrazole-3-sulfonamide | 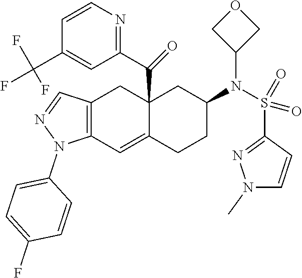 [SMILES: Cn1ccc(S(=O)(=O)N(C2COC2)[C@H]2CCC3=Cc4c(cnn4-c4ccc(F)cc4)C[C@]3(C(=O)c3cc(C(F)(F)F)ccn3)C2)n1] | 657.2 |
| 199 | N-ethyl-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 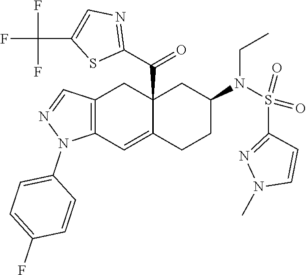 [SMILES: CCN([C@H]1CCC2=Cc3c(cnn3-c3ccc(F)cc3)C[C@]2(C(=O)c2ncc(C(F)(F)F)s2)C1)S(=O)(=O)c1ccn(C)n1] | 635.2 |
| 200 | N-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-2- methyl-2H-1,2,3-triazole-4- sulfonamide | 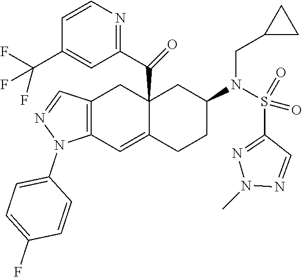 [SMILES: Cn1ncc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1] | 656.2 |
| 201 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-imidazole-4- sulfonamide | 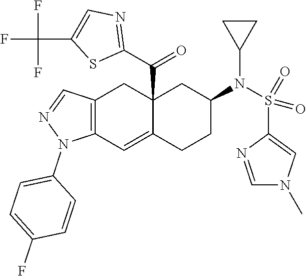 [SMILES: Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)c1] | 647.2 |
| 202 | ((4aS,6S)-6-fluoro-1-(4- fluorophenyl)-6-((1-methyl- 1H-pyrazol-4-yl)sulfonyl)- 1,4,5,6,7,8-hexahydro-4aH- benzo[f]indazol-4a-yl)(4- (trifluoromethyl)pyridin-2- yl)methanone | 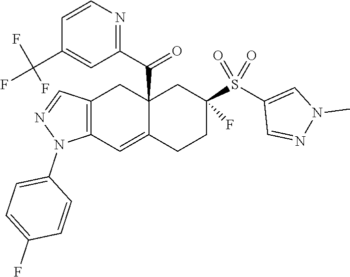 [SMILES: Cn1cc(S(=O)(=O)[C@@]2(F)CCC3=Cc4c(cnn4-c4ccc(F)cc4)C[C@]3(C(=O)c3cc(C(F)(F)F)ccn3)C2)cn1] | 604.2 |
| 203 | N-cyclobutyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-pyrazole-3- sulfonamide | 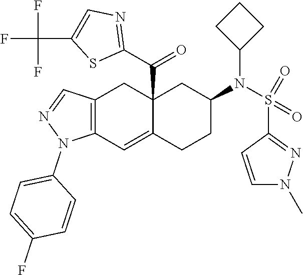 [SMILES: Cn1ccc(S(=O)(=O)N(C2CCC2)[C@H]2CCC3=Cc4c(cnn4-c4ccc(F)cc4)C[C@]3(C(=O)c3ncc(C(F)(F)F)s3)C2)n1] | 661.3 |
| 204 | 4-fluoro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- (oxetan-3- yl)benzenesulfonamide | 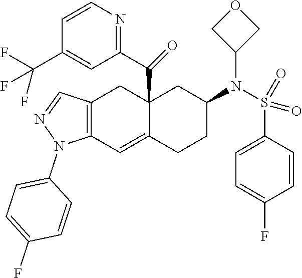 [SMILES: O=C(c1cc(C(F)(F)F)ccn1)[C@]12Cc3cnn(-c4ccc(F)cc4)c3C=C1CC[C@H](N(C1COC1)S(=O)(=O)c1ccc(F)cc1)C2] | 671.2 |
| 205 | 4-fluoro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- (oxetan-3- yl)benzenesulfonamide | 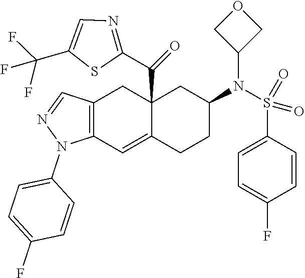 [SMILES: O=C(c1ncc(C(F)(F)F)s1)[C@]12Cc3cnn(-c4ccc(F)cc4)c3C=C1CC[C@H](N(C1COC1)S(=O)(=O)c1ccc(F)cc1)C2] | 677.2 |
| 206 | N-cyclobutyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 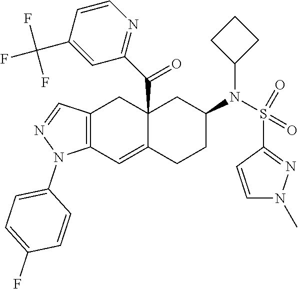 [SMILES: Cn1ccc(S(=O)(=O)N(C2CCC2)[C@H]2CCC3=Cc4c(cnn4-c4ccc(F)cc4)C[C@]3(C(=O)c3cc(C(F)(F)F)ccn3)C2)n1] | 655.3 |
| 207 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(oxetan-3-yl)-1H- pyrazole-4-sulfonamide | 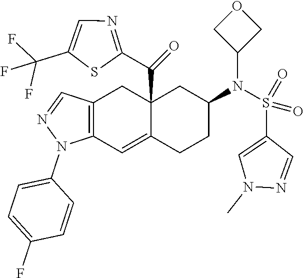 [SMILES: Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)cn1] | 663.2 |
| 208 | 4-chloro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropylbenzenesulfonamide | 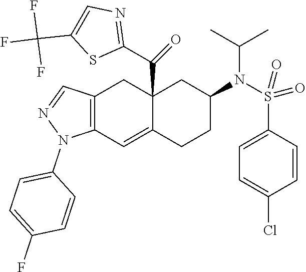 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)cc1] | 679.1 |
| 209 | 4-fluoro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropylbenzenesulfonamide | 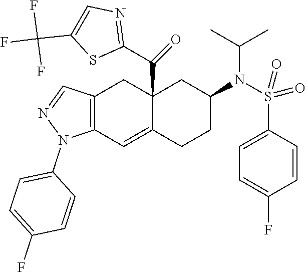 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccc(F)cc1] | 663.2 |
| 210 | 4-fluoro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropylbenzenesulfonamide | 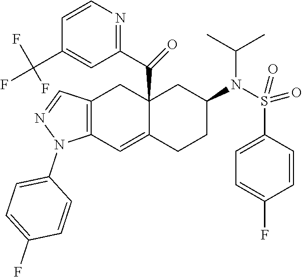 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ccc(F)cc1] | 657.2 |
| 211 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H- pyrazole-4-carboxamide | 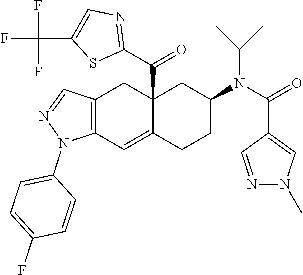 [SMILES: CC(C)N(C(=O)c1cnn(C)c1)[C@H]1CCC2=Cc3c(cnn3-c3ccc(F)cc3)C[C@]2(C(=O)c2ncc(C(F)(F)F)s2)C1] | 613.2 |
| 212 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isobutyl-1-methyl-1H- pyrazole-3-sulfonamide | 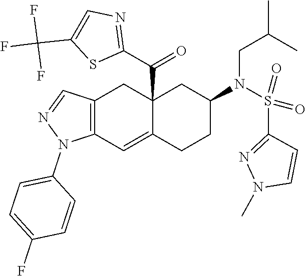 [SMILES: CC(C)CN([C@H]1CCC2=Cc3c(cnn3-c3ccc(F)cc3)C[C@]2(C(=O)c2ncc(C(F)(F)F)s2)C1)S(=O)(=O)c1ccn(C)n1] | 663.2 |
| 213 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-N-(oxetan-3-yl)-1H- pyrazole-4-sulfonamide | 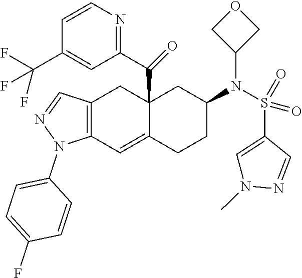 [SMILES: Cn1cc(S(=O)(=O)N(C2COC2)[C@H]2CCC3=Cc4c(cnn4-c4ccc(F)cc4)C[C@]3(C(=O)c3cc(C(F)(F)F)ccn3)C2)cn1] | 657.2 |
| 214 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-1H-imidazole-4- sulfonamide | 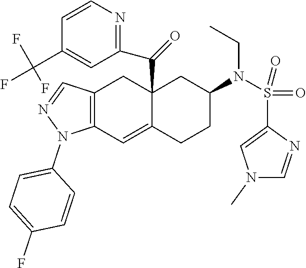 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cn(C)cn1] | 629.2 |
| 215 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isobutyl-1-methyl-1H- pyrazole-3-sulfonamide | 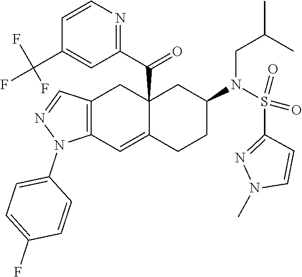 [SMILES: CC(C)CN([C@H]1CCC2=Cc3c(cnn3-c3ccc(F)cc3)C[C@]2(C(=O)c2cc(C(F)(F)F)ccn2)C1)S(=O)(=O)c1ccn(C)n1] | 657.3 |
| 216 | N-ethyl-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-imidazole-4- sulfonamide | 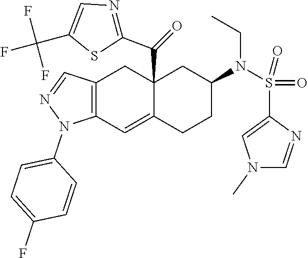 [SMILES: CCN([C@H]1CCC2=Cc3c(cnn3-c3ccc(F)cc3)C[C@]2(C(=O)c2ncc(C(F)(F)F)s2)C1)S(=O)(=O)c1cn(C)cn1] | 635.2 |
| 217 | N-(cyclopropylmethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-imidazole-4- sulfonamide | 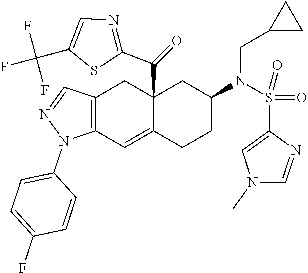 [SMILES: Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)c1] | 661.2 |
| 218 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(oxetan-3-yl)-1H- 1,2,3-triazole-4-sulfonamide | 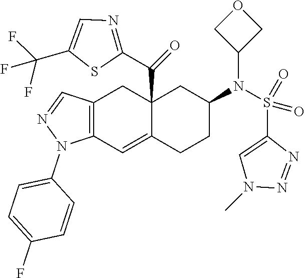 [SMILES: Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)nn1] | 664.1 |
| 219 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-1,2,3-triazole-4- sulfonamide | 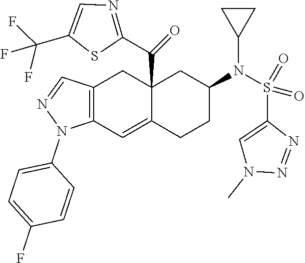 [SMILES: Cn1cc(S(=O)(=O)N(C2CC2)[C@H]2CCC3=Cc4c(cnn4-c4ccc(F)cc4)C[C@]3(C(=O)c3ncc(C(F)(F)F)s3)C2)nn1] | 648.2 |
| 220 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-N-(oxetan-3-yl)-1H- 1,2,3-triazole-4-sulfonamide | 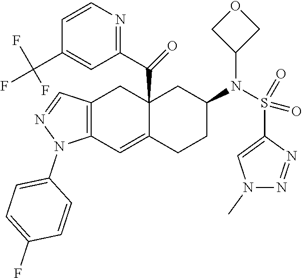 [SMILES: Cn1cc(S(=O)(=O)N(C2COC2)[C@H]2CCC3=Cc4c(cnn4-c4ccc(F)cc4)C[C@]3(C(=O)c3cc(C(F)(F)F)ccn3)C2)nn1] | 658.1 |
| 221 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-1-methyl-1H- imidazole-4-sulfonamide | 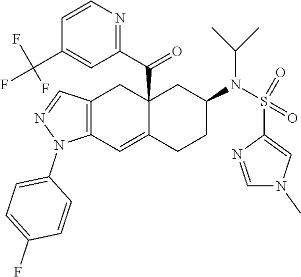 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)cn1] | 643.3 |
| 222 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 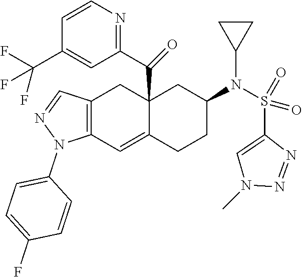 [SMILES: Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)nn1] | 642.2 |
| 223 | 6-chloro-N-cyclopropyl-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)pyridine- 3-sulfonamide | 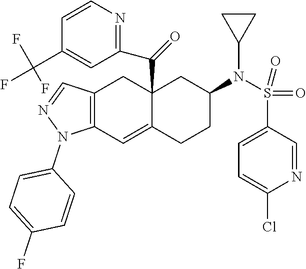 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(Cl)nc1)C2] | 672.1 |
| 224 | N-cyclopropyl-N-((4aS,6S)-1- (4-fluorophenyl)-4a-(4- fluoropicolinoyl)-4,4a,5,6,7,8- hexahydro-1H- benzo[f]indazol-6-yl)-1- methyl-1H-pyrazole-3- sulfonamide | 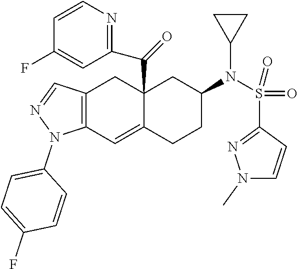 [SMILES: Cn1ccc(S(=O)(=O)N(C2CC2)[C@H]2CCC3=Cc4c(cnn4-c4ccc(F)cc4)C[C@]3(C(=O)c3cc(F)ccn3)C2)n1] | 591.1 |
| 225 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-1-methyl-1H- pyrazole-3-sulfonamide | 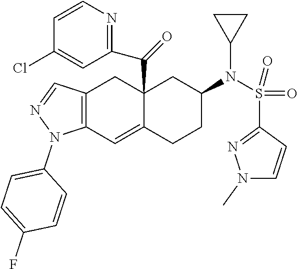 [SMILES: Cn1ccc(S(=O)(=O)N(C2CC2)[C@H]2CCC3=Cc4c(cnn4-c4ccc(F)cc4)C[C@]3(C(=O)c3cc(Cl)ccn3)C2)n1] | 607.2 |
| 226 | N-cyclobutyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-imidazole-4- sulfonamide | 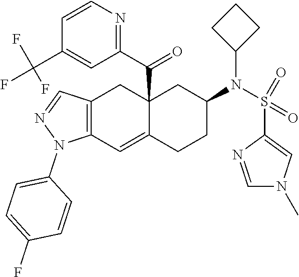 [SMILES: Cn1cnc(S(=O)(=O)N(C2CCC2)[C@H]2CCC3=Cc4c(cnn4-c4ccc(F)cc4)C[C@]3(C(=O)c3cc(C(F)(F)F)ccn3)C2)c1] | 655.3 |
| 227 | N-(3,3-difluorocyclobutyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-pyrazole-3- sulfonamide | 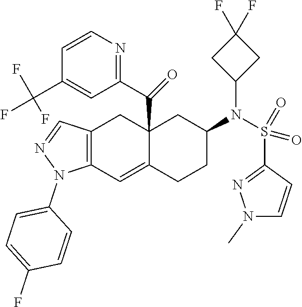 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1] | 691.1 |
| 228 | N-cyclopropyl-4-fluoro-N- ((4aS,6S)-1-(4-fluorophenyl)- 4a-(4-fluoropicolinoyl)- 4,4a,5,6,7,8-hexahydro-1H- benzo[f]indazol-6- yl)benzenesulfonamide | 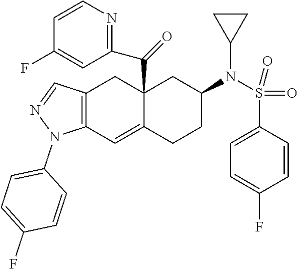 [SMILES: O=C(c1cc(F)ccn1)[C@]12Cc3cnn(-c4ccc(F)cc4)c3C=C1CC[C@H](N(C1CC1)S(=O)(=O)c1ccc(F)cc1)C2] | 605.2 |
| 229 | N-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 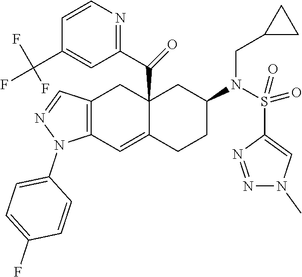 [SMILES: Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)nn1] | 656.3 |
| 230 | N-cyclobutyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 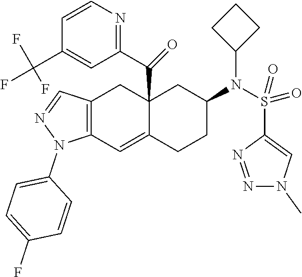 [SMILES: Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)nn1] | 656.3 |
| 231 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 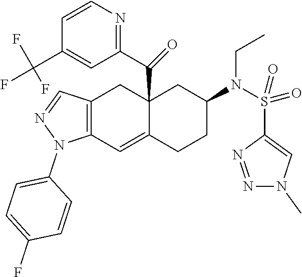 [SMILES: CCN([C@H]1CCC2=Cc3c(cnn3-c3ccc(F)cc3)C[C@]2(C(=O)c2cc(C(F)(F)F)ccn2)C1)S(=O)(=O)c1cn(C)nn1] | 630.2 |
| 232 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-4- fluorobenzenesulfonamide | 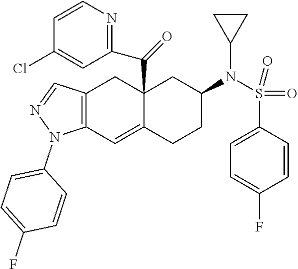 [SMILES: O=C(c1cc(Cl)ccn1)[C@]12Cc3cnn(-c4ccc(F)cc4)c3C=C1CC[C@H](N(C1CC1)S(=O)(=O)c1ccc(F)cc1)C2] | 621.1 |
| 233 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-2-methyl-2H-1,2,3- triazole-4-sulfonamide | 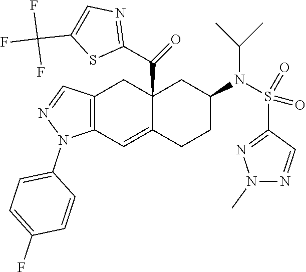 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)n1] | 650.2 |
| 234 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-2-methyl-2H-1,2,3- triazole-4-sulfonamide | 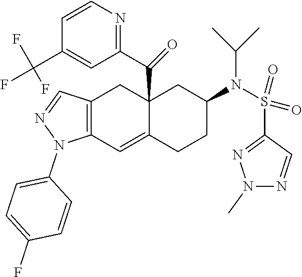 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)n1] | 644.2 |
| 235 | N-cyclobutyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-pyrazole-4- sulfonamide | 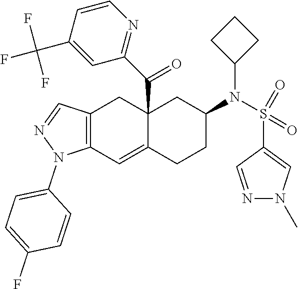 [SMILES: Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)cn1] | 655.3 |
| 236 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- hydroxy-2-methylpropyl)-2- methyl-2H-1,2,3-triazole-4- sulfonamide | 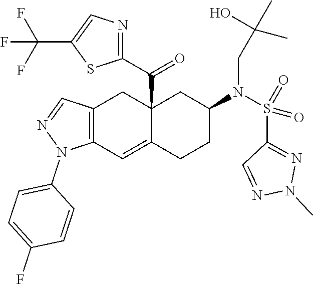 [SMILES: Cn1ncc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1] | 680.2 |
| 237 | N-cyclopentyl-4-fluoro-N- ((4aS,6S)-1-(4-fluorophenyl)- 4a-(4- (trifluoromethyl)picolinoyl)- 4,4a,5,6,7,8-hexahydro-1H- benzo[f]indazol-6- yl)benzenesulfonamide | 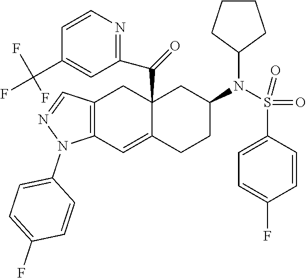 [SMILES: O=C(c1cc(C(F)(F)F)ccn1)[C@]12Cc3cnn(-c4ccc(F)cc4)c3C=C1CC[C@H](N(C1CCCC1)S(=O)(=O)c1ccc(F)cc1)C2] | 683.2 |
| 238 | N-cyclopentyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 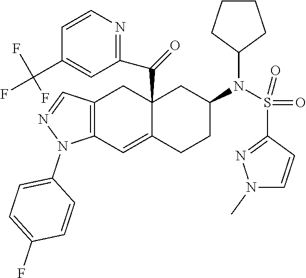 [SMILES: Cn1ccc(S(=O)(=O)N(C2CCCC2)[C@H]2CCC3=Cc4c(cnn4-c4ccc(F)cc4)C[C@]3(C(=O)c3cc(C(F)(F)F)ccn3)C2)n1] | 669.1 |
| 239 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-2- methyl-N-(oxetan-3-yl)-2H- 1,2,3-triazole-4-sulfonamide | 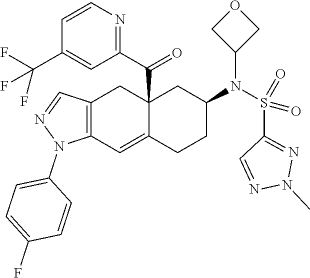 [SMILES: Cn1ncc(S(=O)(=O)N(C2COC2)[C@H]2CCC3=Cc4c(cnn4-c4ccc(F)cc4)C[C@]3(C(=O)c3cc(C(F)(F)F)ccn3)C2)n1] | 658.3 |
| 240 | N-((4aS,6S)-4a-(5- chloropicolinoyl)-1-(4- fluorophenyl)-4,4a,5,6,7,8- hexahydro-1H- benzo[f]indazol-6-yl)-N- cyclopropyl-4- fluorobenzenesulfonamide | 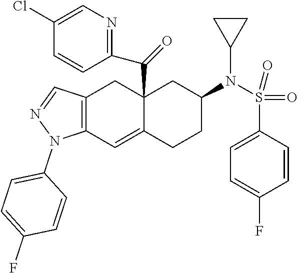 [SMILES: O=C(c1ccc(Cl)cn1)[C@]12Cc3cnn(-c4ccc(F)cc4)c3C=C1CC[C@H](N(C1CC1)S(=O)(=O)c1ccc(F)cc1)C2] | 621.1 |
| 241 | N-((4aS,6S)-1-(4- fluorophenyl)-4a-(4- fluoropicolinoyl)-4,4a,5,6,7,8- hexahydro-1H- benzo[f]indazol-6-yl)-N- isopropyl-1-methyl-1H- pyrazole-3-sulfonamide | 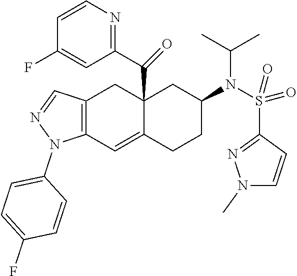 [SMILES: CC(C)N([C@H]1CCC2=Cc3c(cnn3-c3ccc(F)cc3)C[C@]2(C(=O)c2cc(F)ccn2)C1)S(=O)(=O)c1ccn(C)n1] | 593.3 |
| 242 | N-((4aS,6S)-4a-(4- chloropicolinoyl)-1-(4- fluorophenyl)-4,4a,5,6,7,8- hexahydro-1H- benzo[f]indazol-6-yl)-N- isopropyl-1-methyl-1H- pyrazole-3-sulfonamide | 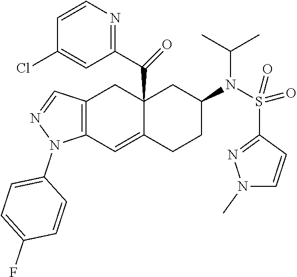 [SMILES: CC(C)N([C@H]1CCC2=Cc3c(cnn3-c3ccc(F)cc3)C[C@]2(C(=O)c2cc(Cl)ccn2)C1)S(=O)(=O)c1ccn(C)n1] | 609.2 |
| 243 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6- yl)cyclopentanesulfonamide | 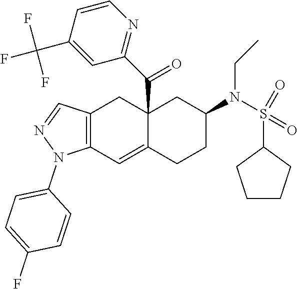 [SMILES: CCN([C@H]1CCC2=Cc3c(cnn3-c3ccc(F)cc3)C[C@]2(C(=O)c2cc(C(F)(F)F)ccn2)C1)S(=O)(=O)C1CCCC1] | 617.3 |
| 244 | N-(cyclopropylmethyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-pyrazole-3- sulfonamide | 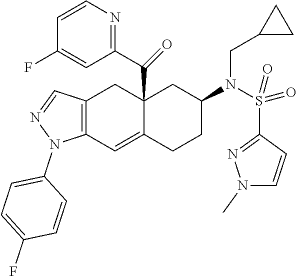 [SMILES: Cn1ccc(S(=O)(=O)N(CC2CC2)[C@H]2CCC3=Cc4c(cnn4-c4ccc(F)cc4)C[C@]3(C(=O)c3cc(F)ccn3)C2)n1] | 605.3 |
| 245 | N-((4aS,6S)-4a-(4- chloropicolinoyl)-1-(4- fluorophenyl)-4,4a,5,6,7,8- hexahydro-1H- benzo[f]indazol-6-yl)-N- (cyclopropylmethyl)-1-methyl- 1H-pyrazole-3-sulfonamide | 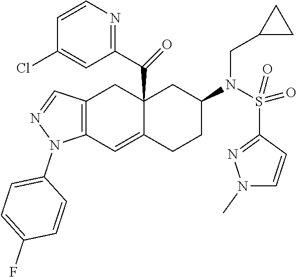 [SMILES: Cn1ccc(S(=O)(=O)N(CC2CC2)[C@H]2CCC3=Cc4c(cnn4-c4ccc(F)cc4)C[C@]3(C(=O)c3cc(Cl)ccn3)C2)n1] | 621.2 |
| 246 | N-((4aS,6S)-4a-(4- chloropicolinoyl)-1-(4- fluorophenyl)-4,4a,5,6,7,8- hexahydro-1H- benzo[f]indazol-6-yl)-N-(2- hydroxy-2-methylpropyl)-1- methyl-1H-pyrazole-4- sulfonamide | 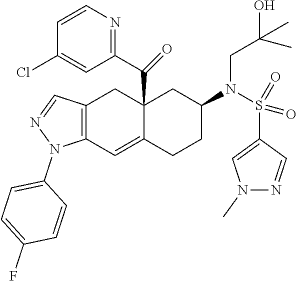 [SMILES: Cn1cc(S(=O)(=O)N(CC(C)(C)O)[C@H]2CCC3=Cc4c(cnn4-c4ccc(F)cc4)C[C@]3(C(=O)c3cc(Cl)ccn3)C2)cn1] | 639.2 |
| 247 | N-cyclopropyl-N-((4aS,6S)- 4a-(4- (difluoromethyl)picolinoyl)-1- (4-fluorophenyl)-4,4a,5,6,7,8- hexahydro-1H- benzo[f]indazol-6-yl)-4- fluorobenzenesulfonamide | 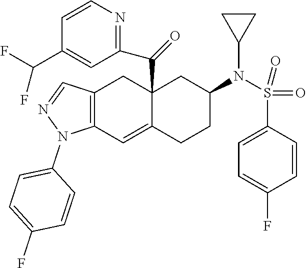 [SMILES: O=C(c1cc(C(F)F)ccn1)[C@]12Cc3cnn(-c4ccc(F)cc4)c3C=C1CC[C@H](N(C1CC1)S(=O)(=O)c1ccc(F)cc1)C2] | 637.2 |
| 248 | N-((4aS,6S)-1-(4- fluorophenyl)-4a-(4- fluoropicolinoyl)-4,4a,5,6,7,8- hexahydro-1H- benzo[f]indazol-6-yl)-N-(2- hydroxy-2-methylpropyl)-1- methyl-1H-pyrazole-4- sulfonamide | 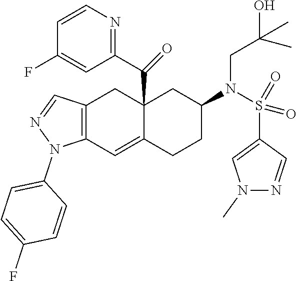 [SMILES: Cn1cc(S(=O)(=O)N(CC(C)(C)O)[C@H]2CCC3=Cc4c(cnn4-c4ccc(F)cc4)C[C@]3(C(=O)c3cc(F)ccn3)C2)cn1] | 623.2 |
| 249 | N-((4aS,6S)-4a-(4- chloropicolinoyl)-1-(4- fluorophenyl)-4,4a,5,6,7,8- hexahydro-1H- benzo[f]indazol-6-yl)-N- isobutyl-1-methyl-1H- pyrazole-3-sulfonamide | 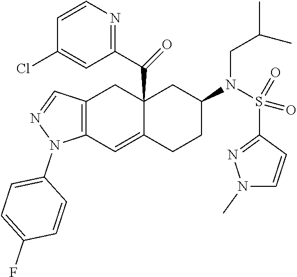 [SMILES: CC(C)CN([C@H]1CCC2=Cc3c(cnn3-c3ccc(F)cc3)C[C@]2(C(=O)c2cc(Cl)ccn2)C1)S(=O)(=O)c1ccn(C)n1] | 623.2 |
| 250 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(2,2,2- trifluoroethyl)-1H-pyrazole-4- sulfonamide | 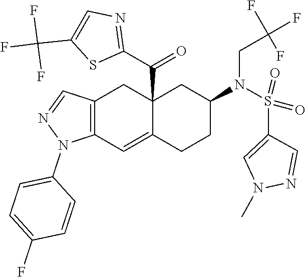 [SMILES: Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)cn1] | 689.2 |
| 251 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-((1- hydroxycyclopropyl)methyl)- 1-methyl-1H-pyrazole-3- sulfonamide | 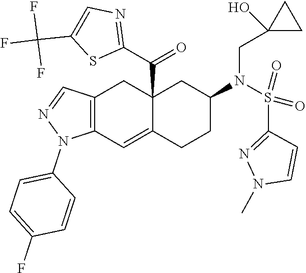 [SMILES: Cn1ccc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1] | 677.2 |
| 252 | N-((4aS,6S)-1-(4- fluorophenyl)-4a-(4- fluoropicolinoyl)-4,4a,5,6,7,8- hexahydro-1H- benzo[f]indazol-6-yl)-N- isobutyl-1-methyl-1H- pyrazole-3-sulfonamide | 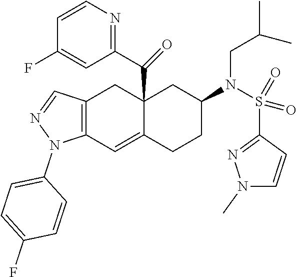 [SMILES: CC(C)CN([C@H]1CCC2=Cc3c(cnn3-c3ccc(F)cc3)C[C@]2(C(=O)c2cc(F)ccn2)C1)S(=O)(=O)c1ccn(C)n1] | 607.3 |
| 253 | N-cyclobutyl-1- (difluoromethyl)-N-((4aS,6S)- 1-(4-fluorophenyl)-4a-(4- (trifluoromethyl)picolinoyl)- 4,4a,5,6,7,8-hexahydro-1H- benzo[f]indazol-6-yl)-1H- pyrazole-3-sulfonamide | 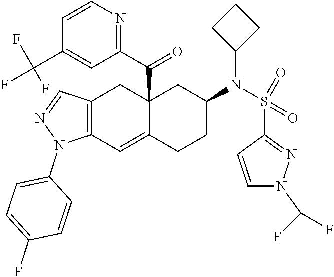 [SMILES: O=C(c1cc(C(F)(F)F)ccn1)[C@]12Cc3cnn(-c4ccc(F)cc4)c3C=C1CC[C@H](N(C1CCC1)S(=O)(=O)c1ccn(C(F)F)n1)C2] | 691.3 |
| 254 | 4-fluoro-N-((4aS,6S)-1-(4- fluorophenyl)-4a-(4- fluoropicolinoyl)-4,4a,5,6,7,8- hexahydro-1H- benzo[f]indazol-6-yl)-N-((1- hydroxycyclopropyl)methyl) benzenesulfonamide | 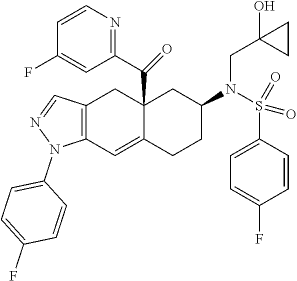 [SMILES: O=C(c1cc(F)ccn1)[C@]12Cc3cnn(-c4ccc(F)cc4)c3C=C1CC[C@H](N(CC1(O)CC1)S(=O)(=O)c1ccc(F)cc1)C2] | 635.2 |
| 255 | N-((4aS,6S)-1-(4- fluorophenyl)-4a-(4- fluoropicolinoyl)-4,4a,5,6,7,8- hexahydro-1H- benzo[f]indazol-6-yl)-N-((1- hydroxycyclopropyl)methyl)- 1-methyl-1H-pyrazole-3- sulfonamide | 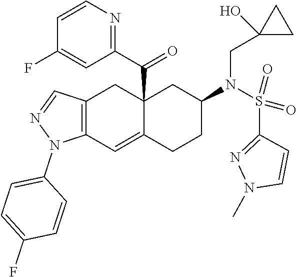 [SMILES: Cn1ccc(S(=O)(=O)N(CC2(O)CC2)[C@H]2CCC3=Cc4c(cnn4-c4ccc(F)cc4)C[C@]3(C(=O)c3cc(F)ccn3)C2)n1] | 621.3 |
| 256 | N-cyclobutyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-2- methyl-2H-1,2,3-triazole-4- sulfonamide | 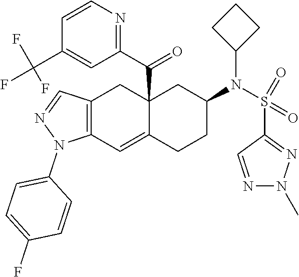 [SMILES: Cn1ncc(S(=O)(=O)N(C2CCC2)[C@H]2CCC3=Cc4c(cnn4-c4ccc(F)cc4)C[C@]3(C(=O)c3cc(C(F)(F)F)ccn3)C2)n1] | 656.3 |
| 257 | N-((4aS,6S)-4a-(4- chloropicolinoyl)-1-(4- fluorophenyl)-4,4a,5,6,7,8- hexahydro-1H- benzo[f]indazol-6-yl)-N-((1- hydroxycyclopropyl)methyl)- 1-methyl-1H-pyrazole-3- sulfonamide | 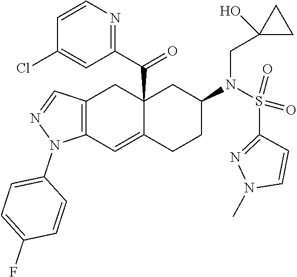 [SMILES: Cn1ccc(S(=O)(=O)N(CC2(O)CC2)[C@H]2CCC3=Cc4c(cnn4-c4ccc(F)cc4)C[C@]3(C(=O)c3cc(Cl)ccn3)C2)n1] | 637.2 |
| 258 | N-cyclopropyl-N-((4aS,6S)- 4a-(4- (difluoromethyl)picolinoyl)-1- (4-fluorophenyl)-4,4a,5,6,7,8- hexahydro-1H- benzo[f]indazol-6-yl)-1- methyl-1H-pyrazole-3- sulfonamide | 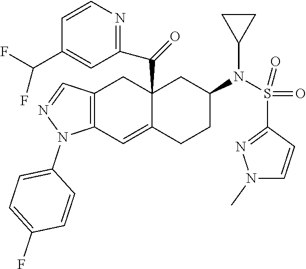 [SMILES: Cn1ccc(S(=O)(=O)N(C2CC2)[C@H]2CCC3=Cc4c(cnn4-c4ccc(F)cc4)C[C@]3(C(=O)c3cc(C(F)F)ccn3)C2)n1] | 623.2 |
| 259 | N-((4aS,6S)-4a-(5- chloropyrimidine-2-carbonyl)- 1-(4-fluorophenyl)- 4,4a,5,6,7,8-hexahydro-1H- benzo[f]indazol-6-yl)-N- cyclopropyl-1-methyl-1H- pyrazole-3-sulfonamide | 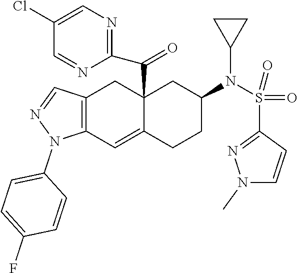 [SMILES: Cn1ccc(S(=O)(=O)N(C2CC2)[C@H]2CCC3=Cc4c(cnn4-c4ccc(F)cc4)C[C@]3(C(=O)c3ncc(Cl)cn3)C2)n1] | 608.2 |
| 260 | N-((4aS,6S)-1-(4- fluorophenyl)-4a-(4- fluoropicolinoyl)-4,4a,5,6,7,8- hexahydro-1H- benzo[f]indazol-6-yl)-1- methyl-N-(2,2,2- trifluoroethyl)-1H-pyrazole-4- sulfonamide | 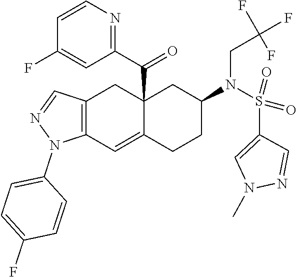 [SMILES: Cn1cc(S(=O)(=O)N(CC(F)(F)F)[C@H]2CCC3=Cc4c(cnn4-c4ccc(F)cc4)C[C@]3(C(=O)c3cc(F)ccn3)C2)cn1] | 633.2 |
| 261 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-imidazole-2- sulfonamide | 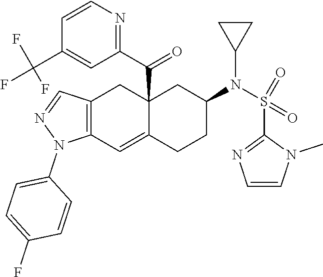 [SMILES: Cn1ccnc1S(=O)(=O)N(C1CC1)[C@H]1CCC2=Cc3c(cnn3-c3ccc(F)cc3)C[C@]2(C(=O)c2cc(C(F)(F)F)ccn2)C1] | 641.2 |
| 262 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-2-methyl-2H- 1,2,3-triazole-4-sulfonamide | 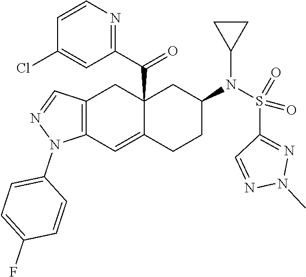 [SMILES: Cn1ncc(S(=O)(=O)N(C2CC2)[C@H]2CCC3=Cc4c(cnn4-c4ccc(F)cc4)C[C@]3(C(=O)c3cc(Cl)ccn3)C2)n1] | 609.2 |
| 263 | N-cyclopropyl-N-((4aS,6S)-1- (4-fluorophenyl)-4a-(4- fluoropicolinoyl)-4,4a,5,6,7,8- hexahydro-1H- benzo[f]indazol-6-yl)-2- methyl-2H-1,2,3-triazole-4- sulfonamide | 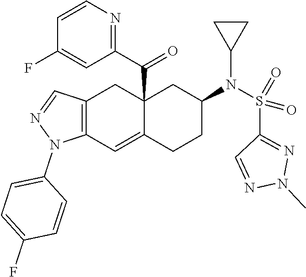 [SMILES: Cn1ncc(S(=O)(=O)N(C2CC2)[C@H]2CCC3=Cc4c(cnn4-c4ccc(F)cc4)C[C@]3(C(=O)c3cc(F)ccn3)C2)n1] | 592.3 |
| 264 | ((4aS,6S)-1-(4-fluorophenyl)- 6-((1-methyl-1H-pyrazol-3- yl)sulfonyl)-1,4,5,6,7,8- hexahydro-4aH- benzo[f]indazol-4a-yl)(5- (trifluoromethyl)thiazol-2- yl)methanone | 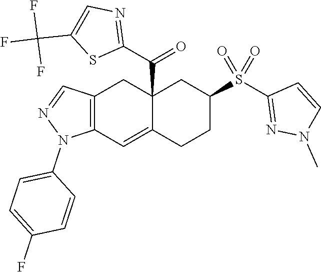 [SMILES: Cn1ccc(S(=O)(=O)[C@H]2CCC3=Cc4c(cnn4-c4ccc(F)cc4)C[C@]3(C(=O)c3ncc(C(F)(F)F)s3)C2)n1] | 592 |
| 265 | N-cyclopropyl-N-((4aS,6S)-1- (4-fluorophenyl)-4a-(4- fluoropicolinoyl)-4,4a,5,6,7,8- hexahydro-1H- benzo[f]indazol-6-yl)-1- methyl-1H-1,2,3-triazole-4- sulfonamide | 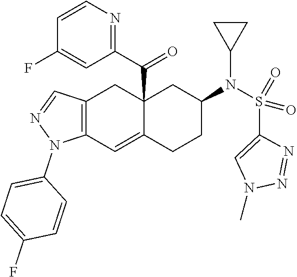 [SMILES: Cn1cc(S(=O)(=O)N(C2CC2)[C@H]2CCC3=Cc4c(cnn4-c4ccc(F)cc4)C[C@]3(C(=O)c3cc(F)ccn3)C2)nn1] | 592.3 |
| 266 | N-cyclopropyl-N-((4aS,6S)-1- (4-fluorophenyl)-4a-(5 (trifluoromethyl)thiazole-2- carbonyl)-4,4a,5,6,7,8- hexahydro-1H- benzo[f]indazol-6-yl)-1- methyl-1H-imidazole-2- sulfonamide | 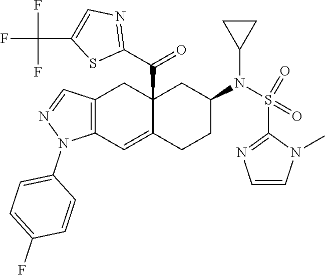 [SMILES: Cn1ccnc1S(=O)(=O)N(C1CC1)[C@H]1CCC2=Cc3c(cnn3-c3ccc(F)cc3)C[C@]2(C(=O)c2ncc(C(F)(F)F)s2)C1] | 647.2 |
| 267 | N-(3,3-difluorocyclobutyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-pyrazole-3- sulfonamide | 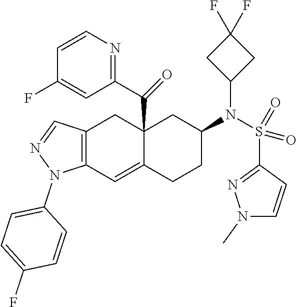 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(F)ccn3)C2)n1] | 641.2 |
| 268 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-1-methyl-1H- 1,2,3-triazole-4-sulfonamide | 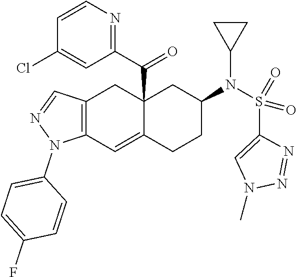 [SMILES: Cn1cc(S(=O)(=O)N(C2CC2)[C@H]2CCC3=Cc4c(cnn4-c4ccc(F)cc4)C[C@]3(C(=O)c3cc(Cl)ccn3)C2)nn1] | 609.2 |
| 269 | N-((4aS,6S)-4a-(4- chloropicolinoyl)-1-(4- fluorophenyl)-4,4a,5,6,7,8- hexahydro-1H- benzo[f]indazol-6-yl)-N- isopropyl-2-methyl-2H-1,2,3- triazole-4-sulfonamide | 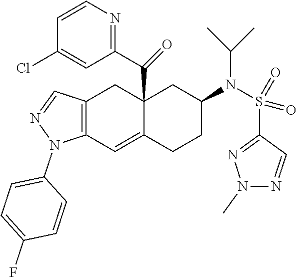 [SMILES: CC(C)N([C@H]1CCC2=Cc3c(cnn3-c3ccc(F)cc3)C[C@]2(C(=O)c2cc(Cl)ccn2)C1)S(=O)(=O)c1cnn(C)n1] | 610.1 |
| 270 | N-(3,3-difluorocyclobutyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 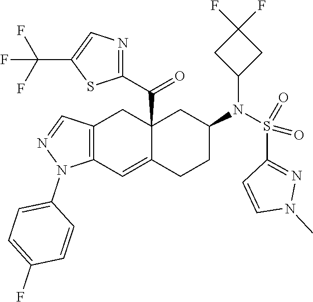 [SMILES: Cn1ccc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1] | 697.1 |
| 271 | 6-chloro-N-cyclopropyl-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)pyridine- 3-sulfonamide | 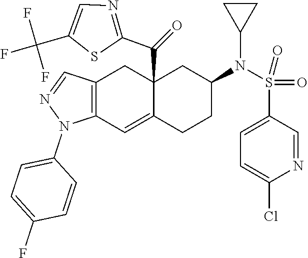 [SMILES: O=C(c1ncc(C(F)(F)F)s1)[C@]12Cc3cnn(-c4ccc(F)cc4)c3C=C1CC[C@H](N(C1CC1)S(=O)(=O)c1ccc(Cl)nc1)C2] | 678 |
| 272 | ((4aS,6S)-1-(4-fluorophenyl)- 6-((1-methyl-1H-pyrazol-3- yl)sulfonyl)-1,4,5,6,7,8- hexahydro-4aH- benzo[f]indazol-4a-yl)(5- (trifluoromethyl)pyridin-2- yl)methanone | 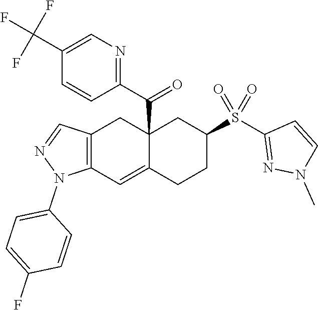 [SMILES: Cn1ccc(S(=O)(=O)[C@H]2CCC3=Cc4c(cnn4-c4ccc(F)cc4)C[C@]3(C(=O)c3ccc(C(F)(F)F)cn3)C2)n1] | 586.2 |
| 273 | N-(cyclopropylmethyl)-4- fluoro-N-((4aS,6S)-1-(4- fluorophenyl)-4a-(4- (trifluoromethyl)picolinoyl)- 4,4a,5,6,7,8-hexahydro-1H- benzo[f]indazol-6- yl)benzenesulfonamide | 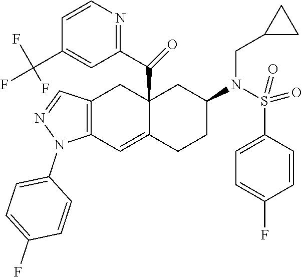 [SMILES: O=C(c1cc(C(F)(F)F)ccn1)[C@]12Cc3cnn(-c4ccc(F)cc4)c3C=C1CC[C@H](N(CC1CC1)S(=O)(=O)c1ccc(F)cc1)C2] | 669.2 |
| 274 | N-(cyclopropylmethyl)-4- fluoro-N-((4aS,6S)-1-(4- fluorophenyl)-4a-(4- fluoropicolinoyl)-4,4a,5,6,7,8- hexahydro-1H- benzo[f]indazol-6- yl)benzenesulfonamide | 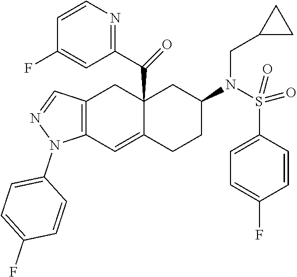 [SMILES: O=C(c1cc(F)ccn1)[C@]12Cc3cnn(-c4ccc(F)cc4)c3C=C1CC[C@H](N(CC1CC1)S(=O)(=O)c1ccc(F)cc1)C2] | 619.3 |
| 275 | N-((4aS,6S)-4a-(4- chloropicolinoyl)-1-(4- fluorophenyl)-4,4a,5,6,7,8- hexahydro-1H- benzo[f]indazol-6-yl)-N- (cyclopropylmethyl)-4- fluorobenzenesulfonamide | 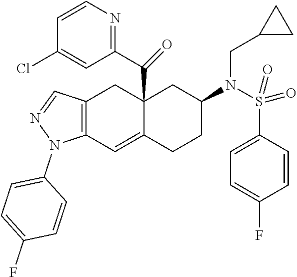 [SMILES: O=C(c1cc(Cl)ccn1)[C@]12Cc3cnn(-c4ccc(F)cc4)c3C=C1CC[C@H](N(CC1CC1)S(=O)(=O)c1ccc(F)cc1)C2] | 635.2 |
| 276 | N-((4aS,6S)-4a-(4- chloropicolinoyl)-1-(4- fluorophenyl)-4,4a,5,6,7,8- hexahydro-1H- benzo[f]indazol-6-yl)-4-fluoro- N-((1- hydroxycyclopropyl)methyl) benzenesulfonamide | 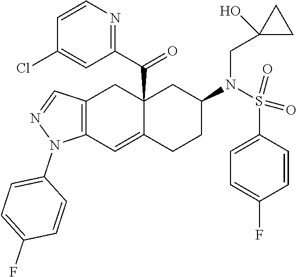 [SMILES: O=C(c1cc(Cl)ccn1)[C@]12Cc3cnn(-c4ccc(F)cc4)c3C=C1CC[C@H](N(CC1(O)CC1)S(=O)(=O)c1ccc(F)cc1)C2] | 651.1 |
| 277 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- ((1s,3R)-3-hydroxy-3- methylcyclobutyl)-1-methyl- 1H-pyrazole-3-sulfonamide | 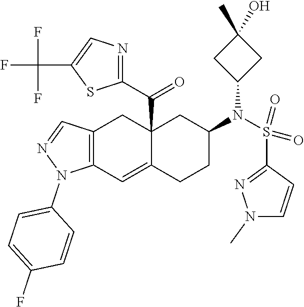 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@](C)(O)C2)n1] | 691.1 |
| 278 | N-((4aS,6S)-1-(4- fluorophenyl)-4a-(4- fluoropicolinoyl)-4,4a,5,6,7,8- hexahydro-1H- benzo[f]indazol-6-yl)-N- isopropyl-2-methyl-2H-1,2,3- triazole-4-sulfonamide | 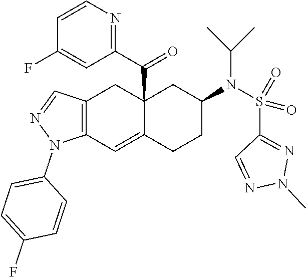 [SMILES: CC(C)N([C@H]1CCC2=Cc3c(cnn3-c3ccc(F)cc3)C[C@]2(C(=O)c2cc(F)ccn2)C1)S(=O)(=O)c1cnn(C)n1] | 594.2 |
| 279 | 6-chloro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropylpyridine-3- sulfonamide | 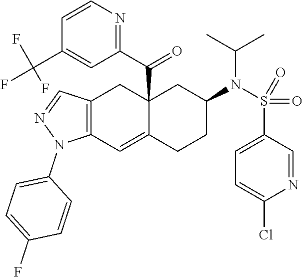 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1] | 674.2 |
| 280 | N-((4aS,6S)-4a-(4- chloropicolinoyl)-1-(4- fluorophenyl)-4,4a,5,6,7,8- hexahydro-1H- benzo[f]indazol-6-yl)-1- methyl-N-(2,2,2- trifluoroethyl)-1H-pyrazole-4- sulfonamide | 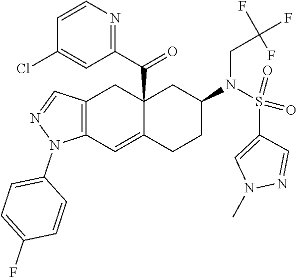 [SMILES: Cn1cc(S(=O)(=O)N(CC(F)(F)F)[C@H]2CCC3=Cc4c(cnn4-c4ccc(F)cc4)C[C@]3(C(=O)c3cc(Cl)ccn3)C2)cn1] | 649.1 |
| 281 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropylcyclopentanecarbox- amide | 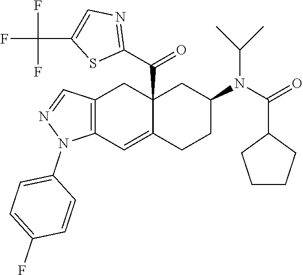 [SMILES: CC(C)N(C(=O)C1CCCC1)[C@H]1CCC2=Cc3c(cnn3-c3ccc(F)cc3)C[C@]2(C(=O)c2ncc(C(F)(F)F)s2)C1] | 601.3 |
| 282 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H-1,2,3- triazole-4-sulfonamide | 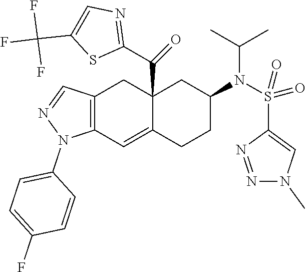 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cn(C)nn1] | 650.2 |
| 283 | 6-chloro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropylpyridine-3- sulfonamide | 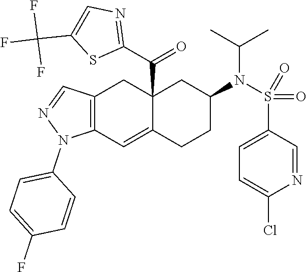 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)nc1] | 680 |
| 284 | N-((4aS,6S)-1-(4- fluorophenyl)-4a-(4- fluoropicolinoyl)-4,4a,5,6,7,8- hexahydro-1H- benzo[f]indazol-6-yl)-N- ((1s,3R)-3-hydroxy-3- methylcyclobutyl)-1-methyl- 1H-pyrazole-3-sulfonamide | 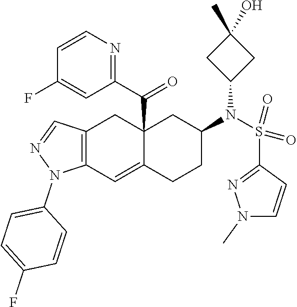 [SMILES: Cn1ccc(S(=O)(=O)N([C@H]2CCC3=Cc4c(cnn4-c4ccc(F)cc4)C[C@]3(C(=O)c3cc(F)ccn3)C2)[C@H]2C[C@@](C)(O)C2)n1] | 635.2 |
| 285 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-6- (trifluoromethyl)pyridine-3- sulfonamide | 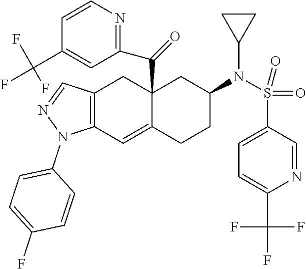 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(C(F)(F)F)nc1)C2] | 706.2 |
| 286 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-1-methyl-1H-1,2,3- triazole-4-sulfonamide | 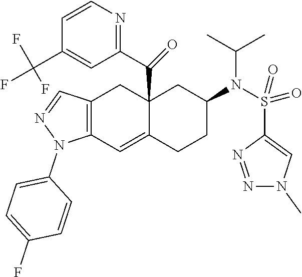 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)nn1] | 644.2 |
| 287 | 5-chloro-N-cyclopropyl-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)pyridine- 2-sulfonamide | 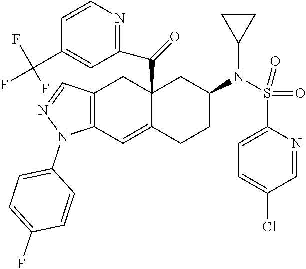 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(Cl)cn1)C2] | 672.1 |
| 288 | N-((4aS,6S)-4a-(4- chloropicolinoyl)-1-(4- fluorophenyl)-4,4a,5,6,7,8- hexahydro-1H- benzo[f]indazol-6-yl)-N- ((1s,3R)-3-hydroxy-3- methylcyclobutyl)-1-methyl- 1H-pyrazole-3-sulfonamide | 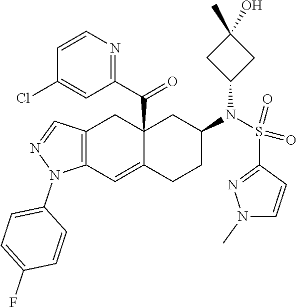 [SMILES: Cn1ccc(S(=O)(=O)N([C@H]2CCC3=Cc4c(cnn4-c4ccc(F)cc4)C[C@]3(C(=O)c3cc(Cl)ccn3)C2)[C@H]2C[C@@](C)(O)C2)n1] | 651.3 |
| 289 | ((4aS,6S)-6-fluoro-1-(4- fluorophenyl)-6-((1-methyl- 1H-pyrazol-3-yl)sulfonyl)- 1,4,5,6,7,8-hexahydro-4aH- benzo[f]indazol-4a-yl)(5- (trifluoromethyl)pyridin-2- yl)methanone | 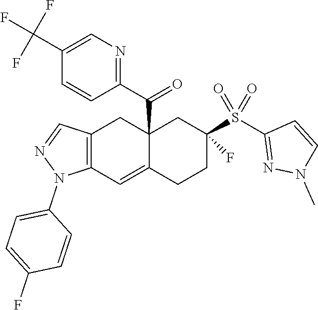 [SMILES: Cn1ccc(S(=O)(=O)[C@@]2(F)CCC3=Cc4c(cnn4-c4ccc(F)cc4)C[C@]3(C(=O)c3ccc(C(F)(F)F)cn3)C2)n1] | 604.2 |
| 290 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(2,2,2- trifluoroethyl)-1H-1,2,3- triazole-4-sulfonamide | 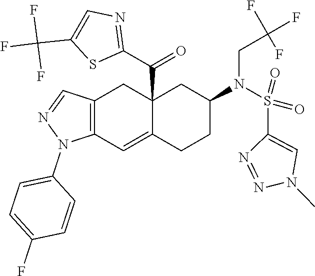 [SMILES: Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)nn1] | 690 |
| 291 | N-cyclopropyl-4-fluoro-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6- yl)benzamide | 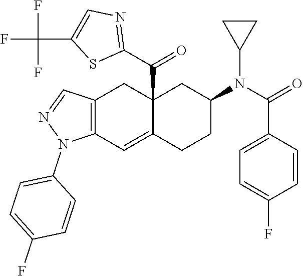 [SMILES: O=C(c1ccc(F)cc1)N(C1CC1)[C@H]1CCC2=Cc3c(cnn3-c3ccc(F)cc3)C[C@]2(C(=O)c2ncc(C(F)(F)F)s2)C1] | 625.3 |
| 292 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-((1- hydroxycyclopropyl)methyl)- 2-methyl-2H-1,2,3-triazole-4- sulfonamide | 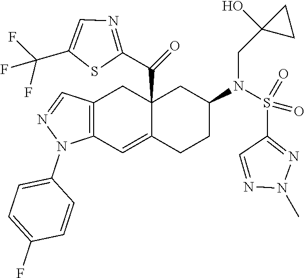 [SMILES: Cn1ncc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1] | 678.1 |
| 293 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(2,2,2- trifluoroethyl)-1H-imidazole- 4-sulfonamide | 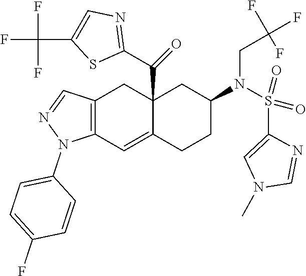 [SMILES: Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)c1] | 689 |
| 294 | 6-chloro-N- (cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)pyridine- 3-sulfonamide | 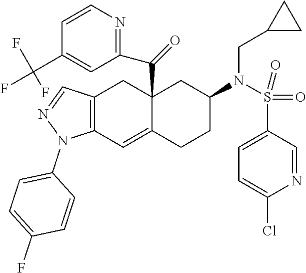 [SMILES: O=C(c1cc(C(F)(F)F)ccn1)[C@]12Cc3cnn(-c4ccc(F)cc4)c3C=C1CC[C@H](N(CC1CC1)S(=O)(=O)c1ccc(Cl)nc1)C2] | 686 |
| 295 | N-((4aS,6S)-1-(4- fluorophenyl)-4a-(4- fluoropicolinoyl)-4,4a,5,6,7,8- hexahydro-1H- benzo[f]indazol-6-yl)-N- isopropyl-1-methyl-1H-1,2,3- triazole-4-sulfonamide | 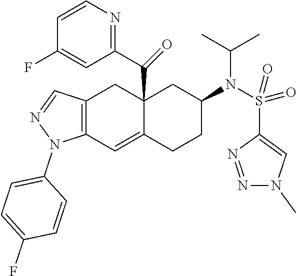 [SMILES: CC(C)N([C@H]1CCC2=Cc3c(cnn3-c3ccc(F)cc3)C[C@]2(C(=O)c2cc(F)ccn2)C1)S(=O)(=O)c1cn(C)nn1] | 594.2 |
| 296 | N-((4aS,6S)-4a-(4- chloropicolinoyl)-1-(4- fluorophenyl)-4,4a,5,6,7,8- hexahydro-1H- benzo[f]indazol-6-yl)-N- isopropyl-1-methyl-1H-1,2,3- triazole-4-sulfonamide | 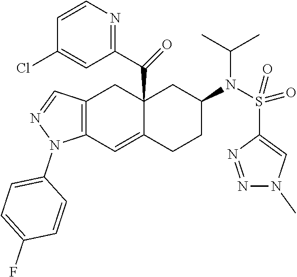 [SMILES: CC(C)N([C@H]1CCC2=Cc3c(cnn3-c3ccc(F)cc3)C[C@]2(C(=O)c2cc(Cl)ccn2)C1)S(=O)(=O)c1cn(C)nn1] | 610.2 |
| 297 | N-(cyclopropylmethyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-1,2,3-triazole-4- sulfonamide | 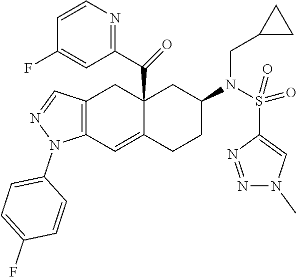 [SMILES: Cn1cc(S(=O)(=O)N(CC2CC2)[C@H]2CCC3=Cc4c(cnn4-c4ccc(F)cc4)C[C@]3(C(=O)c3cc(F)ccn3)C2)nn1] | 606.2 |
| 298 | N-(cyclopropylmethyl)-N- ((4aS,6S)-4a-(4- (difluoromethyl)picolinoyl)-1- (4-fluorophenyl)-4,4a,5,6,7,8- hexahydro-1H- benzo[f]indazol-6-yl)-1- methyl-1H-pyrazole-3- sulfonamide | 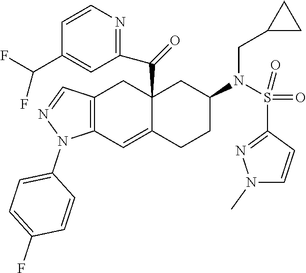 [SMILES: Cn1ccc(S(=O)(=O)N(CC2CC2)[C@H]2CCC3=Cc4c(cnn4-c4ccc(F)cc4)C[C@]3(C(=O)c3cc(C(F)F)ccn3)C2)n1] | 637.2 |
| 299 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H- imidazole-4-sulfonamide | 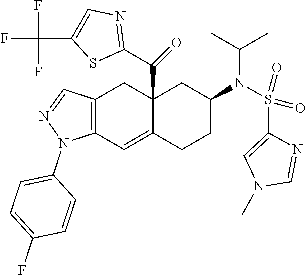 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cn(C)cn1] | 649.2 |
| 300 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-2- methyl-N-(2,2,2- trifluoroethyl)-2H-1,2,3- triazole-4-sulfonamide | 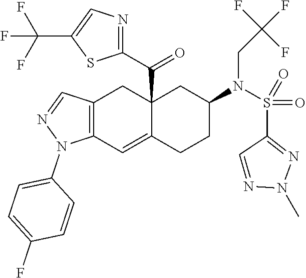 [SMILES: Cn1ncc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1] | 690 |
| 301 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- hydroxy-2-methylpropyl)-1- methyl-1H-1,2,3-triazole-4- sulfonamide | 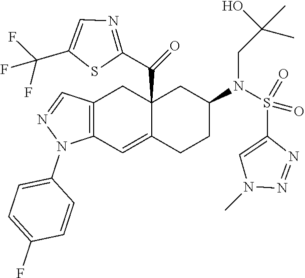 [SMILES: Cn1cc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)nn1] | 680.2 |
| 302 | 6-chloro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N-(2- hydroxy-2- methylpropyl)pyridine-3- sulfonamide | 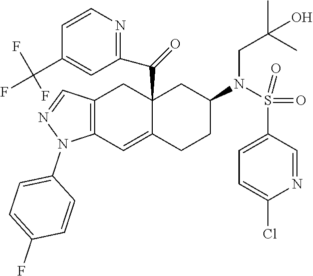 [SMILES: CC(C)(O)CN([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1] | 704.2 |
| 303 | 6-chloro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- hydroxy-2- methylpropyl)pyridine-3- sulfonamide | 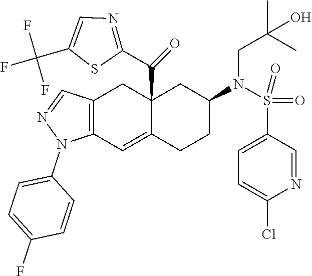 [SMILES: CC(C)(O)CN([C@H]1CCC2=Cc3c(cnn3-c3ccc(F)cc3)C[C@]2(C(=O)c2ncc(C(F)(F)F)s2)C1)S(=O)(=O)c1ccc(Cl)nc1] | 710 |
| 304 | 6-chloro-N-((4aS,6S)-1-(4- fluorophenyl)-4a-(4- fluoropicolinoyl)-4,4a,5,6,7,8- hexahydro-1H- benzo[f]indazol-6-yl)-N-(2- hydroxy-2- methylpropyl)pyridine-3- sulfonamide | 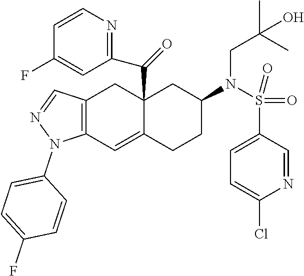 [SMILES: CC(C)(O)CN([C@H]1CCC2=Cc3c(cnn3-c3ccc(F)cc3)C[C@]2(C(=O)c2cc(F)ccn2)C1)S(=O)(=O)c1ccc(Cl)nc1] | 654.1 |
| 305 | N-cyclopropyl-N-((4aS,6S)-1- (4-fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 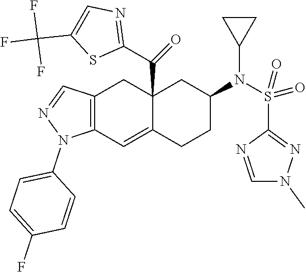 [SMILES: Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)n1] | 648.2 |
| 306 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 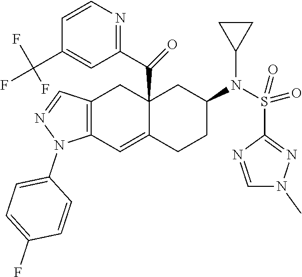 [SMILES: Cn1cnc(S(=O)(=O)N(C2CC2)[C@H]2CCC3=Cc4c(cnn4-c4ccc(F)cc4)C[C@]3(C(=O)c3cc(C(F)(F)F)ccn3)C2)n1] | 642.2 |
| 307 | N-cyclopropyl-N-((4aS,6S)-1- (4-fluorophenyl)-4a-(4- (trifluoromethyl)picolinoyl)- 4,4a,5,6,7,8-hexahydro-1H- benzo[f]indazol-6-yl)-1,2- dimethyl-1H-imidazole-5- sulfonamide | 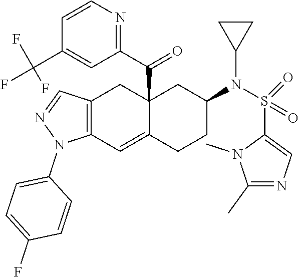 [SMILES: Cc1ncc(S(=O)(=O)N(C2CC2)[C@H]2CCC3=Cc4c(cnn4-c4ccc(F)cc4)C[C@]3(C(=O)c3cc(C(F)(F)F)ccn3)C2)n1C] | 655.3 |
| 308 | ((4aS,6S)-6-((1-ethyl-1H- pyrazol-3-yl)sulfonyl)-1-(4- fluorophenyl)-1,4,5,6,7,8- hexahydro-4aH- benzo[f]indazol-4a-yl)(5- (trifluoromethyl)pyridin-2- yl)methanone | 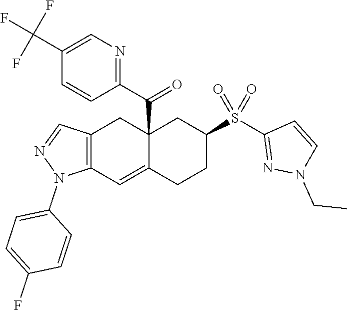 [SMILES: CCn1ccc(S(=O)(=O)[C@H]2CCC3=Cc4c(cnn4-c4ccc(F)cc4)C[C@]3(C(=O)c3ccc(C(F)(F)F)cn3)C2)n1] | 600.3 |
| 309 | ((4aS,6S)-6-((1-cyclopropyl- 1H-pyrazol-3-yl)sulfonyl)-1- (4-fluorophenyl)-1,4,5,6,7,8- hexahydro-4aH- benzo[f]indazol-4a-yl)(5- (trifluoromethyl)pyridin-2- yl)methanone | 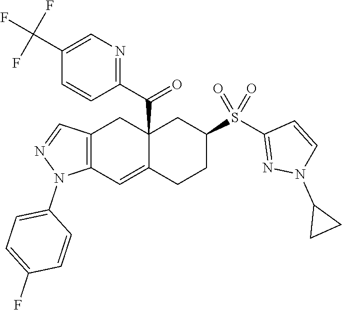 [SMILES: O=C(c1ccc(C(F)(F)F)cn1)[C@]12Cc3cnn(-c4ccc(F)cc4)c3C=C1CC[C@H](S(=O)(=O)c1ccn(C3CC3)n1)C2] | 612.1 |
| 310 | ((4aS,6S)-1-(4-fluorophenyl)- 6-((1-isopropyl-1H-pyrazol-3- yl)sulfonyl)-1,4,5,6,7,8- hexahydro-4aH- benzo[f]indazol-4a-yl)(5- (trifluoromethyl)pyridin-2- yl)methanone | 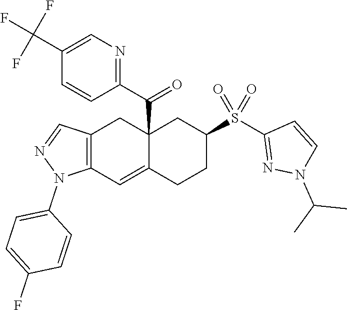 [SMILES: CC(C)n1ccc(S(=O)(=O)[C@H]2CCC3=Cc4c(cnn4-c4ccc(F)cc4)C[C@]3(C(=O)c3ccc(C(F)(F)F)cn3)C2)n1] | 614.2 |
| 311 | N-(2,2-difluoroethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 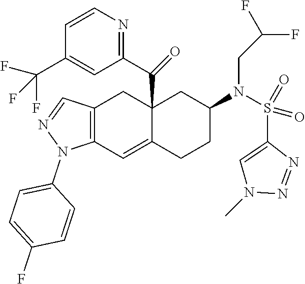 [SMILES: Cn1cc(S(=O)(=O)N(CC(F)F)[C@H]2CCC3=Cc4c(cnn4-c4ccc(F)cc4)C[C@]3(C(=O)c3cc(C(F)(F)F)ccn3)C2)nn1] | 666.2 |
| 312 | 6-chloro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- (2,2,2-trifluoroethyl)pyridine- 3-sulfonamide | 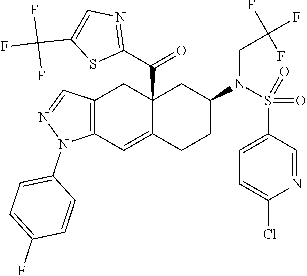 [SMILES: O=C(c1ncc(C(F)(F)F)s1)[C@]12Cc3cnn(-c4ccc(F)cc4)c3C=C1CC[C@H](N(CC(F)(F)F)S(=O)(=O)c1ccc(Cl)nc1)C2] | 720.1 |
| 313 | N-(2,2-difluoroethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-pyrazole-3- sulfonamide | 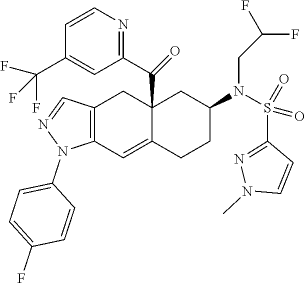 [SMILES: Cn1ccc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1] | 665.2 |
| 314 | ((4aS,6S)-1-(4-fluorophenyl)- 6-((1-methyl-1H-pyrazol-4- yl)sulfonyl)-1,4,5,6,7,8- hexahydro-4aH- benzo[f]indazol-4a-yl)(4- fluoropyridin-2-yl)methanone | 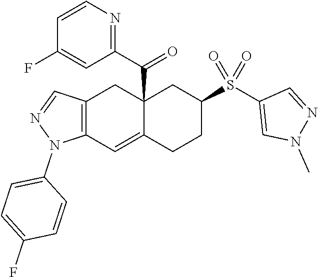 [SMILES: Cn1cc(S(=O)(=O)[C@H]2CCC3=Cc4c(cnn4-c4ccc(F)cc4)C[C@]3(C(=O)c3cc(F)ccn3)C2)cn1] | 536.2 |
| 315 | N-(cyclopropylmethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 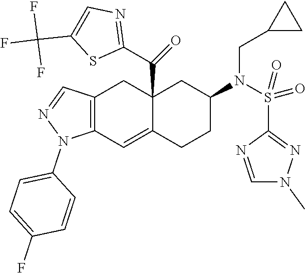 [SMILES: Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)n1] | 662.2 |
| 316 | N-(cyclopropylmethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 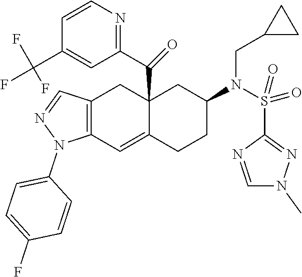 [SMILES: Cn1cnc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1] | 656.2 |
| 317 | (4-chloropyridin-2- yl)((4aS,6S)-1-(4- fluorophenyl)-6-((1-methyl- 1H-pyrazol-4-yl)sulfonyl)- 1,4,5,6,7,8-hexahydro-4aH- benzo[f]indazol-4a- yl)methanone | 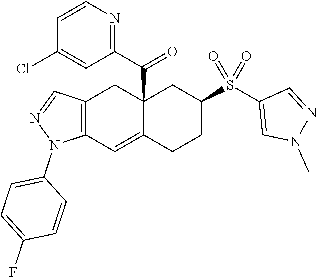 [SMILES: Cn1cc(S(=O)(=O)[C@H]2CCC3=Cc4c(cnn4-c4ccc(F)cc4)C[C@]3(C(=O)c3cc(Cl)ccn3)C2)cn1] | 552.1 |
| 318 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-N-(2,2,2- trifluoroethyl)-1H-1,2,3- triazole-4-sulfonamide | 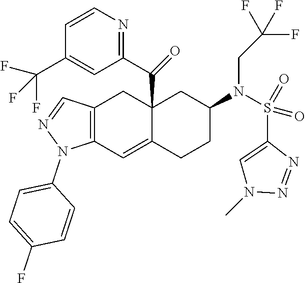 [SMILES: Cn1cc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)nn1] | 684.2 |
| 319 | 6-chloro-N- (cyclopropylmethyl)-N- ((4aS,6S)-1-(4-fluorophenyl)- 4a-(4-fluoropicolinoyl)- 4,4a,5,6,7,8-hexahydro-1H- benzo[f]indazol-6-yl)pyridine- 3-sulfonamide | 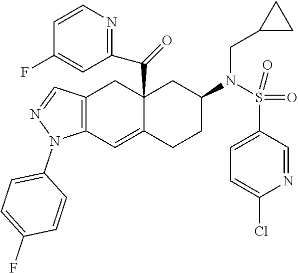 [SMILES: O=C(c1cc(F)ccn1)[C@]12Cc3cnn(-c4ccc(F)cc4)c3C=C1CC[C@H](N(CC1CC1)S(=O)(=O)c1ccc(Cl)nc1)C2] | 636.1 |
| 320 | 6-chloro-N-cyclopropyl-N- ((4aS,6S)-1-(4-fluorophenyl)- 4a-(4-fluoropicolinoyl)- 4,4a,5,6,7,8-hexahydro-1H- benzo[f]indazol-6-yl)pyridine- 3-sulfonamide | 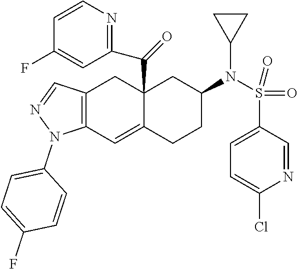 [SMILES: O=C(c1cc(F)ccn1)[C@]12Cc3cnn(-c4ccc(F)cc4)c3C=C1CC[C@H](N(C1CC1)S(=O)(=O)c1ccc(Cl)nc1)C2] | 622.2 |
| 321 | 6-chloro-N-((4aS,6S)-4a-(4- chloropicolinoyl)-1-(4- fluorophenyl)-4,4a,5,6,7,8- hexahydro-1H- benzo[f]indazol-6-yl)-N- cyclopropylpyridine-3- sulfonamide | 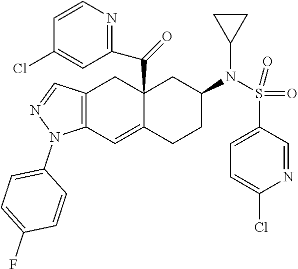 [SMILES: O=C(c1cc(Cl)ccn1)[C@]12Cc3cnn(-c4ccc(F)cc4)c3C=C1CC[C@H](N(C1CC1)S(=O)(=O)c1ccc(Cl)nc1)C2] | 638 |
| 322 | 6-chloro-N-((4aS,6S)-1-(4- fluorophenyl)-4a-(4- fluoropicolinoyl)-4,4a,5,6,7,8- hexahydro-1H- benzo[f]indazol-6-yl)-N- isopropylpyridine-3- sulfonamide | 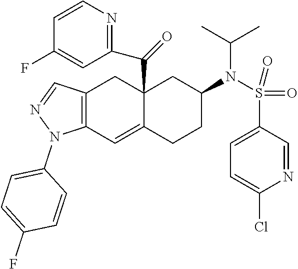 [SMILES: CC(C)N([C@H]1CCC2=Cc3c(cnn3-c3ccc(F)cc3)C[C@]2(C(=O)c2cc(F)ccn2)C1)S(=O)(=O)c1ccc(Cl)nc1] | 624.1 |
| 323 | 6-chloro-N-((4aS,6S)-4a-(4- chloropicolinoyl)-1-(4- fluorophenyl)-4,4a,5,6,7,8- hexahydro-1H- benzo[f]indazol-6-yl)-N- isopropylpyridine-3- sulfonamide | 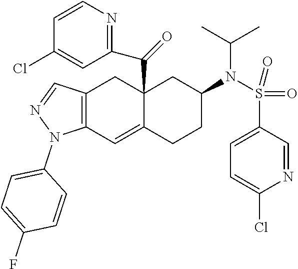 [SMILES: CC(C)N([C@H]1CCC2=Cc3c(cnn3-c3ccc(F)cc3)C[C@]2(C(=O)c2cc(Cl)ccn2)C1)S(=O)(=O)c1ccc(Cl)nc1] | 640.2 |
| 324 | N-(3,3-difluorocyclobutyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 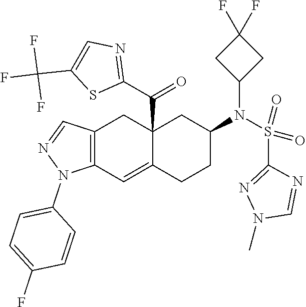 [SMILES: Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1] | 698.2 |
| 325 | N-(3,3-difluorocyclobutyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-imidazole-4- sulfonamide | 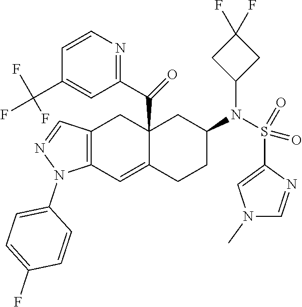 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)c1] | 691.1 |
| 326 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- ((1r,3S)-3-hydroxy-3- methylcyclobutyl)-1-methyl- 1H-pyrazole-3-sulfonamide | 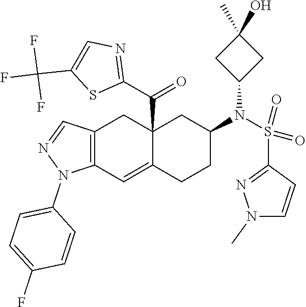 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](C)(O)C2)n1] | 691.2 |
| 327 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- isopropyl-1-methyl-1H-1,2,4- triazole-3-sulfonamide | 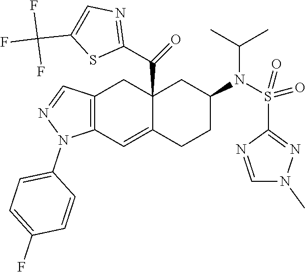 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ncn(C)n1] | 650.2 |
| 328 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- isopropyl-1-methyl-1H-1,2,4- triazole-3-sulfonamide | 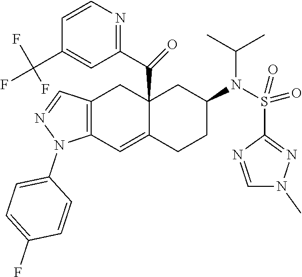 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ncn(C)n1] | 644.2 |
| 329 | N-(3,3-difluorocyclobutyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 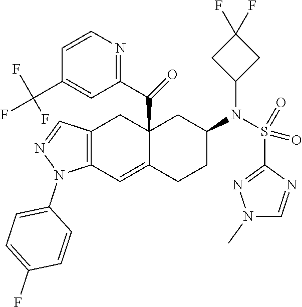 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1] | 692.2 |
| 330 | N-(2,2-difluoroethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 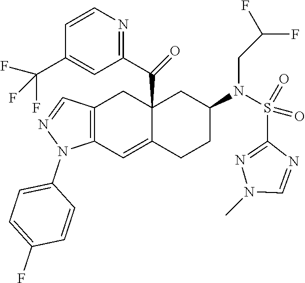 [SMILES: Cn1cnc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1] | 666.2 |
| 331 | N-(2,2-difluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 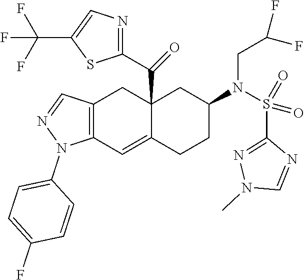 [SMILES: Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)n1] | 672 |
| 332 | N-(3,3-difluorocyclobutyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-imidazole-4- sulfonamide | 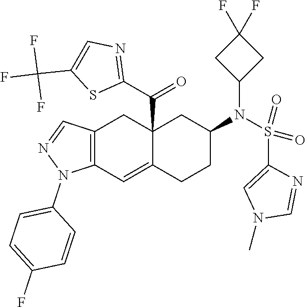 [SMILES: Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)c1] | 697.1 |
| 333 | 5-chloro-N-cyclopropyl-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)pyridine- 2-sulfonamide | 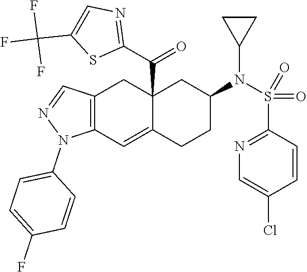 [SMILES: O=C(c1ncc(C(F)(F)F)s1)[C@]12Cc3cnn(-c4ccc(F)cc4)c3C=C1CC[C@H](N(C1CC1)S(=O)(=O)c1ccc(Cl)cn1)C2] | 678.1 |
| 334 | 5-chloro-N-cyclopropyl-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)pyridine- 3-sulfonamide | 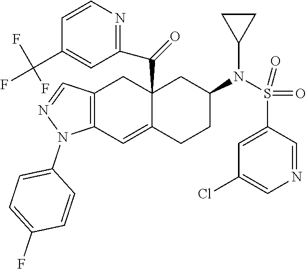 [SMILES: O=C(c1cc(C(F)(F)F)ccn1)[C@]12Cc3cnn(-c4ccc(F)cc4)c3C=C1CC[C@H](N(C1CC1)S(=O)(=O)c1cncc(Cl)c1)C2] | 672.1 |
| 335 | N-(3,3-difluorocyclobutyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-imidazole-4- sulfonamide | 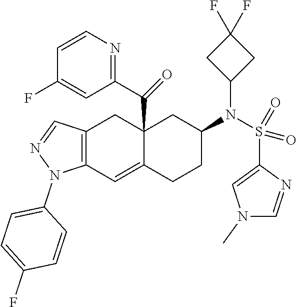 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)c1] | 641.2 |
| 336 | N-cyclopropyl-N-((4aS,6S)-1- (4-fluorophenyl)-4a-(5- (trifluoromethyl)thiazole-2- carbonyl)-4,4a,5,6,7,8- hexahydro-1H- benzo[f]indazol-6-yl)-1,2- dimethyl-1H-imidazole-4- sulfonamide | 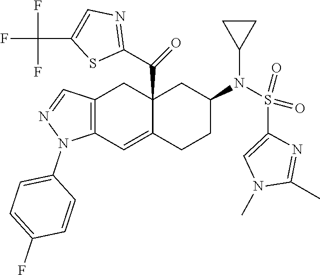 [SMILES: Cc1nc(S(=O)(=O)N(C2CC2)[C@H]2CCC3=Cc4c(cnn4-c4ccc(F)cc4)C[C@]3(C(=O)c3ncc(C(F)(F)F)s3)C2)cn1C] | 661.3 |
| 337 | N-(2,2-difluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 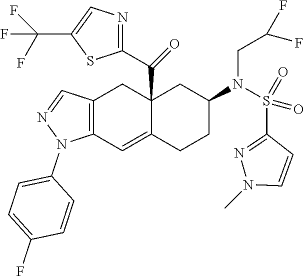 [SMILES: Cn1ccc(S(=O)(=O)N(CC(F)F)[C@H]2CCC3=Cc4c(cnn4-c4ccc(F)cc4)C[C@]3(C(=O)c3ncc(C(F)(F)F)s3)C2)n1] | 671.2 |
| 338 | N-(2,2-difluoroethyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-pyrazole-3- sulfonamide | 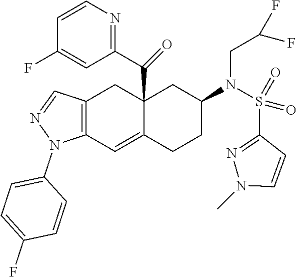 [SMILES: Cn1ccc(S(=O)(=O)N(CC(F)F)[C@H]2CCC3=Cc4c(cnn4-c4ccc(F)cc4)C[C@]3(C(=O)c3cc(F)ccn3)C2)n1] | 615.2 |
| 339 | N-((4aS,6S)-4a-(4- chloropicolinoyl)-1-(4- fluorophenyl)-4,4a,5,6,7,8- hexahydro-1H- benzo[f]indazol-6-yl)-N-(3,3- difluorocyclobutyl)-1-methyl- 1H-imidazole-4-sulfonamide | 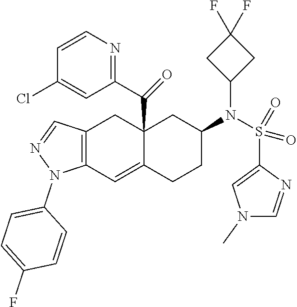 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)c1] | 657.2 |
| 340 | ((4aR)-1-(4-fluorophenyl)-6- (((1-methyl-1H-pyrazol-4- yl)sulfonyl)methyl)- 1,4,5,6,7,8-hexahydro-4aH- benzo[f]indazol-4a-yl)(5- (trifluoromethyl)pyridin-2- yl)methanone | 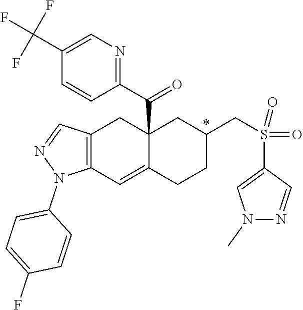 [SMILES: Cn1cc(S(=O)(=O)CC2CCC3=Cc4c(cnn4-c4ccc(F)cc4)C[C@]3(C(=O)c3ccc(C(F)(F)F)cn3)C2)cn1] | 600.1 |
| 341 | 6-chloro-N-(2,2-difluoroethyl)- N-((4aS,6S)-1-(4- fluorophenyl)-4a-(4- (trifluoromethyl)picolinoyl)- 4,4a,5,6,7,8-hexahydro-1H- benzo[f]indazol-6-yl)pyridine- 3-sulfonamide | 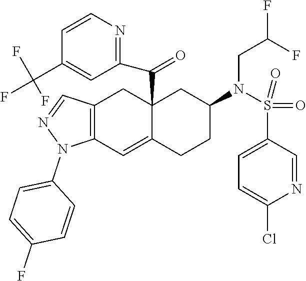 [SMILES: O=C(c1cc(C(F)(F)F)ccn1)[C@]12Cc3cnn(-c4ccc(F)cc4)c3C=C1CC[C@H](N(CC(F)F)S(=O)(=O)c1ccc(Cl)nc1)C2] | 696.1 |
| 342 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- ((1s,3R)-3-methoxy-3- methylcyclobutyl)-1-methyl- 1H-pyrazole-3-sulfonamide | 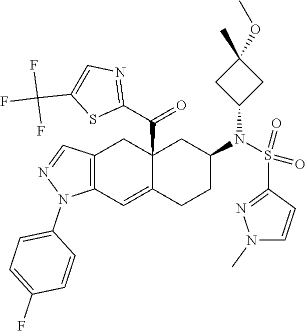 [SMILES: CO[C@]1(C)C[C@H](N([C@H]2CCC3=Cc4c(cnn4-c4ccc(F)cc4)C[C@]3(C(=O)c3ncc(C(F)(F)F)s3)C2)S(=O)(=O)c2ccn(C)n2)C1] | 705.3 |
| 343 | 4-fluoro-N-((4aS,6S)-1-(4- fluorophenyl)-4a-(4- fluoropicolinoyl)-4,4a,5,6,7,8- hexahydro-1H- benzo[f]indazol-6-yl)-N- ((1s,3R)-3-hydroxy-3- methylcyclobutyl)benzenesulfon- amide | 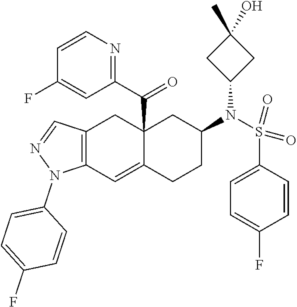 [SMILES: C[C@]1(O)C[C@@H](N([C@H]2CCC3=Cc4c(cnn4-c4ccc(F)cc4)C[C@]3(C(=O)c3cc(F)ccn3)C2)S(=O)(=O)c2ccc(F)cc2)C1] | 649.2 |
| 344 | N-((4aS,6S)-4a-(4- chloropicolinoyl)-1-(4- fluorophenyl)-4,4a,5,6,7,8- hexahydro-1H- benzo[f]indazol-6-yl)-4-fluoro- N-((1s,3R)-3-hydroxy-3- methylcyclobutyl)benzenesulfon- amide | 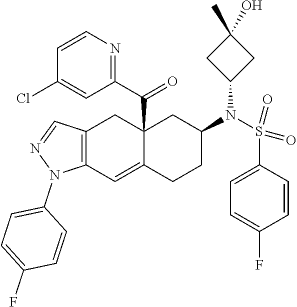 [SMILES: C[C@]1(O)C[C@@H](N([C@H]2CCC3=Cc4c(cnn4-c4ccc(F)cc4)C[C@]3(C(=O)c3cc(Cl)ccn3)C2)S(=O)(=O)c2ccc(F)cc2)C1] | 665.2 |
| 345 | N-((4aS,6S)-4a-(4- chloropicolinoyl)-1-(4- fluorophenyl)-4,4a,5,6,7,8- hexahydro-1H- benzo[f]indazol-6-yl)-N-(2,2- difluoroethyl)-1-methyl-1H- pyrazole-3-sulfonamide | 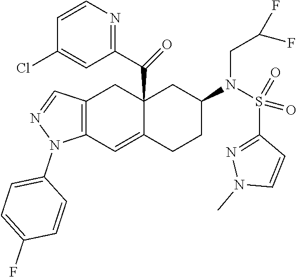 [SMILES: Cn1ccc(S(=O)(=O)N(CC(F)F)[C@H]2CCC3=Cc4c(cnn4-c4ccc(F)cc4)C[C@]3(C(=O)c3cc(Cl)ccn3)C2)n1] | 631.1 |
| 346 | 6-chloro-N-(3,3- difluorocyclobutyl)-N- ((4aS,6S)-1-(4-fluorophenyl)- 4a-(4-fluoropicolinoyl)- 4,4a,5,6,7,8-hexahydro-1H- benzo[f]indazol-6-yl)pyridine- 3-sulfonamide | 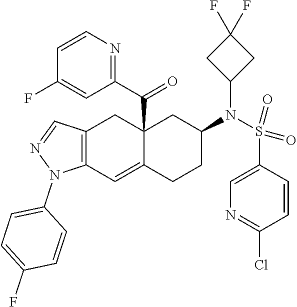 [SMILES: O=C(c1cc(F)ccn1)[C@]12Cc3cnn(-c4ccc(F)cc4)c3C=C1CC[C@H](N(C1CC(F)(F)C1)S(=O)(=O)c1ccc(Cl)nc1)C2] | 672.1 |
| 347 | N-cyclopropyl-N-((4aS,6S)-1- (4-fluorophenyl)-4a-(4- fluoropicolinoyl)-4,4a,5,6,7,8- hexahydro-1H- benzo[f]indazol-6-yl)-1- methyl-1H-1,2,4-triazole-3- sulfonamide | 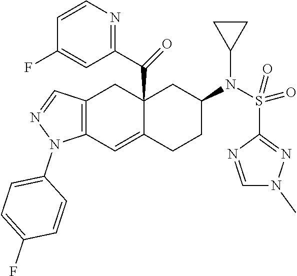 [SMILES: Cn1cnc(S(=O)(=O)N(C2CC2)[C@H]2CCC3=Cc4c(cnn4-c4ccc(F)cc4)C[C@]3(C(=O)c3cc(F)ccn3)C2)n1] | 592.2 |
| 348 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-1-methyl-1H- 1,2,4-triazole-3-sulfonamide | 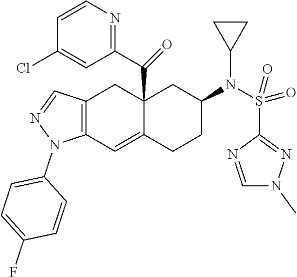 [SMILES: Cn1cnc(S(=O)(=O)N(C2CC2)[C@H]2CCC3=Cc4c(cnn4-c4ccc(F)cc4)C[C@]3(C(=O)c3cc(Cl)ccn3)C2)n1] | 608.2 |
| 349 | N-((4aS,6S)-4a-(4- chloropicolinoyl)-1-(4- fluorophenyl)-4,4a,5,6,7,8- hexahydro-1H- benzo[f]indazol-6-yl)-N-(3,3- difluorocyclobutyl)-1-methyl- 1H-pyrazole-3-sulfonamide | 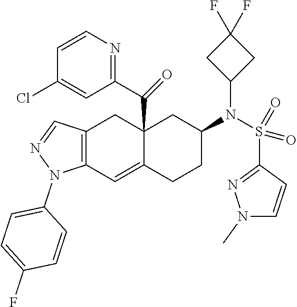 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1] | 657.2 |
| 350 | N-(2,2-difluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-1,2,3-triazole-4- sulfonamide | 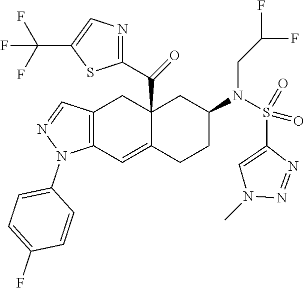 [SMILES: Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)nn1] | 672 |
| 351 | 6-chloro-N-(2,2-difluoroethyl)- N-((4aS,6S)-1-(4- fluorophenyl)-4a-(4- fluoropicolinoyl)-4,4a,5,6,7,8- hexahydro-1H- benzo[f]indazol-6-yl)pyridine- 3-sulfonamide | 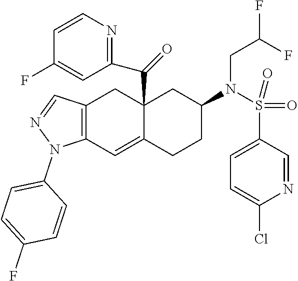 [SMILES: O=C(c1cc(F)ccn1)[C@]12Cc3cnn(-c4ccc(F)cc4)c3C=C1CC[C@H](N(CC(F)F)S(=O)(=O)c1ccc(Cl)nc1)C2] | 646.2 |
| 352 | 6-chloro-N-((4aS,6S)-4a-(4- chloropicolinoyl)-1-(4- fluorophenyl)-4,4a,5,6,7,8- hexahydro-1H- benzo[f]indazol-6-yl)-N-(2,2- difluoroethyl)pyridine-3- sulfonamide | 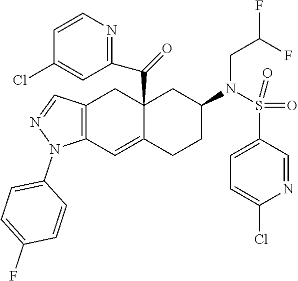 [SMILES: O=C(c1cc(Cl)ccn1)[C@]12Cc3cnn(-c4ccc(F)cc4)c3C=C1CC[C@H](N(CC(F)F)S(=O)(=O)c1ccc(Cl)nc1)C2] | 662 |
| 353 | ((4aR)-1-(4-fluorophenyl)-6- (((1-methyl-1H-pyrazol-3- yl)sulfonyl)methyl)- 1,4,5,6,7,8-hexahydro-4aH- benzo[f]indazol-4a-yl)(4- (trifluoromethyl)pyridin-2- yl)methanone | 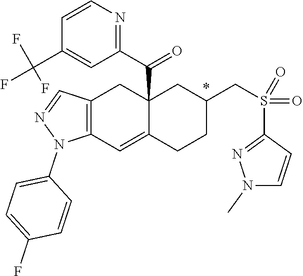 [SMILES: Cn1ccc(S(=O)(=O)CC2CCC3=Cc4c(cnn4-c4ccc(F)cc4)C[C@]3(C(=O)c3cc(C(F)(F)F)ccn3)C2)n1] | 600.1 |
| 354 | N-(3,3-difluorocyclobutyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-1,2,4-triazole-3- sulfonamide | 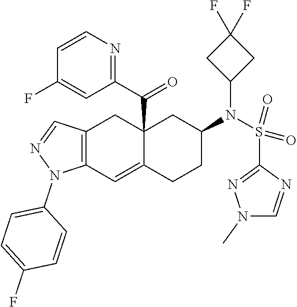 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)n1] | 642.1 |
| 355 | N-((4aS,6S)-4a-(4- chloropicolinoyl)-1-(4- fluorophenyl)-4,4a,5,6,7,8- hexahydro-1H- benzo[f]indazol-6-yl)-N-(3,3- difluorocyclobutyl)-1-methyl- 1H-1,2,4-triazole-3- sulfonamide | 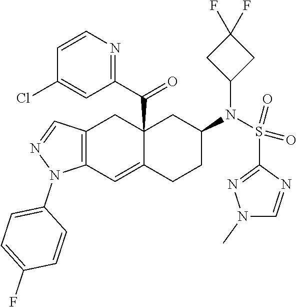 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1] | 658.2 |
| 356 | N-ethyl-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 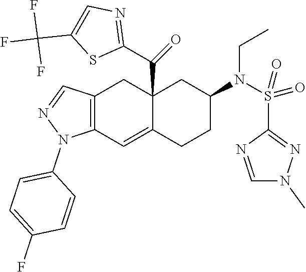 [SMILES: CCN([C@H]1CCC2=Cc3c(cnn3-c3ccc(F)cc3)C[C@]2(C(=O)c2ncc(C(F)(F)F)s2)C1)S(=O)(=O)c1ncn(C)n1] | 636.2 |
| 357 | N-ethyl-N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 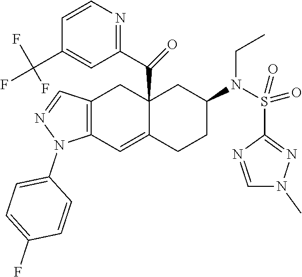 [SMILES: CCN([C@H]1CCC2=Cc3c(cnn3-c3ccc(F)cc3)C[C@]2(C(=O)c2cc(C(F)(F)F)ccn2)C1)S(=O)(=O)c1ncn(C)n1] | 630.2 |
| 358 | N-ethyl-N-((4aS,6S)-1-(4- fluorophenyl)-4a-(4- fluoropicolinoyl)-4,4a,5,6,7,8- hexahydro-1H- benzo[f]indazol-6-yl)-1- methyl-1H-1,2,4-triazole-3- sulfonamide | 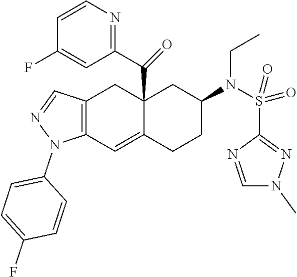 [SMILES: CCN([C@H]1CCC2=Cc3c(cnn3-c3ccc(F)cc3)C[C@]2(C(=O)c2cc(F)ccn2)C1)S(=O)(=O)c1ncn(C)n1] | 580.3 |
| 359 | N-((4aS,6S)-4a-(4- chloropicolinoyl)-1-(4- fluorophenyl)-4,4a,5,6,7,8- hexahydro-1H- benzo[f]indazol-6-yl)-N-ethyl- 1-methyl-1H-1,2,4-triazole-3- sulfonamide | 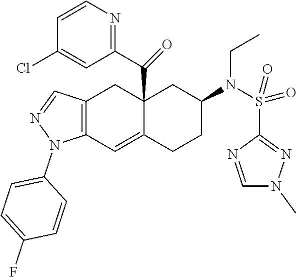 [SMILES: CCN([C@H]1CCC2=Cc3c(cnn3-c3ccc(F)cc3)C[C@]2(C(=O)c2cc(Cl)ccn2)C1)S(=O)(=O)c1ncn(C)n1] | 596.2 |
| 360 | ((4aR)-1-(4-fluorophenyl)-6- (((1-methyl-1H-pyrazol-3- yl)sulfonyl)methyl)- 1,4,5,6,7,8-hexahydro-4aH- benzo[f]indazol-4a-yl)(5- (trifluoromethyl)pyridin-2- yl)methanone | 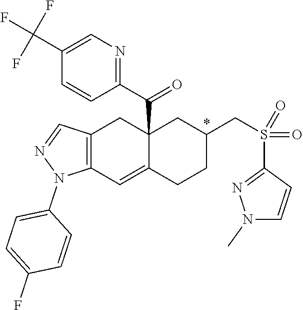 [SMILES: Cn1ccc(S(=O)(=O)CC2CCC3=Cc4c(cnn4-c4ccc(F)cc4)C[C@]3(C(=O)c3ccc(C(F)(F)F)cn3)C2)n1] | 600.3 |
| 361 | ((4aR)-1-(4-fluorophenyl)-6- (((1-methyl-1H-pyrazol-3- yl)sulfonyl)methyl)- 1,4,5,6,7,8-hexahydro-4aH- benzo[f]indazol-4a-yl)(5- (trifluoromethyl)thiazol-2- yl)methanone | 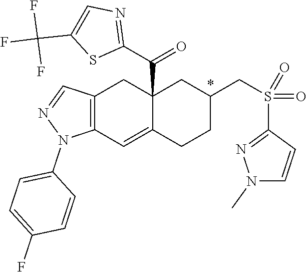 [SMILES: Cn1ccc(S(=O)(=O)CC2CCC3=Cc4c(cnn4-c4ccc(F)cc4)C[C@]3(C(=O)c3ncc(C(F)(F)F)s3)C2)n1] | 606.1 |
| 362 | ((4aR)-1-(4-fluorophenyl)-6- (((1-methyl-1H-pyrazol-3- yl)sulfonyl)methyl)- 1,4,5,6,7,8-hexahydro-4aH- benzo[f]indazol-4a-yl)(4- fluoropyridin-2-yl)methanone | 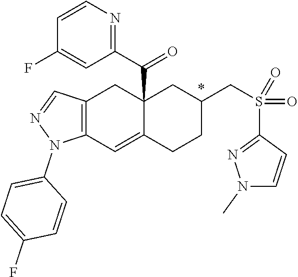 [SMILES: Cn1ccc(S(=O)(=O)CC2CCC3=Cc4c(cnn4-c4ccc(F)cc4)C[C@]3(C(=O)c3cc(F)ccn3)C2)n1] | 550.2 |
| 363 | N-((4aS,6S)-4a-(4- chloropicolinoyl)-1-(4- fluorophenyl)-4,4a,5,6,7,8- hexahydro-1H- benzo[f]indazol-6-yl)-N- cyclopropyl-1-methyl-1H- imidazole-4-sulfonamide | 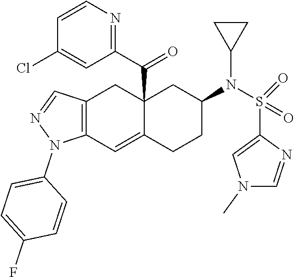 [SMILES: Cn1cnc(S(=O)(=O)N(C2CC2)[C@H]2CCC3=Cc4c(cnn4-c4ccc(F)cc4)C[C@]3(C(=O)c3cc(Cl)ccn3)C2)c1] | 607.2 |
| 364 | (4-chloropyridin-2-yl)((4aR)- 1-(4-fluorophenyl)-6-(((1- methyl-1H-pyrazol-3- yl)sulfonyl)methyl)- 1,4,5,6,7,8-hexahydro-4aH- benzo[f]indazol-4a- yl)methanone | 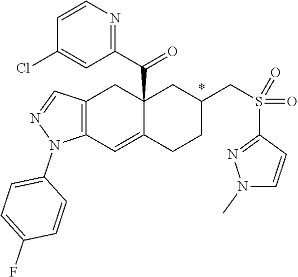 [SMILES: Cn1ccc(S(=O)(=O)CC2CCC3=Cc4c(cnn4-c4ccc(F)cc4)C[C@]3(C(=O)c3cc(Cl)ccn3)C2)n1] | 566.2 |
| 365 | ((4aS,6S)-1-(4-fluorophenyl)- 6-((1-methyl-1H-1,2,4-triazol- 3-yl)sulfonyl)-1,4,5,6,7,8- hexahydro-4aH- benzo[f]indazol-4a-yl)(4- (trifluoromethyl)pyridin-2- yl)methanone | 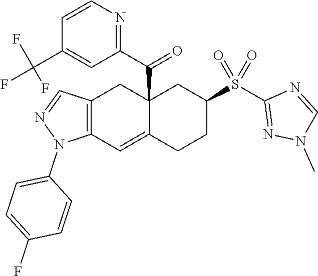 [SMILES: Cn1cnc(S(=O)(=O)[C@H]2CCC3=Cc4c(cnn4-c4ccc(F)cc4)C[C@]3(C(=O)c3cc(C(F)(F)F)ccn3)C2)n1] | 587.1 |
| 366 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- ((1s,3R)-3-hydroxy-3- (trifluoromethyl)cyclobutyl)-1- methyl-1H-pyrazole-3- sulfonamide | 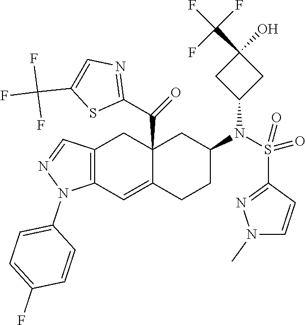 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](O)(C(F)(F)F)C2)n1] | 745.1 |
| 367 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-N-(2,2,2- trifluoroethyl)-1H-1,2,4- triazole-3-sulfonamide | 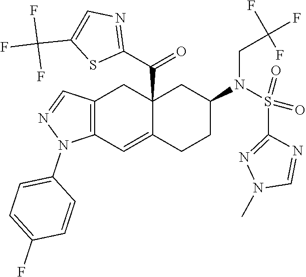 [SMILES: Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1] | 690.2 |
| 368 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N- ((1r,3S)-3-hydroxy-3- (trifluoromethyl)cyclobutyl)-1- methyl-1H-pyrazole-3- sulfonamide | 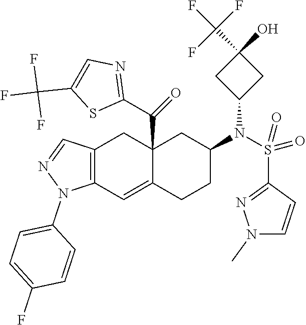 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@](O)(C(F)(F)F)C2)n1] | 745.1 |
| 369 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- ((1r,3S)-3-hydroxy-3- (trifluoromethyl)cyclobutyl)-1- methyl-1H-pyrazole-3- sulfonamide | 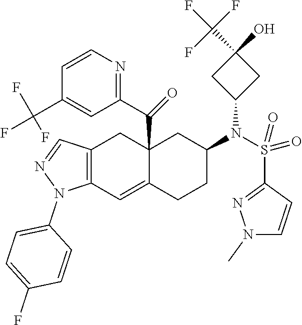 [SMILES: Cn1ccc(S(=O)(=O)N([C@H]2CCC3=Cc4c(cnn4-c4ccc(F)cc4)C[C@]3(C(=O)c3cc(C(F)(F)F)ccn3)C2)[C@H]2C[C@@](O)(C(F)(F)F)C2)n1] | 739.1 |
| 370 | 6-chloro-N-((4aS,6S)-4a-(4- chloropicolinoyl)-1-(4- fluorophenyl)-4,4a,5,6,7,8- hexahydro-1H- benzo[f]indazol-6-yl)-N-(3,3- difluorocyclobutyl)pyridine-3- sulfonamide | 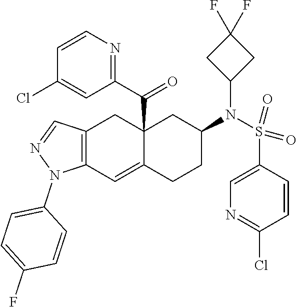 [SMILES: O=C(c1cc(Cl)ccn1)[C@]12Cc3cnn(-c4ccc(F)cc4)c3C=C1CC[C@H](N(C1CC(F)(F)C1)S(=O)(=O)c1ccc(Cl)nc1)C2] | 688.2 |
| 371 | N-((4aS,6S)-1-(4- fluorophenyl)-4a-(4- fluoropicolinoyl)-4,4a,5,6,7,8- hexahydro-1H- benzo[f]indazol-6-yl)-N- ((1s,3R)-3-methoxy-3- methylcyclobutyl)-1-methyl- 1H-pyrazole-3-sulfonamide | 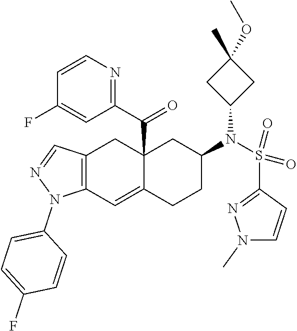 [SMILES: CO[C@]1(C)C[C@H](N([C@H]2CCC3=Cc4c(cnn4-c4ccc(F)cc4)C[C@]3(C(=O)c3cc(F)ccn3)C2)S(=O)(=O)c2ccn(C)n2)C1] | 649.2 |
| 372 | 4-fluoro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- methoxyethyl)benzenesulfon- amide | 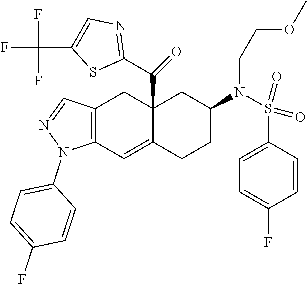 [SMILES: COCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1] | 679.1 |
| 373 | N-(2,2-difluoroethyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-1,2,3-triazole-4- sulfonamide | 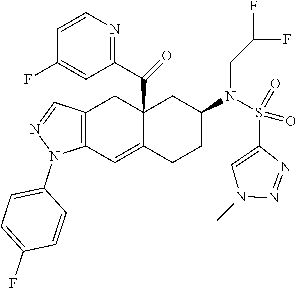 [SMILES: Cn1cc(S(=O)(=O)N(CC(F)F)[C@H]2CCC3=Cc4c(cnn4-c4ccc(F)cc4)C[C@]3(C(=O)c3cc(F)ccn3)C2)nn1] | 616.2 |
| 374 | ((4aS,6S)-1-(4-fluorophenyl)- 6-((1-methyl-1H-1,2,4-triazol- 3-yl)sulfonyl)-1,4,5,6,7,8- hexahydro-4aH- benzo[f]indazol-4a-yl)(5- (trifluoromethyl)pyridin-2- yl)methanone | 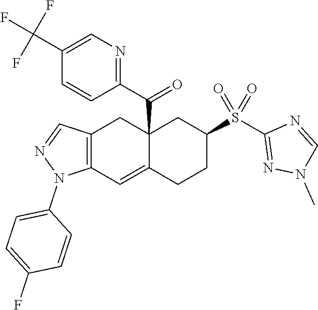 [SMILES: Cn1cnc(S(=O)(=O)[C@H]2CCC3=Cc4c(cnn4-c4ccc(F)cc4)C[C@]3(C(=O)c3ccc(C(F)(F)F)cn3)C2)n1] | 587 |
| 375 | ((4aR)-1-(4-fluorophenyl)-6- (((1-methyl-1H-pyrazol-4- yl)sulfonyl)methyl)- 1,4,5,6,7,8-hexahydro-4aH- benzo[f]indazol-4a-yl)(5- (trifluoromethyl)thiazol-2- yl)methanone | 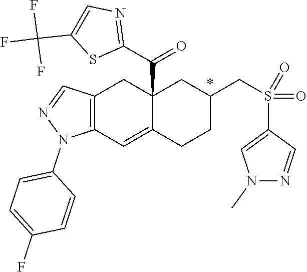 [SMILES: Cn1cc(S(=O)(=O)CC2CCC3=Cc4c(cnn4-c4ccc(F)cc4)C[C@]3(C(=O)c3ncc(C(F)(F)F)s3)C2)cn1] | 606.1 |
| 376 | N-ethyl-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-imidazole-5- sulfonamide | 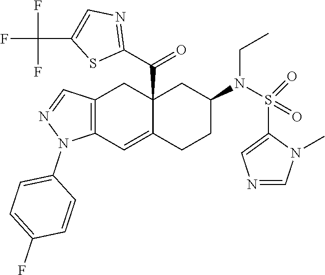 [SMILES: CCN([C@H]1CCC2=Cc3c(cnn3-c3ccc(F)cc3)C[C@]2(C(=O)c2ncc(C(F)(F)F)s2)C1)S(=O)(=O)c1cncn1C] | 635.2 |
| 377 | ((4aR)-1-(4-fluorophenyl)-6- (((1-methyl-1H-1,2,4-triazol-3- yl)sulfonyl)methyl)- 1,4,5,6,7,8-hexahydro-4aH- benzo[f]indazol-4a-yl)(4- (trifluoromethyl)pyridin-2- yl)methanone | 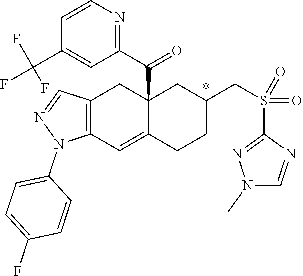 [SMILES: Cn1cnc(S(=O)(=O)CC2CCC3=Cc4c(cnn4-c4ccc(F)cc4)C[C@]3(C(=O)c3cc(C(F)(F)F)ccn3)C2)n1] | 601.2 |
| 378 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-1- methyl-N-(2,2,2- trifluoroethyl)-1H-1,2,4- triazole-3-sulfonamide | 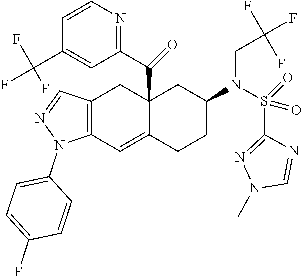 [SMILES: Cn1cnc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1] | 684.1 |
| 379 | ((4aR)-1-(4-fluorophenyl)-6- (((1-methyl-1H-1,2,4-triazol-3- yl)sulfonyl)methyl)- 1,4,5,6,7,8-hexahydro-4aH- benzo[f]indazol-4a-yl)(5- (trifluoromethyl)thiazol-2- yl)methanone | 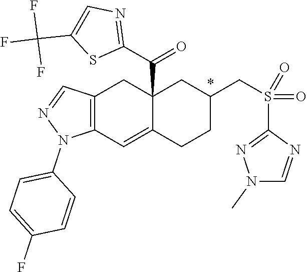 [SMILES: Cn1cnc(S(=O)(=O)CC2CCC3=Cc4c(cnn4-c4ccc(F)cc4)C[C@]3(C(=O)c3ncc(C(F)(F)F)s3)C2)n1] | 607.2 |
| 380 | N-(2-fluoroethyl)-N-((4aS,6S)- 1-(4-fluorophenyl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 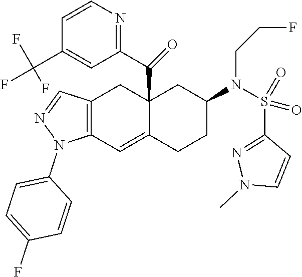 [SMILES: Cn1ccc(S(=O)(=O)N(CCF)[C@H]2CCC3=Cc4c(cnn4-c4ccc(F)cc4)C[C@]3(C(=O)c3cc(C(F)(F)F)ccn3)C2)n1] | 647.2 |
| 381 | N-((4aS,6S)-4a-(4- chloropicolinoyl)-1-(4- fluorophenyl)-4,4a,5,6,7,8- hexahydro-1H- benzo[f]indazol-6-yl)-N- ((1s,3R)-3-methoxy-3- methylcyclobutyl)-1-methyl- 1H-pyrazole-3-sulfonamide | 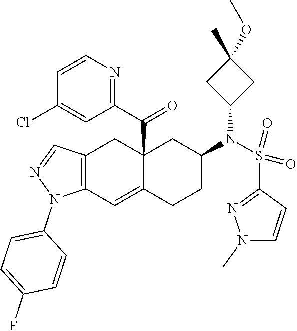 [SMILES: CO[C@]1(C)C[C@H](N([C@H]2CCC3=Cc4c(cnn4-c4ccc(F)cc4)C[C@]3(C(=O)c3cc(Cl)ccn3)C2)S(=O)(=O)c2ccn(C)n2)C1] | 665.2 |
| 382 | N-(2-fluoroethyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 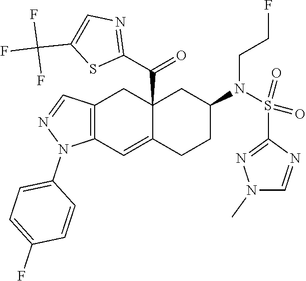 [SMILES: Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1] | 654.1 |
| 383 | N-(2,2-difluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-imidazole-4- sulfonamide | 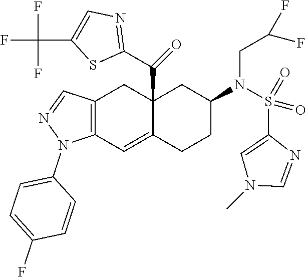 [SMILES: Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)c1] | 671.2 |
| 384 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N- ((1s,3R)-3-methoxy-3- methylcyclobutyl)-1-methyl- 1H-pyrazole-3-sulfonamide | 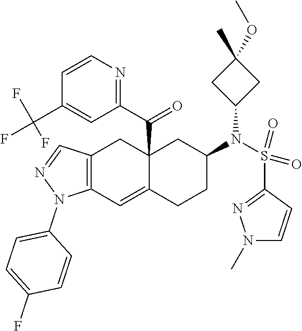 [SMILES: CO[C@]1(C)C[C@H](N([C@H]2CCC3=Cc4c(cnn4-c4ccc(F)cc4)C[C@]3(C(=O)c3cc(C(F)(F)F)ccn3)C2)S(=O)(=O)c2ccn(C)n2)C1] | 699.3 |
| 385 | N-(2-fluoroethyl)-N-((4aS,6S)- 1-(4-fluorophenyl)-4a-(4- (trifluoromethyl)picolinoyl)- 4,4a,5,6,7,8-hexahydro-1H- benzo[f]indazol-6-yl)-1- methyl-1H-1,2,4-triazole-3- sulfonamide | 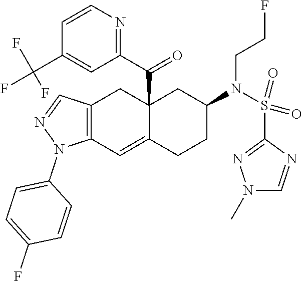 [SMILES: Cn1cnc(S(=O)(=O)N(CCF)[C@H]2CCC3=Cc4c(cnn4-c4ccc(F)cc4)C[C@]3(C(=O)c3cc(C(F)(F)F)ccn3)C2)n1] | 648.1 |
| 386 | 4-fluoro-N-(2-fluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6- yl)benzenesulfonamide | 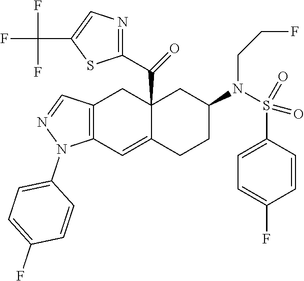 [SMILES: O=C(c1ncc(C(F)(F)F)s1)[C@]12Cc3cnn(-c4ccc(F)cc4)c3C=C1CC[C@H](N(CCF)S(=O)(=O)c1ccc(F)cc1)C2] | 667.1 |
| 387 | 4-fluoro-N-(2-fluoroethyl)-N- ((4aS,6S)-1-(4-fluorophenyl)- 4a-(4- (trifluoromethyl)picolinoyl)- 4,4a,5,6,7,8-hexahydro-1H- benzo[f]indazol-6- yl)benzenesulfonamide | 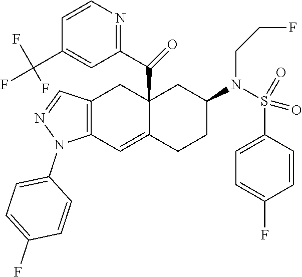 [SMILES: O=C(c1cc(C(F)(F)F)ccn1)[C@]12Cc3cnn(-c4ccc(F)cc4)c3C=C1CC[C@H](N(CCF)S(=O)(=O)c1ccc(F)cc1)C2] | 661.2 |
| 388 | N-(2-cyanoethyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 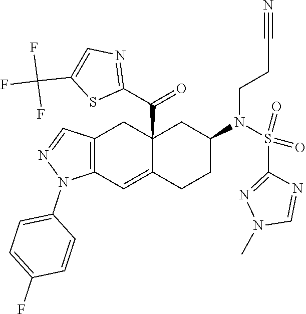 [SMILES: Cn1cnc(S(=O)(=O)N(CCC#N)[C@H]2CCC3=Cc4c(cnn4-c4ccc(F)cc4)C[C@]3(C(=O)c3ncc(C(F)(F)F)s3)C2)n1] | 661.1 |
| 389 | 4-fluoro-N-(2-fluoroethyl)-N- ((4aS,6S)-1-(4-fluorophenyl)- 4a-(4-fluoropicolinoyl)- 4,4a,5,6,7,8-hexahydro-1H- benzo[f]indazol-6- yl)benzenesulfonamide | 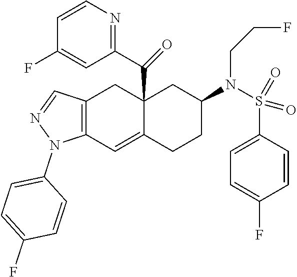 [SMILES: O=C(c1cc(F)ccn1)[C@]12Cc3cnn(-c4ccc(F)cc4)c3C=C1CC[C@H](N(CCF)S(=O)(=O)c1ccc(F)cc1)C2] | 611.2 |
| 390 | N-((4aS,6S)-4a-(4- chloropicolinoyl)-1-(4- fluorophenyl)-4,4a,5,6,7,8- hexahydro-1H- benzo[f]indazol-6-yl)-4-fluoro- N-(2- fluoroethyl)benzenesulfonamide | 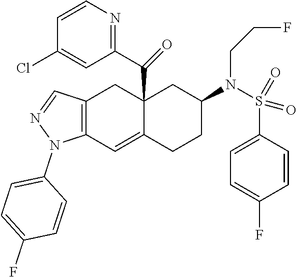 [SMILES: O=C(c1cc(Cl)ccn1)[C@]12Cc3cnn(-c4ccc(F)cc4)c3C=C1CC[C@H](N(CCF)S(=O)(=O)c1ccc(F)cc1)C2] | 627.2 |
| 391 | 4-fluoro-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2-(2- methoxyethoxy)ethyl)benzene- sulfonamide | 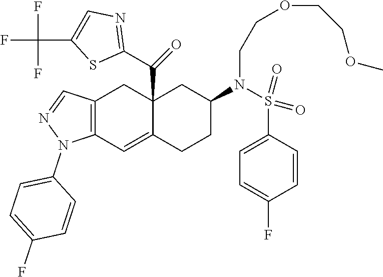 [SMILES: COCCOCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1] | 723.1 |
| 392 | N-(2-fluoroethyl)-N-((4aS,6S)- 1-(4-fluorophenyl)-4a-(4- fluoropicolinoyl)-4,4a,5,6,7,8- hexahydro-1H- benzo[f]indazol-6-yl)-1- methyl-1H-1,2,4-triazole-3- sulfonamide | 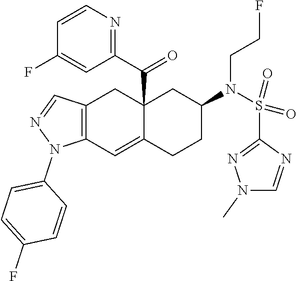 [SMILES: Cn1cnc(S(=O)(=O)N(CCF)[C@H]2CCC3=Cc4c(cnn4-c4ccc(F)cc4)C[C@]3(C(=O)c3cc(F)ccn3)C2)n1] | 598.2 |
| 393 | N-((4aS,6S)-4a-(4- chloropicolinoyl)-1-(4- fluorophenyl)-4,4a,5,6,7,8- hexahydro-1H- benzo[f]indazol-6-yl)-N-(2- fluoroethyl)-1-methyl-1H- 1,2,4-triazole-3-sulfonamide | 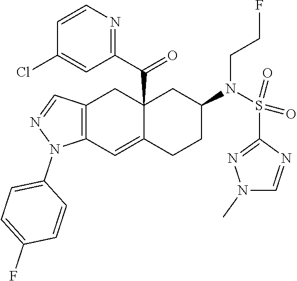 [SMILES: Cn1cnc(S(=O)(=O)N(CCF)[C@H]2CCC3=Cc4c(cnn4-c4ccc(F)cc4)C[C@]3(C(=O)c3cc(Cl)ccn3)C2)n1] | 614.2 |
| 394 | N-(2-fluoroethyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,3-triazole-4- sulfonamide | 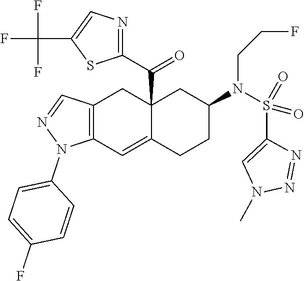 [SMILES: Cn1cc(S(=O)(=O)N(CCF)[C@H]2CCC3=Cc4c(cnn4-c4ccc(F)cc4)C[C@]3(C(=O)c3ncc(C(F)(F)F)s3)C2)nn1] | 654.2 |
| 395 | N-(2-fluoroethyl)-N-((4aS,6S)- 1-(4-fluorophenyl)-4a-(4- (trifluoromethyl)picolinoyl)- 4,4a,5,6,7,8-hexahydro-1H- benzo[f]indazol-6-yl)-1- methyl-1H-1,2,3-triazole-4- sulfonamide | 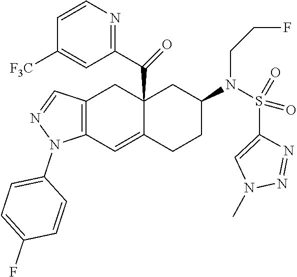 [SMILES: Cn1cc(S(=O)(=O)N(CCF)[C@H]2CCC3=Cc4c(cnn4-c4ccc(F)cc4)C[C@]3(C(=O)c3cc(C(F)(F)F)ccn3)C2)nn1] | 648.2 |
| 396 | N-(2-fluoroethyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1- methyl-1H-pyrazole-4- sulfonamide | 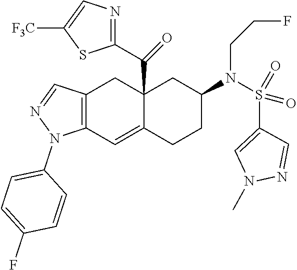 [SMILES: Cn1cc(S(=O)(=O)N(CCF)[C@H]2CCC3=Cc4c(cnn4-c4ccc(F)cc4)C[C@]3(C(=O)c3ncc(C(F)(F)F)s3)C2)cn1] | 653.2 |
| 397 | N-(2-fluoroethyl)-N-((4aS,6S)- 1-(4-fluorophenyl)-4a-(4- fluoropicolinoyl)-4,4a,5,6,7,8- hexahydro-1H- benzo[f]indazol-6-yl)-1- methyl-1H-pyrazole-3- sulfonamide | 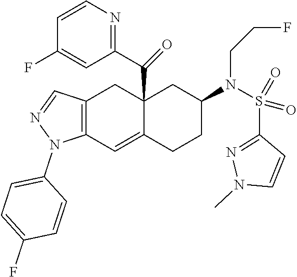 [SMILES: Cn1ccc(S(=O)(=O)N(CCF)[C@H]2CCC3=Cc4c(cnn4-c4ccc(F)cc4)C[C@]3(C(=O)c3cc(F)ccn3)C2)n1] | 597.2 |
| 398 | N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-N-(2- methoxyethyl)-1-methyl-1H- 1,2,4-triazole-3-sulfonamide | 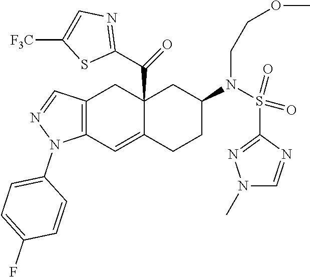 [SMILES: COCCN([C@H]1CCC2=Cc3c(cnn3-c3ccc(F)cc3)C[C@]2(C(=O)c2ncc(C(F)(F)F)s2)C1)S(=O)(=O)c1ncn(C)n1] | 666.2 |
| 399 | N-cyclopropyl-N-((4aS,6S)-1- (6-fluoropyridin-3-yl)-4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 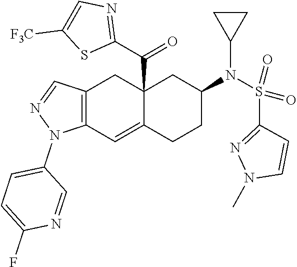 [SMILES: Cn1ccc(S(=O)(=O)N(C2CC2)[C@H]2CCC3=Cc4c(cnn4-c4ccc(F)nc4)C[C@]3(C(=O)c3ncc(C(F)(F)F)s3)C2)n1] | 648.2 |
| 400 | tert-butyl N-((4aS,6S)-1-(4- fluorophenyl)-4a-(4- (trifluoromethyl)picolinoyl)- 4,4a,5,6,7,8-hexahydro-1H- benzo[f]indazol-6-yl)-N-((1- methyl-1H-pyrazol-3- yl)sulfonyl)glycinate | 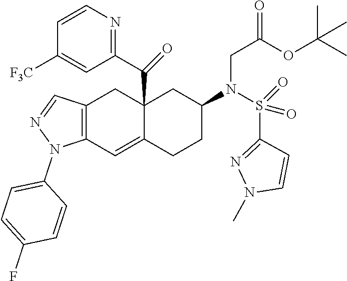 [SMILES: Cn1ccc(S(=O)(=O)N(CC(=O)OC(C)(C)C)[C@H]2CCC3=Cc4c(cnn4-c4ccc(F)cc4)C[C@]3(C(=O)c3cc(C(F)(F)F)ccn3)C2)n1] | 715.2 |
| 401 | N-((4aS,6S)-1-(4- fluorophenyl)-4a-(4- (trifluoromethyl)picolinoyl)- 4,4a,5,6,7,8-hexahydro-1H- benzo[f]indazol-6-yl)-N-((1- methyl-1H-pyrazol-3- yl)sulfonyl)glycine | 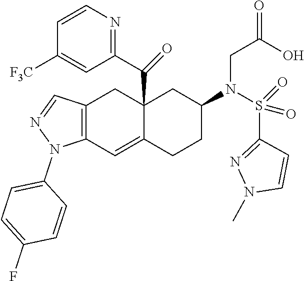 [SMILES: Cn1ccc(S(=O)(=O)N(CC(=O)O)[C@H]2CCC3=Cc4c(cnn4-c4ccc(F)cc4)C[C@]3(C(=O)c3cc(C(F)(F)F)ccn3)C2)n1] | 659.2 |
| 402 | N-cyclopropyl-N-((4aS,6S)-1- (6-fluoropyridin-3-yl)-4a-(4- (trifluoromethyl)picolinoyl)- 4,4a,5,6,7,8-hexahydro-1H- benzo[f]indazol-6-yl)-1- methyl-1H-pyrazole-3- sulfonamide | 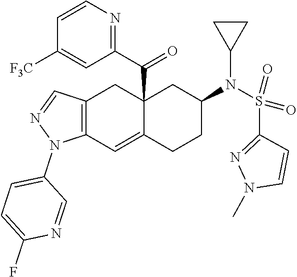 [SMILES: Cn1ccc(S(=O)(=O)N(C2CC2)[C@H]2CCC3=Cc4c(cnn4-c4ccc(F)nc4)C[C@]3(C(=O)c3cc(C(F)(F)F)ccn3)C2)n1] | 642.2 |
| 403 | N-((4aS,6S)-4a-(4- chloropicolinoyl)-1-(4- fluorophenyl)-4,4a,5,6,7,8- hexahydro-1H- benzo[f]indazol-6-yl)-N-(2- fluoroethyl)-1-methyl-1H- pyrazole-3-sulfonamide | 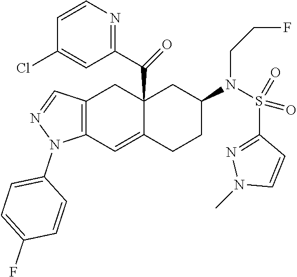 [SMILES: Cn1ccc(S(=O)(=O)N(CCF)[C@H]2CCC3=Cc4c(cnn4-c4ccc(F)cc4)C[C@]3(C(=O)c3cc(Cl)ccn3)C2)n1] | 613.3 |
| 404 | 4-fluoro-N-((4aS,6S)-1-(4- fluorophenyl)-4a-(4- (trifluoromethyl)picolinoyl)- 4,4a,5,6,7,8-hexahydro-1H- benzo[f]indazol-6-yl)-N-(2-(2- methoxyethoxy)ethyl)benzene- sulfonamide | 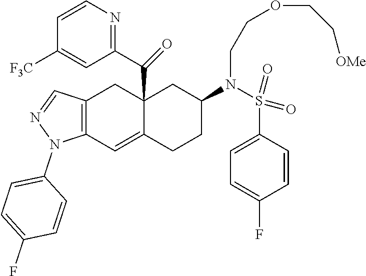 [SMILES: COCCOCCN([C@H]1CCC2=Cc3c(cnn3-c3ccc(F)cc3)C[C@]2(C(=O)c2cc(C(F)(F)F)ccn2)C1)S(=O)(=O)c1ccc(F)cc1] | 717.2 |
| 405 | N-(2,2-difluoroethyl)-4-fluoro- N-((4aS,6S)-1-(4- fluorophenyl)-4a-(5- (trifluoromethyl)thiazole-2- carbonyl)-4,4a,5,6,7,8- hexahydro-1H- benzo[f]indazol-6- yl)benzenesulfonamide | 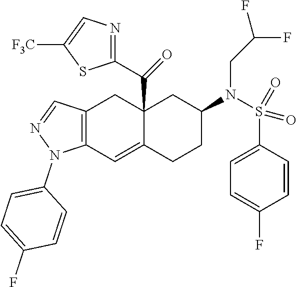 [SMILES: O=C(c1ncc(C(F)(F)F)s1)[C@]12Cc3cnn(-c4ccc(F)cc4)c3C=C1CC[C@H](N(CC(F)F)S(=O)(=O)c1ccc(F)cc1)C2] | 685.1 |
| 406 | N-(3,3-difluorocyclobutyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-4- sulfonamide | 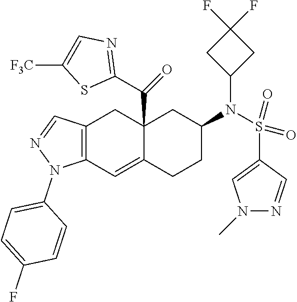 [SMILES: Cn1cc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)cn1] | 697.1 |
| 407 | N-(2,2-difluoroethyl)-4-fluoro- N-((4aS,6S)-1-(4- fluorophenyl)-4a-(4- (trifluoromethyl)picolinoyl)- 4,4a,5,6,7,8-hexahydro-1H- benzo[f]indazol-6- yl)benzenesulfonamide | 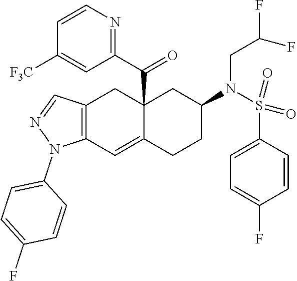 [SMILES: O=C(c1cc(C(F)(F)F)ccn1)[C@]12Cc3cnn(-c4ccc(F)cc4)c3C=C1CC[C@H](N(CC(F)F)S(=O)(=O)c1ccc(F)cc1)C2] | 679.1 |
| 408 | N-((1s,3R)-3- cyanocyclobutyl)-N-((4aS,6S)- 1-(4-fluorophenyl)-4a-(5- (trifluoromethyl)thiazole-2- carbonyl)-4,4a,5,6,7,8- hexahydro-1H- benzo[f]indazol-6-yl)-1- methyl-1H-pyrazole-3- sulfonamide | 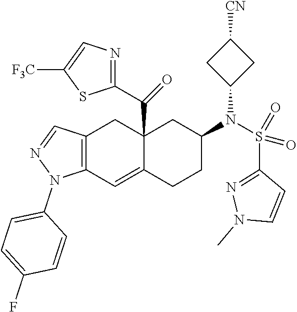 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@H](C#N)C2)n1] | 686.2 |
| 409 | N-(cyclopropylmethyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-1,2,4-triazole-3- sulfonamide | 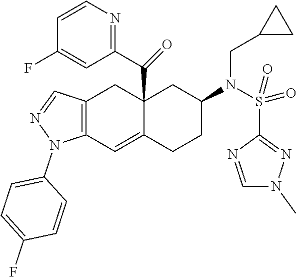 [SMILES: Cn1cnc(S(=O)(=O)N(CC2CC2)[C@H]2CCC3=Cc4c(cnn4-c4ccc(F)cc4)C[C@]3(C(=O)c3cc(F)ccn3)C2)n1] | 606.2 |
| 410 | N-((4aS,6S)-4a-(4- chloropicolinoyl)-1-(4- fluorophenyl)-4,4a,5,6,7,8- hexahydro-1H- benzo[f]indazol-6-yl)-N- (cyclopropylmethyl)-1-methyl- 1H-1,2,4-triazole-3- sulfonamide | 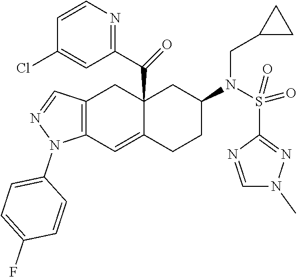 [SMILES: Cn1cnc(S(=O)(=O)N(CC2CC2)[C@H]2CCC3=Cc4c(cnn4-c4ccc(F)cc4)C[C@]3(C(=O)c3cc(Cl)ccn3)C2)n1] | 622.2 |
| 411 | N-(3,3-difluorocyclobutyl)-N- ((4aS,6S)-1-(4-fluorophenyl)- 4a-(4- (trifluoromethyl)picolinoyl)- 4,4a,5,6,7,8-hexahydro-1H- benzo[f]indazol-6-yl)-1- methyl-1H-pyrazole-4- sulfonamide | 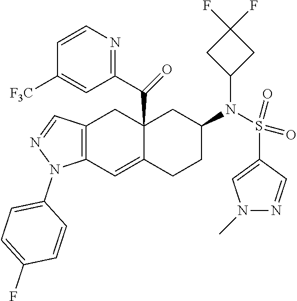 [SMILES: Cn1cc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)cn1] | 691.2 |
| 412 | N-(1,3-dimethoxypropan-2- yl)-N-((4aS,6S)-1-(4- fluorophenyl)-4a-(5- (trifluoromethyl)thiazole-2- carbonyl)-4,4a,5,6,7,8- hexahydro-1H- benzo[f]indazol-6-yl)-1- methyl-1H-1,2,4-triazole-3- sulfonamide | 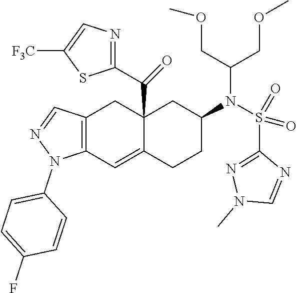 [SMILES: COCC(COC)N([C@H]1CCC2=Cc3c(cnn3-c3ccc(F)cc3)C[C@]2(C(=O)c2ncc(C(F)(F)F)s2)C1)S(=O)(=O)c1ncn(C)n1] | 710.2 |
| 413 | N-(3,3-difluorocyclobutyl)-N- ((4aS,6S)-1-(4-fluorophenyl)- 4a-(4-fluoropicolinoyl)- 4,4a,5,6,7,8-hexahydro-1H- benzo[f]indazol-6-yl)-1- methyl-1H-pyrazole-4- sulfonamide | 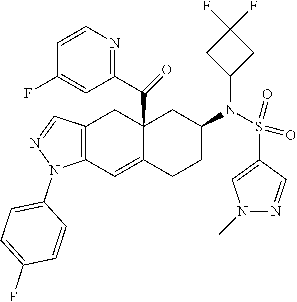 [SMILES: Cn1cc(S(=O)(=O)N(C2CC(F)(F)C2)[C@H]2CCC3=Cc4c(cnn4-c4ccc(F)cc4)C[C@]3(C(=O)c3cc(F)ccn3)C2)cn1] | 641.3 |
| 414 | N-(2,2-difluoroethyl)-N- ((4aS,6S)-1-(4-fluorophenyl)- 4a-(5- (trifluoromethyl)thiazole-2- carbonyl)-4,4a,5,6,7,8- hexahydro-1H- benzo[f]indazol-6-yl)-1- methyl-1H-pyrazole-4- sulfonamide | 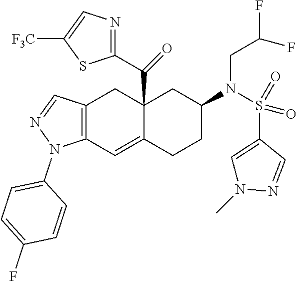 [SMILES: Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)cn1] | 671 |

*Single enantiomer of undetermined stereochemistry

### II. Biological Evaluation

### Example A: In Vitro GR Luciferase Reporter Assay

Cell Line: CHO-K1-GR-MMTV-Luc reporter cells

Culture Media: DMEM (with phenol red)+10% FBS

Assay Media: DMEM (without phenol red)+10% CSS

Culture CHO-K1I-GR-MMTV-Luc reporter cells in 15 cm plates in Culture Media at conditions less than 90% confluence.

Prepare 200×DMSO 1:5 serial dilutions of control and test compounds in 96-well non-sterile V bottom plate in DMSO, 8serial dilutions for each compound.

Prepare 5× Assay Media diluted compound serial dilutions in 96-well non-sterile V bottom plate: Add 97.5 uL/well of Assay Media into 96-well then add 2.5 ul of 200× concentration of compounds and mix well.

Seed cells for Antagonist Assay: 1.5×106 CHO-K1-GR-MMTV-Luc reporter cells were seeded in a Corning 3707 flat clear bottom 384-well white TC plate in 20 ul of Assay Media containing 12.5 nM Dexamethasone (final concentration=10 nM).

Add compounds: 5 ul of assay media diluted compounds were added to appropriate wells and followed a quick spin (1000 rpm, 10 sec) to bring media and cells to the bottom of plate. The plates were covered with SealMate film to avoid evaporation and placed in 37° C. incubator for approximately 18-24 hours.

Read plates: Equilibrate appropriate amount of Promega OneGlo luciferase reagent to room temperature. Remove the plates from incubator and add 25 uL of OneGlo reagent/well by multiple channel pipette and read the plates with Tecan F500 luminometer within 3 minutes.

The ability of the compounds disclosed herein to inhibit GR activity was quantified and the respective IC₅₀ value was determined. Table 1and Table 2provide the cellular IC₅₀ values of compounds disclosed herein.

TABLE 1

| | GR IC₅₀ | | GR IC₅₀ | | GR IC₅₀ |
|---|---|---|---|---|---|
| Ex. | (nM) | Ex. | (nM) | Ex. | (nM) |
| 1 | A | 2 | A | 3 | A |
| 4 | A | 5 | A | 6 | A |
| 7 | A | 8 | A | 9 | A |
| 10 | A | 11 | A | 12 | B |
| 13 | B | 14 | A | 15 | A |
| 16 | A | 17 | A | 18 | A |
| 19 | A | 20 | A | 21 | A |
| 22 | A | 23 | A | 24 | A |
| 25 | A | 26 | A | 27 | B |
| 28 | A | 29 | B | 30 | A |
| 31 | A | 32 | A | 33 | A |
| 34 | A | 35 | A | 36 | A |
| 37 | A | 38 | A | 39 | A |
| 40 | A | 41 | A | 42 | A |
| 43 | A | 44 | A | 45 | A |
| 46 | A | 47 | A | 48 | A |
| 49 | A | 50 | A | 51 | A |
| 52 | A | 53 | A | 54 | A |
| 55 | A | 56 | A | 57 | A |
| 58 | A | 59 | A | 60 | A |
| 61 | A | 62 | A | 63 | A |
| 64 | A | 65 | A | 66 | A |
| 67 | A | 68 | A | 69 | A |
| 70 | A | 71 | A | 72 | A |
| 73 | A | 74 | A | 75 | A |
| 76 | A | 77 | A | 78 | A |
| 79 | A | 80 | A | 81 | A |
| 82 | A | 83 | A | 84 | A |
| 85 | A | 86 | A | 87 | A |
| 88 | A | 89 | A | 90 | A |
| 91 | A | 92 | B | 93 | A |
| 94 | A | 95 | A | 96 | A |
| 97 | A | 98 | A | 99 | A |
| 100 | B | 101 | B | 102 | A |
| 103 | A | 104 | B | 105 | A |
| 106 | A | 107 | A | 108 | A |
| 109 | A | 110 | A | | |

A = IC₅₀ is less than or equal to 100 nM;
B = IC₅₀ is greater than 100 nM and less than 1 μM;

TABLE 2

| Ex. | GR IC₅₀ (nM) |
|---|---|
| 111 | A |
| 112 | A |
| 113 | A |
| 114 | A |
| 115 | A |
| 116 | A |
| 117 | B |
| 118 | A |
| 119 | A |
| 120 | A |
| 121 | A |
| 122 | A |
| 123 | A |
| 124 | A |
| 125 | A |
| 126 | A |
| 127 | A |
| 128 | A |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 160 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 201 | A |
| 202 | A |
| 203 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | A |
| 285 | A |
| 286 | A |
| 287 | A |
| 288 | A |
| 289 | A |
| 290 | A |
| 291 | B |
| 292 | A |
| 293 | A |
| 294 | A |
| 295 | A |
| 296 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | A |
| 315 | A |
| 316 | A |
| 317 | A |
| 318 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | A |
| 345 | A |
| 346 | A |
| 347 | A |
| 348 | A |
| 349 | A |
| 350 | A |
| 351 | A |
| 352 | A |
| 353 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 373 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 389 | A |
| 390 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | B |
| 402 | A |
| 403 | A |
| 404 | A |
| 405 | A |
| 406 | A |
| 407 | A |
| 408 | A |
| 409 | A |
| 410 | A |
| 411 | A |

A = IC₅₀ is less than or equal to 100 nM;
B = IC₅₀ is greater than 100 nM; and less than 1 μM;

### Example B: CYP Inhibition Assay

Objective: To assess the % inhibition of test compounds against CYP2C8 enzyme in human liver microsomes.

Pooled human microsomes were obtained from Sekisui XenoTech, KS, and were used for in vitro assessment of compound inhibitory potential of the major drug metabolizing human CYP enzymes.

Test compounds were dissolved in DMSO as 10 mM stock solutions and diluted to 200 M in acetonitrile as working solutions. The total concentrations of DMSO and acetonitrile were 0.02% and 0.5%, respectively, in the assay. The selective probe substrate paclitaxel at the final concentration of 10 M and the reference control compound quercetin at 2 M were used for the CYP2C8 inhibition assay.

The assay was performed in duplicate in a 96-well plate. The reaction mixture (200 L) containing a final concentration of 0.1 mg/mL pooled human liver microsomes, 50 mM potassium phosphate, pH 7.4 buffer, was re-warmed at 37° C. for 5 minutes. The reaction was initiated with NADPH solution which was at 1 mM final concentration, and carried out for 5 minutes at 37° C. It was terminated by the addition of 3-fold volume of quench solution (1:1 acetonitrile:methanol with 0.1% formic acid and the internal standard of tolbutamide). The sample was vortexed, centrifuged at 4,000 rpm for 15 min at 4° C. The incubation in the presence of vehicle (0.02% DMSO and 0.5% acetonitrile) was used as the enzyme activity control. The analyses of metabolite 6α-hydroxypaclitaxel in supernatant was performed with LC-MS/MS method (Sciex API 4500 Qtrap coupled to a Shimadzu LC-20AD HPLC).

The CYP enzyme activity was determined based on the formation of 6a-hydroxypaclitaxel metabolite. The % inhibition was calculated using this equation: % Inhibition=100×(1−A_{inactivator}/A_{vehicle}), where A_{inactivator} is the CYP enzyme activity in the presence of test compound, and A_{vehicle} is the CYP activity in the presence of vehicle.

The ability of some exemplary compounds disclosed herein to inhibit CYP2C8 activity was quantified. Table 3 provides the % inhibition against CYP2C8 enzyme.

TABLE 3

| | % Inhibition of CYP2C8 |
|---|---|
| Ex. | (Paclitaxel) |
| 4 | C |
| 5 | B |
| 52 | D |
| 55 | A |
| 56 | D |
| 69 | A |
| 81 | B |
| 88 | B |
| 90 | C |
| 91 | C |
| 93 | D |
| 94 | C |
| 98 | B |
| 99 | B |
| 102 | D |
| 103 | B |
| 104 | C |
| 111 | B |
| 112 | D |
| 113 | A |
| 114 | A |
| 118 | A |
| 122 | A |
| 131 | C |
| 134 | D |
| 138 | C |
| 140 | D |
| 142 | C |
| 144 | D |
| 153 | C |
| 155 | B |
| 156 | D |
| 160 | C |
| 164 | C |
| 166 | B |
| 170 | A |
| 176 | B |
| 177 | A |
| 178 | B |
| 179 | B |
| 182 | C |
| 183 | B |
| 184 | B |
| 186 | C |
| 189 | C |
| 191 | B |
| 193 | C |
| 194 | C |
| 195 | B |
| 196 | C |
| 198 | B |
| 199 | D |
| 201 | A |
| 202 | B |
| 203 | B |
| 204 | B |
| 206 | B |
| 210 | D |
| 211 | B |
| 212 | B |
| 214 | A |
| 215 | C |
| 216 | B |
| 217 | B |
| 219 | B |
| 221 | C |
| 222 | C |
| 223 | B |
| 224 | B |
| 225 | C |
| 226 | C |
| 227 | B |
| 228 | C |
| 229 | C |
| 230 | C |
| 231 | B |
| 232 | B |
| 233 | D |
| 234 | D |
| 236 | C |
| 241 | D |
| 242 | D |
| 243 | C |
| 244 | D |
| 245 | C |
| 246 | B |
| 247 | C |
| 248 | A |
| 250 | C |
| 251 | C |
| 254 | C |
| 255 | C |
| 257 | C |
| 258 | C |
| 259 | B |
| 260 | C |
| 261 | C |
| 262 | C |
| 263 | D |
| 265 | C |
| 266 | D |
| 267 | C |
| 268 | C |
| 269 | D |
| 270 | A |
| 272 | A |
| 273 | A |
| 277 | A |
| 279 | C |
| 280 | B |
| 282 | B |
| 283 | A |
| 286 | D |
| 287 | B |
| 288 | A |
| 289 | A |
| 290 | B |
| 293 | A |
| 294 | A |
| 295 | C |
| 296 | C |
| 298 | c |
| 299 | A |
| 300 | c |
| 305 | B |
| 306 | A |
| 307 | B |
| 308 | B |
| 309 | A |
| 310 | B |
| 311 | A |
| 312 | B |
| 313 | B |
| 315 | B |
| 319 | C |
| 320 | B |
| 321 | A |
| 322 | B |
| 324 | B |
| 325 | A |
| 326 | C |
| 327 | B |
| 328 | B |
| 329 | B |
| 330 | B |
| 331 | A |
| 332 | A |
| 333 | A |
| 334 | C |
| 335 | A |
| 337 | C |
| 338 | D |
| 339 | B |
| 341 | B |
| 342 | B |
| 344 | B |
| 346 | B |
| 347 | B |
| 348 | B |
| 349 | B |
| 350 | B |
| 354 | B |
| 355 | A |
| 356 | B |
| 357 | A |
| 358 | B |
| 359 | B |
| 363 | B |
| 366 | A |
| 367 | B |
| 368 | B |
| 371 | B |
| 373 | C |
| 374 | A |
| 377 | B |

A = % is less than or equal to 25;
B = % is greater than 25 nM and less or equal to 50;
C = % is greater than 50 and less or equal to 75;
D = % is greater than 75 and less or equal to 90.

As a reference, (R)-(1-(4-fluorophenyl)-6-((1-methyl-H-pyrazol-4-yl)sulfonyl)-1,4,5,6,7,8-hexahydro-4aH-pyrazolo[3,4-g]isoquinolin-4a-yl)(4-(trifluoromethyl)pyridin-2-yl)methanone showed a 92% inhibition in this assay.

### Example C: In Vitro PR Luciferase Reporter Assay

Cell Line: CHO-KI-PR-MMTV-Luc reporter cells

Culture Media: DMEM (with phenol red)+1000FBS

Assay Media: DMEM (without phenol red)+1000CSS

Culture CHO-Kl-PR-MMTV-Luc reporter cells in 15 cm plates in Culture Media at conditions less than 90% confluence.

Prepared 200×DMSO 1:8 serial dilutions of control and test compounds in 96-well non-sterile V bottom plate in DMSO, 8 serial dilutions for each compound.

Prepared 5× Assay Media diluted compound serial dilutions in 96-well non-sterile V bottom plate: Add 97.5 μL/well of Assay Media into 96-well then add 2.5 μl of 200× concentration of compounds and mix well.

Seeded cells for Antagonist Assay: 1.5×10⁶ CHO-KI-PR-MMTV-Luc reporter cells were seeded in a Corning 3707 flat clear bottom 384-well white TC plate in 20 ul of Assay Media containing 25 nM Progesterone (final concentration=20 nM).

Added compounds: 5 μl of assay media diluted compounds were added to appropriate wells and followed a quick spin (1000 rpm, 10 sec) to bring media and cells to the bottom of plate. The plates were covered with SealMate film to avoid evaporation and placed in 37° C. incubator for approximately 18-24 hours.

Read plates: Equilibrated appropriate amount of Promega One-Glo luciferase reagent to room temperature. Removed the plates from incubator and added 25 μL of One-Glo luciferase reagent/well by multiple channel pipette and read the plates with Tecan F500 luminometer within 3 minutes.

The ability of exemplary compounds disclosed herein to inhibit PR activity was quantified and the respective IC₅₀ value was determined. Table 4 provides the cellular IC₅₀ values of exemplary compounds disclosed herein.

TABLE 4

| Ex. | PR antagonism IC₅₀ (nM) |
|---|---|
| 5 | >5,000 |
| 88 | >5,000 |
| 98 | 3,470 |
| 103 | >5,000 |
| 111 | >5,000 |
| 113 | >5,000 |
| 118 | >5,000 |
| 138 | >5,000 |
| 177 | >5,000 |
| 179 | >5,000 |
| 186 | >5,000 |
| 211 | >5,000 |
| 227 | >5,000 |
| 232 | >5,000 |
| 248 | >5,000 |
| 270 | 3,600 |
| 277 | >5,000 |
| 305 | >5,000 |
| 306 | >5,000 |

1. A compound having the structure of Formula (I), or a pharmaceutically acceptable salt, solvate, or stereoisomer thereof: 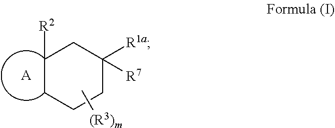
wherein: 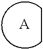
is 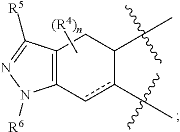 [symbol]
is a single bond or a double bond;
R^{1a} is —NR¹⁶C(O)R¹⁷, —NR¹⁶S(O)₂R¹⁷, —S(O)₂NR¹⁸R¹⁹, —C(R²⁰)₂S(O)₂R¹⁷, —C(O)NR¹⁸R¹⁹, —S(O)₂CH₂R¹⁷, or —S(O)₂R¹;
R² is hydrogen, halogen, alkyl, alkenyl, —CN, —OR⁸, —NR⁸R⁹, cycloalkyl, heterocycloalkyl, aryl, heteroaryl, —C(O)R¹¹, —C(O)OR^{B}, —OC(O)R¹¹, —C(O)NR⁸R⁹, —NR^{B}C(O)R¹¹, —NR⁸C(O)OR⁹, —NR¹⁰C(O)NR⁸R⁹, —OC(O)NR⁸R⁹, —S(O)₂R¹¹, —S(O)R¹¹, —SR⁸, —S(O)₂NR⁸R⁹, —NR⁸S(O)₂R¹¹, or —NR¹⁰S(O)₂NR⁸R⁹, wherein alkyl, alkenyl, cycloalkyl, heterocycloalkyl, aryl, and heteroaryl are optionally substituted with one, two, or three R^{12b};
each R³ and each R⁴ is independently halogen or alkyl;
R⁵ is hydrogen, alkyl, or haloalkyl;
R⁶ is aryl, heteroaryl, cycloalkyl, or heterocycloalkyl, wherein aryl, heteroaryl, cycloalkyl, and heterocycloalkyl are optionally substituted with one, two, or three R^{12c};
R⁷ is hydrogen, halogen, —CN, alkyl, haloalkyl, heteroalkyl, alkenyl, —OR⁸, —NR⁸R⁹, cycloalkyl, or heterocycloalkyl;
each R⁸ and each R⁹ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12d};
or R⁸ and R⁹ are taken together with the atom to which they are attached to form a heterocycloalkyl optionally substituted with one, two, or three R^{12d};
R¹⁰ is hydrogen or alkyl;
R¹¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12e};
each R^{12a}, R^{12b}, R^{12c}, R^{12d}, R^{12e}, R^{12f}, and R^{12e} is independently selected from halogen, —CN, alkyl, haloalkyl, —OR¹³, -alkyl-OR¹³, —NR¹³R¹⁴, -alkyl-NR¹³R¹⁴, cycloalkyl, -alkyl-cycloalkyl, heterocycloalkyl, aryl, heteroaryl, —C(O)R¹⁵, —C(O)OR¹³, —C(O)NR¹³R¹⁴, —S(O)₂R¹⁵, —SR¹³, and —S(O)₂NR¹³R¹⁴; wherein cycloalkyl, heterocycloalkyl, aryl, and heteroaryl are optionally substituted with one, two, or three groups selected from halogen, alkyl, and haloalkyl;
each R¹³ and each R¹⁴ is independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
or R¹³ and R¹⁴ are taken together with the atom to which they are attached to form a heterocycloalkyl optionally substituted with one, two, or three groups selected from halogen, alkyl, and haloalkyl;
each R¹⁵ is independently alkyl, cycloalkyl, heterocycloalkyl, aryl, or heteroaryl, wherein alkyl, cycloalkyl, heterocycloalkyl, aryl, and heteroaryl are optionally substituted with one, two, or three groups selected from halogen, alkyl, and haloalkyl;
R¹⁶ is hydrogen, alkyl, cycloalkyl, or heterocycloalkyl, wherein alkyl, cycloalkyl, and heterocycloalkyl are optionally substituted with one, two, or three groups selected from halogen, alkyl, haloalkyl, alkoxy, heteroalkyl, cycloalkyl, heterocycloalkyl, —CN, —OR¹³, —NR¹³R¹⁴, —C(O)R¹⁵, —C(O)OR¹³, —C(O)NR¹³R¹⁴, —NR¹³C(O)R¹⁵, —NR¹³C(O)OR¹³, —NR¹³C(O)NR¹³R¹⁴, —S(O)₂R¹⁵, —S(O)R¹⁵, —SR¹³, —S(O)₂NR¹³R¹⁴, —NR¹³S(O)₂R¹⁵, and —NR¹³S(O)₂NR¹³R¹⁴;
R¹⁷ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12f};
R¹⁸ and R¹⁹ is each independently hydrogen, alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12g};
R²⁰ is hydrogen, halogen, —CN, alkyl, haloalkyl, heteroalkyl, alkenyl, —OR⁸, —NR⁸R⁹, cycloalkyl, or heterocycloalkyl;
R¹ is alkyl, cycloalkyl, heterocycloalkyl, aryl, or heteroaryl, wherein alkyl, cycloalkyl, heterocycloalkyl, aryl, and heteroaryl are optionally substituted with one, two, or three R^{12a};
m is 0, 1, 2, 3, or 4; and
n is 0, 1, 2, or 3.

2. (canceled)

3. (canceled)

4. The compound of claim 1, or a pharmaceutically acceptable salt, solvate, or stereoisomer thereof, wherein R^{1a} is —NR¹⁶S(O)₂R¹⁷ or —S(O)₂R¹.

5. (canceled)

6. (canceled)

7. The compound of claim 1, or a pharmaceutically acceptable salt, solvate, or stereoisomer thereof, wherein R¹⁶ is C₁₋₆alkyl or C₃₋₈cycloalkyl, wherein C₁₋₆alkyl and C₃₋₈cycloalkyl are optionally substituted with one, two, or three groups selected from halogen, C₁₋₆alkyl, C₁₋₆haloalkyl, C₁₋₆heteroalkyl, C₃₋₈cycloalkyl, C₂₋₉heterocycloalkyl, —CN, —OR¹³, —C(O)OR¹³, and —S(O)₂R¹⁵.

8. The compound of claim 1, or a pharmaceutically acceptable salt, solvate, or stereoisomer thereof, wherein R¹⁶ is unsubstituted C₁₋₆alkyl or unsubstituted C₃₋₈cycloalkyl.

9. (canceled)

10. (canceled)

11. (canceled)

12. The compound of claim 1, or a pharmaceutically acceptable salt, solvate, or stereoisomer thereof, wherein R²⁰ is hydrogen, C₁₋₆alkyl, or C₃₋₈cycloalkyl.

13. The compound of claim 1, or a pharmaceutically acceptable salt, solvate, or stereoisomer thereof, wherein R¹⁷ is C₆₋₁₀aryl or C₂₋₉heteroaryl, and the C₆₋₁₀aryl and C₂₋₉heteroaryl are optionally substituted with one, two, or three R^{12f}.

14. (canceled)

15. (canceled)

16. (canceled)

17. (canceled)

18. (canceled)

19. (canceled)

20. The compound of claim 1, or a pharmaceutically acceptable salt, solvate, or stereoisomer thereof, wherein R¹⁸ and R¹⁹ is each independently hydrogen, C₁₋₆alkyl, C₆₋₁₀aryl, or C₂₋₉heteroaryl, wherein C₁₋₆alkyl, C₆₋₁₀aryl, and C₂₋₉heteroaryl are optionally substituted with one, two, or three R^{12g}.

21. (canceled)

22. (canceled)

23. (canceled)

24. The compound of claim 1, or a pharmaceutically acceptable salt or solvate thereof, wherein R¹ is C₆₋₁₀aryl or C₂₋₉heteroaryl, and the C₆₋₁₀aryl and C₂₋₉heteroaryl are optionally substituted with one, two, or three R^{12a}.

25. (canceled)

26. (canceled)

27. (canceled)

28. (canceled)

29. (canceled)

30. The compound of claim 1, or a pharmaceutically acceptable salt, solvate, or stereoisomer thereof, wherein R⁶ is phenyl optionally substituted with one, two, or three R^{12c}.

31. (canceled)

32. (canceled)

33. (canceled)

34. The compound of claim 1, or a pharmaceutically acceptable salt, solvate, or stereoisomer thereof, wherein R² is C₁₋₆alkyl, C₂₋₆alkenyl, C₃₋₈cycloalkyl, or —C(O)R¹¹, wherein C₁₋₆alkyl, C₂₋₆alkenyl, and C₃₋₈cycloalkyl are optionally substituted with one, two, or three R^{12b}.

35. (canceled)

36. (canceled)

37. (canceled)

38. The compound of claim 1, or a pharmaceutically acceptable salt, solvate, or stereoisomer thereof, wherein R² is —C(O)R¹¹.

39. The compound of claim 38, or a pharmaceutically acceptable salt, solvate, or stereoisomer thereof, wherein R¹¹ is C₆₋₁₀aryl or C₂₋₉heteroaryl, wherein C₆₋₁₀aryl and C₂₋₉heteroaryl are optionally substituted with one, two, or three R^{12e}.

40. (canceled)

41. (canceled)

42. (canceled)

43. (canceled)

44. The compound of claim 1, or a pharmaceutically acceptable salt, solvate, or stereoisomer thereof, wherein R⁷ is hydrogen, halogen, or C₁₋₆ alkyl.

45. (canceled)

46. The compound of claim 1, or a pharmaceutically acceptable salt, solvate, or stereoisomer thereof, wherein m is 0.

47. The compound of claim 1, or a pharmaceutically acceptable salt, solvate, or stereoisomer thereof, wherein n is 0.

48. The compound of claim 1, or a pharmaceutically acceptable salt, solvate, or stereoisomer thereof, wherein R⁵ is hydrogen.

49. (canceled)

50. The compound of claim 1 or a pharmaceutically acceptable salt, solvate, or stereoisomer thereof, wherein the compound has the structure of Formula (Ib):

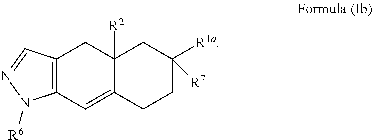

51. (canceled)

52. (canceled)

53. (canceled)

54. (canceled)

55. (canceled)

56. (canceled)

57. (canceled)

58. A compound selected from: 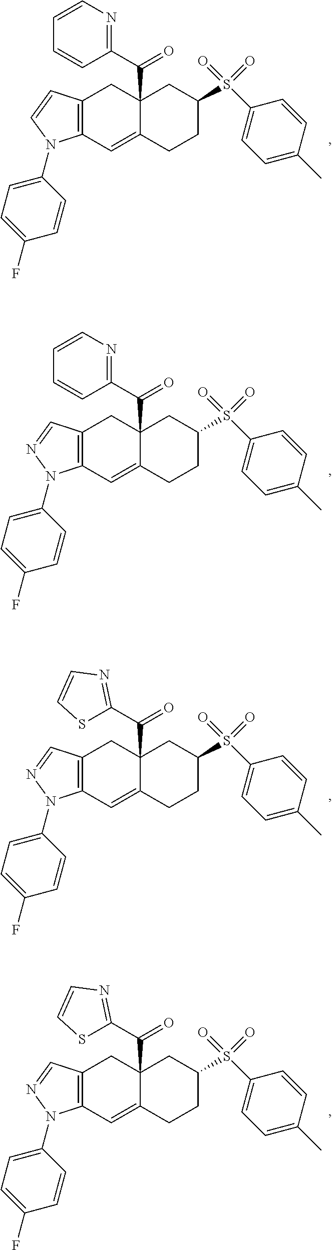 [SMILES: Cc1ccc(S(=O)(=O)[C@H]2CCC3=Cc4c(ccn4-c4ccc(F)cc4)C[C@]3(C(=O)c3ccccn3)C2)cc1 ; Cc1ccc(S(=O)(=O)[C@@H]2CCC3=Cc4c(cnn4-c4ccc(F)cc4)C[C@]3(C(=O)c3ccccn3)C2)cc1 ; Cc1ccc(S(=O)(=O)[C@H]2CCC3=Cc4c(cnn4-c4ccc(F)cc4)C[C@]3(C(=O)c3nccs3)C2)cc1 ; Cc1ccc(S(=O)(=O)[C@@H]2CCC3=Cc4c(cnn4-c4ccc(F)cc4)C[C@]3(C(=O)c3nccs3)C2)cc1] 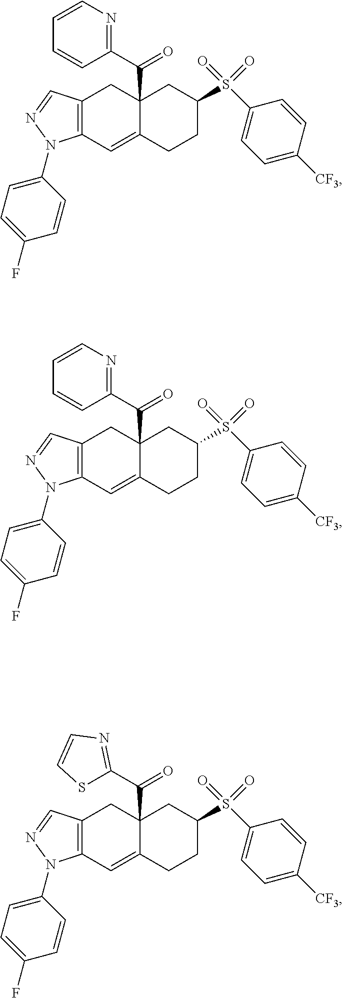 [SMILES: O=C(c1ccccn1)[C@]12Cc3cnn(-c4ccc(F)cc4)c3C=C1CC[C@H](S(=O)(=O)c1ccc(C(F)(F)F)cc1)C2 ; O=C(c1ccccn1)[C@]12Cc3cnn(-c4ccc(F)cc4)c3C=C1CC[C@@H](S(=O)(=O)c1ccc(C(F)(F)F)cc1)C2 ; O=C(c1nccs1)[C@]12Cc3cnn(-c4ccc(F)cc4)c3C=C1CC[C@H](S(=O)(=O)c1ccc(C(F)(F)F)cc1)C2] 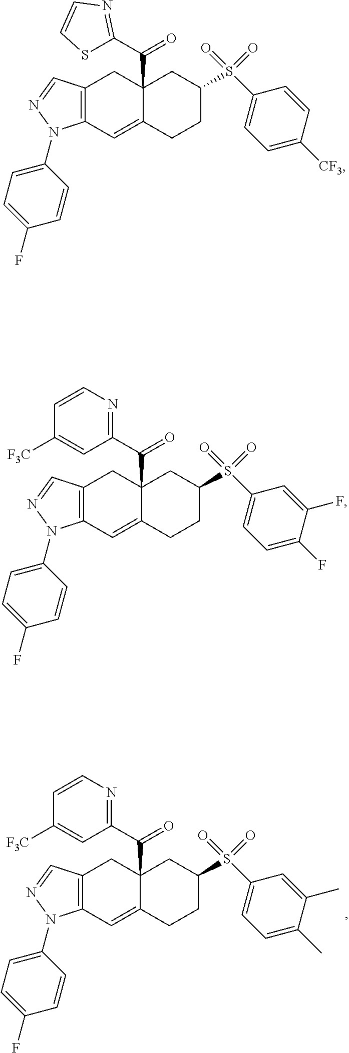 [SMILES: O=C(c1nccs1)[C@]12Cc3cnn(-c4ccc(F)cc4)c3C=C1CC[C@@H](S(=O)(=O)c1ccc(C(F)(F)F)cc1)C2 ; O=C(c1cc(C(F)(F)F)ccn1)[C@]12Cc3cnn(-c4ccc(F)cc4)c3C=C1CC[C@H](S(=O)(=O)c1ccc(F)c(F)c1)C2 ; Cc1ccc(S(=O)(=O)[C@H]2CCC3=Cc4c(cnn4-c4ccc(F)cc4)C[C@]3(C(=O)c3cc(C(F)(F)F)ccn3)C2)cc1C] 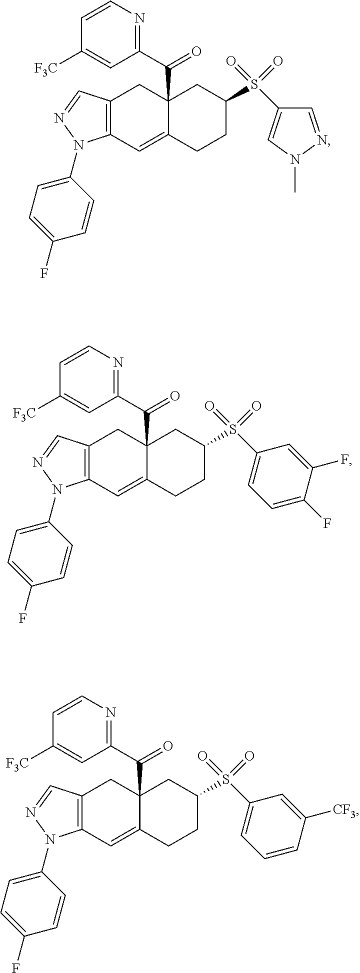 [SMILES: Cn1cc(S(=O)(=O)[C@H]2CCC3=Cc4c(cnn4-c4ccc(F)cc4)C[C@]3(C(=O)c3cc(C(F)(F)F)ccn3)C2)cn1 ; O=C(c1cc(C(F)(F)F)ccn1)[C@]12Cc3cnn(-c4ccc(F)cc4)c3C=C1CC[C@@H](S(=O)(=O)c1ccc(F)c(F)c1)C2 ; O=C(c1cc(C(F)(F)F)ccn1)[C@]12Cc3cnn(-c4ccc(F)cc4)c3C=C1CC[C@@H](S(=O)(=O)c1cccc(C(F)(F)F)c1)C2] 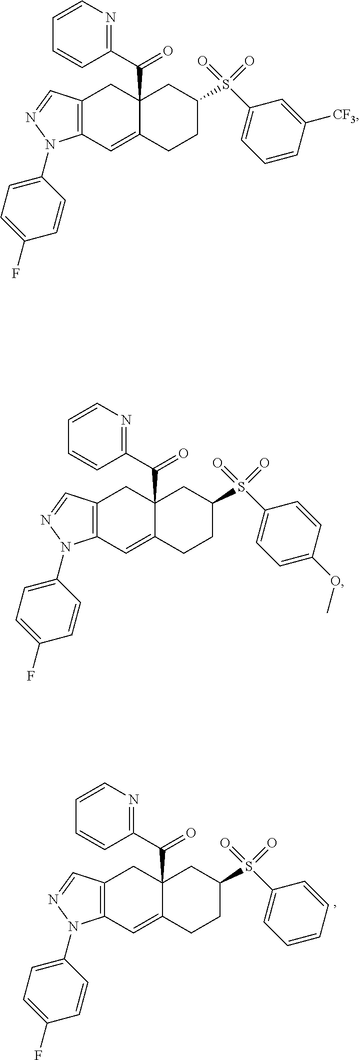 [SMILES: O=C(c1ccccn1)[C@]12Cc3cnn(-c4ccc(F)cc4)c3C=C1CC[C@@H](S(=O)(=O)c1cccc(C(F)(F)F)c1)C2 ; COc1ccc(S(=O)(=O)[C@H]2CCC3=Cc4c(cnn4-c4ccc(F)cc4)C[C@]3(C(=O)c3ccccn3)C2)cc1 ; O=C(c1ccccn1)[C@]12Cc3cnn(-c4ccc(F)cc4)c3C=C1CC[C@H](S(=O)(=O)c1ccccc1)C2] 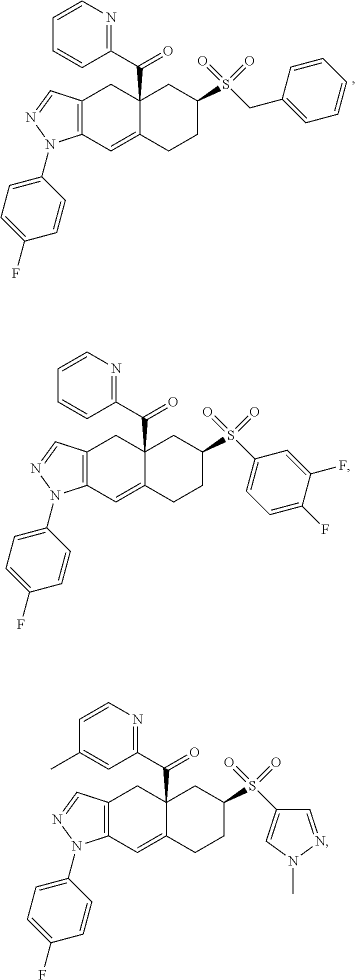 [SMILES: O=C(c1ccccn1)[C@]12Cc3cnn(-c4ccc(F)cc4)c3C=C1CC[C@H](S(=O)(=O)Cc1ccccc1)C2 ; O=C(c1ccccn1)[C@]12Cc3cnn(-c4ccc(F)cc4)c3C=C1CC[C@H](S(=O)(=O)c1ccc(F)c(F)c1)C2 ; Cc1ccnc(C(=O)[C@]23Cc4cnn(-c5ccc(F)cc5)c4C=C2CC[C@H](S(=O)(=O)c2cnn(C)c2)C3)c1] 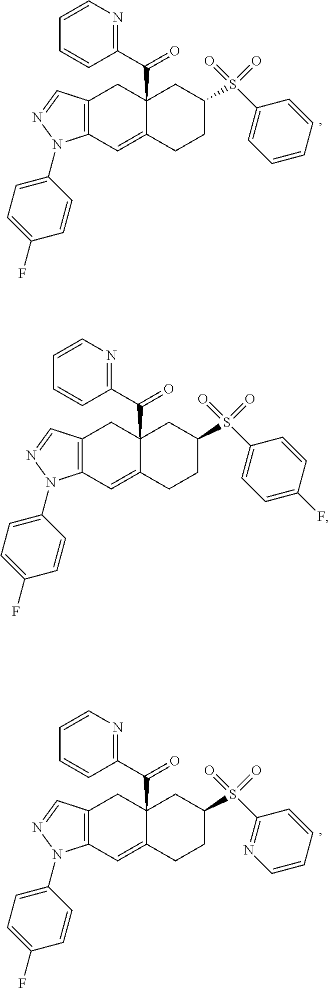 [SMILES: O=C(c1ccccn1)[C@]12Cc3cnn(-c4ccc(F)cc4)c3C=C1CC[C@@H](S(=O)(=O)c1ccccc1)C2 ; O=C(c1ccccn1)[C@]12Cc3cnn(-c4ccc(F)cc4)c3C=C1CC[C@H](S(=O)(=O)c1ccc(F)cc1)C2 ; O=C(c1ccccn1)[C@]12Cc3cnn(-c4ccc(F)cc4)c3C=C1CC[C@H](S(=O)(=O)c1ccccn1)C2] 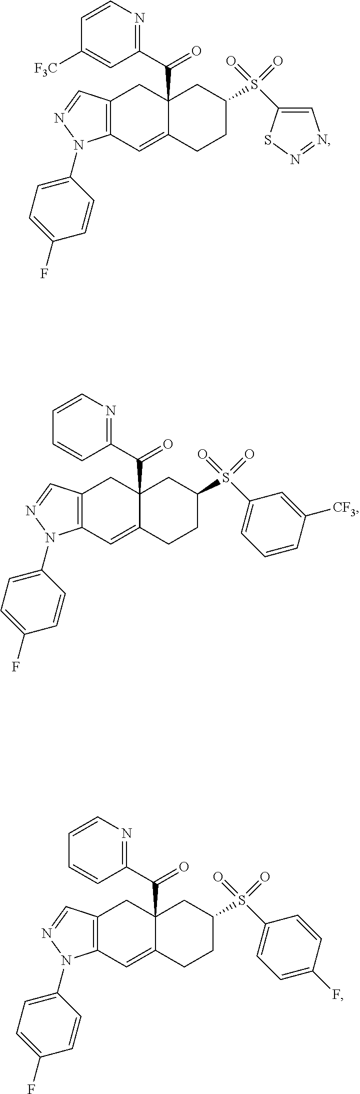 [SMILES: O=C(c1cc(C(F)(F)F)ccn1)[C@]12Cc3cnn(-c4ccc(F)cc4)c3C=C1CC[C@@H](S(=O)(=O)c1cnns1)C2 ; O=C(c1ccccn1)[C@]12Cc3cnn(-c4ccc(F)cc4)c3C=C1CC[C@H](S(=O)(=O)c1cccc(C(F)(F)F)c1)C2 ; O=C(c1ccccn1)[C@]12Cc3cnn(-c4ccc(F)cc4)c3C=C1CC[C@@H](S(=O)(=O)c1ccc(F)cc1)C2] 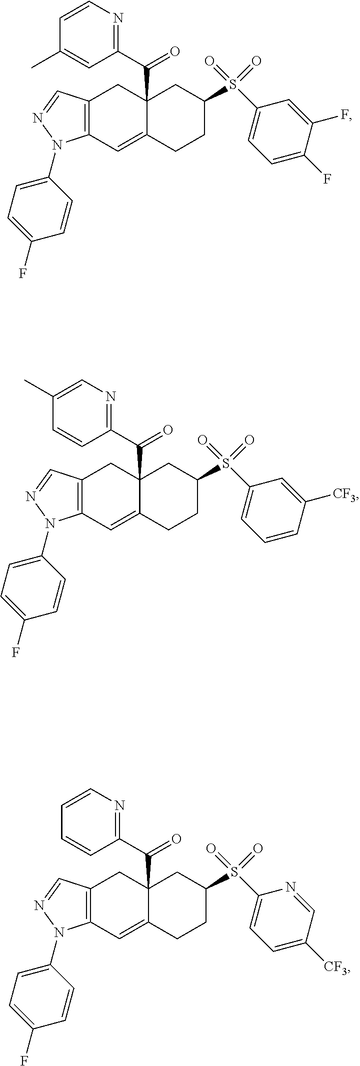 [SMILES: Cc1ccnc(C(=O)[C@]23Cc4cnn(-c5ccc(F)cc5)c4C=C2CC[C@H](S(=O)(=O)c2ccc(F)c(F)c2)C3)c1 ; Cc1ccc(C(=O)[C@]23Cc4cnn(-c5ccc(F)cc5)c4C=C2CC[C@H](S(=O)(=O)c2cccc(C(F)(F)F)c2)C3)nc1 ; O=C(c1ccccn1)[C@]12Cc3cnn(-c4ccc(F)cc4)c3C=C1CC[C@H](S(=O)(=O)c1ccc(C(F)(F)F)cn1)C2] 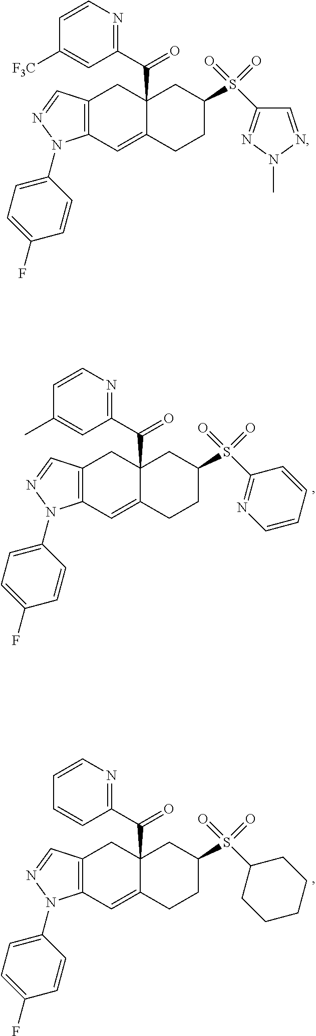 [SMILES: Cn1ncc(S(=O)(=O)[C@H]2CCC3=Cc4c(cnn4-c4ccc(F)cc4)C[C@]3(C(=O)c3cc(C(F)(F)F)ccn3)C2)n1 ; Cc1ccnc(C(=O)[C@]23Cc4cnn(-c5ccc(F)cc5)c4C=C2CC[C@H](S(=O)(=O)c2ccccn2)C3)c1 ; O=C(c1ccccn1)[C@]12Cc3cnn(-c4ccc(F)cc4)c3C=C1CC[C@H](S(=O)(=O)C1CCCCC1)C2] 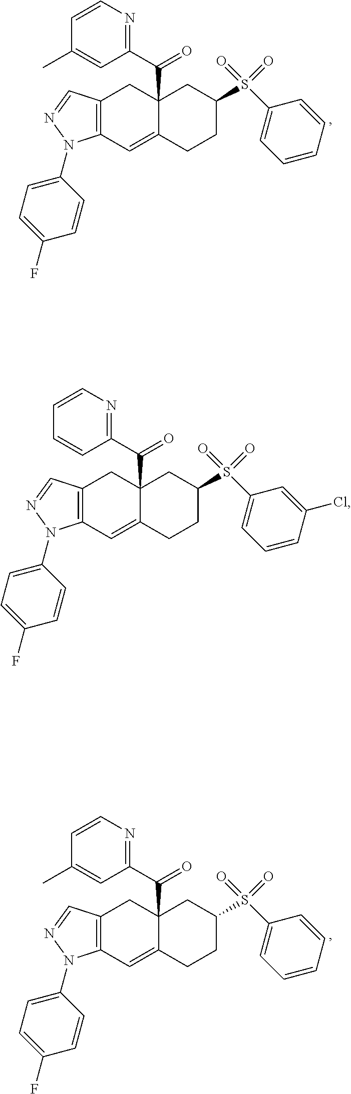 [SMILES: Cc1ccnc(C(=O)[C@]23Cc4cnn(-c5ccc(F)cc5)c4C=C2CC[C@H](S(=O)(=O)c2ccccc2)C3)c1 ; O=C(c1ccccn1)[C@]12Cc3cnn(-c4ccc(F)cc4)c3C=C1CC[C@H](S(=O)(=O)c1cccc(Cl)c1)C2 ; Cc1ccnc(C(=O)[C@]23Cc4cnn(-c5ccc(F)cc5)c4C=C2CC[C@@H](S(=O)(=O)c2ccccc2)C3)c1] 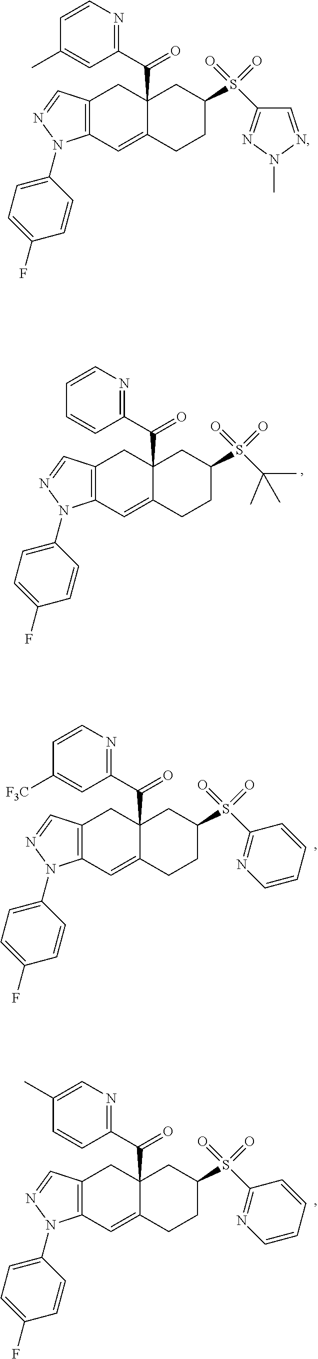 [SMILES: Cc1ccnc(C(=O)[C@]23Cc4cnn(-c5ccc(F)cc5)c4C=C2CC[C@H](S(=O)(=O)c2cnn(C)n2)C3)c1 ; CC(C)(C)S(=O)(=O)[C@H]1CCC2=Cc3c(cnn3-c3ccc(F)cc3)C[C@]2(C(=O)c2ccccn2)C1 ; O=C(c1cc(C(F)(F)F)ccn1)[C@]12Cc3cnn(-c4ccc(F)cc4)c3C=C1CC[C@H](S(=O)(=O)c1ccccn1)C2 ; Cc1ccc(C(=O)[C@]23Cc4cnn(-c5ccc(F)cc5)c4C=C2CC[C@H](S(=O)(=O)c2ccccn2)C3)nc1] 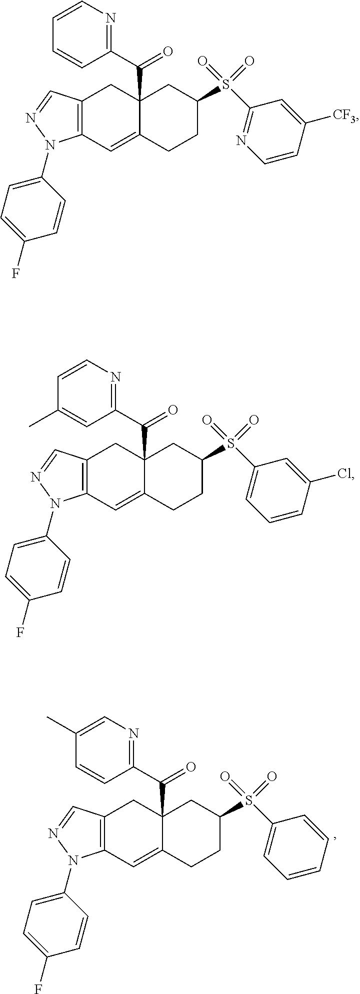 [SMILES: O=C(c1ccccn1)[C@]12Cc3cnn(-c4ccc(F)cc4)c3C=C1CC[C@H](S(=O)(=O)c1cc(C(F)(F)F)ccn1)C2 ; Cc1ccnc(C(=O)[C@]23Cc4cnn(-c5ccc(F)cc5)c4C=C2CC[C@H](S(=O)(=O)c2cccc(Cl)c2)C3)c1 ; Cc1ccc(C(=O)[C@]23Cc4cnn(-c5ccc(F)cc5)c4C=C2CC[C@H](S(=O)(=O)c2ccccc2)C3)nc1] 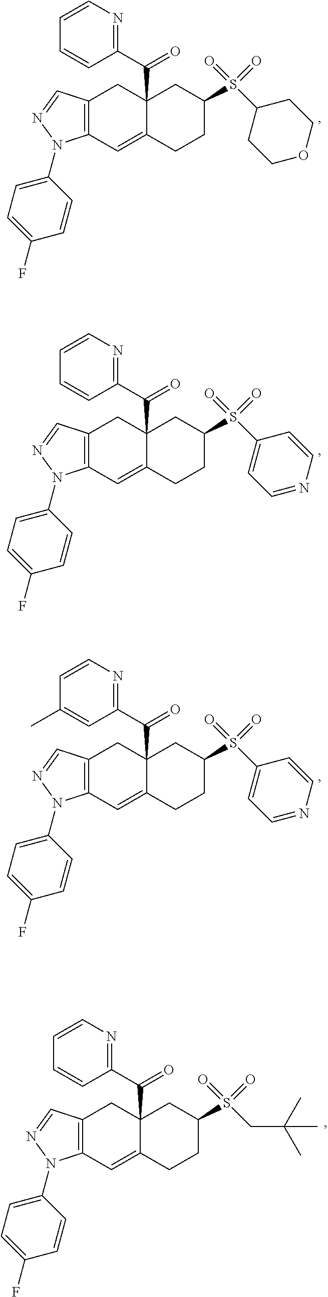 [SMILES: O=C(c1ccccn1)[C@]12Cc3cnn(-c4ccc(F)cc4)c3C=C1CC[C@H](S(=O)(=O)C1CCOCC1)C2 ; O=C(c1ccccn1)[C@]12Cc3cnn(-c4ccc(F)cc4)c3C=C1CC[C@H](S(=O)(=O)c1ccncc1)C2 ; Cc1ccnc(C(=O)[C@]23Cc4cnn(-c5ccc(F)cc5)c4C=C2CC[C@H](S(=O)(=O)c2ccncc2)C3)c1 ; CC(C)(C)CS(=O)(=O)[C@H]1CCC2=Cc3c(cnn3-c3ccc(F)cc3)C[C@]2(C(=O)c2ccccn2)C1] 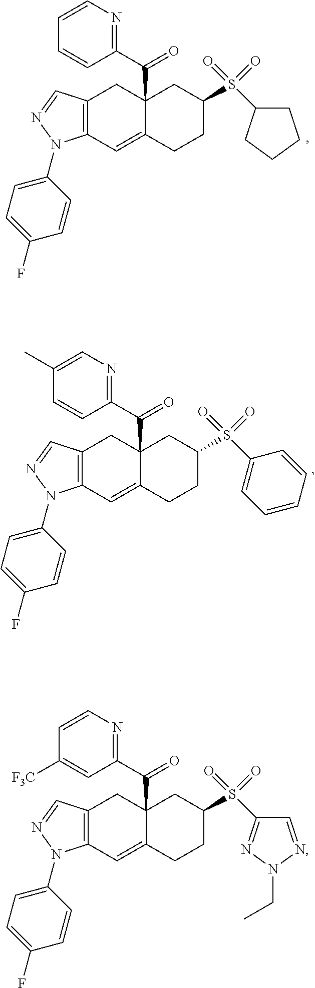 [SMILES: O=C(c1ccccn1)[C@]12Cc3cnn(-c4ccc(F)cc4)c3C=C1CC[C@H](S(=O)(=O)C1CCCC1)C2 ; Cc1ccc(C(=O)[C@]23Cc4cnn(-c5ccc(F)cc5)c4C=C2CC[C@@H](S(=O)(=O)c2ccccc2)C3)nc1 ; CCn1ncc(S(=O)(=O)[C@H]2CCC3=Cc4c(cnn4-c4ccc(F)cc4)C[C@]3(C(=O)c3cc(C(F)(F)F)ccn3)C2)n1] 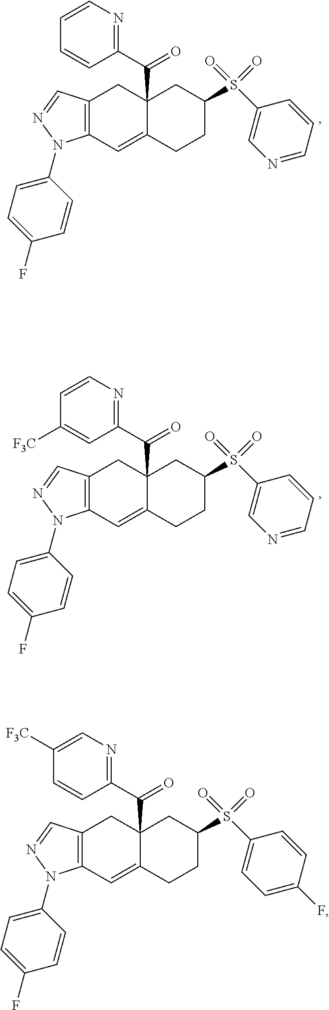 [SMILES: O=C(c1ccccn1)[C@]12Cc3cnn(-c4ccc(F)cc4)c3C=C1CC[C@H](S(=O)(=O)c1cccnc1)C2 ; O=C(c1cc(C(F)(F)F)ccn1)[C@]12Cc3cnn(-c4ccc(F)cc4)c3C=C1CC[C@H](S(=O)(=O)c1cccnc1)C2 ; O=C(c1ccc(C(F)(F)F)cn1)[C@]12Cc3cnn(-c4ccc(F)cc4)c3C=C1CC[C@H](S(=O)(=O)c1ccc(F)cc1)C2] 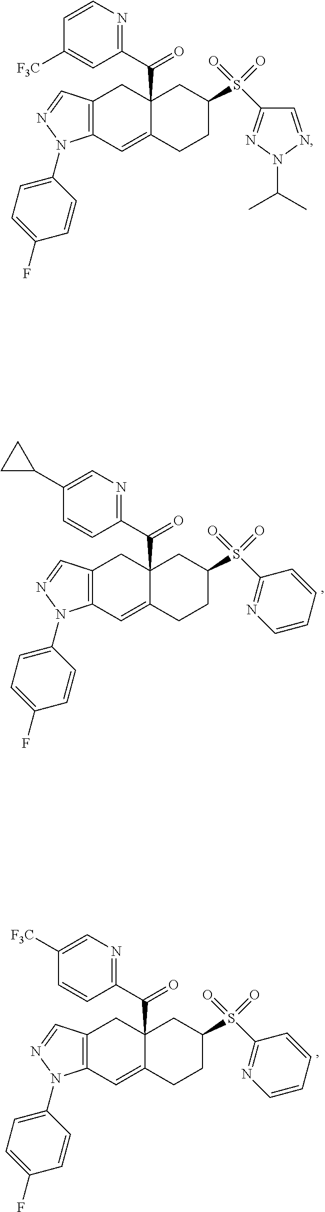 [SMILES: CC(C)n1ncc(S(=O)(=O)[C@H]2CCC3=Cc4c(cnn4-c4ccc(F)cc4)C[C@]3(C(=O)c3cc(C(F)(F)F)ccn3)C2)n1 ; O=C(c1ccc(C2CC2)cn1)[C@]12Cc3cnn(-c4ccc(F)cc4)c3C=C1CC[C@H](S(=O)(=O)c1ccccn1)C2 ; O=C(c1ccc(C(F)(F)F)cn1)[C@]12Cc3cnn(-c4ccc(F)cc4)c3C=C1CC[C@H](S(=O)(=O)c1ccccn1)C2] 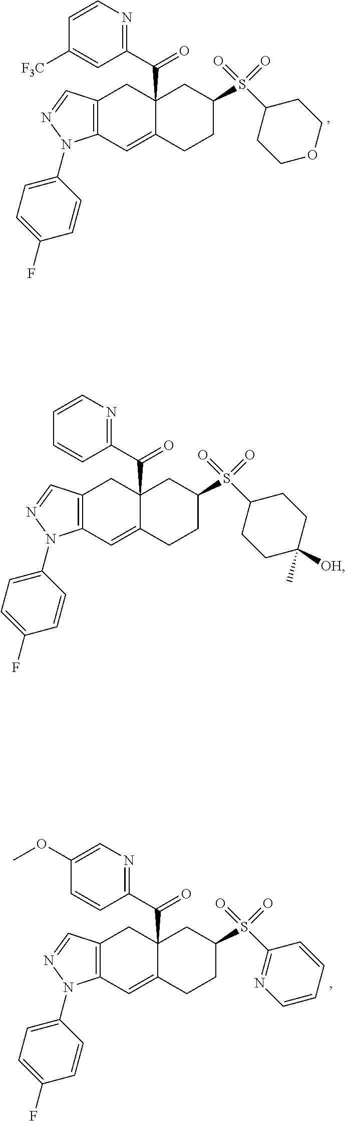 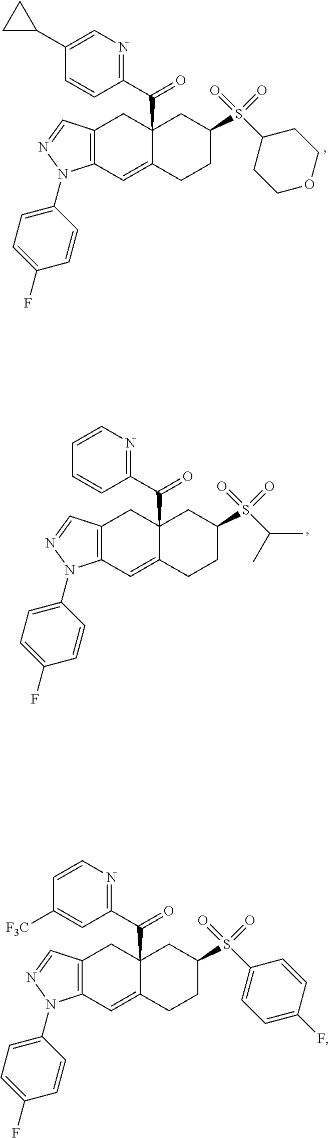 [SMILES: O=C(c1ccc(C2CC2)cn1)[C@]12Cc3cnn(-c4ccc(F)cc4)c3C=C1CC[C@H](S(=O)(=O)C1CCOCC1)C2 ; CC(C)S(=O)(=O)[C@H]1CCC2=Cc3c(cnn3-c3ccc(F)cc3)C[C@]2(C(=O)c2ccccn2)C1 ; O=C(c1cc(C(F)(F)F)ccn1)[C@]12Cc3cnn(-c4ccc(F)cc4)c3C=C1CC[C@H](S(=O)(=O)c1ccc(F)cc1)C2] 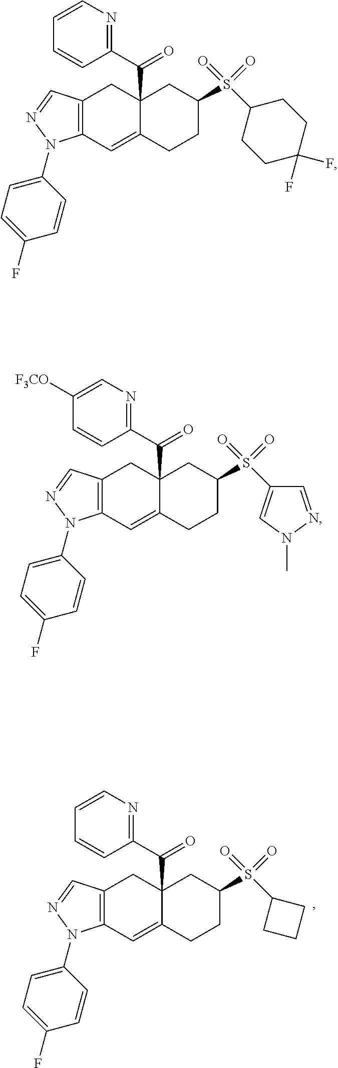 [SMILES: O=C(c1ccccn1)[C@]12Cc3cnn(-c4ccc(F)cc4)c3C=C1CC[C@H](S(=O)(=O)C1CCC(F)(F)CC1)C2 ; Cn1cc(S(=O)(=O)[C@H]2CCC3=Cc4c(cnn4-c4ccc(F)cc4)C[C@]3(C(=O)c3ccc(OC(F)(F)F)cn3)C2)cn1 ; O=C(c1ccccn1)[C@]12Cc3cnn(-c4ccc(F)cc4)c3C=C1CC[C@H](S(=O)(=O)C1CCC1)C2] 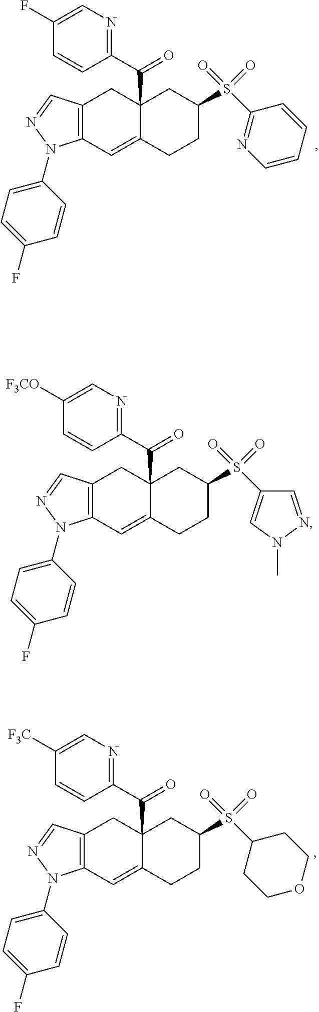 [SMILES: O=C(c1ccc(F)cn1)[C@]12Cc3cnn(-c4ccc(F)cc4)c3C=C1CC[C@H](S(=O)(=O)c1ccccn1)C2 ; Cn1cc(S(=O)(=O)[C@H]2CCC3=Cc4c(cnn4-c4ccc(F)cc4)C[C@]3(C(=O)c3ccc(OC(F)(F)F)cn3)C2)cn1 ; O=C(c1ccc(C(F)(F)F)cn1)[C@]12Cc3cnn(-c4ccc(F)cc4)c3C=C1CC[C@H](S(=O)(=O)C1CCOCC1)C2] 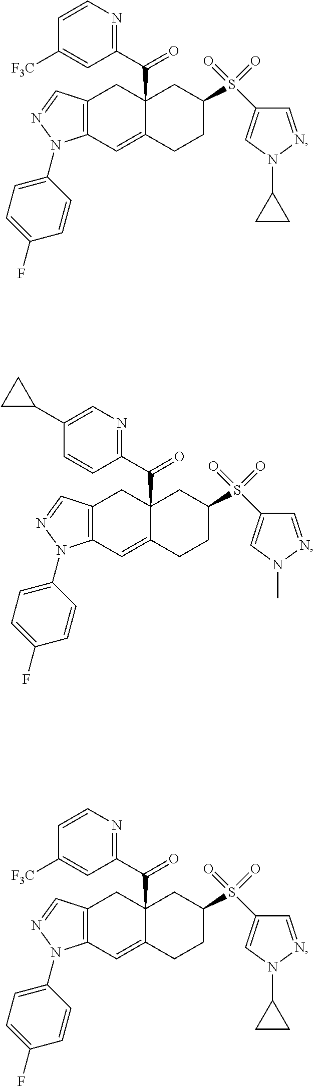 [SMILES: O=C(c1cc(C(F)(F)F)ccn1)[C@]12Cc3cnn(-c4ccc(F)cc4)c3C=C1CC[C@H](S(=O)(=O)c1cnn(C3CC3)c1)C2 ; Cn1cc(S(=O)(=O)[C@H]2CCC3=Cc4c(cnn4-c4ccc(F)cc4)C[C@]3(C(=O)c3ccc(C4CC4)cn3)C2)cn1 ; O=C(c1cc(C(F)(F)F)ccn1)[C@]12Cc3cnn(-c4ccc(F)cc4)c3C=C1CC[C@H](S(=O)(=O)c1cnn(C3CC3)c1)C2] 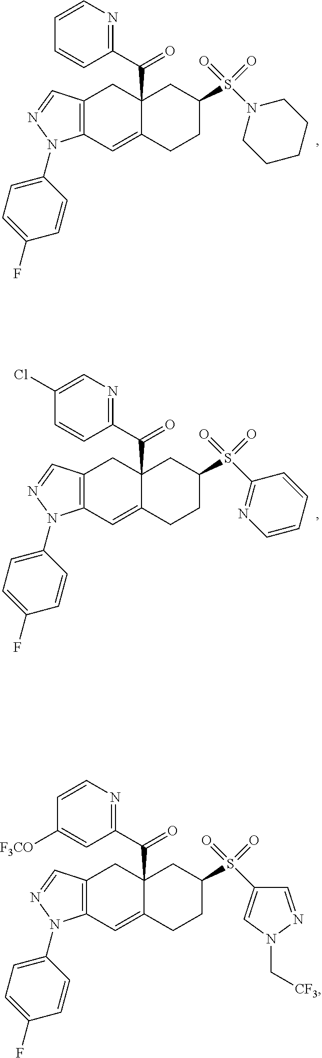 [SMILES: O=C(c1ccccn1)[C@]12Cc3cnn(-c4ccc(F)cc4)c3C=C1CC[C@H](S(=O)(=O)N1CCCCC1)C2 ; O=C(c1ccc(Cl)cn1)[C@]12Cc3cnn(-c4ccc(F)cc4)c3C=C1CC[C@H](S(=O)(=O)c1ccccn1)C2 ; O=C(c1cc(OC(F)(F)F)ccn1)[C@]12Cc3cnn(-c4ccc(F)cc4)c3C=C1CC[C@H](S(=O)(=O)c1cnn(CC(F)(F)F)c1)C2] 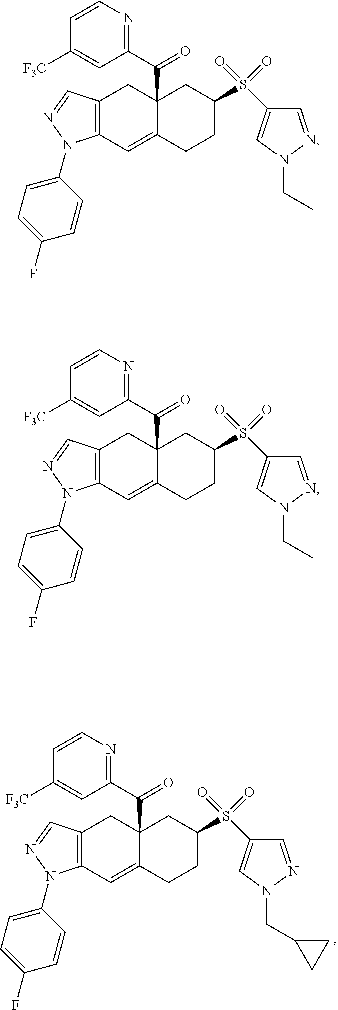 [SMILES: CCn1cc(S(=O)(=O)[C@H]2CCC3=Cc4c(cnn4-c4ccc(F)cc4)C[C@]3(C(=O)c3cc(C(F)(F)F)ccn3)C2)cn1 ; CCn1cc(S(=O)(=O)[C@H]2CCC3=Cc4c(cnn4-c4ccc(F)cc4)C[C@]3(C(=O)c3cc(C(F)(F)F)ccn3)C2)cn1 ; O=C(c1cc(C(F)(F)F)ccn1)[C@]12Cc3cnn(-c4ccc(F)cc4)c3C=C1CC[C@H](S(=O)(=O)c1cnn(CC3CC3)c1)C2] 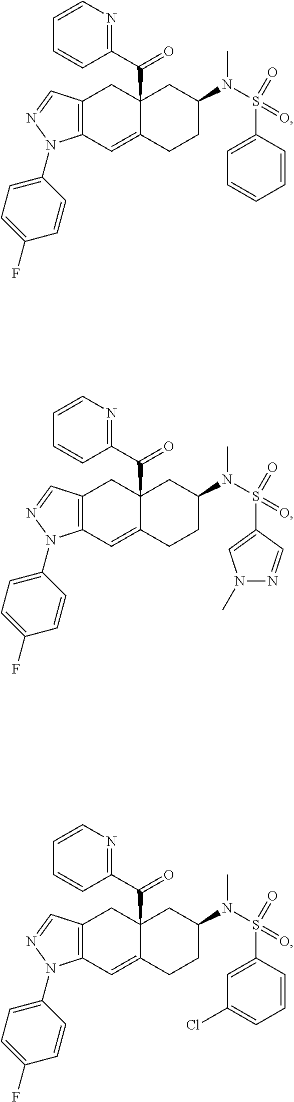 [SMILES: CN([C@H]1CCC2=Cc3c(cnn3-c3ccc(F)cc3)C[C@]2(C(=O)c2ccccn2)C1)S(=O)(=O)c1ccccc1 ; CN([C@H]1CCC2=Cc3c(cnn3-c3ccc(F)cc3)C[C@]2(C(=O)c2ccccn2)C1)S(=O)(=O)c1cnn(C)c1 ; CN([C@H]1CCC2=Cc3c(cnn3-c3ccc(F)cc3)C[C@]2(C(=O)c2ccccn2)C1)S(=O)(=O)c1cccc(Cl)c1] 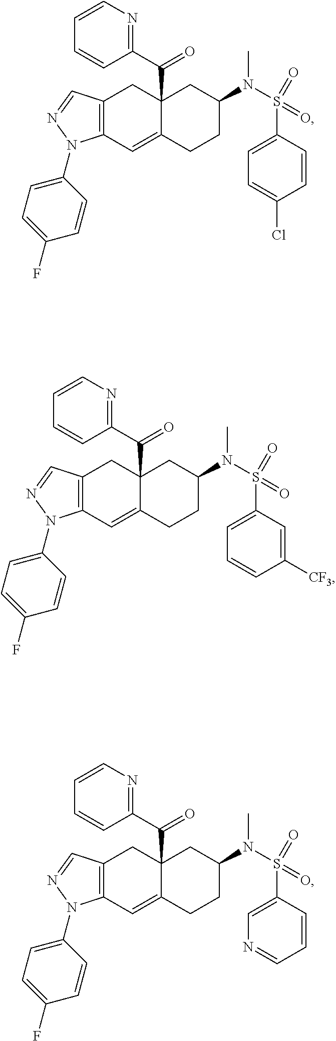 [SMILES: CN([C@H]1CCC2=Cc3c(cnn3-c3ccc(F)cc3)C[C@]2(C(=O)c2ccccn2)C1)S(=O)(=O)c1ccc(Cl)cc1 ; CN([C@H]1CCC2=Cc3c(cnn3-c3ccc(F)cc3)C[C@]2(C(=O)c2ccccn2)C1)S(=O)(=O)c1cccc(C(F)(F)F)c1 ; CN([C@H]1CCC2=Cc3c(cnn3-c3ccc(F)cc3)C[C@]2(C(=O)c2ccccn2)C1)S(=O)(=O)c1cccnc1] 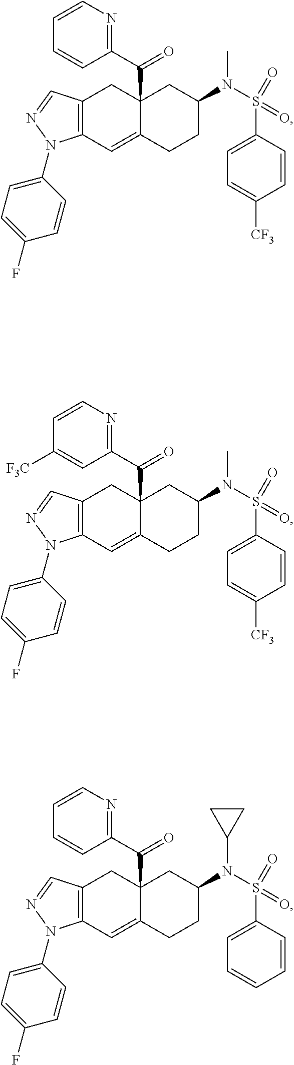 [SMILES: CN([C@H]1CCC2=Cc3c(cnn3-c3ccc(F)cc3)C[C@]2(C(=O)c2ccccn2)C1)S(=O)(=O)c1ccc(C(F)(F)F)cc1 ; CN([C@H]1CCC2=Cc3c(cnn3-c3ccc(F)cc3)C[C@]2(C(=O)c2cc(C(F)(F)F)ccn2)C1)S(=O)(=O)c1ccc(C(F)(F)F)cc1 ; O=C(c1ccccn1)[C@]12Cc3cnn(-c4ccc(F)cc4)c3C=C1CC[C@H](N(C1CC1)S(=O)(=O)c1ccccc1)C2] 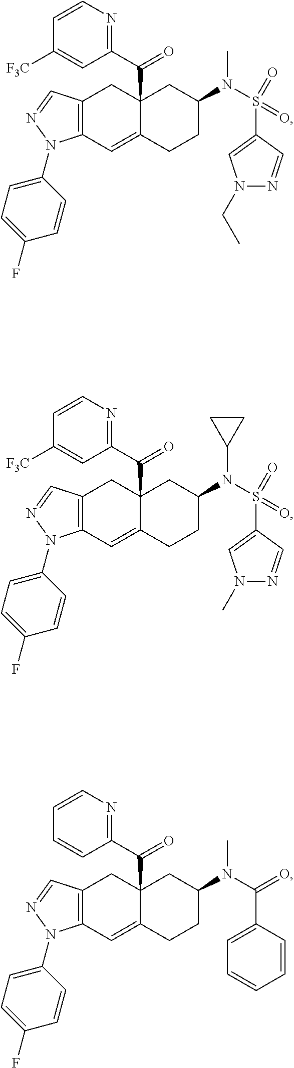 [SMILES: CCn1cc(S(=O)(=O)N(C)[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1 ; CN(C(=O)c1ccccc1)[C@H]1CCC2=Cc3c(cnn3-c3ccc(F)cc3)C[C@]2(C(=O)c2ccccn2)C1] 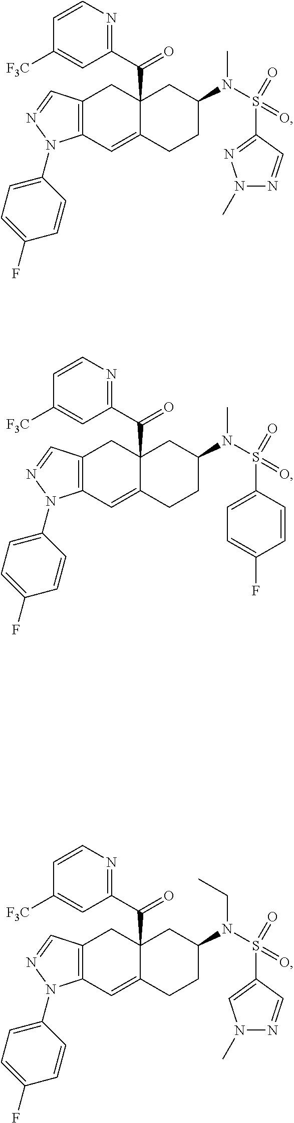 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1ccc(F)cc1 ; CCN([C@H]1CCC2=Cc3c(cnn3-c3ccc(F)cc3)C[C@]2(C(=O)c2cc(C(F)(F)F)ccn2)C1)S(=O)(=O)c1cnn(C)c1] 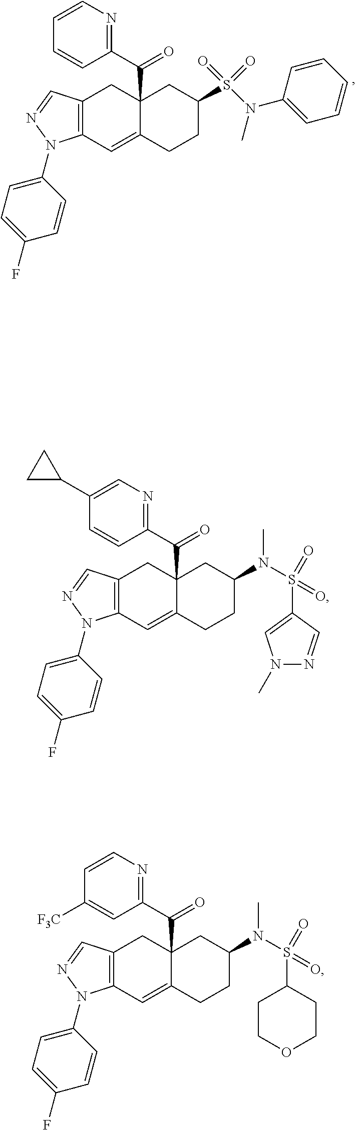 [SMILES: CN(c1ccccc1)S(=O)(=O)[C@H]1CCC2=Cc3c(cnn3-c3ccc(F)cc3)C[C@]2(C(=O)c2ccccn2)C1 ; CN([C@H]1CCC2=Cc3c(cnn3-c3ccc(F)cc3)C[C@]2(C(=O)c2ccc(C3CC3)cn2)C1)S(=O)(=O)c1cnn(C)c1 ; CN([C@H]1CCC2=Cc3c(cnn3-c3ccc(F)cc3)C[C@]2(C(=O)c2cc(C(F)(F)F)ccn2)C1)S(=O)(=O)C1CCOCC1] 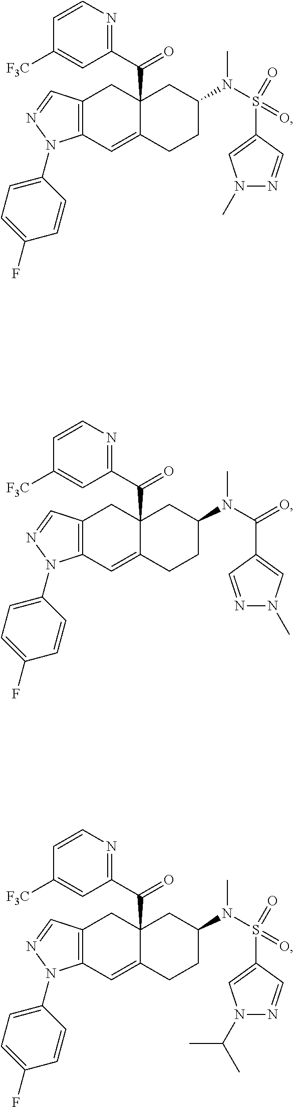 [SMILES: CN([C@@H]1CCC2=Cc3c(cnn3-c3ccc(F)cc3)C[C@]2(C(=O)c2cc(C(F)(F)F)ccn2)C1)S(=O)(=O)c1cnn(C)c1 ; CN(C(=O)c1cnn(C)c1)[C@H]1CCC2=Cc3c(cnn3-c3ccc(F)cc3)C[C@]2(C(=O)c2cc(C(F)(F)F)ccn2)C1 ; CC(C)n1cc(S(=O)(=O)N(C)[C@H]2CCC3=Cc4c(cnn4-c4ccc(F)cc4)C[C@]3(C(=O)c3cc(C(F)(F)F)ccn3)C2)cn1] 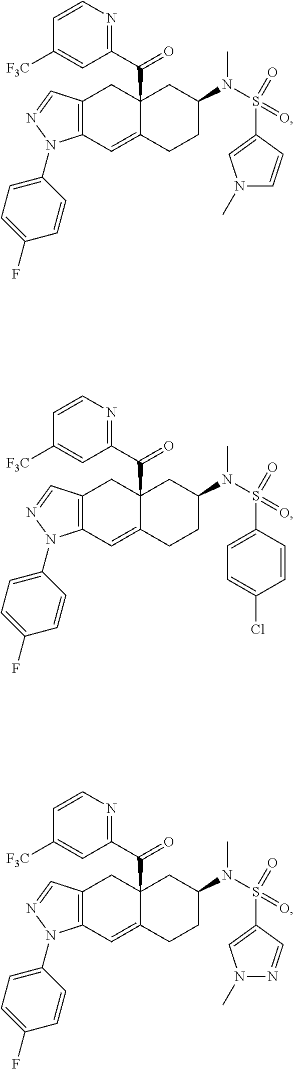 [SMILES: CN([C@H]1CCC2=Cc3c(cnn3-c3ccc(F)cc3)C[C@]2(C(=O)c2cc(C(F)(F)F)ccn2)C1)S(=O)(=O)c1ccn(C)c1 ; CN([C@H]1CCC2=Cc3c(cnn3-c3ccc(F)cc3)C[C@]2(C(=O)c2cc(C(F)(F)F)ccn2)C1)S(=O)(=O)c1ccc(Cl)cc1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)c1] 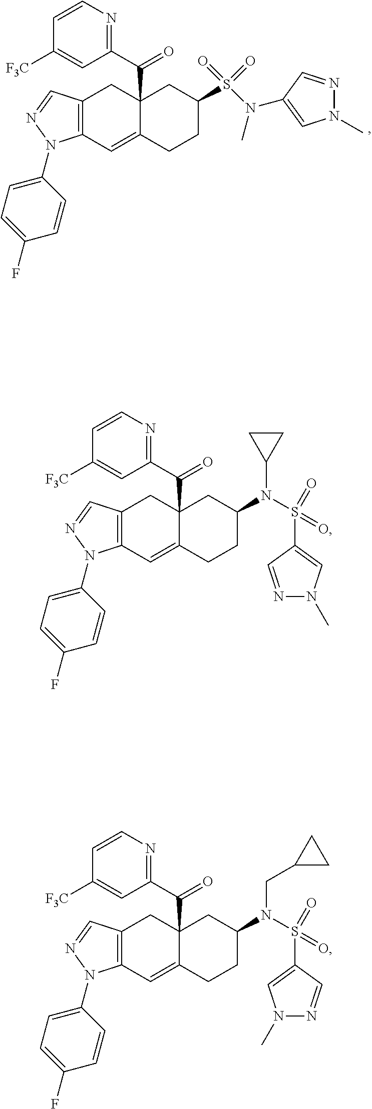 [SMILES: CN(c1cnn(C)c1)S(=O)(=O)[C@H]1CCC2=Cc3c(cnn3-c3ccc(F)cc3)C[C@]2(C(=O)c2cc(C(F)(F)F)ccn2)C1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)cn1] 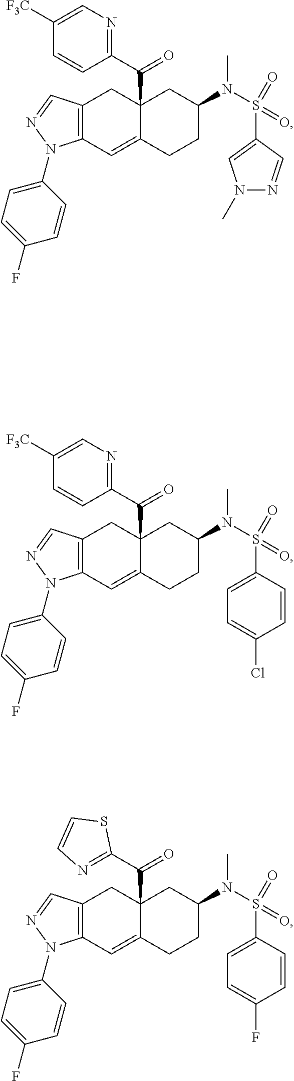 [SMILES: CN([C@H]1CCC2=Cc3c(cnn3-c3ccc(F)cc3)C[C@]2(C(=O)c2ccc(C(F)(F)F)cn2)C1)S(=O)(=O)c1cnn(C)c1 ; CN([C@H]1CCC2=Cc3c(cnn3-c3ccc(F)cc3)C[C@]2(C(=O)c2ccc(C(F)(F)F)cn2)C1)S(=O)(=O)c1ccc(Cl)cc1 ; CN([C@H]1CCC2=Cc3c(cnn3-c3ccc(F)cc3)C[C@]2(C(=O)c2nccs2)C1)S(=O)(=O)c1ccc(F)cc1] 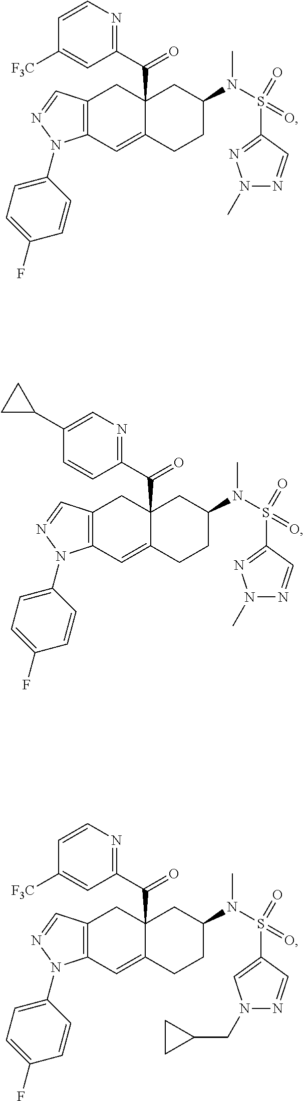 [SMILES: CN([C@H]1CCC2=Cc3c(cnn3-c3ccc(F)cc3)C[C@]2(C(=O)c2cc(C(F)(F)F)ccn2)C1)S(=O)(=O)c1cnn(C)n1 ; CN([C@H]1CCC2=Cc3c(cnn3-c3ccc(F)cc3)C[C@]2(C(=O)c2ccc(C3CC3)cn2)C1)S(=O)(=O)c1cnn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1cnn(CC2CC2)c1] 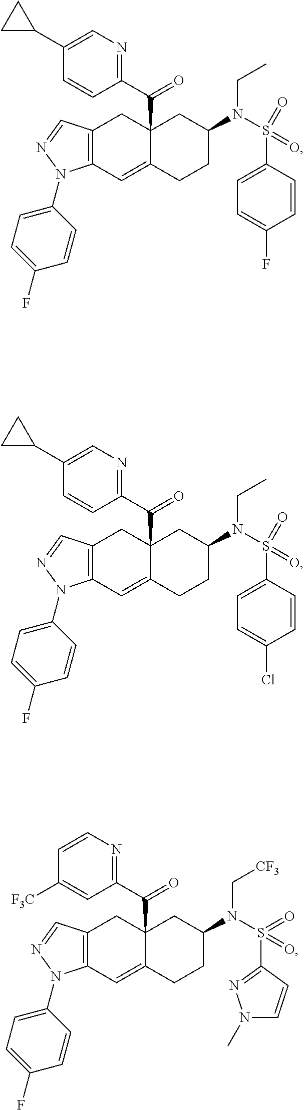 [SMILES: CCN([C@H]1CCC2=Cc3c(cnn3-c3ccc(F)cc3)C[C@]2(C(=O)c2ccc(C3CC3)cn2)C1)S(=O)(=O)c1ccc(F)cc1 ; CCN([C@H]1CCC2=Cc3c(cnn3-c3ccc(F)cc3)C[C@]2(C(=O)c2ccc(C3CC3)cn2)C1)S(=O)(=O)c1ccc(Cl)cc1 ; Cn1ccc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1] 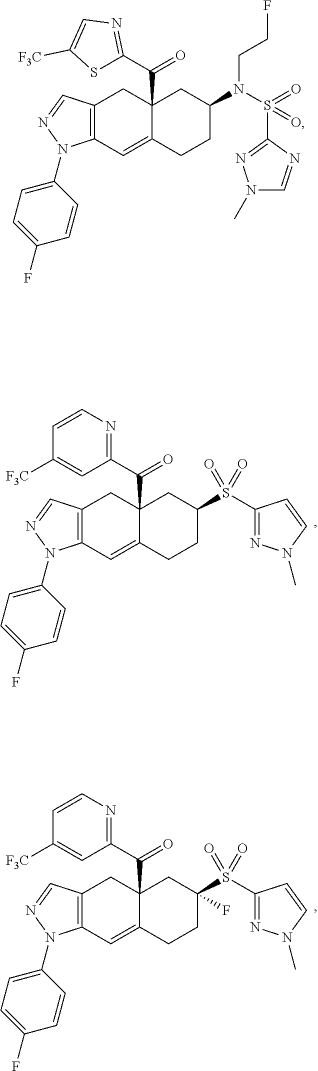 [SMILES: Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)[C@@]2(F)CCC3=Cc4c(cnn4-c4ccc(F)cc4)C[C@]3(C(=O)c3cc(C(F)(F)F)ccn3)C2)n1] 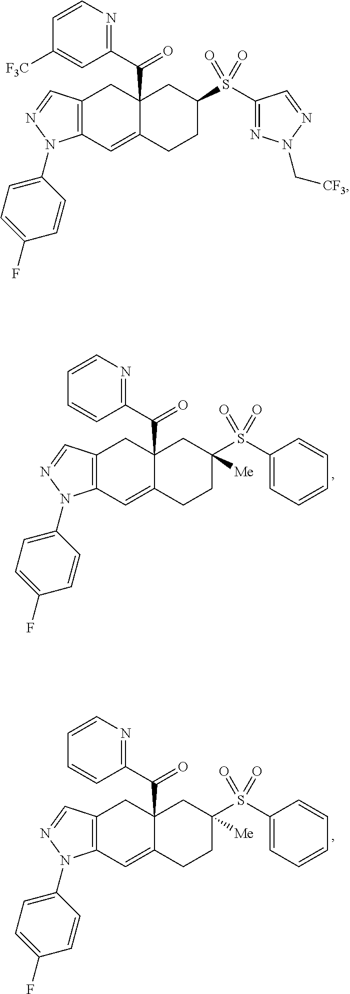 [SMILES: O=C(c1cc(C(F)(F)F)ccn1)[C@]12Cc3cnn(-c4ccc(F)cc4)c3C=C1CC[C@H](S(=O)(=O)c1cnn(CC(F)(F)F)n1)C2 ; C[C@@]1(S(=O)(=O)c2ccccc2)CCC2=Cc3c(cnn3-c3ccc(F)cc3)C[C@]2(C(=O)c2ccccn2)C1 ; C[C@]1(S(=O)(=O)c2ccccc2)CCC2=Cc3c(cnn3-c3ccc(F)cc3)C[C@]2(C(=O)c2ccccn2)C1] 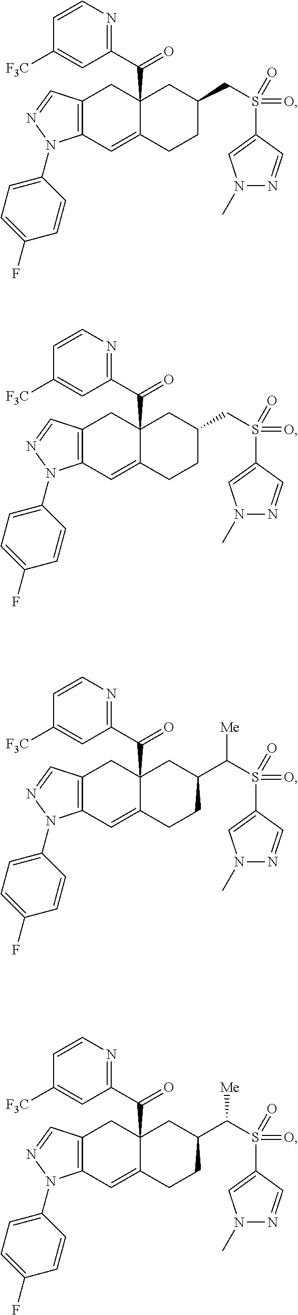 [SMILES: Cn1cc(S(=O)(=O)C[C@H]2CCC3=Cc4c(cnn4-c4ccc(F)cc4)C[C@]3(C(=O)c3cc(C(F)(F)F)ccn3)C2)cn1 ; Cn1cc(S(=O)(=O)C[C@@H]2CCC3=Cc4c(cnn4-c4ccc(F)cc4)C[C@]3(C(=O)c3cc(C(F)(F)F)ccn3)C2)cn1 ; CC([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; C[C@@H]([C@H]1CCC2=Cc3c(cnn3-c3ccc(F)cc3)C[C@]2(C(=O)c2cc(C(F)(F)F)ccn2)C1)S(=O)(=O)c1cnn(C)c1] 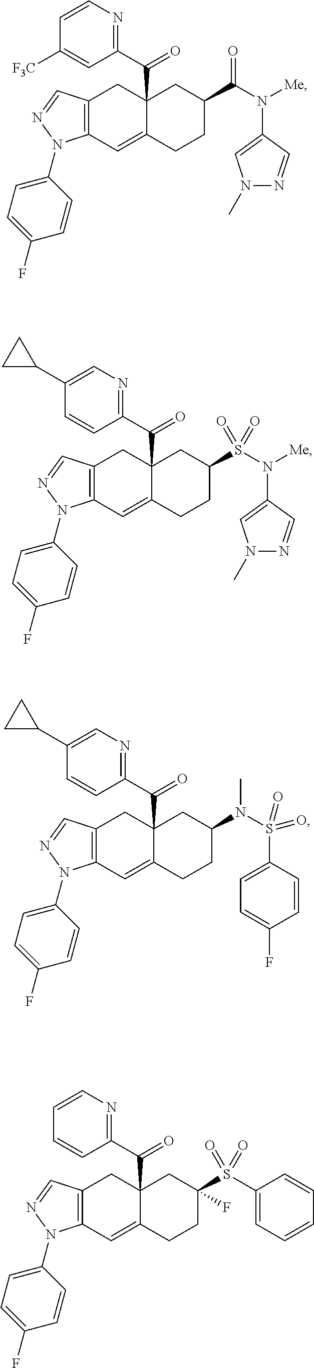 [SMILES: CN(C(=O)[C@H]1CCC2=Cc3c(cnn3-c3ccc(F)cc3)C[C@]2(C(=O)c2cc(C(F)(F)F)ccn2)C1)c1cnn(C)c1 ; CN(c1cnn(C)c1)S(=O)(=O)[C@H]1CCC2=Cc3c(cnn3-c3ccc(F)cc3)C[C@]2(C(=O)c2ccc(C3CC3)cn2)C1 ; CN([C@H]1CCC2=Cc3c(cnn3-c3ccc(F)cc3)C[C@]2(C(=O)c2ccc(C3CC3)cn2)C1)S(=O)(=O)c1ccc(F)cc1 ; O=C(c1ccccn1)[C@]12Cc3cnn(-c4ccc(F)cc4)c3C=C1CC[C@](F)(S(=O)(=O)c1ccccc1)C2] 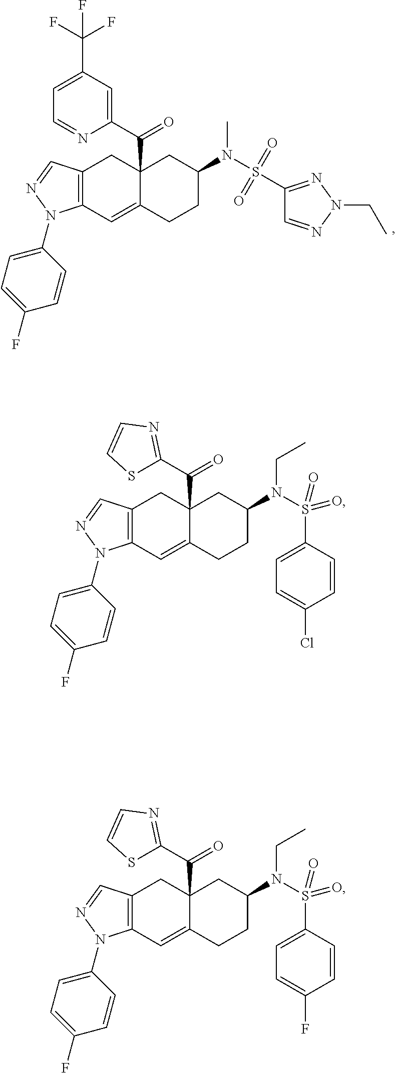 [SMILES: CCn1ncc(S(=O)(=O)N(C)[C@H]2CCC3=Cc4c(cnn4-c4ccc(F)cc4)C[C@]3(C(=O)c3cc(C(F)(F)F)ccn3)C2)n1 ; CCN([C@H]1CCC2=Cc3c(cnn3-c3ccc(F)cc3)C[C@]2(C(=O)c2nccs2)C1)S(=O)(=O)c1ccc(Cl)cc1 ; CCN([C@H]1CCC2=Cc3c(cnn3-c3ccc(F)cc3)C[C@]2(C(=O)c2nccs2)C1)S(=O)(=O)c1ccc(F)cc1] 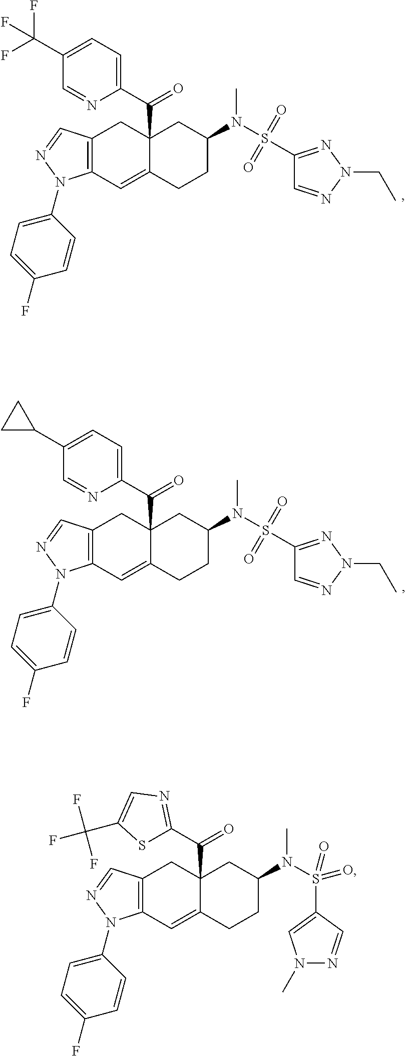 [SMILES: CCn1ncc(S(=O)(=O)N(C)[C@H]2CCC3=Cc4c(cnn4-c4ccc(F)cc4)C[C@]3(C(=O)c3ccc(C(F)(F)F)cn3)C2)n1 ; CCn1ncc(S(=O)(=O)N(C)[C@H]2CCC3=Cc4c(cnn4-c4ccc(F)cc4)C[C@]3(C(=O)c3ccc(C4CC4)cn3)C2)n1 ; CN([C@H]1CCC2=Cc3c(cnn3-c3ccc(F)cc3)C[C@]2(C(=O)c2ncc(C(F)(F)F)s2)C1)S(=O)(=O)c1cnn(C)c1] 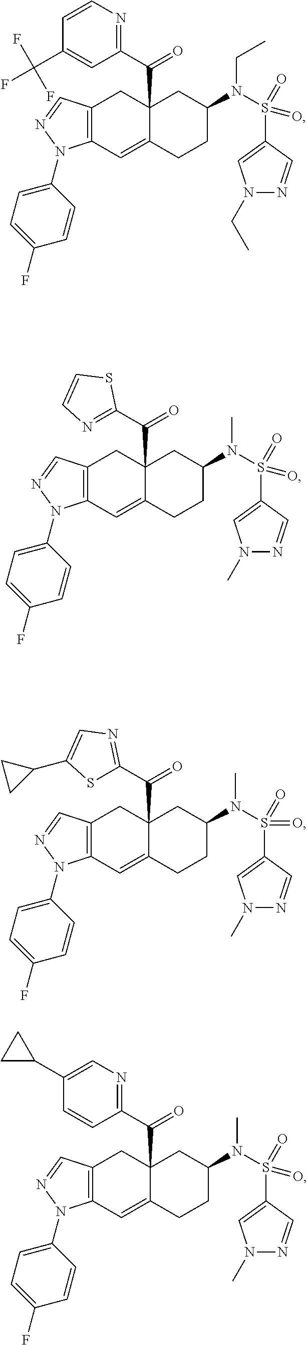 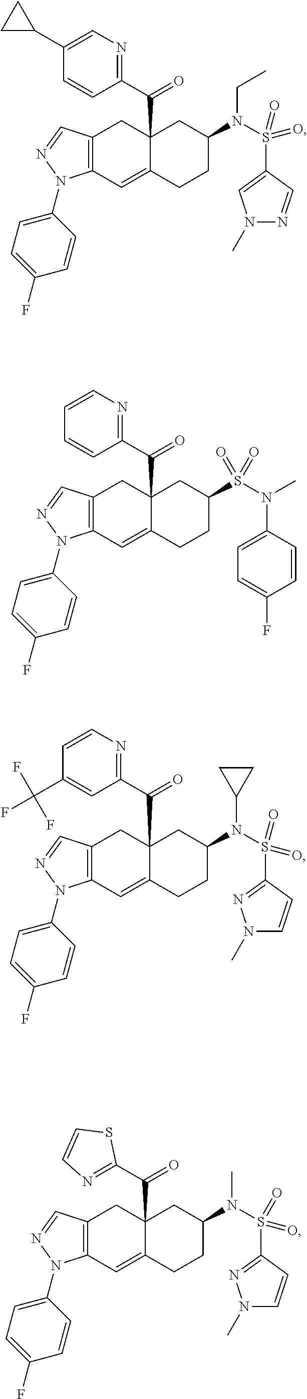 [SMILES: CCN([C@H]1CCC2=Cc3c(cnn3-c3ccc(F)cc3)C[C@]2(C(=O)c2ccc(C3CC3)cn2)C1)S(=O)(=O)c1cnn(C)c1 ; CN(c1ccc(F)cc1)S(=O)(=O)[C@H]1CCC2=Cc3c(cnn3-c3ccc(F)cc3)C[C@]2(C(=O)c2ccccn2)C1 ; Cn1ccc(S(=O)(=O)N(C2CC2)[C@H]2CCC3=Cc4c(cnn4-c4ccc(F)cc4)C[C@]3(C(=O)c3cc(C(F)(F)F)ccn3)C2)n1 ; CN([C@H]1CCC2=Cc3c(cnn3-c3ccc(F)cc3)C[C@]2(C(=O)c2nccs2)C1)S(=O)(=O)c1ccn(C)n1] 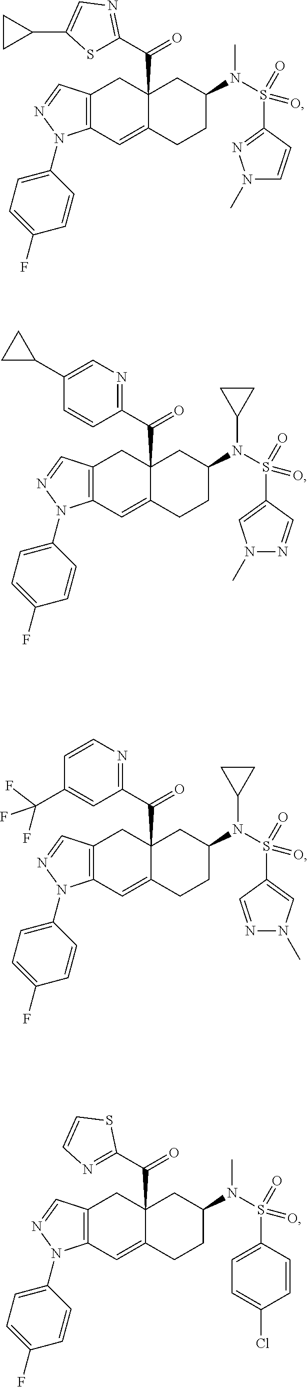 [SMILES: CN([C@H]1CCC2=Cc3c(cnn3-c3ccc(F)cc3)C[C@]2(C(=O)c2ncc(C3CC3)s2)C1)S(=O)(=O)c1ccn(C)n1 ; Cn1cc(S(=O)(=O)N(C2CC2)[C@H]2CCC3=Cc4c(cnn4-c4ccc(F)cc4)C[C@]3(C(=O)c3ccc(C4CC4)cn3)C2)cn1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1 ; CN([C@H]1CCC2=Cc3c(cnn3-c3ccc(F)cc3)C[C@]2(C(=O)c2nccs2)C1)S(=O)(=O)c1ccc(Cl)cc1] 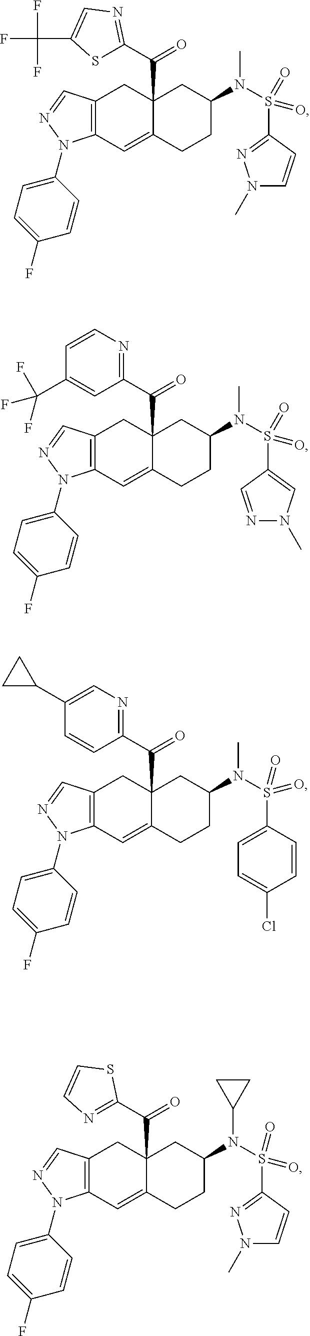 [SMILES: CN([C@H]1CCC2=Cc3c(cnn3-c3ccc(F)cc3)C[C@]2(C(=O)c2ncc(C(F)(F)F)s2)C1)S(=O)(=O)c1ccn(C)n1 ; CN([C@H]1CCC2=Cc3c(cnn3-c3ccc(F)cc3)C[C@]2(C(=O)c2cc(C(F)(F)F)ccn2)C1)S(=O)(=O)c1cnn(C)c1 ; CN([C@H]1CCC2=Cc3c(cnn3-c3ccc(F)cc3)C[C@]2(C(=O)c2ccc(C3CC3)cn2)C1)S(=O)(=O)c1ccc(Cl)cc1 ; Cn1ccc(S(=O)(=O)N(C2CC2)[C@H]2CCC3=Cc4c(cnn4-c4ccc(F)cc4)C[C@]3(C(=O)c3nccs3)C2)n1] 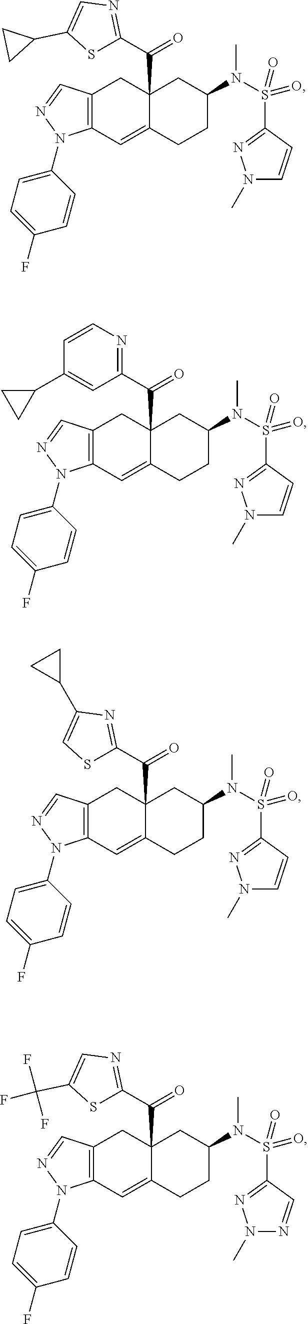 [SMILES: CN([C@H]1CCC2=Cc3c(cnn3-c3ccc(F)cc3)C[C@]2(C(=O)c2ncc(C3CC3)s2)C1)S(=O)(=O)c1ccn(C)n1 ; CN([C@H]1CCC2=Cc3c(cnn3-c3ccc(F)cc3)C[C@]2(C(=O)c2cc(C3CC3)ccn2)C1)S(=O)(=O)c1ccn(C)n1 ; CN([C@H]1CCC2=Cc3c(cnn3-c3ccc(F)cc3)C[C@]2(C(=O)c2nc(C3CC3)cs2)C1)S(=O)(=O)c1ccn(C)n1 ; CN([C@H]1CCC2=Cc3c(cnn3-c3ccc(F)cc3)C[C@]2(C(=O)c2ncc(C(F)(F)F)s2)C1)S(=O)(=O)c1cnn(C)n1] 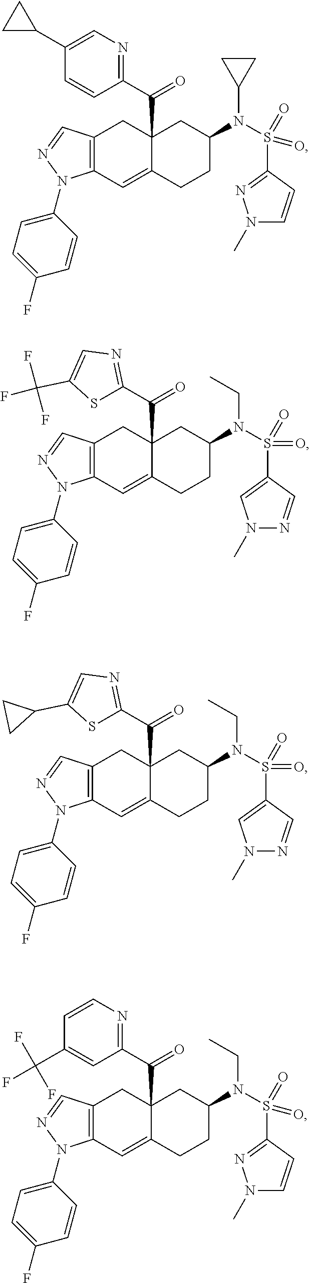 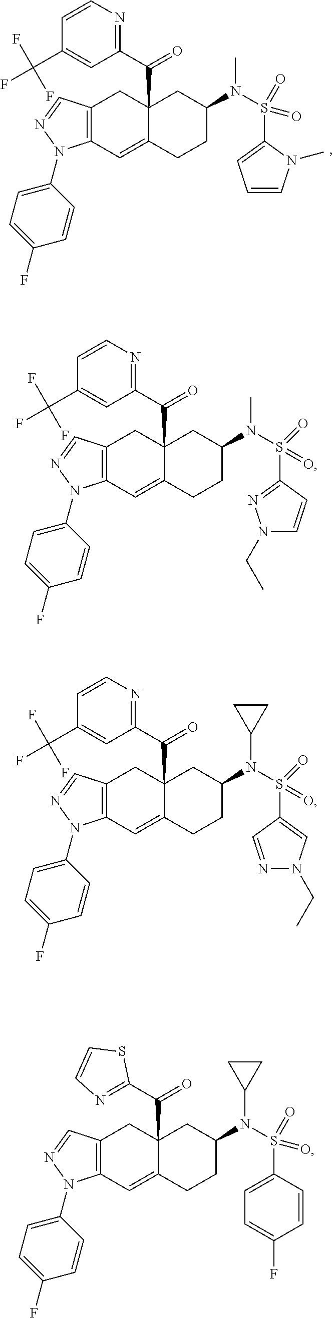 [SMILES: CN([C@H]1CCC2=Cc3c(cnn3-c3ccc(F)cc3)C[C@]2(C(=O)c2cc(C(F)(F)F)ccn2)C1)S(=O)(=O)c1cccn1C ; CCn1ccc(S(=O)(=O)N(C)[C@H]2CCC3=Cc4c(cnn4-c4ccc(F)cc4)C[C@]3(C(=O)c3cc(C(F)(F)F)ccn3)C2)n1 ; CCn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)cn1 ; O=C(c1nccs1)[C@]12Cc3cnn(-c4ccc(F)cc4)c3C=C1CC[C@H](N(C1CC1)S(=O)(=O)c1ccc(F)cc1)C2] 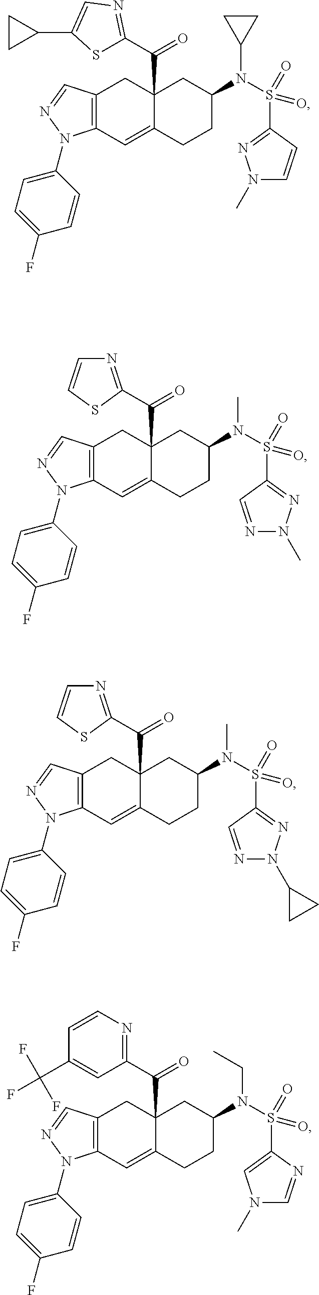 [SMILES: Cn1ccc(S(=O)(=O)N(C2CC2)[C@H]2CCC3=Cc4c(cnn4-c4ccc(F)cc4)C[C@]3(C(=O)c3ncc(C4CC4)s3)C2)n1 ; CN([C@H]1CCC2=Cc3c(cnn3-c3ccc(F)cc3)C[C@]2(C(=O)c2nccs2)C1)S(=O)(=O)c1cnn(C)n1 ; CN([C@H]1CCC2=Cc3c(cnn3-c3ccc(F)cc3)C[C@]2(C(=O)c2nccs2)C1)S(=O)(=O)c1cnn(C2CC2)n1 ; CCN([C@H]1CCC2=Cc3c(cnn3-c3ccc(F)cc3)C[C@]2(C(=O)c2cc(C(F)(F)F)ccn2)C1)S(=O)(=O)c1cn(C)cn1] 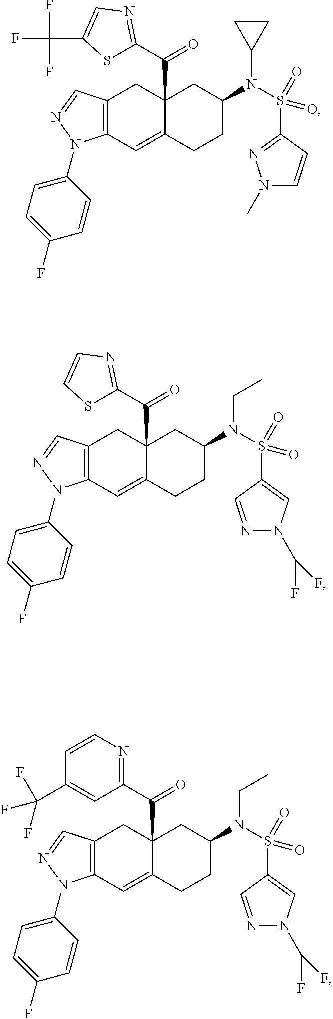 [SMILES: Cn1ccc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; CCN([C@H]1CCC2=Cc3c(cnn3-c3ccc(F)cc3)C[C@]2(C(=O)c2nccs2)C1)S(=O)(=O)c1cnn(C(F)F)c1 ; CCN([C@H]1CCC2=Cc3c(cnn3-c3ccc(F)cc3)C[C@]2(C(=O)c2cc(C(F)(F)F)ccn2)C1)S(=O)(=O)c1cnn(C(F)F)c1] 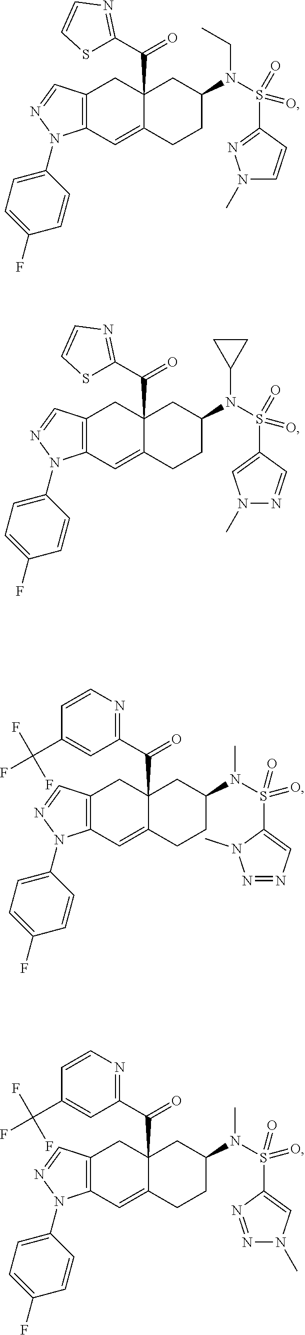 [SMILES: CCN([C@H]1CCC2=Cc3c(cnn3-c3ccc(F)cc3)C[C@]2(C(=O)c2nccs2)C1)S(=O)(=O)c1ccn(C)n1 ; Cn1cc(S(=O)(=O)N(C2CC2)[C@H]2CCC3=Cc4c(cnn4-c4ccc(F)cc4)C[C@]3(C(=O)c3nccs3)C2)cn1 ; CN([C@H]1CCC2=Cc3c(cnn3-c3ccc(F)cc3)C[C@]2(C(=O)c2cc(C(F)(F)F)ccn2)C1)S(=O)(=O)c1cnnn1C ; CN([C@H]1CCC2=Cc3c(cnn3-c3ccc(F)cc3)C[C@]2(C(=O)c2cc(C(F)(F)F)ccn2)C1)S(=O)(=O)c1cn(C)nn1] 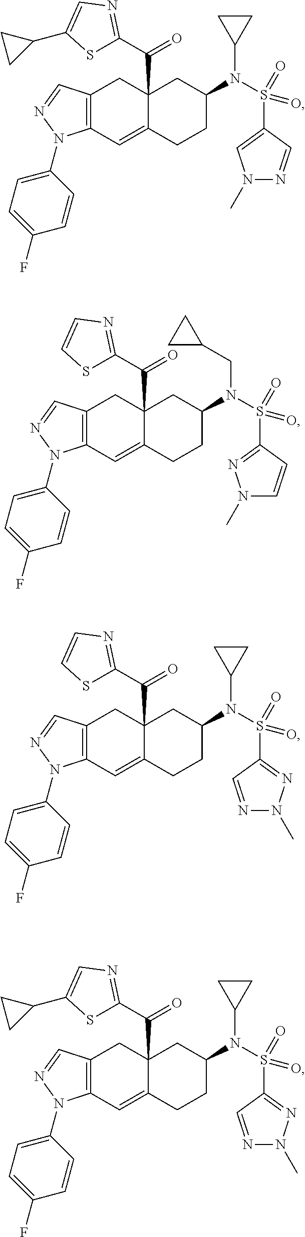 [SMILES: Cn1cc(S(=O)(=O)N(C2CC2)[C@H]2CCC3=Cc4c(cnn4-c4ccc(F)cc4)C[C@]3(C(=O)c3ncc(C4CC4)s3)C2)cn1 ; Cn1ccc(S(=O)(=O)N(CC2CC2)[C@H]2CCC3=Cc4c(cnn4-c4ccc(F)cc4)C[C@]3(C(=O)c3nccs3)C2)n1 ; Cn1ncc(S(=O)(=O)N(C2CC2)[C@H]2CCC3=Cc4c(cnn4-c4ccc(F)cc4)C[C@]3(C(=O)c3nccs3)C2)n1 ; Cn1ncc(S(=O)(=O)N(C2CC2)[C@H]2CCC3=Cc4c(cnn4-c4ccc(F)cc4)C[C@]3(C(=O)c3ncc(C4CC4)s3)C2)n1] 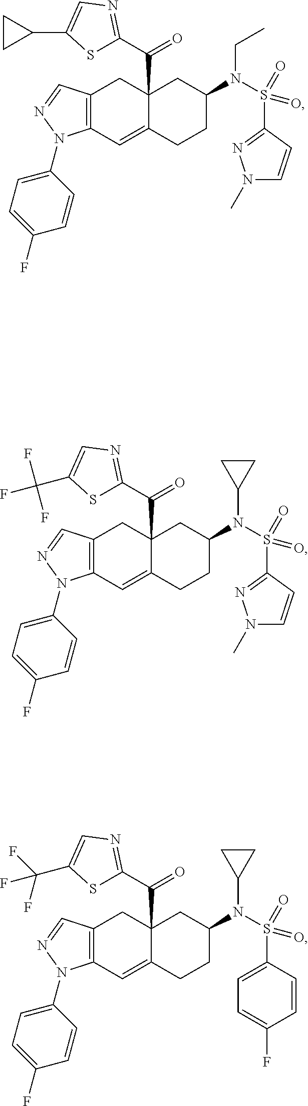 [SMILES: CCN([C@H]1CCC2=Cc3c(cnn3-c3ccc(F)cc3)C[C@]2(C(=O)c2ncc(C3CC3)s2)C1)S(=O)(=O)c1ccn(C)n1 ; Cn1ccc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; O=C(c1ncc(C(F)(F)F)s1)[C@]12Cc3cnn(-c4ccc(F)cc4)c3C=C1CC[C@H](N(C1CC1)S(=O)(=O)c1ccc(F)cc1)C2] 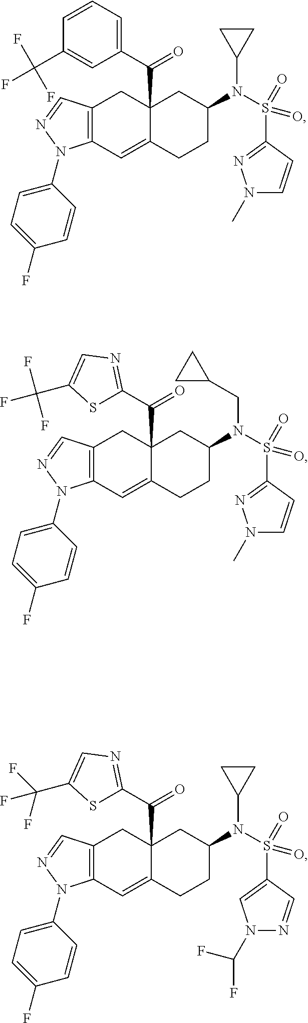 [SMILES: Cn1ccc(S(=O)(=O)N(C2CC2)[C@H]2CCC3=Cc4c(cnn4-c4ccc(F)cc4)C[C@]3(C(=O)c3cccc(C(F)(F)F)c3)C2)n1 ; Cn1ccc(S(=O)(=O)N(CC2CC2)[C@H]2CCC3=Cc4c(cnn4-c4ccc(F)cc4)C[C@]3(C(=O)c3ncc(C(F)(F)F)s3)C2)n1 ; O=C(c1ncc(C(F)(F)F)s1)[C@]12Cc3cnn(-c4ccc(F)cc4)c3C=C1CC[C@H](N(C1CC1)S(=O)(=O)c1cnn(C(F)F)c1)C2] 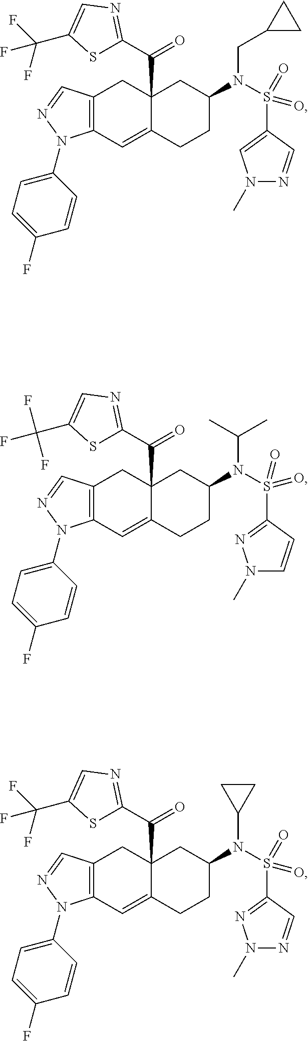 [SMILES: Cn1cc(S(=O)(=O)N(CC2CC2)[C@H]2CCC3=Cc4c(cnn4-c4ccc(F)cc4)C[C@]3(C(=O)c3ncc(C(F)(F)F)s3)C2)cn1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccn(C)n1 ; Cn1ncc(S(=O)(=O)N(C2CC2)[C@H]2CCC3=Cc4c(cnn4-c4ccc(F)cc4)C[C@]3(C(=O)c3ncc(C(F)(F)F)s3)C2)n1] 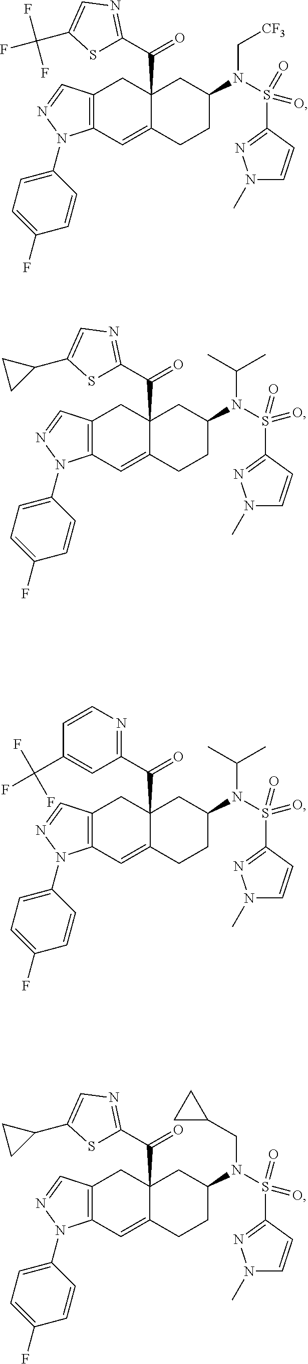 [SMILES: Cn1ccc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1 ; CC(C)N([C@H]1CCC2=Cc3c(cnn3-c3ccc(F)cc3)C[C@]2(C(=O)c2ncc(C3CC3)s2)C1)S(=O)(=O)c1ccn(C)n1 ; CC(C)N([C@H]1CCC2=Cc3c(cnn3-c3ccc(F)cc3)C[C@]2(C(=O)c2cc(C(F)(F)F)ccn2)C1)S(=O)(=O)c1ccn(C)n1 ; Cn1ccc(S(=O)(=O)N(CC2CC2)[C@H]2CCC3=Cc4c(cnn4-c4ccc(F)cc4)C[C@]3(C(=O)c3ncc(C4CC4)s3)C2)n1] 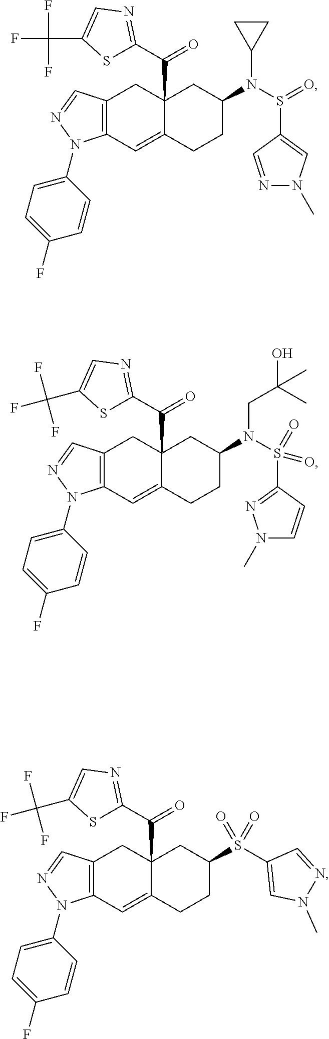 [SMILES: Cn1cc(S(=O)N(C2CC2)[C@H]2CCC3=Cc4c(cnn4-c4ccc(F)cc4)C[C@]3(C(=O)c3ncc(C(F)(F)F)s3)C2)cn1 ; Cn1ccc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1 ; Cn1cc(S(=O)(=O)[C@H]2CCC3=Cc4c(cnn4-c4ccc(F)cc4)C[C@]3(C(=O)c3ncc(C(F)(F)F)s3)C2)cn1] 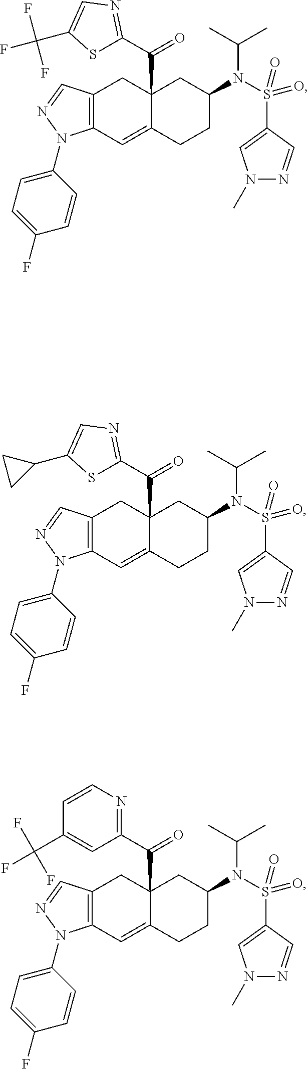 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)c1 ; CC(C)N([C@H]1CCC2=Cc3c(cnn3-c3ccc(F)cc3)C[C@]2(C(=O)c2ncc(C3CC3)s2)C1)S(=O)(=O)c1cnn(C)c1 ; CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)c1] 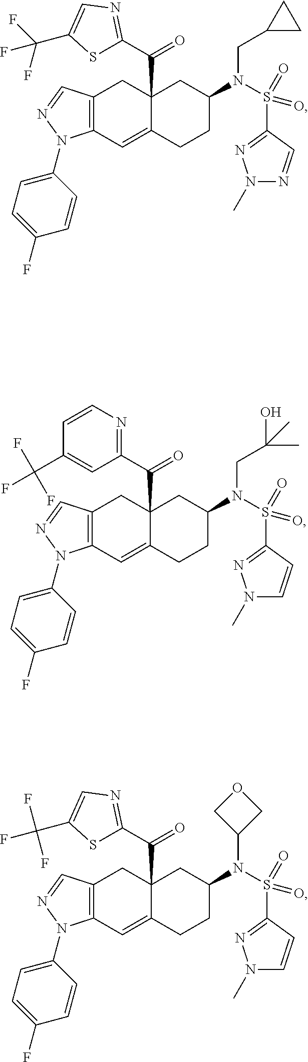 [SMILES: Cn1ncc(S(=O)(=O)N(CC2CC2)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N(CC(C)(C)O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(C2COC2)[C@H]2CCC3=Cc4c(cnn4-c4ccc(F)cc4)C[C@]3(C(=O)c3ncc(C(F)(F)F)s3)C2)n1] 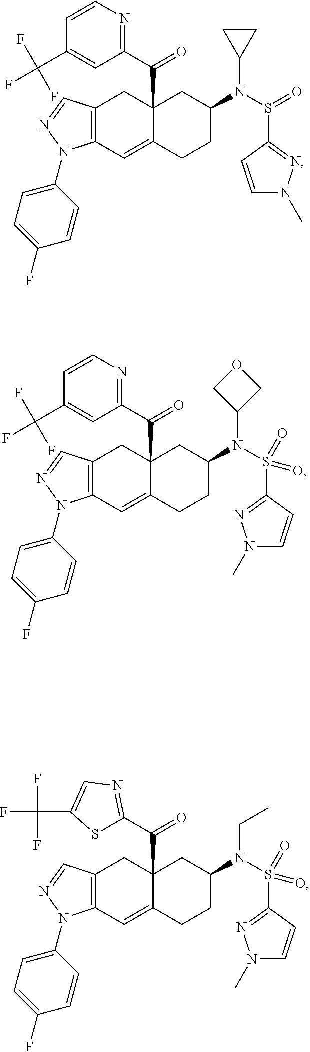 [SMILES: Cn1ccc(S(=O)N(C2CC2)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(C2COC2)[C@H]2CCC3=Cc4c(cnn4-c4ccc(F)cc4)C[C@]3(C(=O)c3cc(C(F)(F)F)ccn3)C2)n1 ; CCN([C@H]1CCC2=Cc3c(cnn3-c3ccc(F)cc3)C[C@]2(C(=O)c2ncc(C(F)(F)F)s2)C1)S(=O)(=O)c1ccn(C)n1] 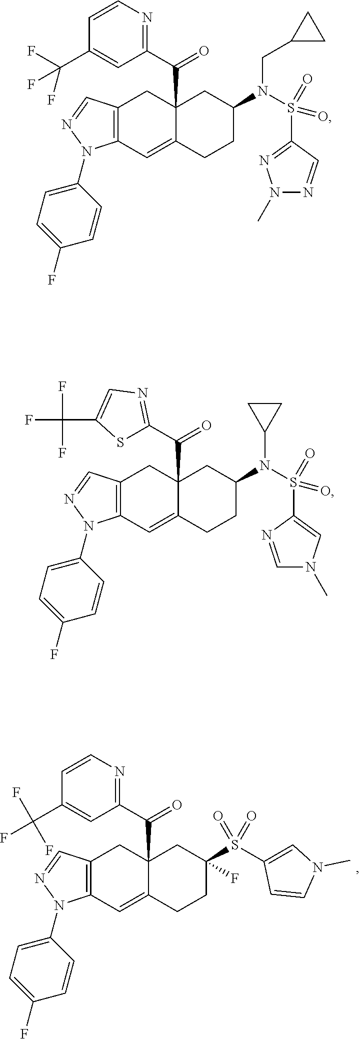 [SMILES: Cn1ncc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)c1 ; Cn1ccc(S(=O)(=O)[C@@]2(F)CCC3=Cc4c(cnn4-c4ccc(F)cc4)C[C@]3(C(=O)c3cc(C(F)(F)F)ccn3)C2)c1] 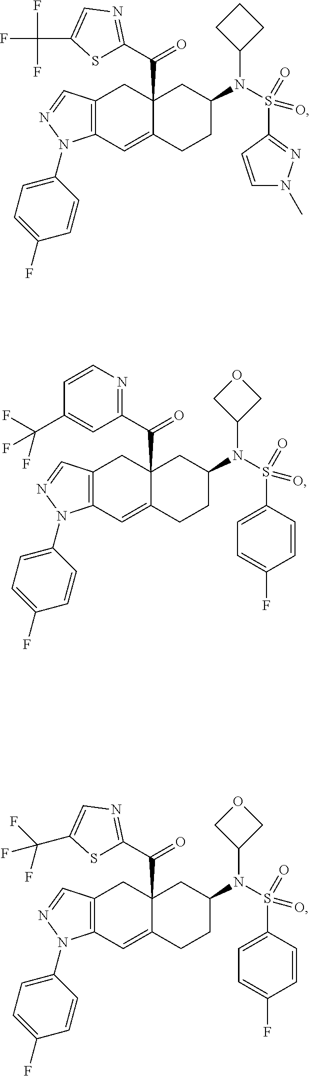 [SMILES: Cn1ccc(S(=O)(=O)N(C2CCC2)[C@H]2CCC3=Cc4c(cnn4-c4ccc(F)cc4)C[C@]3(C(=O)c3ncc(C(F)(F)F)s3)C2)n1 ; O=C(c1cc(C(F)(F)F)ccn1)[C@]12Cc3cnn(-c4ccc(F)cc4)c3C=C1CC[C@H](N(C1COC1)S(=O)(=O)c1ccc(F)cc1)C2 ; O=C(c1ncc(C(F)(F)F)s1)[C@]12Cc3cnn(-c4ccc(F)cc4)c3C=C1CC[C@H](N(C1COC1)S(=O)(=O)c1ccc(F)cc1)C2] 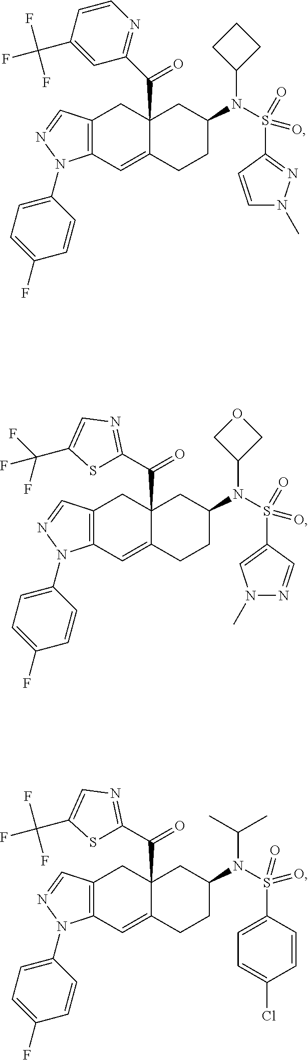 [SMILES: Cn1ccc(S(=O)(=O)N(C2CCC2)[C@H]2CCC3=Cc4c(cnn4-c4ccc(F)cc4)C[C@]3(C(=O)c3cc(C(F)(F)F)ccn3)C2)n1 ; Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)cn1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)cc1] 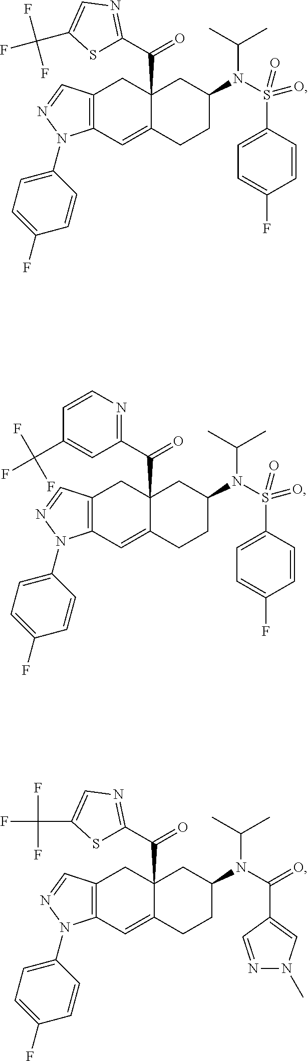 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1ccc(F)cc1 ; CC(C)N([C@H]1CCC2=Cc3c(cnn3-c3ccc(F)cc3)C[C@]2(C(=O)c2cc(C(F)(F)F)ccn2)C1)S(=O)(=O)c1ccc(F)cc1 ; CC(C)N(C(=O)c1cnn(C)c1)[C@H]1CCC2=Cc3c(cnn3-c3ccc(F)cc3)C[C@]2(C(=O)c2ncc(C(F)(F)F)s2)C1] 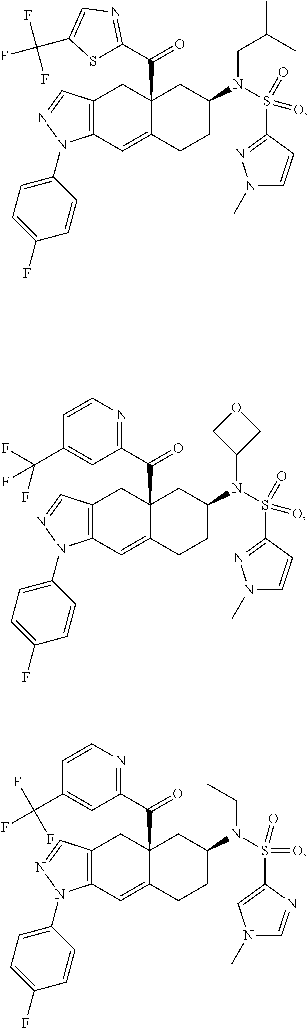 [SMILES: CC(C)CN([C@H]1CCC2=Cc3c(cnn3-c3ccc(F)cc3)C[C@]2(C(=O)c2ncc(C(F)(F)F)s2)C1)S(=O)(=O)c1ccn(C)n1 ; Cn1ccc(S(=O)(=O)N(C2COC2)[C@H]2CCC3=Cc4c(cnn4-c4ccc(F)cc4)C[C@]3(C(=O)c3cc(C(F)(F)F)ccn3)C2)n1 ; CCN([C@H]1CCC2=Cc3c(cnn3-c3ccc(F)cc3)C[C@]2(C(=O)c2cc(C(F)(F)F)ccn2)C1)S(=O)(=O)c1cn(C)cn1] 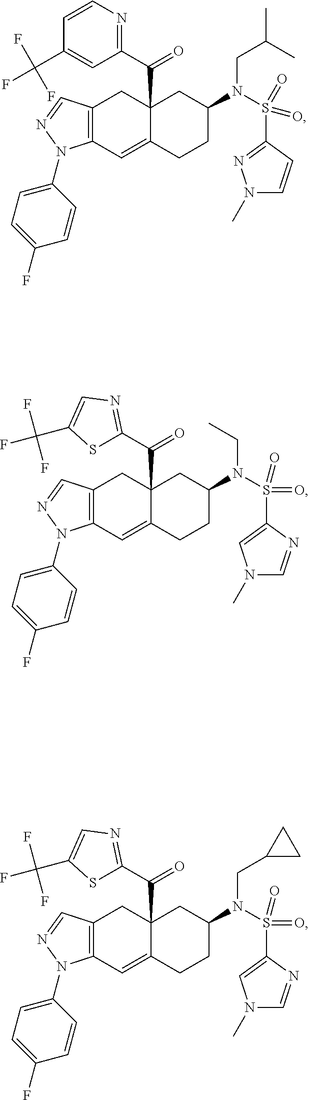 [SMILES: CC(C)CN([C@H]1CCC2=Cc3c(cnn3-c3ccc(F)cc3)C[C@]2(C(=O)c2cc(C(F)(F)F)ccn2)C1)S(=O)(=O)c1ccn(C)n1 ; CCN([C@H]1CCC2=Cc3c(cnn3-c3ccc(F)cc3)C[C@]2(C(=O)c2ncc(C(F)(F)F)s2)C1)S(=O)(=O)c1cn(C)cn1 ; Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)c1] 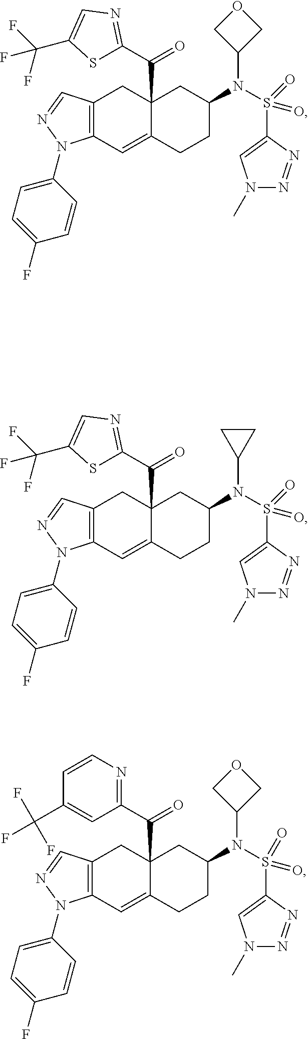 [SMILES: Cn1cc(S(=O)(=O)N(C2COC2)[C@H]2CCC3=Cc4c(cnn4-c4ccc(F)cc4)C[C@]3(C(=O)c3ncc(C(F)(F)F)s3)C2)nn1 ; Cn1cc(S(=O)(=O)N(C2CC2)[C@H]2CCC3=Cc4c(cnn4-c4ccc(F)cc4)C[C@]3(C(=O)c3ncc(C(F)(F)F)s3)C2)nn1 ; Cn1cc(S(=O)(=O)N(C2COC2)[C@H]2CCC3=Cc4c(cnn4-c4ccc(F)cc4)C[C@]3(C(=O)c3cc(C(F)(F)F)ccn3)C2)nn1] 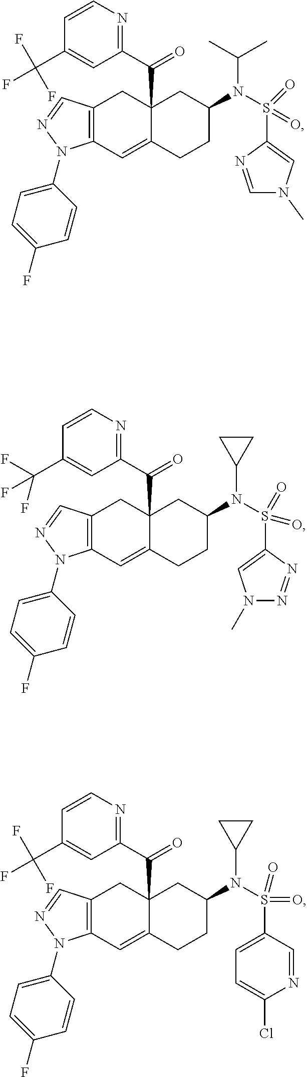 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)cn1 ; Cn1cc(S(=O)(=O)N(C2CC2)[C@H]2CCC3=Cc4c(cnn4-c4ccc(F)cc4)C[C@]3(C(=O)c3cc(C(F)(F)F)ccn3)C2)nn1 ; O=C(c1cc(C(F)(F)F)ccn1)[C@]12Cc3cnn(-c4ccc(F)cc4)c3C=C1CC[C@H](N(C1CC1)S(=O)(=O)c1ccc(Cl)nc1)C2] 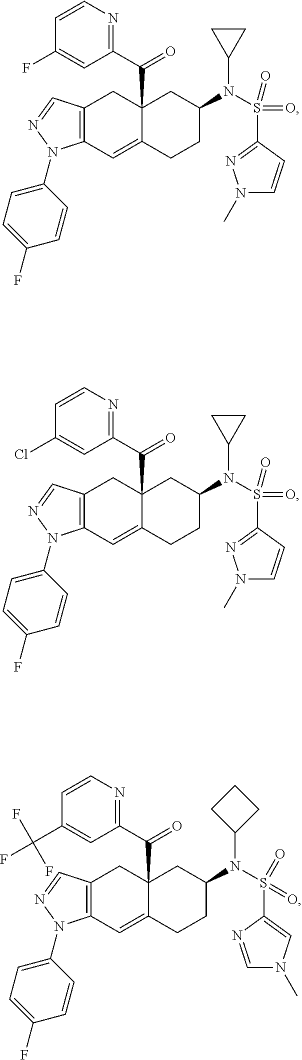 [SMILES: Cn1ccc(S(=O)(=O)N(C2CC2)[C@H]2CCC3=Cc4c(cnn4-c4ccc(F)cc4)C[C@]3(C(=O)c3cc(F)ccn3)C2)n1 ; Cn1ccc(S(=O)(=O)N(C2CC2)[C@H]2CCC3=Cc4c(cnn4-c4ccc(F)cc4)C[C@]3(C(=O)c3cc(Cl)ccn3)C2)n1 ; Cn1cnc(S(=O)(=O)N(C2CCC2)[C@H]2CCC3=Cc4c(cnn4-c4ccc(F)cc4)C[C@]3(C(=O)c3cc(C(F)(F)F)ccn3)C2)c1] 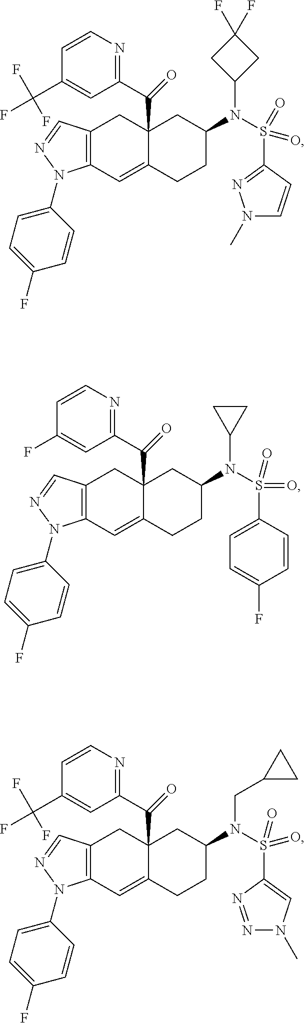 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1 ; O=C(c1cc(F)ccn1)[C@]12Cc3cnn(-c4ccc(F)cc4)c3C=C1CC[C@H](N(C1CC1)S(=O)(=O)c1ccc(F)cc1)C2 ; Cn1cc(S(=O)(=O)N(CC2CC2)[C@H]2CCC3=Cc4c(cnn4-c4ccc(F)cc4)C[C@]3(C(=O)c3cc(C(F)(F)F)ccn3)C2)nn1] 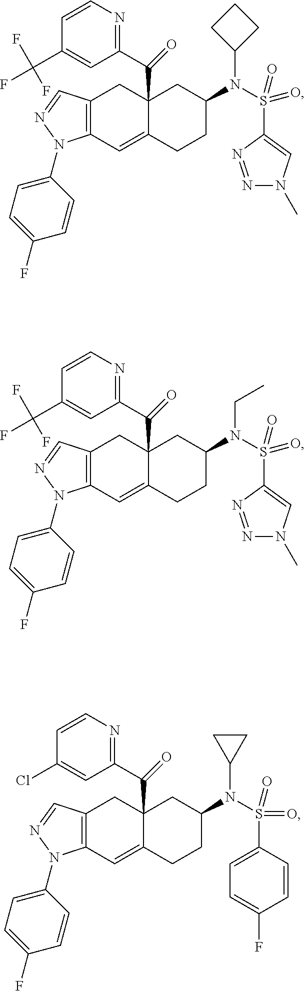 [SMILES: Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)nn1 ; CCN([C@H]1CCC2=Cc3c(cnn3-c3ccc(F)cc3)C[C@]2(C(=O)c2cc(C(F)(F)F)ccn2)C1)S(=O)(=O)c1cn(C)nn1 ; O=C(c1cc(Cl)ccn1)[C@]12Cc3cnn(-c4ccc(F)cc4)c3C=C1CC[C@H](N(C1CC1)S(=O)(=O)c1ccc(F)cc1)C2] 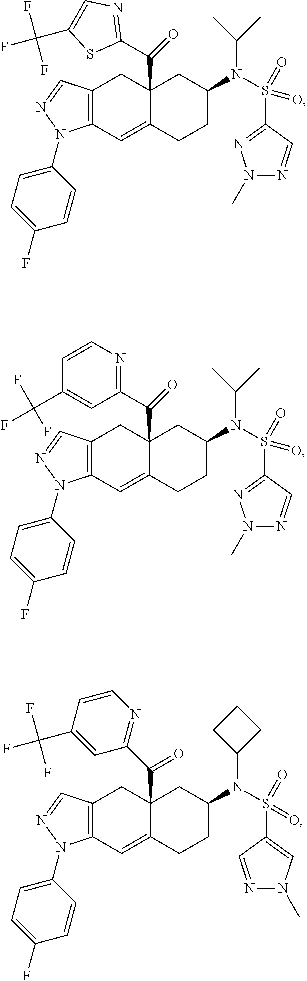 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cnn(C)n1 ; CC(C)N([C@H]1CCC2=Cc3c(cnn3-c3ccc(F)cc3)C[C@]2(C(=O)c2cc(C(F)(F)F)ccn2)C1)S(=O)(=O)c1cnn(C)n1 ; Cn1cc(S(=O)(=O)N(C2CCC2)[C@H]2CCC3=Cc4c(cnn4-c4ccc(F)cc4)C[C@]3(C(=O)c3cc(C(F)(F)F)ccn3)C2)cn1] 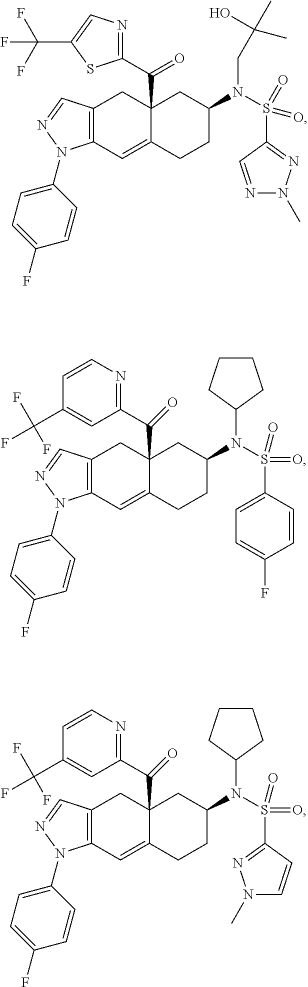 [SMILES: Cn1ncc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)n1 ; O=C(c1cc(C(F)(F)F)ccn1)[C@]12Cc3cnn(-c4ccc(F)cc4)c3C=C1CC[C@H](N(C1CCCC1)S(=O)(=O)c1ccc(F)cc1)C2 ; Cn1ccc(S(=O)(=O)N(C2CCCC2)[C@H]2CCC3=Cc4c(cnn4-c4ccc(F)cc4)C[C@]3(C(=O)c3cc(C(F)(F)F)ccn3)C2)n1] 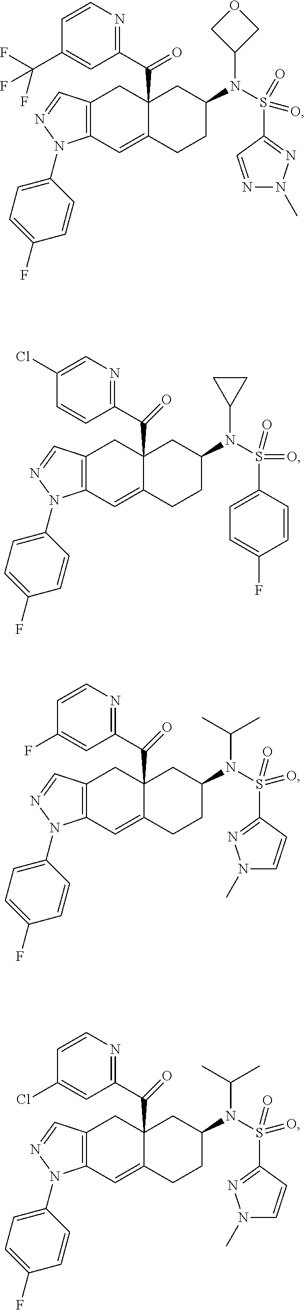 [SMILES: Cn1ncc(S(=O)(=O)N(C2COC2)[C@H]2CCC3=Cc4c(cnn4-c4ccc(F)cc4)C[C@]3(C(=O)c3cc(C(F)(F)F)ccn3)C2)n1 ; O=C(c1ccc(Cl)cn1)[C@]12Cc3cnn(-c4ccc(F)cc4)c3C=C1CC[C@H](N(C1CC1)S(=O)(=O)c1ccc(F)cc1)C2 ; CC(C)N([C@H]1CCC2=Cc3c(cnn3-c3ccc(F)cc3)C[C@]2(C(=O)c2cc(F)ccn2)C1)S(=O)(=O)c1ccn(C)n1 ; CC(C)N([C@H]1CCC2=Cc3c(cnn3-c3ccc(F)cc3)C[C@]2(C(=O)c2cc(Cl)ccn2)C1)S(=O)(=O)c1ccn(C)n1] 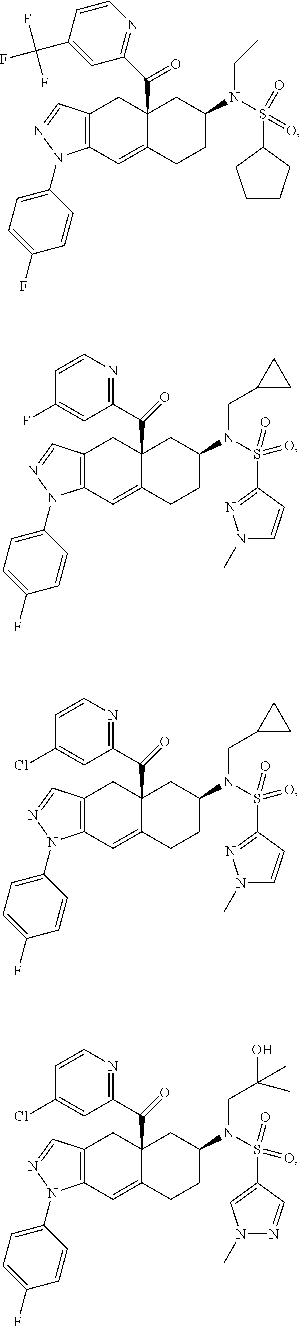 [SMILES: CCN([C@H]1CCC2=Cc3c(cnn3-c3ccc(F)cc3)C[C@]2(C(=O)c2cc(C(F)(F)F)ccn2)C1)S(=O)(=O)C1CCCC1 ; Cn1ccc(S(=O)(=O)N(CC2CC2)[C@H]2CCC3=Cc4c(cnn4-c4ccc(F)cc4)C[C@]3(C(=O)c3cc(F)ccn3)C2)n1 ; Cn1ccc(S(=O)(=O)N(CC2CC2)[C@H]2CCC3=Cc4c(cnn4-c4ccc(F)cc4)C[C@]3(C(=O)c3cc(Cl)ccn3)C2)n1 ; Cn1cc(S(=O)(=O)N(CC(C)(C)O)[C@H]2CCC3=Cc4c(cnn4-c4ccc(F)cc4)C[C@]3(C(=O)c3cc(Cl)ccn3)C2)cn1] 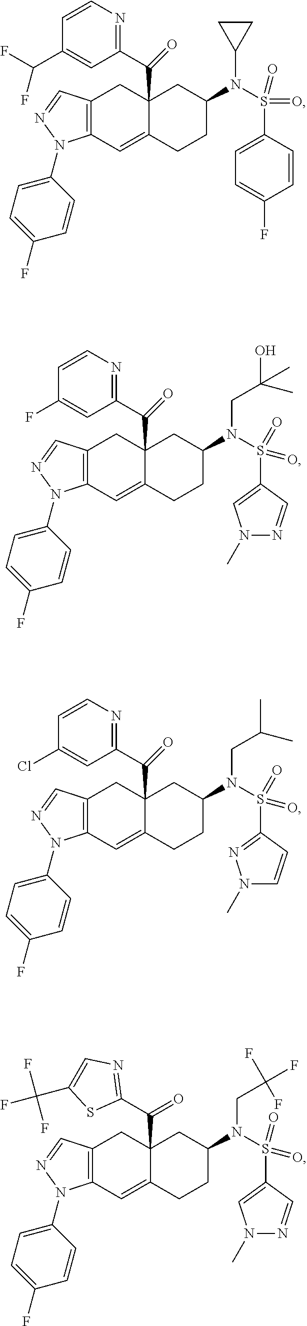 [SMILES: O=C(c1cc(C(F)F)ccn1)[C@]12Cc3cnn(-c4ccc(F)cc4)c3C=C1CC[C@H](N(C1CC1)S(=O)(=O)c1ccc(F)cc1)C2 ; Cn1cc(S(=O)(=O)N(CC(C)(C)O)[C@H]2CCC3=Cc4c(cnn4-c4ccc(F)cc4)C[C@]3(C(=O)c3cc(F)ccn3)C2)cn1 ; CC(C)CN([C@H]1CCC2=Cc3c(cnn3-c3ccc(F)cc3)C[C@]2(C(=O)c2cc(Cl)ccn2)C1)S(=O)(=O)c1ccn(C)n1 ; Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)cn1] 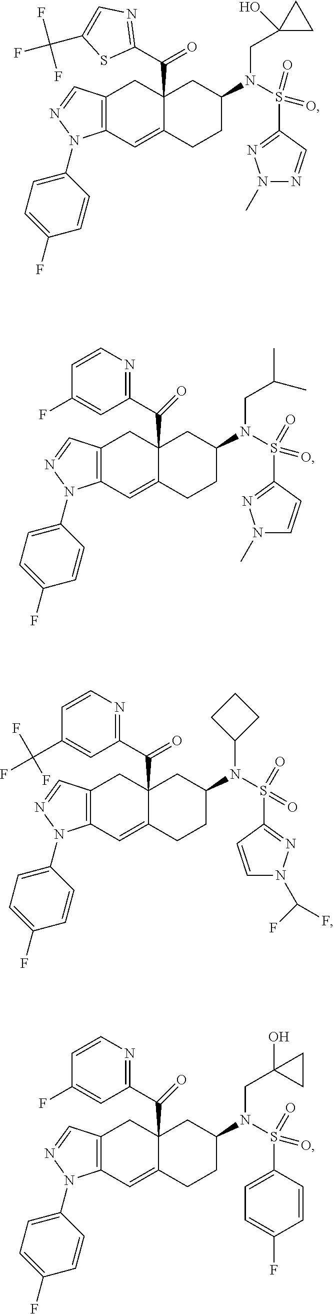 [SMILES: Cn1ncc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1 ; CC(C)CN([C@H]1CCC2=Cc3c(cnn3-c3ccc(F)cc3)C[C@]2(C(=O)c2cc(F)ccn2)C1)S(=O)(=O)c1ccn(C)n1 ; O=C(c1cc(C(F)(F)F)ccn1)[C@]12Cc3cnn(-c4ccc(F)cc4)c3C=C1CC[C@H](N(C1CCC1)S(=O)(=O)c1ccn(C(F)F)n1)C2 ; O=C(c1cc(F)ccn1)[C@]12Cc3cnn(-c4ccc(F)cc4)c3C=C1CC[C@H](N(CC1(O)CC1)S(=O)(=O)c1ccc(F)cc1)C2] 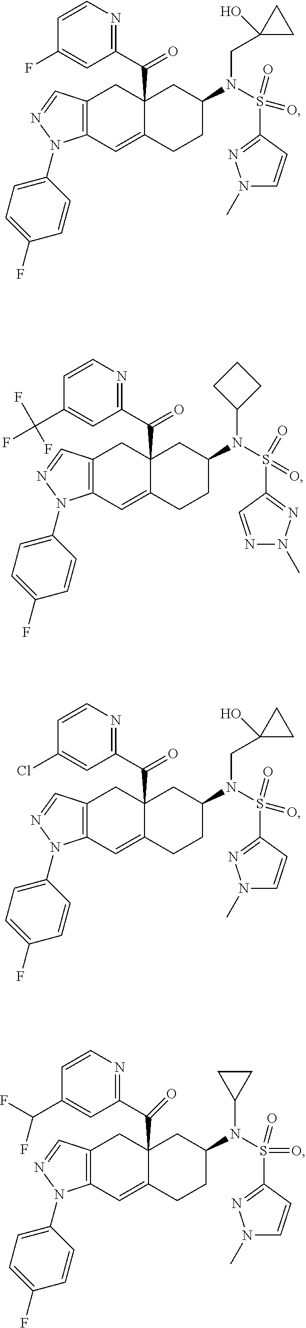 [SMILES: Cn1ccc(S(=O)(=O)N(CC2(O)CC2)[C@H]2CCC3=Cc4c(cnn4-c4ccc(F)cc4)C[C@]3(C(=O)c3cc(F)ccn3)C2)n1 ; Cn1ncc(S(=O)(=O)N(C2CCC2)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CC2(O)CC2)[C@H]2CCC3=Cc4c(cnn4-c4ccc(F)cc4)C[C@]3(C(=O)c3cc(Cl)ccn3)C2)n1 ; Cn1ccc(S(=O)(=O)N(C2CC2)[C@H]2CCC3=Cc4c(cnn4-c4ccc(F)cc4)C[C@]3(C(=O)c3cc(C(F)F)ccn3)C2)n1] 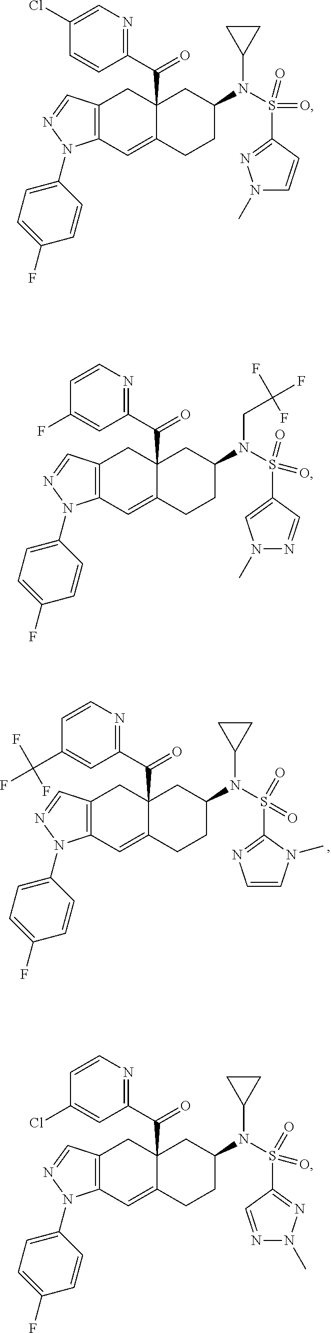 [SMILES: Cn1ccc(S(=O)(=O)N(C2CC2)[C@H]2CCC3=Cc4c(cnn4-c4ccc(F)cc4)C[C@]3(C(=O)c3ccc(Cl)cn3)C2)n1 ; Cn1cc(S(=O)(=O)N(CC(F)(F)F)[C@H]2CCC3=Cc4c(cnn4-c4ccc(F)cc4)C[C@]3(C(=O)c3cc(F)ccn3)C2)cn1 ; Cn1ccnc1S(=O)(=O)N(C1CC1)[C@H]1CCC2=Cc3c(cnn3-c3ccc(F)cc3)C[C@]2(C(=O)c2cc(C(F)(F)F)ccn2)C1 ; Cn1ncc(S(=O)(=O)N(C2CC2)[C@H]2CCC3=Cc4c(cnn4-c4ccc(F)cc4)C[C@]3(C(=O)c3cc(Cl)ccn3)C2)n1] 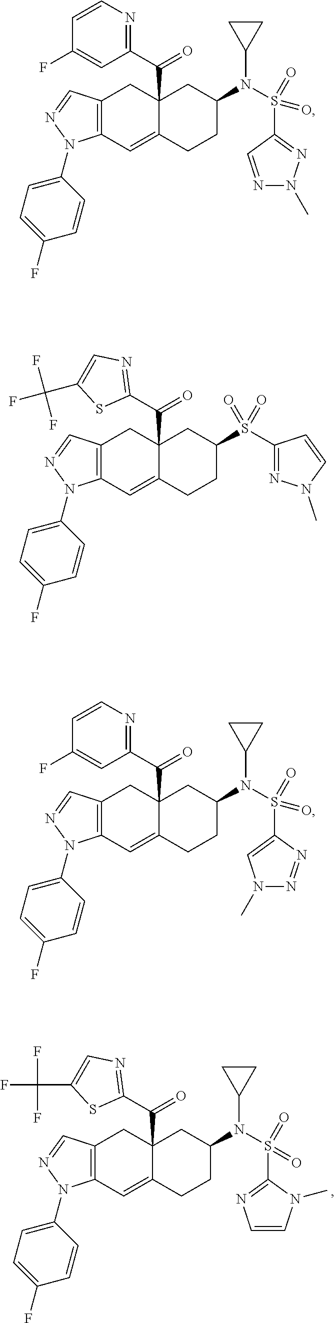 [SMILES: Cn1ncc(S(=O)(=O)N(C2CC2)[C@H]2CCC3=Cc4c(cnn4-c4ccc(F)cc4)C[C@]3(C(=O)c3cc(F)ccn3)C2)n1 ; Cn1ccc(S(=O)(=O)[C@H]2CCC3=Cc4c(cnn4-c4ccc(F)cc4)C[C@]3(C(=O)c3ncc(C(F)(F)F)s3)C2)n1 ; Cn1cc(S(=O)(=O)N(C2CC2)[C@H]2CCC3=Cc4c(cnn4-c4ccc(F)cc4)C[C@]3(C(=O)c3cc(F)ccn3)C2)nn1 ; Cn1ccnc1S(=O)(=O)N(C1CC1)[C@H]1CCC2=Cc3c(cnn3-c3ccc(F)cc3)C[C@]2(C(=O)c2ncc(C(F)(F)F)s2)C1] 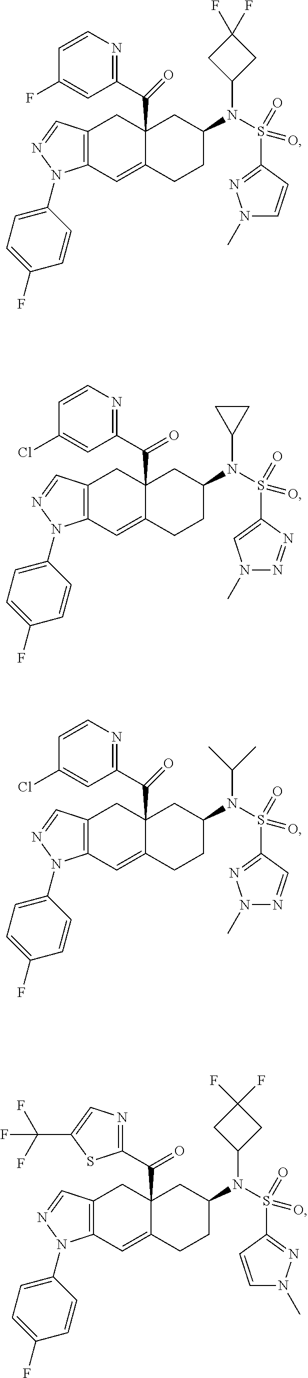 [SMILES: Cn1ccc(S(=O)(=O)N(C2CC(F)(F)C2)[C@H]2CCC3=Cc4c(cnn4-c4ccc(F)cc4)C[C@]3(C(=O)c3cc(F)ccn3)C2)n1 ; Cn1cc(S(=O)(=O)N(C2CC2)[C@H]2CCC3=Cc4c(cnn4-c4ccc(F)cc4)C[C@]3(C(=O)c3cc(Cl)ccn3)C2)nn1 ; CC(C)N([C@H]1CCC2=Cc3c(cnn3-c3ccc(F)cc3)C[C@]2(C(=O)c2cc(Cl)ccn2)C1)S(=O)(=O)c1cnn(C)n1 ; Cn1ccc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1] 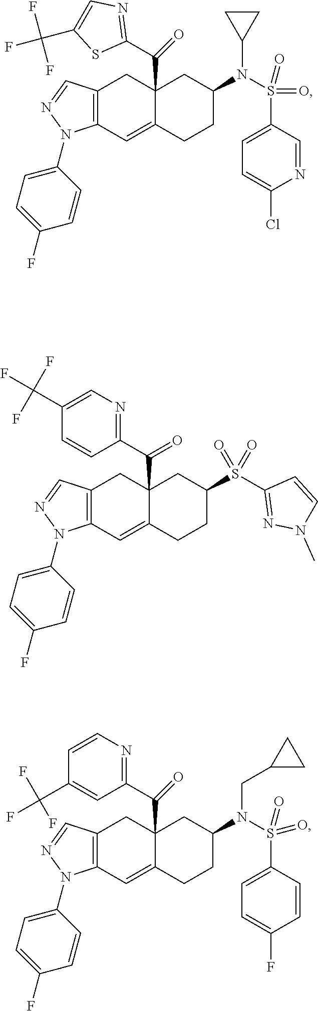 [SMILES: O=C(c1ncc(C(F)(F)F)s1)[C@]12Cc3cnn(-c4ccc(F)cc4)c3C=C1CC[C@H](N(C1CC1)S(=O)(=O)c1ccc(Cl)nc1)C2 ; Cn1ccc(S(=O)(=O)[C@H]2CCC3=Cc4c(cnn4-c4ccc(F)cc4)C[C@]3(C(=O)c3ccc(C(F)(F)F)cn3)C2)n1 ; O=C(c1cc(C(F)(F)F)ccn1)[C@]12Cc3cnn(-c4ccc(F)cc4)c3C=C1CC[C@H](N(CC1CC1)S(=O)(=O)c1ccc(F)cc1)C2] 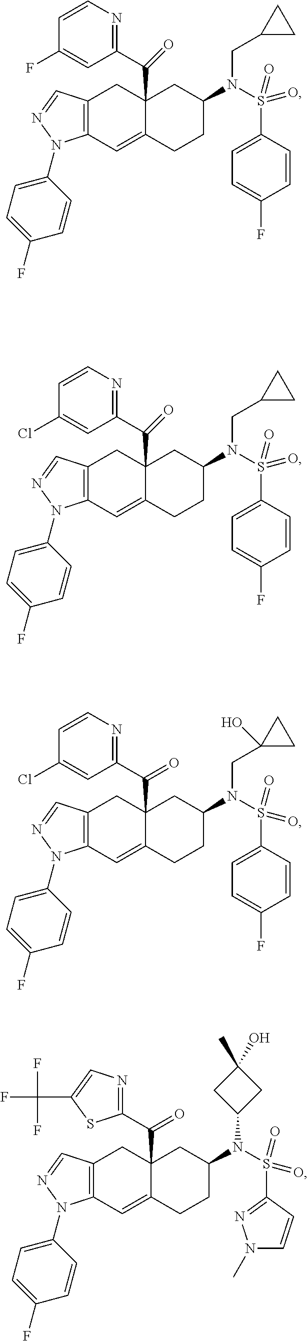 [SMILES: O=C(c1cc(F)ccn1)[C@]12Cc3cnn(-c4ccc(F)cc4)c3C=C1CC[C@H](N(CC1CC1)S(=O)(=O)c1ccc(F)cc1)C2 ; O=C(c1cc(Cl)ccn1)[C@]12Cc3cnn(-c4ccc(F)cc4)c3C=C1CC[C@H](N(CC1CC1)S(=O)(=O)c1ccc(F)cc1)C2 ; O=C(c1cc(Cl)ccn1)[C@]12Cc3cnn(-c4ccc(F)cc4)c3C=C1CC[C@H](N(CC1(O)CC1)S(=O)(=O)c1ccc(F)cc1)C2 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@](C)(O)C2)n1] 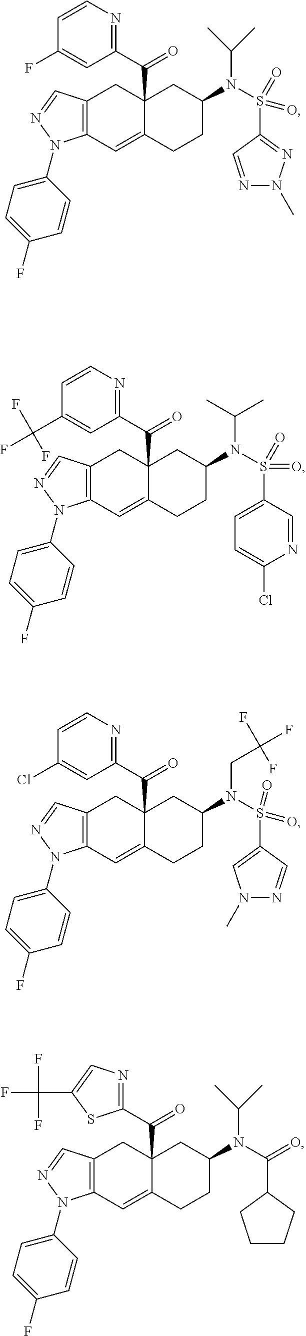 [SMILES: CC(C)N([C@H]1CCC2=Cc3c(cnn3-c3ccc(F)cc3)C[C@]2(C(=O)c2cc(F)ccn2)C1)S(=O)(=O)c1cnn(C)n1 ; CC(C)N([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1 ; Cn1cc(S(=O)(=O)N(CC(F)(F)F)[C@H]2CCC3=Cc4c(cnn4-c4ccc(F)cc4)C[C@]3(C(=O)c3cc(Cl)ccn3)C2)cn1 ; CC(C)N(C(=O)C1CCCC1)[C@H]1CCC2=Cc3c(cnn3-c3ccc(F)cc3)C[C@]2(C(=O)c2ncc(C(F)(F)F)s2)C1] 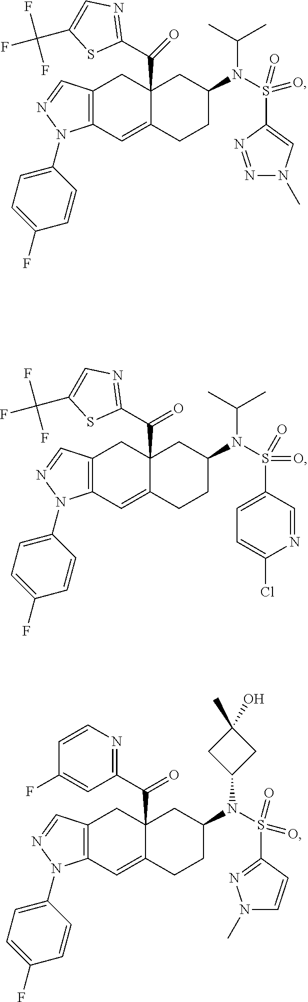 [SMILES: CC(C)N([C@H]1CCC2=Cc3c(cnn3-c3ccc(F)cc3)C[C@]2(C(=O)c2ncc(C(F)(F)F)s2)C1)S(=O)(=O)c1cn(C)nn1 ; CC(C)N([C@H]1CCC2=Cc3c(cnn3-c3ccc(F)cc3)C[C@]2(C(=O)c2ncc(C(F)(F)F)s2)C1)S(=O)(=O)c1ccc(Cl)nc1 ; Cn1ccc(S(=O)(=O)N([C@H]2CCC3=Cc4c(cnn4-c4ccc(F)cc4)C[C@]3(C(=O)c3cc(F)ccn3)C2)[C@H]2C[C@@](C)(O)C2)n1] 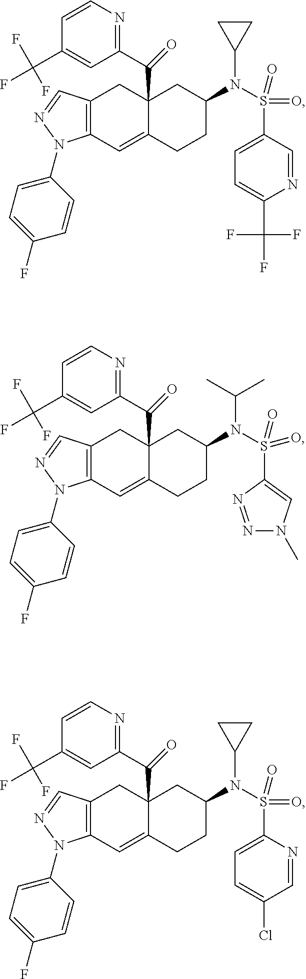 [SMILES: O=C(c1cc(C(F)(F)F)ccn1)[C@]12Cc3cnn(-c4ccc(F)cc4)c3C=C1CC[C@H](N(C1CC1)S(=O)(=O)c1ccc(C(F)(F)F)nc1)C2 ; CC(C)N([C@H]1CCC2=Cc3c(cnn3-c3ccc(F)cc3)C[C@]2(C(=O)c2cc(C(F)(F)F)ccn2)C1)S(=O)(=O)c1cn(C)nn1 ; O=C(c1cc(C(F)(F)F)ccn1)[C@]12Cc3cnn(-c4ccc(F)cc4)c3C=C1CC[C@H](N(C1CC1)S(=O)(=O)c1ccc(Cl)cn1)C2] 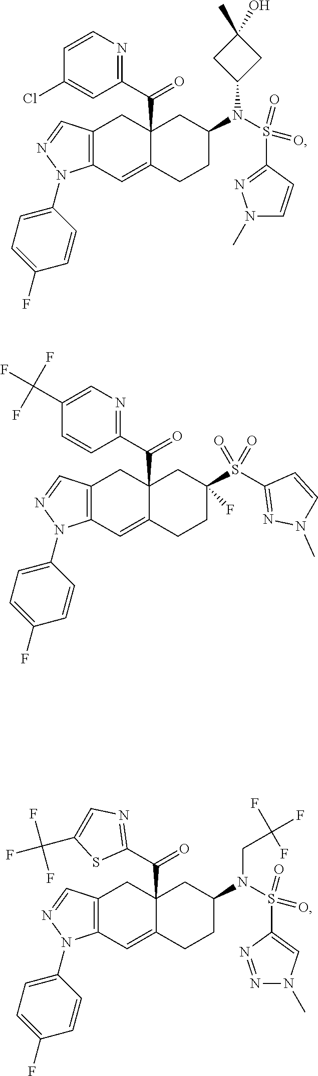 [SMILES: Cn1ccc(S(=O)(=O)N([C@H]2CCC3=Cc4c(cnn4-c4ccc(F)cc4)C[C@]3(C(=O)c3cc(Cl)ccn3)C2)[C@H]2C[C@@](C)(O)C2)n1 ; Cn1ccc(S(=O)(=O)[C@@]2(F)CCC3=Cc4c(cnn4-c4ccc(F)cc4)C[C@]3(C(=O)c3ccc(C(F)(F)F)cn3)C2)n1 ; Cn1cc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)nn1] 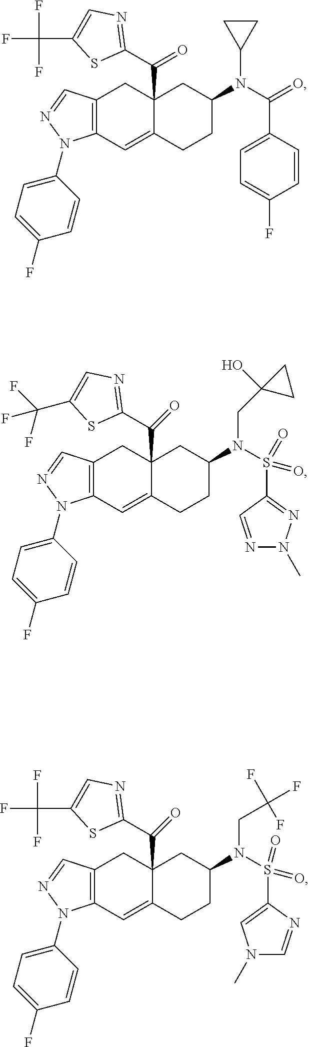 [SMILES: O=C(c1ccc(F)cc1)N(C1CC1)[C@H]1CCC2=Cc3c(cnn3-c3ccc(F)cc3)C[C@]2(C(=O)c2ncc(C(F)(F)F)s2)C1 ; Cn1ncc(S(=O)(=O)N(CC2(O)CC2)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)c1] 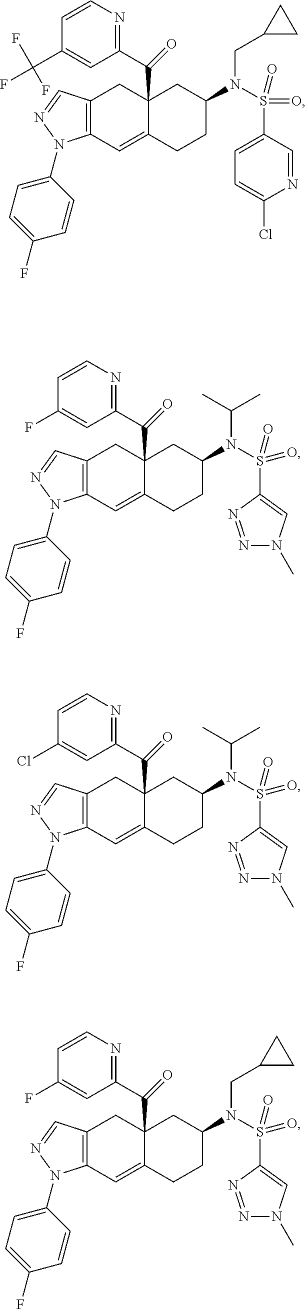 [SMILES: O=C(c1cc(C(F)(F)F)ccn1)[C@]12Cc3cnn(-c4ccc(F)cc4)c3C=C1CC[C@H](N(CC1CC1)S(=O)(=O)c1ccc(Cl)nc1)C2 ; CC(C)N([C@H]1CCC2=Cc3c(cnn3-c3ccc(F)cc3)C[C@]2(C(=O)c2cc(F)ccn2)C1)S(=O)(=O)c1cn(C)nn1 ; CC(C)N([C@H]1CCC2=Cc3c(cnn3-c3ccc(F)cc3)C[C@]2(C(=O)c2cc(Cl)ccn2)C1)S(=O)(=O)c1cn(C)nn1 ; Cn1cc(S(=O)(=O)N(CC2CC2)[C@H]2CCC3=Cc4c(cnn4-c4ccc(F)cc4)C[C@]3(C(=O)c3cc(F)ccn3)C2)nn1] 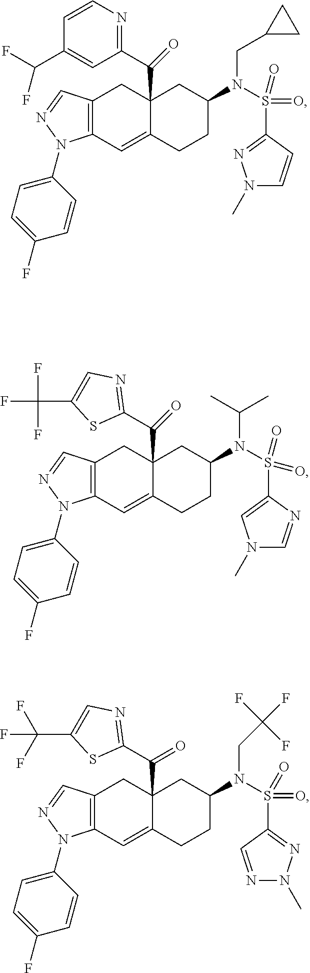 [SMILES: Cn1ccc(S(=O)(=O)N(CC2CC2)[C@H]2CCC3=Cc4c(cnn4-c4ccc(F)cc4)C[C@]3(C(=O)c3cc(C(F)F)ccn3)C2)n1 ; CC(C)N([C@H]1CCC2=Cc3c(cnn3-c3ccc(F)cc3)C[C@]2(C(=O)c2ncc(C(F)(F)F)s2)C1)S(=O)(=O)c1cn(C)cn1 ; Cn1ncc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1] 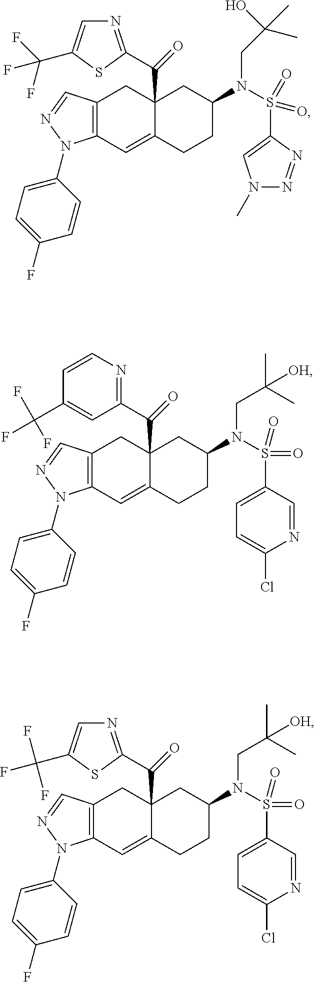 [SMILES: Cn1cc(S(=O)(=O)N(CC(C)(C)O)[C@H]2CCC3=Cc4c(cnn4-c4ccc(F)cc4)C[C@]3(C(=O)c3ncc(C(F)(F)F)s3)C2)nn1 ; CC(C)(O)CN([C@H]1CCC2=Cc3c(cnn3-c3ccc(F)cc3)C[C@]2(C(=O)c2cc(C(F)(F)F)ccn2)C1)S(=O)(=O)c1ccc(Cl)nc1 ; CC(C)(O)CN([C@H]1CCC2=Cc3c(cnn3-c3ccc(F)cc3)C[C@]2(C(=O)c2ncc(C(F)(F)F)s2)C1)S(=O)(=O)c1ccc(Cl)nc1] 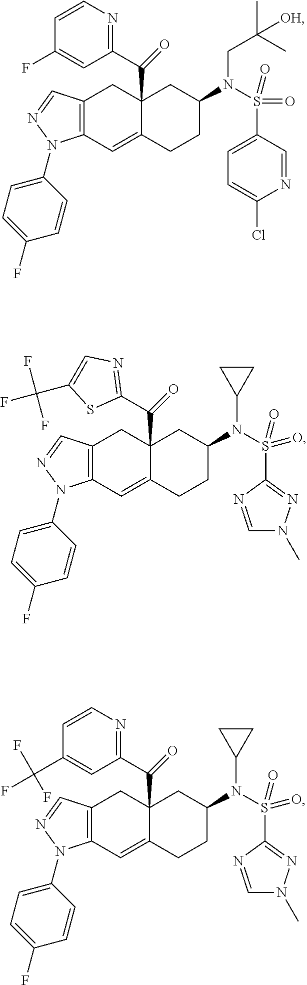 [SMILES: CC(C)(O)CN([C@H]1CCC2=Cc3c(cnn3-c3ccc(F)cc3)C[C@]2(C(=O)c2cc(F)ccn2)C1)S(=O)(=O)c1ccc(Cl)nc1 ; Cn1cnc(S(=O)(=O)N(C2CC2)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(C2CC2)[C@H]2CCC3=Cc4c(cnn4-c4ccc(F)cc4)C[C@]3(C(=O)c3cc(C(F)(F)F)ccn3)C2)n1] 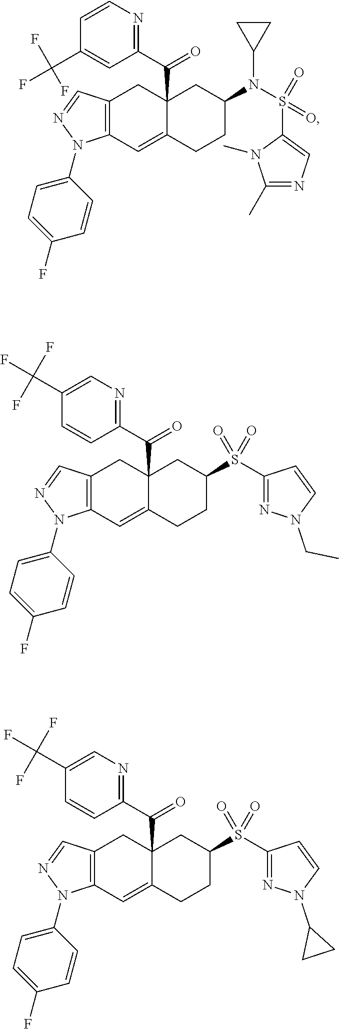 [SMILES: Cc1ncc(S(=O)(=O)N(C2CC2)[C@H]2CCC3=Cc4c(cnn4-c4ccc(F)cc4)C[C@]3(C(=O)c3cc(C(F)(F)F)ccn3)C2)n1C ; CCn1ccc(S(=O)(=O)[C@H]2CCC3=Cc4c(cnn4-c4ccc(F)cc4)C[C@]3(C(=O)c3ccc(C(F)(F)F)cn3)C2)n1 ; O=C(c1ccc(C(F)(F)F)cn1)[C@]12Cc3cnn(-c4ccc(F)cc4)c3C=C1CC[C@H](S(=O)(=O)c1ccn(C3CC3)n1)C2] 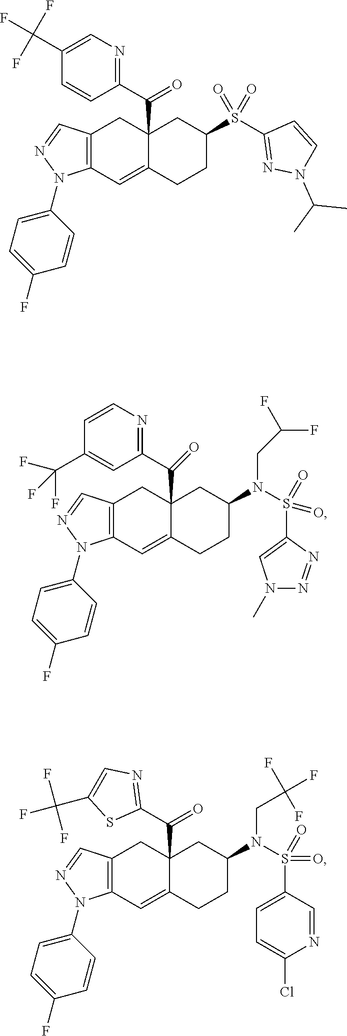 [SMILES: CC(C)n1ccc(S(=O)(=O)[C@H]2CCC3=Cc4c(cnn4-c4ccc(F)cc4)C[C@]3(C(=O)c3ccc(C(F)(F)F)cn3)C2)n1 ; Cn1cc(S(=O)(=O)N(CC(F)F)[C@H]2CCC3=Cc4c(cnn4-c4ccc(F)cc4)C[C@]3(C(=O)c3cc(C(F)(F)F)ccn3)C2)nn1 ; O=C(c1ncc(C(F)(F)F)s1)[C@]12Cc3cnn(-c4ccc(F)cc4)c3C=C1CC[C@H](N(CC(F)(F)F)S(=O)(=O)c1ccc(Cl)nc1)C2] 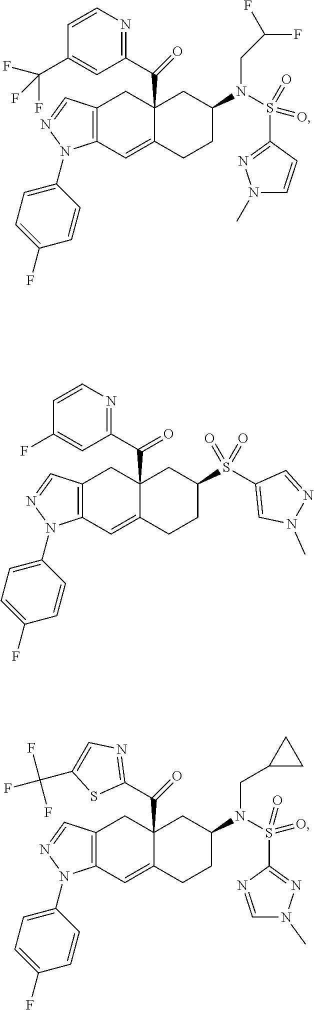 [SMILES: Cn1ccc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1 ; Cn1cc(S(=O)(=O)[C@H]2CCC3=Cc4c(cnn4-c4ccc(F)cc4)C[C@]3(C(=O)c3cc(F)ccn3)C2)cn1 ; Cn1cnc(S(=O)(=O)N(CC2CC2)[C@H]2CCC3=Cc4c(cnn4-c4ccc(F)cc4)C[C@]3(C(=O)c3ncc(C(F)(F)F)s3)C2)n1] 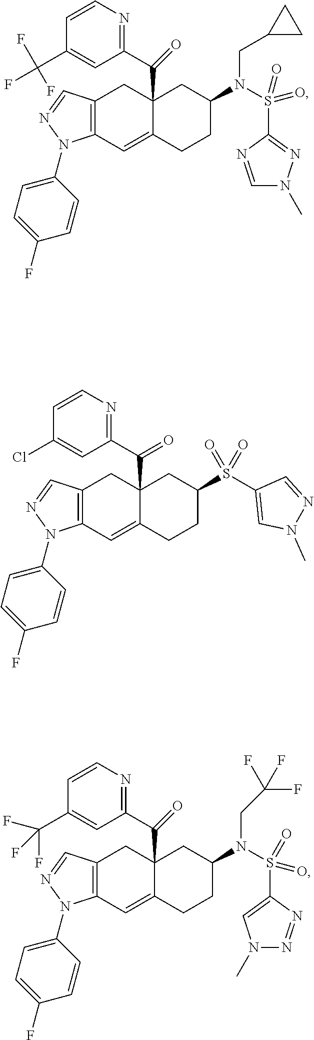 [SMILES: Cn1cnc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1 ; Cn1cc(S(=O)(=O)[C@H]2CCC3=Cc4c(cnn4-c4ccc(F)cc4)C[C@]3(C(=O)c3cc(Cl)ccn3)C2)cn1 ; Cn1cc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)nn1] 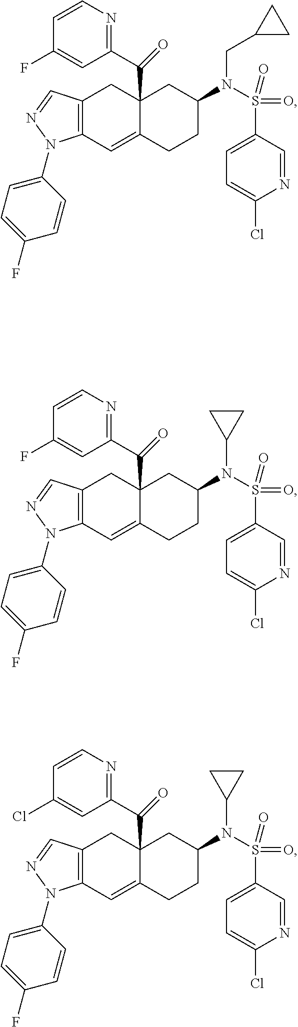 [SMILES: O=C(c1cc(F)ccn1)[C@]12Cc3cnn(-c4ccc(F)cc4)c3C=C1CC[C@H](N(CC1CC1)S(=O)(=O)c1ccc(Cl)nc1)C2 ; O=C(c1cc(F)ccn1)[C@]12Cc3cnn(-c4ccc(F)cc4)c3C=C1CC[C@H](N(C1CC1)S(=O)(=O)c1ccc(Cl)nc1)C2 ; O=C(c1cc(Cl)ccn1)[C@]12Cc3cnn(-c4ccc(F)cc4)c3C=C1CC[C@H](N(C1CC1)S(=O)(=O)c1ccc(Cl)nc1)C2] 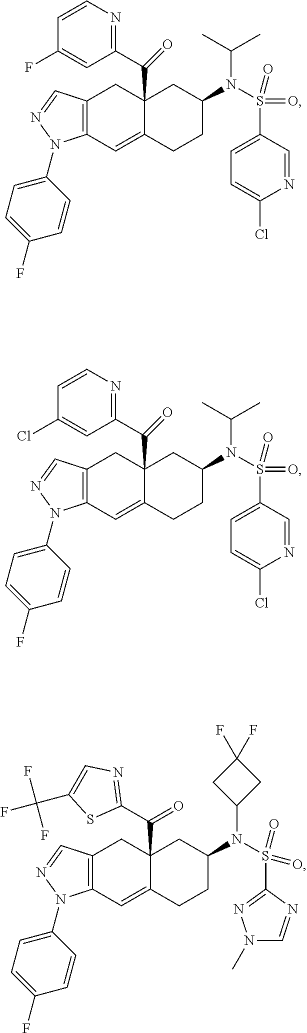 [SMILES: CC(C)N([C@H]1CCC2=Cc3c(cnn3-c3ccc(F)cc3)C[C@]2(C(=O)c2cc(F)ccn2)C1)S(=O)(=O)c1ccc(Cl)nc1 ; CC(C)N([C@H]1CCC2=Cc3c(cnn3-c3ccc(F)cc3)C[C@]2(C(=O)c2cc(Cl)ccn2)C1)S(=O)(=O)c1ccc(Cl)nc1 ; Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)n1] 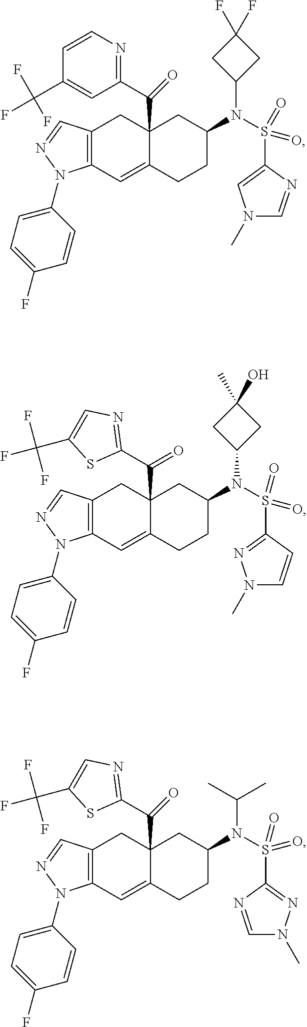 [SMILES: Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)c1 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](C)(O)C2)n1 ; CC(C)N([C@H]1CCC2=Cc3c(cnn3-c3ccc(F)cc3)C[C@]2(C(=O)c2ncc(C(F)(F)F)s2)C1)S(=O)(=O)c1ncn(C)n1] 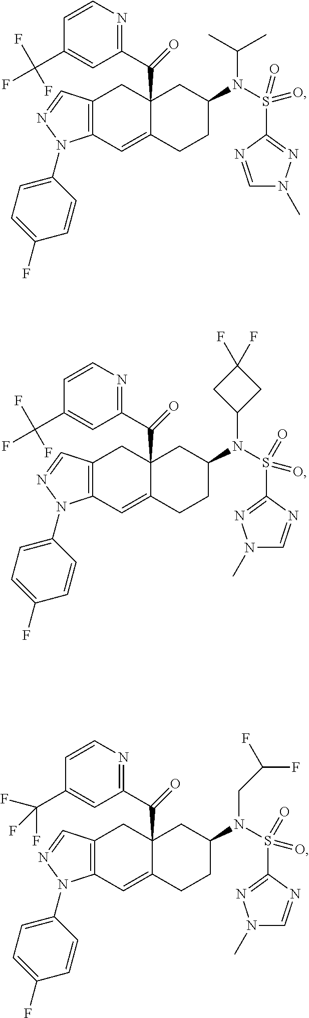 [SMILES: CC(C)N([C@H]1CCC2=Cc3c(cnn3-c3ccc(F)cc3)C[C@]2(C(=O)c2cc(C(F)(F)F)ccn2)C1)S(=O)(=O)c1ncn(C)n1 ; Cn1cnc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)N(CC(F)F)[C@H]2CCC3=Cc4c(cnn4-c4ccc(F)cc4)C[C@]3(C(=O)c3cc(C(F)(F)F)ccn3)C2)n1] 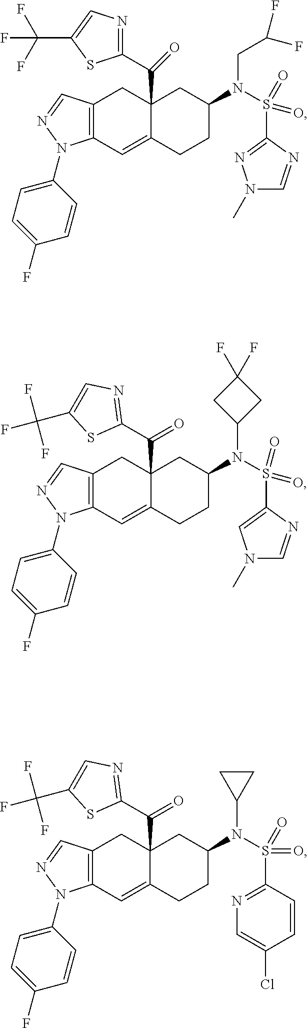 [SMILES: Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)c1 ; O=C(c1ncc(C(F)(F)F)s1)[C@]12Cc3cnn(-c4ccc(F)cc4)c3C=C1CC[C@H](N(C1CC1)S(=O)(=O)c1ccc(Cl)cn1)C2] 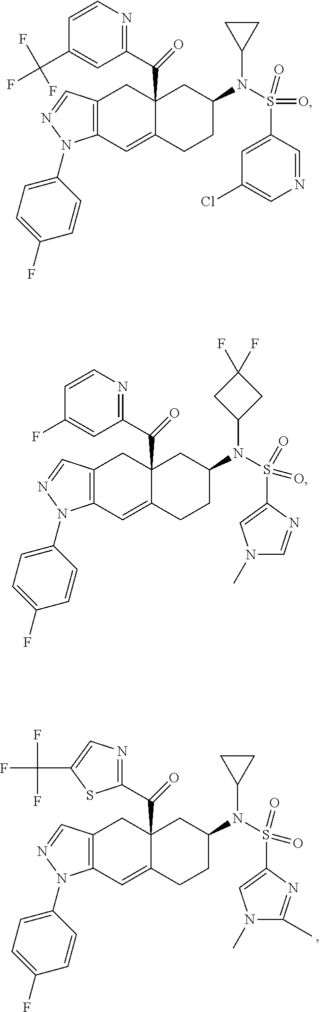 [SMILES: O=C(c1cc(C(F)(F)F)ccn1)[C@]12Cc3cnn(-c4ccc(F)cc4)c3C=C1CC[C@H](N(C1CC1)S(=O)(=O)c1cncc(Cl)c1)C2 ; Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)c1 ; Cc1nc(S(=O)(=O)N(C2CC2)[C@H]2CCC3=Cc4c(cnn4-c4ccc(F)cc4)C[C@]3(C(=O)c3ncc(C(F)(F)F)s3)C2)cn1C] 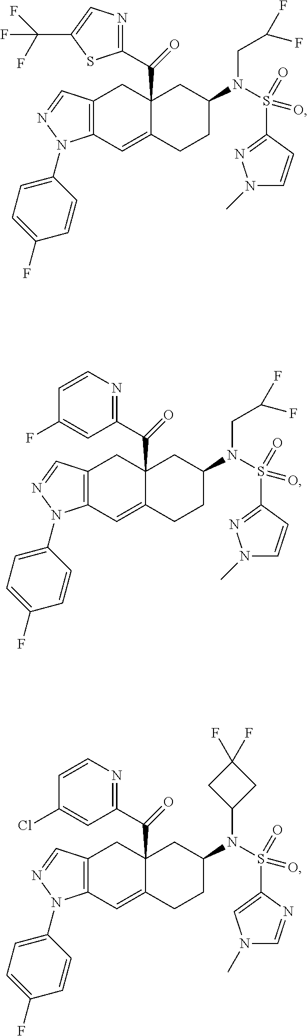 [SMILES: Cn1ccc(S(=O)(=O)N(CC(F)F)[C@H]2CCC3=Cc4c(cnn4-c4ccc(F)cc4)C[C@]3(C(=O)c3ncc(C(F)(F)F)s3)C2)n1 ; Cn1ccc(S(=O)(=O)N(CC(F)F)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)c1] 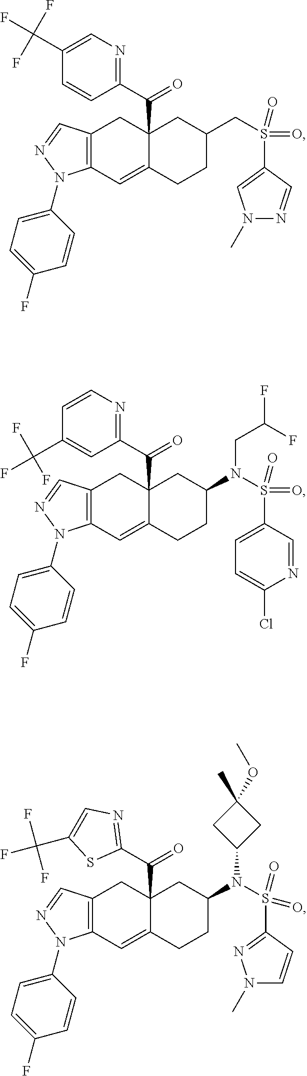 [SMILES: Cn1cc(S(=O)(=O)CC2CCC3=Cc4c(cnn4-c4ccc(F)cc4)C[C@]3(C(=O)c3ccc(C(F)(F)F)cn3)C2)cn1 ; O=C(c1cc(C(F)(F)F)ccn1)[C@]12Cc3cnn(-c4ccc(F)cc4)c3C=C1CC[C@H](N(CC(F)F)S(=O)(=O)c1ccc(Cl)nc1)C2 ; CO[C@]1(C)C[C@H](N([C@H]2CCC3=Cc4c(cnn4-c4ccc(F)cc4)C[C@]3(C(=O)c3ncc(C(F)(F)F)s3)C2)S(=O)(=O)c2ccn(C)n2)C1] 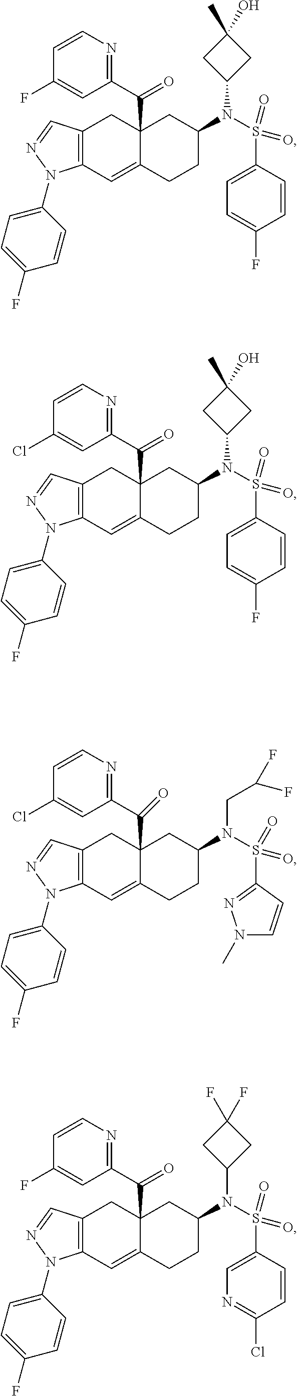 [SMILES: C[C@]1(O)C[C@@H](N([C@H]2CCC3=Cc4c(cnn4-c4ccc(F)cc4)C[C@]3(C(=O)c3cc(F)ccn3)C2)S(=O)(=O)c2ccc(F)cc2)C1 ; C[C@]1(O)C[C@@H](N([C@H]2CCC3=Cc4c(cnn4-c4ccc(F)cc4)C[C@]3(C(=O)c3cc(Cl)ccn3)C2)S(=O)(=O)c2ccc(F)cc2)C1 ; Cn1ccc(S(=O)(=O)N(CC(F)F)[C@H]2CCC3=Cc4c(cnn4-c4ccc(F)cc4)C[C@]3(C(=O)c3cc(Cl)ccn3)C2)n1 ; O=C(c1cc(F)ccn1)[C@]12Cc3cnn(-c4ccc(F)cc4)c3C=C1CC[C@H](N(C1CC(F)(F)C1)S(=O)(=O)c1ccc(Cl)nc1)C2] 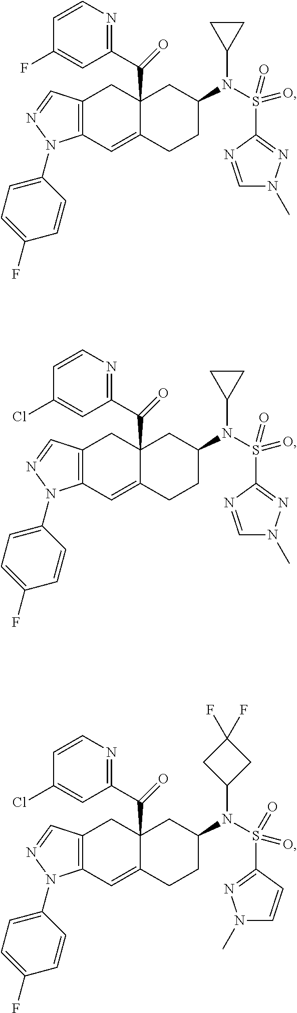 [SMILES: Cn1cnc(S(=O)(=O)N(C2CC2)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC2)[C@H]2CCC3=Cc4c(cnn4-c4ccc(F)cc4)C[C@]3(C(=O)c3cc(Cl)ccn3)C2)n1 ; Cn1ccc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1] 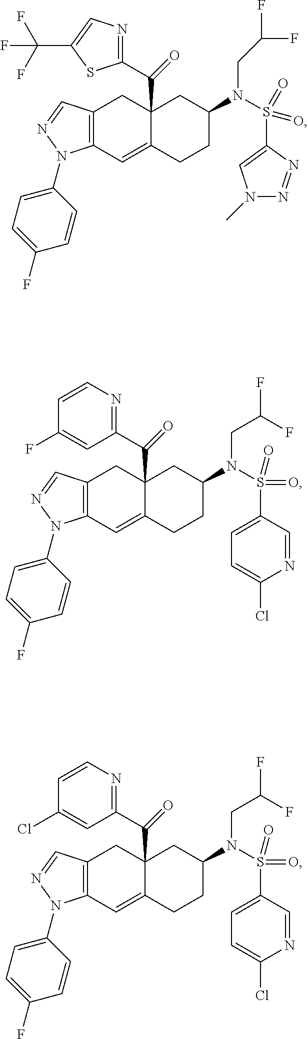 [SMILES: Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)nn1 ; O=C(c1cc(F)ccn1)[C@]12Cc3cnn(-c4ccc(F)cc4)c3C=C1CC[C@H](N(CC(F)F)S(=O)(=O)c1ccc(Cl)nc1)C2 ; O=C(c1cc(Cl)ccn1)[C@]12Cc3cnn(-c4ccc(F)cc4)c3C=C1CC[C@H](N(CC(F)F)S(=O)(=O)c1ccc(Cl)nc1)C2] 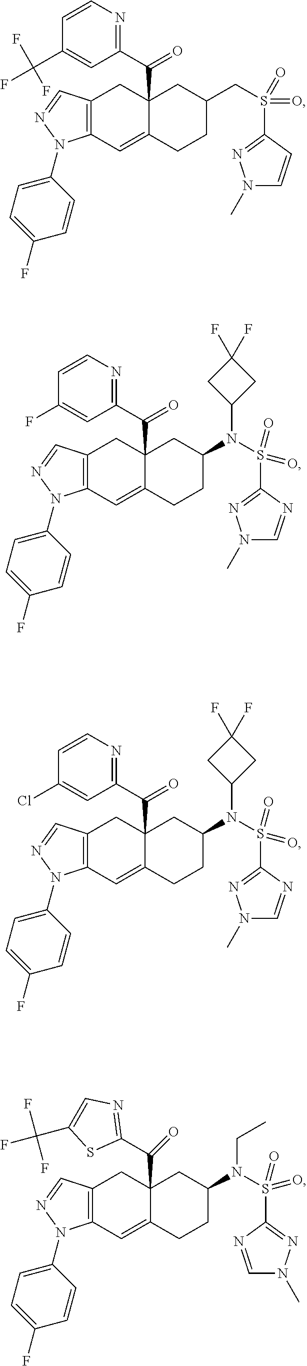 [SMILES: Cn1ccc(S(=O)(=O)CC2CCC3=Cc4c(cnn4-c4ccc(F)cc4)C[C@]3(C(=O)c3cc(C(F)(F)F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(F)ccn3)C2)n1 ; Cn1cnc(S(=O)(=O)N(C2CC(F)(F)C2)[C@H]2CCC3=Cc4c(cnn4-c4ccc(F)cc4)C[C@]3(C(=O)c3cc(Cl)ccn3)C2)n1 ; CCN([C@H]1CCC2=Cc3c(cnn3-c3ccc(F)cc3)C[C@]2(C(=O)c2ncc(C(F)(F)F)s2)C1)S(=O)(=O)c1ncn(C)n1] 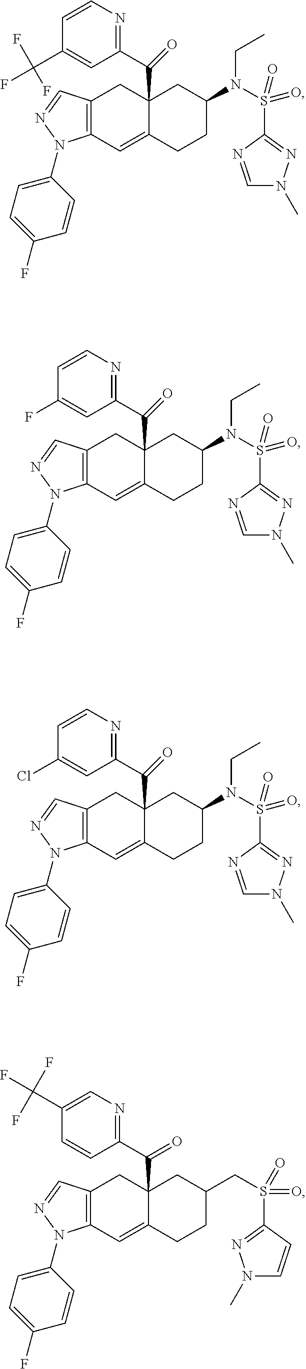 [SMILES: CCN([C@H]1CCC2=Cc3c(cnn3-c3ccc(F)cc3)C[C@]2(C(=O)c2cc(C(F)(F)F)ccn2)C1)S(=O)(=O)c1ncn(C)n1 ; CCN([C@H]1CCC2=Cc3c(cnn3-c3ccc(F)cc3)C[C@]2(C(=O)c2cc(F)ccn2)C1)S(=O)(=O)c1ncn(C)n1 ; CCN([C@H]1CCC2=Cc3c(cnn3-c3ccc(F)cc3)C[C@]2(C(=O)c2cc(Cl)ccn2)C1)S(=O)(=O)c1ncn(C)n1 ; Cn1ccc(S(=O)(=O)CC2CCC3=Cc4c(cnn4-c4ccc(F)cc4)C[C@]3(C(=O)c3ccc(C(F)(F)F)cn3)C2)n1] 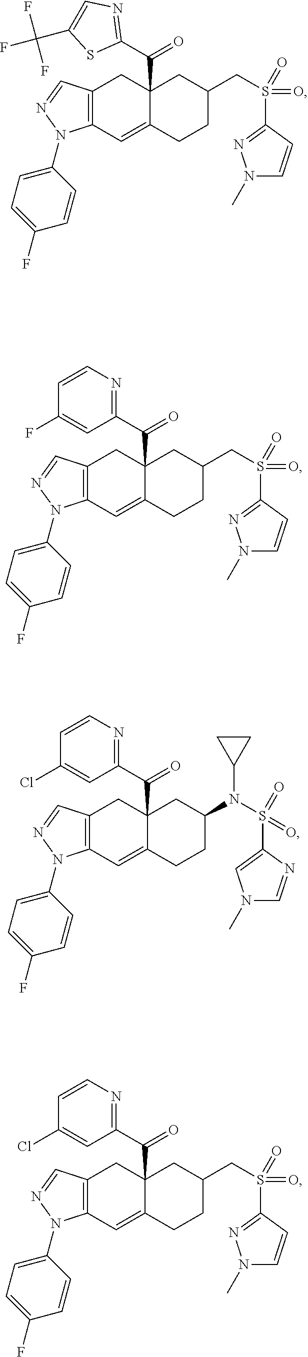 [SMILES: Cn1ccc(S(=O)(=O)CC2CCC3=Cc4c(cnn4-c4ccc(F)cc4)C[C@]3(C(=O)c3ncc(C(F)(F)F)s3)C2)n1 ; Cn1ccc(S(=O)(=O)CC2CCC3=Cc4c(cnn4-c4ccc(F)cc4)C[C@]3(C(=O)c3cc(F)ccn3)C2)n1 ; Cn1cnc(S(=O)(=O)N(C2CC2)[C@H]2CCC3=Cc4c(cnn4-c4ccc(F)cc4)C[C@]3(C(=O)c3cc(Cl)ccn3)C2)c1 ; Cn1ccc(S(=O)(=O)CC2CCC3=Cc4c(cnn4-c4ccc(F)cc4)C[C@]3(C(=O)c3cc(Cl)ccn3)C2)n1] 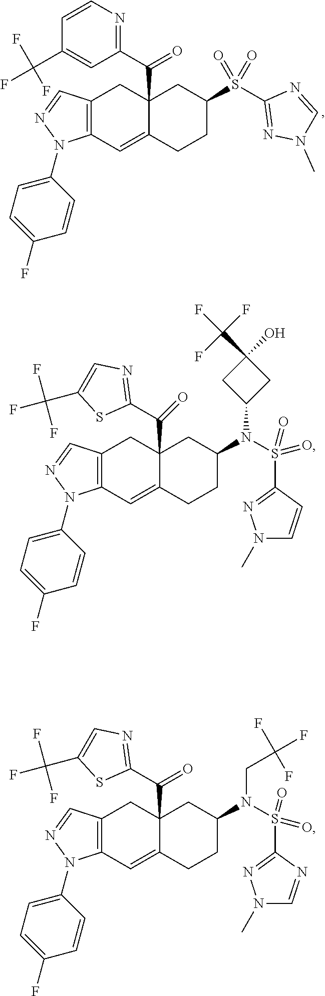 [SMILES: Cn1cnc(S(=O)(=O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@](O)(C(F)(F)F)C2)n1 ; Cn1cnc(S(=O)(=O)N(CC(F)(F)F)[C@H]2CCC3=Cc4c(cnn4-c4ccc(F)cc4)C[C@]3(C(=O)c3ncc(C(F)(F)F)s3)C2)n1] 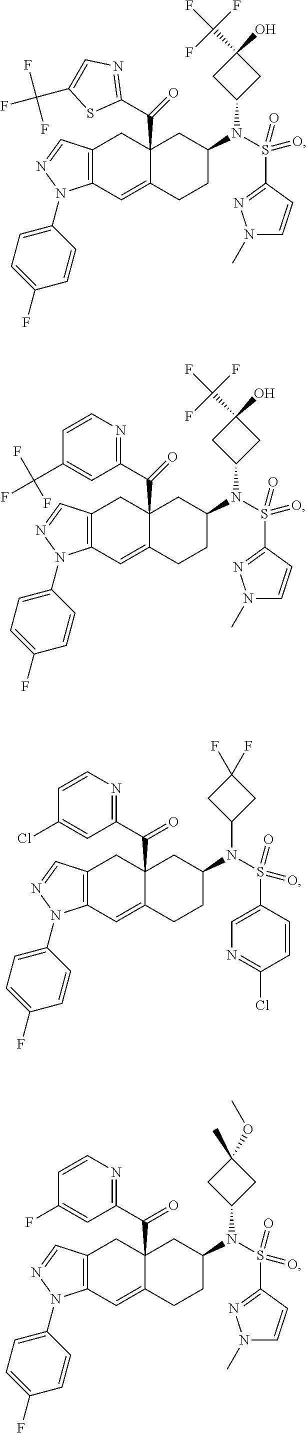 [SMILES: Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@](O)(C(F)(F)F)C2)n1 ; Cn1ccc(S(=O)(=O)N([C@H]2CCC3=Cc4c(cnn4-c4ccc(F)cc4)C[C@]3(C(=O)c3cc(C(F)(F)F)ccn3)C2)[C@H]2C[C@@](O)(C(F)(F)F)C2)n1 ; O=C(c1cc(Cl)ccn1)[C@]12Cc3cnn(-c4ccc(F)cc4)c3C=C1CC[C@H](N(C1CC(F)(F)C1)S(=O)(=O)c1ccc(Cl)nc1)C2 ; CO[C@]1(C)C[C@H](N([C@H]2CCC3=Cc4c(cnn4-c4ccc(F)cc4)C[C@]3(C(=O)c3cc(F)ccn3)C2)S(=O)(=O)c2ccn(C)n2)C1] 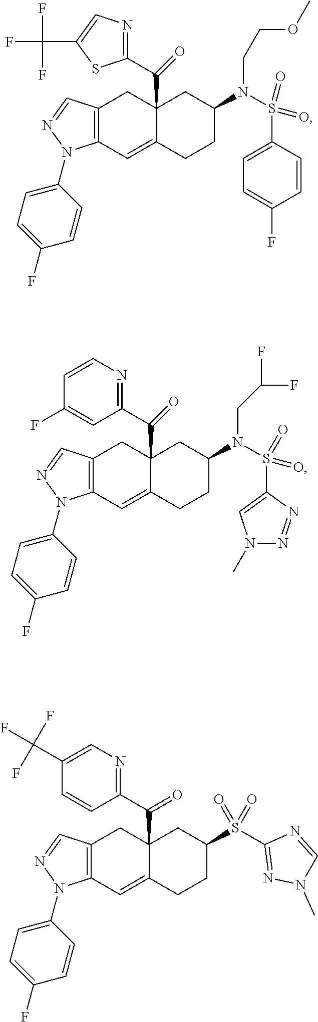 [SMILES: COCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1 ; Cn1cc(S(=O)(=O)N(CC(F)F)[C@H]2CCC3=Cc4c(cnn4-c4ccc(F)cc4)C[C@]3(C(=O)c3cc(F)ccn3)C2)nn1 ; Cn1cnc(S(=O)(=O)[C@H]2CCC3=Cc4c(cnn4-c4ccc(F)cc4)C[C@]3(C(=O)c3ccc(C(F)(F)F)cn3)C2)n1] 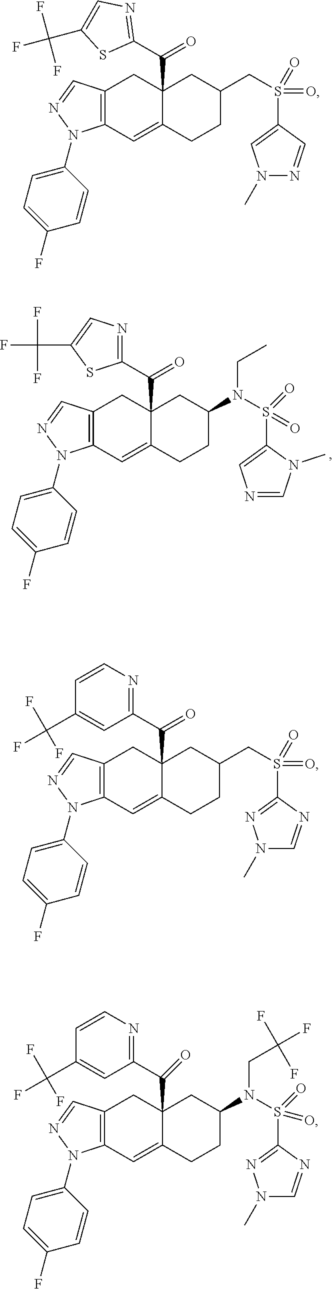 [SMILES: Cn1cc(S(=O)(=O)CC2CCC3=Cc4c(cnn4-c4ccc(F)cc4)C[C@]3(C(=O)c3ncc(C(F)(F)F)s3)C2)cn1 ; CCN([C@H]1CCC2=Cc3c(cnn3-c3ccc(F)cc3)C[C@]2(C(=O)c2ncc(C(F)(F)F)s2)C1)S(=O)(=O)c1cncn1C ; Cn1cnc(S(=O)(=O)CC2CCC3=Cc4c(cnn4-c4ccc(F)cc4)C[C@]3(C(=O)c3cc(C(F)(F)F)ccn3)C2)n1 ; Cn1cnc(S(=O)(=O)N(CC(F)(F)F)[C@H]2CCC3=Cc4c(cnn4-c4ccc(F)cc4)C[C@]3(C(=O)c3cc(C(F)(F)F)ccn3)C2)n1] 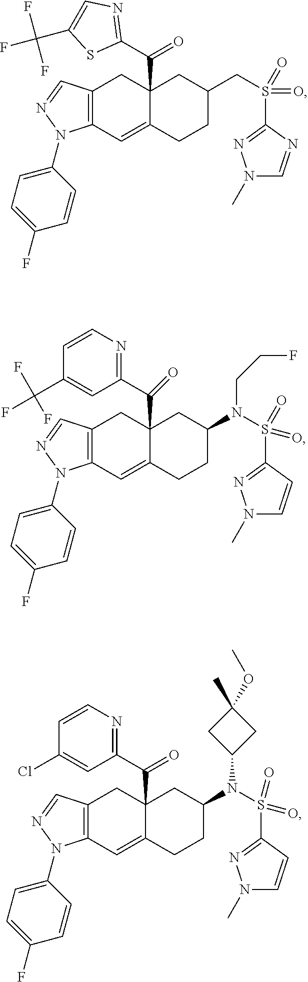 [SMILES: Cn1cnc(S(=O)(=O)CC2CCC3=Cc4c(cnn4-c4ccc(F)cc4)C[C@]3(C(=O)c3ncc(C(F)(F)F)s3)C2)n1 ; Cn1ccc(S(=O)(=O)N(CCF)[C@H]2CCC3=Cc4c(cnn4-c4ccc(F)cc4)C[C@]3(C(=O)c3cc(C(F)(F)F)ccn3)C2)n1 ; CO[C@]1(C)C[C@H](N([C@H]2CCC3=Cc4c(cnn4-c4ccc(F)cc4)C[C@]3(C(=O)c3cc(Cl)ccn3)C2)S(=O)(=O)c2ccn(C)n2)C1] 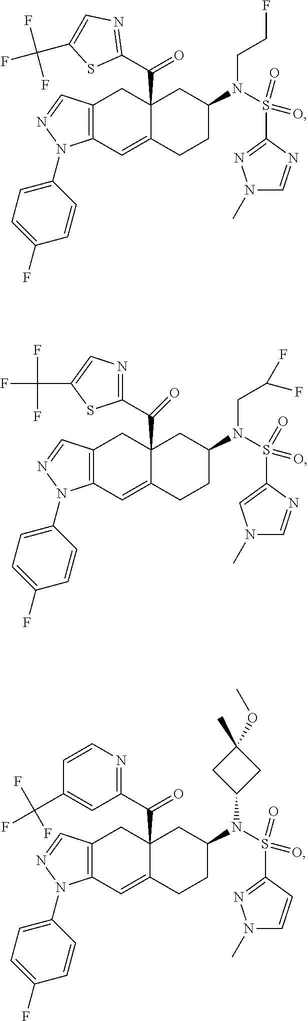 [SMILES: Cn1cnc(S(=O)(=O)N(CCF)[C@H]2CCC3=Cc4c(cnn4-c4ccc(F)cc4)C[C@]3(C(=O)c3ncc(C(F)(F)F)s3)C2)n1 ; Cn1cnc(S(=O)(=O)N(CC(F)F)[C@H]2CCC3=Cc4c(cnn4-c4ccc(F)cc4)C[C@]3(C(=O)c3ncc(C(F)(F)F)s3)C2)c1 ; CO[C@]1(C)C[C@H](N([C@H]2CCC3=Cc4c(cnn4-c4ccc(F)cc4)C[C@]3(C(=O)c3cc(C(F)(F)F)ccn3)C2)S(=O)(=O)c2ccn(C)n2)C1] 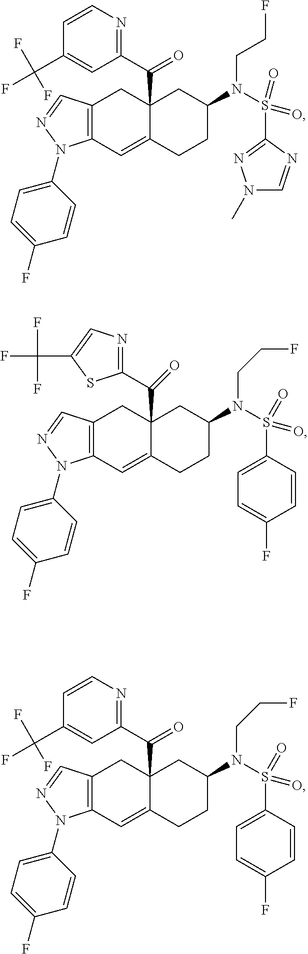 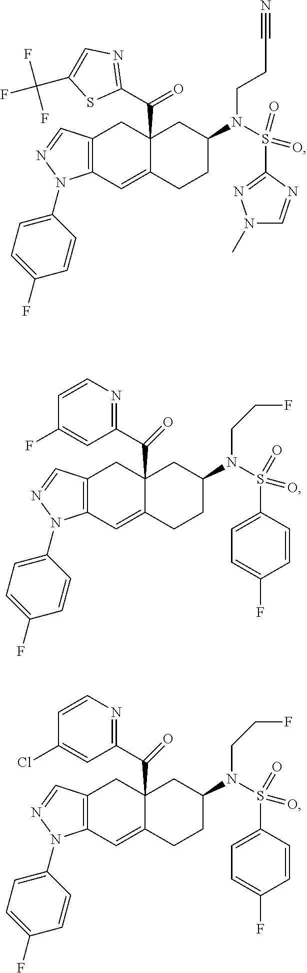 [SMILES: Cn1cnc(S(=O)(=O)N(CCC#N)[C@H]2CCC3=Cc4c(cnn4-c4ccc(F)cc4)C[C@]3(C(=O)c3ncc(C(F)(F)F)s3)C2)n1 ; O=C(c1cc(F)ccn1)[C@]12Cc3cnn(-c4ccc(F)cc4)c3C=C1CC[C@H](N(CCF)S(=O)(=O)c1ccc(F)cc1)C2 ; O=C(c1cc(Cl)ccn1)[C@]12Cc3cnn(-c4ccc(F)cc4)c3C=C1CC[C@H](N(CCF)S(=O)(=O)c1ccc(F)cc1)C2] 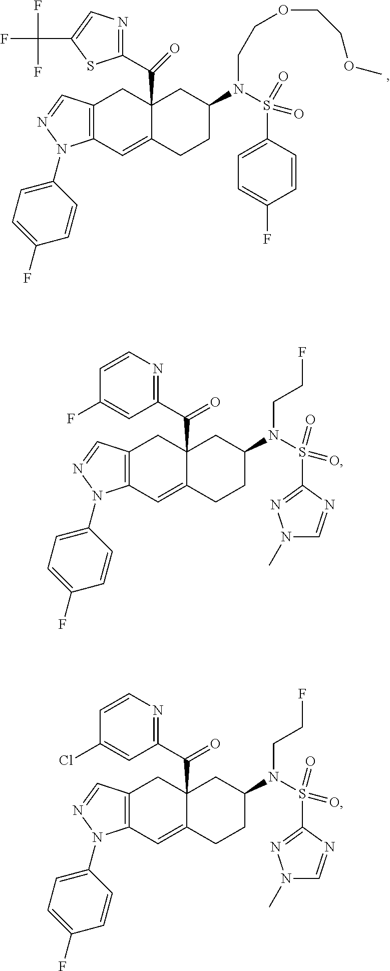 [SMILES: COCCOCCN([C@H]1CCC2=Cc3c(cnn3-c3ccc(F)cc3)C[C@]2(C(=O)c2ncc(C(F)(F)F)s2)C1)S(=O)(=O)c1ccc(F)cc1 ; Cn1cnc(S(=O)(=O)N(CCF)[C@H]2CCC3=Cc4c(cnn4-c4ccc(F)cc4)C[C@]3(C(=O)c3cc(F)ccn3)C2)n1 ; Cn1cnc(S(=O)(=O)N(CCF)[C@H]2CCC3=Cc4c(cnn4-c4ccc(F)cc4)C[C@]3(C(=O)c3cc(Cl)ccn3)C2)n1] 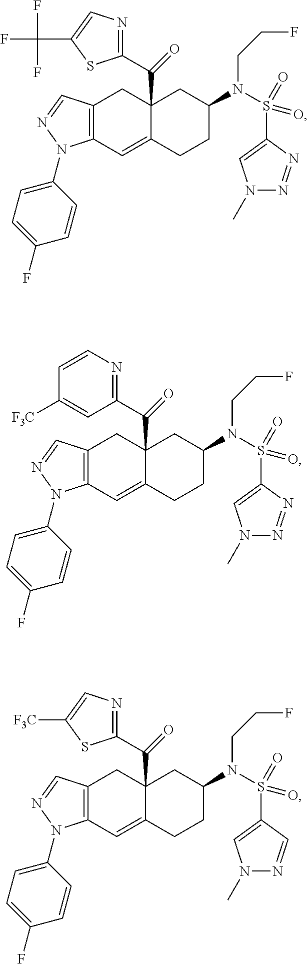 [SMILES: Cn1cc(S(=O)(=O)N(CCF)[C@H]2CCC3=Cc4c(cnn4-c4ccc(F)cc4)C[C@]3(C(=O)c3ncc(C(F)(F)F)s3)C2)nn1 ; Cn1cc(S(=O)(=O)N(CCF)[C@H]2CCC3=Cc4c(cnn4-c4ccc(F)cc4)C[C@]3(C(=O)c3cc(C(F)(F)F)ccn3)C2)nn1 ; Cn1cc(S(=O)(=O)N(CCF)[C@H]2CCC3=Cc4c(cnn4-c4ccc(F)cc4)C[C@]3(C(=O)c3ncc(C(F)(F)F)s3)C2)cn1] 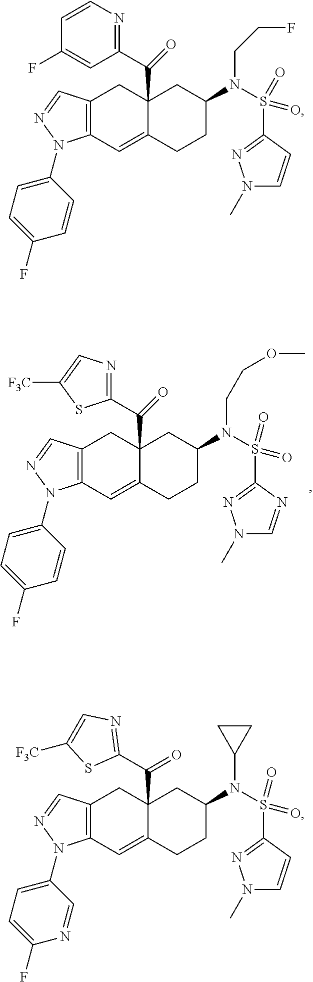 [SMILES: Cn1ccc(S(=O)(=O)N(CCF)[C@H]2CCC3=Cc4c(cnn4-c4ccc(F)cc4)C[C@]3(C(=O)c3cc(F)ccn3)C2)n1 ; COCCN([C@H]1CCC2=Cc3c(cnn3-c3ccc(F)cc3)C[C@]2(C(=O)c2ncc(C(F)(F)F)s2)C1)S(=O)(=O)c1ncn(C)n1 ; Cn1ccc(S(=O)(=O)N(C2CC2)[C@H]2CCC3=Cc4c(cnn4-c4ccc(F)nc4)C[C@]3(C(=O)c3ncc(C(F)(F)F)s3)C2)n1] 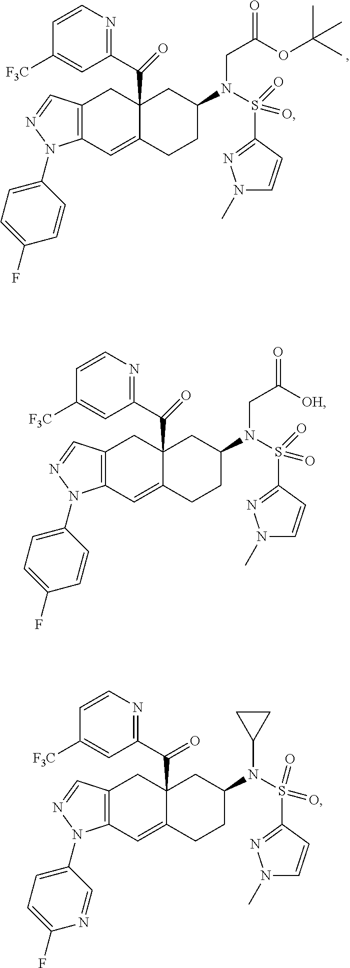 [SMILES: Cn1ccc(S(=O)(=O)N(CC(=O)OC(C)(C)C)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(CC(=O)O)[C@H]2CCC3=Cc4c(cnn4-c4ccc(F)cc4)C[C@]3(C(=O)c3cc(C(F)(F)F)ccn3)C2)n1 ; Cn1ccc(S(=O)(=O)N(C2CC2)[C@H]2CCC3=Cc4c(cnn4-c4ccc(F)nc4)C[C@]3(C(=O)c3cc(C(F)(F)F)ccn3)C2)n1] 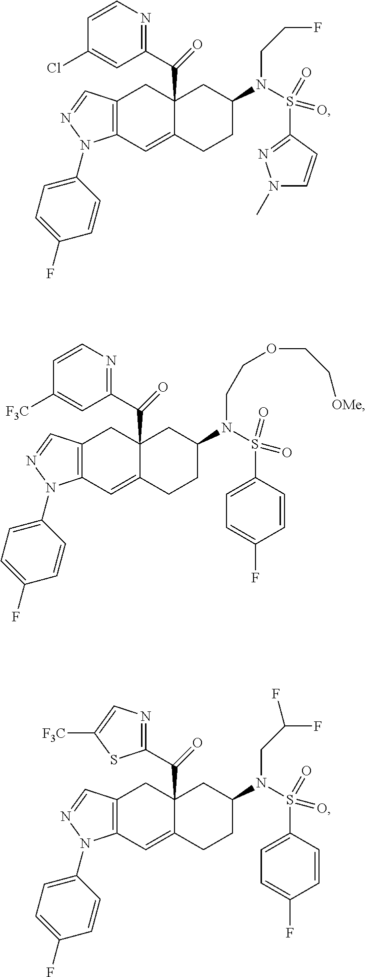 [SMILES: Cn1ccc(S(=O)(=O)N(CCF)[C@H]2CCC3=Cc4c(cnn4-c4ccc(F)cc4)C[C@]3(C(=O)c3cc(Cl)ccn3)C2)n1 ; COCCOCCN([C@H]1CCC2=Cc3c(cnn3-c3ccc(F)cc3)C[C@]2(C(=O)c2cc(C(F)(F)F)ccn2)C1)S(=O)(=O)c1ccc(F)cc1 ; O=C(c1ncc(C(F)(F)F)s1)[C@]12Cc3cnn(-c4ccc(F)cc4)c3C=C1CC[C@H](N(CC(F)F)S(=O)(=O)c1ccc(F)cc1)C2] 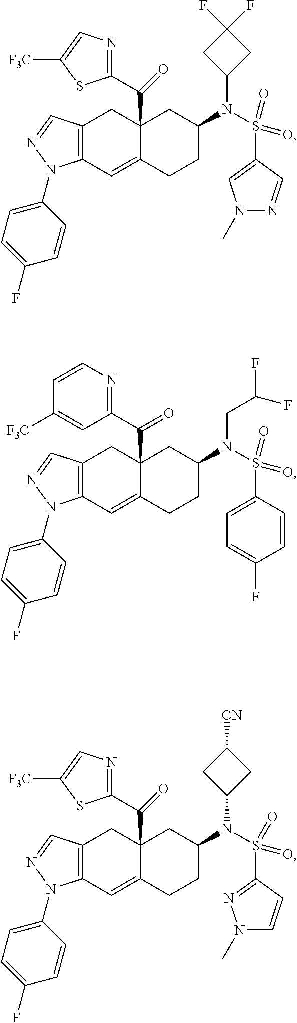 [SMILES: Cn1cc(S(=O)(=O)N(C2CC(F)(F)C2)[C@H]2CCC3=Cc4c(cnn4-c4ccc(F)cc4)C[C@]3(C(=O)c3ncc(C(F)(F)F)s3)C2)cn1 ; O=C(c1cc(C(F)(F)F)ccn1)[C@]12Cc3cnn(-c4ccc(F)cc4)c3C=C1CC[C@H](N(CC(F)F)S(=O)(=O)c1ccc(F)cc1)C2 ; Cn1ccc(S(=O)(=O)N([C@H]2CCC3=Cc4c(cnn4-c4ccc(F)cc4)C[C@]3(C(=O)c3ncc(C(F)(F)F)s3)C2)[C@H]2C[C@@H](C#N)C2)n1] 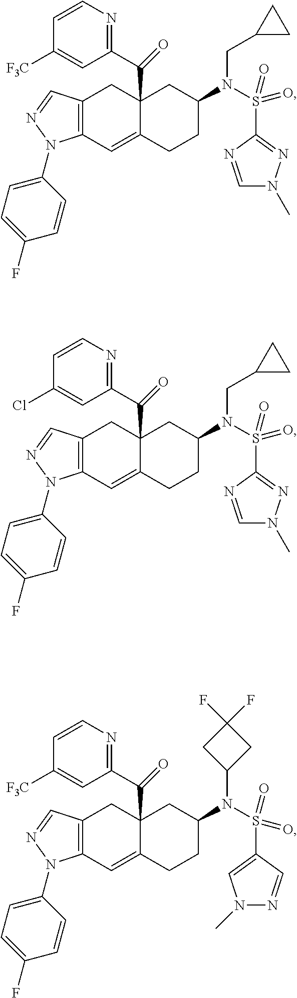 [SMILES: Cn1cnc(S(=O)(=O)N(CC2CC2)[C@H]2CCC3=Cc4c(cnn4-c4ccc(F)cc4)C[C@]3(C(=O)c3cc(C(F)(F)F)ccn3)C2)n1 ; Cn1cnc(S(=O)(=O)N(CC2CC2)[C@H]2CCC3=Cc4c(cnn4-c4ccc(F)cc4)C[C@]3(C(=O)c3cc(Cl)ccn3)C2)n1 ; Cn1cc(S(=O)(=O)N(C2CC(F)(F)C2)[C@H]2CCC3=Cc4c(cnn4-c4ccc(F)cc4)C[C@]3(C(=O)c3cc(C(F)(F)F)ccn3)C2)cn1] 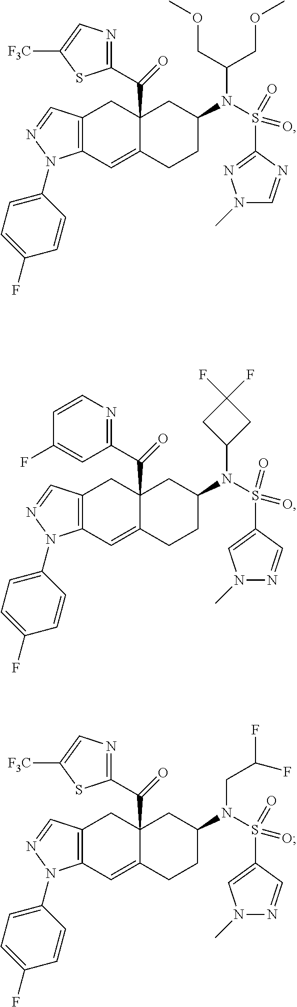 [SMILES: COCC(COC)N([C@H]1CCC2=Cc3c(cnn3-c3ccc(F)cc3)C[C@]2(C(=O)c2ncc(C(F)(F)F)s2)C1)S(=O)(=O)c1ncn(C)n1 ; Cn1cc(S(=O)(=O)N(C2CC(F)(F)C2)[C@H]2CCC3=Cc4c(cnn4-c4ccc(F)cc4)C[C@]3(C(=O)c3cc(F)ccn3)C2)cn1 ; Cn1cc(S(=O)(=O)N(CC(F)F)[C@H]2CCC3=Cc4c(cnn4-c4ccc(F)cc4)C[C@]3(C(=O)c3ncc(C(F)(F)F)s3)C2)cn1]
or a pharmaceutically acceptable salt, solvate, or stereoisomer thereof.

59. (canceled)

60. (canceled)

61. (canceled)

62. A pharmaceutical composition comprising a compound of claim 1, or a pharmaceutically acceptable salt, solvate, or stereoisomer thereof, and at least one pharmaceutically acceptable excipient.

63. A method for treating or preventing cancer in a subject, the method comprising administering a therapeutically effective amount of a compound of claim 1, or a pharmaceutically acceptable salt, solvate, or stereoisomer thereof, to the subject in need thereof.

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)
